(19) United States
(12) Patent Application Publication
(10) Pub. No.: US 2024/0349599 A1
(43) Pub. Date: Oct. 17, 2024

(54) NOVEL COMPOUND AND ORGANIC LIGHT EMITTING DEVICE COMPRISING THE SAME

(71) Applicant: LG CHEM, LTD., Seoul (KR)

(72) Inventors: Jung Min Yoon, Daejeon (KR); Sung Kil Hong, Daejeon (KR); Dong Uk Heo, Daejeon (KR); Miyeon Han, Daejeon (KR); Jae Tak Lee, Daejeon (KR); Heekyung Yun, Daejeon (KR)

(21) Appl. No.: 18/294,486

(22) PCT Filed: Aug. 11, 2022
(86) PCT No.: PCT/KR2022/012058

§ 371 Date: Feb. 1, 2024

(30) Foreign Application Priority Data
Aug. 12, 2021 (KR) 10-2021-0106886

(51) Int. Cl. H10K 85/60; C07D 251/24; C09K 11/06; H10K 50/16; H10K 50/17

(52) U.S. Cl. CPC H10K 85/654; C07D 251/24; C09K 11/06; H10K 85/622; H10K 85/626; C09K 2211/1018; H10K 50/16; H10K 50/171

(57) ABSTRACT

A compound of Chemical Formula 1:

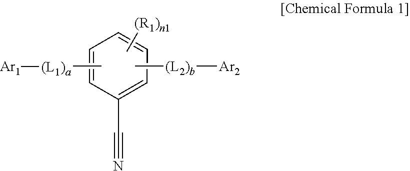

wherein: L₁ and L₂ are each independently a substituted or unsubstituted C₆₋₆₀ arylene, each R₁ is independently hydrogen, deuterium, halogen, cyano, or a substituted or unsubstituted: C₁₋₆₀ alkyl, C₂₋₆₀ alkenyl, C₂₋₆₀ alkynyl, C₃₋₆₀ cycloalkyl, C₆₋₆₀ aryl or C₂₋₆₀ heteroaryl containing one or more of N, O and S; one of Ar₁ and Ar₂ is a substituent of Chemical Formula 2, and the other is a substituted or unsubstituted C₁₄₋₆₀ aromatic fused polycyclic ring:

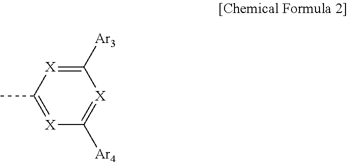

wherein: each X is independently N or CH, provided that at least one X is N; and Ar₃ and Ar₄ are each independently a substituted or unsubstituted C₆₋₆₀ aryl or C₂₋₆₀ heteroaryl containing one or more of N, O and S, and the other substituents are as defined in the specification; and an organic light emitting device comprising the same.

### CROSS-REFERENCE TO RELATED APPLICATION(S)

This application is a National Stage Application of International Application No. PCT/KR2022/012058 filed on Aug. 11, 2022, which claims the benefit of and priority to Korean Patent Application No. 10⁻²⁰²¹-0106886, filed on Aug. 12, 2021 in the Korean Intellectual Property Office, respectively, the disclosures of which are incorporated herein by reference in their entirety.

### TECHNICAL FIELD

The present disclosure relates to a novel compound and an organic light emitting device including the same.

### BACKGROUND

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer can be formed of a hole injection layer, a hole transport layer, a light emitting layer, an electron transport layer, an electron injection layer, and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continuing need for the development of new materials for the organic materials used in the organic light emitting devices as described above.

### BACKGROUND ART LITERATURE

### Patent Literature

(Patent Literature 0001) Korean Unexamined Patent Publication No. 10⁻²⁰⁰⁰-0051826

### BRIEF DESCRIPTION

### Technical Problem

The present disclosure relates to a novel compound and an organic light emitting device including the same.

### Technical Solution

In the present disclosure, provided is a compound of the following Chemical Formula 1:

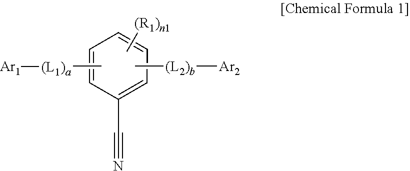

wherein in Chemical Formula 1:
L₁ and L₂ are each independently a substituted or unsubstituted C₆₋₆₀ arylene;
each R₁ is independently a hydrogen, deuterium, halogen, cyano, substituted or unsubstituted C₁₋₆₀ alkyl, substituted or unsubstituted C₁₋₆₀ alkoxy, substituted or unsubstituted C₂₋₆₀ alkenyl, substituted or unsubstituted C₂₋₆₀ alkynyl, substituted or unsubstituted C₃₋₆₀ cycloalkyl, substituted or unsubstituted C₆₋₆₀ aryl, or substituted or unsubstituted C₂₋₆₀ heteroaryl containing any one or more heteroatoms selected from the group consisting of N, O and S;

a and b are each independently 1 or 2;

n1 is an integer from 0 to 3;

one of Ar₁ and Ar₂ is a substituent represented by the following Chemical Formula 2, and the other of Ar₁ and Ar₂ is a substituted or unsubstituted C₁₄₋₆₀ aromatic fused polycyclic ring:

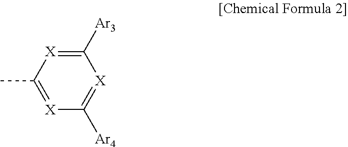

wherein in Chemical Formula 2:
each X is independently N or CH, provided that at least one X is N; and
Ar₃ and Ar₄ are each independently a substituted or unsubstituted C₆₋₆₀ aryl; or a substituted or unsubstituted C₂₋₆₀ heteroaryl containing any one or more heteroatoms selected from the group consisting of N, O and S.

In addition, also provided is an organic light emitting device including: a first electrode; a second electrode that is opposite to the first electrode; and one or more organic material layers that are between the first electrode and the second electrode, wherein at least one organic material layer of the one or more organic material layers includes the compound of Chemical Formula 1.

### Advantageous Effects

The compound of Chemical Formula 1 can be used as a material for an organic material layer of an organic light emitting device, and can improve efficiency, low driving voltage, and/or lifespan of the organic light emitting device. In particular, the compound of Chemical Formula 1 can be used as a material for hole injection, hole transport, hole injection and transport, electron blocking, light emission, hole blocking, electron transport, electron injection, or electron injection and transport.

### BRIEF DESCRIPTION OF DRAWINGS

FIG. 1 shows an example of an organic light emitting device including a substrate 1, an anode 2, a light emitting layer 3, and a cathode 4.

FIG. 2 shows an example of an organic light emitting device including a substrate 1, an anode 2, a hole injection layer 5, a first hole transport layer 6, a second hole transport layer 7, a light emitting layer 3, an electron injection and transport layer 8, and a cathode 4.

### DETAILED DESCRIPTION

Hereinafter, embodiments of the present disclosure will be described in more detail to facilitate understanding of the invention.

As used herein, the notation

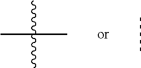

or means a bond linked to another substituent group.

As used herein, the term “substituted or unsubstituted” means being unsubstituted or substituted with one or more substituents selected from the group consisting of deuterium, a halogen group, a nitrile group, a nitro group, a hydroxyl group, a carbonyl group, an ester group, an imide group, an amino group, a phosphine oxide group, an alkoxy group, an aryloxy group, an alkylthioxy group, an arylthioxy group, an alkylsulfoxy group, an arylsulfoxy group, a silyl group, a boron group, an alkyl group, a cycloalkyl group, an alkenyl group, an aryl group, an aralkyl group, an aralkenyl group, an alkylaryl group, an alkylamine group, an aralkylamine group, a heteroarylamine group, an arylamine group, an arylphosphine group; and a heterocyclic group containing at least one of N, O and S as heteroatoms, or being unsubstituted or substituted with a substituent in which two or more substituents of the above-exemplified substituents are connected. For example, “a substituent in which two or more substituents are connected” can be a biphenyl group. That is, a biphenyl group can be an aryl group, or it can also be interpreted as a substituent in which two phenyl groups are connected.

In the present disclosure, the carbon number of a carbonyl group is not particularly limited, but is preferably 1 to 40. Specifically, the carbonyl group can be a group having the following structural formulae, but is not limited thereto:

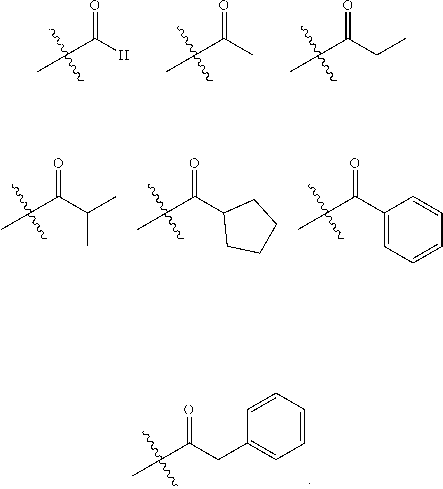

In the present disclosure, for an ester group, the oxygen of the ester group can be substituted with a straight-chain, branched-chain, or cyclic alkyl group having 1 to 25 carbon atoms, or an aryl group having 6 to 25 carbon atoms. Specifically, the ester group can be a group having the following structural formulae, but is not limited thereto:

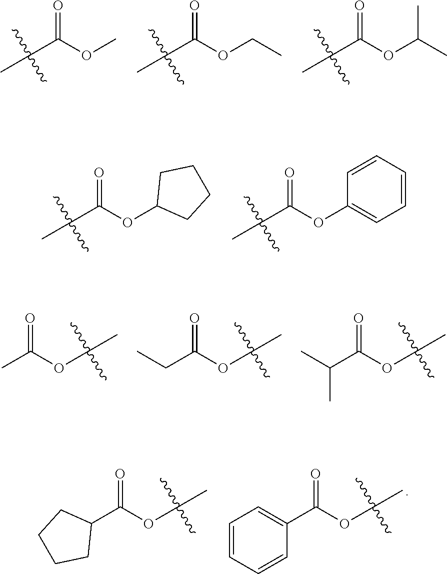

In the present disclosure, the carbon number of an imide group is not particularly limited, but is preferably 1 to 25. Specifically, the imide group can be a group having the following structural formulae, but is not limited thereto:

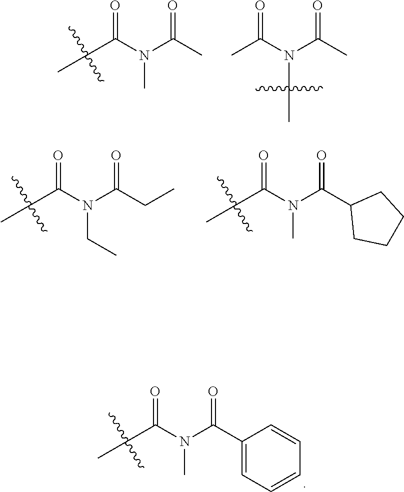

In the present disclosure, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but is not limited thereto.

In the present disclosure, a boron group specifically includes a trimethylboron group, a triethylboron group, a t-butyldimethylboron group, a triphenylboron group, a phenylboron group and the like, but is not limited thereto.

In the present disclosure, examples of a halogen group include fluorine, chlorine, bromine, or iodine.

In the present disclosure, the alkyl group can be straight-chain, or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, the carbon number of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 4-methylhexyl, 5-methylhexyl, and the like, but are not limited thereto.

In the present disclosure, the alkenyl group can be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, a cycloalkyl group is not particularly limited, but the carbon number thereof is preferably 3 to 60. According to one embodiment, the carbon number of the cycloalkyl group is 3 to 30. According to another embodiment, the carbon number of the cycloalkyl group is 3 to 20. According to another embodiment, the carbon number of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it can be a monocyclic aryl group or a polycyclic aryl group. According to one embodiment, the carbon number of the aryl group is 6 to 30. According to one embodiment, the carbon number of the aryl group is 6 to 20. The monocyclic aryl group includes a phenyl group, a biphenyl group, a terphenyl group and the like, but is not limited thereto. The polycyclic aryl group includes a naphthyl group, an anthracenyl group, a phenanthryl group, a pyrenyl group, a perylenyl group, a chrysenyl group, a fluorenyl group, and the like, but is not limited thereto.

In the present disclosure, a fluorenyl group can be substituted, and two substituents can be bonded to each other to form a spiro structure. In the case where the fluorenyl group is substituted,

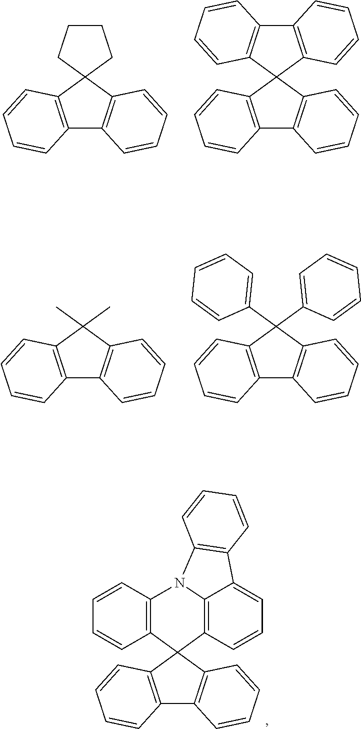

and the like can be formed. However, the structure is not limited thereto.

In the present disclosure, a heterocyclic group is a heterocyclic group containing at least one of N, O, Si and S as a heterogeneous element, and the carbon number thereof is not particularly limited, but is preferably 2 to 60. Examples of the heterocyclic group include a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group, and the arylamine group is the same as the aforementioned examples of the aryl group. In the present disclosure, the alkyl group in the aralkyl group, the alkylaryl group and the alkylamine group is the same as the aforementioned examples of the alkyl group. In the present disclosure, the heteroaryl in the heteroarylamine can apply the aforementioned description of the heterocyclic group. In the present disclosure, the alkenyl group in the aralkenyl group is the same as the aforementioned examples of the alkenyl group. In the present disclosure, the aforementioned description of the aryl group can be applied except that the arylene is a divalent group. In the present disclosure, the aforementioned description of the heterocyclic group can be applied except that the heteroarylene is a divalent group. In the present disclosure, the aforementioned description of the aryl group or cycloalkyl group can be applied except that the hydrocarbon ring is not a monovalent group but formed by combining two substituent groups. In the present disclosure, the aforementioned description of the heterocyclic group can be applied, except that the heterocycle is not a monovalent group but formed by combining two substituent groups.

In the present disclosure, provided is a compound of Chemical Formula 1.

The compound of Chemical Formula 1 has a structure in which both a heterocyclic ring containing at least one N and an aromatic fused polycyclic ring having 14 to 60 carbon atoms are bonded to phenylene substituted with a cyano group. In particular, the compound of Chemical Formula 1 can receive electrons from the cathode well and efficiently deliver electrons to the light emitting layer. Thus, it can be effectively applied to an electron injection layer, an electron transport layer, or a hole blocking layer of organic light emitting devices.

In the present disclosure, provided is a compound of Chemical Formula 1:

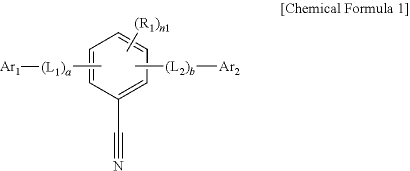

wherein in Chemical Formula 1:
L₁ and L₂ are each independently a substituted or unsubstituted C₆₋₆₀ arylene;

each R₁ is independently a hydrogen, deuterium, halogen, cyano, substituted or unsubstituted C₁₋₆₀ alkyl, substituted or unsubstituted C₁₋₆₀ alkoxy, substituted or unsubstituted C₂₋₆₀ alkenyl, substituted or unsubstituted C₂₋₆₀ alkynyl, substituted or unsubstituted C₃₋₆₀ cycloalkyl, substituted or unsubstituted C₆₋₆₀ aryl, or substituted or unsubstituted C₂₋₆₀ heteroaryl containing any one or more heteroatoms selected from the group consisting of N, O and S;
a and b are each independently 1 or 2;
n1 is an integer from 0 to 3;
one of Ar₁ and Ar₂ is a substituent represented by the following Chemical Formula 2, and the other of Ar₁ and Ar₂ is a substituted or unsubstituted C₁₄₋₆₀ aromatic fused polycyclic ring:

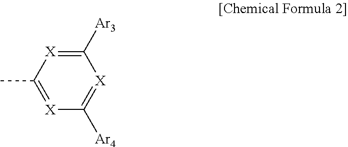

wherein in Chemical Formula 2:
each X is independently N or CH, provided that at least one X is N; and
Ar₃ and Ar₄ are each independently a substituted or unsubstituted C₆₋₆₀ aryl, or substituted or unsubstituted C₂₋₆₀ heteroaryl containing any one or more heteroatoms selected from the group consisting of N, O and S.

In addition, the compound of Chemical Formula 1 can be any one of the following Chemical Formula 1-1 to Chemical Formula 1-3, according to a preferred example of an aromatic fused polycyclic ring as one of Ar₁ and Ar₂:

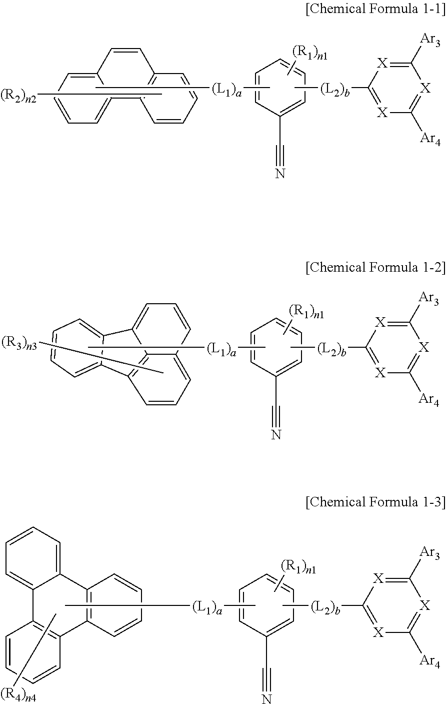

wherein in Chemical Formulae 1-1 to 1-3:
L₁, L₂, a, b, R₁, n1, X, Ar₃, and Ar₄ are as defined in Chemical Formula 1;
R₂, R₃, and R₄ are each independently hydrogen, deuterium, halogen, cyano, substituted or unsubstituted C₁₋₆₀ alkyl, substituted or unsubstituted C₁₋₆₀ alkoxy, substituted or unsubstituted C₂₋₆₀ alkenyl, substituted or unsubstituted C₂₋₆₀ alkynyl, substituted or unsubstituted C₃₋₆₀ cycloalkyl, substituted or unsubstituted C₆₋₆₀ aryl, or substituted or unsubstituted C₂₋₆₀ heteroaryl containing any one or more selected from the group consisting of N, O and S;
n2 is an integer of 0 to 9,
n3 is an integer of 0 to 9, and
n4 is an integer of 0 to 11.

Specifically, in Chemical Formula 1 and Chemical Formulae 1-1 to 1-3, L₁ and L₂ can be each independently a substituted or unsubstituted C₆₋₃₀ arylene, C₆₋₂₈ arylene, C₆₋₂₅ arylene, or C₆₋₂₀ arylene.

Preferably, L₁ and L₂ can be each independently a substituted or unsubstituted C₆₋₂₀ arylene, or an unsubstituted C₆₋₁₈ arylene, or an unsubstituted C₆₋₁₂ arylene. For example, L₁ and L₂ can be each independently phenylene, biphenylylene, or naphthylene.

In particular, L₁ and L₂ can be each independently any one substituent selected from the group consisting of the following substituents:

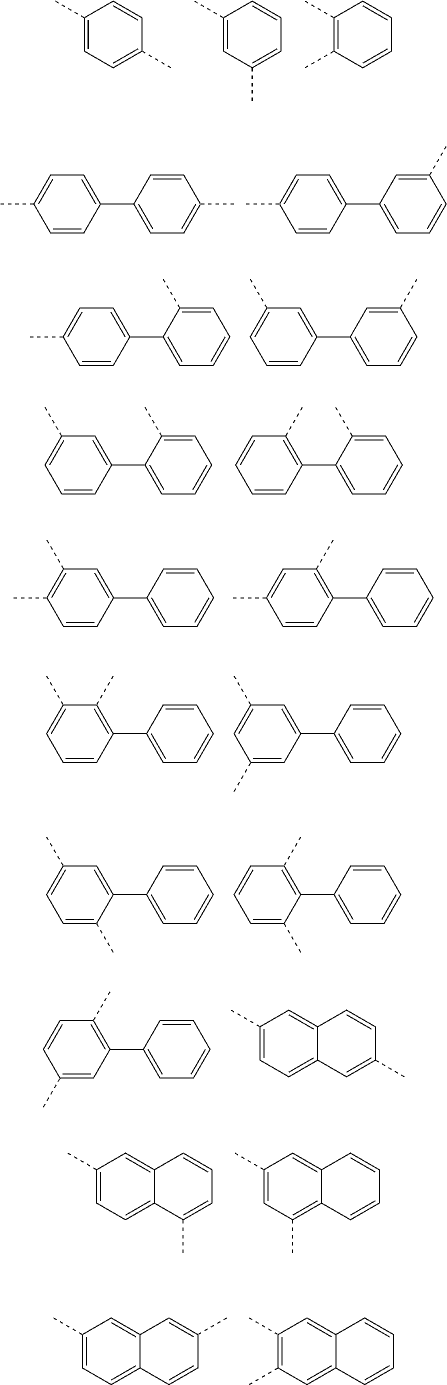

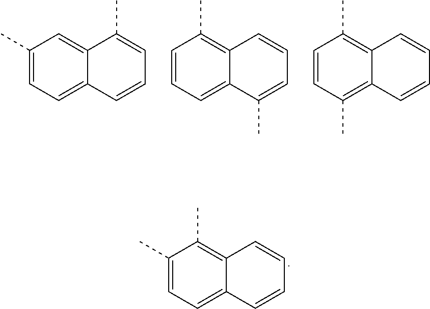

More preferably, at least one of L₁ and L₂ can be phenylene, and the rest of L₁ and L₂ can be phenylene or biphenylylene. For example, at least one of L₁ and L₂ can be phenylene, and the rest of L₁ and L₂ can be biphenylylene, or both of L₁ and L₂ can be phenylene.

Specifically, in Chemical Formula 1 and Chemical Formulae 1-1 to 1-3, a and b can be each independently 1 or 2. In particular, both of a and b can be 1.

Specifically, in Chemical Formula 1 and Chemical Formulae 1-1 to 1-3, each R₁ can independently be hydrogen; deuterium; halogen; cyano; substituted or unsubstituted C₁₋₂₀ alkyl, or C₁₋₁₂ alkyl, or C₁₋₆ alkyl; substituted or unsubstituted C₁₋₂₀ alkoxy, or C₁₋₁₂ alkoxy, or C₁₋₆ alkoxy; substituted or unsubstituted C₂₋₂₀ alkenyl, or C₂₋₁₂ alkenyl, or C₂₋₆ alkenyl; substituted or unsubstituted C₂₋₂₀ alkynyl, or C₂₋₁₂ alkynyl, or C₂₋₆ alkynyl; substituted or unsubstituted C₃₋₃₀ cycloalkyl, or C₃₋₂₅ cycloalkyl, or C₃₋₂₀ cycloalkyl, or C₃₋₁₂ cycloalkyl; substituted or unsubstituted C₆₋₃₀ aryl, or C₆₋₂₈ aryl, or C₆₋₂₅ aryl, or C₆₋₁₈ aryl, or C₆₋₁₂ aryl; or substituted or unsubstituted C₃₋₃₀ heteroaryl, or C₄₋₂₀ heteroaryl, or C₄₋₁₈ heteroaryl, or C₄₋₁₂ heteroaryl, containing any one or more heteroatoms selected from the group consisting of N, O and S.

In particular, each R₁ can independently be hydrogen; deuterium; halogen; cyano; substituted or unsubstituted C₁₋₆ alkyl; substituted or unsubstituted C₁₋₆ alkoxy; substituted or unsubstituted C₂₋₆ alkenyl; substituted or unsubstituted C₂₋₆ alkynyl; substituted or unsubstituted C₃₋₁₂ cycloalkyl; substituted or unsubstituted C₆₋₁₂ aryl; or substituted or unsubstituted C₄₋₁₂ heteroaryl containing any one or more heteroatoms selected from the group consisting of N, O and S.

For example, each R₁ can independently be hydrogen, deuterium, C₁₋₆ alkyl, or C₆₋₁₂ aryl. Preferably, each R₁ can independently be hydrogen or deuterium. More preferably, every R₁ can be hydrogen.

Specifically, n1 can be an integer from 0 to 2, or an integer from 0 to 1. In particular, a structure when n1 is 0, is the same as that when all of R₁ are hydrogen.

In Chemical Formula 1 and Chemical Formulae 1-1 to 1-3, one of Ar₁ and Ar₂ is a substituent represented by Chemical Formula 2, and the other of Ar₁ and Ar₂ is substituted or unsubstituted C₁₄₋₆₀ aromatic fused polycyclic ring, preferably a substituted or unsubstituted C₁₄₋₆₀ aromatic fused polycyclic ring having at least two aryl groups of C₆₋₂₀ in a fused form. Herein, a C₁₄₋₆₀ aromatic fused polycyclic ring refers to a polycyclic ring having a carbon number of 14 to 60, which is a type of polynuclear aromatic hydrocarbons with a covalent pair of carbon atoms shared by two of the carbon atoms of the benzene ring. For example, the C₁₄₋₆₀ aromatic fused polycyclic ring can be an aryl polynuclear fused ring having a carbon number of 14 to 60, wherein at least two aryl groups of C₆₋₂₀ are fused with a C₆₋₂₀ arylene or C₄₋₂₀ alkylene. Specifically, the C₁₄₋₆₀ aromatic fused polycyclic ring can be an aryl polynuclear fused ring having a carbon number of 14 to 60 in which at least two phenyls, preferably, at least two selected from phenyl and naphthyl, are fused with a C₆₋₂₀ arylene or C₄₋₂₀ alkylene.

In the present disclosure, as described above, the compound of Chemical Formula 1 has a structure in which one of Ar₁ and Ar₂ is substituted or unsubstituted C₁₄₋₆₀ aromatic fused polycyclic ring, preferably, substituted or unsubstituted C₁₄₋₆₀ aromatic fused polycyclic ring having at least two aryl groups of C₆₋₂₀ in a fused form, e.g., substituted or unsubstituted C₁₄₋₆₀ aromatic fused polycyclic ring in which at least two benzene rings are in a fused form. Thus, it is possible to improve characteristics of the organic light emitting device, such as the characteristics of driving voltage, luminescence efficiency, and lifetime of the organic light-emitting device. In particular, the compound of Chemical Formula 1 can receive electrons from the cathode well and efficiently deliver electrons to the light emitting layer. Therefore, it is advantageous in terms of improving characteristics such as driving voltage, luminescence efficiency, and lifetime of the organic light-emitting device.

Preferably, one of Ar₁ and Ar₂ is a substituent represented by Chemical Formula 2, and the other of Ar₁ and Ar₂ can be substituted or unsubstituted phenanthrenyl, or substituted or unsubstituted triphenylenyl, or substituted or unsubstituted fluoranthenyl.

For example, one of Ar₁ and Ar₂ is a substituent represented by Chemical Formula 2, and the other of Ar₁ and Ar₂ can be any one selected from the group consisting of the following substituents:

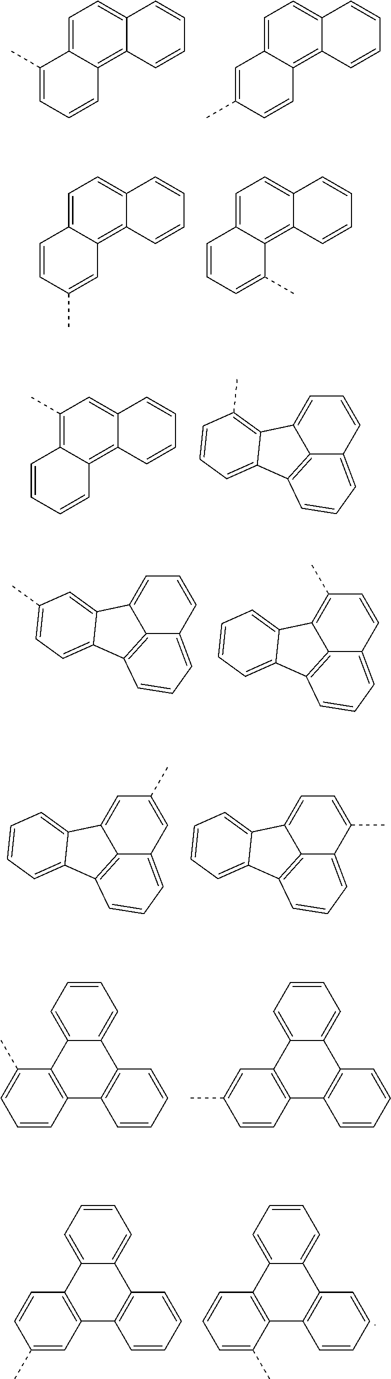

Further, in Chemical Formula 1 and Chemical Formulae 1-1 to 1-3, one of Ar₁ and Ar₂ can be a substituted or unsubstituted C₁₄₋₆₀ aromatic fused polycyclic ring, and the other of Ar₁ and Ar₂ can be a substituent represented by Chemical Formula 2.

Specifically, in Chemical Formula 1 and Chemical Formulae 1-1 to 1-3, one or two of the Xs can be N, and the remaining Xs can be CH. Alternatively, all of the Xs can be N.

In Chemical Formula 1 and Chemical Formulae 1-1 to 1-3, Ar₃ and Ara can independently be a substituted or unsubstituted C₆₋₃₀ aryl, or a C₆₋₂₈ aryl, or a C₆₋₂₅ aryl, or a substituted or unsubstituted C₅₋₃₀ heteroaryl, a C₈₋₂₀ heteroaryl, or a C₁₂₋₁₈ heteroaryl containing one or more heteroatoms selected from the group consisting of N, O and S.

Preferably, Ar₃ and Ara can independently be a substituted or unsubstituted C₆₋₂₅ aryl, or a substituted or unsubstituted C₁₂₋₁₈ heteroaryl containing one or more heteroatoms selected from the group consisting of N, O and S. More preferably, Ar₃ and Ara can independently be a substituted or unsubstituted C₆₋₂₅ aryl.

In addition, Ar₃ and Ara can independently be phenyl; phenyl substituted with one to five deuteriums; phenyl substituted with one to five methyls; phenyl substituted with one to five cyanyls; phenyl substituted with naphthyl; phenyl substituted with phenathrenyl; phenyl substituted with triphenylenyl; phenyl substituted with fluoranthenyl; biphenyl; biphenyl substituted with one to nine deuteriums; biphenyl substituted with one to nine methyls; biphenyl substituted with one to nine cyanyls; terphenyl; quaterphenyl; naphthyl; naphthyl substituted with phenyl; phenanthrenyl; triphenylenyl; or fluoranthenyl.

For example, Ar₃ and Ara can independently be any one substituent selected from the group consisting of the following substituents:

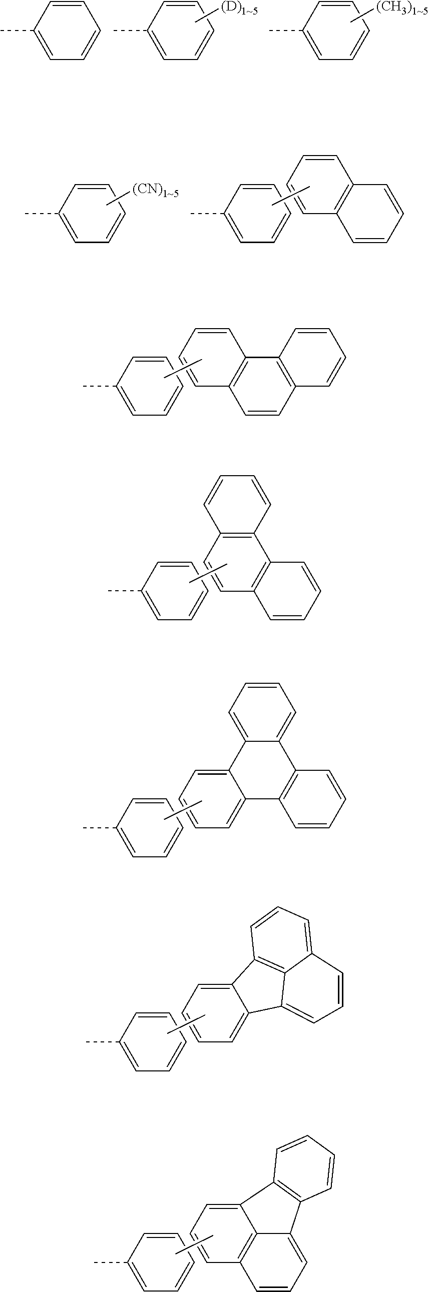

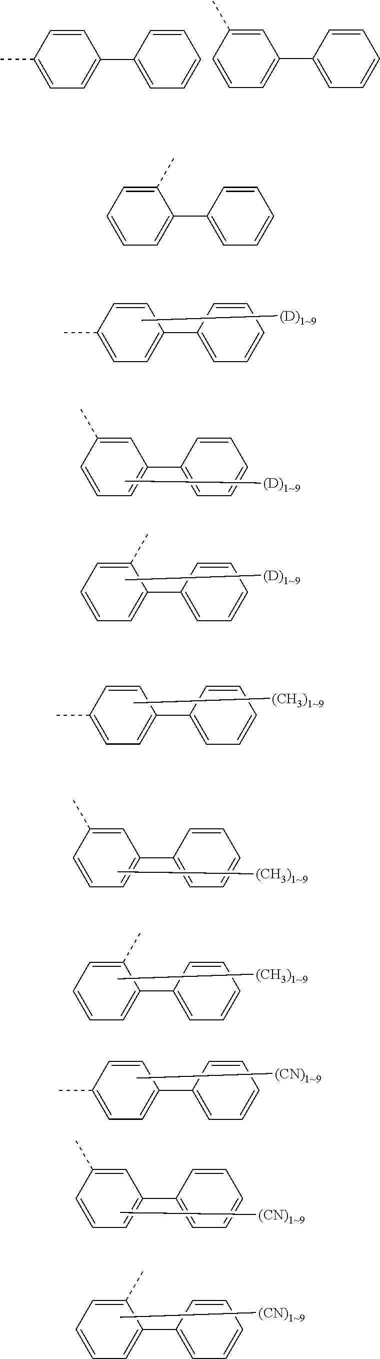

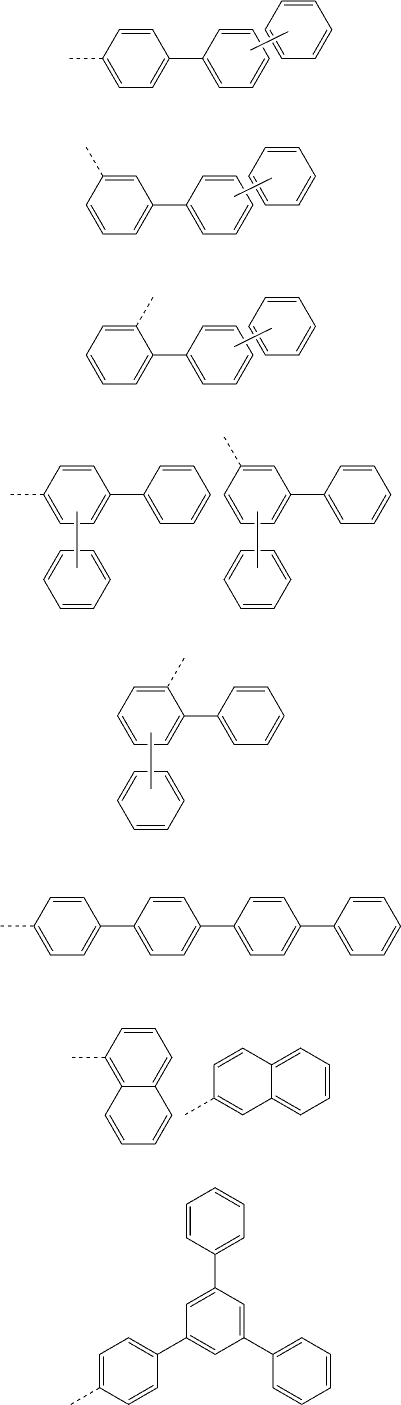

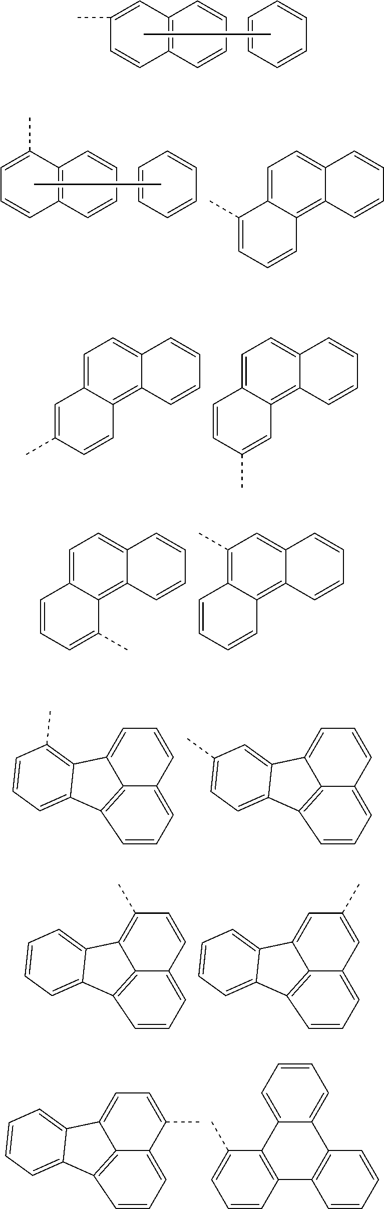

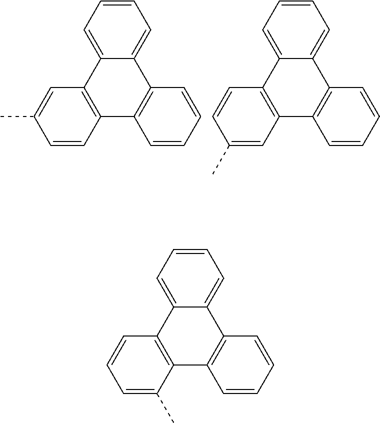

wherein in the above formulae, D is deuterium, CH₃ is methyl, and ON is cyanyl.

In particular, Ar₃ and Ar₄ can independently be phenyl; phenyl substituted with five deuteriums; phenyl substituted with one or two methyls; phenyl substituted with one or two cyanyls; phenyl substituted with naphthyl; phenyl substituted with phenanthrenyl; phenyl substituted with triphenylenyl; phenyl substituted with fluoranthenyl; biphenyl; biphenyl substituted with five or nine deuteriums; biphenyl substituted with one or two methyls; biphenyl substituted with one or two cyanyls; terphenyl; quaterphenyl; naphthyl; naphthyl substituted with phenyl; phenanthrenyl; triphenylenyl; or fluoranthenyl.

For example, Ar₃ and Ar₄ can independently be phenyl; phenyl substituted with one or two methyls; phenyl substituted with one cyanyl; phenyl substituted with naphthyl; phenyl substituted with phenanthrenyl; biphenyl; biphenyl substituted with one cyanyl; phenanthrenyl; or fluoranthenyl.

Specifically, in Chemical Formulae 1-1 to 1-3, R₂, R₃, and R₄ can independently be hydrogen; deuterium; halogen; cyano; substituted or unsubstituted C₁₋₂₀ alkyl, or C₁₋₁₂ alkyl, or C₁₋₆ alkyl; substituted or unsubstituted C₁₋₂₀ alkoxy, or C₁₋₁₂ alkoxy, or C₁₋₆ alkoxy; substituted or unsubstituted C₂₋₂₀ alkenyl, or C₂₋₁₂ alkenyl, or C₂₋₆ alkenyl; substituted or unsubstituted C₂₋₂₀ alkynyl, or C₂₋₁₂ alkynyl, or C₂₋₆ alkynyl; substituted or unsubstituted C₃₋₃₀ cycloalkyl, or C₃₋₂₅ cycloalkyl, or C₃₋₂₀ cycloalkyl, or C₃₋₁₂ cycloalkyl; substituted or unsubstituted C₆₋₃₀ aryl, or C₆₋₂₈ aryl, or C₆₋₂₅ aryl, or C₆₋₁₈ aryl, or C₆₋₁₂ aryl; or substituted or unsubstituted C₃₋₃₀ heteroaryl, or C₄₋₂₀ heteroaryl, or C₄₋₁₈ heteroaryl, or C₄₋₁₂ heteroaryl, containing any one or more selected from the group consisting of N, O and S.

In particular, R₂, R₃, and R₄ can independently be hydrogen; deuterium; halogen; cyano; substituted or unsubstituted C₁₋₆ alkyl; substituted or unsubstituted C₁₋₆ alkoxy; substituted or unsubstituted C₂₋₆ alkenyl; substituted or unsubstituted C₂₋₆ alkynyl; substituted or unsubstituted C₃₋₁₂ cycloalkyl; substituted or unsubstituted C₆₋₁₂ aryl; or substituted or unsubstituted C₄₋₁₂ heteroaryl containing any one or more selected from the group consisting of N, O and S.

For example, R₂, R₃, and R₄ can independently be hydrogen, deuterium, C₁₋₆ alkyl, or C₆₋₁₂ aryl. Preferably, R₂, R₃, and R₄ can independently be hydrogen, or deuterium. More preferably, all of R₂, R₃, and R₄ can be hydrogen.

Specifically, n2 and n3 can independently be an integer from 0 to 8, or an integer from 0 to 6, or an integer from 0 to 5, or an integer from 0 to 2, or n2 and n3 can independently be 0 or 1. In particular, a structure when n2 is 0, is the same as that when all of R₂ are hydrogen. Further, a structure when n3 is 0, is the same as that when all of R₃ are hydrogen.

In addition, n4 can be an integer from 0 to 10, an integer from 0 to 8, or an integer from 0 to 6, or an integer from 0 to 5, or an integer from 0 to 2, or n4 can be 0 or 1. In particular, a structure when n4 is 0, is the same as that when all of R₄ are hydrogen.

Meanwhile, any hydrogen of the compound of Chemical Formula 1 can be substituted with deuterium.

Representative examples of the compound of Chemical Formula 1 are as follows.

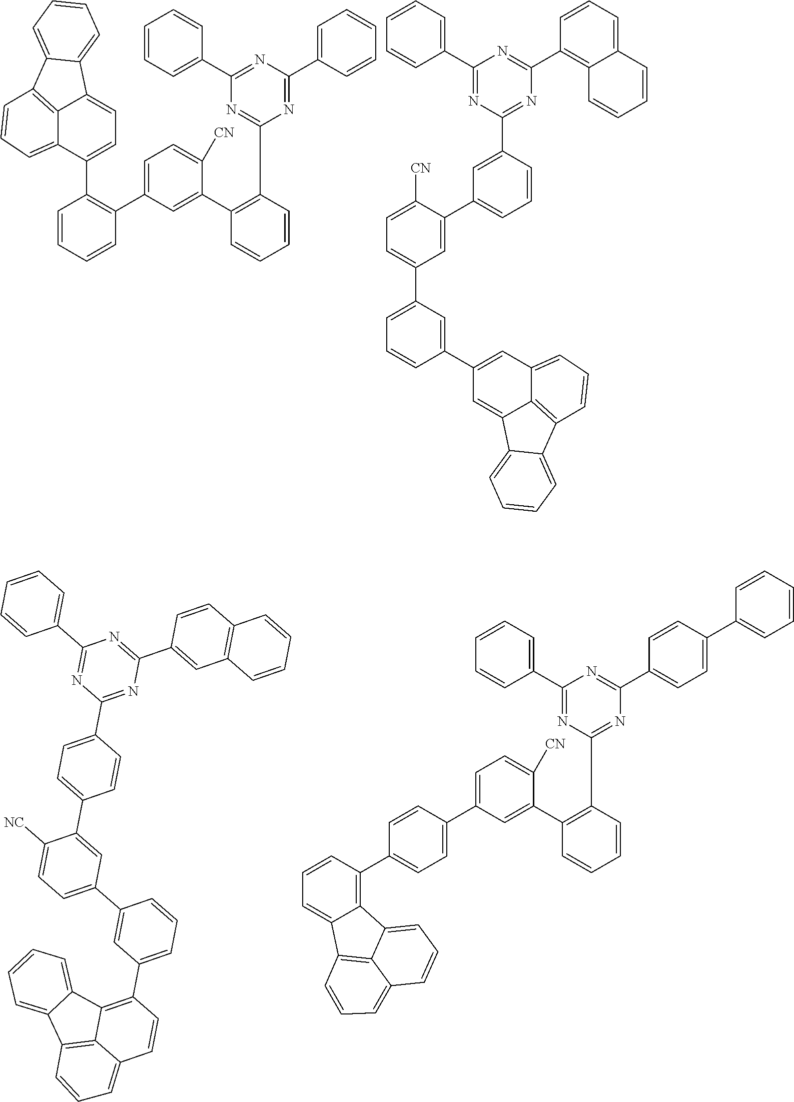

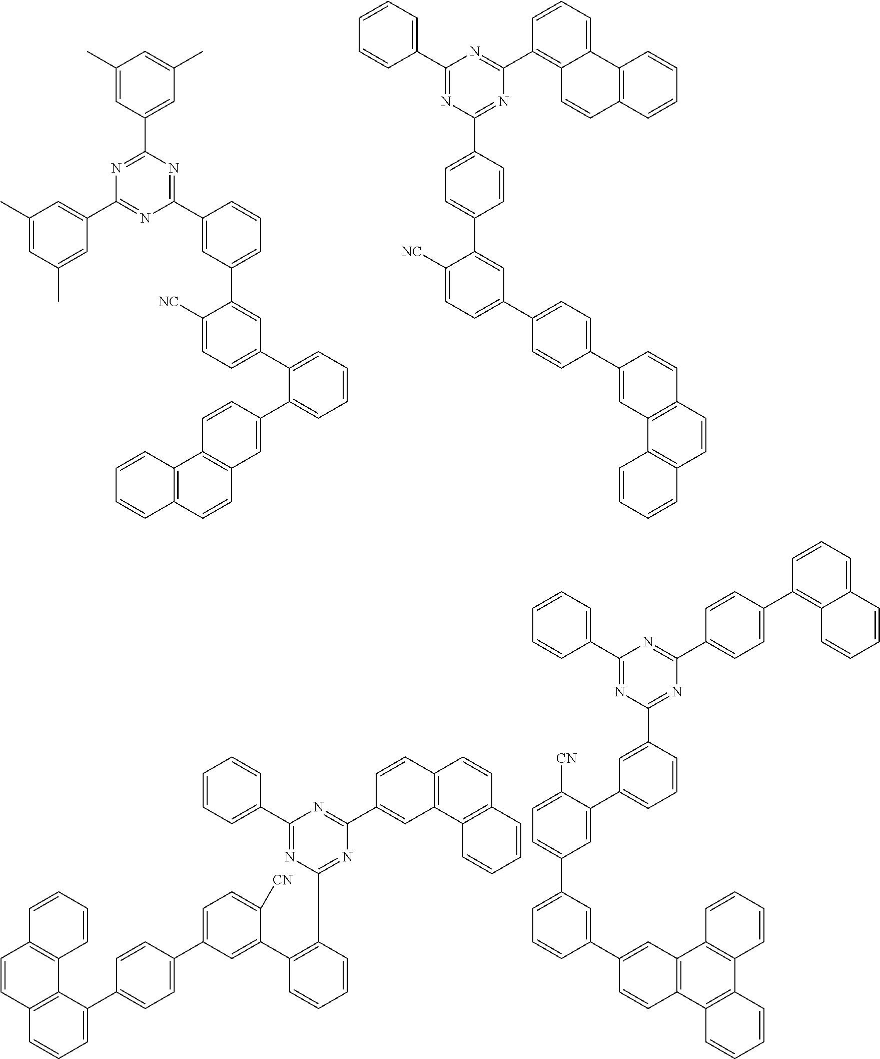

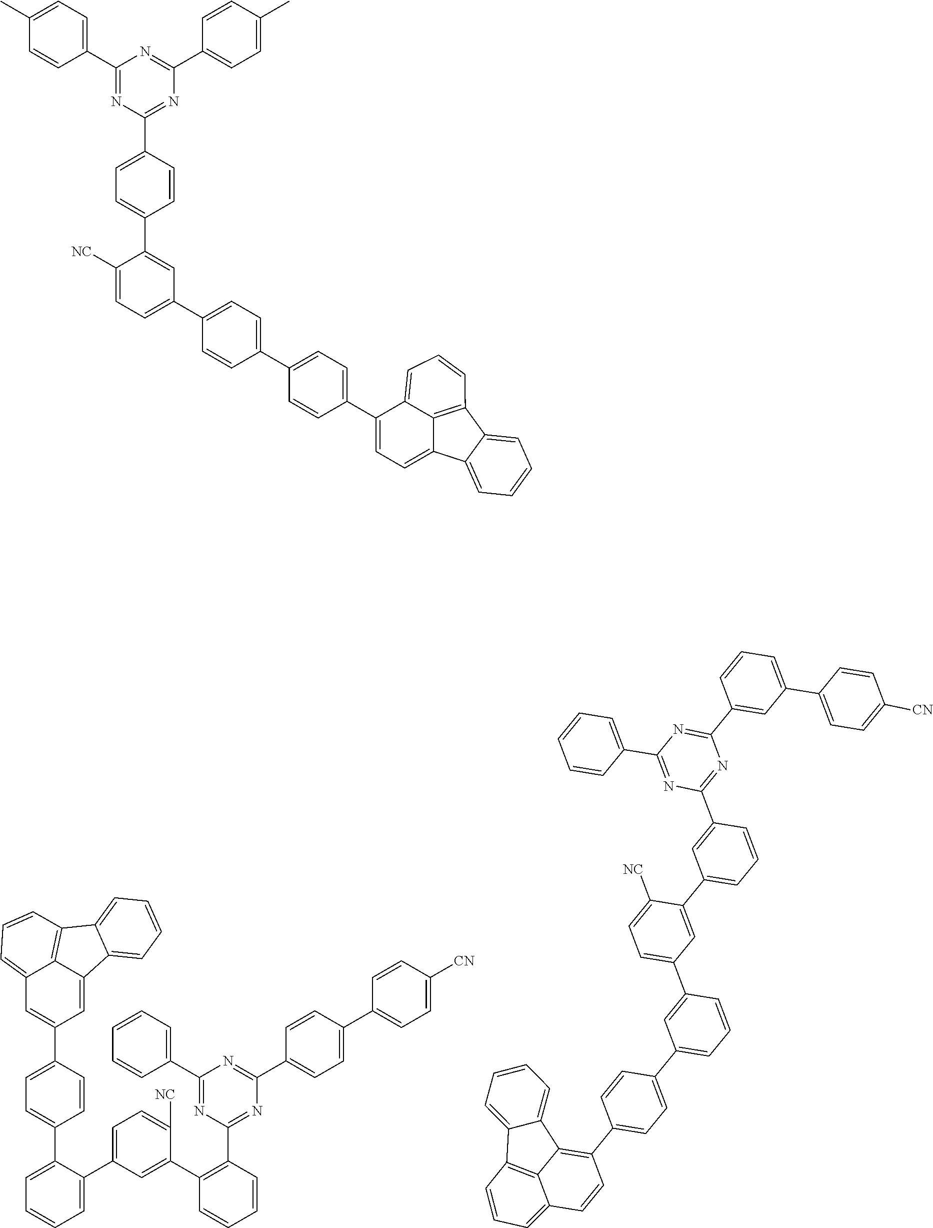 [SMILES: Cc1ccc(-c2nc(-c3ccc(C)cc3)nc(-c3ccc(-c4cc(-c5ccc(-c6ccc(-c7ccc8c9c(cccc79)-c7ccccc7-8)cc6)cc5)ccc4C#N)cc3)n2)cc1 ; N#Cc1ccc(-c2ccc(-c3nc(-c4ccccc4)nc(-c4ccccc4-c4cc(-c5ccccc5-c5ccc(-c6cc7c8c(cccc8c6)-c6ccccc6-7)cc5)ccc4C#N)n3)cc2)cc1 ; N#Cc1ccc(-c2cccc(-c3nc(-c4ccccc4)nc(-c4cccc(-c5cc(-c6cccc(-c7ccc(-c8ccc9cccc%10c9c8-c8ccccc8-%10)cc7)c6)ccc5C#N)c4)n3)c2)cc1]

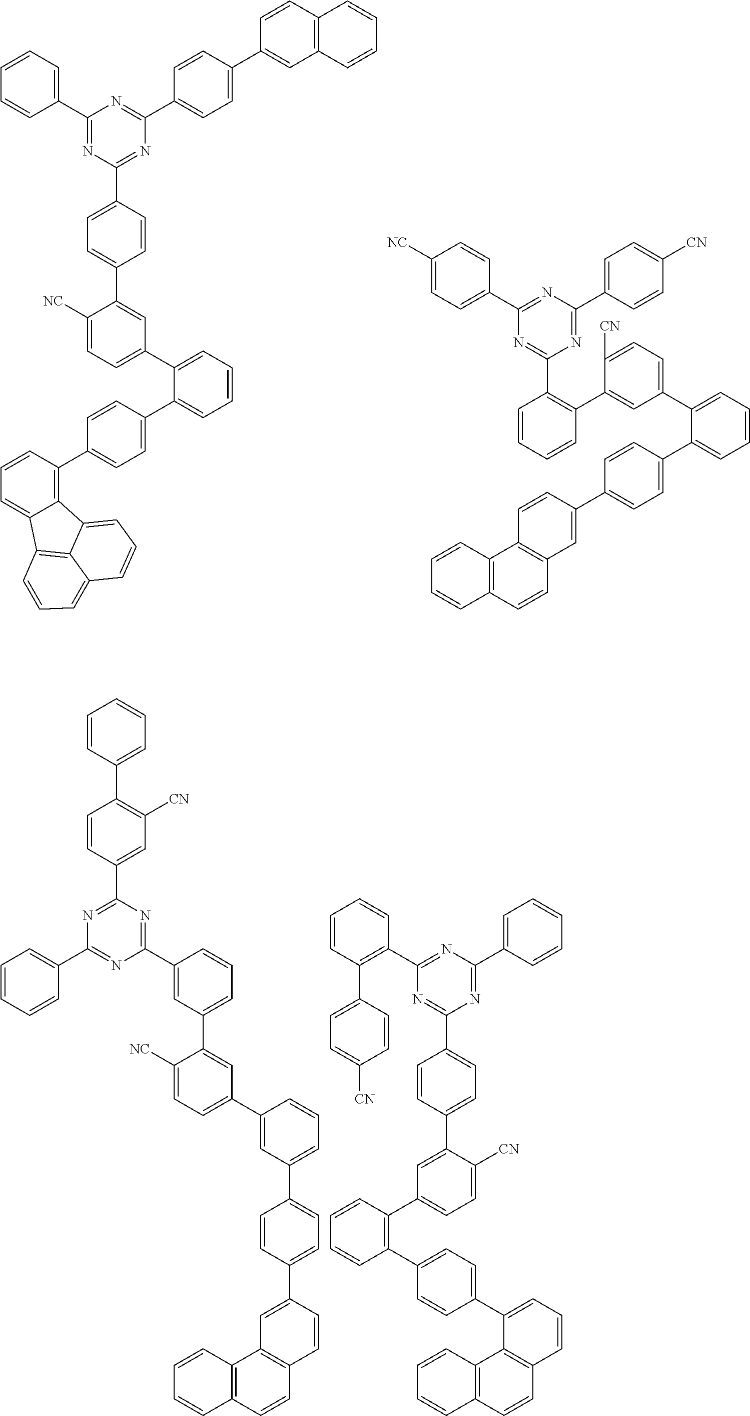

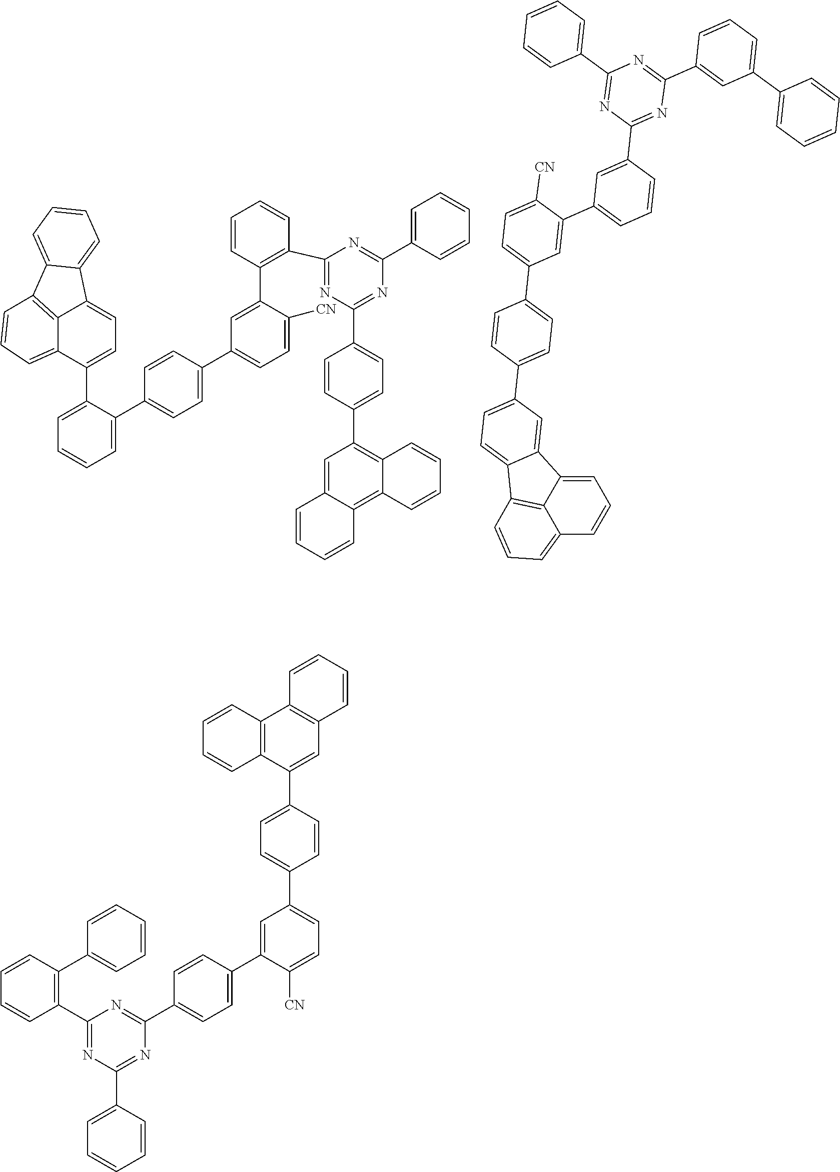

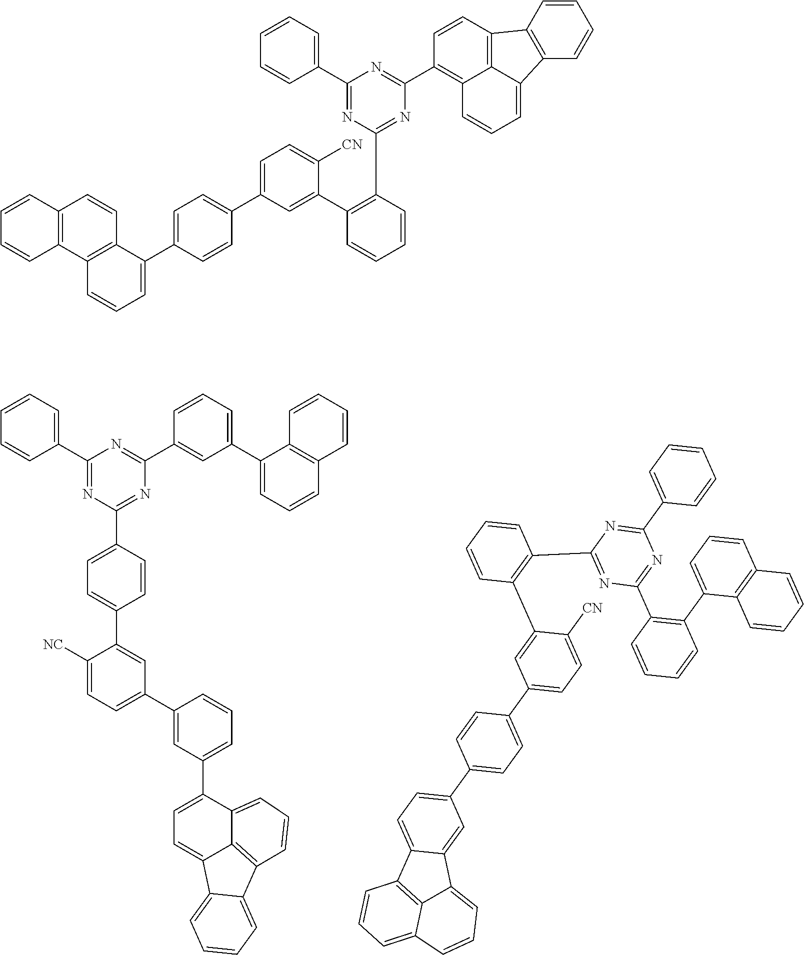 [SMILES: N#Cc1ccc(-c2ccc(-c3cccc4c3ccc3ccccc34)cc2)cc1-c1ccccc1-c1nc(-c2ccccc2)nc(-c2ccc3c4c(cccc24)-c2ccccc2-3)n1 ; N#Cc1ccc(-c2cccc(-c3ccc4c5c(cccc35)-c3ccccc3-4)c2)cc1-c1ccc(-c2nc(-c3ccccc3)nc(-c3cccc(-c4cccc5ccccc45)c3)n2)cc1 ; N#Cc1ccc(-c2ccc(-c3ccc4c(c3)-c3cccc5cccc-4c35)cc2)cc1-c1ccccc1-c1nc(-c2ccccc2)nc(-c2ccccc2-c2cccc3ccccc23)n1]

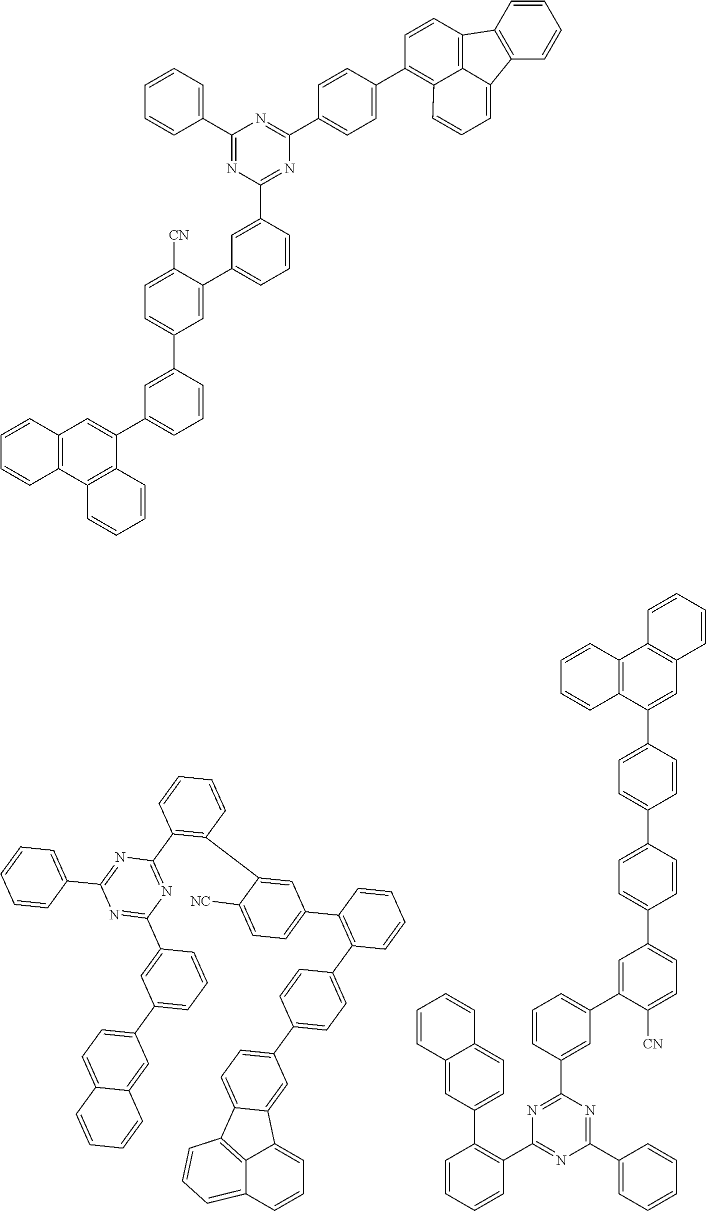 [SMILES: N#Cc1ccc(-c2cccc(-c3cc4ccccc4c4ccccc34)c2)cc1-c1cccc(-c2nc(-c3ccccc3)nc(-c3ccc(-c4ccc5c6c(cccc46)-c4ccccc4-5)cc3)n2)c1 ; N#Cc1ccc(-c2ccccc2-c2ccc(-c3ccc4c(c3)-c3cccc5cccc-4c35)cc2)cc1-c1ccccc1-c1nc(-c2ccccc2)nc(-c2cccc(-c3ccc4ccccc4c3)c2)n1 ; N#Cc1ccc(-c2ccc(-c3ccc(-c4cc5ccccc5c5ccccc45)cc3)cc2)cc1-c1cccc(-c2nc(-c3ccccc3)nc(-c3ccccc3-c3ccc4ccccc4c3)n2)c1]

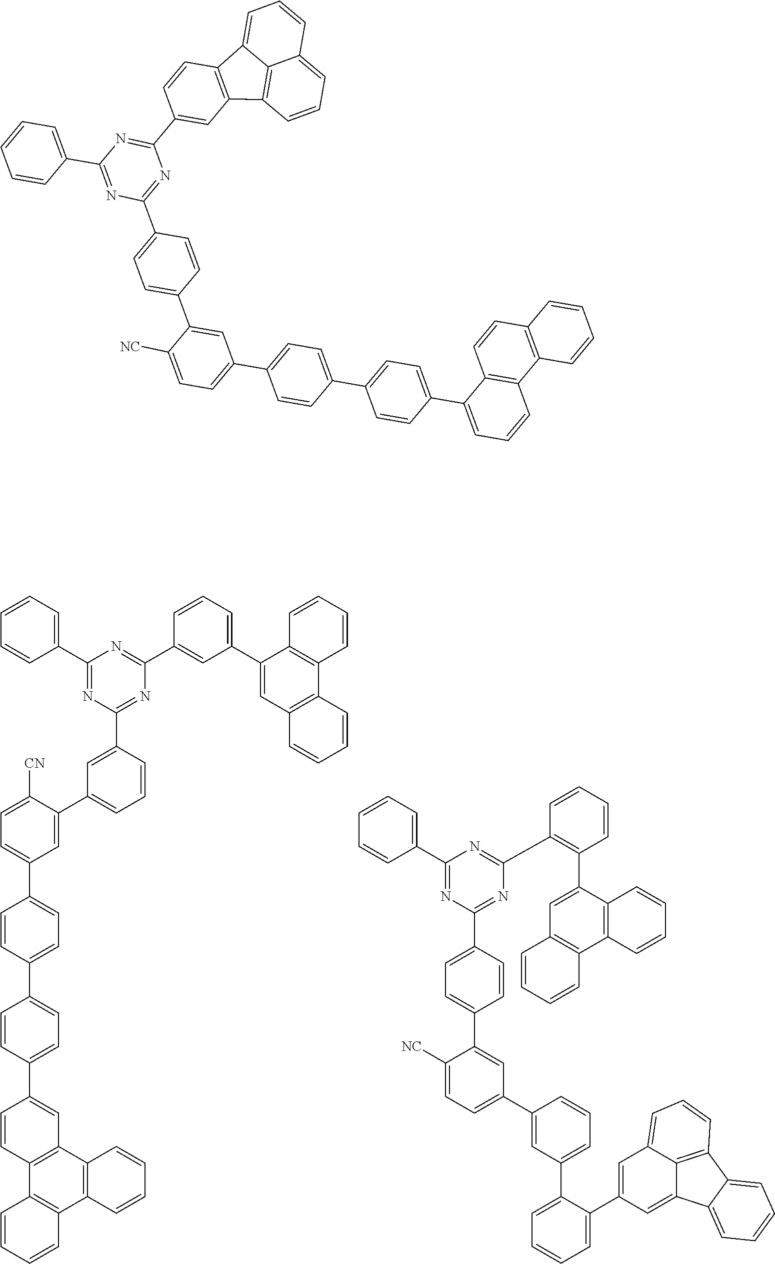 [SMILES: N#Cc1ccc(-c2ccc(-c3ccc(-c4cccc5c4ccc4ccccc45)cc3)cc2)cc1-c1ccc(-c2nc(-c3ccccc3)nc(-c3ccc4c(c3)-c3cccc5cccc-4c35)n2)cc1 ; N#Cc1ccc(-c2ccc(-c3ccc(-c4ccc5c6ccccc6c6ccccc6c5c4)cc3)cc2)cc1-c1cccc(-c2nc(-c3ccccc3)nc(-c3cccc(-c4cc5ccccc5c5ccccc45)c3)n2)c1 ; N#Cc1ccc(-c2cccc(-c3ccccc3-c3cc4c5c(cccc5c3)-c3ccccc3-4)c2)cc1-c1ccc(-c2nc(-c3ccccc3)nc(-c3ccccc3-c3cc4ccccc4c4ccccc34)n2)cc1]

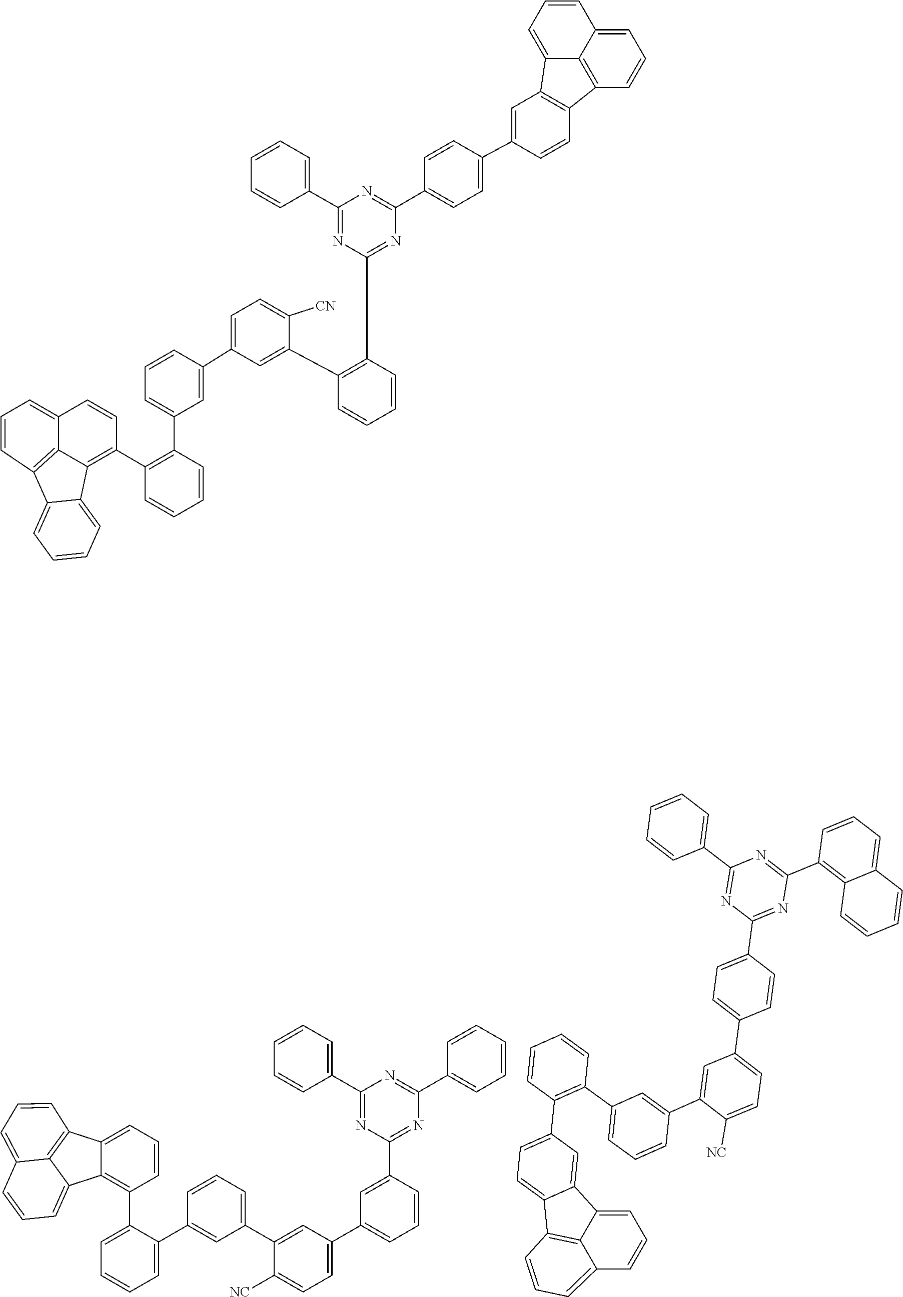

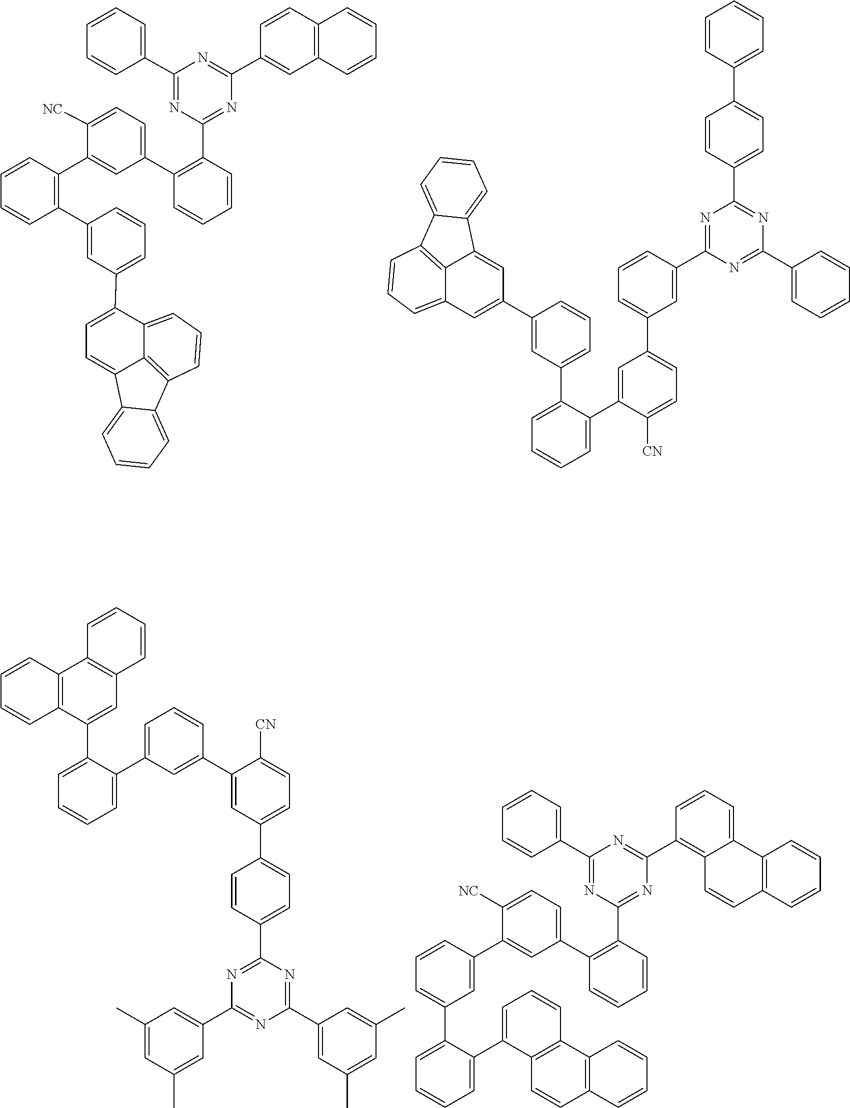

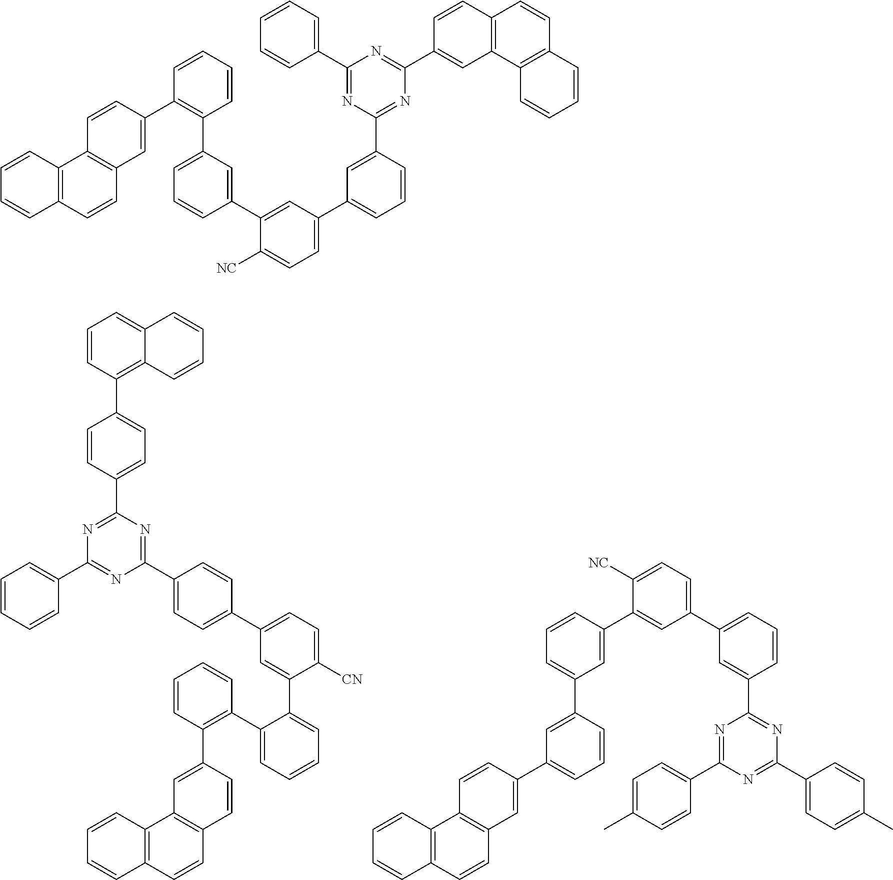 [SMILES: N#Cc1ccc(-c2cccc(-c3nc(-c4ccccc4)nc(-c4ccc5ccc6ccccc6c5c4)n3)c2)cc1-c1cccc(-c2ccccc2-c2ccc3c(ccc4ccccc43)c2)c1 ; N#Cc1ccc(-c2ccc(-c3nc(-c4ccccc4)nc(-c4ccc(-c5cccc6ccccc56)cc4)n3)cc2)cc1-c1ccccc1-c1ccccc1-c1ccc2ccc3ccccc3c2c1 ; Cc1ccc(-c2nc(-c3ccc(C)cc3)nc(-c3cccc(-c4ccc(C#N)c(-c5cccc(-c6cccc(-c7ccc8c(ccc9ccccc98)c7)c6)c5)c4)c3)n2)cc1]

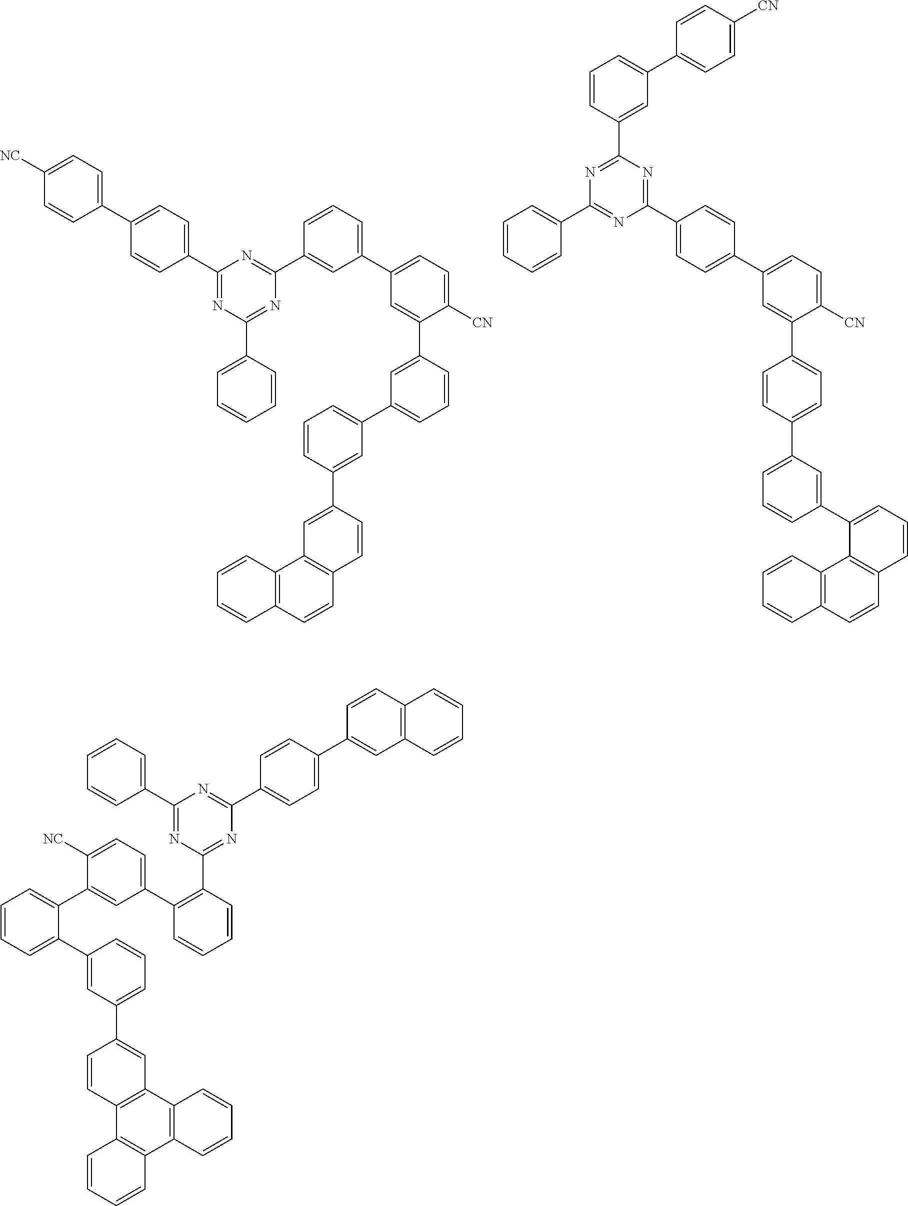 [SMILES: N#Cc1ccc(-c2ccc(-c3nc(-c4ccccc4)nc(-c4cccc(-c5ccc(C#N)c(-c6cccc(-c7cccc(-c8ccc9ccc%10ccccc%10c9c8)c7)c6)c5)c4)n3)cc2)cc1 ; N#Cc1ccc(-c2cccc(-c3nc(-c4ccccc4)nc(-c4ccc(-c5ccc(C#N)c(-c6ccc(-c7cccc(-c8cccc9ccc%10ccccc%10c89)c7)cc6)c5)cc4)n3)c2)cc1 ; N#Cc1ccc(-c2ccccc2-c2nc(-c3ccccc3)nc(-c3ccc(-c4ccc5ccccc5c4)cc3)n2)cc1-c1ccccc1-c1cccc(-c2ccc3c4ccccc4c4ccccc4c3c2)c1]

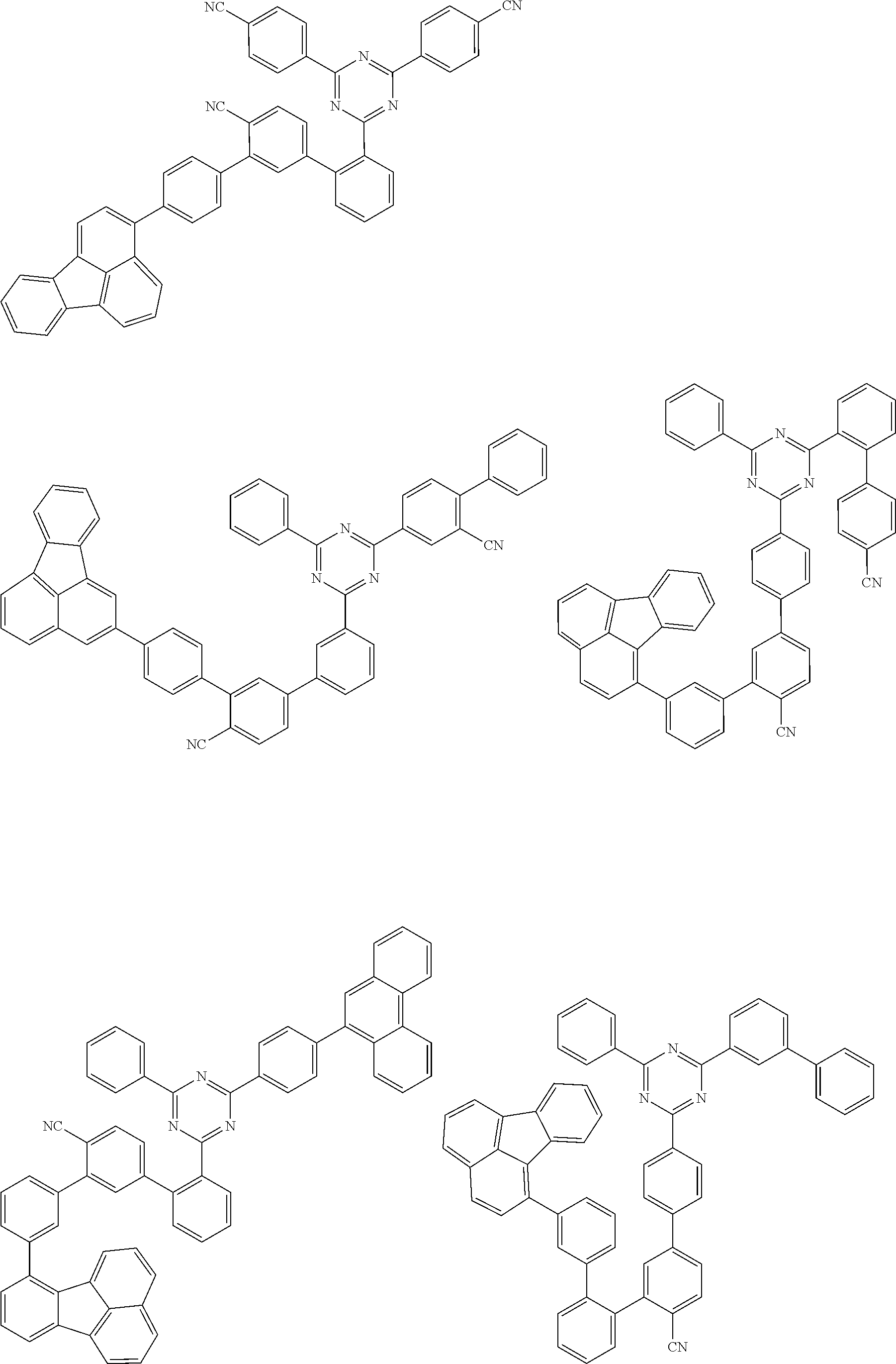

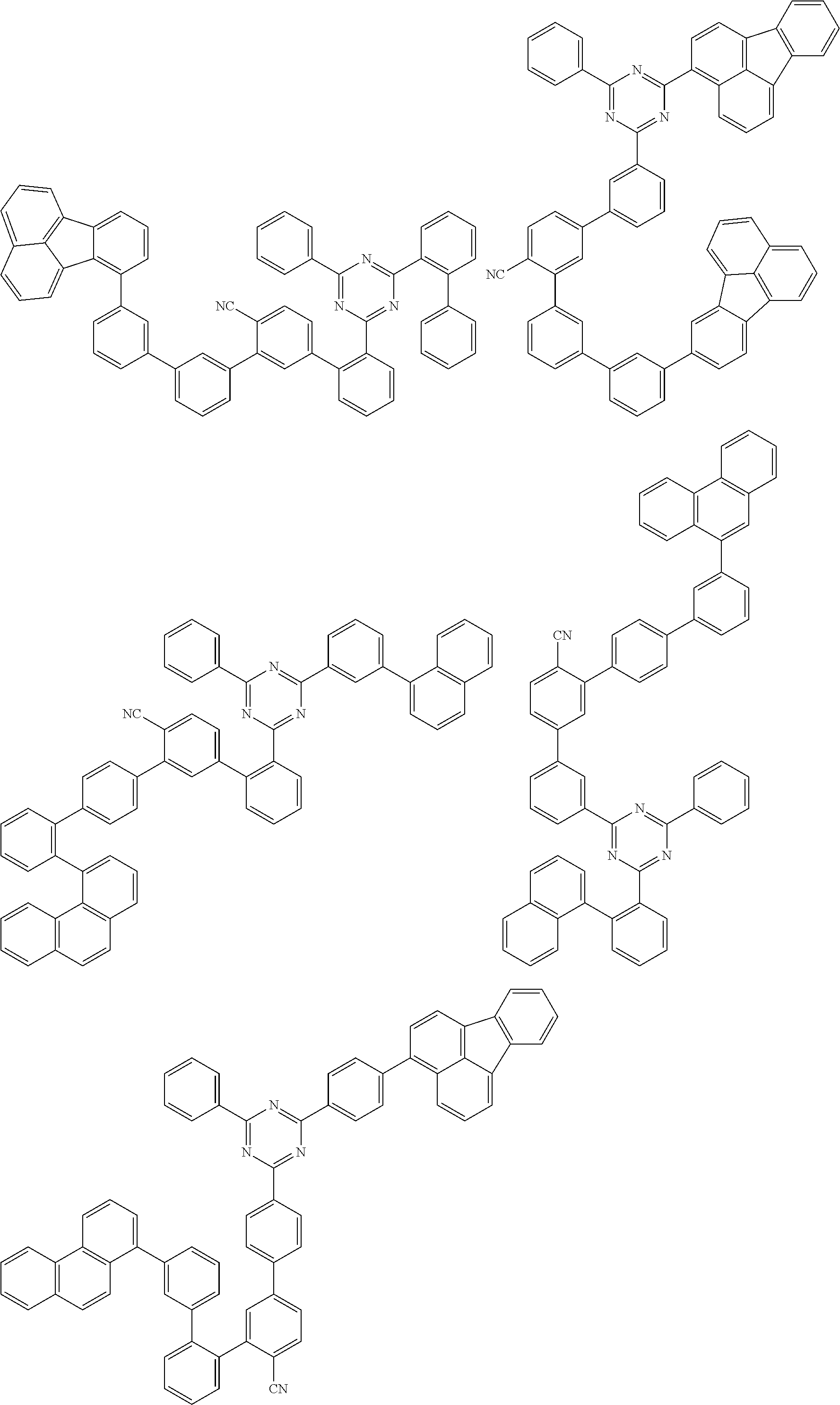

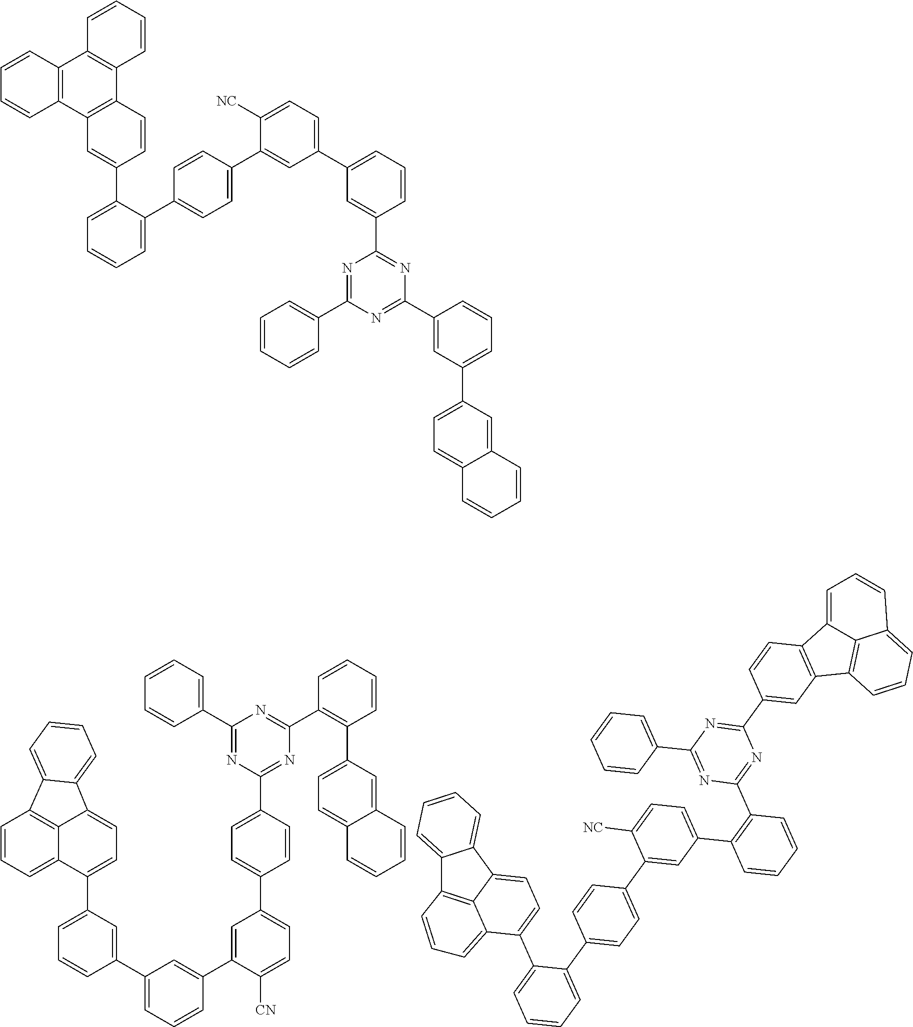

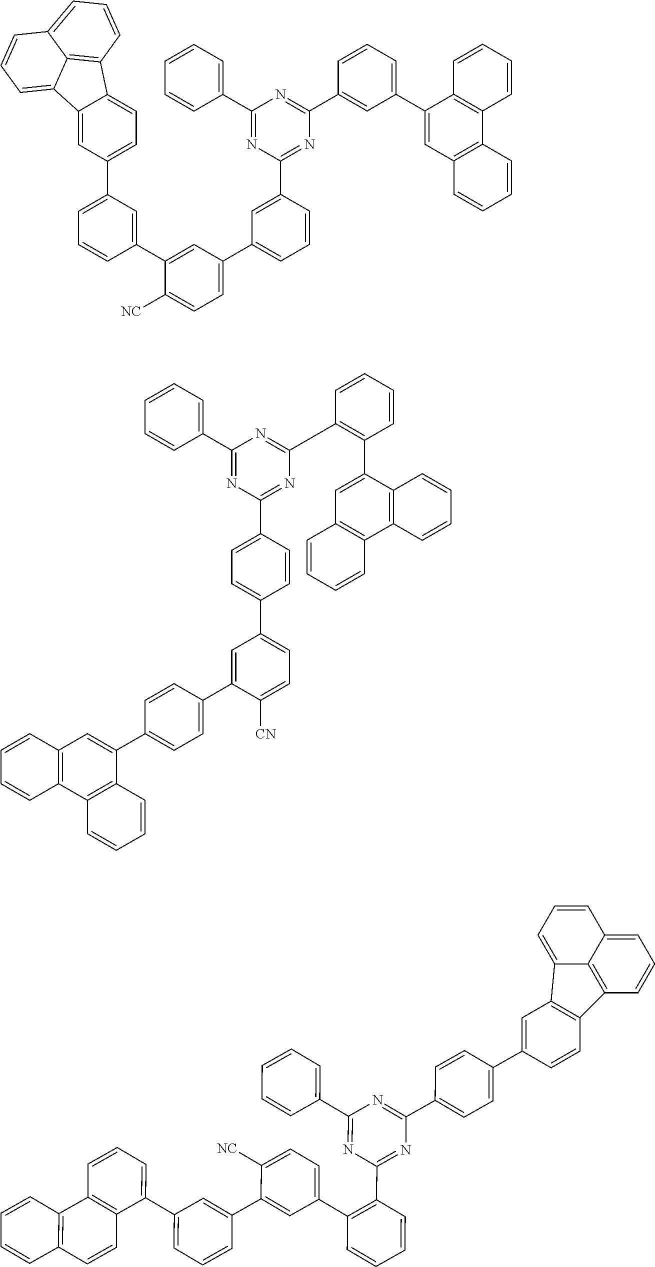 [SMILES: N#Cc1ccc(-c2cccc(-c3nc(-c4ccccc4)nc(-c4cccc(-c5cc6ccccc6c6ccccc56)c4)n3)c2)cc1-c1cccc(-c2ccc3c(c2)-c2cccc4cccc-3c24)c1 ; N#Cc1ccc(-c2ccc(-c3nc(-c4ccccc4)nc(-c4ccccc4-c4cc5ccccc5c5ccccc45)n3)cc2)cc1-c1ccc(-c2cc3ccccc3c3ccccc23)cc1 ; N#Cc1ccc(-c2ccccc2-c2nc(-c3ccccc3)nc(-c3ccc(-c4ccc5c(c4)-c4cccc6cccc-5c46)cc3)n2)cc1-c1cccc(-c2cccc3c2ccc2ccccc23)c1]

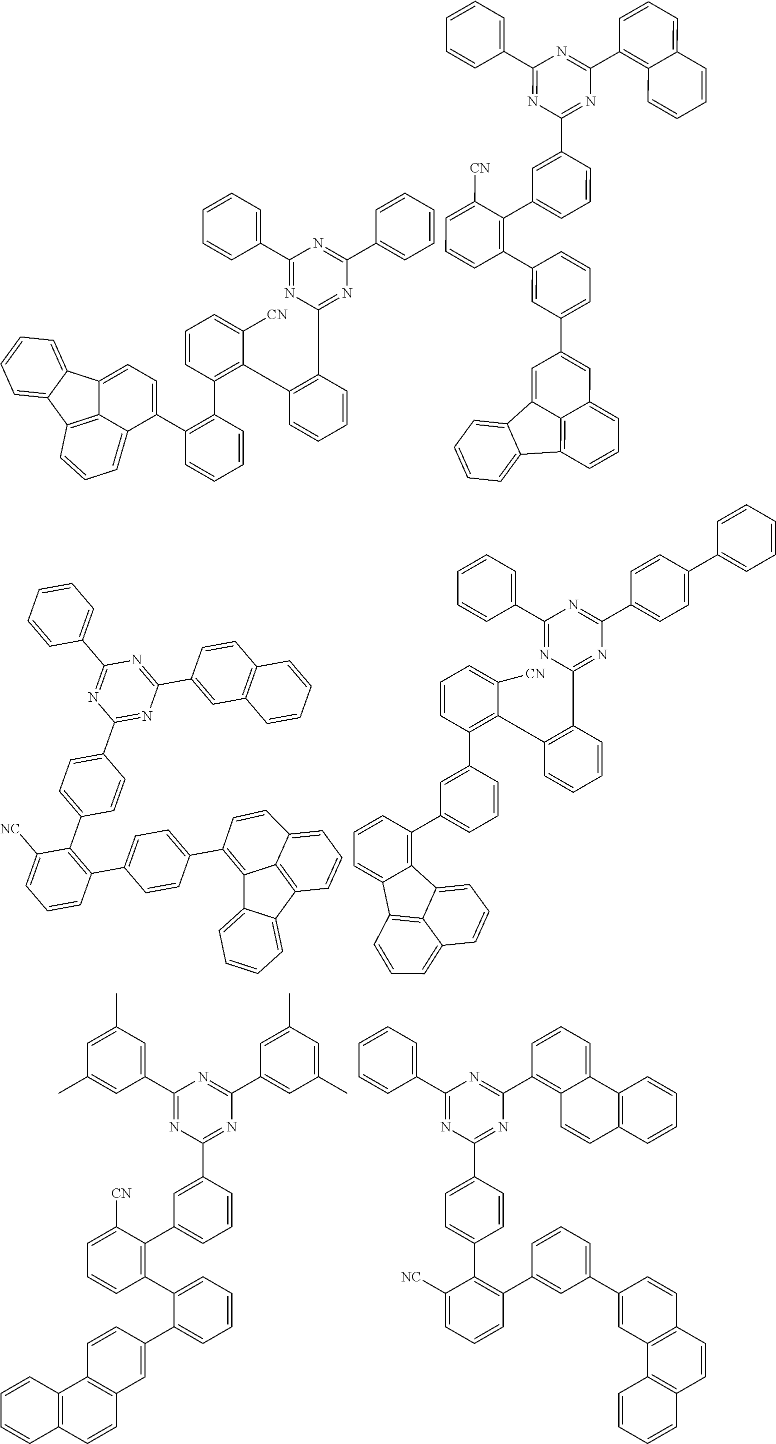

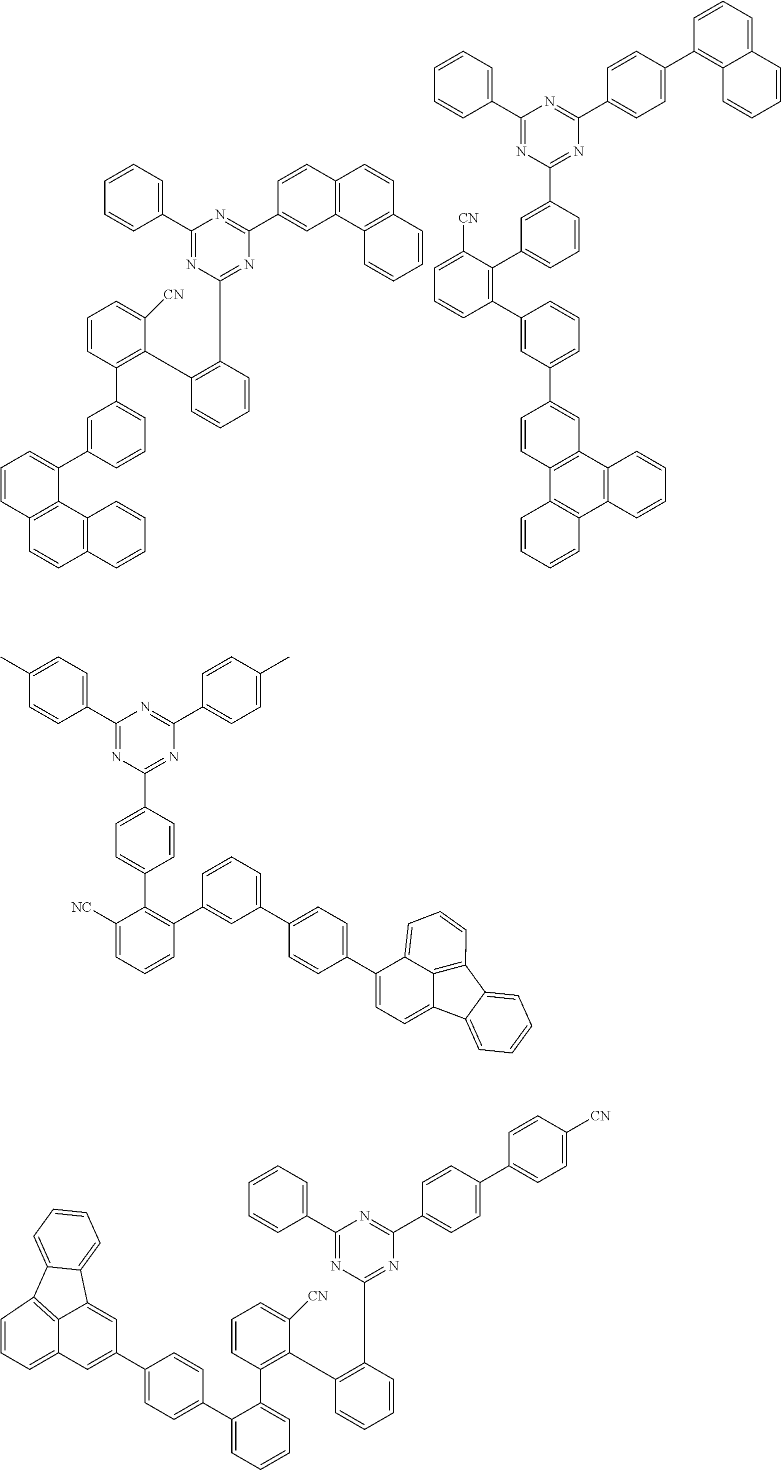

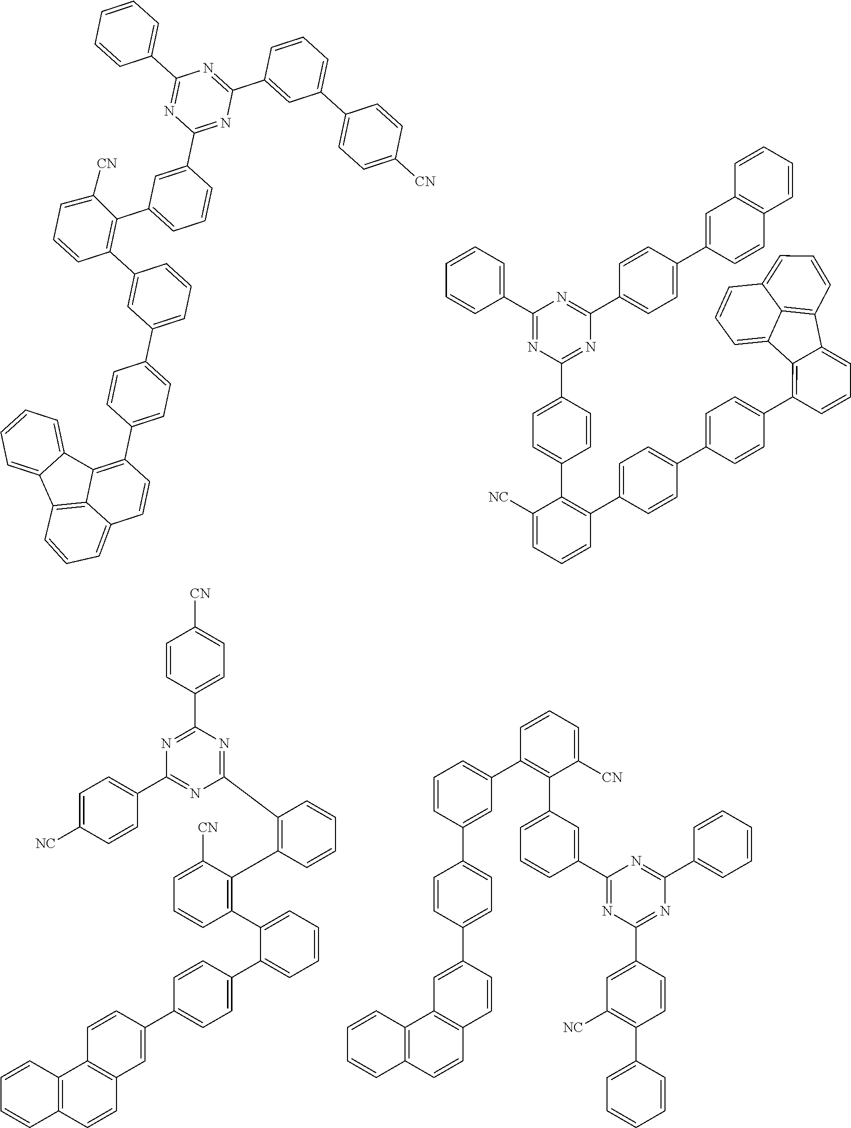 [SMILES: N#Cc1ccc(-c2cccc(-c3nc(-c4ccccc4)nc(-c4cccc(-c5c(C#N)cccc5-c5cccc(-c6ccc(-c7ccc8cccc9c8c7-c7ccccc7-9)cc6)c5)c4)n3)c2)cc1 ; N#Cc1cccc(-c2ccc(-c3ccc(-c4cccc5c4-c4cccc6cccc-5c46)cc3)cc2)c1-c1ccc(-c2nc(-c3ccccc3)nc(-c3ccc(-c4ccc5ccccc5c4)cc3)n2)cc1 ; N#Cc1ccc(-c2nc(-c3ccc(C#N)cc3)nc(-c3ccccc3-c3c(C#N)cccc3-c3ccccc3-c3ccc(-c4ccc5c(ccc6ccccc65)c4)cc3)n2)cc1 ; N#Cc1cc(-c2nc(-c3ccccc3)nc(-c3cccc(-c4c(C#N)cccc4-c4cccc(-c5ccc(-c6ccc7ccc8ccccc8c7c6)cc5)c4)c3)n2)ccc1-c1ccccc1]

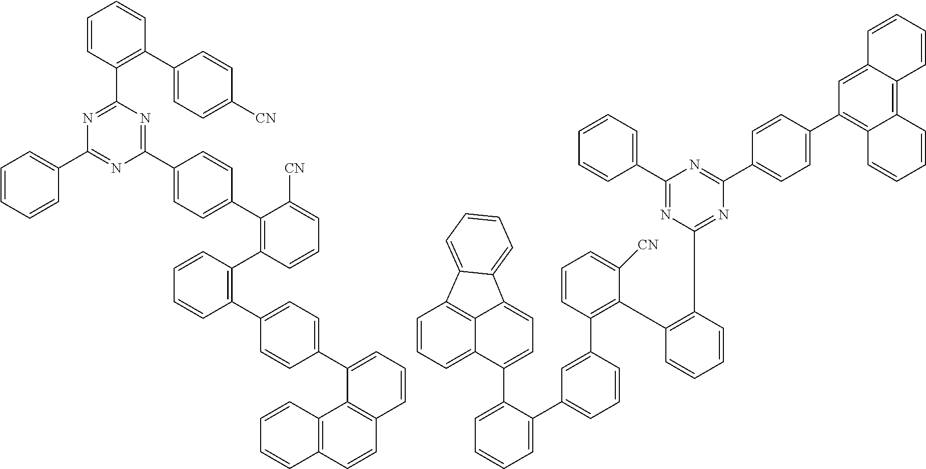

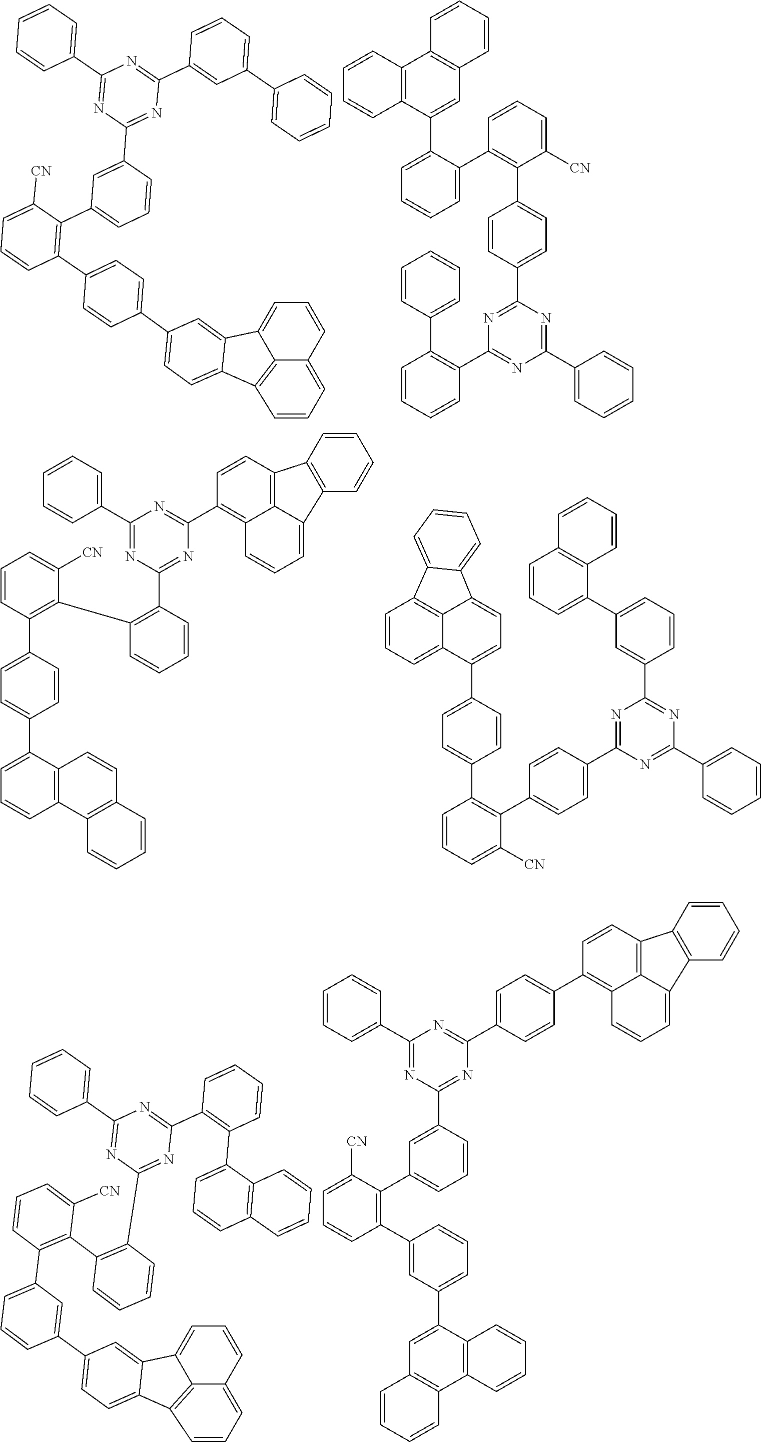

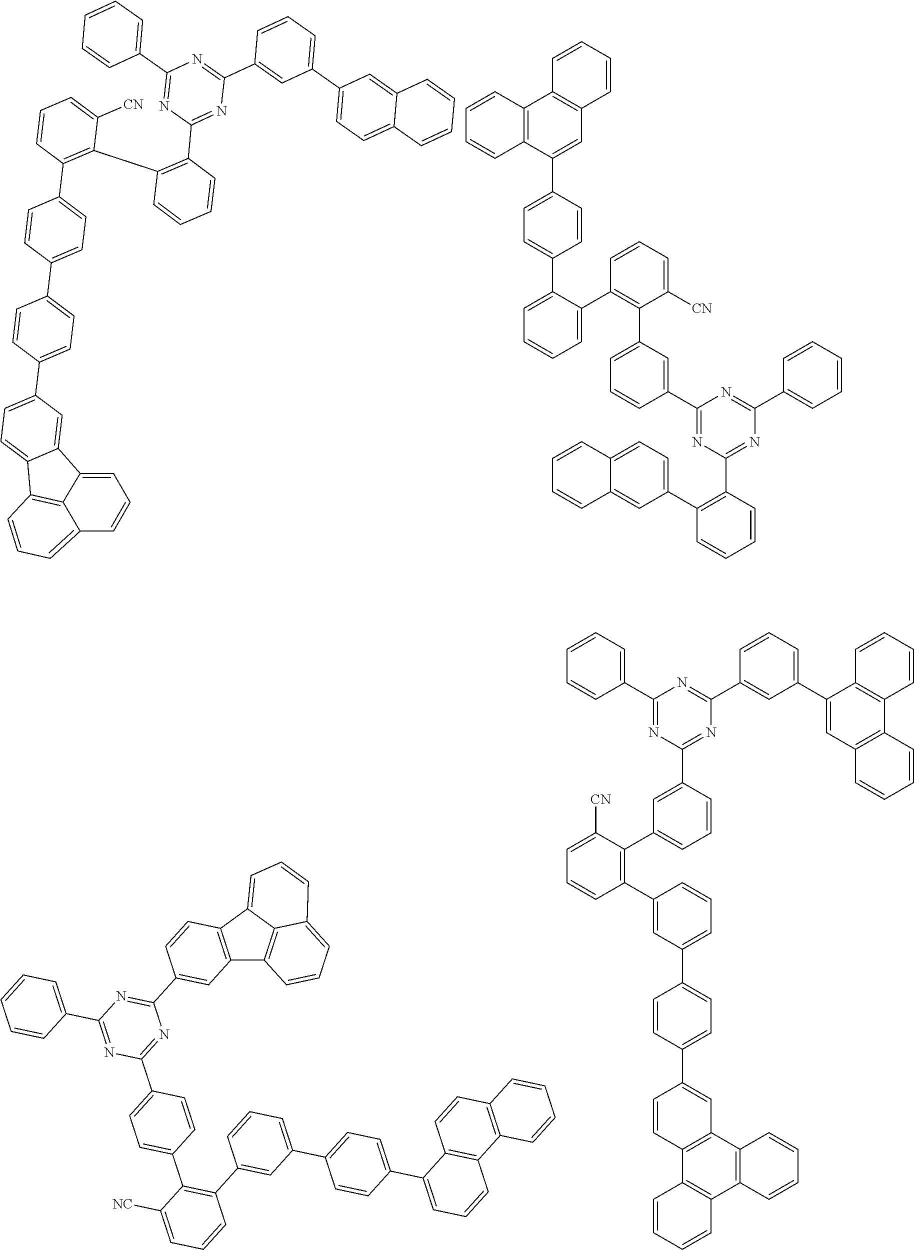

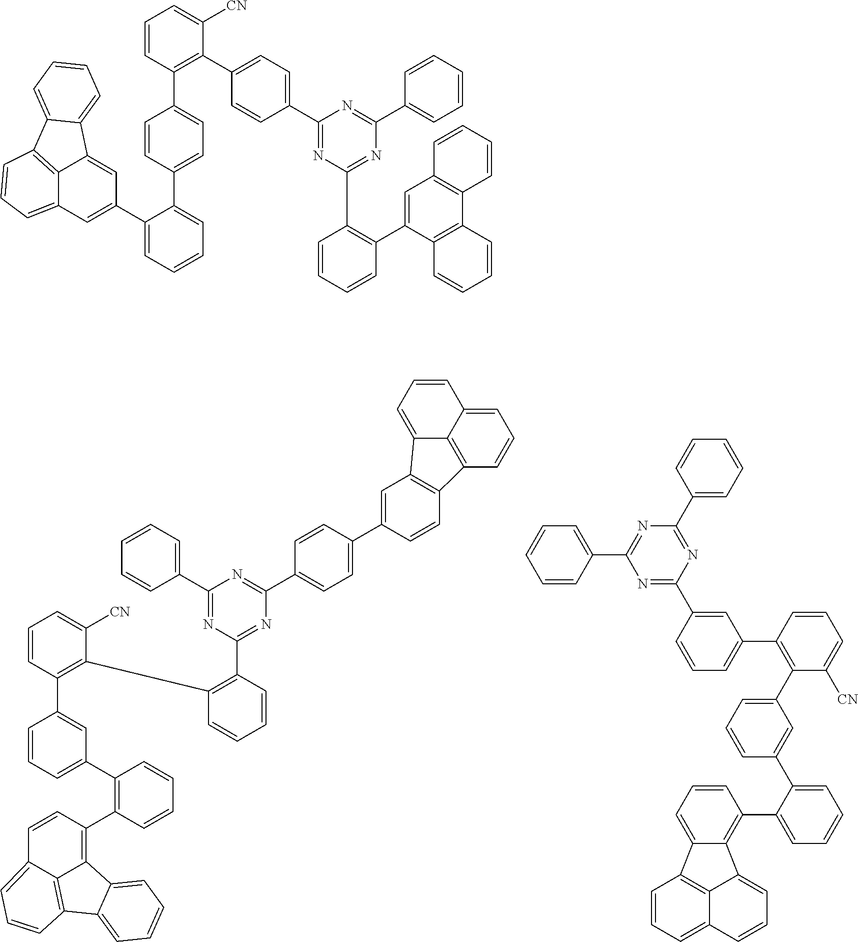

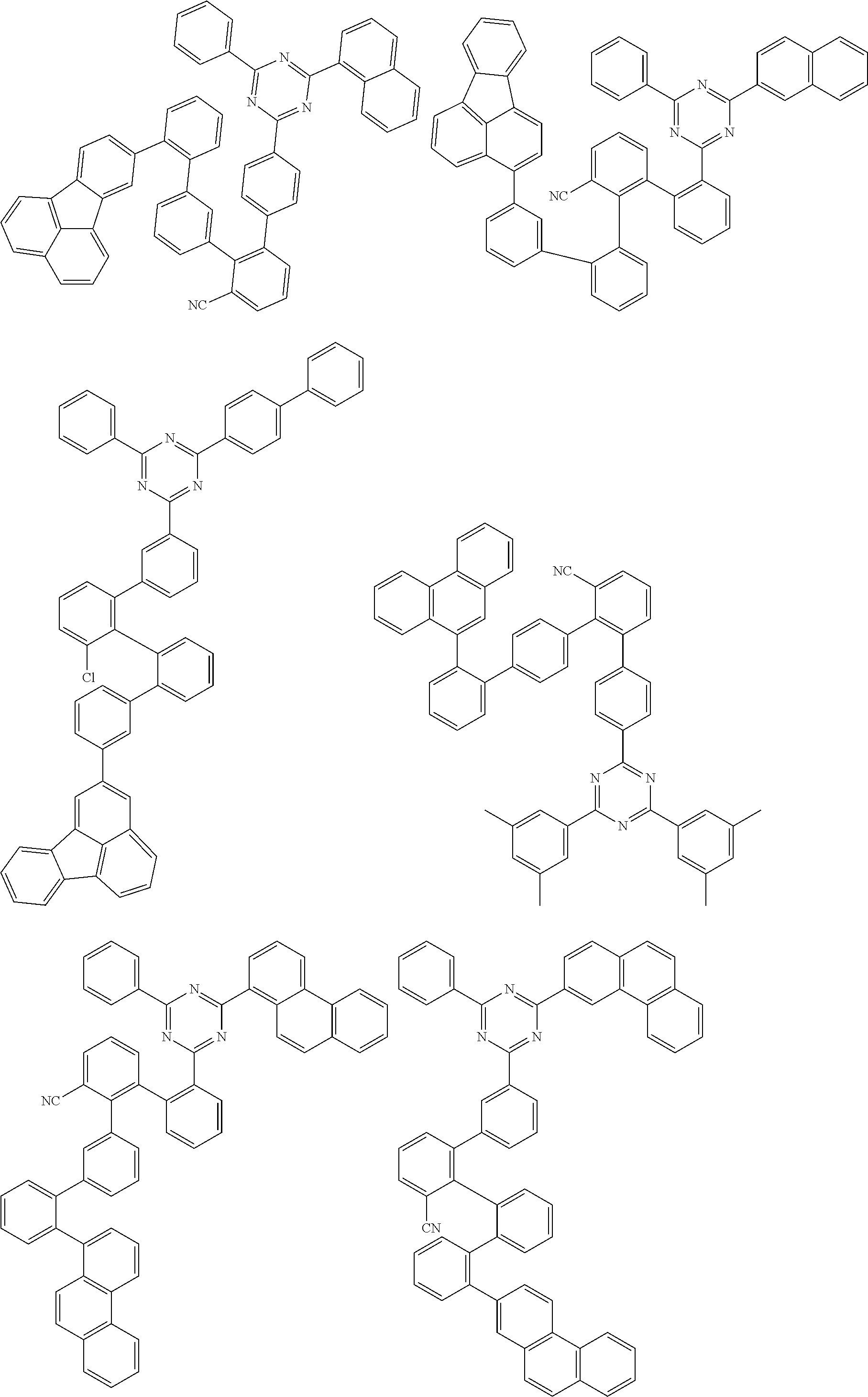

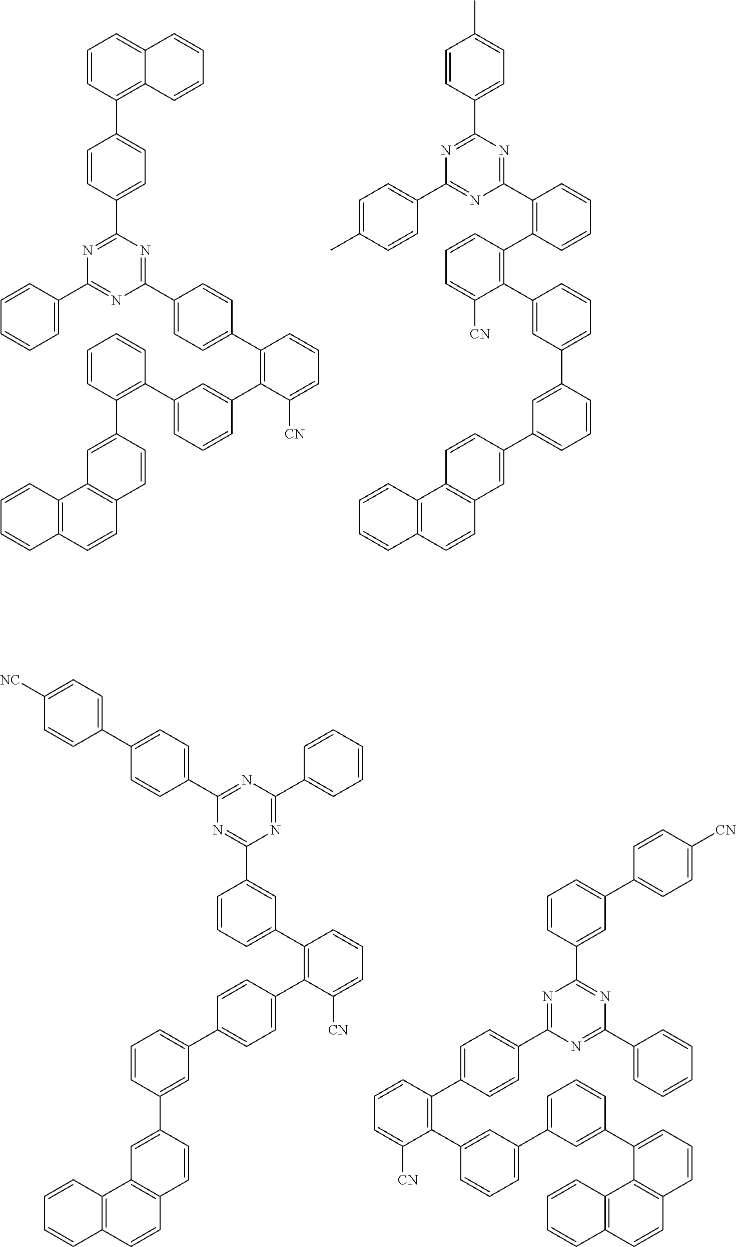

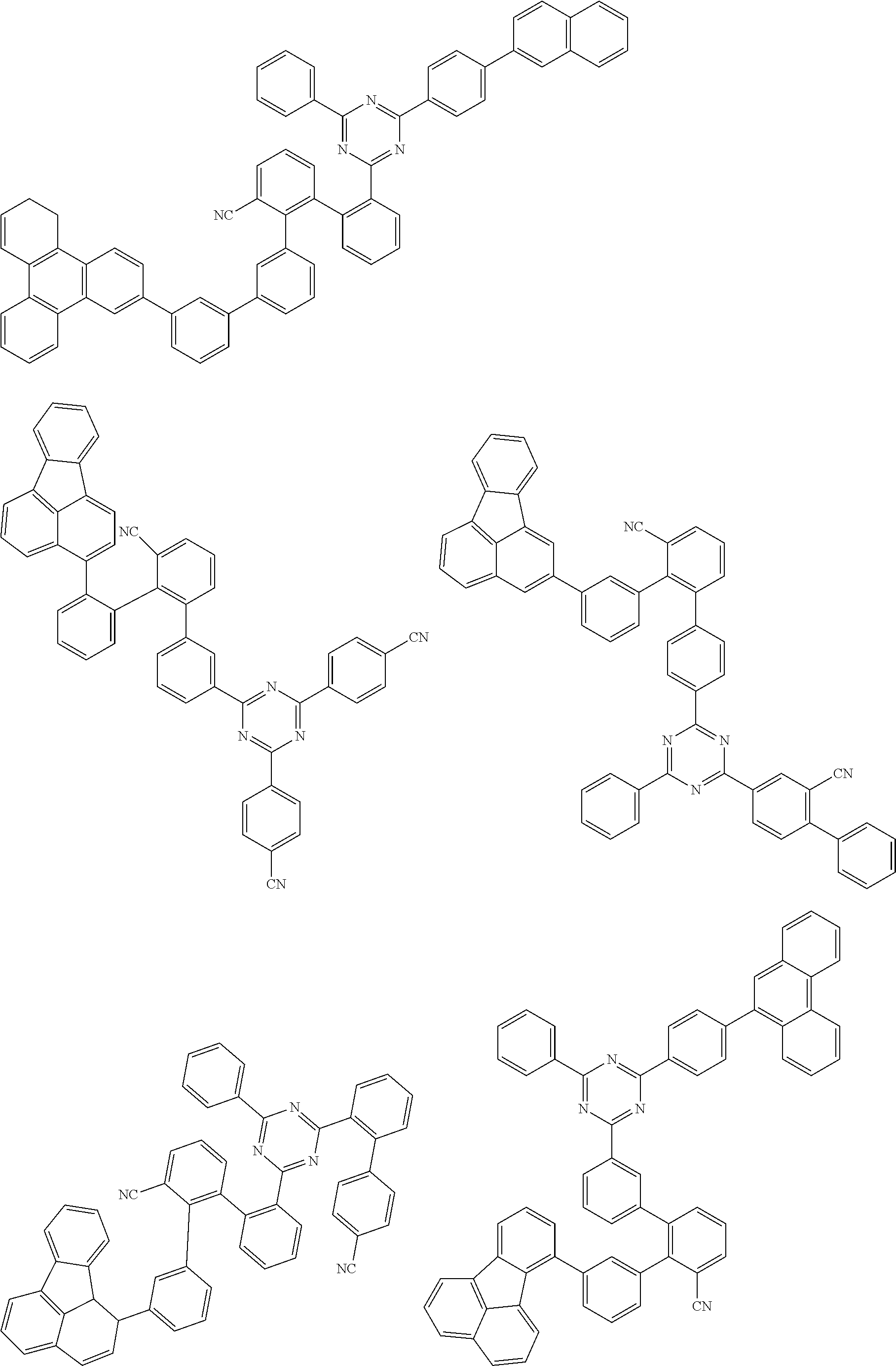

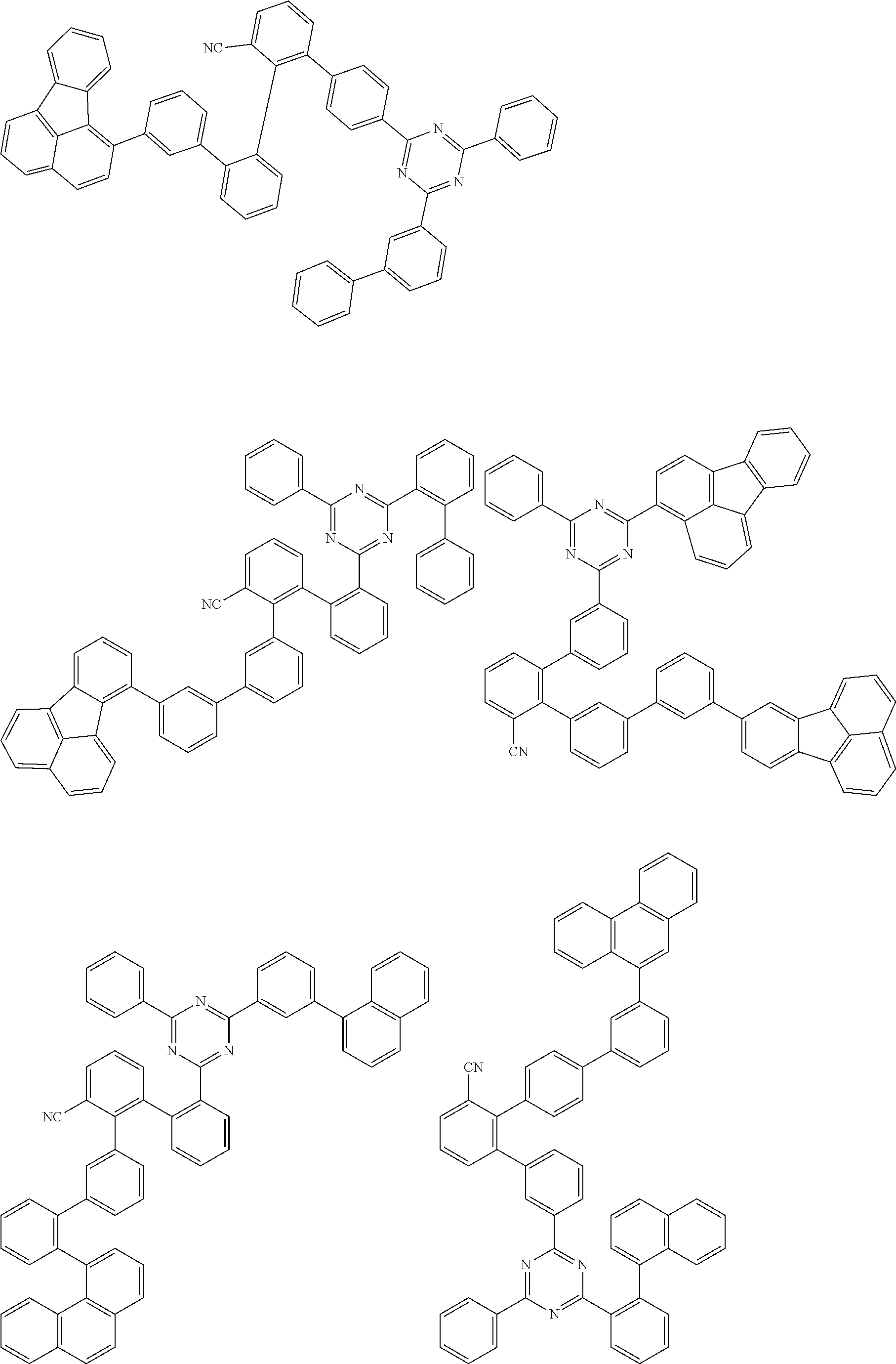

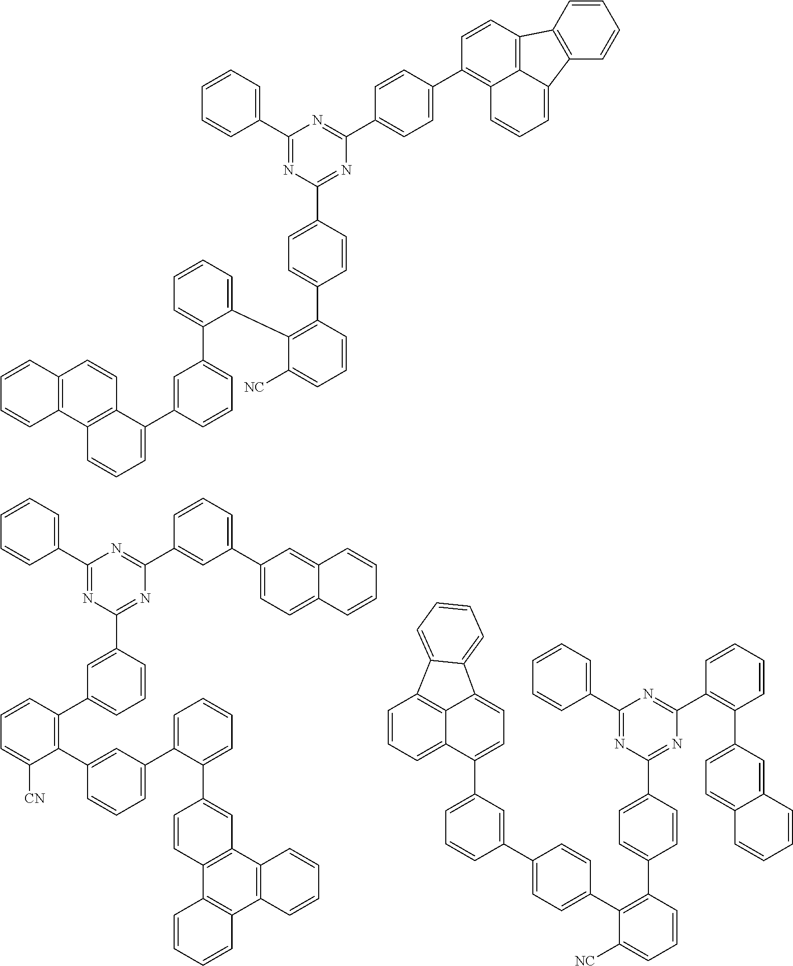 [SMILES: N#Cc1cccc(-c2ccc(-c3nc(-c4ccccc4)nc(-c4ccc(-c5ccc6c7c(cccc57)-c5ccccc5-6)cc4)n3)cc2)c1-c1ccccc1-c1cccc(-c2cccc3c2ccc2ccccc23)c1 ; N#Cc1cccc(-c2cccc(-c3nc(-c4ccccc4)nc(-c4cccc(-c5ccc6ccccc6c5)c4)n3)c2)c1-c1cccc(-c2ccccc2-c2ccc3c4ccccc4c4ccccc4c3c2)c1 ; N#Cc1cccc(-c2ccc(-c3nc(-c4ccccc4)nc(-c4ccccc4-c4ccc5ccccc5c4)n3)cc2)c1-c1ccc(-c2cccc(-c3ccc4c5c(cccc35)-c3ccccc3-4)c2)cc1]

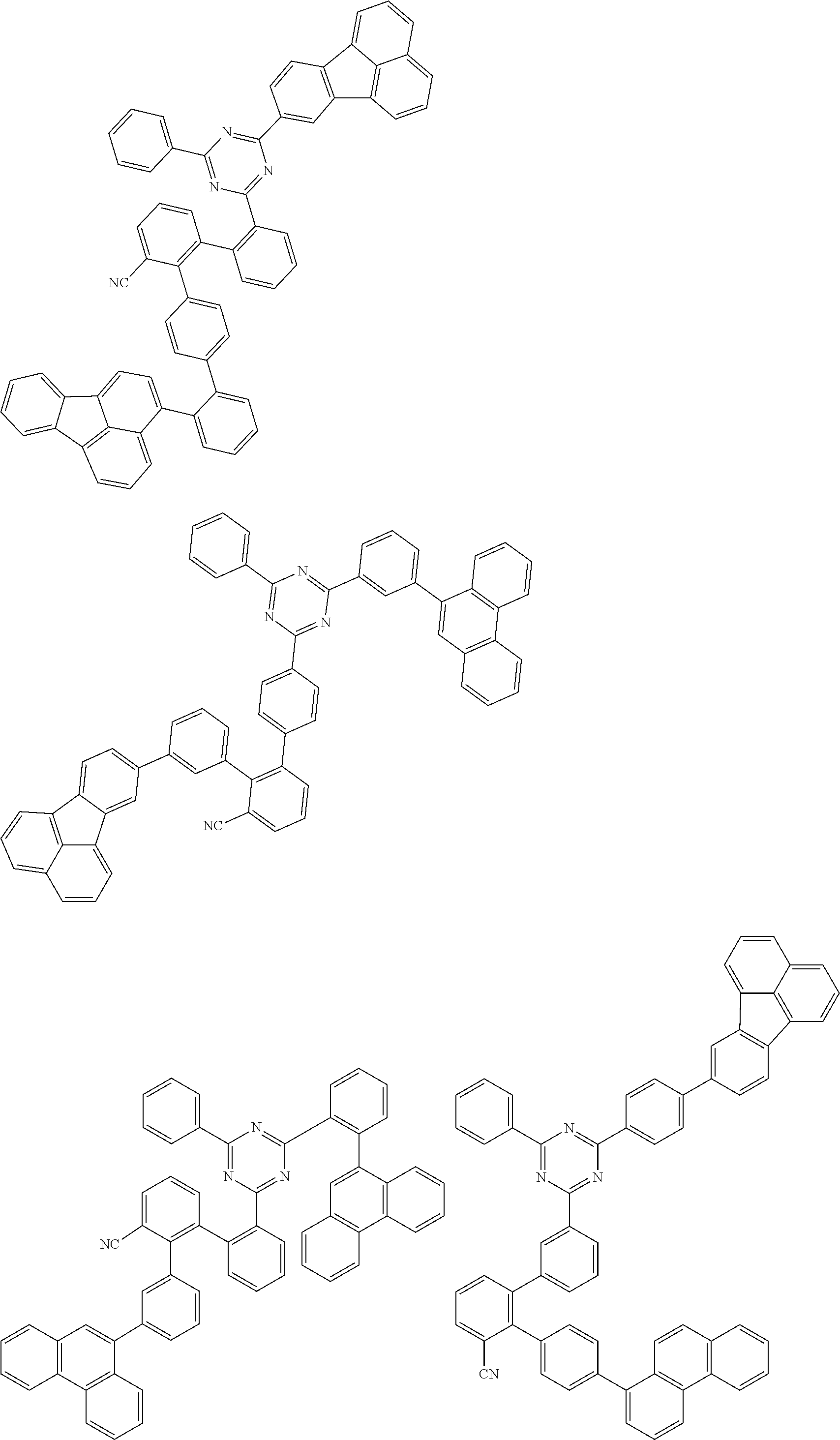 [SMILES: N#Cc1cccc(-c2ccccc2-c2nc(-c3ccccc3)nc(-c3ccc4c(c3)-c3cccc5cccc-4c35)n2)c1-c1ccc(-c2ccccc2-c2ccc3c4c(cccc24)-c2ccccc2-3)cc1 ; N#Cc1cccc(-c2ccc(-c3nc(-c4ccccc4)nc(-c4cccc(-c5cc6ccccc6c6ccccc56)c4)n3)cc2)c1-c1cccc(-c2ccc3c(c2)-c2cccc4cccc-3c24)c1 ; N#Cc1cccc(-c2ccccc2-c2nc(-c3ccccc3)nc(-c3ccccc3-c3cc4ccccc4c4ccccc34)n2)c1-c1cccc(-c2cc3ccccc3c3ccccc23)c1 ; N#Cc1cccc(-c2cccc(-c3nc(-c4ccccc4)nc(-c4ccc(-c5ccc6c(c5)-c5cccc7cccc-6c57)cc4)n3)c2)c1-c1ccc(-c2cccc3c2ccc2ccccc23)cc1]

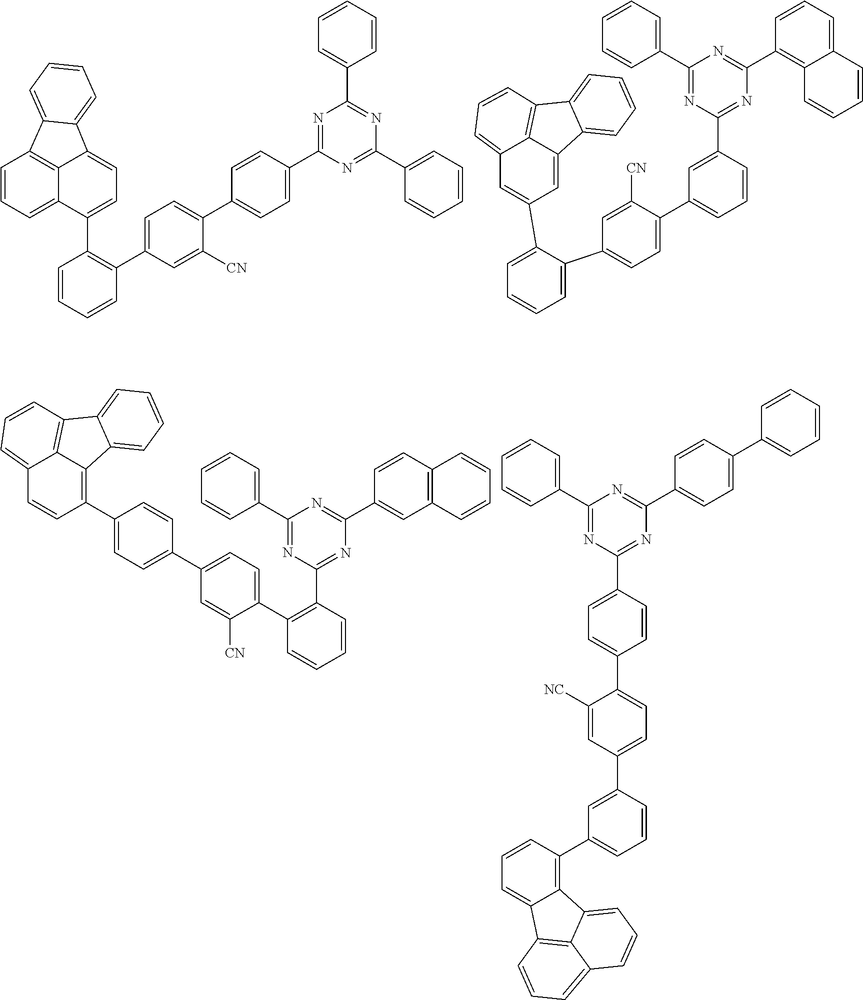

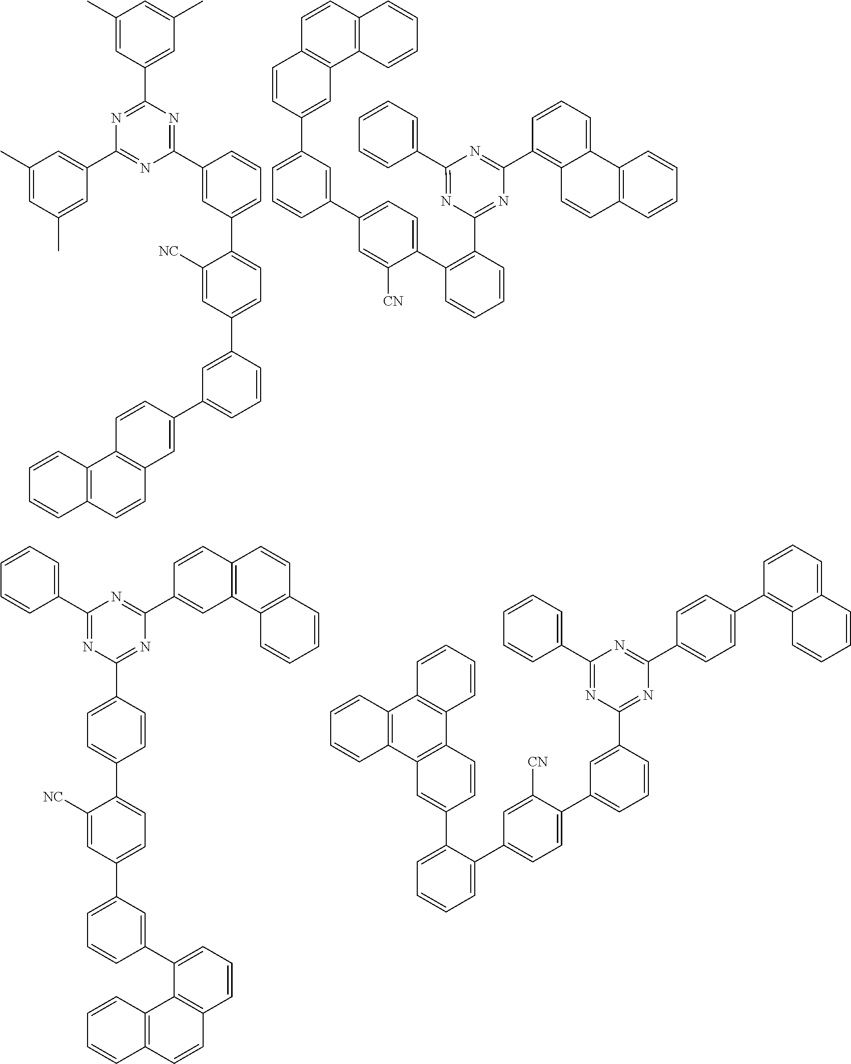

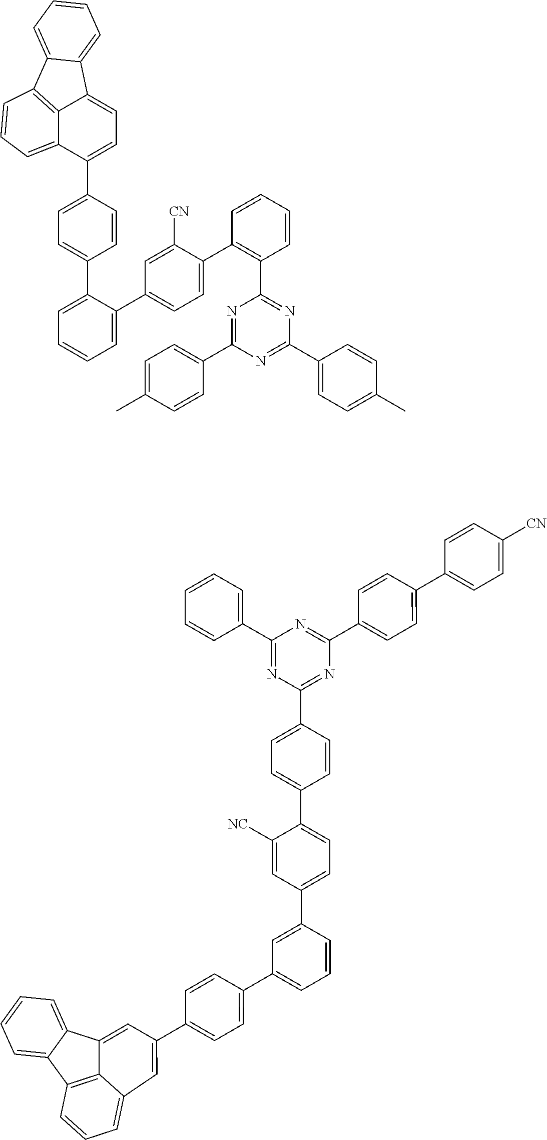 [SMILES: Cc1ccc(-c2nc(-c3ccc(C)cc3)nc(-c3ccccc3-c3ccc(-c4ccccc4-c4ccc(-c5ccc6c7c(cccc57)-c5ccccc5-6)cc4)cc3C#N)n2)cc1 ; N#Cc1ccc(-c2ccc(-c3nc(-c4ccccc4)nc(-c4ccc(-c5ccc(-c6cccc(-c7ccc(-c8cc9c%10c(cccc%10c8)-c8ccccc8-9)cc7)c6)cc5C#N)cc4)n3)cc2)cc1]

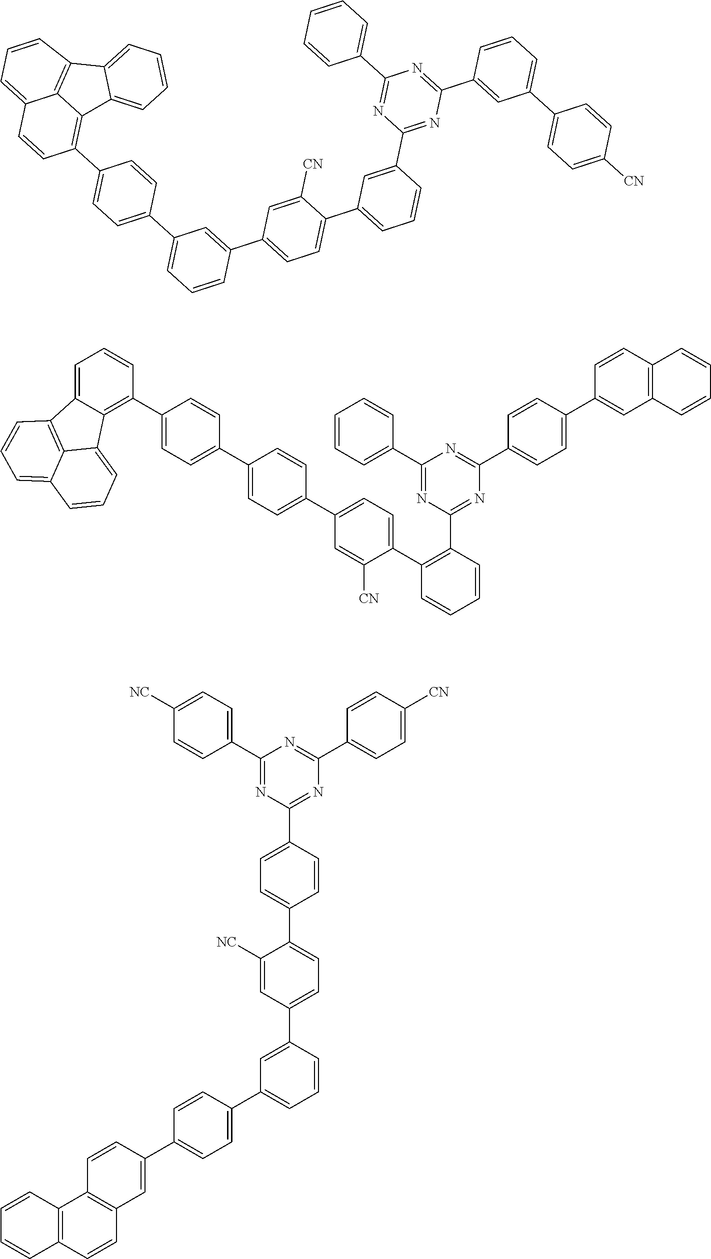

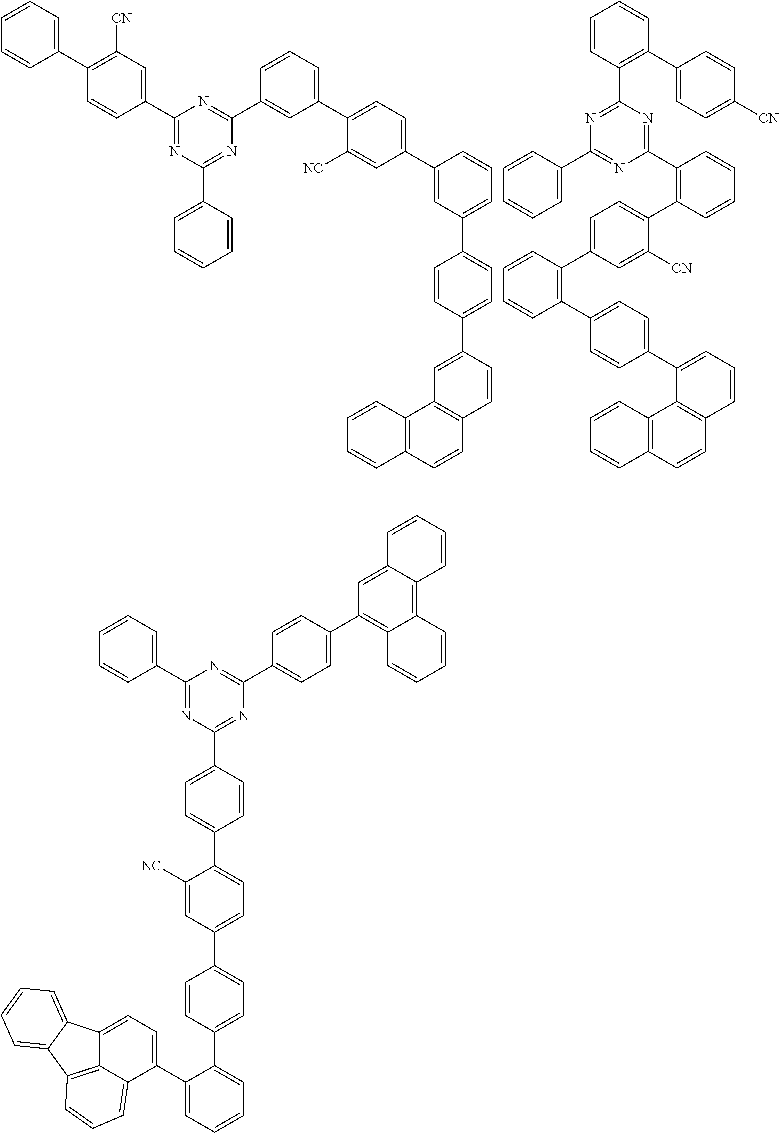

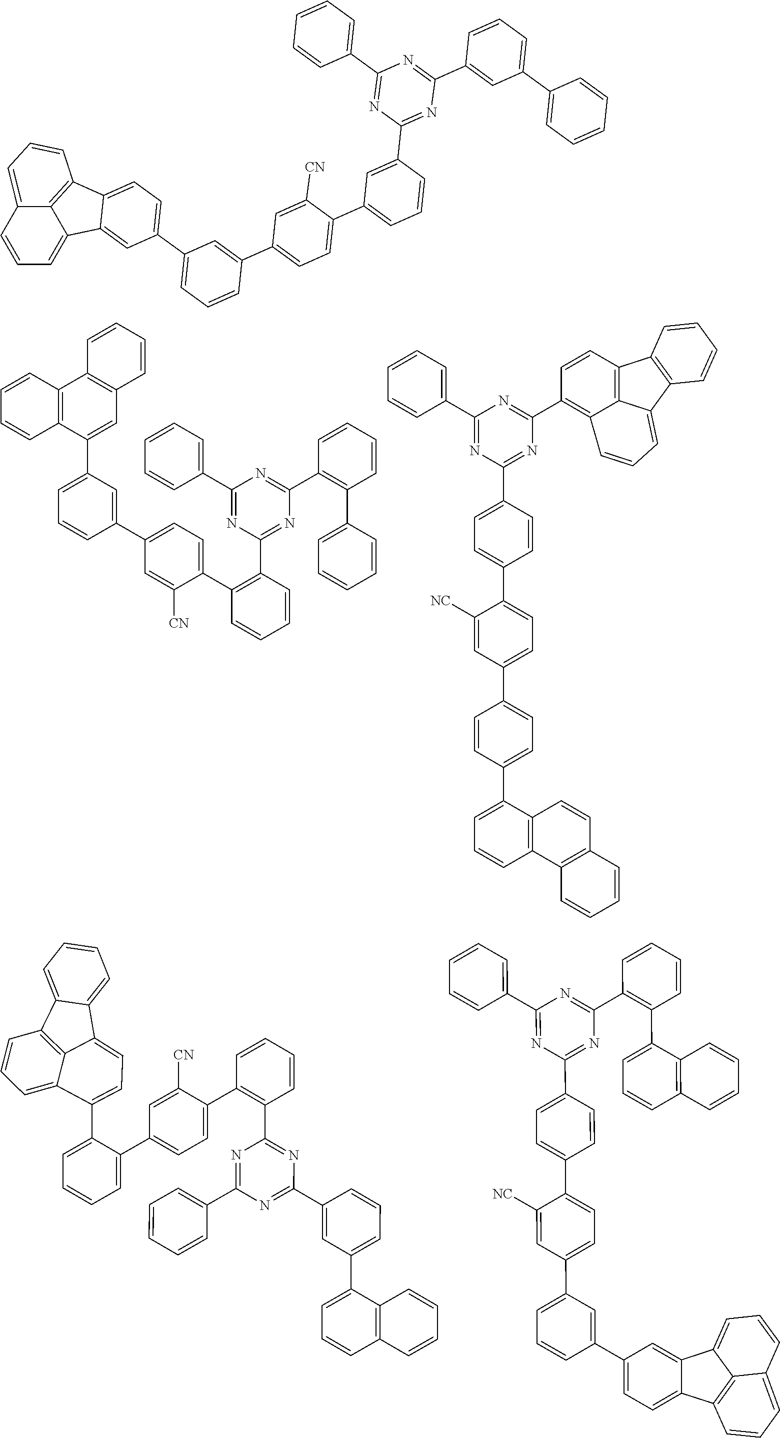

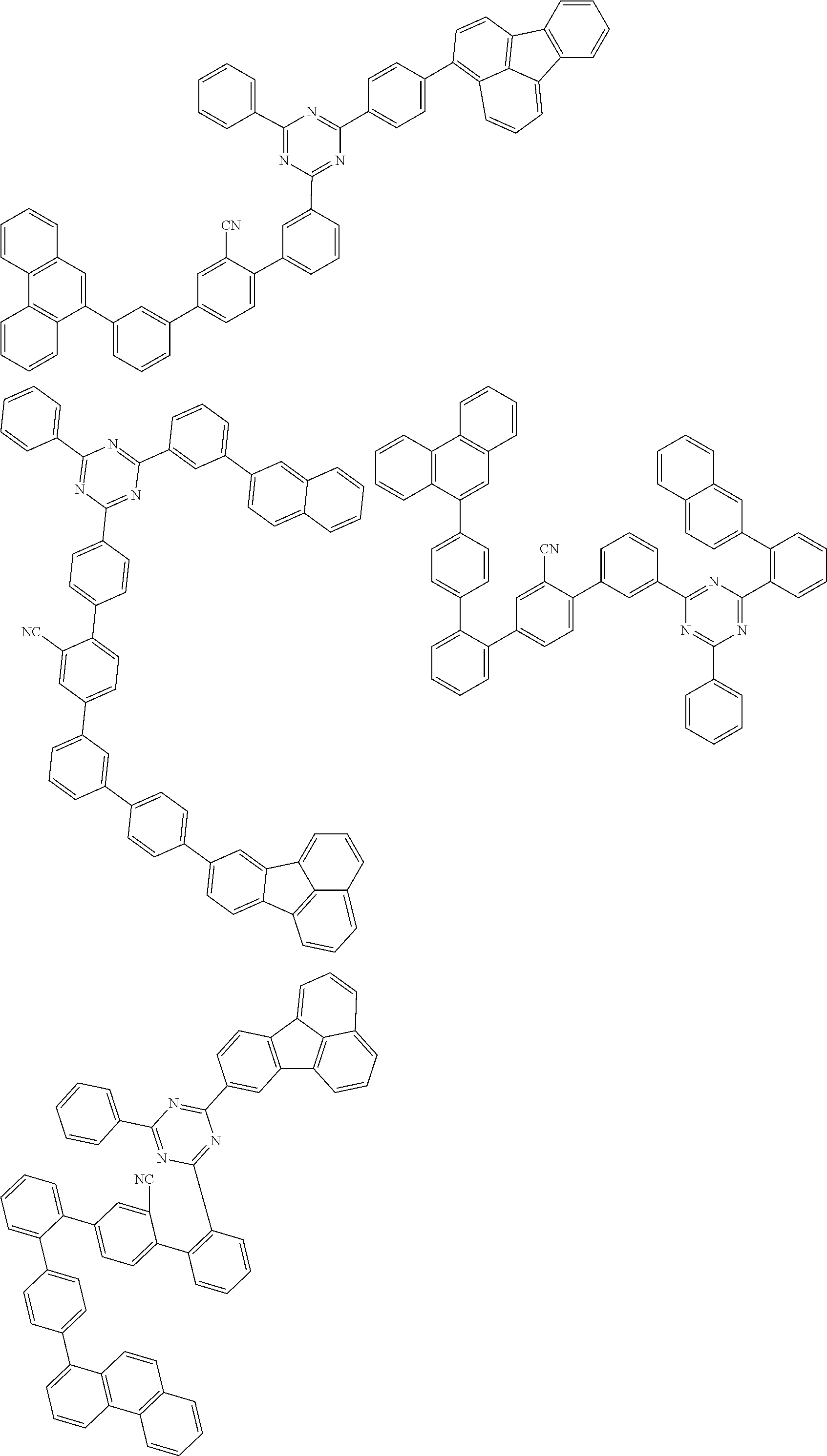

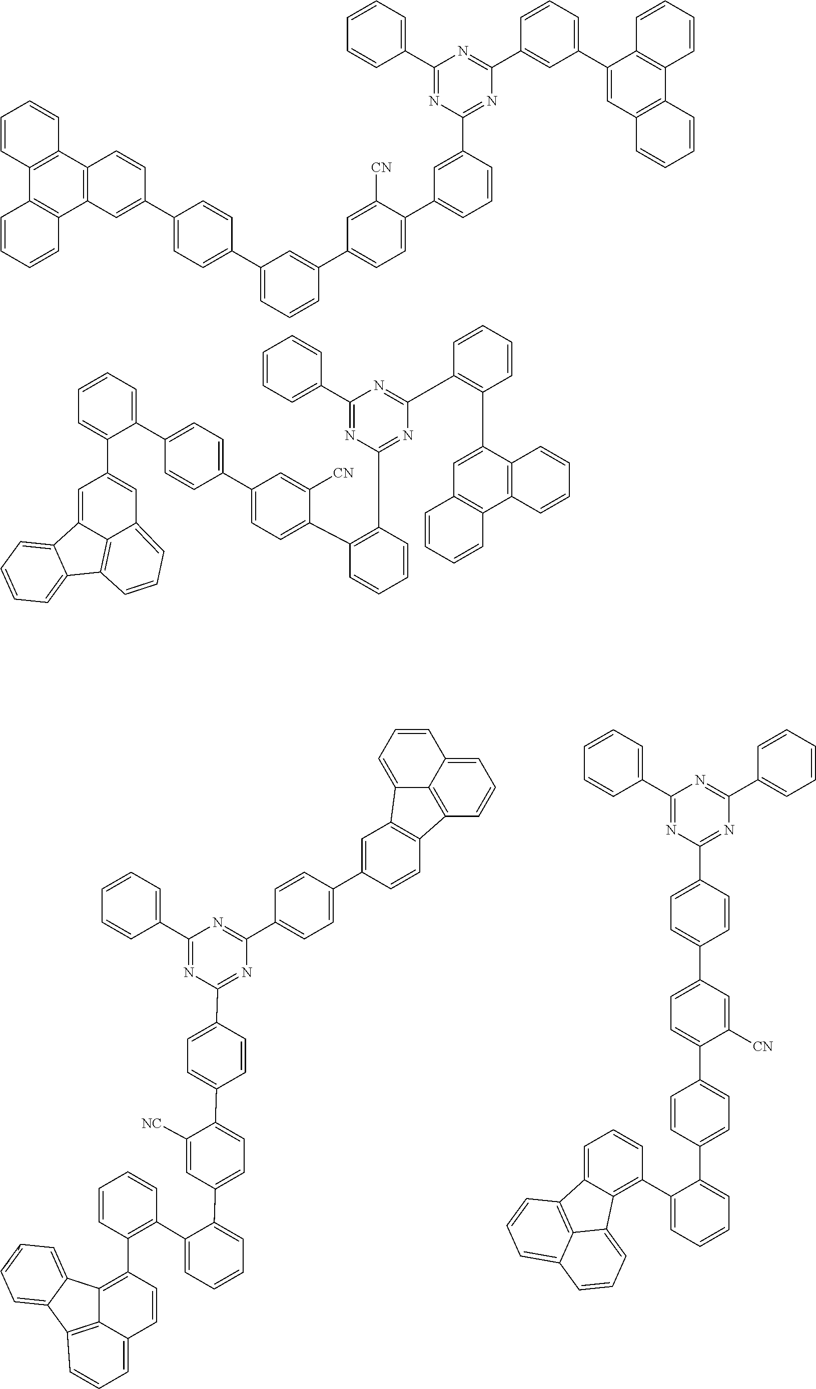

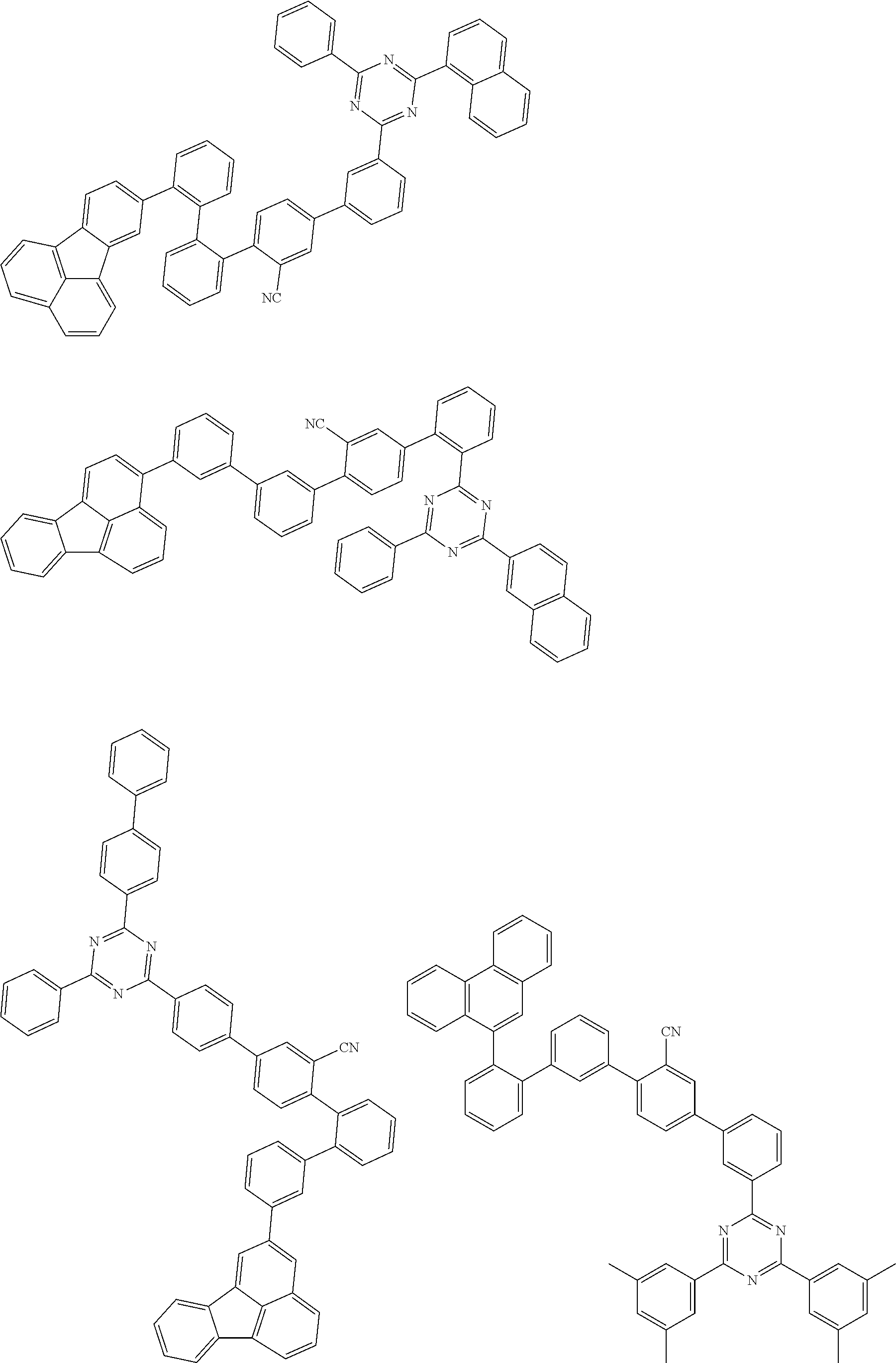 [SMILES: N#Cc1cc(-c2cccc(-c3nc(-c4ccccc4)nc(-c4cccc5ccccc45)n3)c2)ccc1-c1ccccc1-c1ccccc1-c1ccc2c(c1)-c1cccc3cccc-2c13 ; N#Cc1cc(-c2ccccc2-c2nc(-c3ccccc3)nc(-c3ccc4ccccc4c3)n2)ccc1-c1cccc(-c2cccc(-c3ccc4c5c(cccc35)-c3ccccc3-4)c2)c1 ; N#Cc1cc(-c2ccc(-c3nc(-c4ccccc4)nc(-c4ccc(-c5ccccc5)cc4)n3)cc2)ccc1-c1ccccc1-c1cccc(-c2cc3c4c(cccc4c2)-c2ccccc2-3)c1 ; Cc1cc(C)cc(-c2nc(-c3cc(C)cc(C)c3)nc(-c3cccc(-c4ccc(-c5cccc(-c6ccccc6-c6cc7ccccc7c7ccccc67)c5)c(C#N)c4)c3)n2)c1]

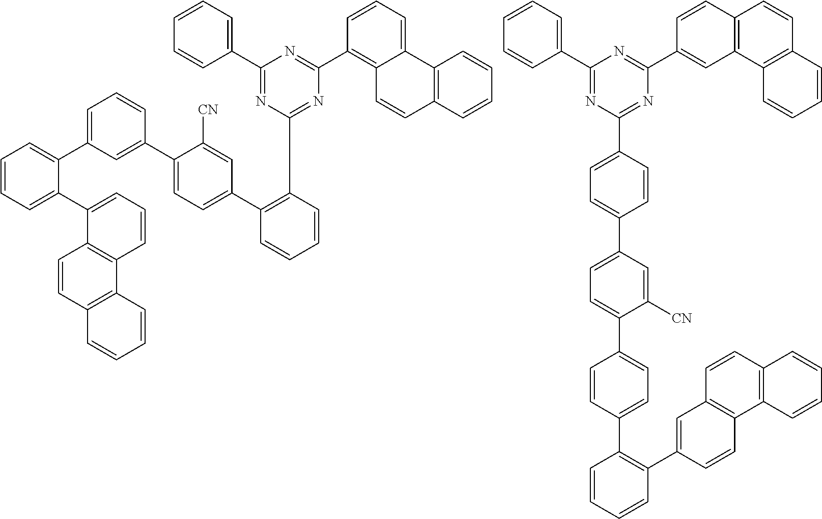

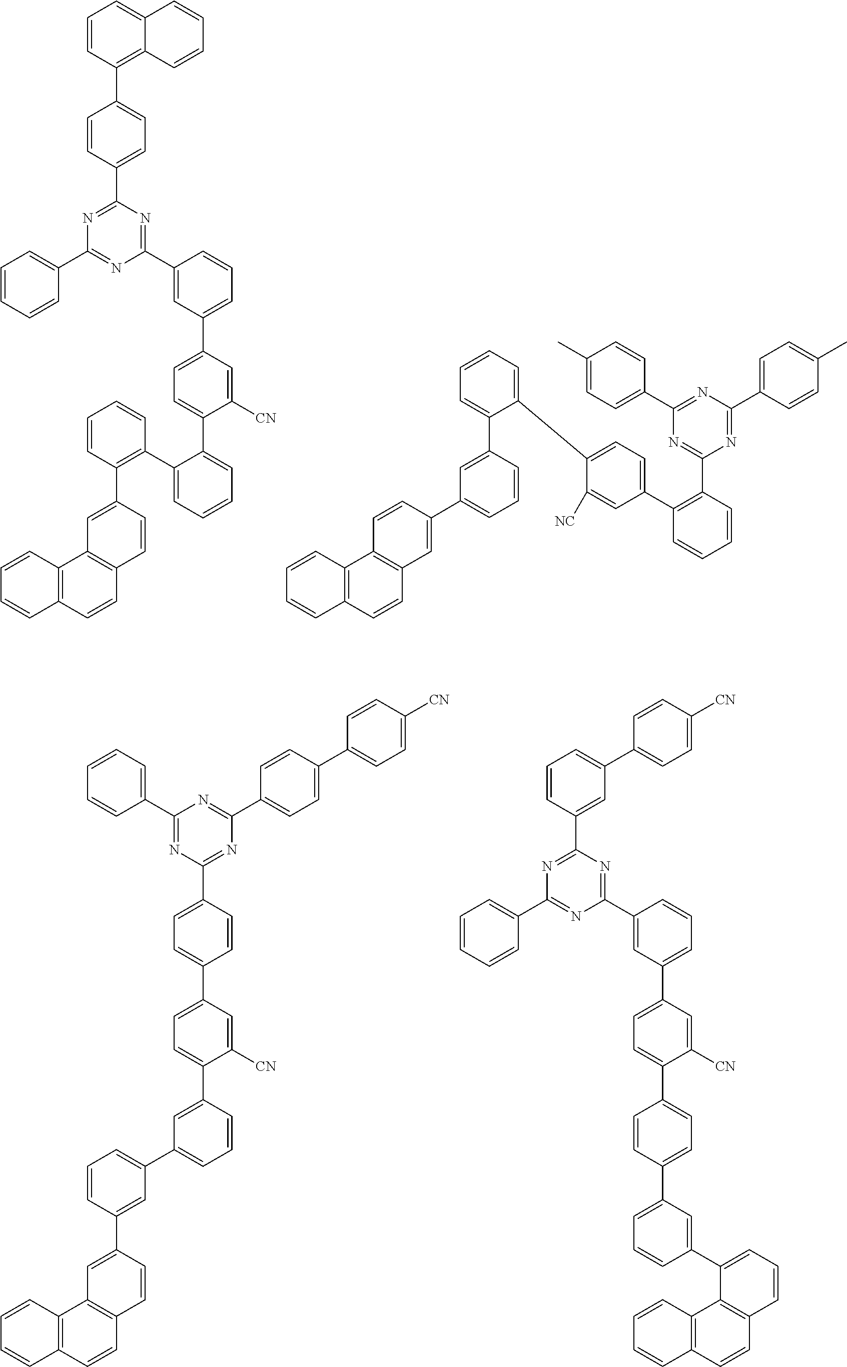 [SMILES: N#Cc1cc(-c2cccc(-c3nc(-c4ccccc4)nc(-c4ccc(-c5cccc6ccccc56)cc4)n3)c2)ccc1-c1ccccc1-c1ccccc1-c1ccc2ccc3ccccc3c2c1 ; Cc1ccc(-c2nc(-c3ccc(C)cc3)nc(-c3ccccc3-c3ccc(-c4ccccc4-c4cccc(-c5ccc6c(ccc7ccccc76)c5)c4)c(C#N)c3)n2)cc1 ; N#Cc1ccc(-c2ccc(-c3nc(-c4ccccc4)nc(-c4ccc(-c5ccc(-c6cccc(-c7cccc(-c8ccc9ccc%10ccccc%10c9c8)c7)c6)c(C#N)c5)cc4)n3)cc2)cc1 ; N#Cc1ccc(-c2cccc(-c3nc(-c4ccccc4)nc(-c4cccc(-c5ccc(-c6ccc(-c7cccc(-c8cccc9ccc%10ccccc%10c89)c7)cc6)c(C#N)c5)c4)n3)c2)cc1]

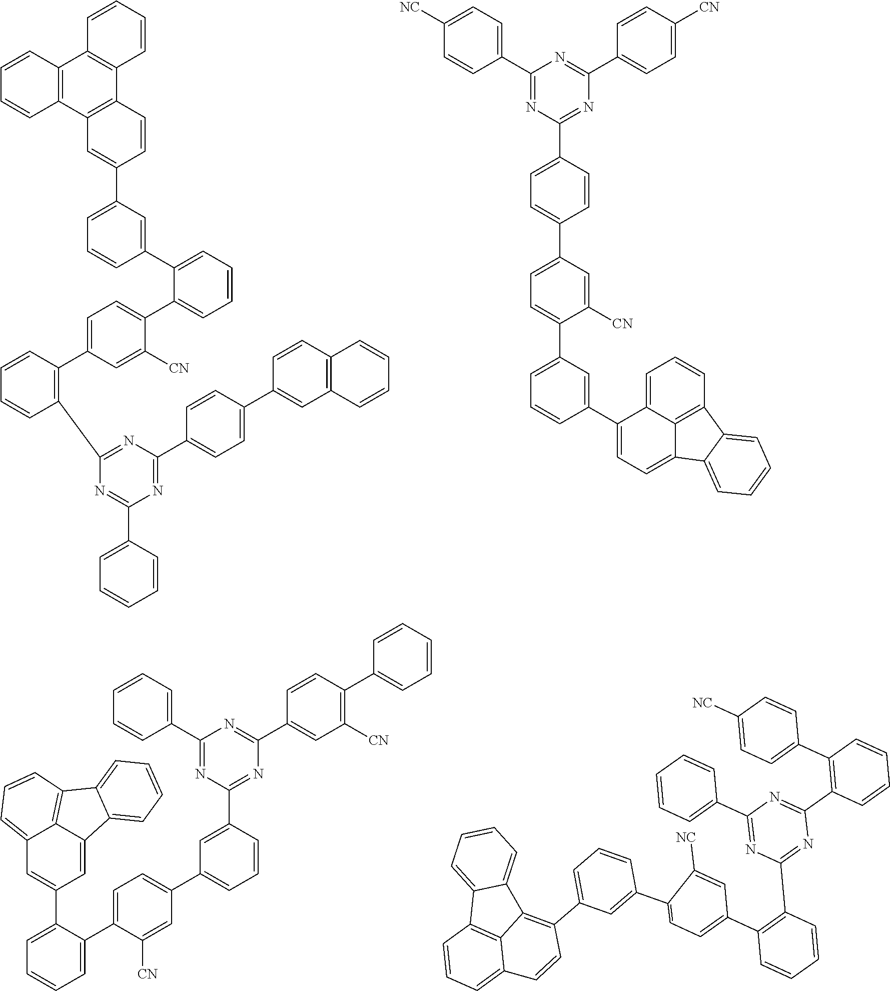 [SMILES: N#Cc1cc(-c2ccccc2-c2nc(-c3ccccc3)nc(-c3ccc(-c4ccc5ccccc5c4)cc3)n2)ccc1-c1ccccc1-c1cccc(-c2ccc3c4ccccc4c4ccccc4c3c2)c1 ; N#Cc1ccc(-c2nc(-c3ccc(C#N)cc3)nc(-c3ccc(-c4ccc(-c5cccc(-c6ccc7c8c(cccc68)-c6ccccc6-7)c5)c(C#N)c4)cc3)n2)cc1 ; N#Cc1cc(-c2nc(-c3ccccc3)nc(-c3cccc(-c4ccc(-c5ccccc5-c5cc6c7c(cccc7c5)-c5ccccc5-6)c(C#N)c4)c3)n2)ccc1-c1ccccc1 ; N#Cc1ccc(-c2ccccc2-c2nc(-c3ccccc3)nc(-c3ccccc3-c3ccc(-c4cccc(-c5ccc6cccc7c6c5-c5ccccc5-7)c4)c(C#N)c3)n2)cc1]

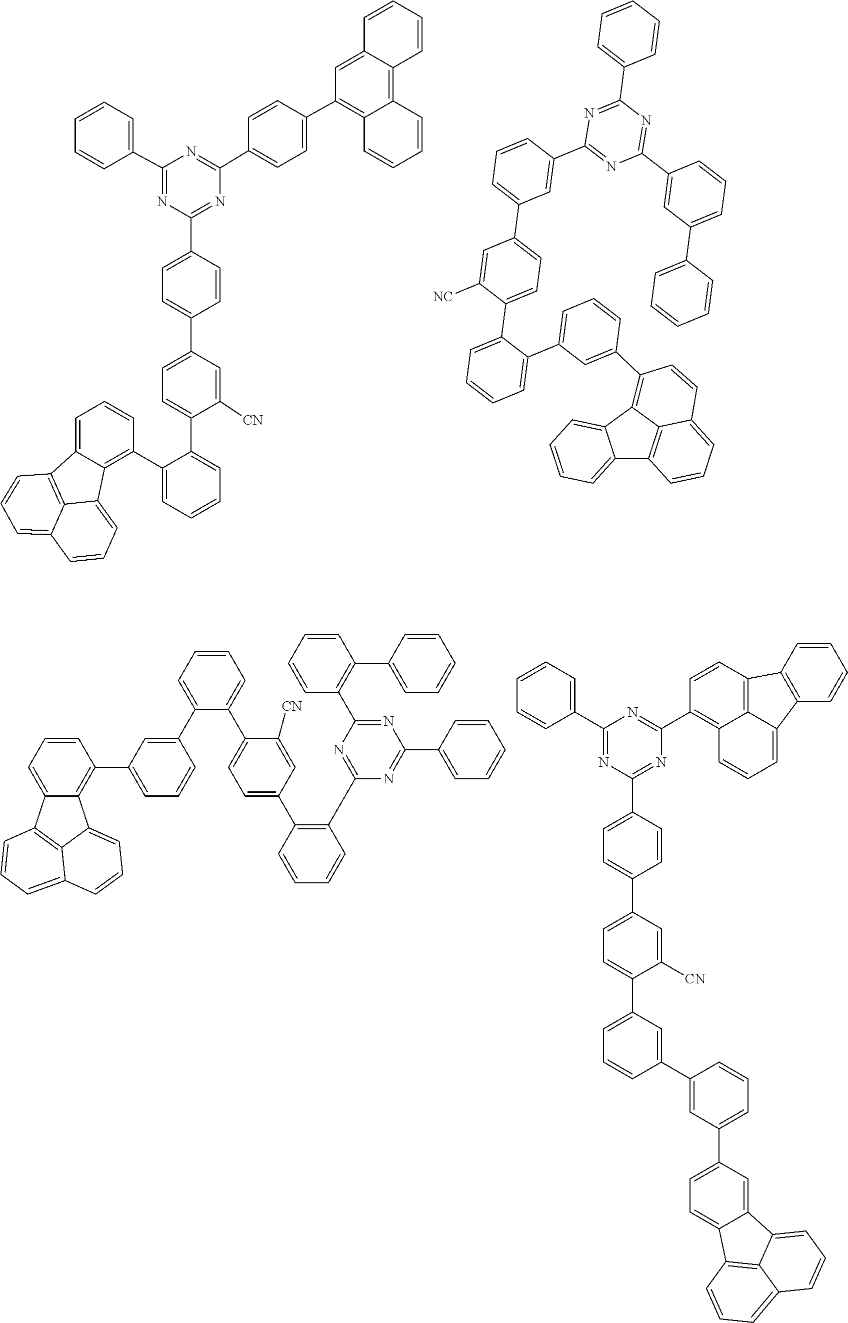

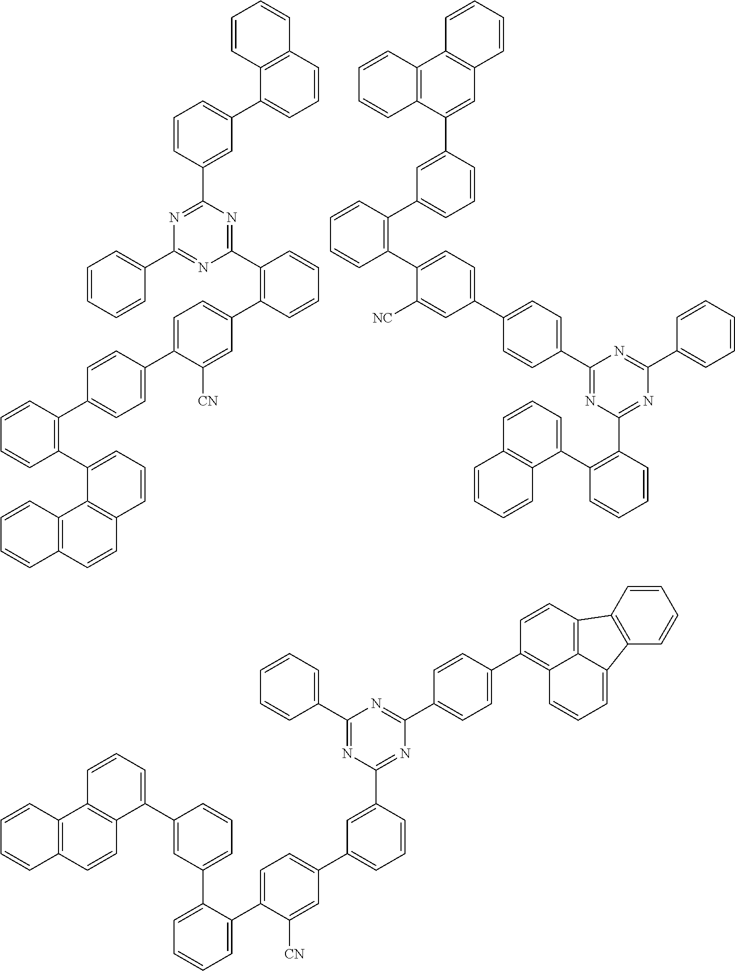

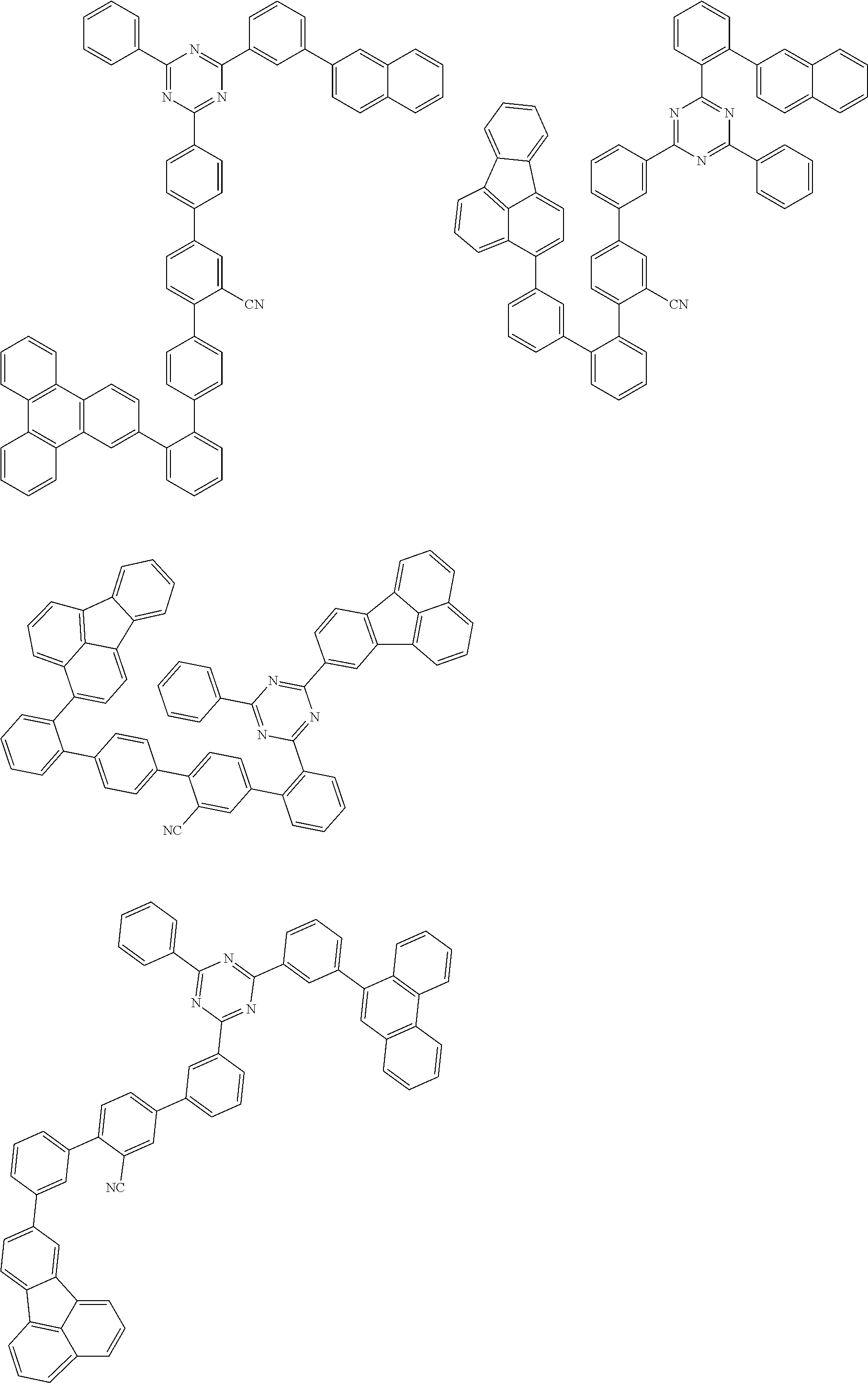 [SMILES: N#Cc1cc(-c2ccc(-c3nc(-c4ccccc4)nc(-c4cccc(-c5ccc6ccccc6c5)c4)n3)cc2)ccc1-c1ccc(-c2ccccc2-c2ccc3c4ccccc4c4ccccc4c3c2)cc1 ; N#Cc1cc(-c2cccc(-c3nc(-c4ccccc4)nc(-c4ccccc4-c4ccc5ccccc5c4)n3)c2)ccc1-c1ccccc1-c1cccc(-c2ccc3c4c(cccc24)-c2ccccc2-3)c1 ; N#Cc1cc(-c2ccccc2-c2nc(-c3ccccc3)nc(-c3ccc4c(c3)-c3cccc5cccc-4c35)n2)ccc1-c1ccc(-c2ccccc2-c2ccc3c4c(cccc24)-c2ccccc2-3)cc1 ; N#Cc1cc(-c2cccc(-c3nc(-c4ccccc4)nc(-c4cccc(-c5cc6ccccc6c6ccccc56)c4)n3)c2)ccc1-c1cccc(-c2ccc3c(c2)-c2cccc4cccc-3c24)c1]

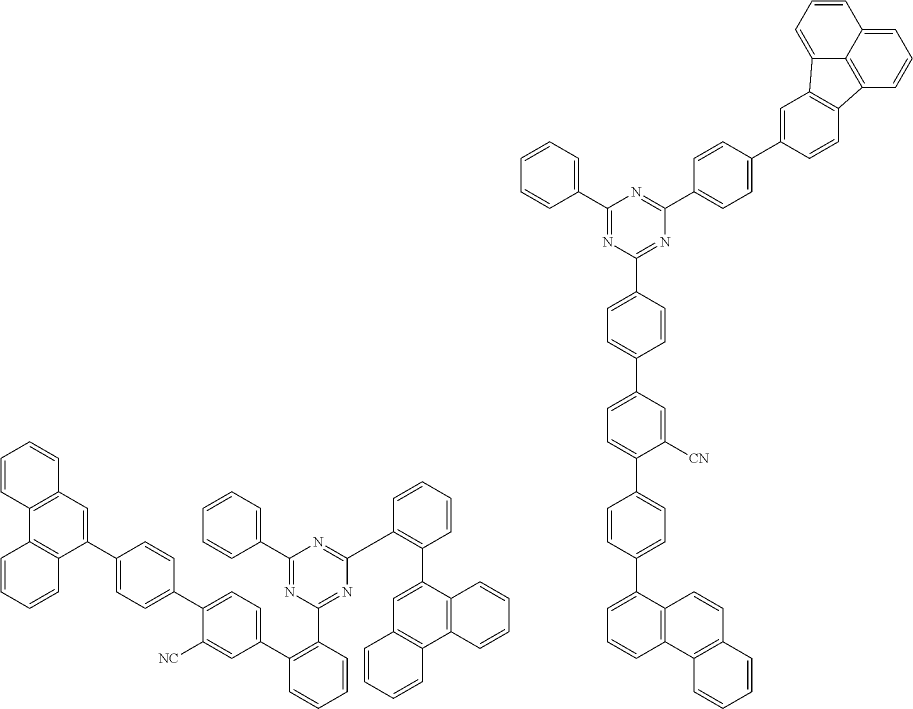

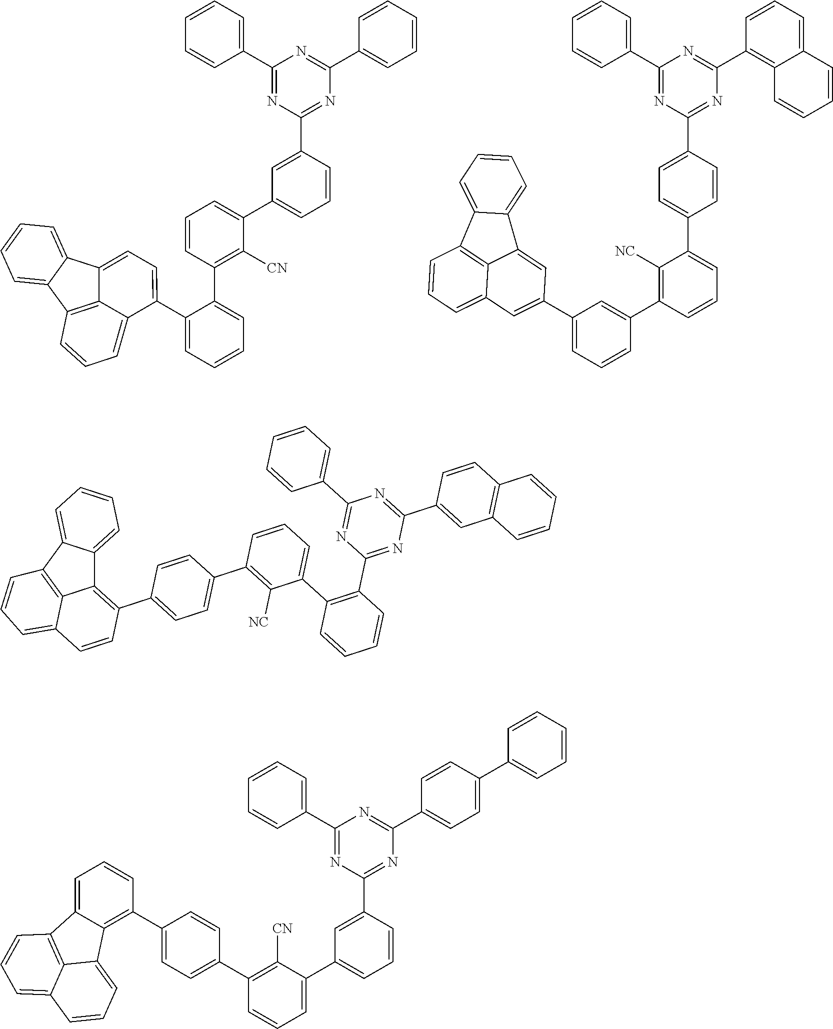

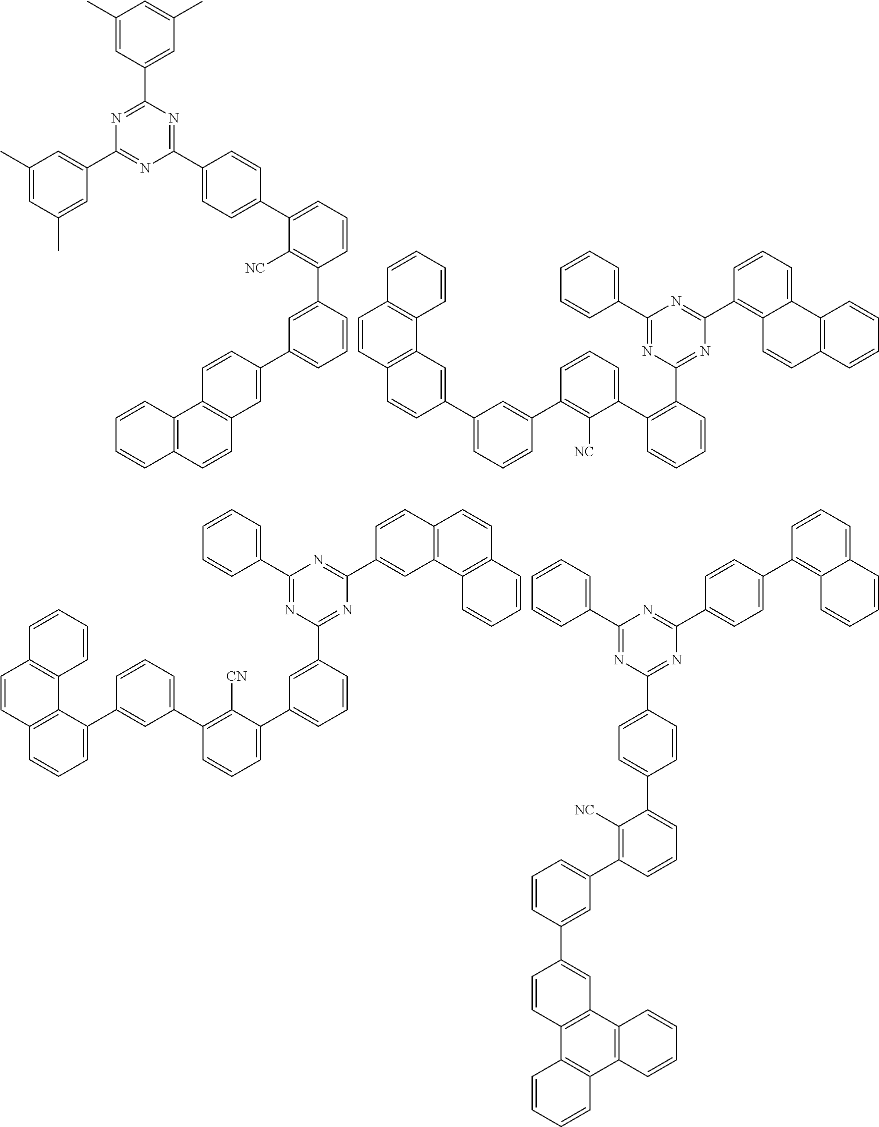

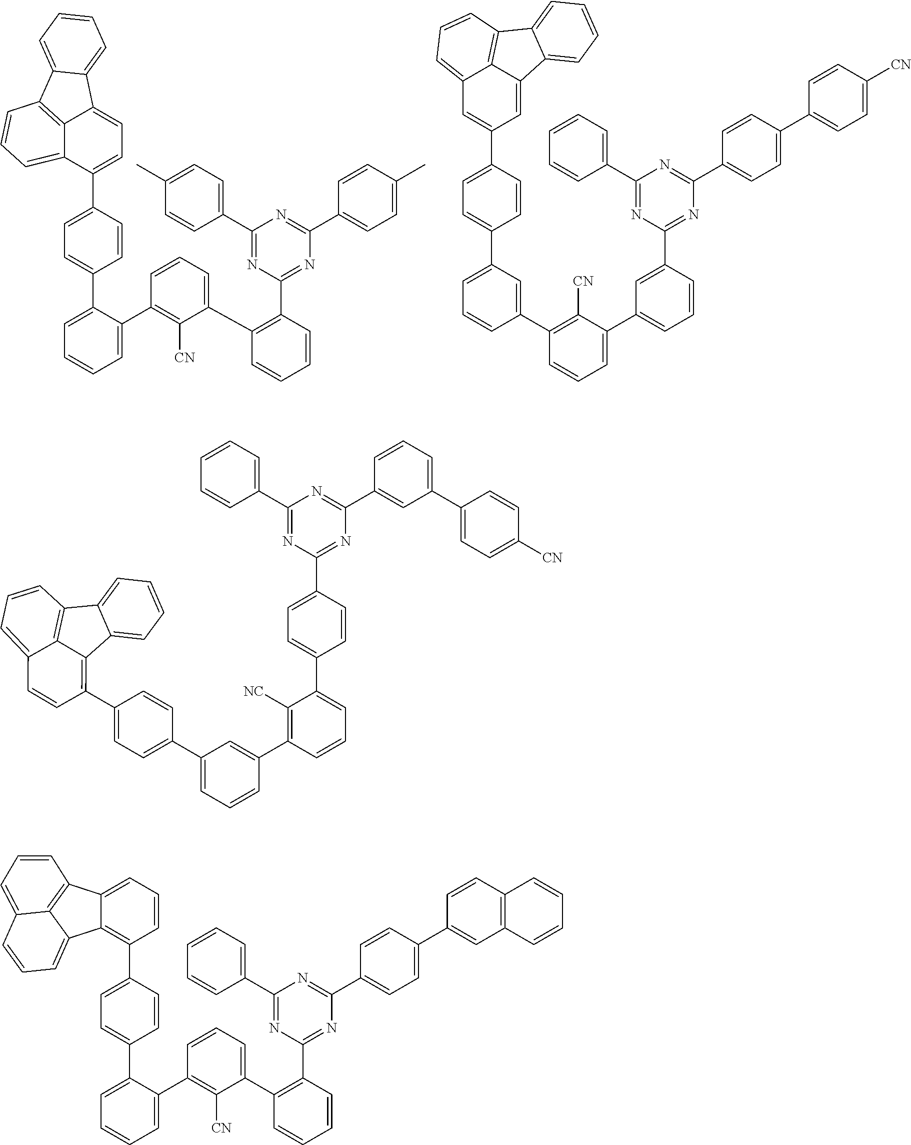

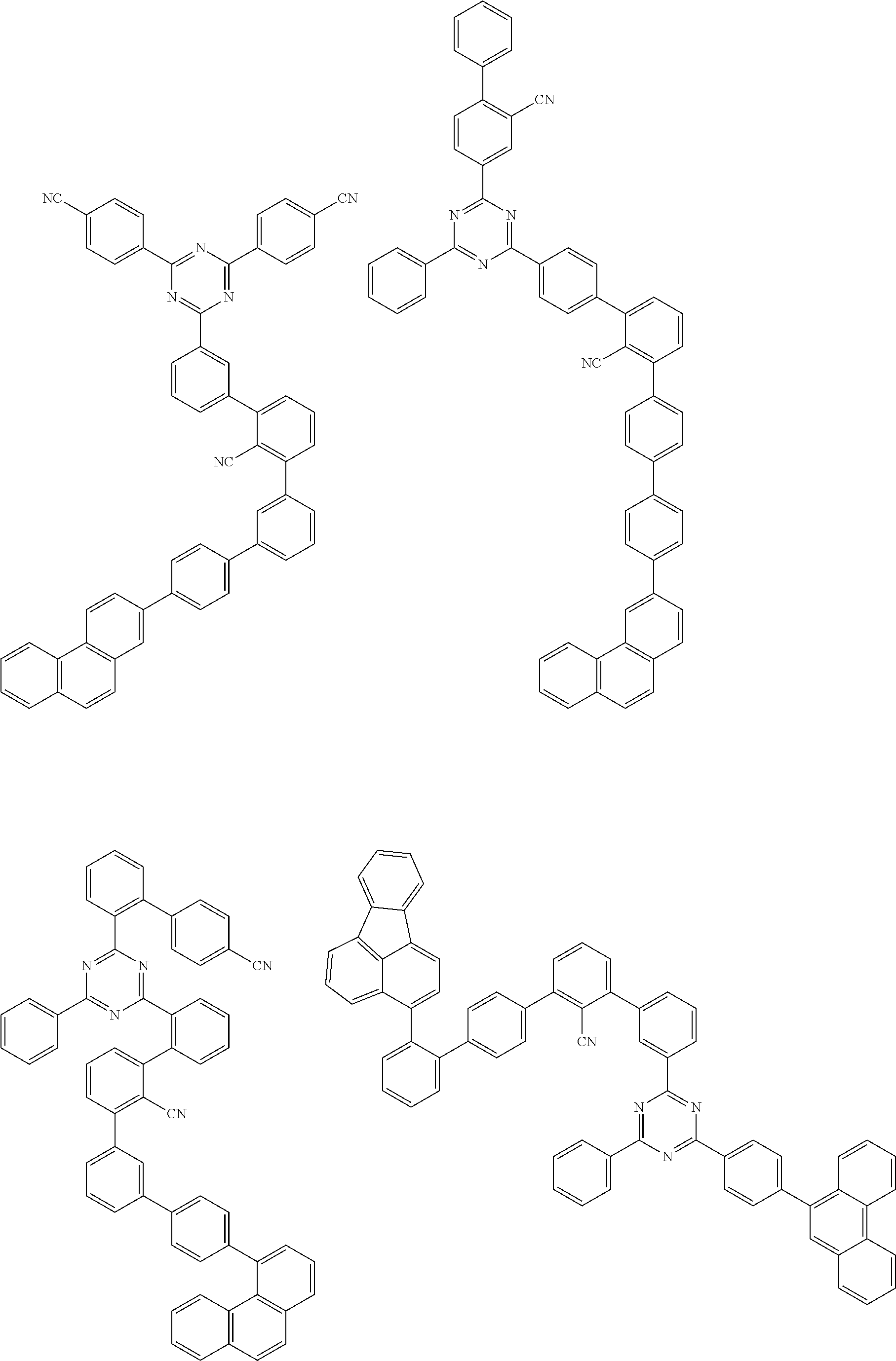 [SMILES: N#Cc1ccc(-c2nc(-c3ccc(C#N)cc3)nc(-c3cccc(-c4cccc(-c5cccc(-c6ccc(-c7ccc8c(ccc9ccccc98)c7)cc6)c5)c4C#N)c3)n2)cc1 ; N#Cc1cc(-c2nc(-c3ccccc3)nc(-c3ccc(-c4cccc(-c5ccc(-c6ccc(-c7ccc8ccc9ccccc9c8c7)cc6)cc5)c4C#N)cc3)n2)ccc1-c1ccccc1 ; N#Cc1ccc(-c2ccccc2-c2nc(-c3ccccc3)nc(-c3ccccc3-c3cccc(-c4cccc(-c5ccc(-c6cccc7ccc8ccccc8c67)cc5)c4)c3C#N)n2)cc1 ; N#Cc1c(-c2ccc(-c3ccccc3-c3ccc4c5c(cccc35)-c3ccccc3-4)cc2)cccc1-c1cccc(-c2nc(-c3ccccc3)nc(-c3ccc(-c4cc5ccccc5c5ccccc45)cc3)n2)c1]

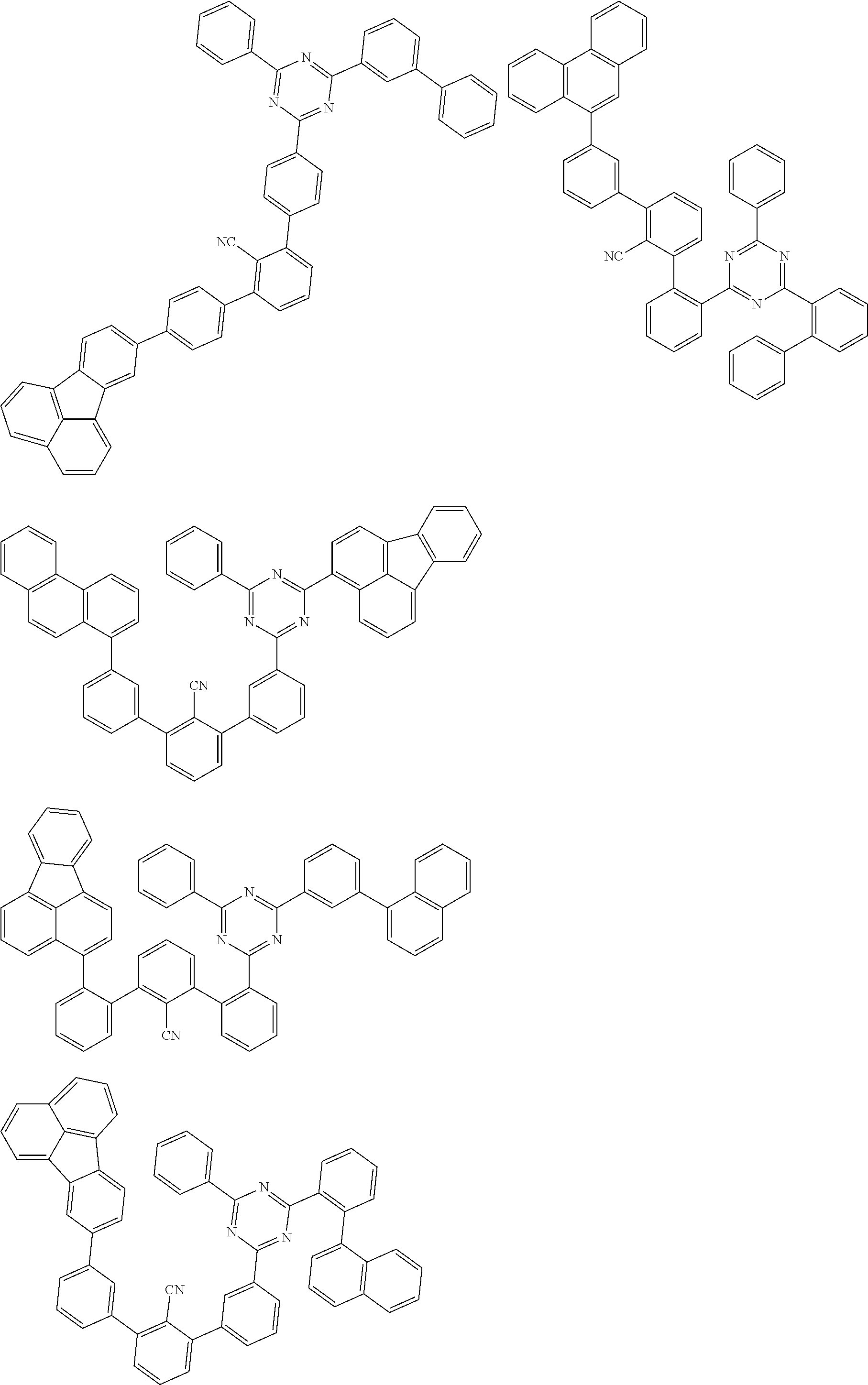

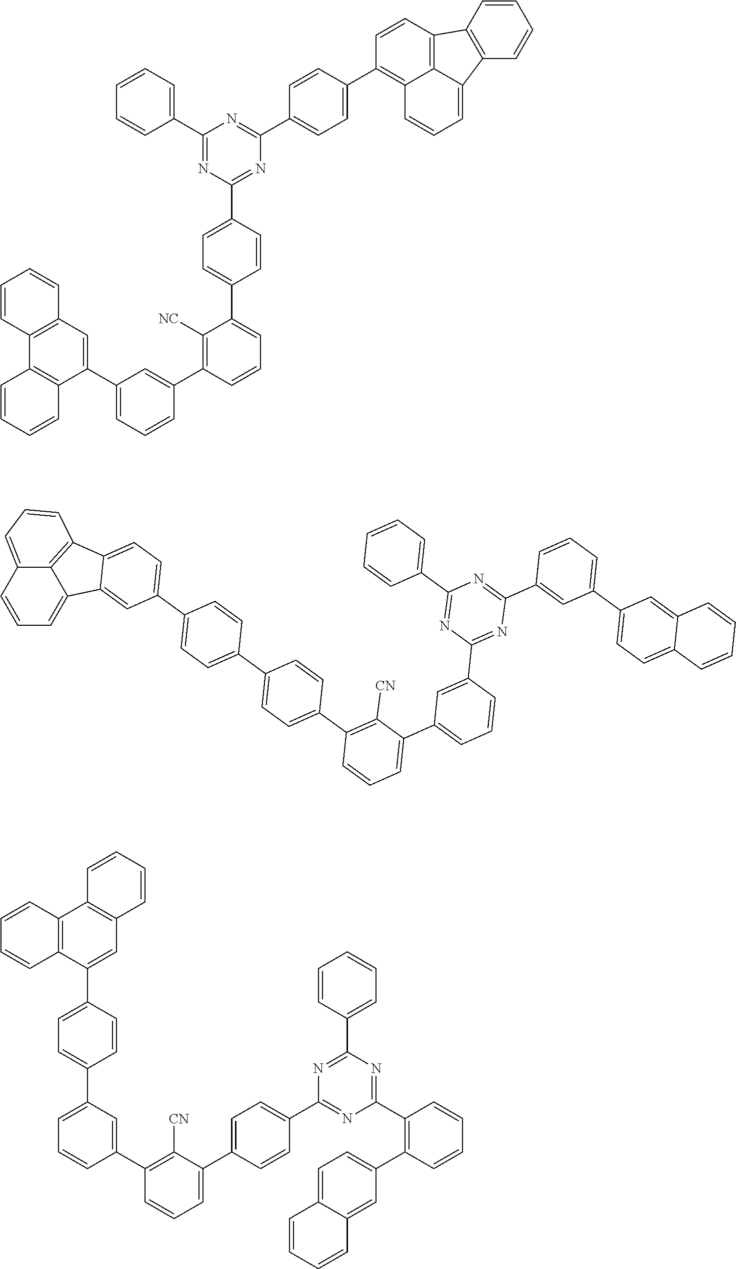 [SMILES: N#Cc1c(-c2ccc(-c3nc(-c4ccccc4)nc(-c4ccc(-c5ccc6c7c(cccc57)-c5ccccc5-6)cc4)n3)cc2)cccc1-c1cccc(-c2cc3ccccc3c3ccccc23)c1 ; N#Cc1c(-c2ccc(-c3ccc(-c4ccc5c(c4)-c4cccc6cccc-5c46)cc3)cc2)cccc1-c1cccc(-c2nc(-c3ccccc3)nc(-c3cccc(-c4ccc5ccccc5c4)c3)n2)c1 ; N#Cc1c(-c2ccc(-c3nc(-c4ccccc4)nc(-c4ccccc4-c4ccc5ccccc5c4)n3)cc2)cccc1-c1cccc(-c2ccc(-c3cc4ccccc4c4ccccc34)cc2)c1]

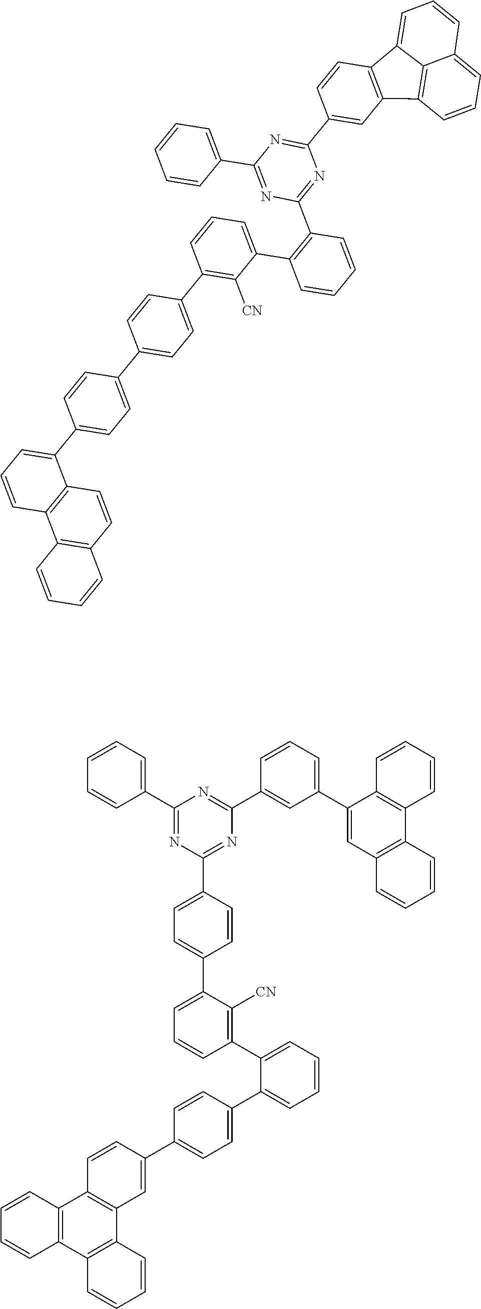 [SMILES: N#Cc1c(-c2ccc(-c3ccc(-c4cccc5c4ccc4ccccc45)cc3)cc2)cccc1-c1ccccc1-c1nc(-c2ccccc2)nc(-c2ccc3c(c2)-c2cccc4cccc-3c24)n1 ; N#Cc1c(-c2ccc(-c3nc(-c4ccccc4)nc(-c4cccc(-c5cc6ccccc6c6ccccc56)c4)n3)cc2)cccc1-c1ccccc1-c1ccc(-c2ccc3c4ccccc4c4ccccc4c3c2)cc1]

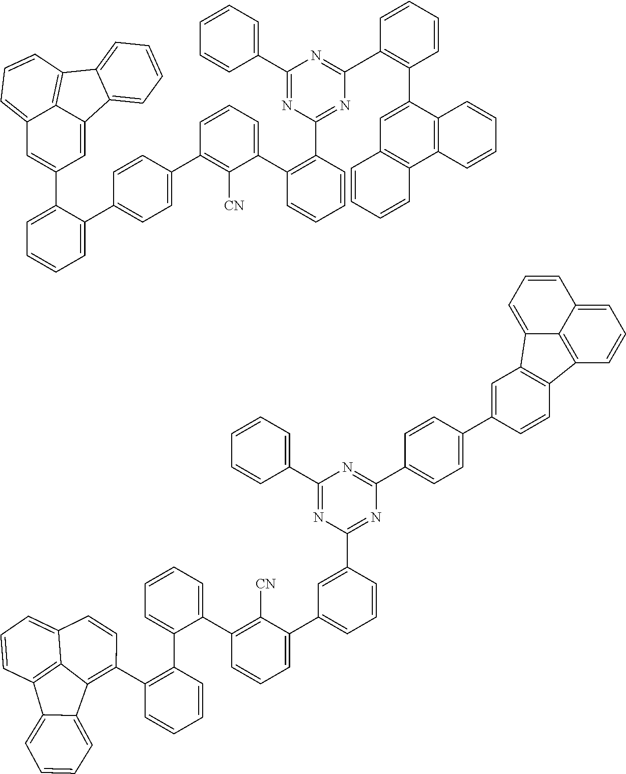 [SMILES: N#Cc1c(-c2ccc(-c3ccccc3-c3cc4c5c(cccc5c3)-c3ccccc3-4)cc2)cccc1-c1ccccc1-c1nc(-c2ccccc2)nc(-c2ccccc2-c2cc3ccccc3c3ccccc23)n1 ; N#Cc1c(-c2cccc(-c3nc(-c4ccccc4)nc(-c4ccc(-c5ccc6c(c5)-c5cccc7cccc-6c57)cc4)n3)c2)cccc1-c1ccccc1-c1ccccc1-c1ccc2cccc3c2c1-c1ccccc1-3]

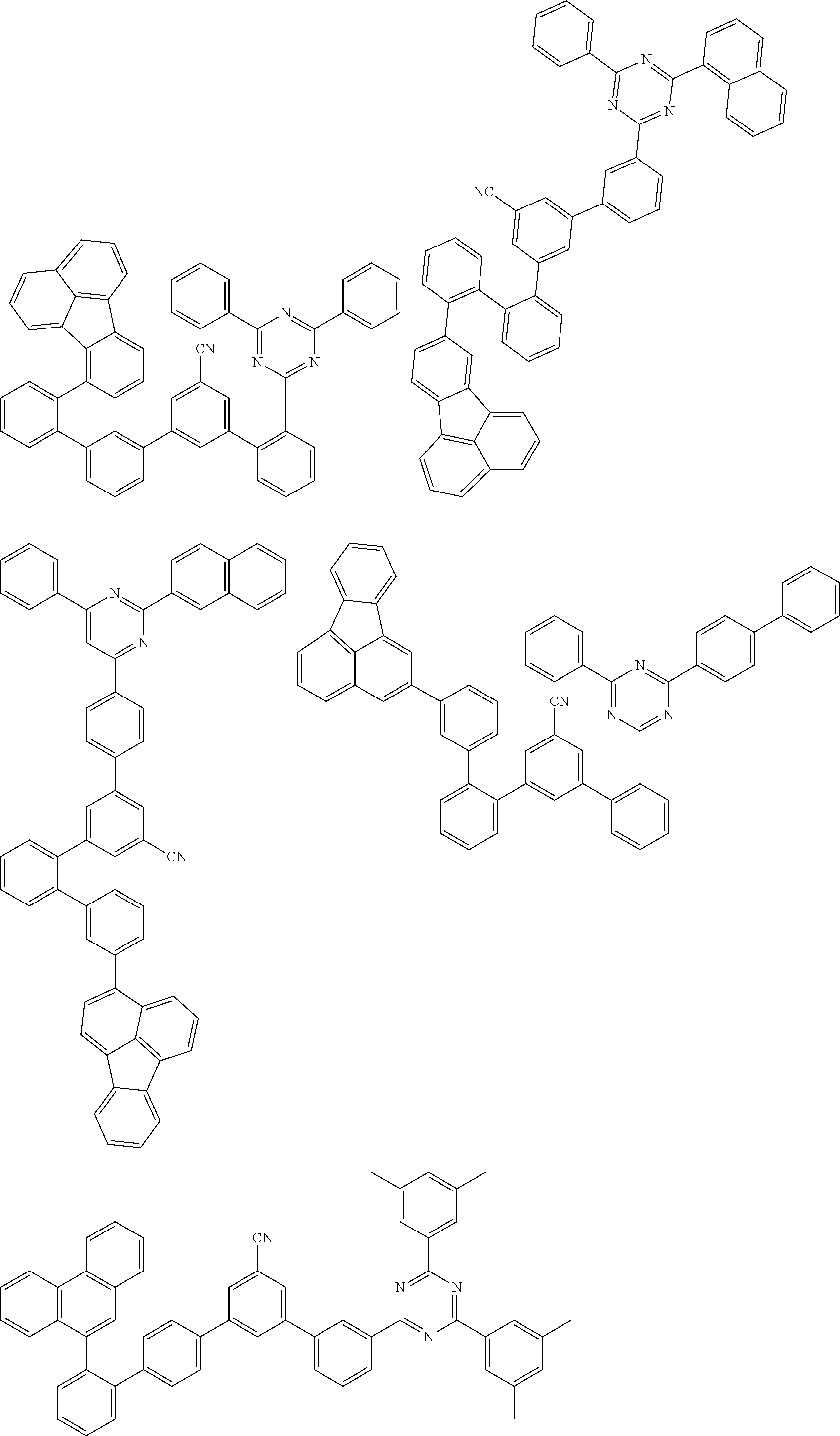

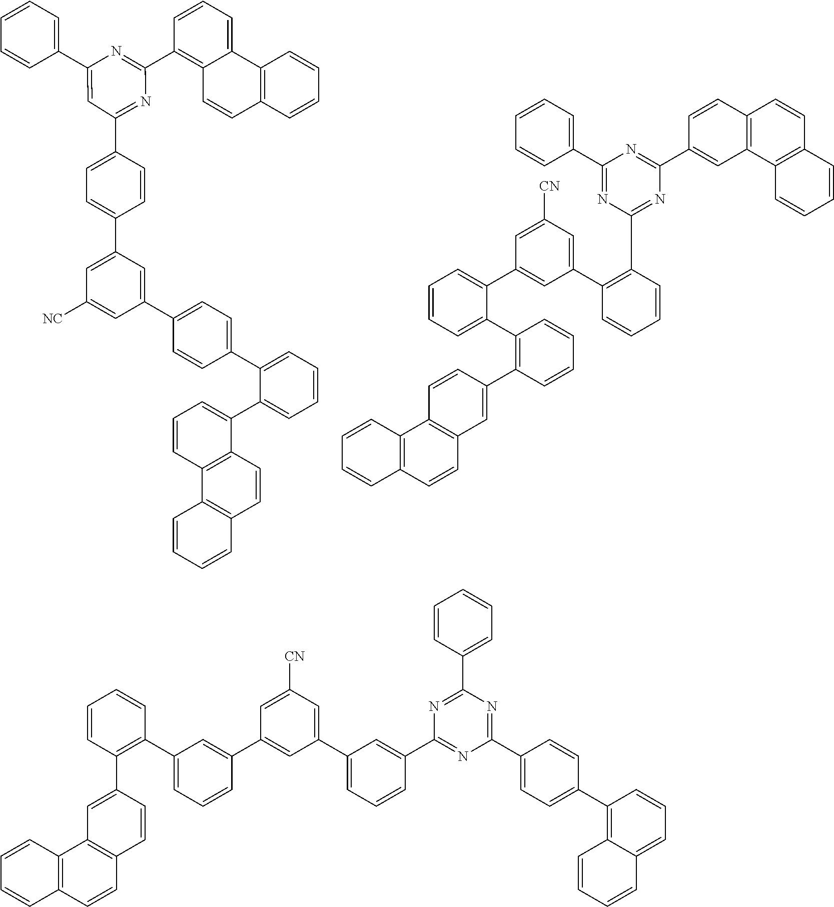

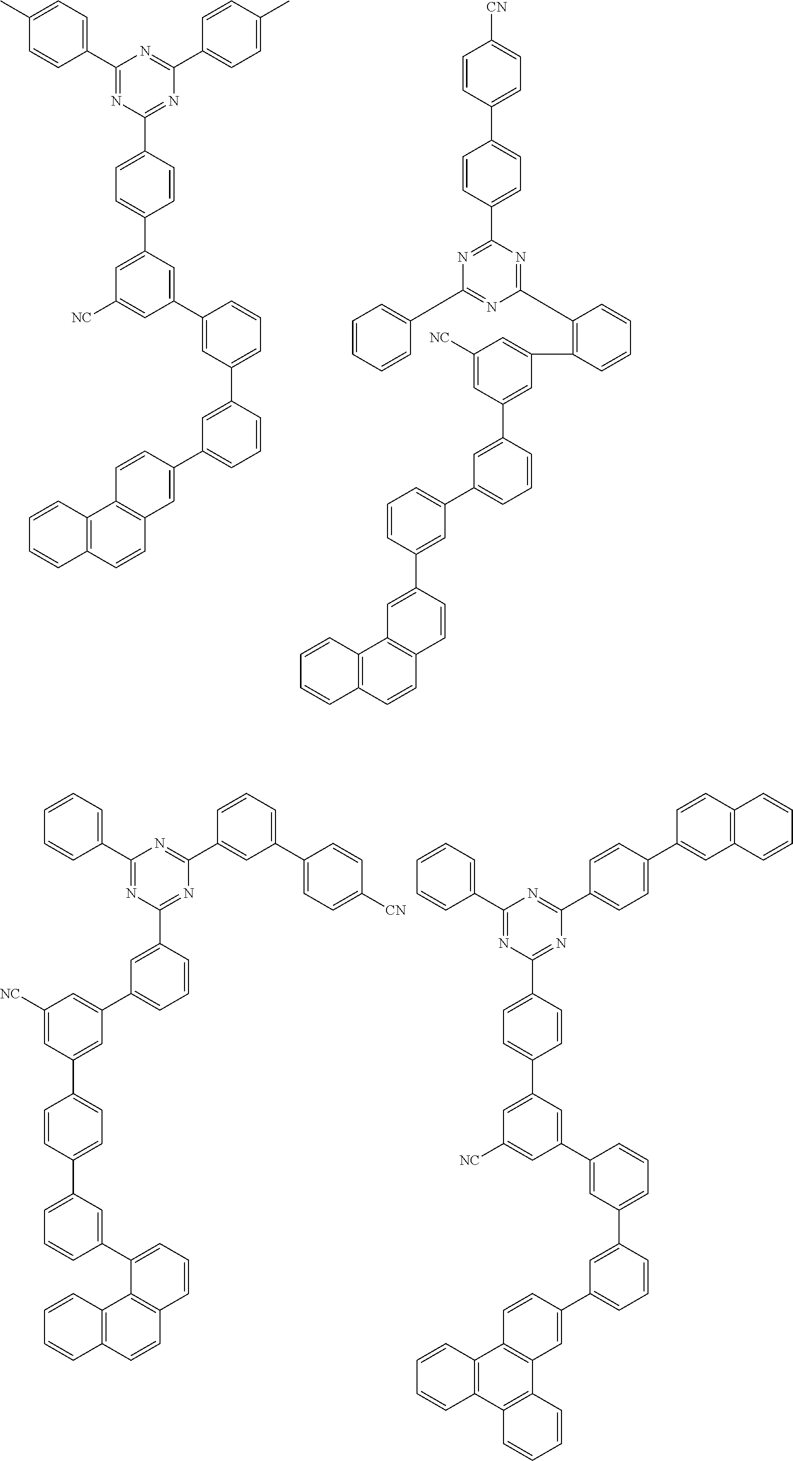

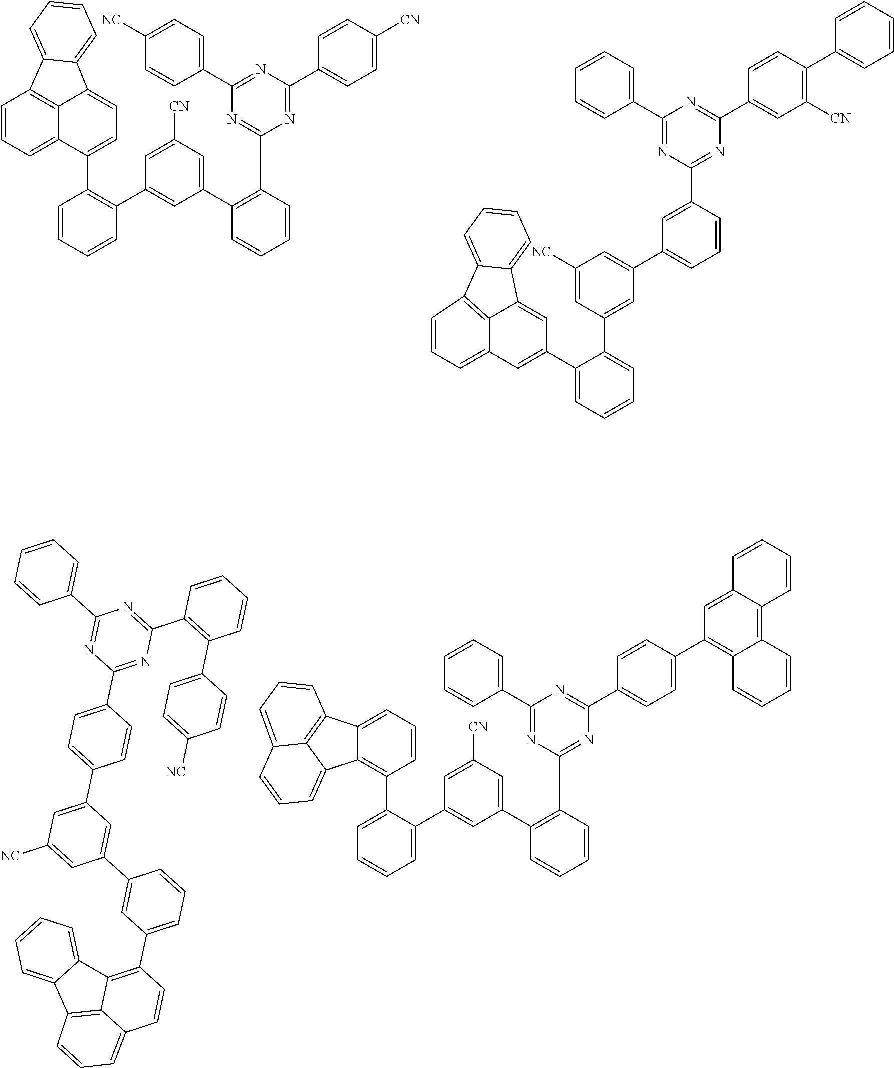

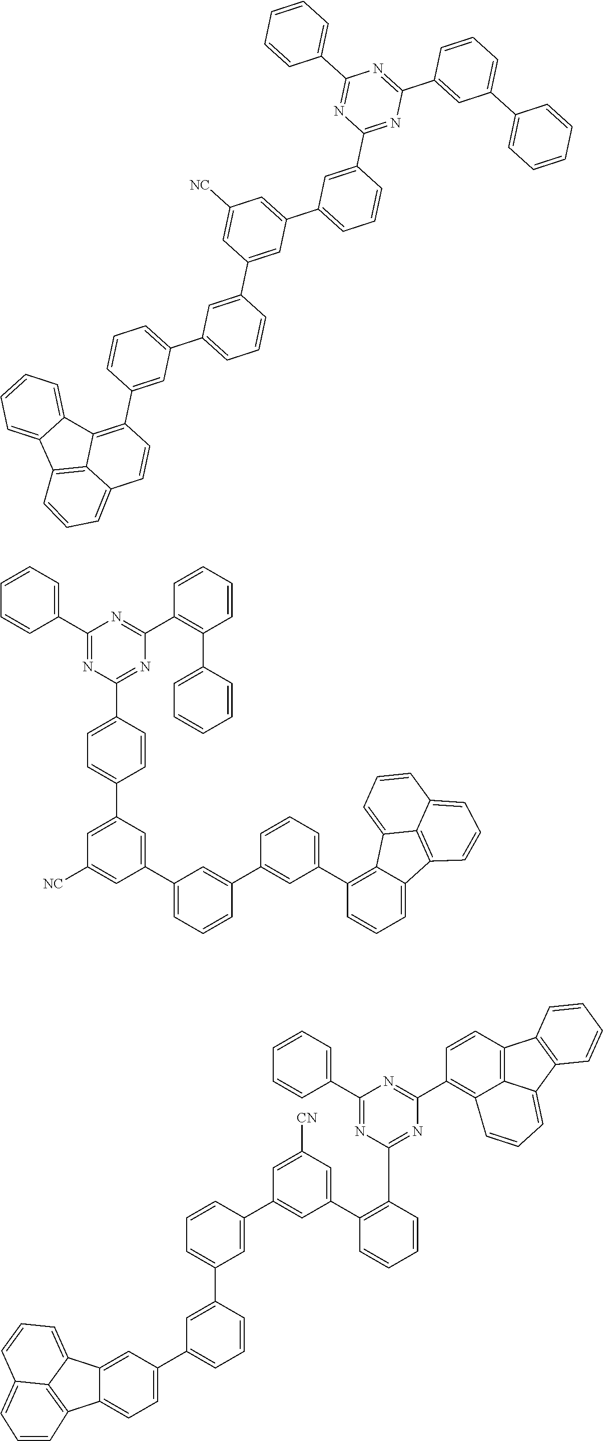 [SMILES: N#Cc1cc(-c2cccc(-c3cccc(-c4ccc5cccc6c5c4-c4ccccc4-6)c3)c2)cc(-c2cccc(-c3nc(-c4ccccc4)nc(-c4cccc(-c5ccccc5)c4)n3)c2)c1 ; N#Cc1cc(-c2ccc(-c3nc(-c4ccccc4)nc(-c4ccccc4-c4ccccc4)n3)cc2)cc(-c2cccc(-c3cccc(-c4cccc5c4-c4cccc6cccc-5c46)c3)c2)c1 ; N#Cc1cc(-c2cccc(-c3cccc(-c4ccc5c(c4)-c4cccc6cccc-5c46)c3)c2)cc(-c2ccccc2-c2nc(-c3ccccc3)nc(-c3ccc4c5c(cccc35)-c3ccccc3-4)n2)c1]

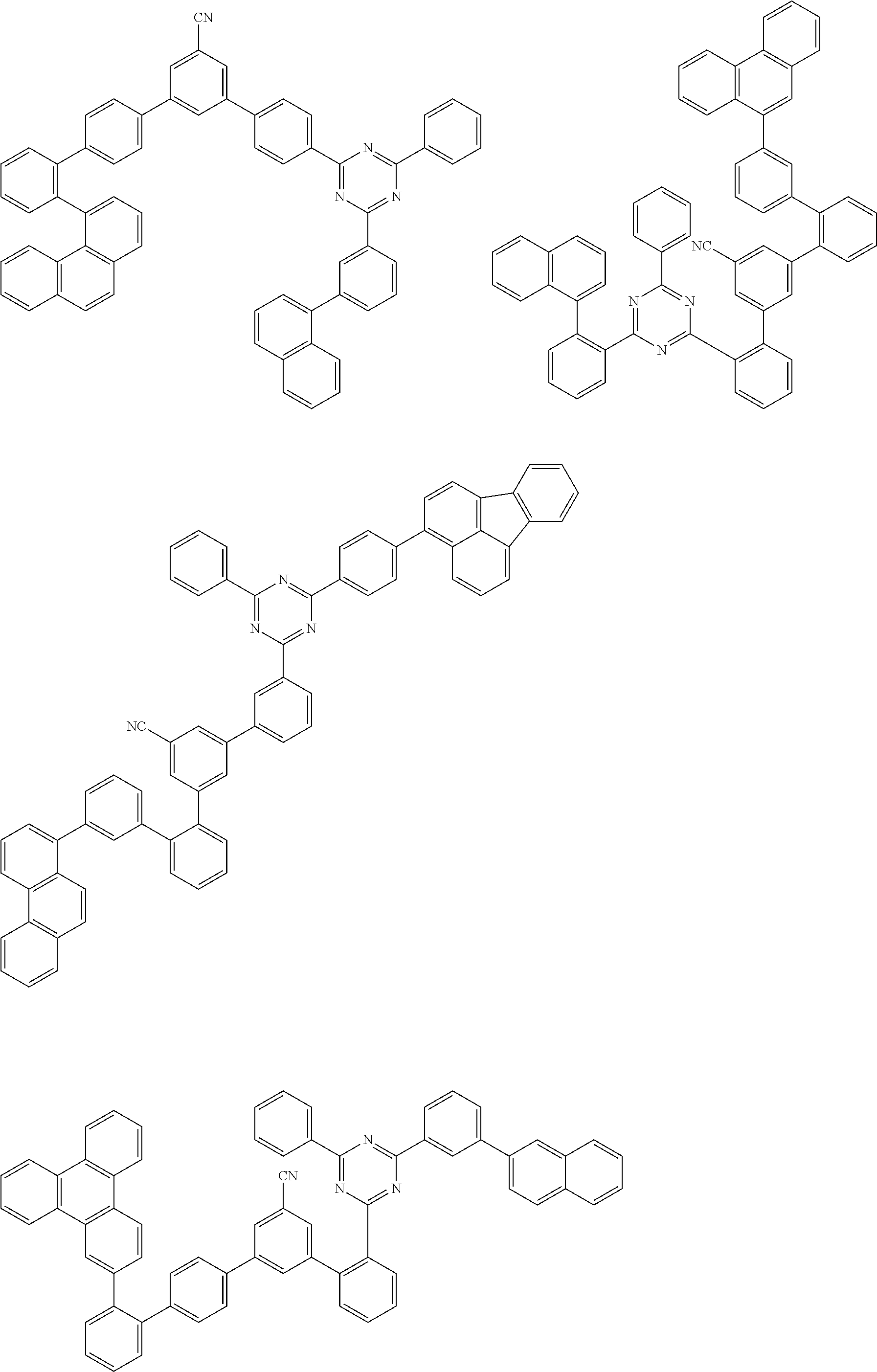 [SMILES: N#Cc1cc(-c2ccc(-c3nc(-c4ccccc4)nc(-c4cccc(-c5cccc6ccccc56)c4)n3)cc2)cc(-c2ccc(-c3ccccc3-c3cccc4ccc5ccccc5c34)cc2)c1 ; N#Cc1cc(-c2ccccc2-c2cccc(-c3cc4ccccc4c4ccccc34)c2)cc(-c2ccccc2-c2nc(-c3ccccc3)nc(-c3ccccc3-c3cccc4ccccc34)n2)c1 ; N#Cc1cc(-c2cccc(-c3nc(-c4ccccc4)nc(-c4ccc(-c5ccc6c7c(cccc57)-c5ccccc5-6)cc4)n3)c2)cc(-c2ccccc2-c2cccc(-c3cccc4c3ccc3ccccc34)c2)c1 ; N#Cc1cc(-c2ccc(-c3ccccc3-c3ccc4c5ccccc5c5ccccc5c4c3)cc2)cc(-c2ccccc2-c2nc(-c3ccccc3)nc(-c3cccc(-c4ccc5ccccc5c4)c3)n2)c1]

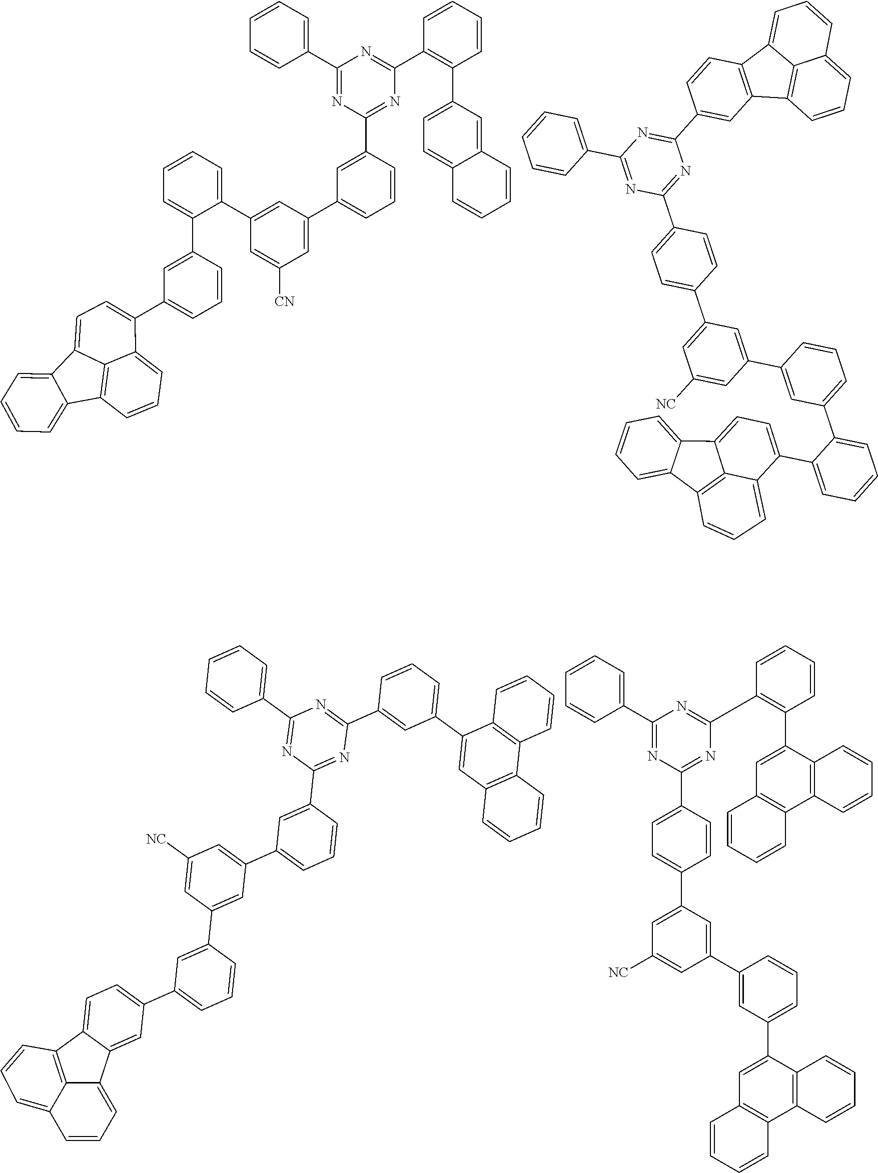

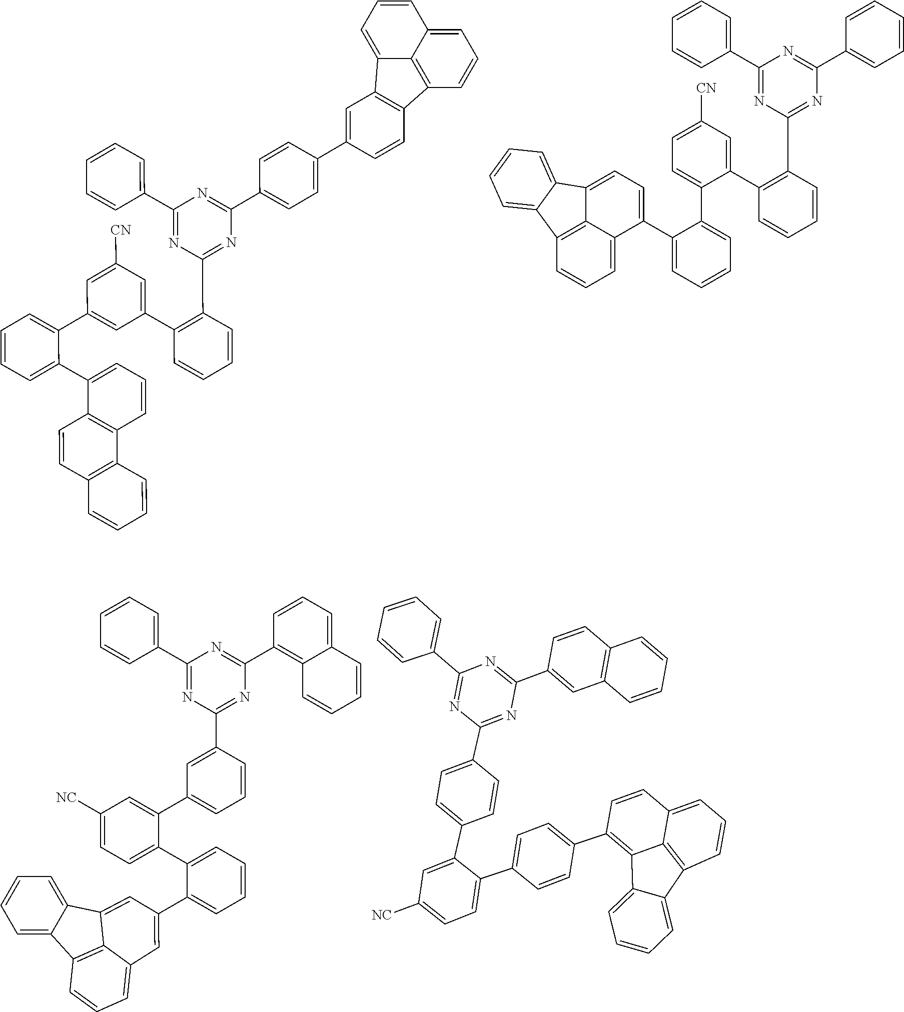

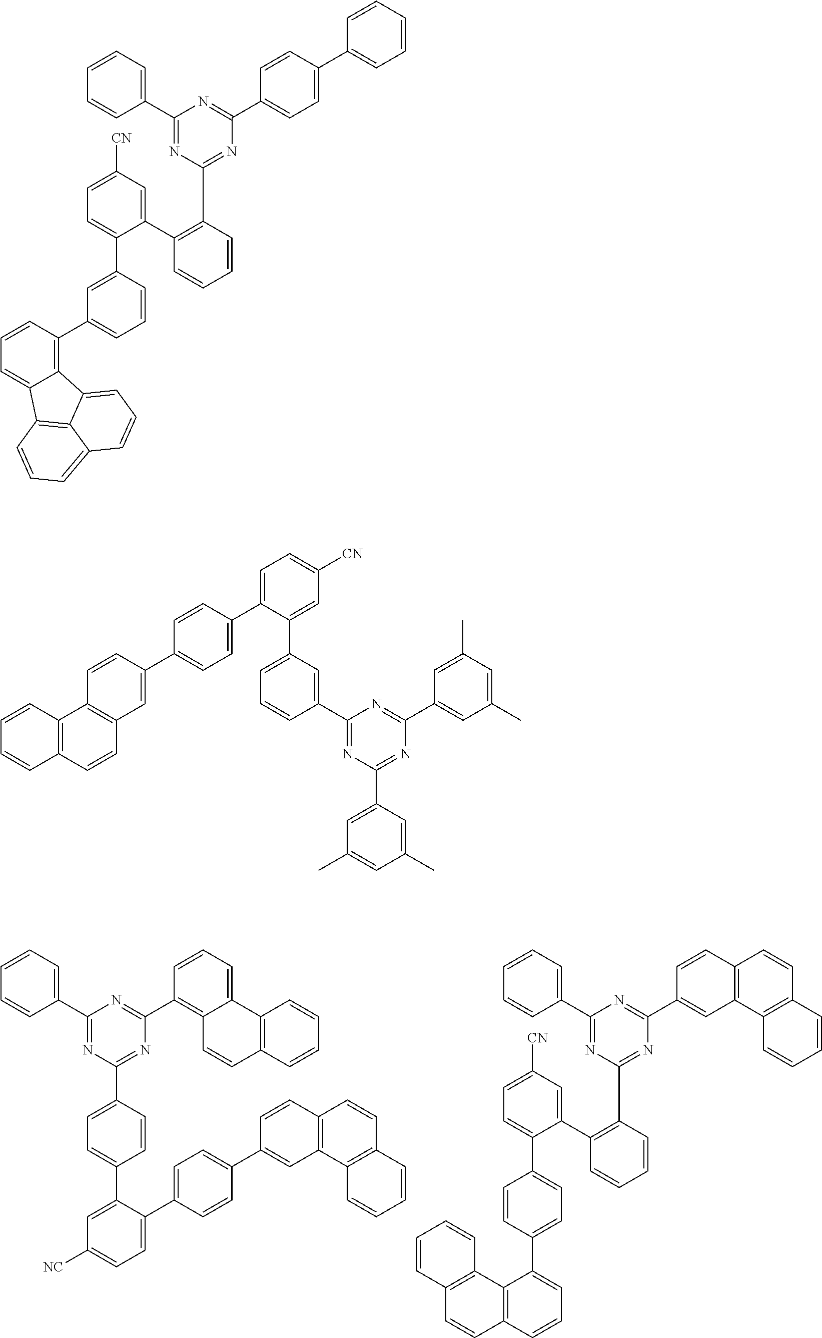

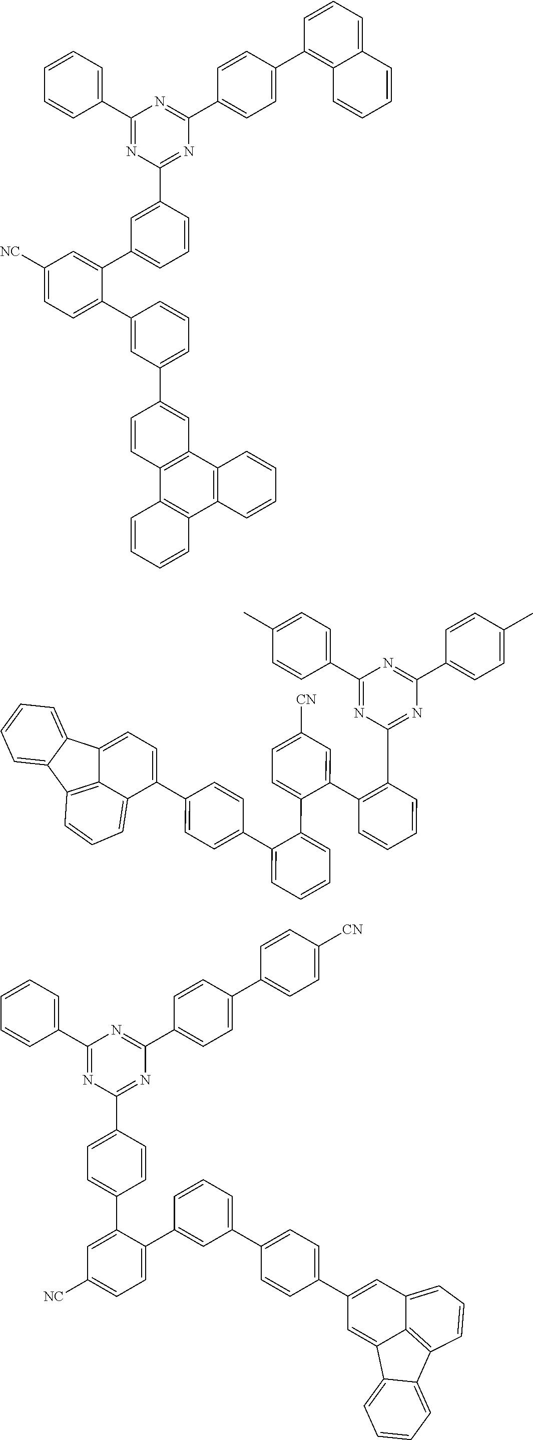

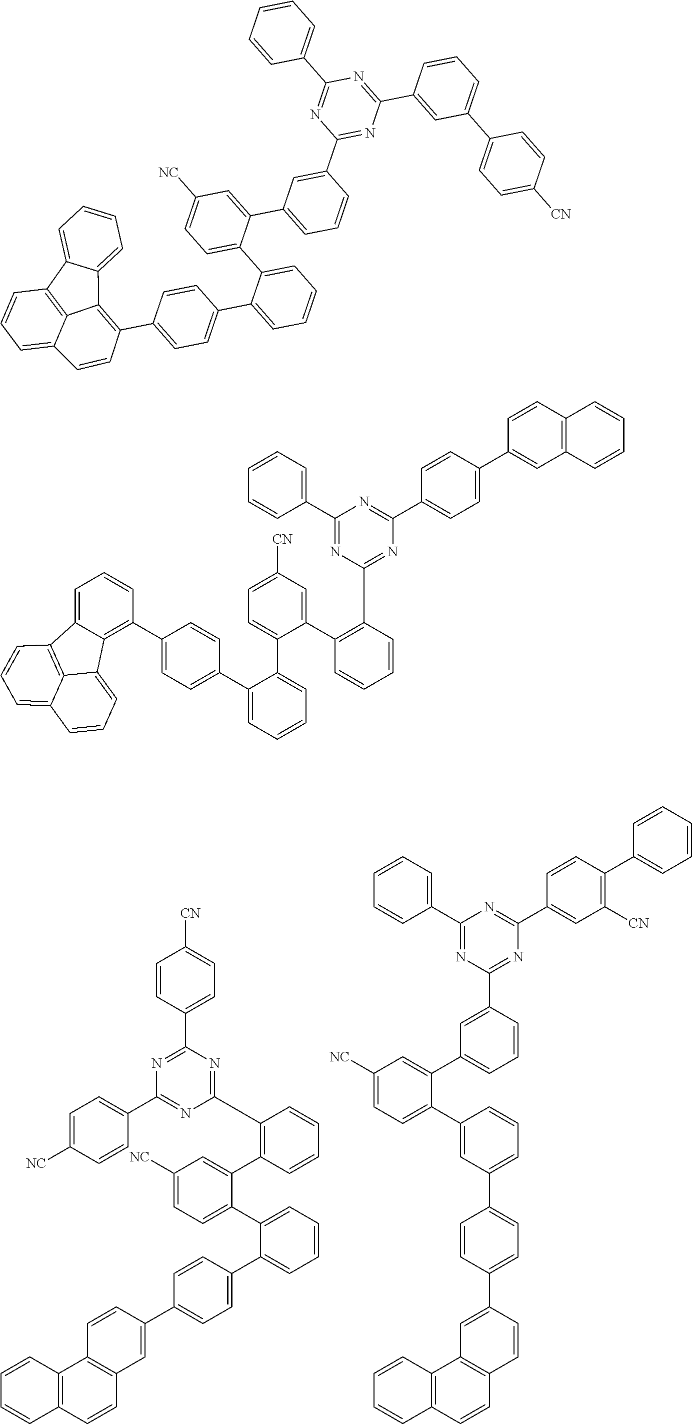 [SMILES: N#Cc1ccc(-c2cccc(-c3nc(-c4ccccc4)nc(-c4cccc(-c5cc(C#N)ccc5-c5ccccc5-c5ccc(-c6ccc7cccc8c7c6-c6ccccc6-8)cc5)c4)n3)c2)cc1 ; N#Cc1ccc(-c2ccccc2-c2ccc(-c3cccc4c3-c3cccc5cccc-4c35)cc2)c(-c2ccccc2-c2nc(-c3ccccc3)nc(-c3ccc(-c4ccc5ccccc5c4)cc3)n2)c1 ; N#Cc1ccc(-c2nc(-c3ccc(C#N)cc3)nc(-c3ccccc3-c3cc(C#N)ccc3-c3ccccc3-c3ccc(-c4ccc5c(ccc6ccccc65)c4)cc3)n2)cc1 ; N#Cc1ccc(-c2cccc(-c3ccc(-c4ccc5ccc6ccccc6c5c4)cc3)c2)c(-c2cccc(-c3nc(-c4ccccc4)nc(-c4ccc(-c5ccccc5)c(C#N)c4)n3)c2)c1]

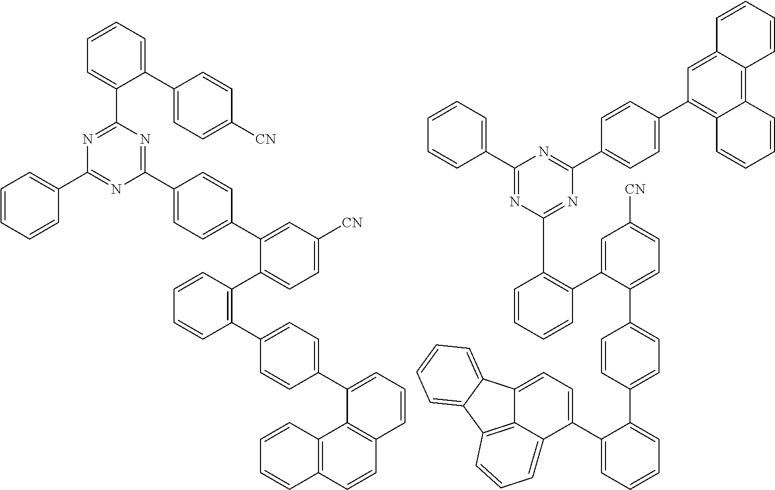

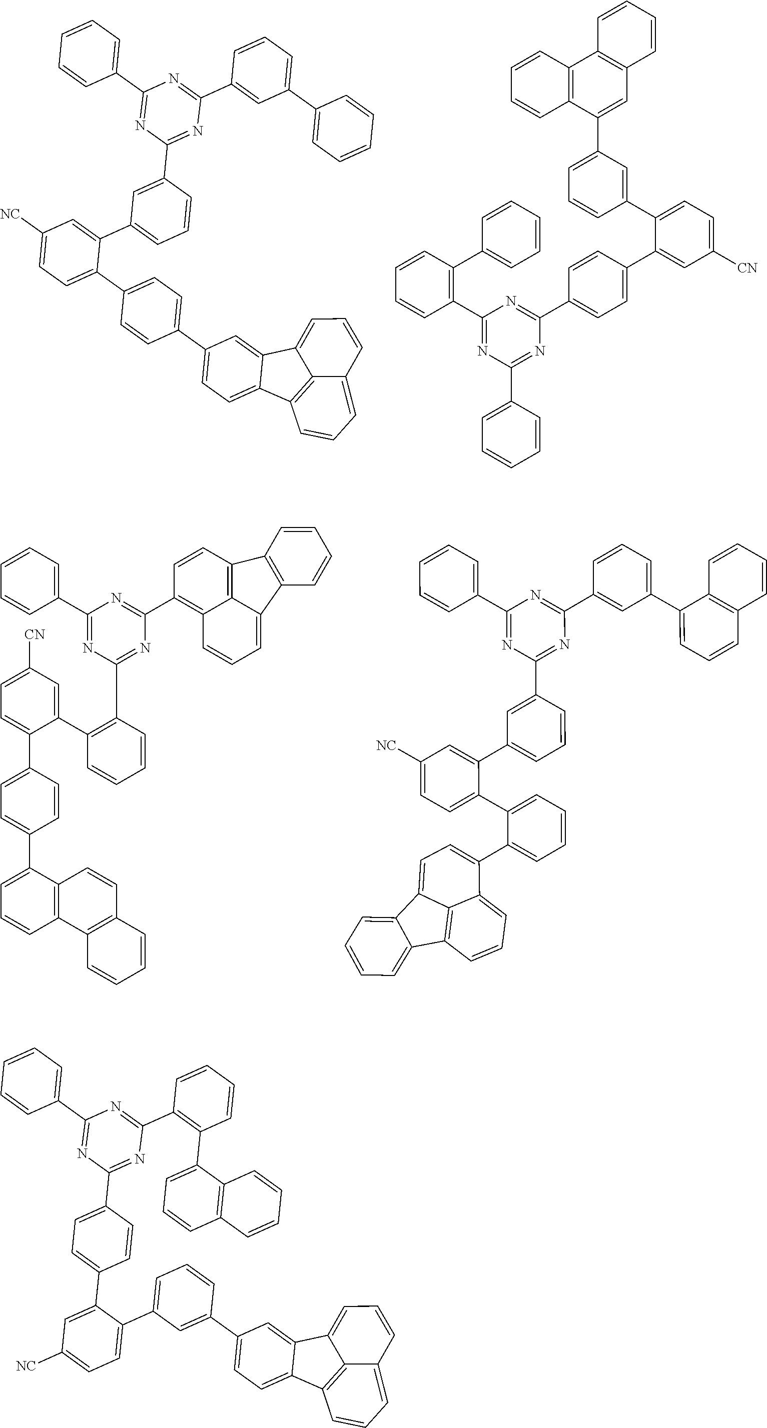

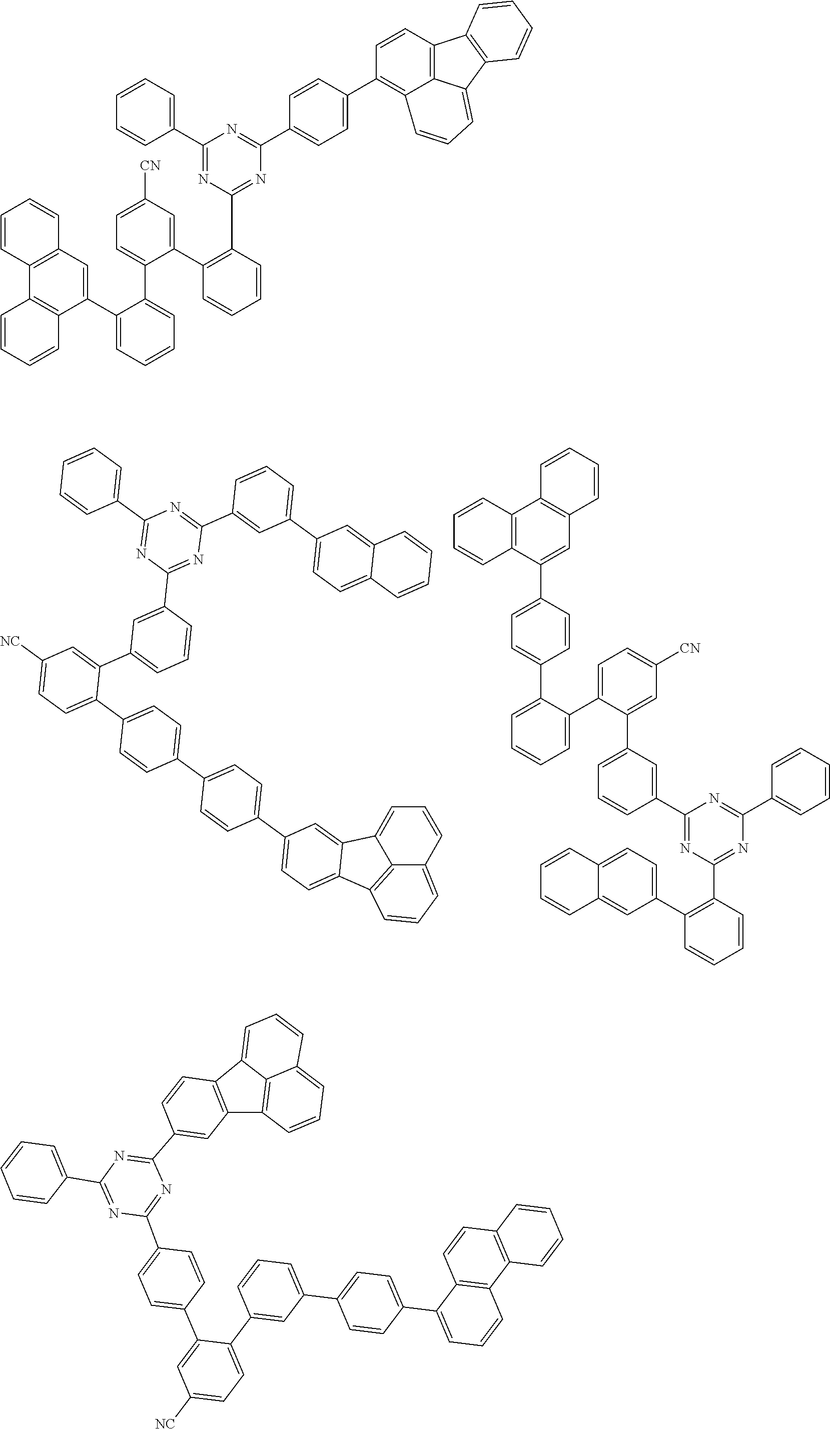

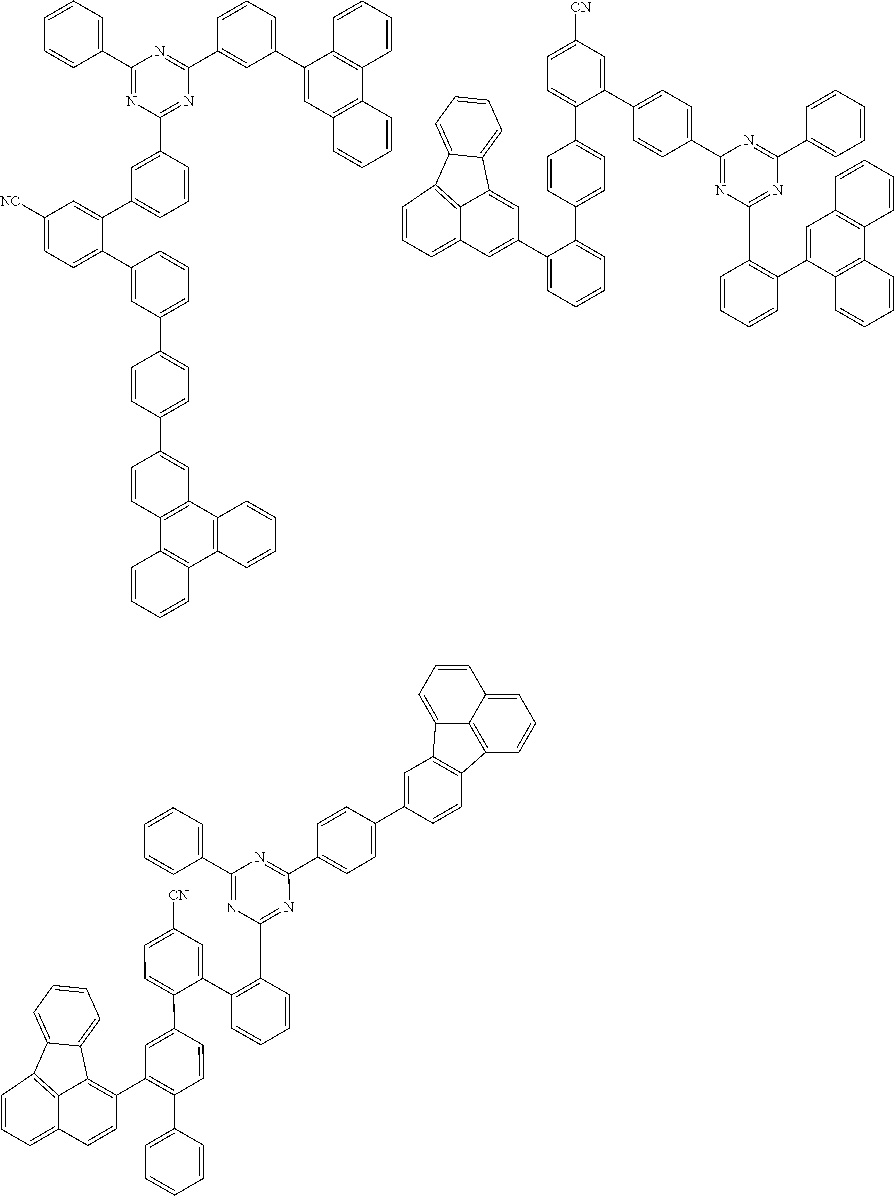

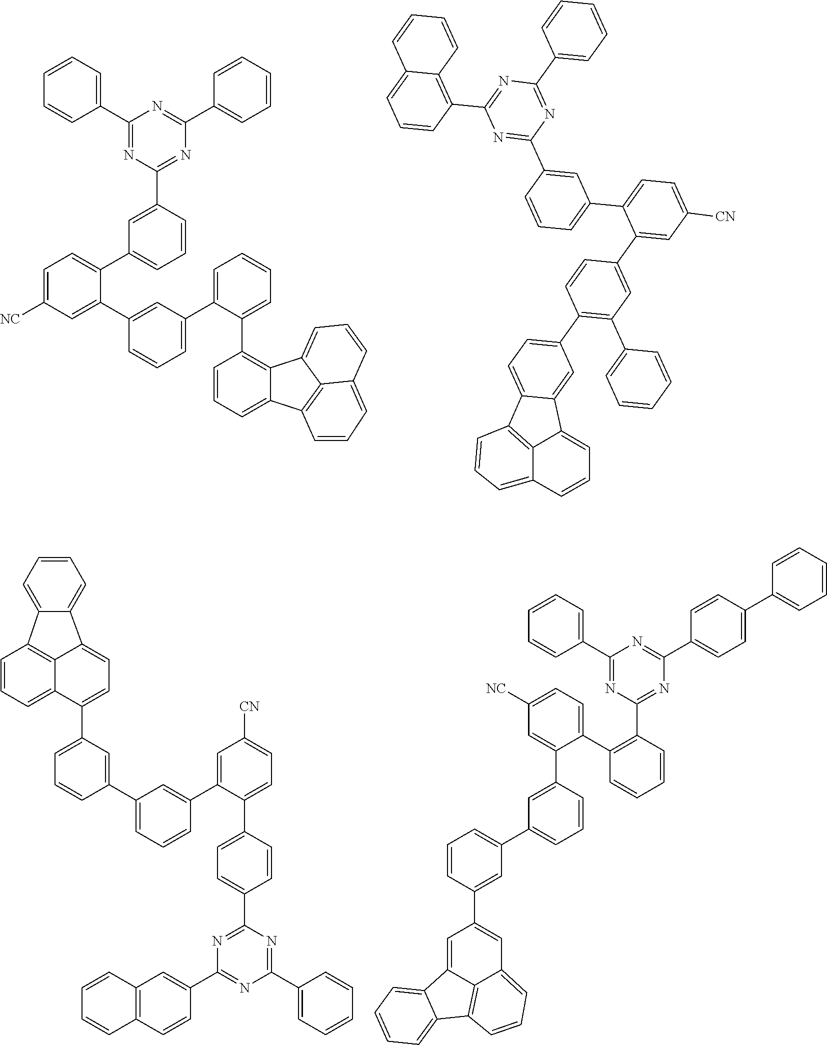

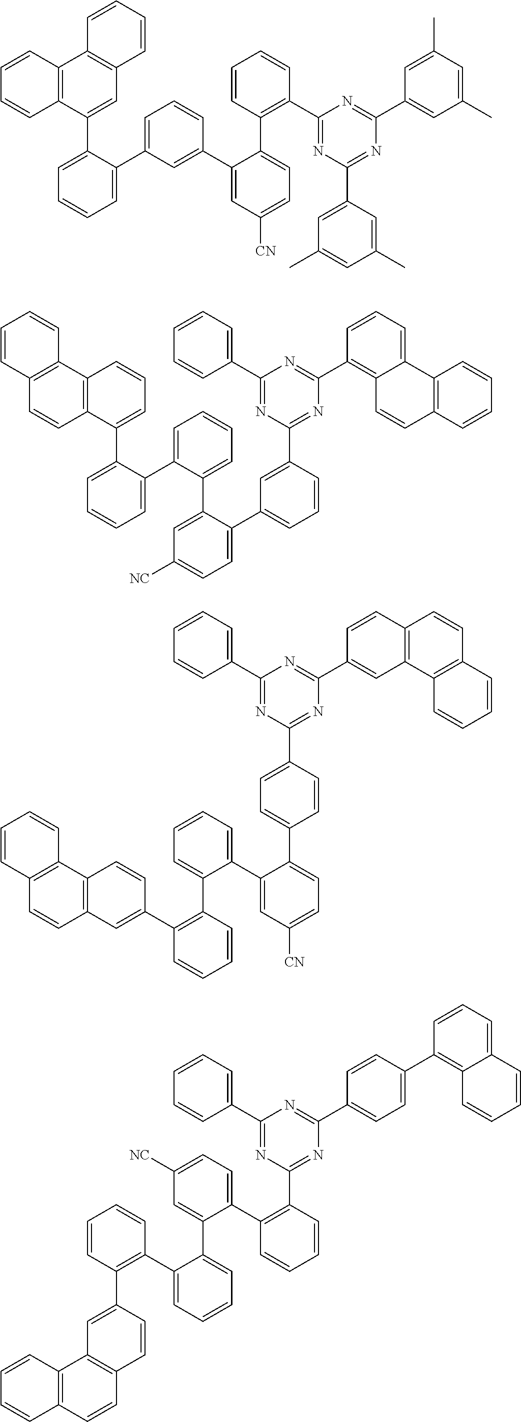

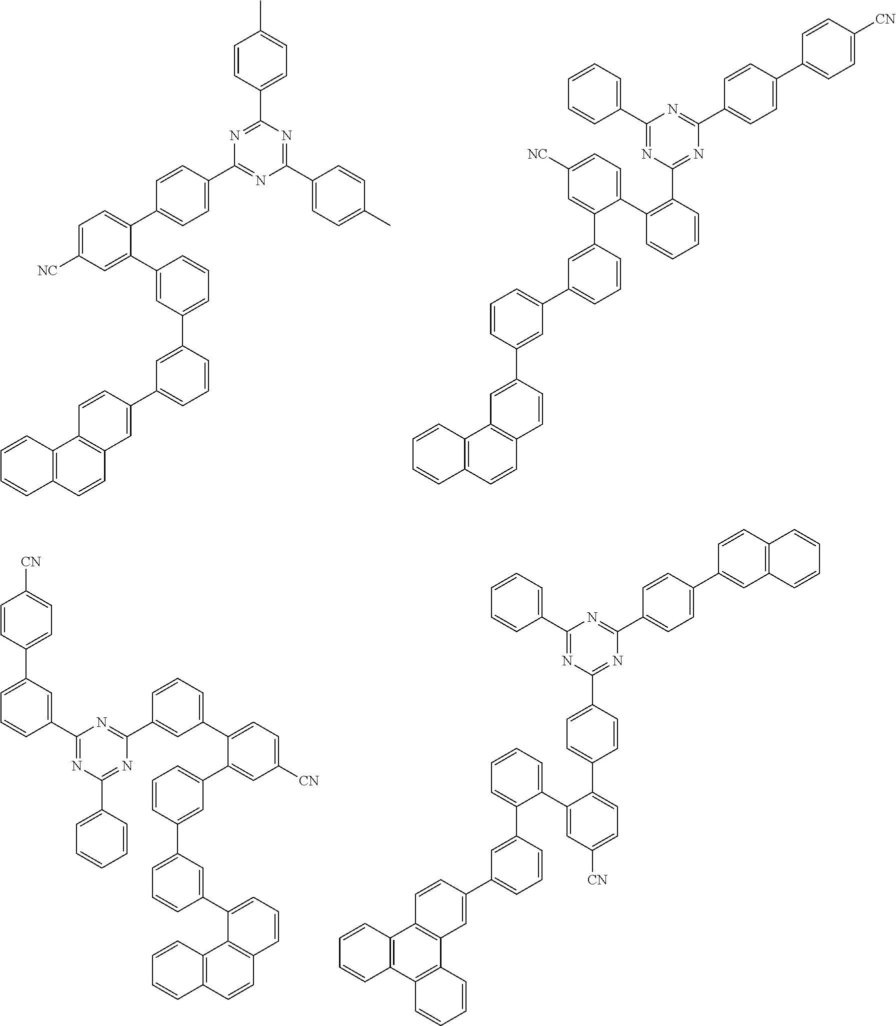 [SMILES: Cc1ccc(-c2nc(-c3ccc(C)cc3)nc(-c3ccc(-c4ccc(C#N)cc4-c4cccc(-c5cccc(-c6ccc7c(ccc8ccccc87)c6)c5)c4)cc3)n2)cc1 ; N#Cc1ccc(-c2ccc(-c3nc(-c4ccccc4)nc(-c4ccccc4-c4ccc(C#N)cc4-c4cccc(-c5cccc(-c6ccc7ccc8ccccc8c7c6)c5)c4)n3)cc2)cc1 ; N#Cc1ccc(-c2cccc(-c3nc(-c4ccccc4)nc(-c4cccc(-c5ccc(C#N)cc5-c5cccc(-c6cccc(-c7cccc8ccc9ccccc9c78)c6)c5)c4)n3)c2)cc1 ; N#Cc1ccc(-c2ccc(-c3nc(-c4ccccc4)nc(-c4ccc(-c5ccc6ccccc6c5)cc4)n3)cc2)c(-c2ccccc2-c2cccc(-c3ccc4c5ccccc5c5ccccc5c4c3)c2)c1]

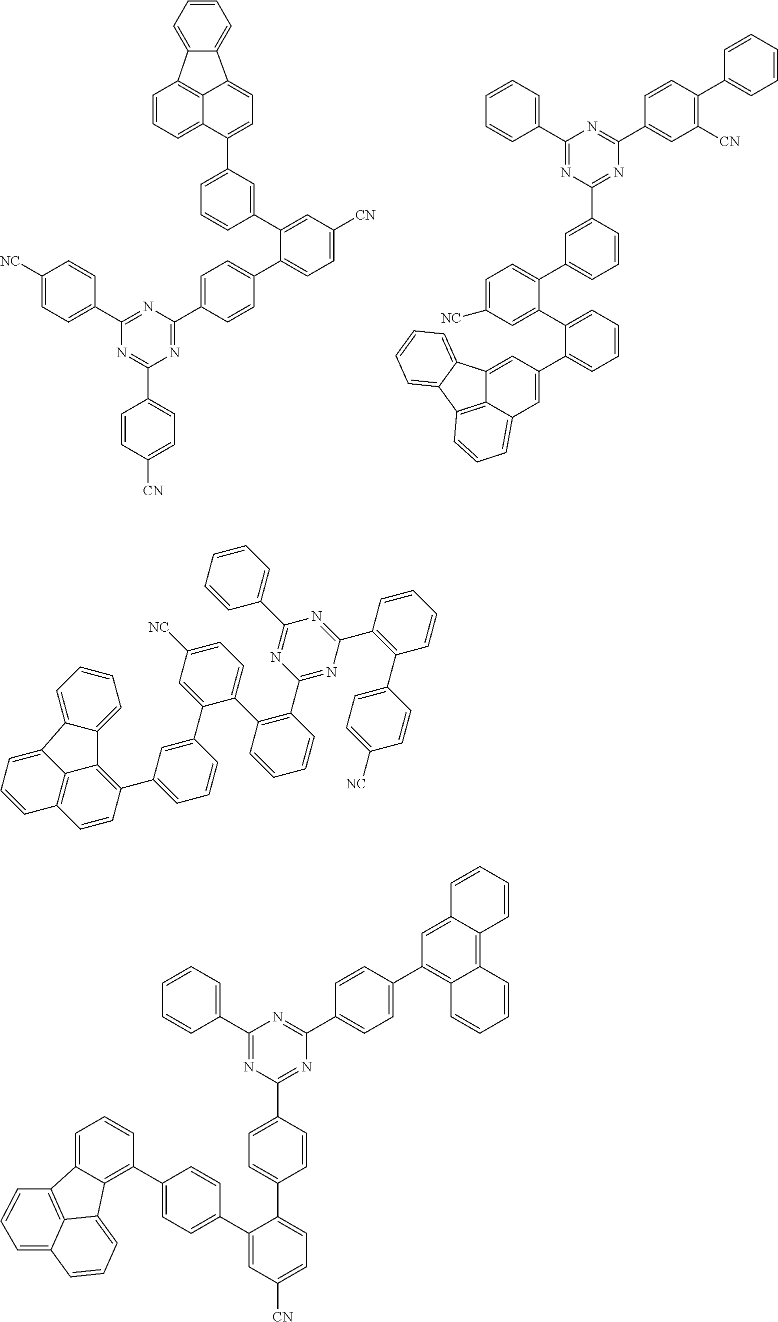 [SMILES: N#Cc1ccc(-c2nc(-c3ccc(C#N)cc3)nc(-c3ccc(-c4ccc(C#N)cc4-c4cccc(-c5ccc6c7c(cccc57)-c5ccccc5-6)c4)cc3)n2)cc1 ; N#Cc1ccc(-c2cccc(-c3nc(-c4ccccc4)nc(-c4ccc(-c5ccccc5)c(C#N)c4)n3)c2)c(-c2ccccc2-c2cc3c4c(cccc4c2)-c2ccccc2-3)c1 ; N#Cc1ccc(-c2ccccc2-c2nc(-c3ccccc3)nc(-c3ccccc3-c3ccc(C#N)cc3-c3cccc(-c4ccc5cccc6c5c4-c4ccccc4-6)c3)n2)cc1 ; N#Cc1ccc(-c2ccc(-c3nc(-c4ccccc4)nc(-c4ccc(-c5cc6ccccc6c6ccccc56)cc4)n3)cc2)c(-c2ccc(-c3cccc4c3-c3cccc5cccc-4c35)cc2)c1]

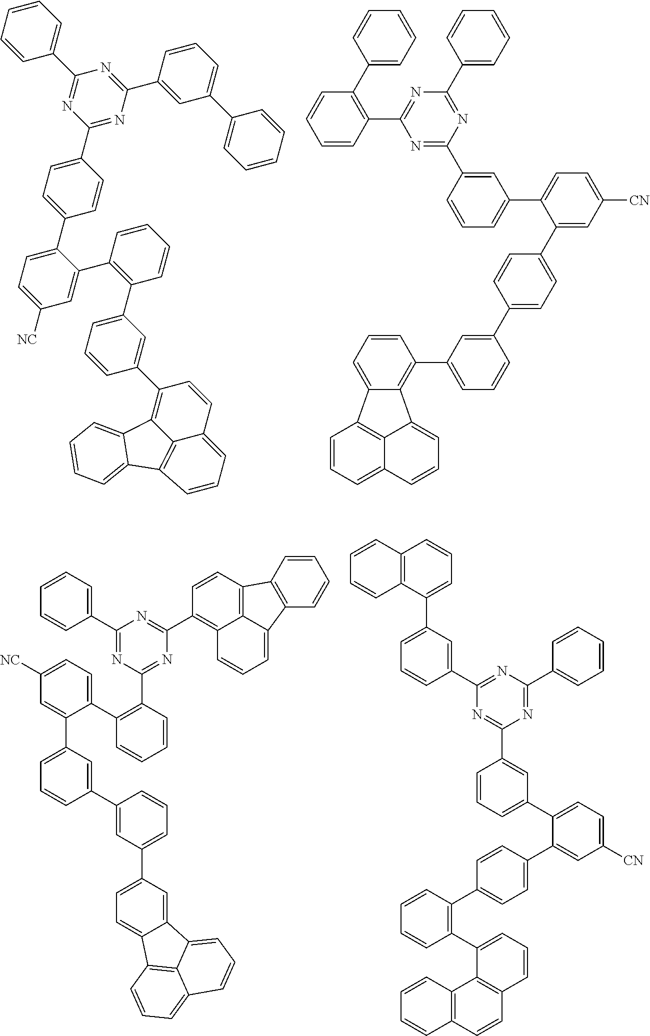

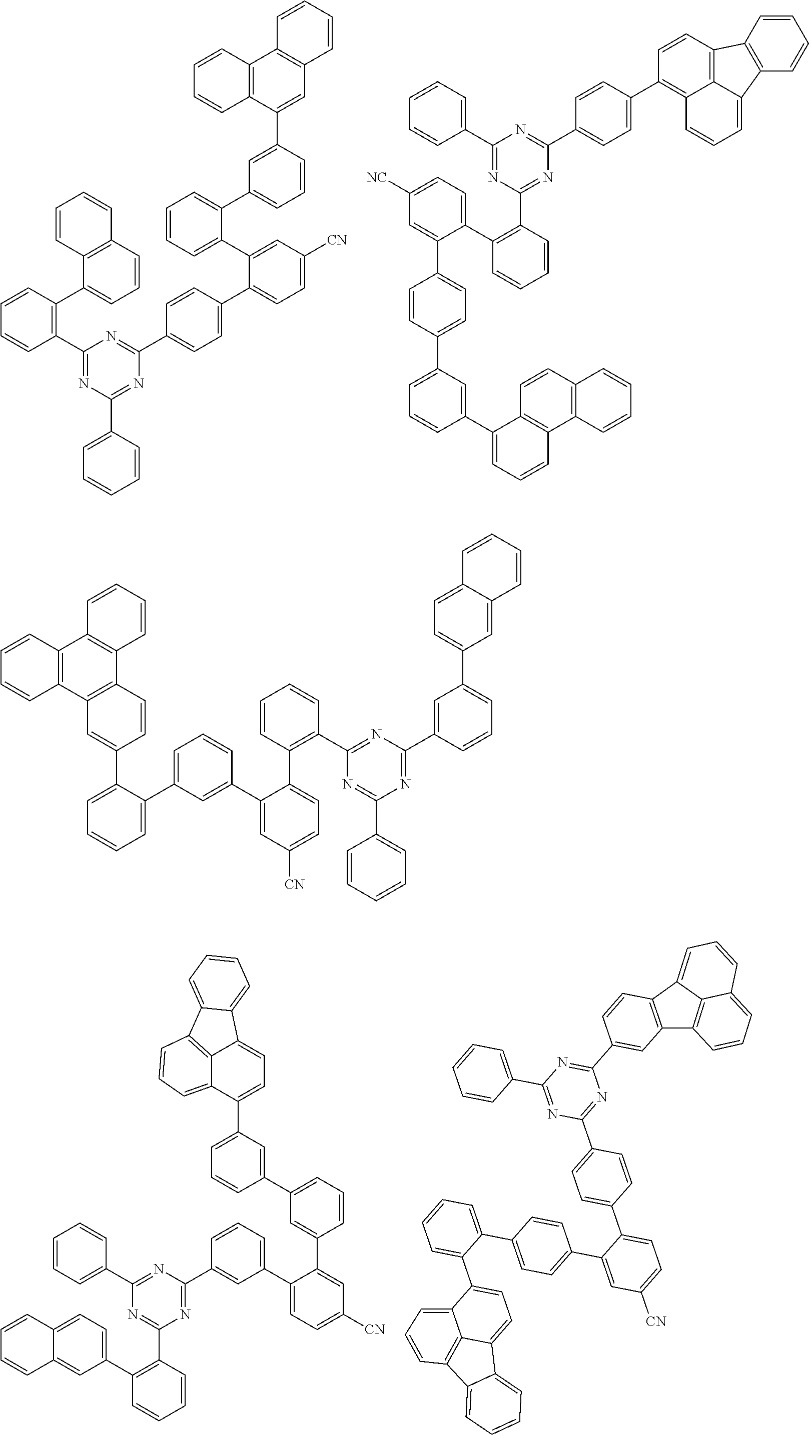

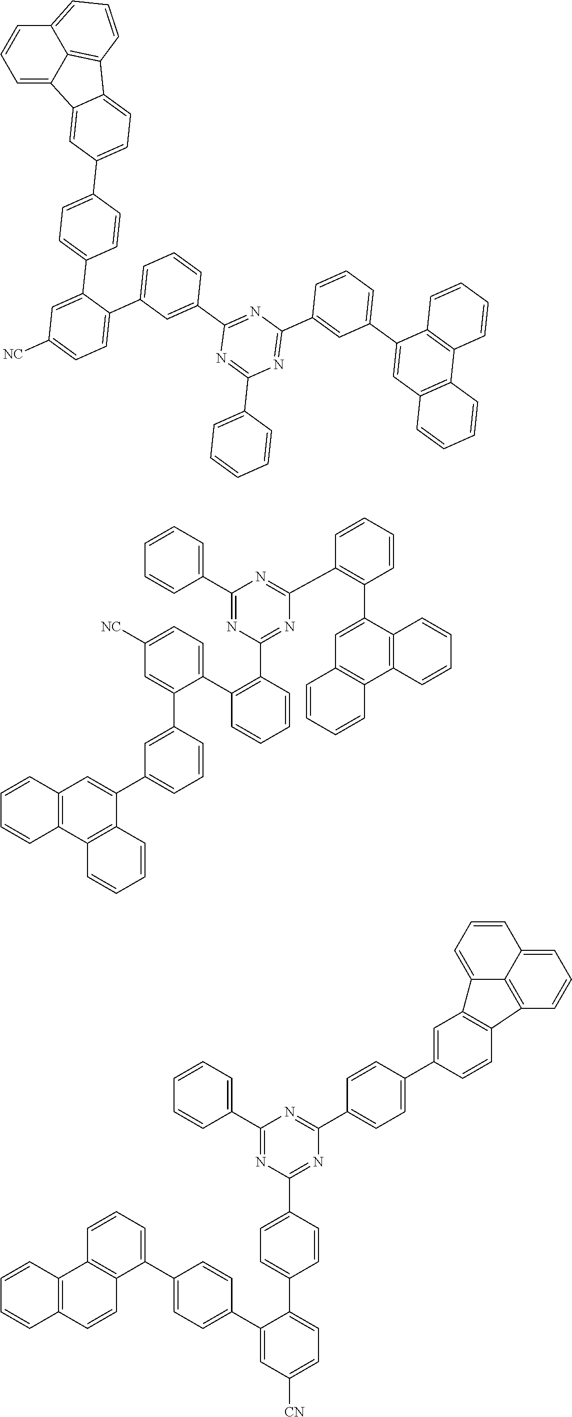 [SMILES: N#Cc1ccc(-c2cccc(-c3nc(-c4ccccc4)nc(-c4cccc(-c5cc6ccccc6c6ccccc56)c4)n3)c2)c(-c2ccc(-c3ccc4c(c3)-c3cccc5cccc-4c35)cc2)c1 ; N#Cc1ccc(-c2ccccc2-c2nc(-c3ccccc3)nc(-c3ccccc3-c3cc4ccccc4c4ccccc34)n2)c(-c2cccc(-c3cc4ccccc4c4ccccc34)c2)c1 ; N#Cc1ccc(-c2ccc(-c3nc(-c4ccccc4)nc(-c4ccc(-c5ccc6c(c5)-c5cccc7cccc-6c57)cc4)n3)cc2)c(-c2ccc(-c3cccc4c3ccc3ccccc34)cc2)c1]

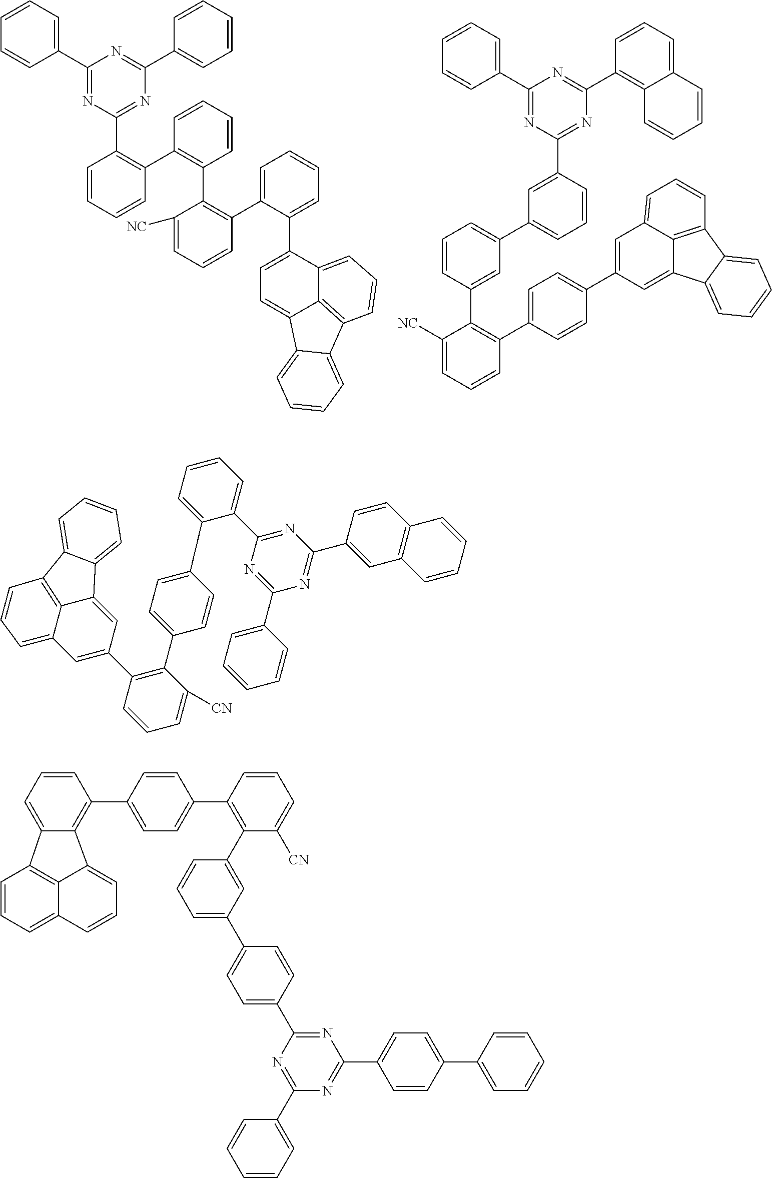

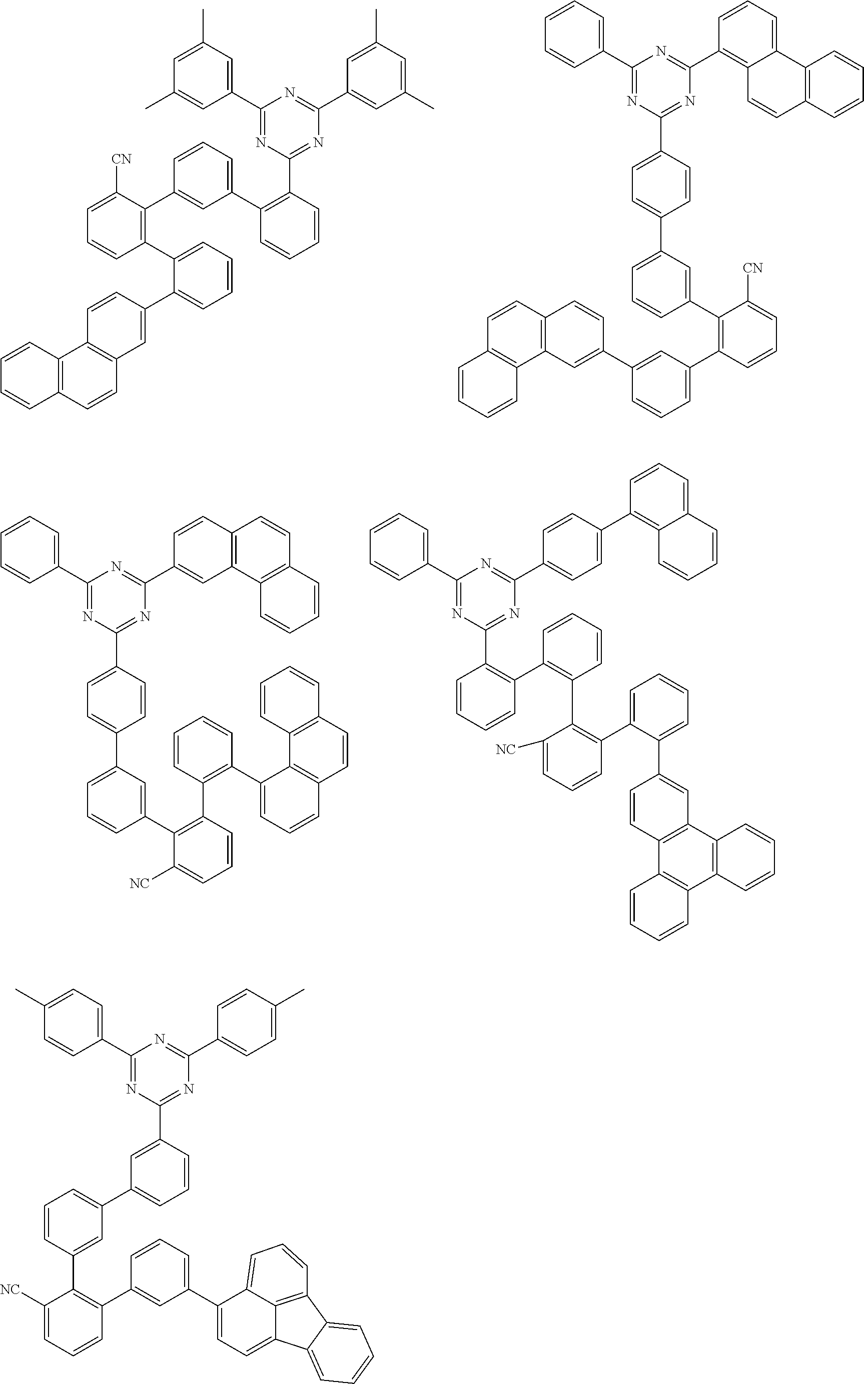

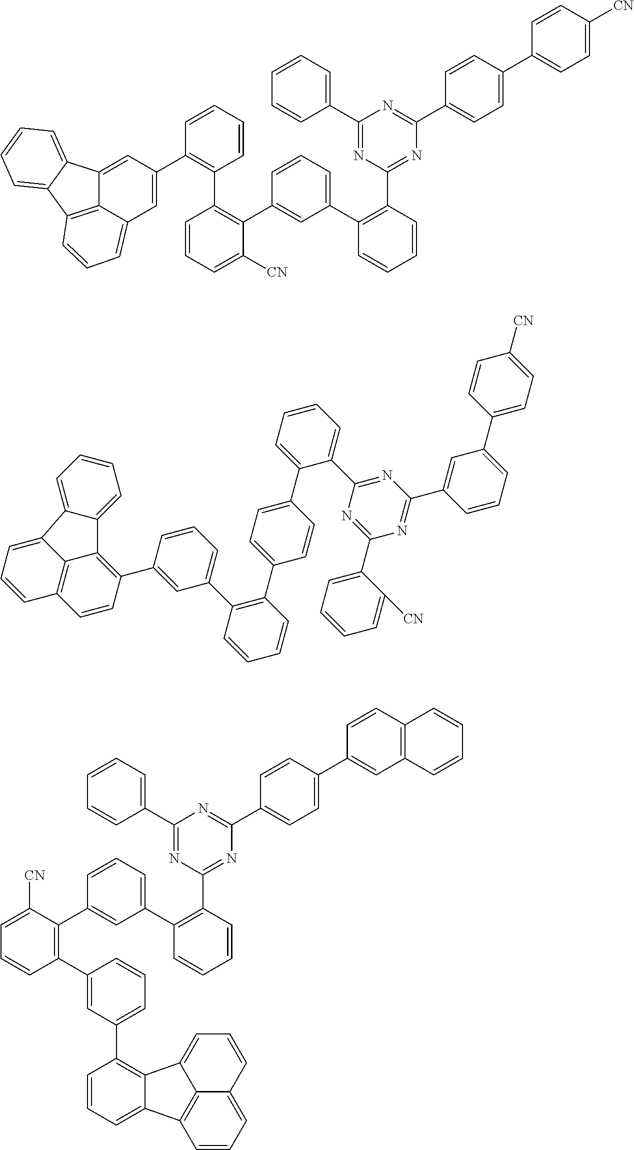 [SMILES: N#Cc1ccc(-c2ccc(-c3nc(-c4ccccc4)nc(-c4ccccc4-c4cccc(-c5c(C#N)cccc5-c5ccccc5-c5cc6c7c(cccc7c5)-c5ccccc5-6)c4)n3)cc2)cc1 ; N#Cc1ccc(-c2cccc(-c3nc(-c4ccccc4C#N)nc(-c4ccccc4-c4ccc(-c5ccccc5-c5cccc(-c6ccc7cccc8c7c6-c6ccccc6-8)c5)cc4)n3)c2)cc1 ; N#Cc1cccc(-c2cccc(-c3cccc4c3-c3cccc5cccc-4c35)c2)c1-c1cccc(-c2ccccc2-c2nc(-c3ccccc3)nc(-c3ccc(-c4ccc5ccccc5c4)cc3)n2)c1]

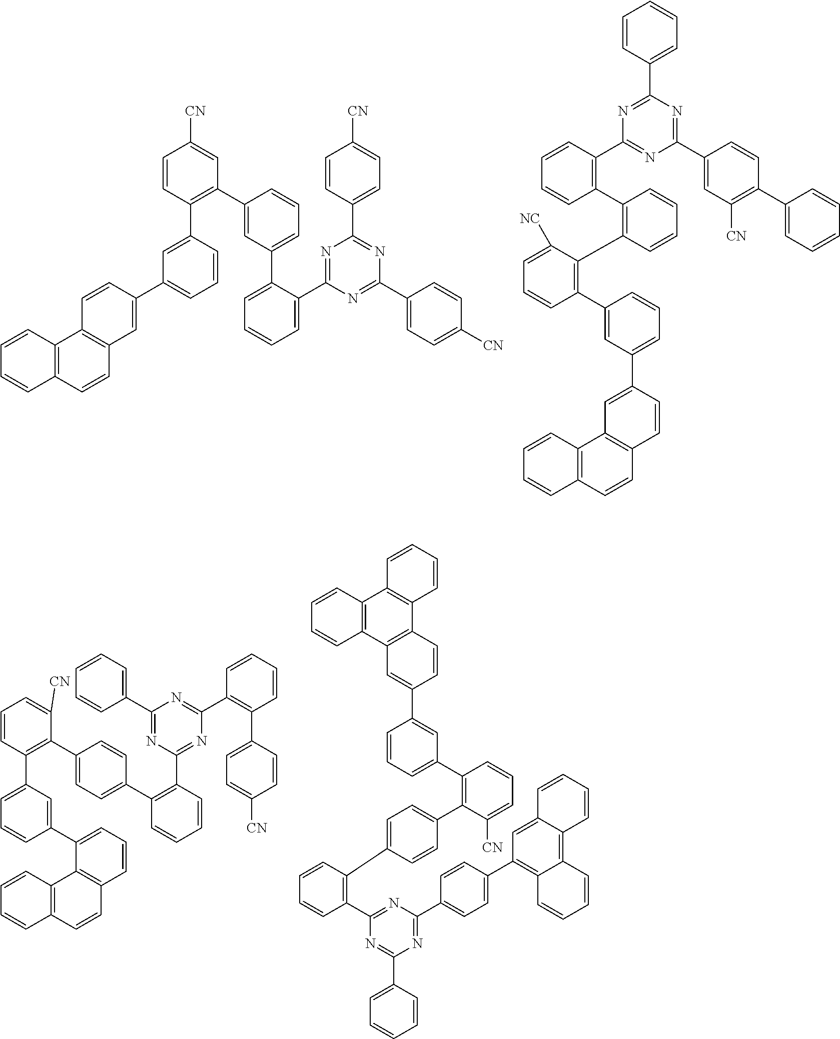 [SMILES: N#Cc1ccc(-c2nc(-c3ccc(C#N)cc3)nc(-c3ccccc3-c3cccc(-c4cc(C#N)ccc4-c4cccc(-c5ccc6c(ccc7ccccc76)c5)c4)c3)n2)cc1 ; N#Cc1cc(-c2nc(-c3ccccc3)nc(-c3ccccc3-c3ccccc3-c3c(C#N)cccc3-c3cccc(-c4ccc5ccc6ccccc6c5c4)c3)n2)ccc1-c1ccccc1 ; N#Cc1ccc(-c2ccccc2-c2nc(-c3ccccc3)nc(-c3ccccc3-c3ccc(-c4c(C#N)cccc4-c4cccc(-c5cccc6ccc7ccccc7c56)c4)cc3)n2)cc1 ; N#Cc1cccc(-c2cccc(-c3ccc4c5ccccc5c5ccccc5c4c3)c2)c1-c1ccc(-c2ccccc2-c2nc(-c3ccccc3)nc(-c3ccc(-c4cc5ccccc5c5ccccc45)cc3)n2)cc1]

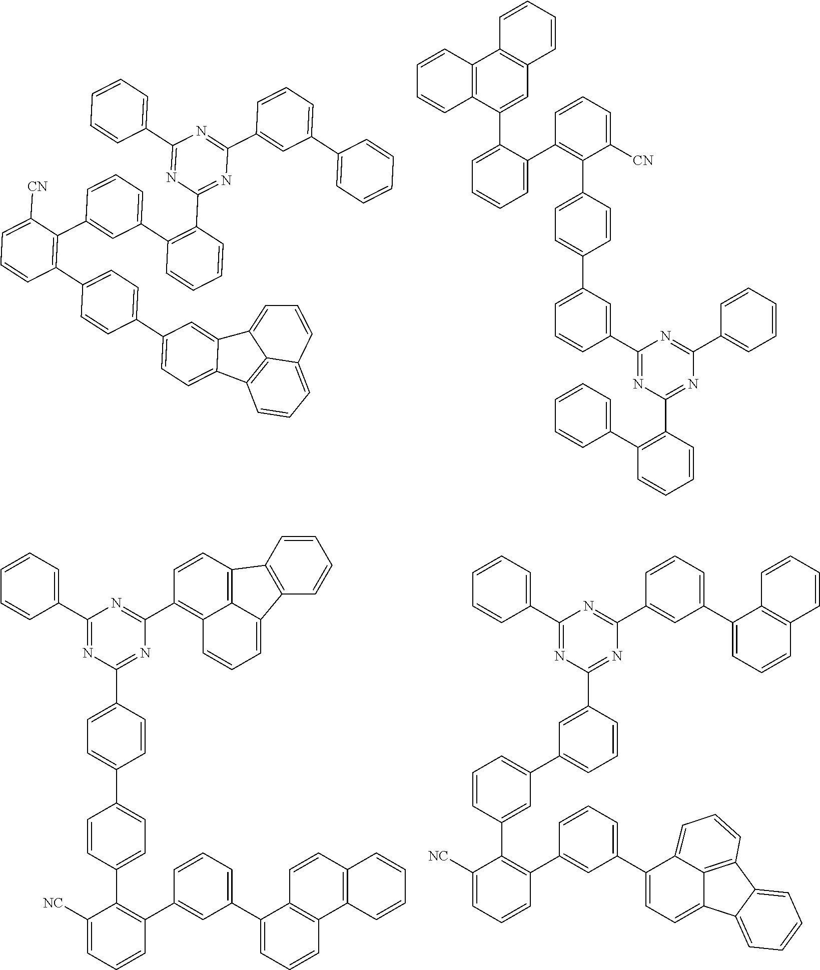

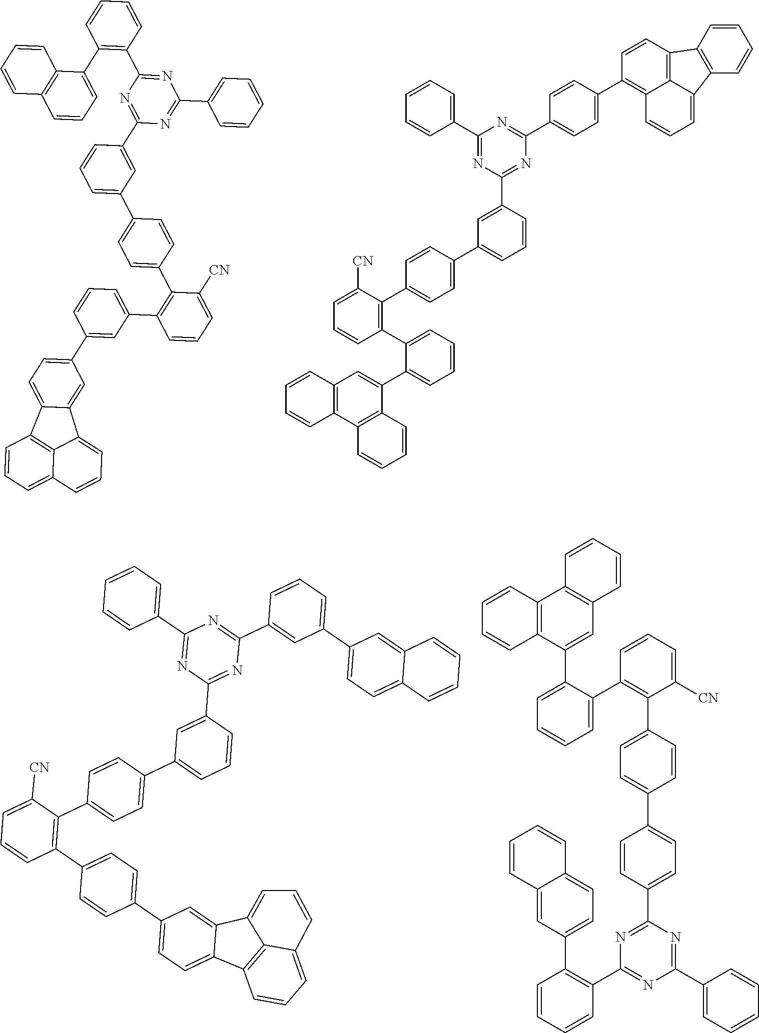

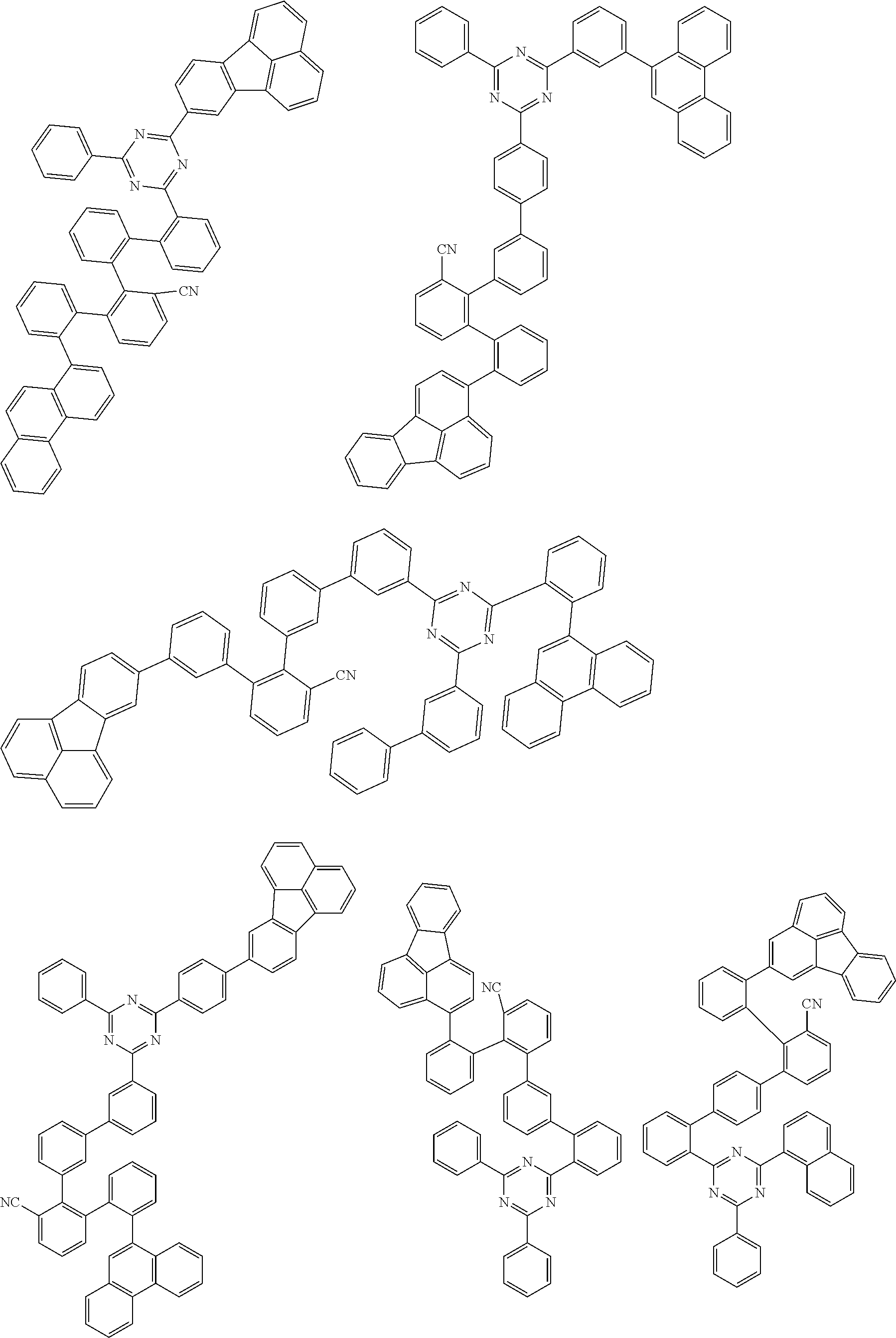

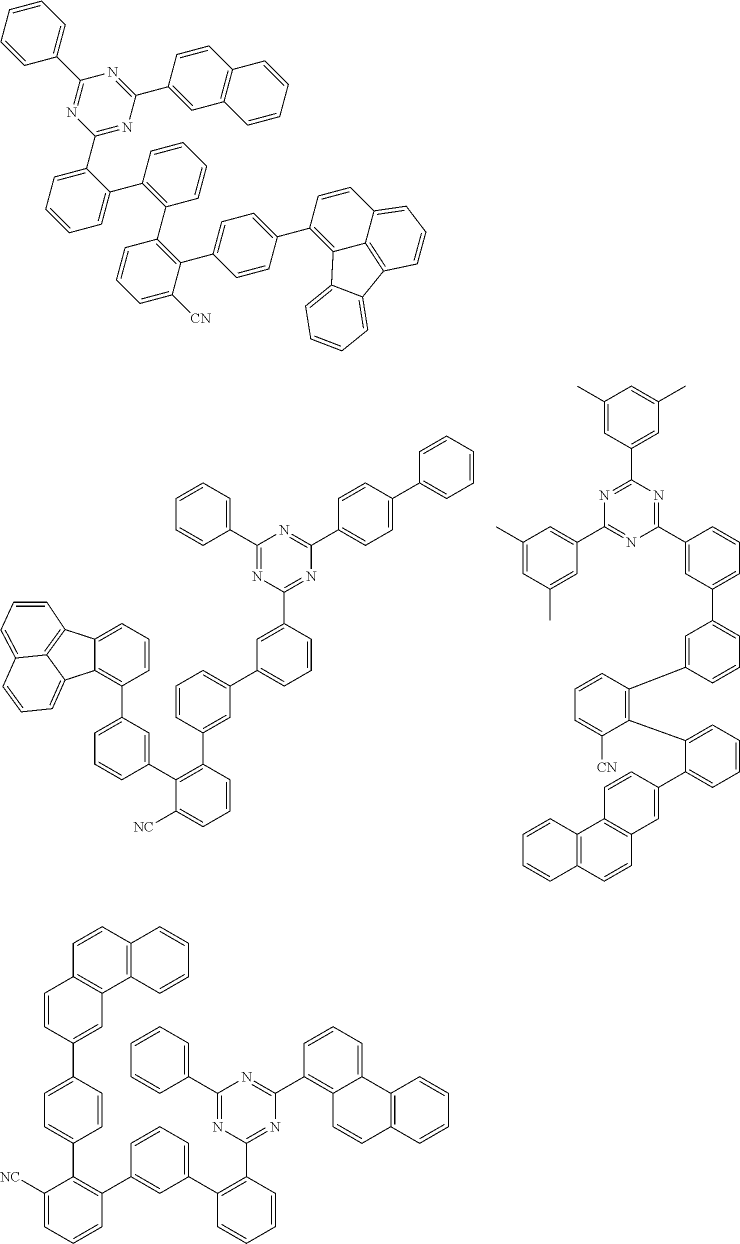 [SMILES: N#Cc1cccc(-c2ccccc2-c2ccccc2-c2nc(-c3ccccc3)nc(-c3ccc4ccccc4c3)n2)c1-c1ccc(-c2ccc3cccc4c3c2-c2ccccc2-4)cc1 ; N#Cc1cccc(-c2cccc(-c3cccc(-c4nc(-c5ccccc5)nc(-c5ccc(-c6ccccc6)cc5)n4)c3)c2)c1-c1cccc(-c2cccc3c2-c2cccc4cccc-3c24)c1 ; Cc1cc(C)cc(-c2nc(-c3cc(C)cc(C)c3)nc(-c3cccc(-c4cccc(-c5cccc(C#N)c5-c5ccccc5-c5ccc6c(ccc7ccccc76)c5)c4)c3)n2)c1 ; N#Cc1cccc(-c2cccc(-c3ccccc3-c3nc(-c4ccccc4)nc(-c4cccc5c4ccc4ccccc45)n3)c2)c1-c1ccc(-c2ccc3ccc4ccccc4c3c2)cc1]

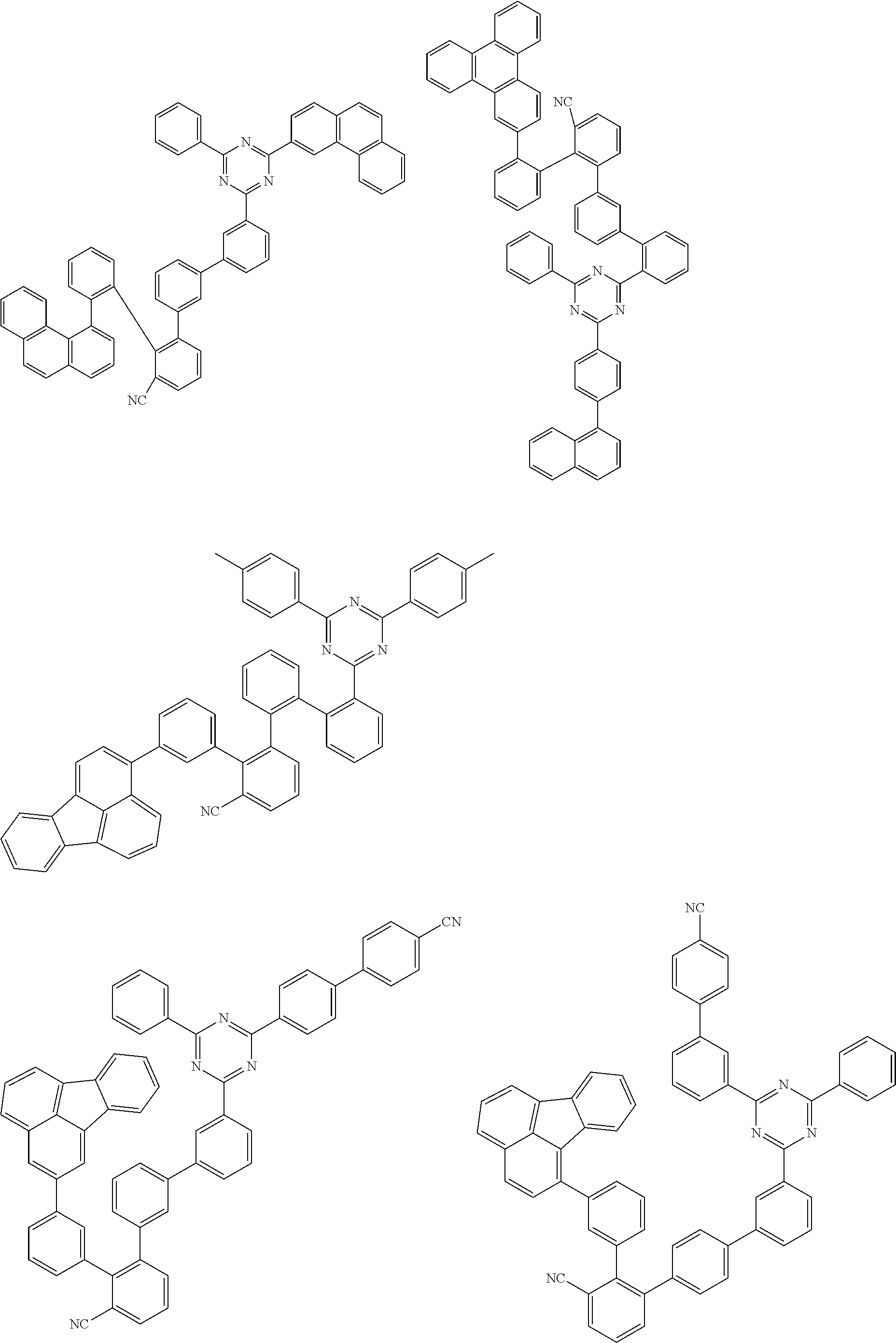

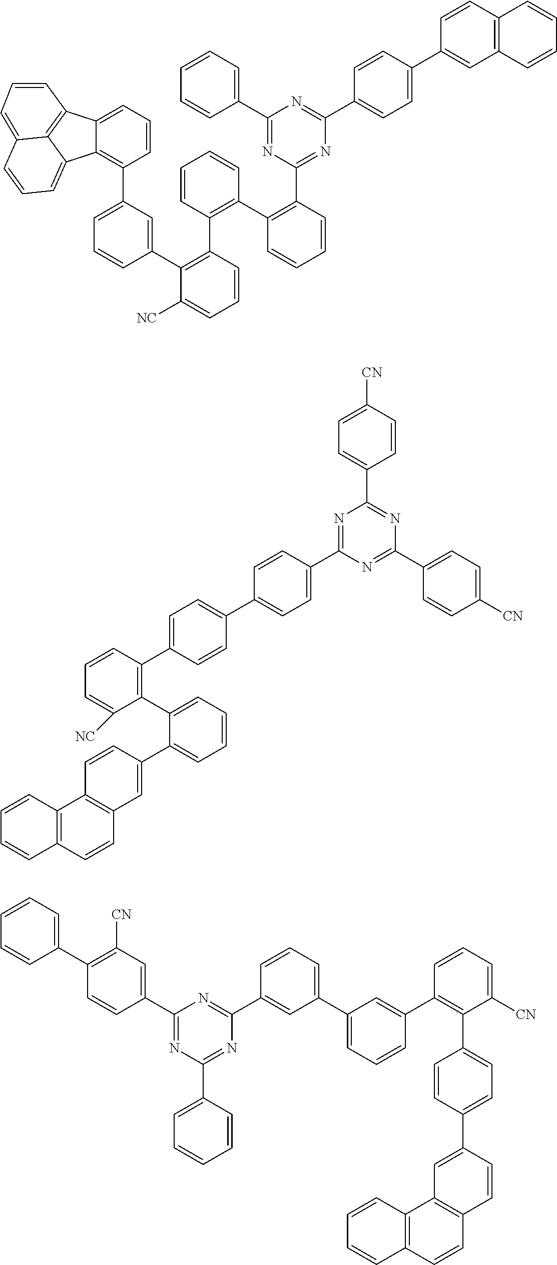

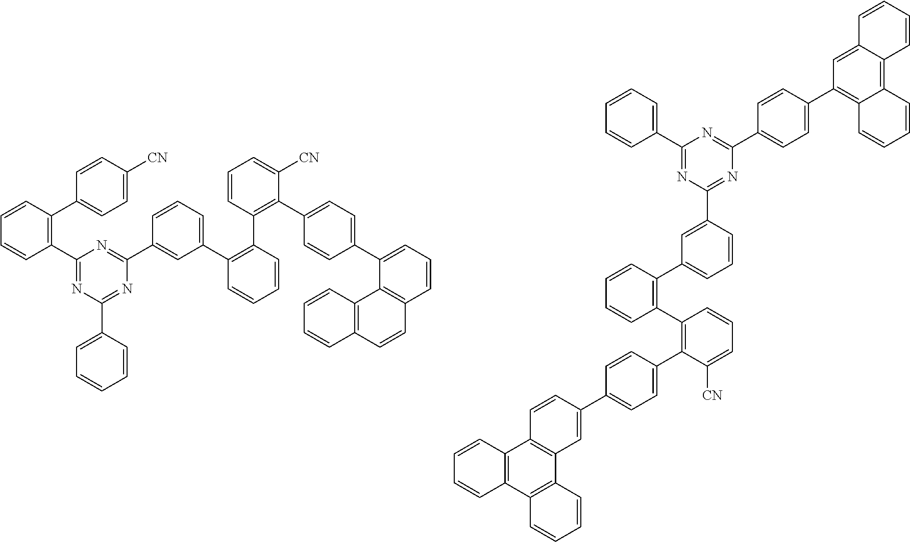 [SMILES: N#Cc1ccc(-c2ccccc2-c2nc(-c3ccccc3)nc(-c3cccc(-c4ccccc4-c4cccc(C#N)c4-c4ccc(-c5cccc6ccc7ccccc7c56)cc4)c3)n2)cc1 ; N#Cc1cccc(-c2ccccc2-c2cccc(-c3nc(-c4ccccc4)nc(-c4ccc(-c5cc6ccccc6c6ccccc56)cc4)n3)c2)c1-c1ccc(-c2ccc3c4ccccc4c4ccccc4c3c2)cc1]

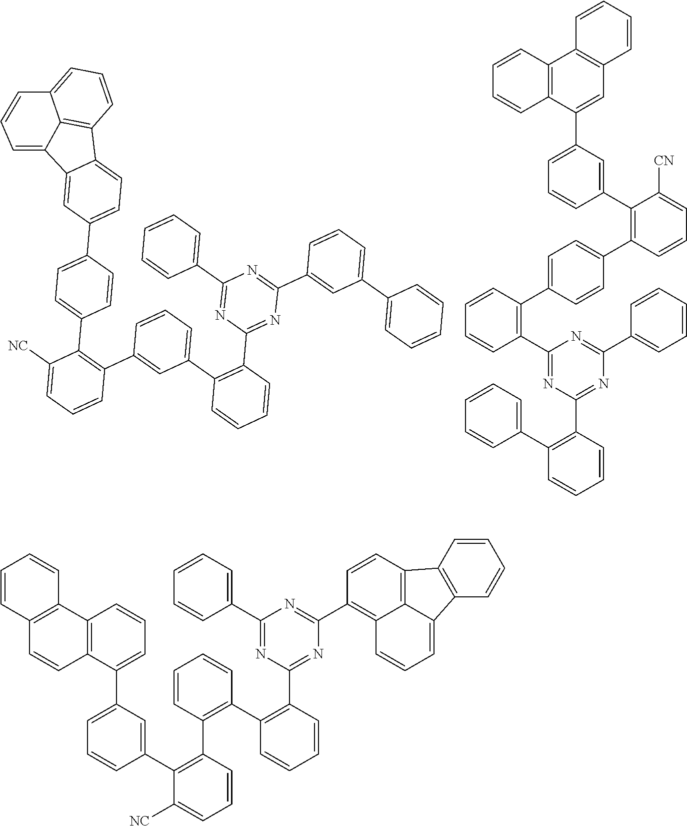

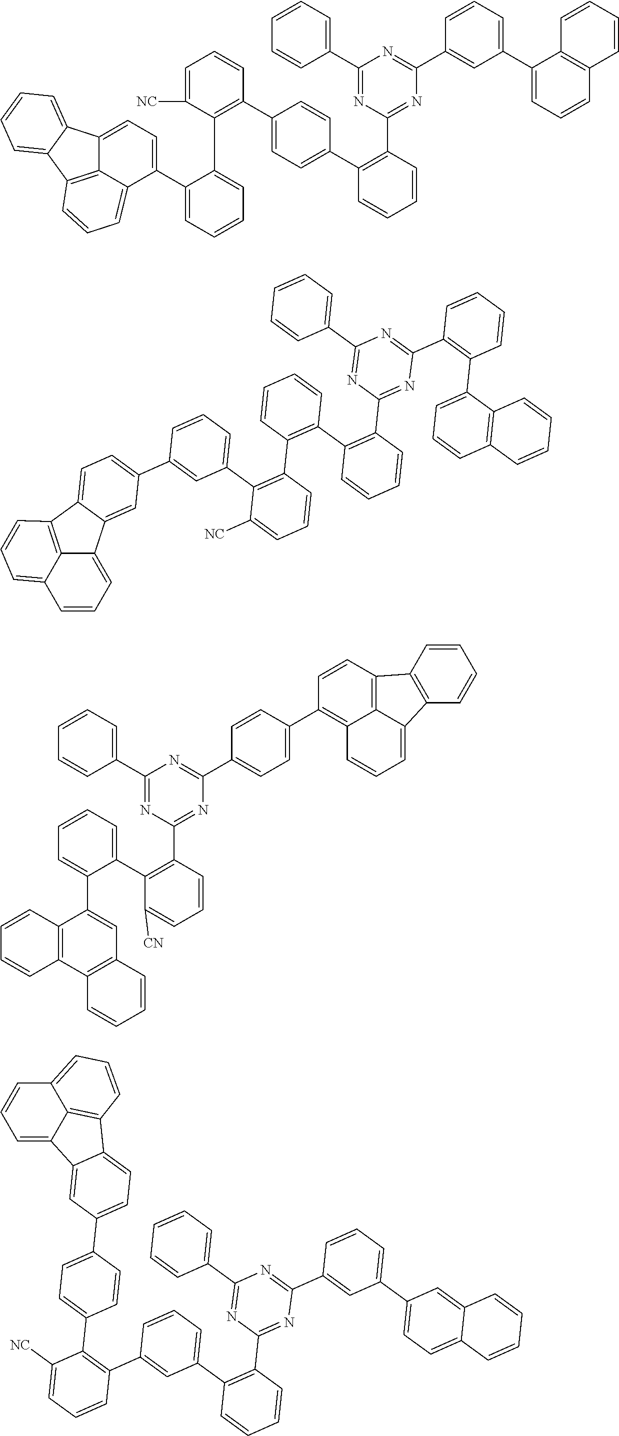

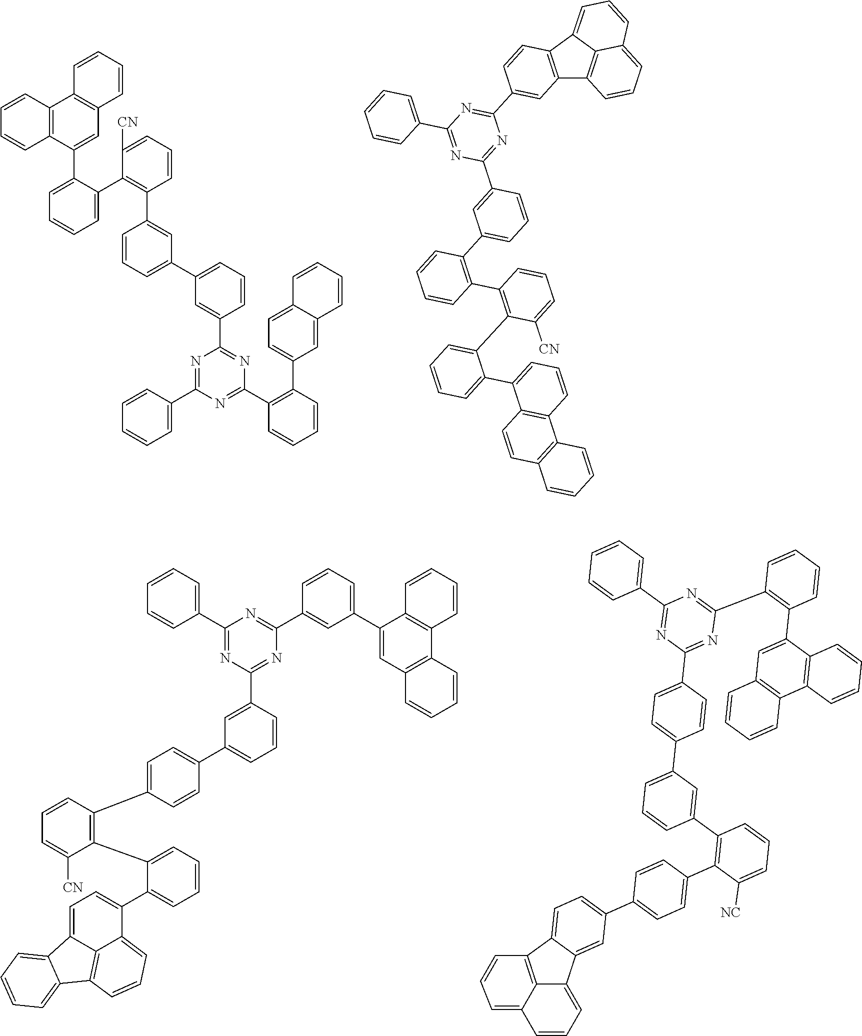 [SMILES: N#Cc1cccc(-c2cccc(-c3cccc(-c4nc(-c5ccccc5)nc(-c5ccccc5-c5ccc6ccccc6c5)n4)c3)c2)c1-c1ccccc1-c1cc2ccccc2c2ccccc12 ; N#Cc1cccc(-c2ccccc2-c2cccc(-c3nc(-c4ccccc4)nc(-c4ccc5c(c4)-c4cccc6cccc-5c46)n3)c2)c1-c1ccccc1-c1cccc2c1ccc1ccccc12 ; N#Cc1cccc(-c2ccc(-c3cccc(-c4nc(-c5ccccc5)nc(-c5cccc(-c6cc7ccccc7c7ccccc67)c5)n4)c3)cc2)c1-c1ccccc1-c1ccc2c3c(cccc13)-c1ccccc1-2 ; N#Cc1cccc(-c2cccc(-c3ccc(-c4nc(-c5ccccc5)nc(-c5ccccc5-c5cc6ccccc6c6ccccc56)n4)cc3)c2)c1-c1ccc(-c2ccc3c(c2)-c2cccc4cccc-3c24)cc1]

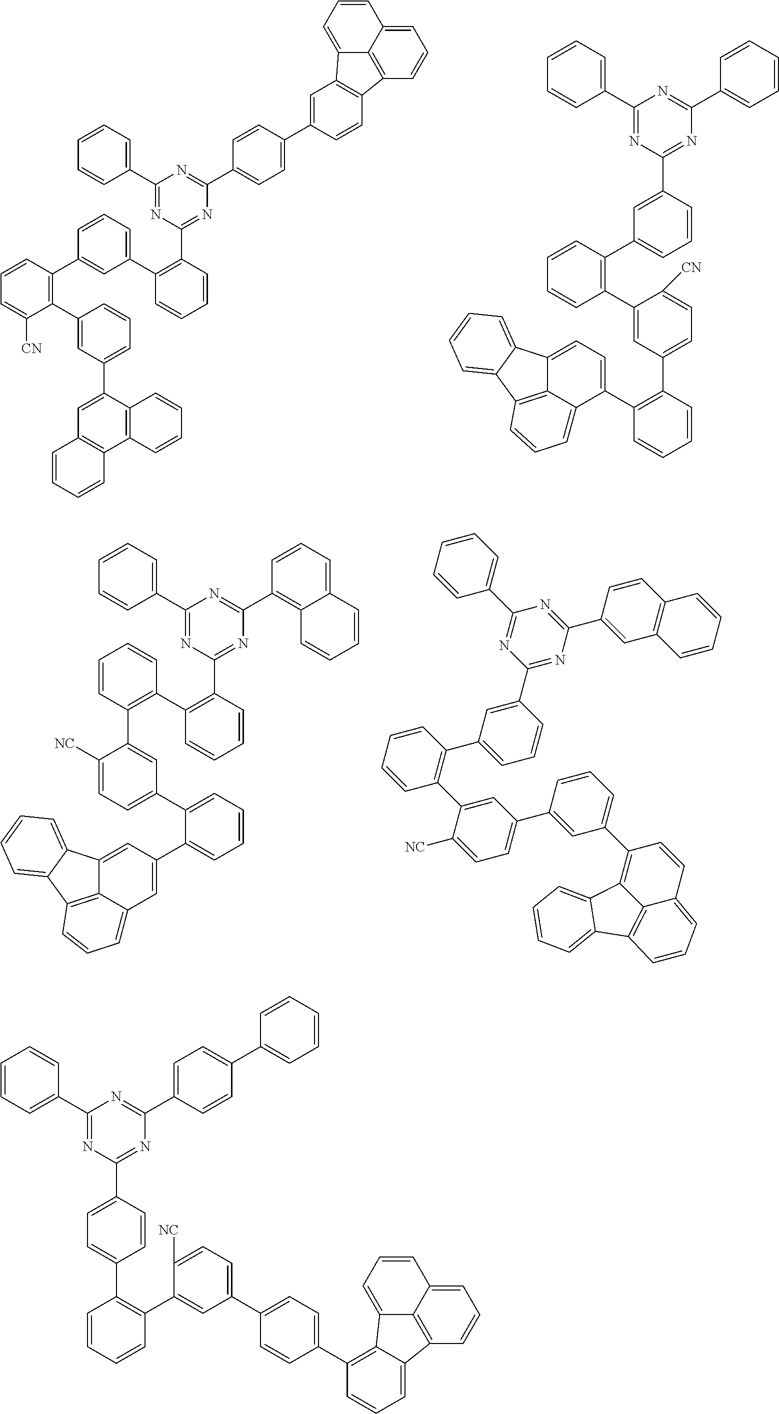

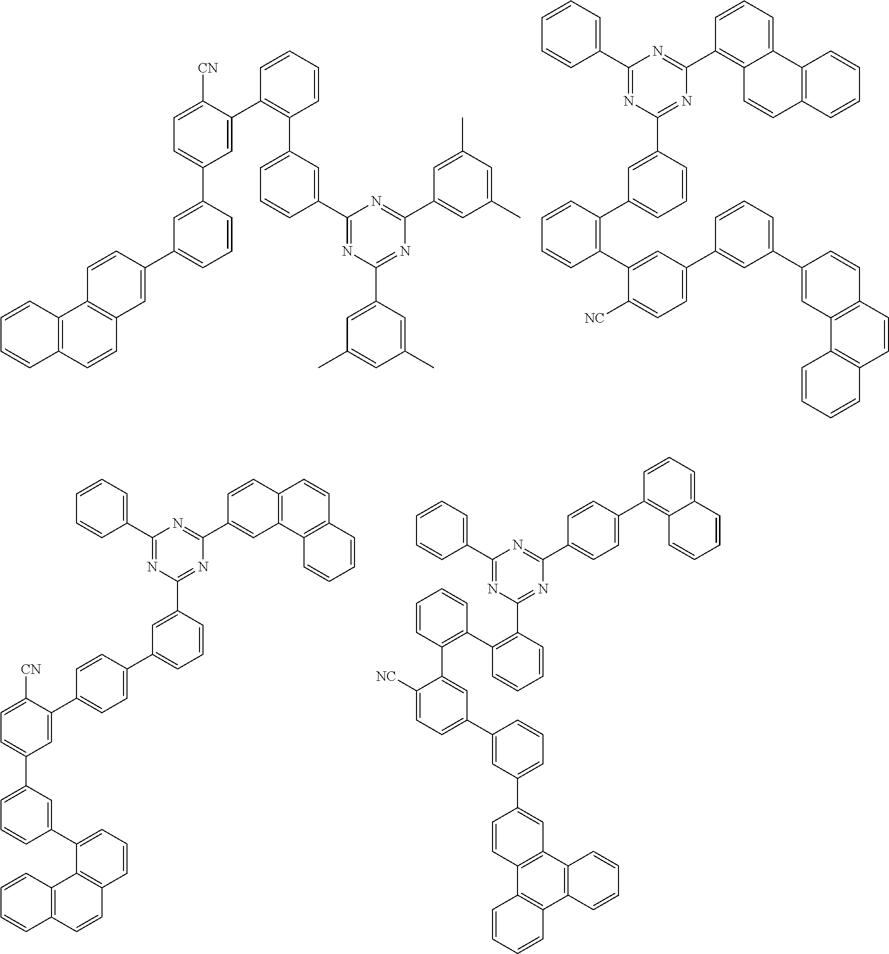

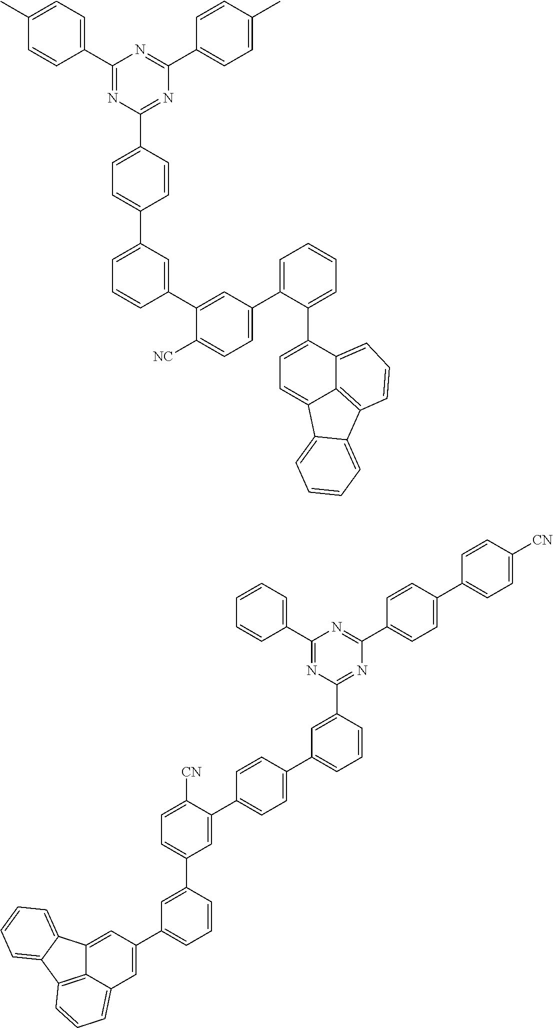 [SMILES: Cc1ccc(-c2nc(-c3ccc(C)cc3)nc(-c3ccc(-c4cccc(-c5cc(-c6ccccc6-c6ccc7c8c(cccc68)-c6ccccc6-7)ccc5C#N)c4)cc3)n2)cc1 ; N#Cc1ccc(-c2ccc(-c3nc(-c4ccccc4)nc(-c4cccc(-c5ccc(-c6cc(-c7cccc(-c8cc9c%10c(cccc%10c8)-c8ccccc8-9)c7)ccc6C#N)cc5)c4)n3)cc2)cc1]

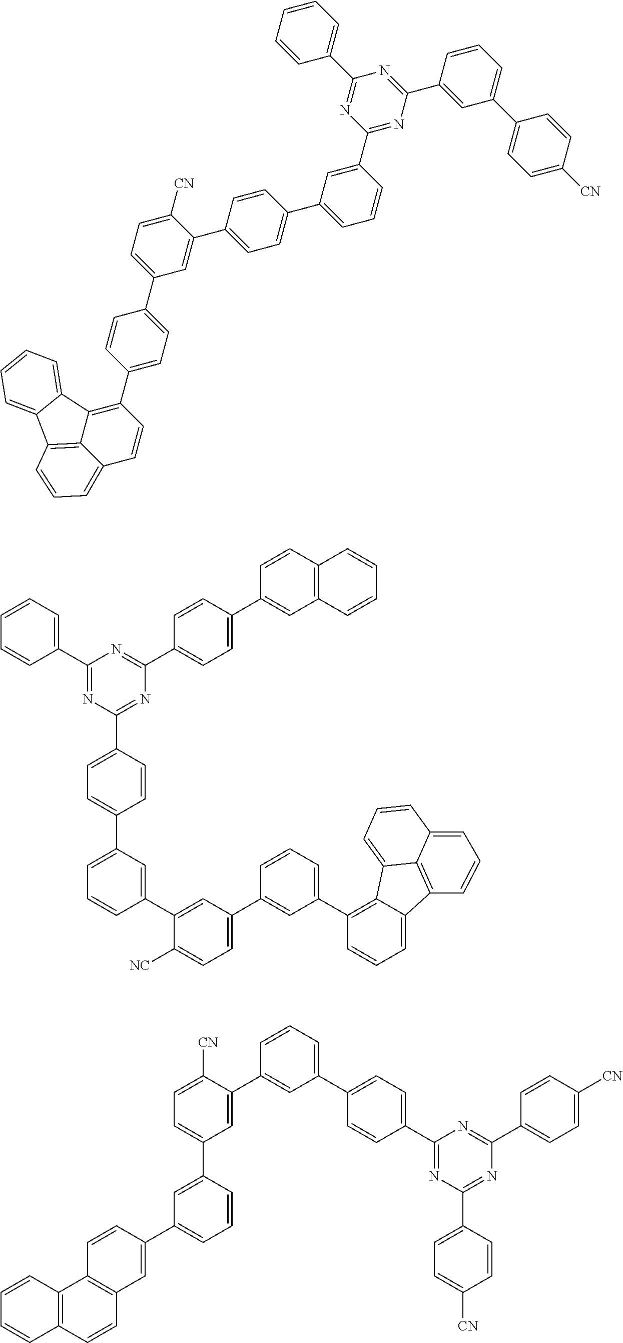 [SMILES: N#Cc1ccc(-c2cccc(-c3nc(-c4ccccc4)nc(-c4cccc(-c5ccc(-c6cc(-c7ccc(-c8ccc9cccc%10c9c8-c8ccccc8-%10)cc7)ccc6C#N)cc5)c4)n3)c2)cc1 ; N#Cc1ccc(-c2cccc(-c3cccc4c3-c3cccc5cccc-4c35)c2)cc1-c1cccc(-c2ccc(-c3nc(-c4ccccc4)nc(-c4ccc(-c5ccc6ccccc6c5)cc4)n3)cc2)c1 ; N#Cc1ccc(-c2nc(-c3ccc(C#N)cc3)nc(-c3ccc(-c4cccc(-c5cc(-c6cccc(-c7ccc8c(ccc9ccccc98)c7)c6)ccc5C#N)c4)cc3)n2)cc1]

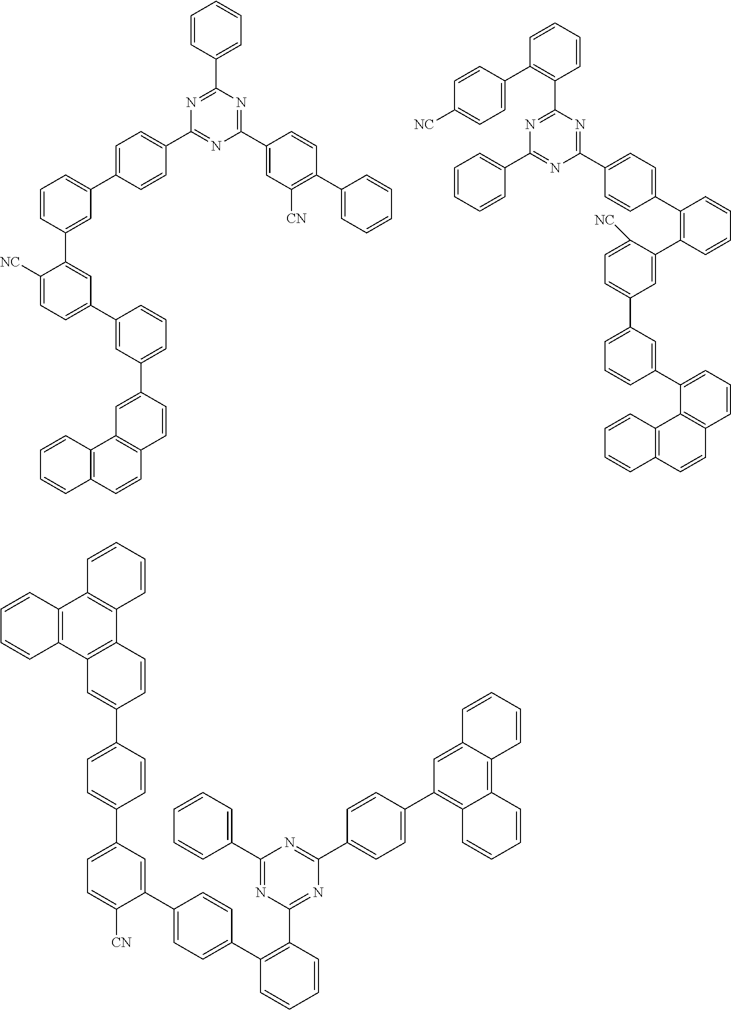 [SMILES: N#Cc1cc(-c2nc(-c3ccccc3)nc(-c3ccc(-c4cccc(-c5cc(-c6cccc(-c7ccc8ccc9ccccc9c8c7)c6)ccc5C#N)c4)cc3)n2)ccc1-c1ccccc1 ; N#Cc1ccc(-c2ccccc2-c2nc(-c3ccccc3)nc(-c3ccc(-c4ccccc4-c4cc(-c5cccc(-c6cccc7ccc8ccccc8c67)c5)ccc4C#N)cc3)n2)cc1 ; N#Cc1ccc(-c2ccc(-c3ccc4c5ccccc5c5ccccc5c4c3)cc2)cc1-c1ccc(-c2ccccc2-c2nc(-c3ccccc3)nc(-c3ccc(-c4cc5ccccc5c5ccccc45)cc3)n2)cc1]

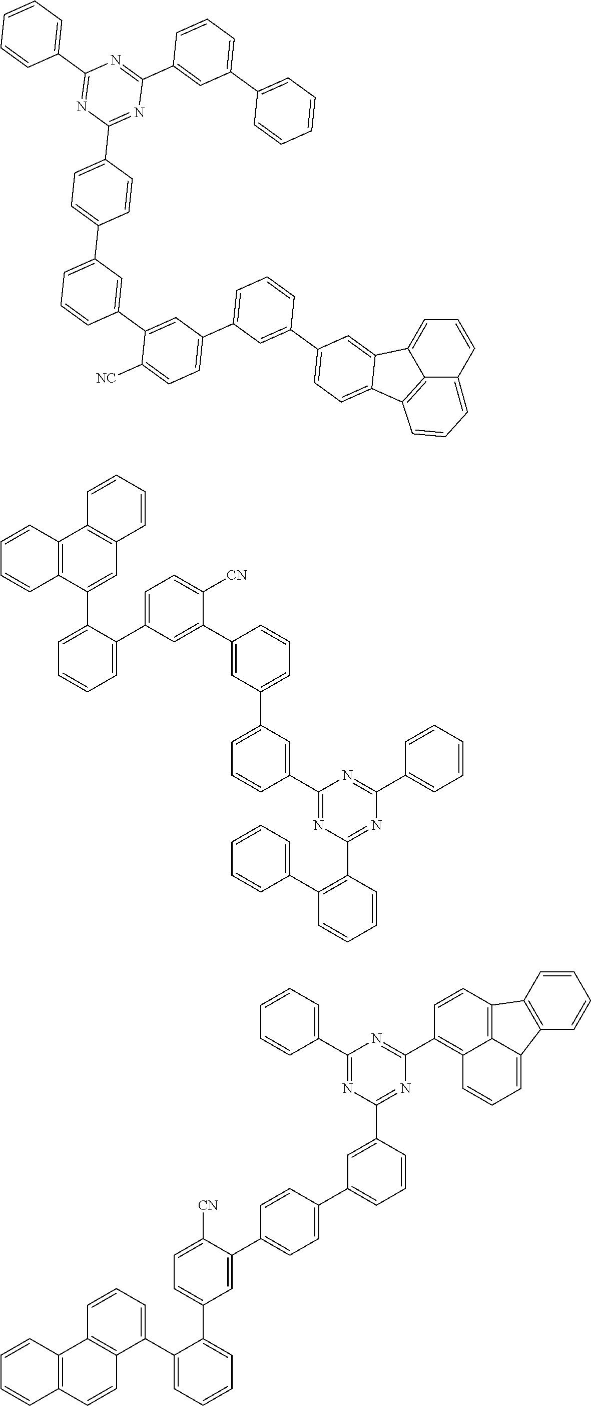 [SMILES: N#Cc1ccc(-c2cccc(-c3ccc4c(c3)-c3cccc5cccc-4c35)c2)cc1-c1cccc(-c2ccc(-c3nc(-c4ccccc4)nc(-c4cccc(-c5ccccc5)c4)n3)cc2)c1 ; N#Cc1ccc(-c2ccccc2-c2cc3ccccc3c3ccccc23)cc1-c1cccc(-c2cccc(-c3nc(-c4ccccc4)nc(-c4ccccc4-c4ccccc4)n3)c2)c1 ; N#Cc1ccc(-c2ccccc2-c2cccc3c2ccc2ccccc23)cc1-c1ccc(-c2cccc(-c3nc(-c4ccccc4)nc(-c4ccc5c6c(cccc46)-c4ccccc4-5)n3)c2)cc1]

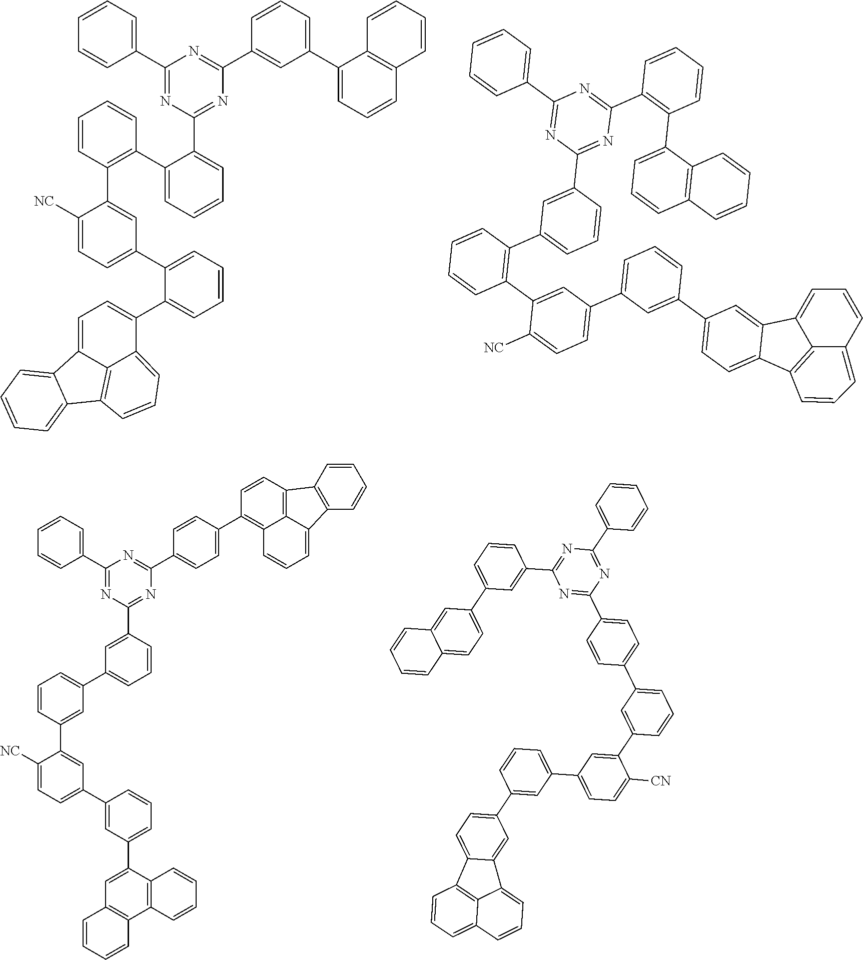 [SMILES: N#Cc1ccc(-c2ccccc2-c2ccc3c4c(cccc24)-c2ccccc2-3)cc1-c1ccccc1-c1ccccc1-c1nc(-c2ccccc2)nc(-c2cccc(-c3cccc4ccccc34)c2)n1 ; N#Cc1ccc(-c2cccc(-c3ccc4c(c3)-c3cccc5cccc-4c35)c2)cc1-c1ccccc1-c1cccc(-c2nc(-c3ccccc3)nc(-c3ccccc3-c3cccc4ccccc34)n2)c1 ; N#Cc1ccc(-c2cccc(-c3cc4ccccc4c4ccccc34)c2)cc1-c1cccc(-c2cccc(-c3nc(-c4ccccc4)nc(-c4ccc(-c5ccc6c7c(cccc57)-c5ccccc5-6)cc4)n3)c2)c1 ; N#Cc1ccc(-c2cccc(-c3ccc4c(c3)-c3cccc5cccc-4c35)c2)cc1-c1cccc(-c2ccc(-c3nc(-c4ccccc4)nc(-c4cccc(-c5ccc6ccccc6c5)c4)n3)cc2)c1]

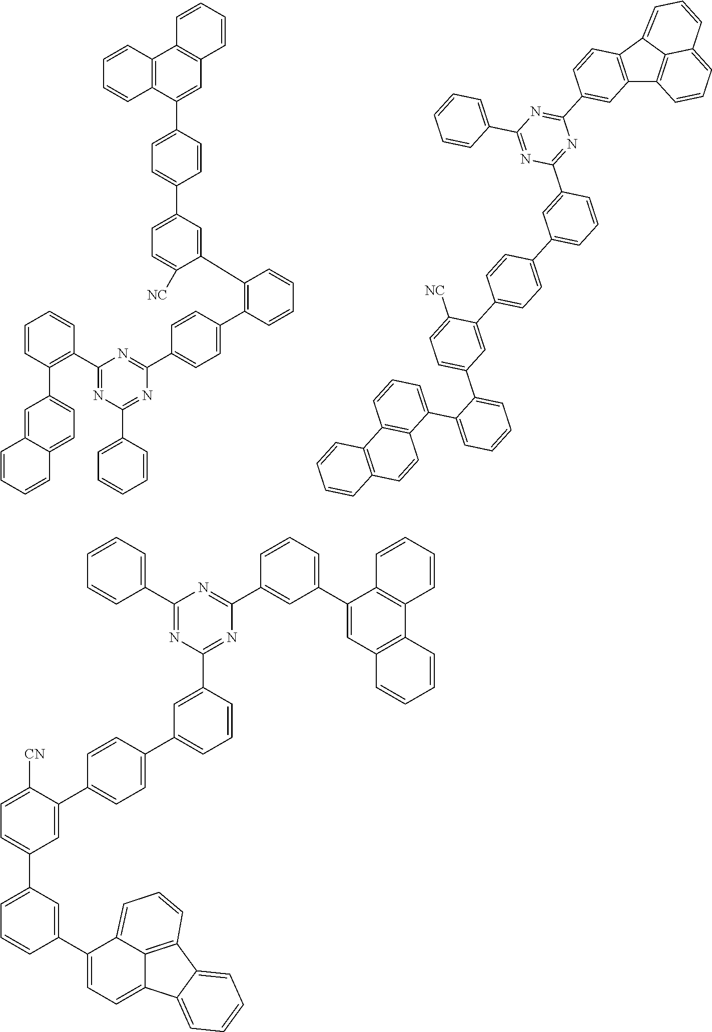 [SMILES: N#Cc1ccc(-c2ccc(-c3cc4ccccc4c4ccccc34)cc2)cc1-c1ccccc1-c1ccc(-c2nc(-c3ccccc3)nc(-c3ccccc3-c3ccc4ccccc4c3)n2)cc1 ; N#Cc1ccc(-c2ccccc2-c2cccc3c2ccc2ccccc23)cc1-c1ccc(-c2cccc(-c3nc(-c4ccccc4)nc(-c4ccc5c(c4)-c4cccc6cccc-5c46)n3)c2)cc1 ; N#Cc1ccc(-c2cccc(-c3ccc4c5c(cccc35)-c3ccccc3-4)c2)cc1-c1ccc(-c2cccc(-c3nc(-c4ccccc4)nc(-c4cccc(-c5cc6ccccc6c6ccccc56)c4)n3)c2)cc1]

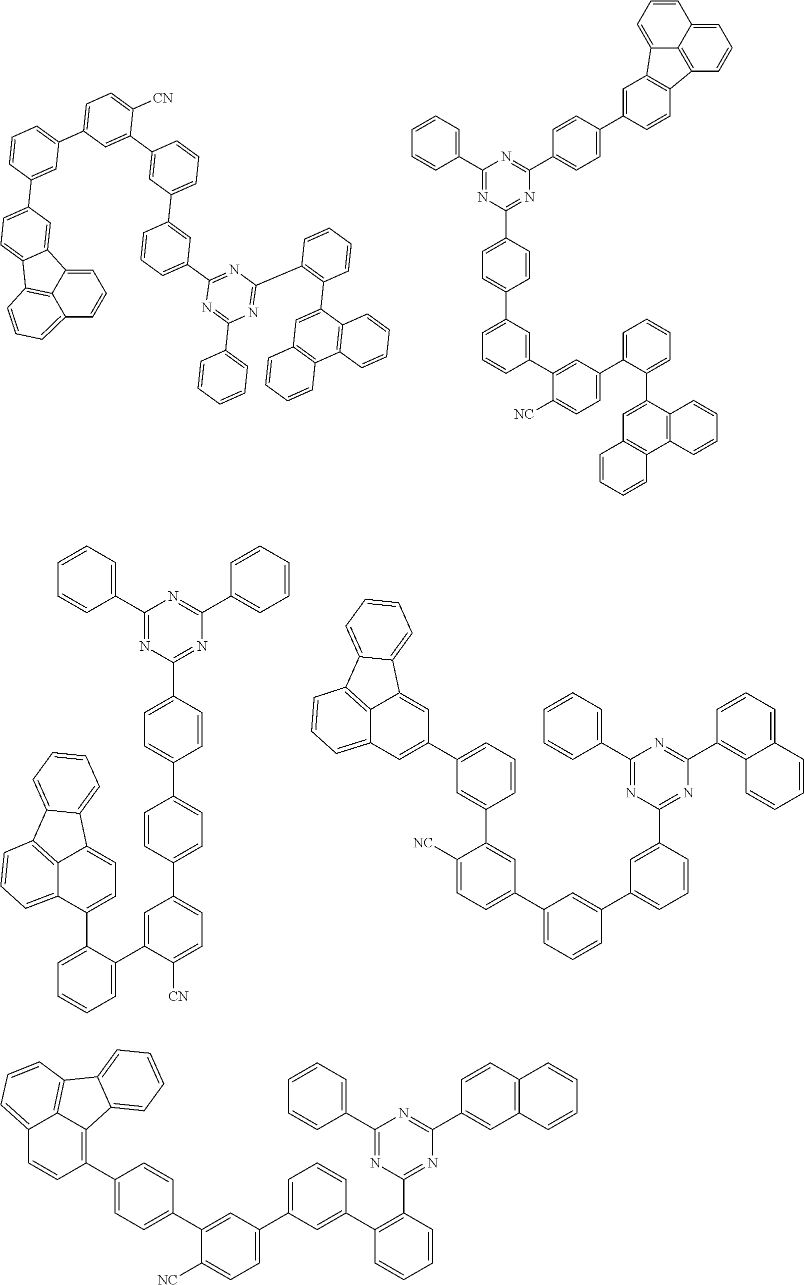

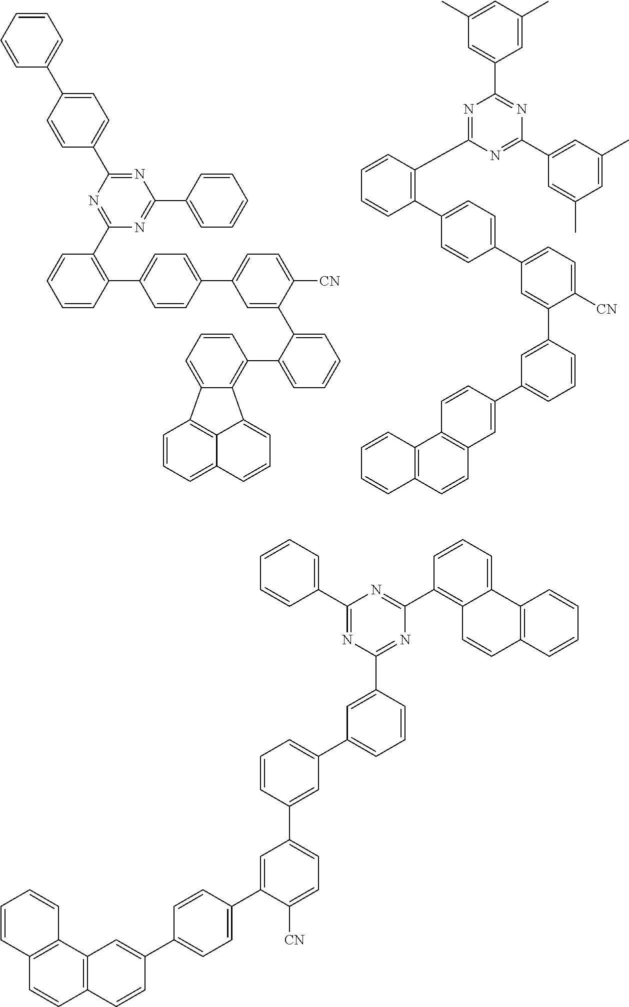 [SMILES: N#Cc1ccc(-c2ccc(-c3ccccc3-c3nc(-c4ccccc4)nc(-c4ccc(-c5ccccc5)cc4)n3)cc2)cc1-c1ccccc1-c1cccc2c1-c1cccc3cccc-2c13 ; Cc1cc(C)cc(-c2nc(-c3cc(C)cc(C)c3)nc(-c3ccccc3-c3ccc(-c4ccc(C#N)c(-c5cccc(-c6ccc7c(ccc8ccccc87)c6)c5)c4)cc3)n2)c1 ; N#Cc1ccc(-c2cccc(-c3cccc(-c4nc(-c5ccccc5)nc(-c5cccc6c5ccc5ccccc56)n4)c3)c2)cc1-c1ccc(-c2ccc3ccc4ccccc4c3c2)cc1]

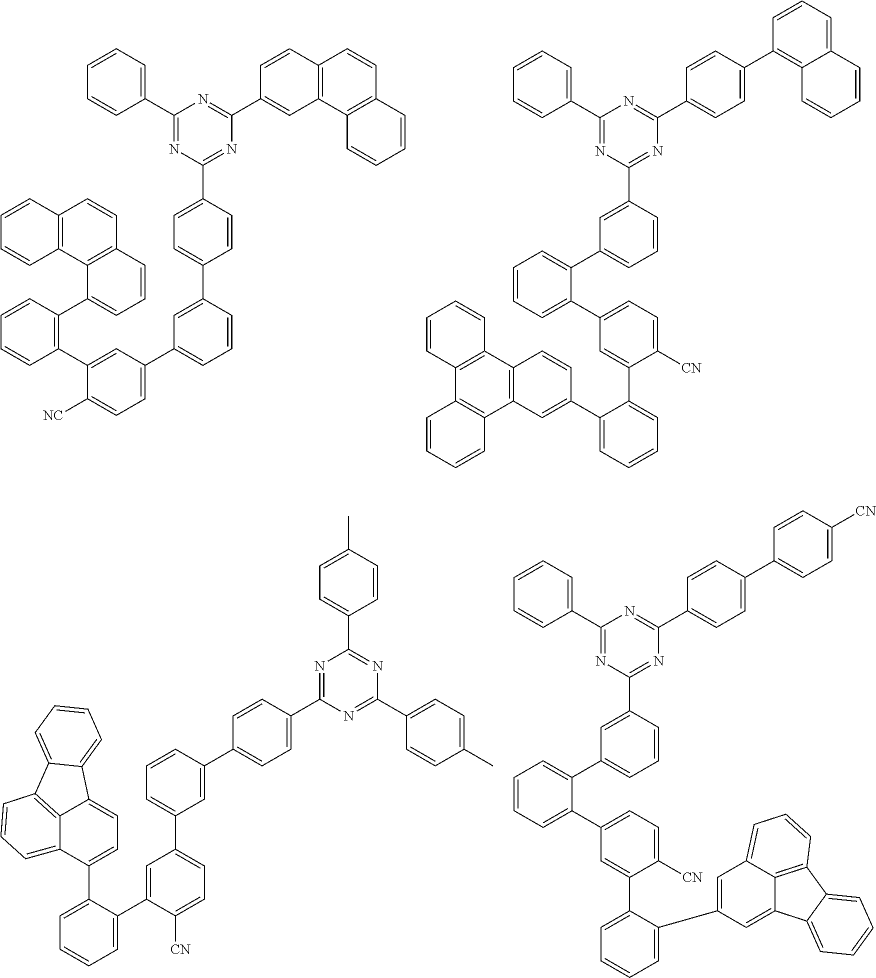

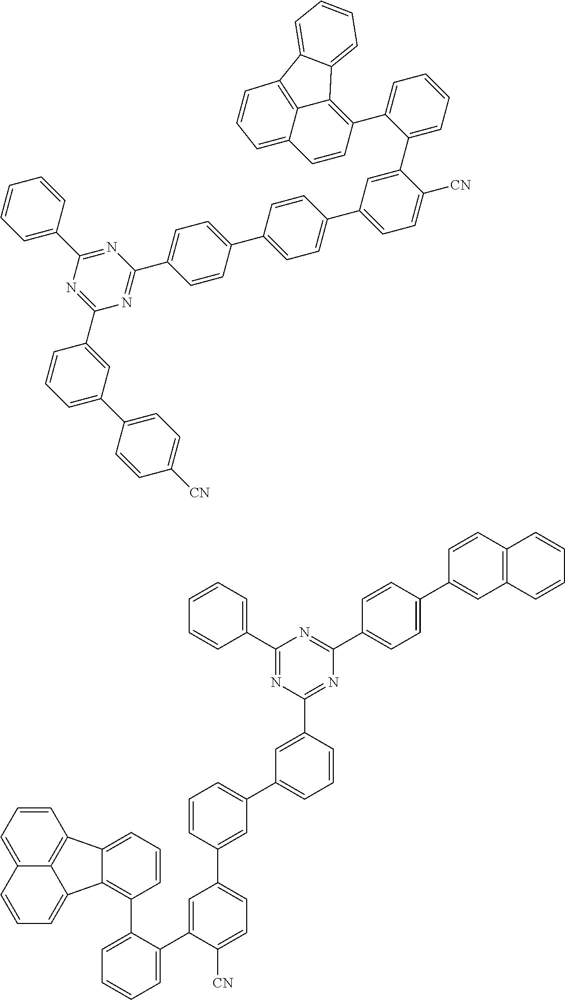 [SMILES: N#Cc1ccc(-c2cccc(-c3nc(-c4ccccc4)nc(-c4ccc(-c5ccc(-c6ccc(C#N)c(-c7ccccc7-c7ccc8cccc9c8c7-c7ccccc7-9)c6)cc5)cc4)n3)c2)cc1 ; N#Cc1ccc(-c2cccc(-c3cccc(-c4nc(-c5ccccc5)nc(-c5ccc(-c6ccc7ccccc7c6)cc5)n4)c3)c2)cc1-c1ccccc1-c1cccc2c1-c1cccc3cccc-2c13]

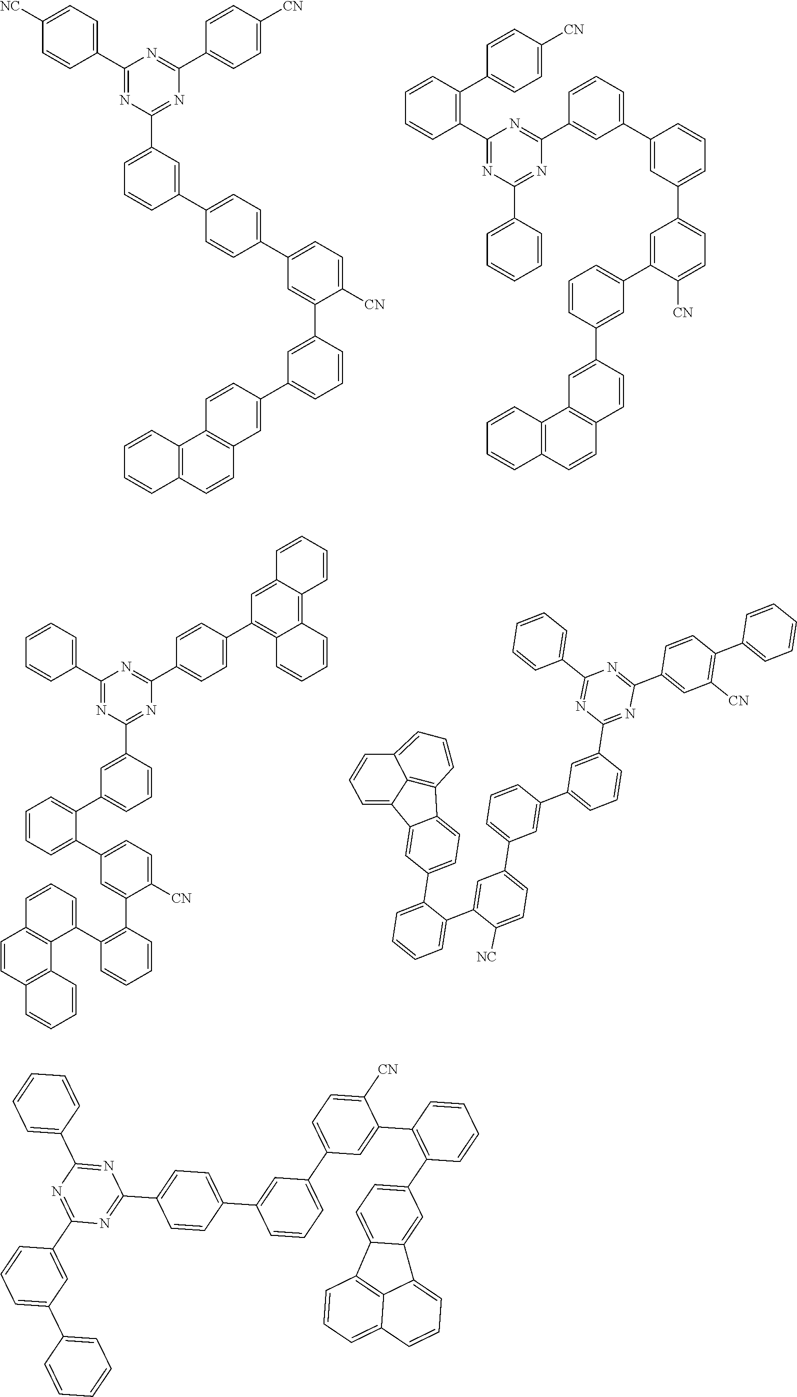

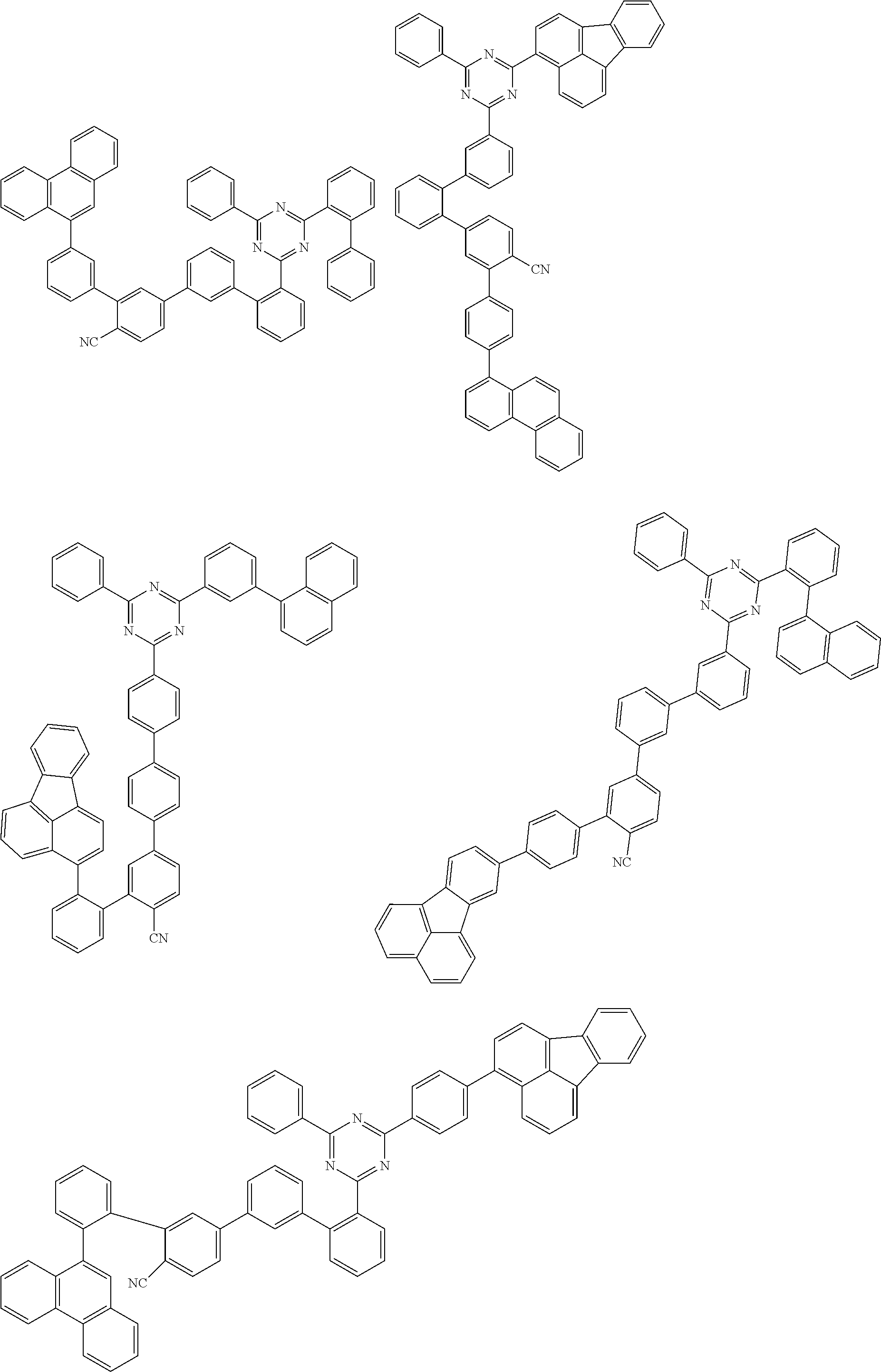

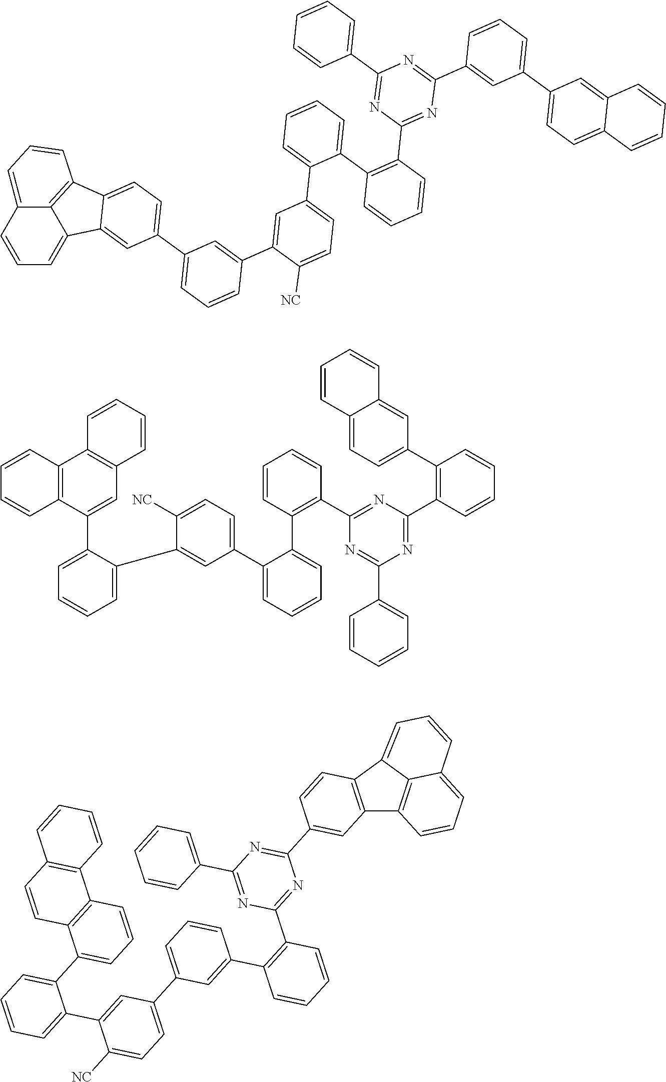 [SMILES: N#Cc1ccc(-c2ccccc2-c2ccccc2-c2nc(-c3ccccc3)nc(-c3cccc(-c4ccc5ccccc5c4)c3)n2)cc1-c1cccc(-c2ccc3c(c2)-c2cccc4cccc-3c24)c1 ; N#Cc1ccc(-c2ccccc2-c2ccccc2-c2nc(-c3ccccc3)nc(-c3ccccc3-c3ccc4ccccc4c3)n2)cc1-c1ccccc1-c1cc2ccccc2c2ccccc12 ; N#Cc1ccc(-c2cccc(-c3ccccc3-c3nc(-c4ccccc4)nc(-c4ccc5c(c4)-c4cccc6cccc-5c46)n3)c2)cc1-c1ccccc1-c1cccc2c1ccc1ccccc12]

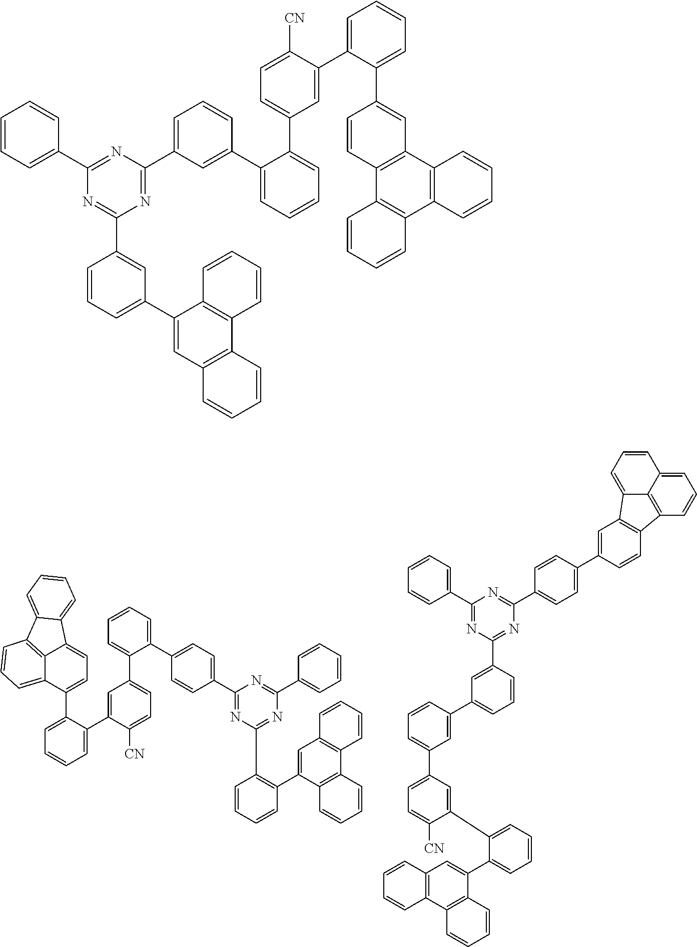 [SMILES: N#Cc1ccc(-c2ccccc2-c2cccc(-c3nc(-c4ccccc4)nc(-c4cccc(-c5cc6ccccc6c6ccccc56)c4)n3)c2)cc1-c1ccccc1-c1ccc2c3ccccc3c3ccccc3c2c1 ; N#Cc1ccc(-c2ccccc2-c2ccc(-c3nc(-c4ccccc4)nc(-c4ccccc4-c4cc5ccccc5c5ccccc45)n3)cc2)cc1-c1ccccc1-c1ccc2c3c(cccc13)-c1ccccc1-2 ; N#Cc1ccc(-c2cccc(-c3cccc(-c4nc(-c5ccccc5)nc(-c5ccc(-c6ccc7c(c6)-c6cccc8cccc-7c68)cc5)n4)c3)c2)cc1-c1ccccc1-c1cc2ccccc2c2ccccc12]

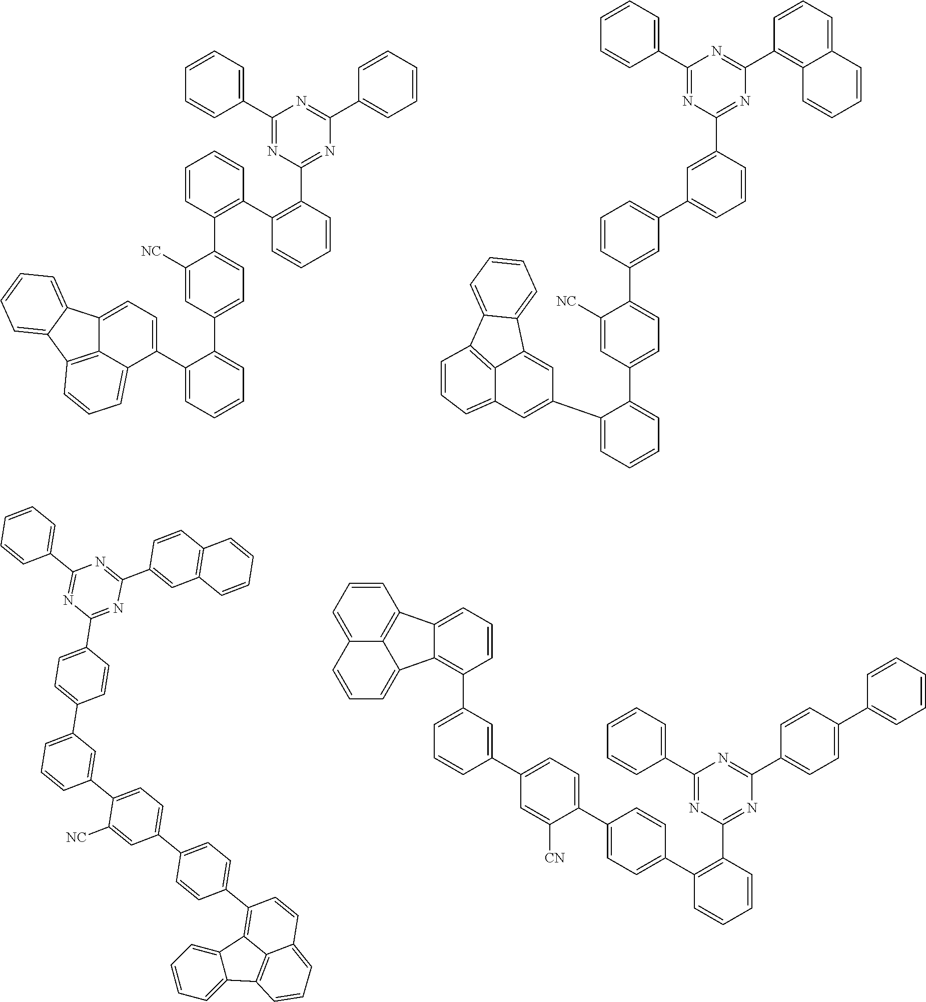 [SMILES: N#Cc1cc(-c2ccccc2-c2ccc3c4c(cccc24)-c2ccccc2-3)ccc1-c1ccccc1-c1ccccc1-c1nc(-c2ccccc2)nc(-c2ccccc2)n1 ; N#Cc1cc(-c2ccccc2-c2cc3c4c(cccc4c2)-c2ccccc2-3)ccc1-c1cccc(-c2cccc(-c3nc(-c4ccccc4)nc(-c4cccc5ccccc45)n3)c2)c1 ; N#Cc1cc(-c2ccc(-c3ccc4cccc5c4c3-c3ccccc3-5)cc2)ccc1-c1cccc(-c2ccc(-c3nc(-c4ccccc4)nc(-c4ccc5ccccc5c4)n3)cc2)c1 ; N#Cc1cc(-c2cccc(-c3cccc4c3-c3cccc5cccc-4c35)c2)ccc1-c1ccc(-c2ccccc2-c2nc(-c3ccccc3)nc(-c3ccc(-c4ccccc4)cc3)n2)cc1]

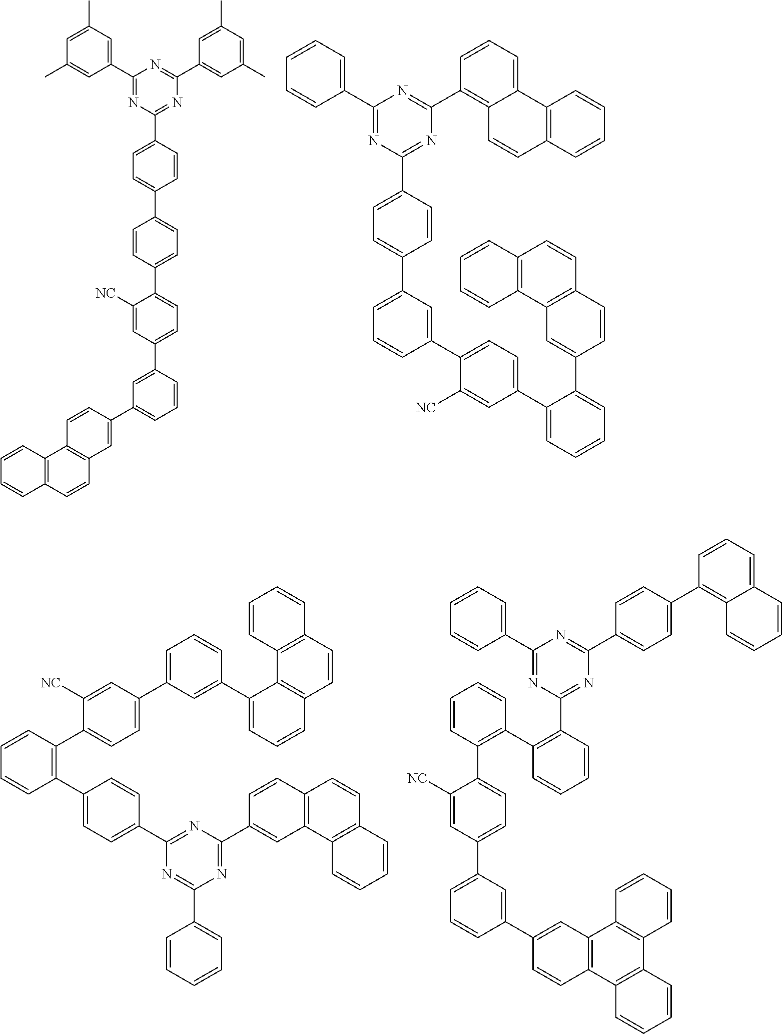

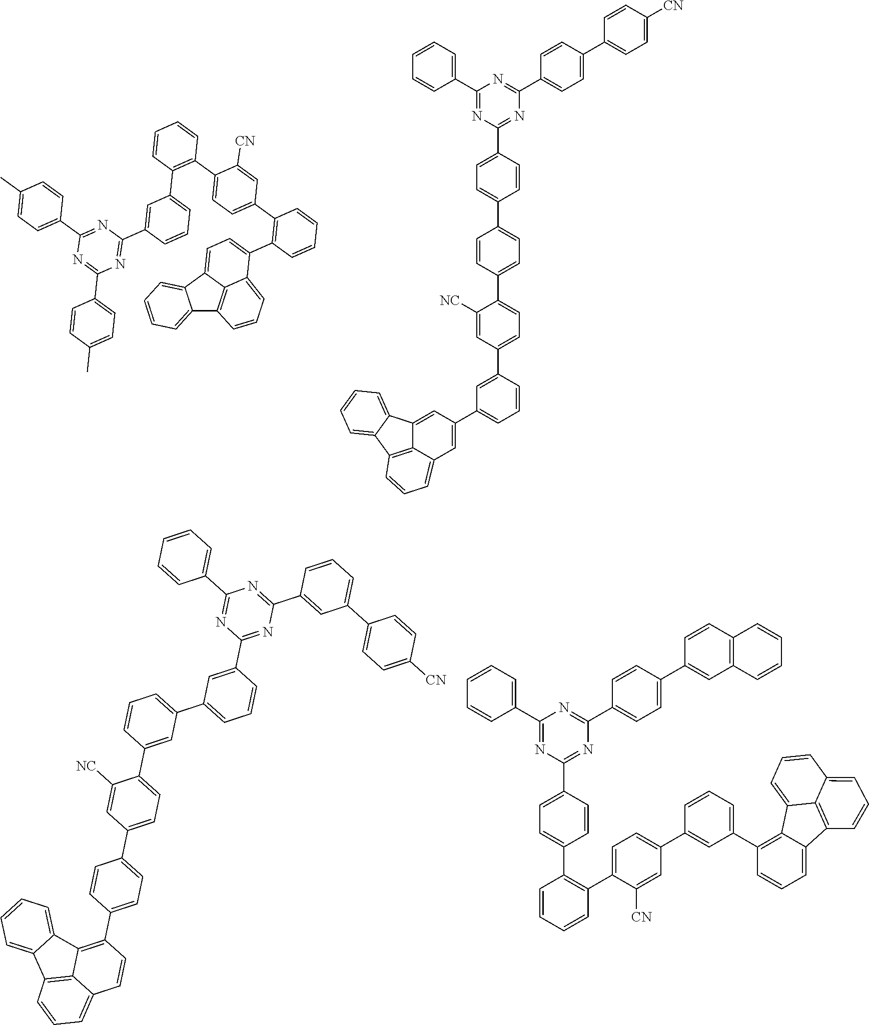

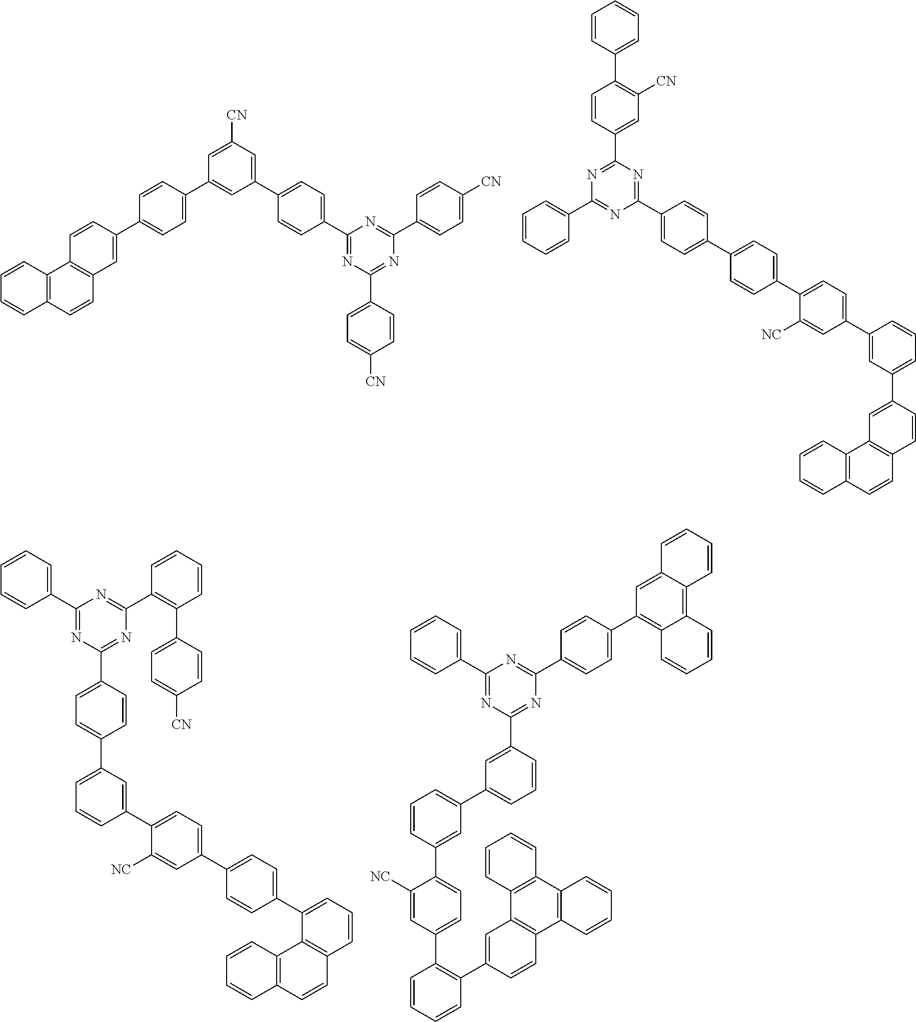 [SMILES: N#Cc1ccc(-c2nc(-c3ccc(C#N)cc3)nc(-c3ccc(-c4cc(C#N)cc(-c5ccc(-c6ccc7c(ccc8ccccc87)c6)cc5)c4)cc3)n2)cc1 ; N#Cc1cc(-c2cccc(-c3ccc4ccc5ccccc5c4c3)c2)ccc1-c1ccc(-c2ccc(-c3nc(-c4ccccc4)nc(-c4ccc(-c5ccccc5)c(C#N)c4)n3)cc2)cc1 ; N#Cc1ccc(-c2ccccc2-c2nc(-c3ccccc3)nc(-c3ccc(-c4cccc(-c5ccc(-c6ccc(-c7cccc8ccc9ccccc9c78)cc6)cc5C#N)c4)cc3)n2)cc1 ; N#Cc1cc(-c2ccccc2-c2ccc3c4ccccc4c4ccccc4c3c2)ccc1-c1cccc(-c2cccc(-c3nc(-c4ccccc4)nc(-c4ccc(-c5cc6ccccc6c6ccccc56)cc4)n3)c2)c1]

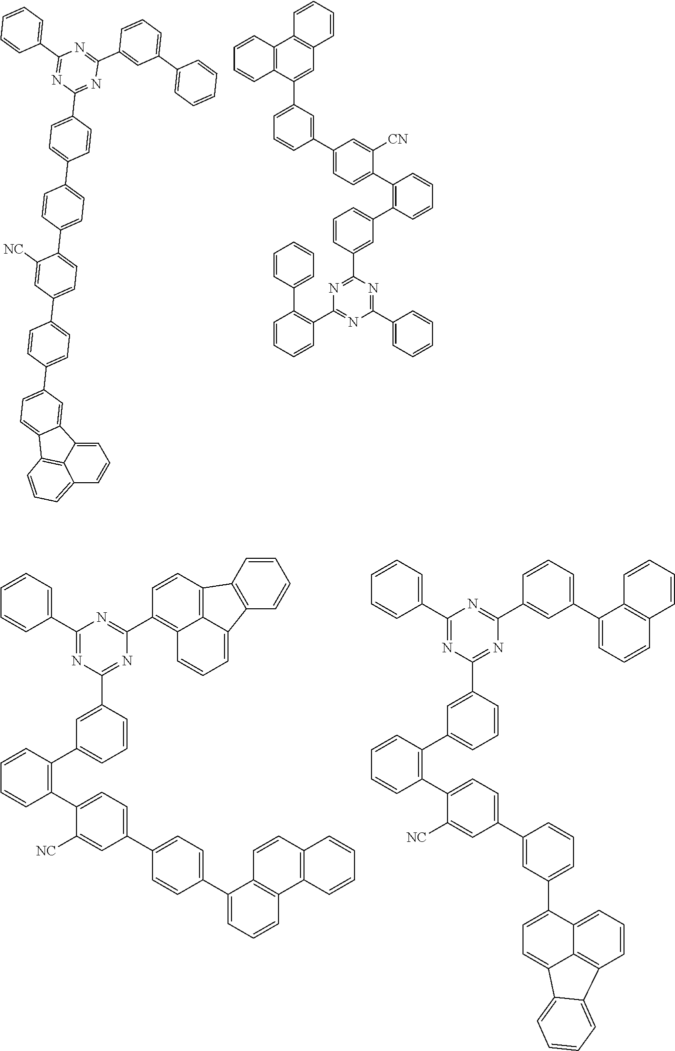

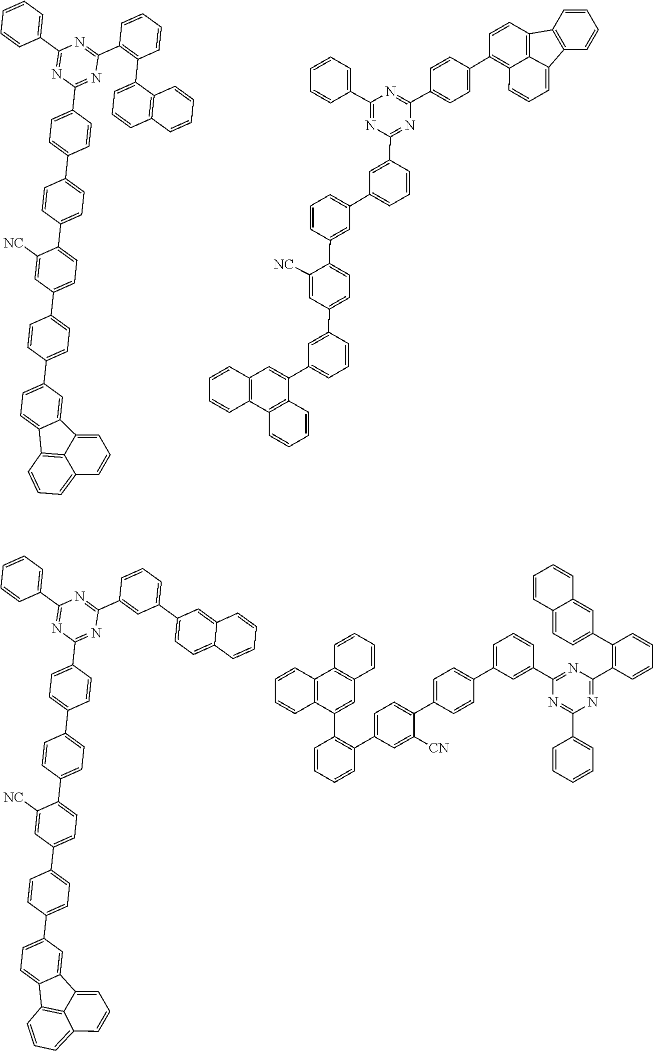 [SMILES: N#Cc1cc(-c2ccc(-c3ccc4c(c3)-c3cccc5cccc-4c35)cc2)ccc1-c1ccc(-c2ccc(-c3nc(-c4ccccc4)nc(-c4ccccc4-c4cccc5ccccc45)n3)cc2)cc1 ; N#Cc1cc(-c2cccc(-c3cc4ccccc4c4ccccc34)c2)ccc1-c1cccc(-c2cccc(-c3nc(-c4ccccc4)nc(-c4ccc(-c5ccc6c7c(cccc57)-c5ccccc5-6)cc4)n3)c2)c1 ; N#Cc1cc(-c2ccc(-c3ccc4c(c3)-c3cccc5cccc-4c35)cc2)ccc1-c1ccc(-c2ccc(-c3nc(-c4ccccc4)nc(-c4cccc(-c5ccc6ccccc6c5)c4)n3)cc2)cc1 ; N#Cc1cc(-c2ccccc2-c2cc3ccccc3c3ccccc23)ccc1-c1ccc(-c2cccc(-c3nc(-c4ccccc4)nc(-c4ccccc4-c4ccc5ccccc5c4)n3)c2)cc1]

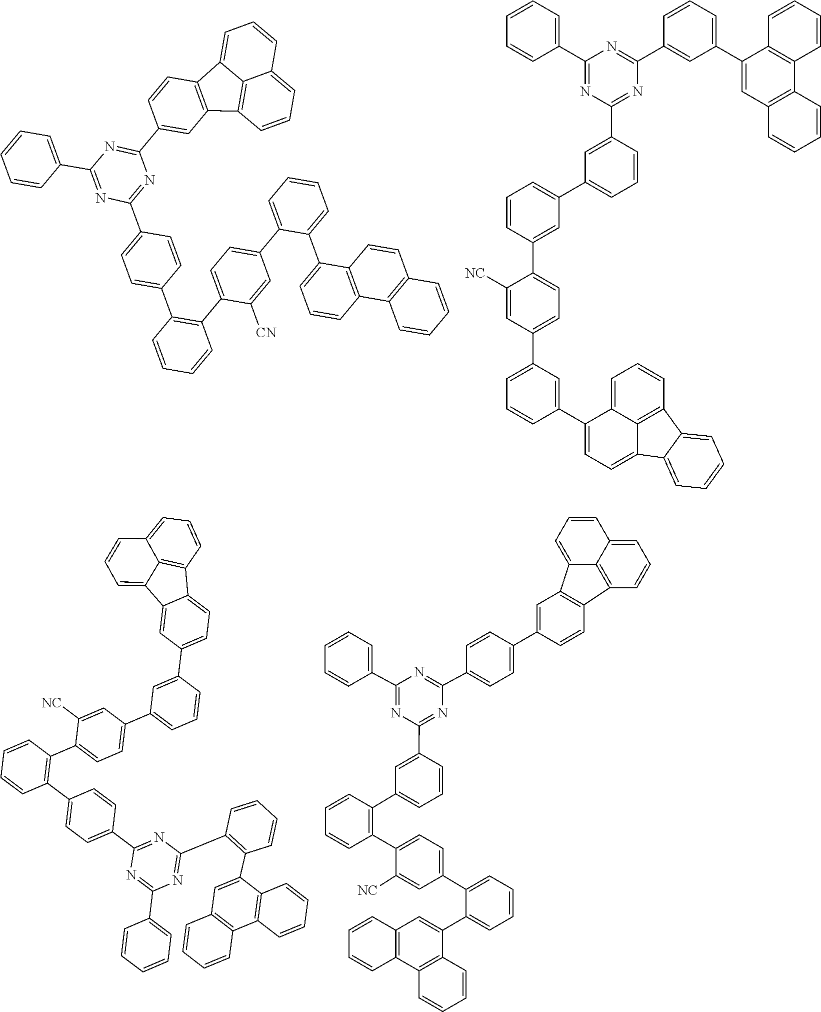

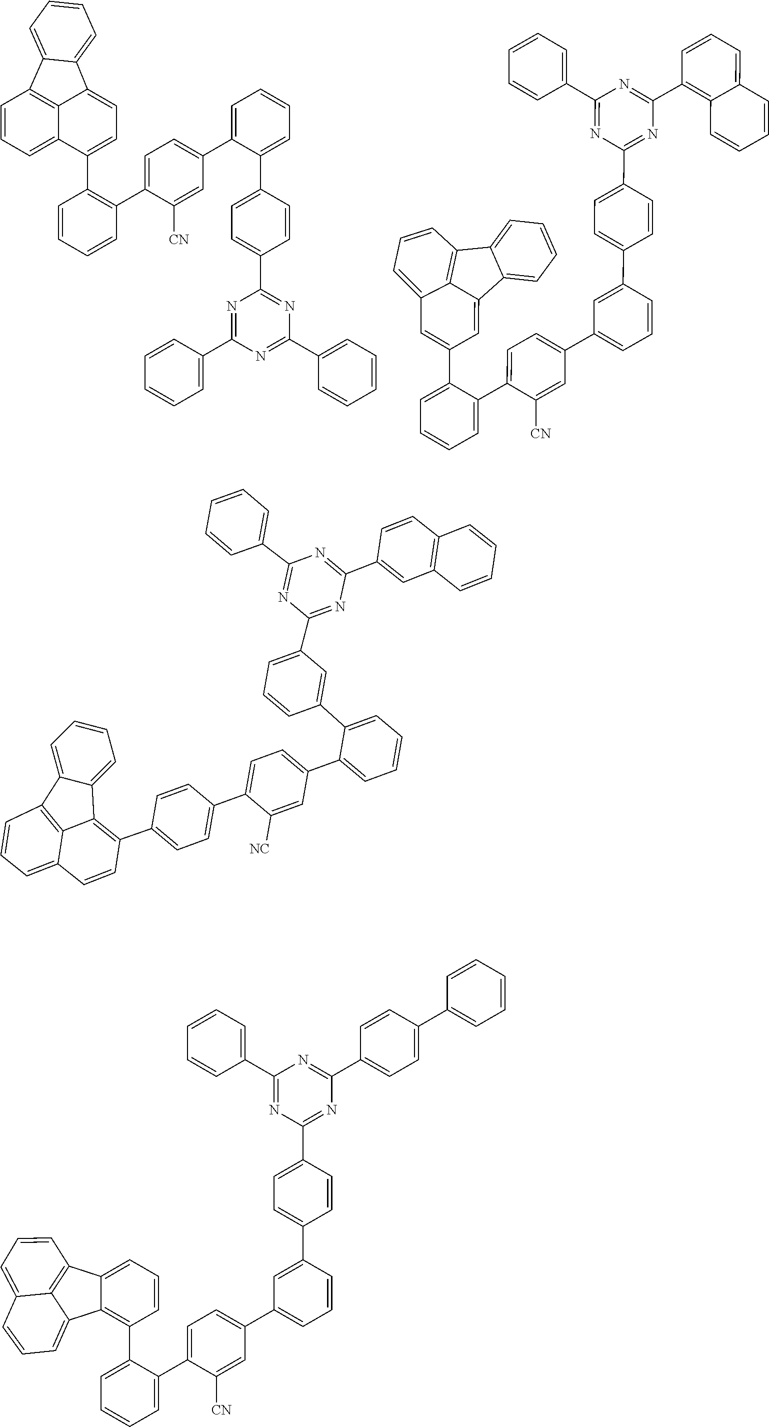

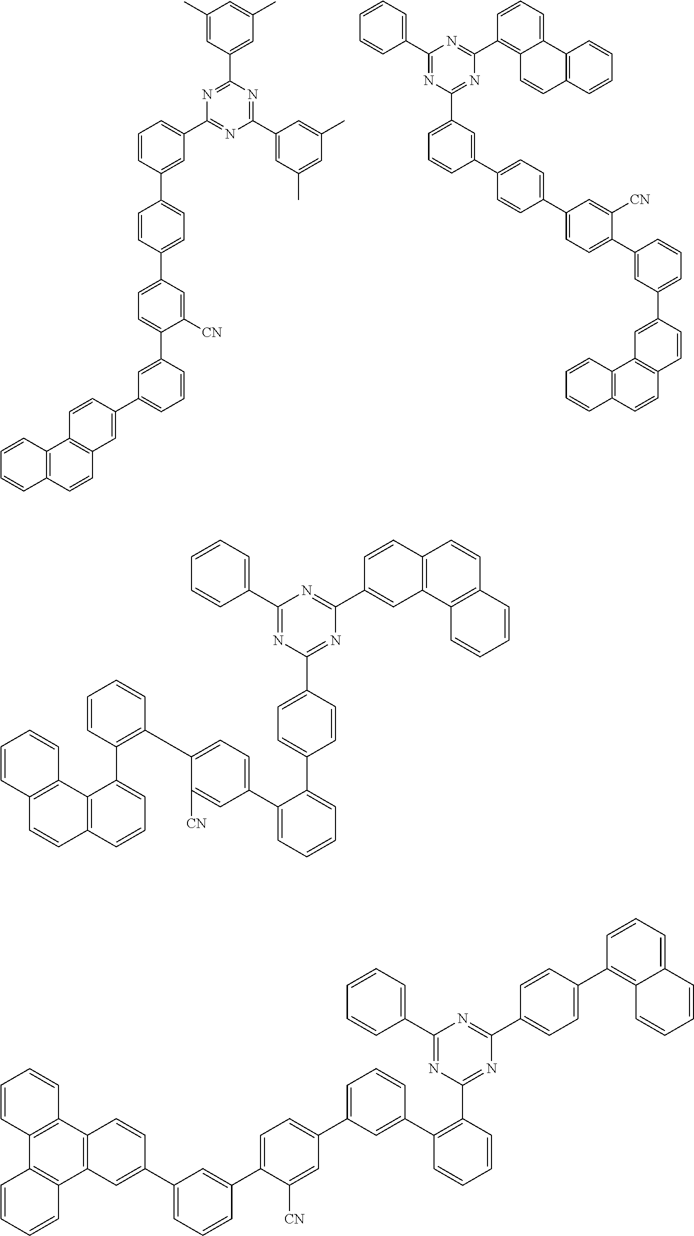

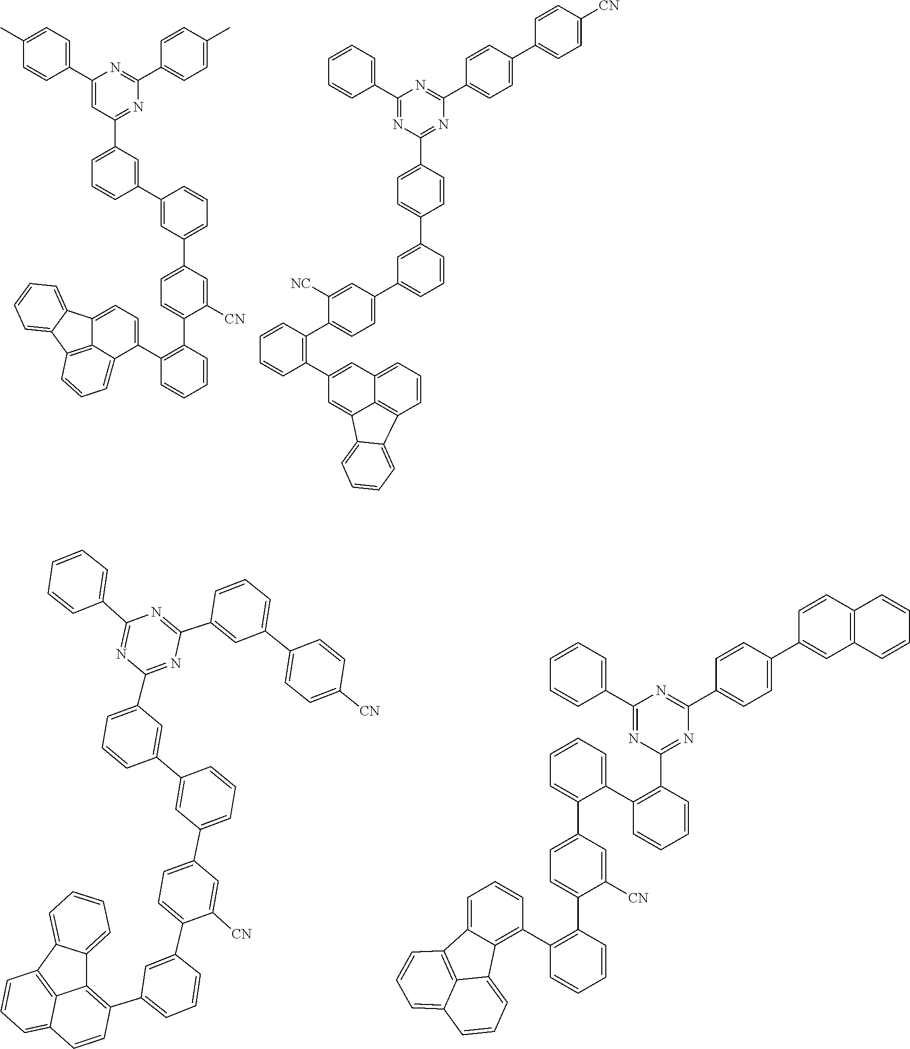

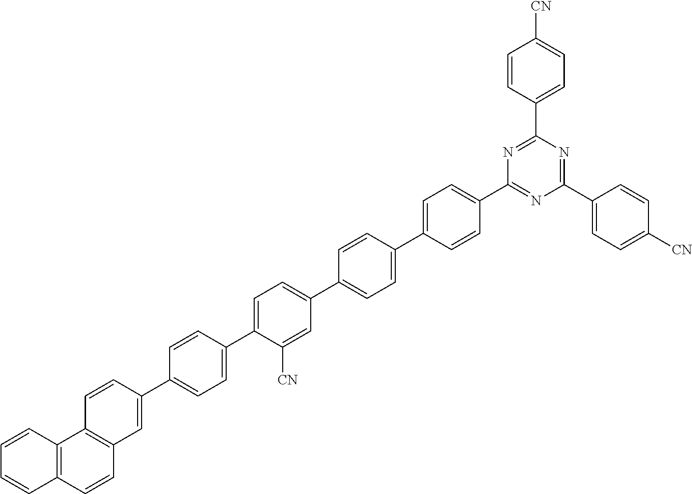 [SMILES: N#Cc1ccc(-c2nc(-c3ccc(C#N)cc3)nc(-c3ccc(-c4ccc(-c5ccc(-c6ccc(-c7ccc8c(ccc9ccccc98)c7)cc6)c(C#N)c5)cc4)cc3)n2)cc1]

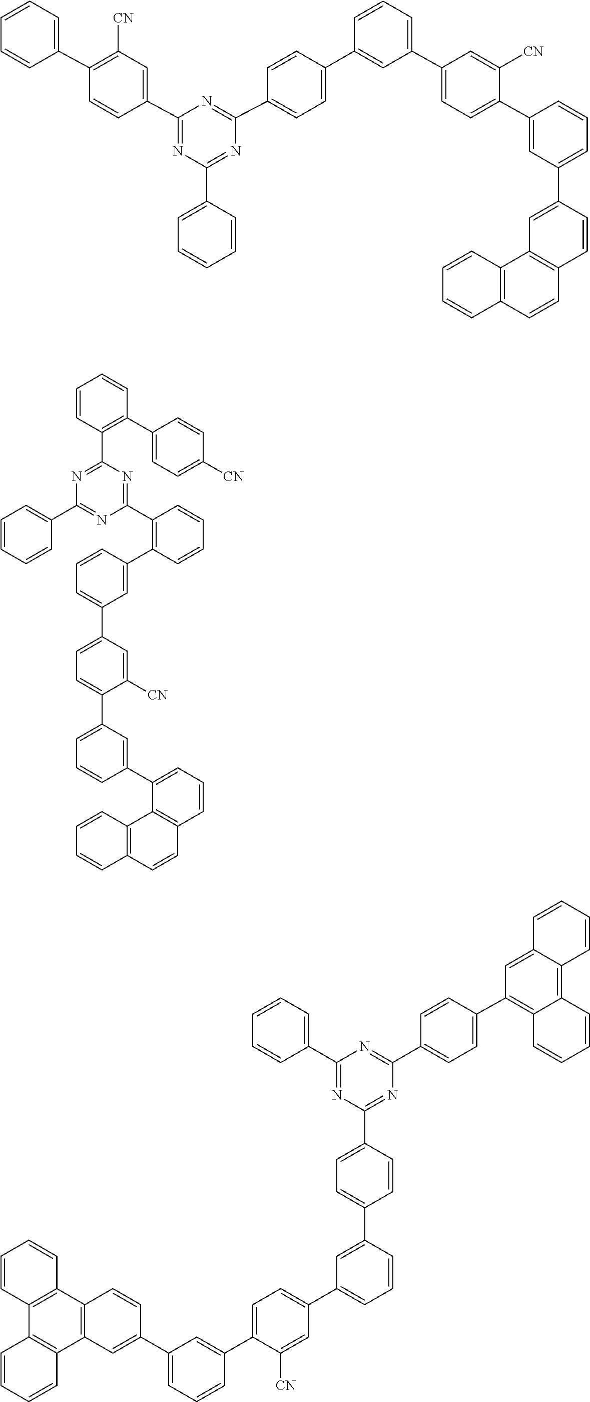

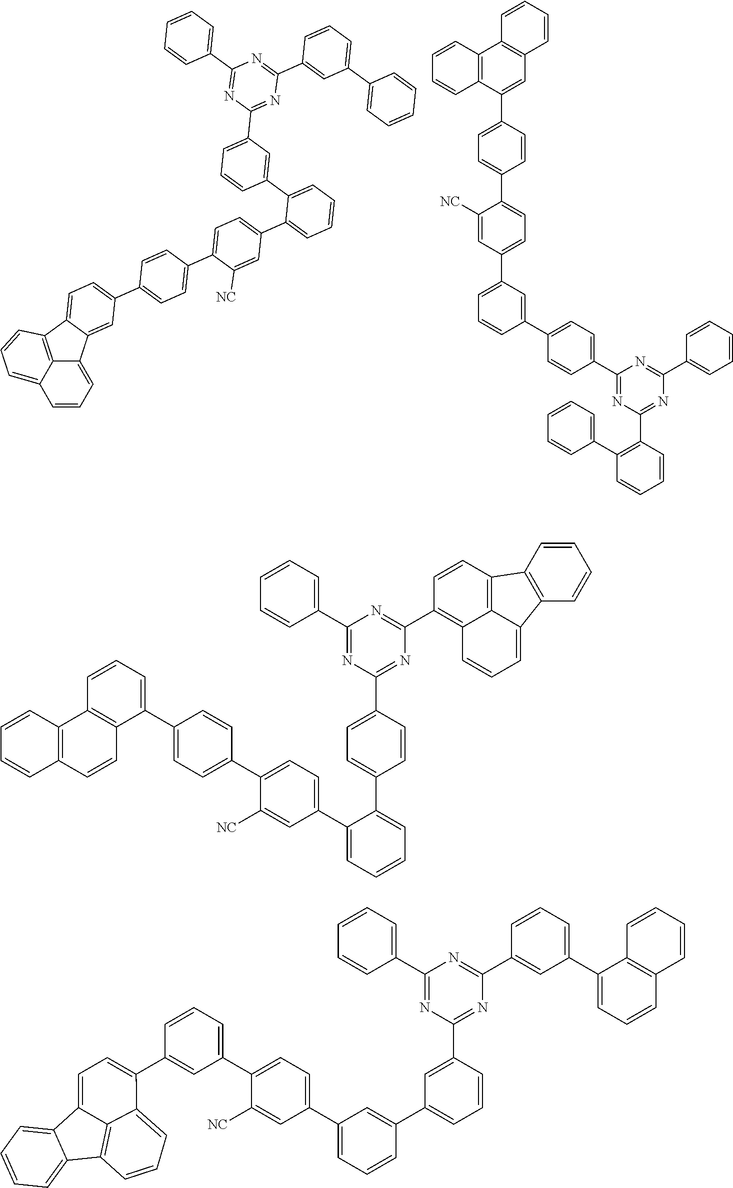

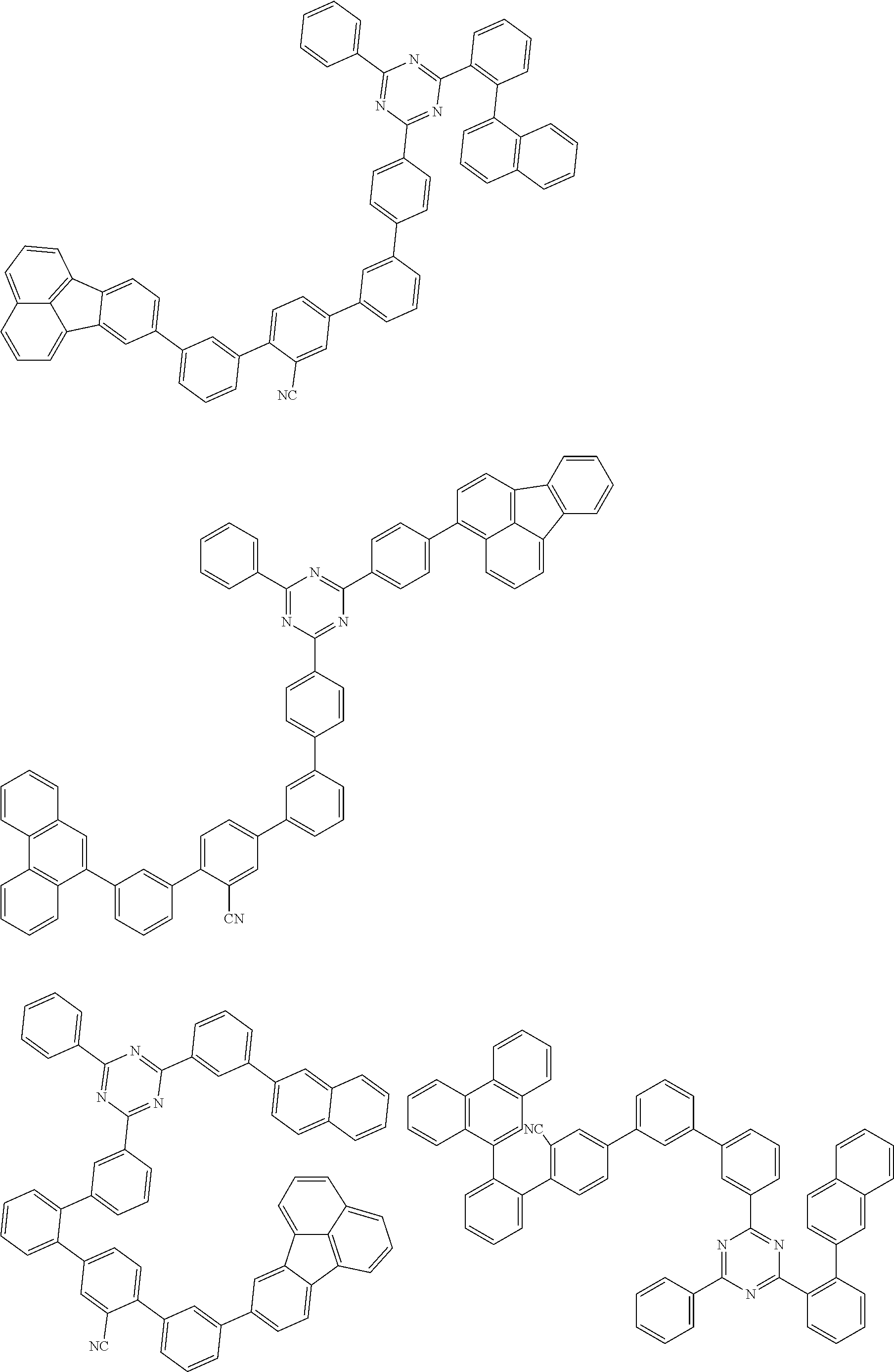

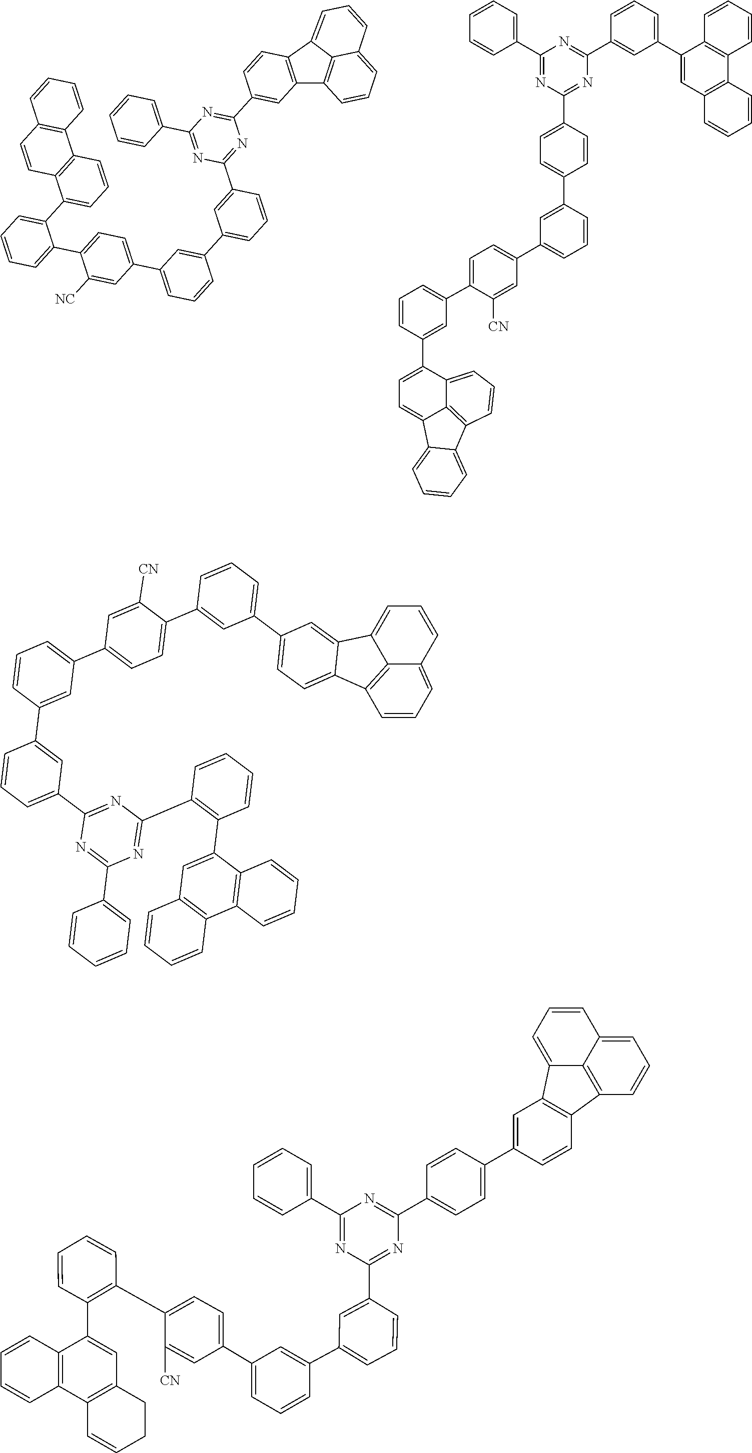

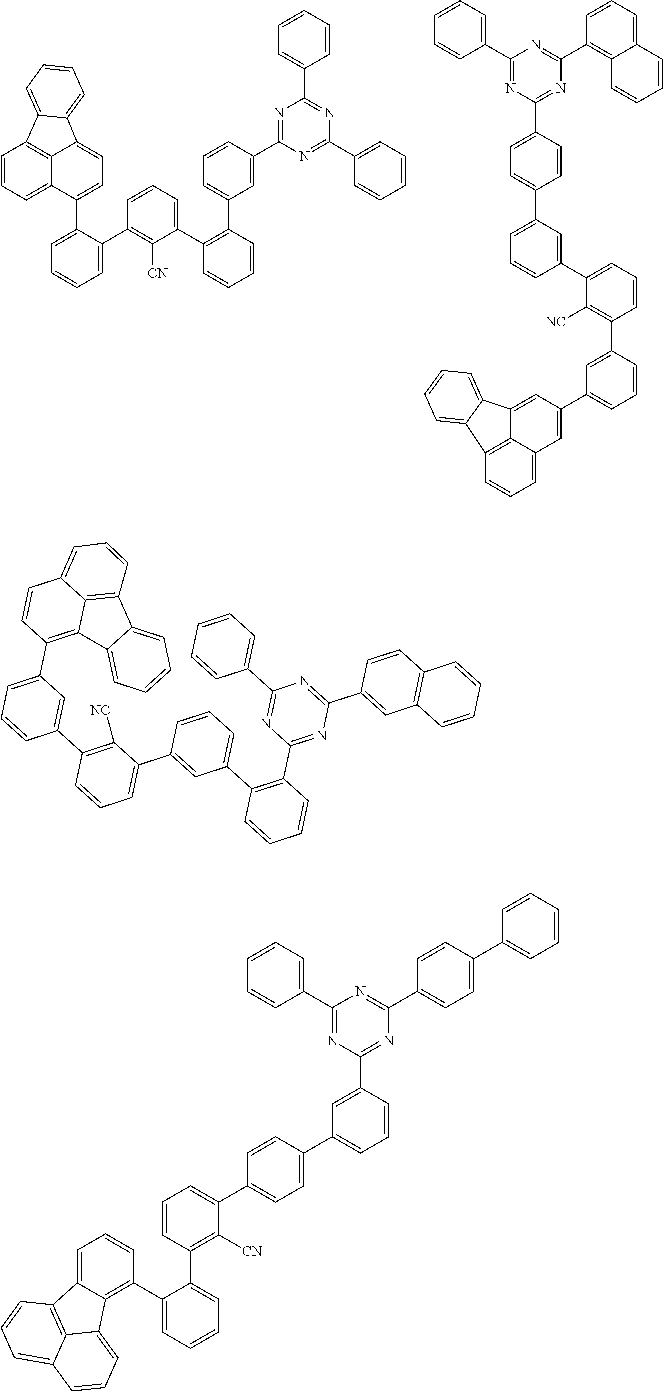 [SMILES: N#Cc1c(-c2ccccc2-c2cccc(-c3nc(-c4ccccc4)nc(-c4ccccc4)n3)c2)cccc1-c1ccccc1-c1ccc2c3c(cccc13)-c1ccccc1-2 ; N#Cc1c(-c2cccc(-c3ccc(-c4nc(-c5ccccc5)nc(-c5cccc6ccccc56)n4)cc3)c2)cccc1-c1cccc(-c2cc3c4c(cccc4c2)-c2ccccc2-3)c1 ; N#Cc1c(-c2cccc(-c3ccccc3-c3nc(-c4ccccc4)nc(-c4ccc5ccccc5c4)n3)c2)cccc1-c1cccc(-c2ccc3cccc4c3c2-c2ccccc2-4)c1 ; N#Cc1c(-c2ccc(-c3cccc(-c4nc(-c5ccccc5)nc(-c5ccc(-c6ccccc6)cc5)n4)c3)cc2)cccc1-c1ccccc1-c1cccc2c1-c1cccc3cccc-2c13]

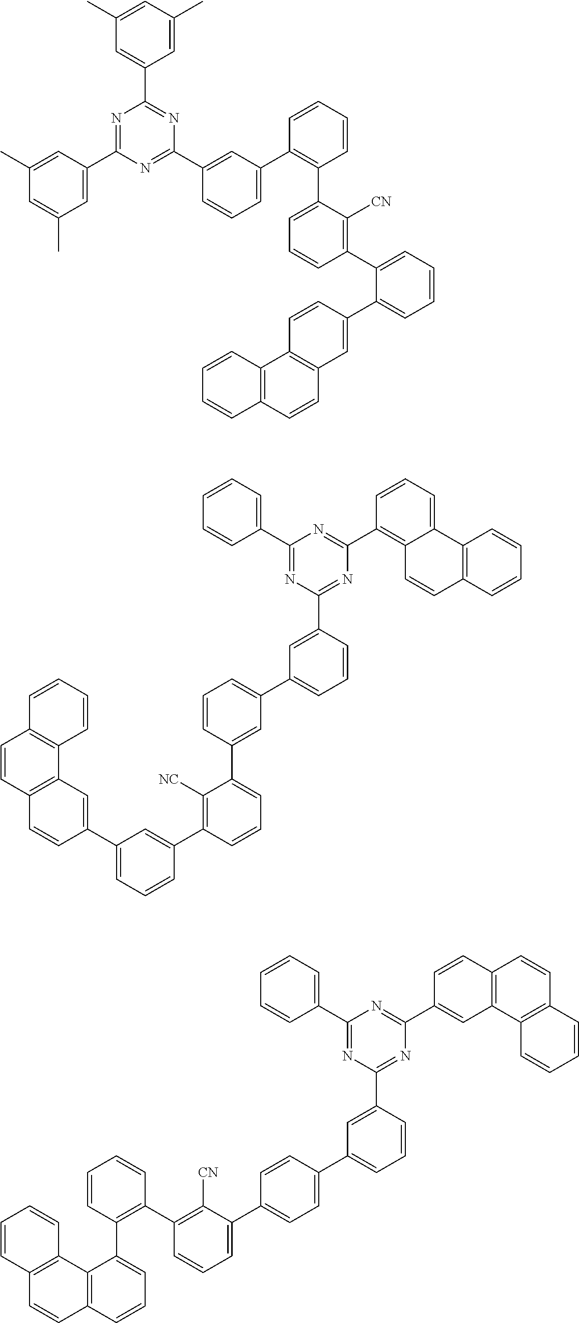 [SMILES: Cc1cc(C)cc(-c2nc(-c3cc(C)cc(C)c3)nc(-c3cccc(-c4ccccc4-c4cccc(-c5ccccc5-c5ccc6c(ccc7ccccc76)c5)c4C#N)c3)n2)c1 ; N#Cc1c(-c2cccc(-c3cccc(-c4nc(-c5ccccc5)nc(-c5cccc6c5ccc5ccccc56)n4)c3)c2)cccc1-c1cccc(-c2ccc3ccc4ccccc4c3c2)c1 ; N#Cc1c(-c2ccc(-c3cccc(-c4nc(-c5ccccc5)nc(-c5ccc6ccc7ccccc7c6c5)n4)c3)cc2)cccc1-c1ccccc1-c1cccc2ccc3ccccc3c12]

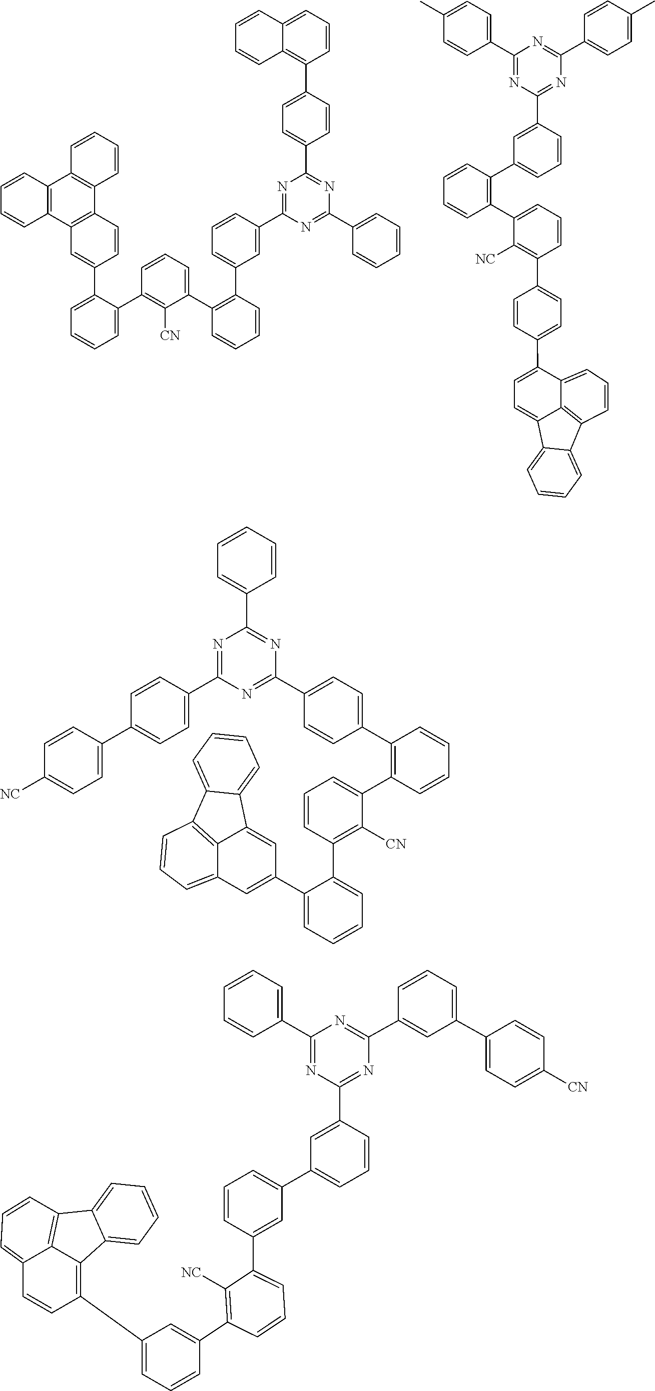

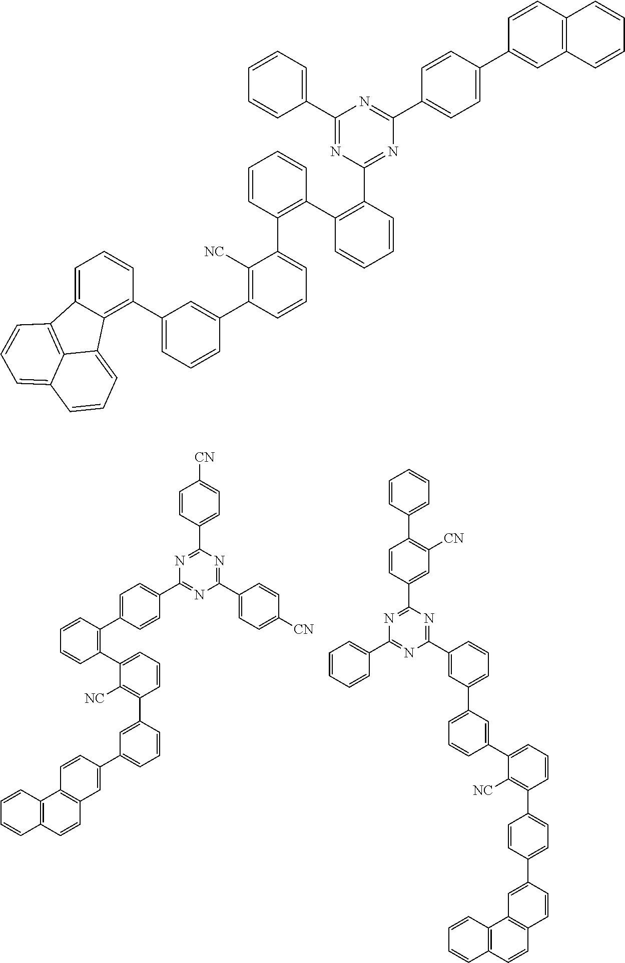 [SMILES: N#Cc1c(-c2cccc(-c3cccc4c3-c3cccc5cccc-4c35)c2)cccc1-c1ccccc1-c1ccccc1-c1nc(-c2ccccc2)nc(-c2ccc(-c3ccc4ccccc4c3)cc2)n1 ; N#Cc1ccc(-c2nc(-c3ccc(C#N)cc3)nc(-c3ccc(-c4ccccc4-c4cccc(-c5cccc(-c6ccc7c(ccc8ccccc87)c6)c5)c4C#N)cc3)n2)cc1 ; N#Cc1cc(-c2nc(-c3ccccc3)nc(-c3cccc(-c4cccc(-c5cccc(-c6ccc(-c7ccc8ccc9ccccc9c8c7)cc6)c5C#N)c4)c3)n2)ccc1-c1ccccc1]

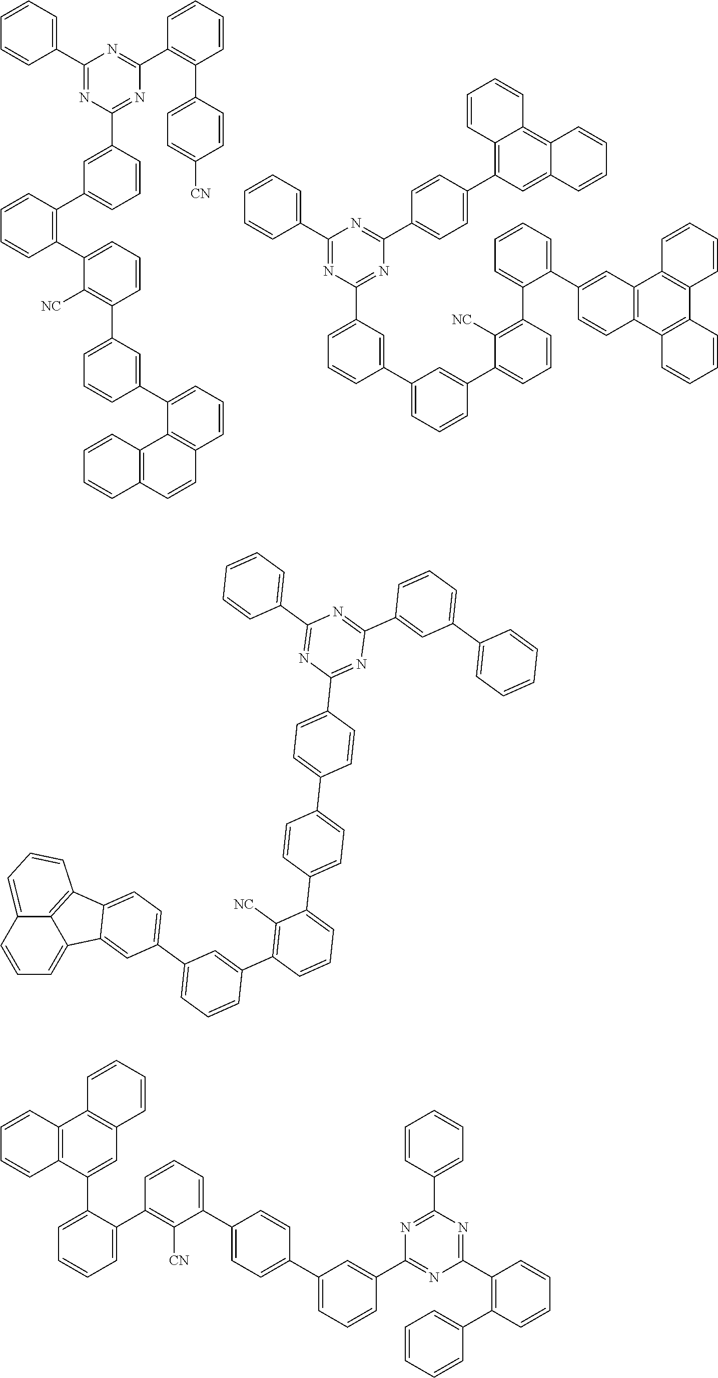

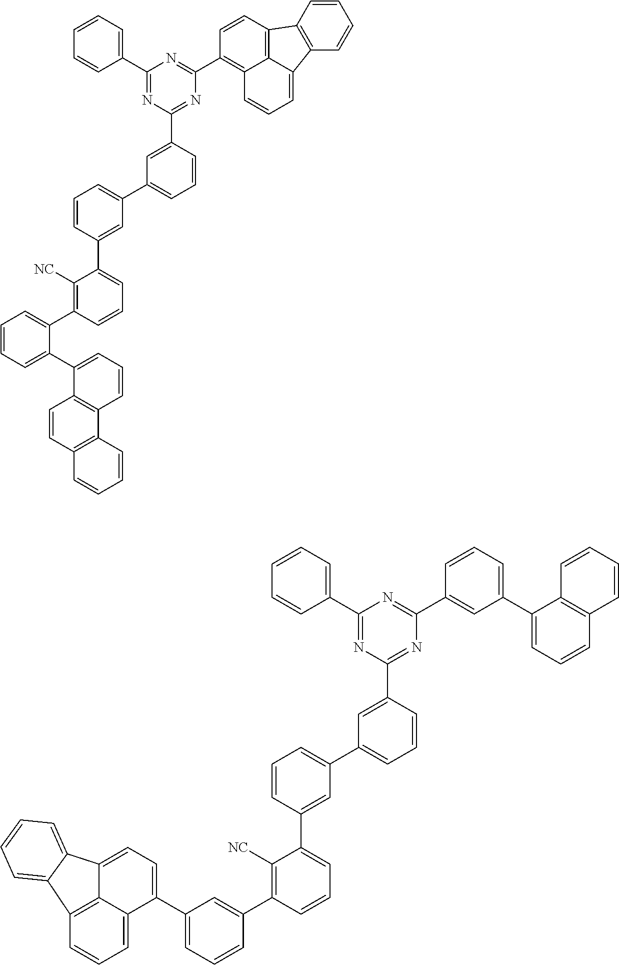 [SMILES: N#Cc1c(-c2cccc(-c3cccc(-c4nc(-c5ccccc5)nc(-c5ccc6c7c(cccc57)-c5ccccc5-6)n4)c3)c2)cccc1-c1ccccc1-c1cccc2c1ccc1ccccc12 ; N#Cc1c(-c2cccc(-c3cccc(-c4nc(-c5ccccc5)nc(-c5cccc(-c6cccc7ccccc67)c5)n4)c3)c2)cccc1-c1cccc(-c2ccc3c4c(cccc24)-c2ccccc2-3)c1]

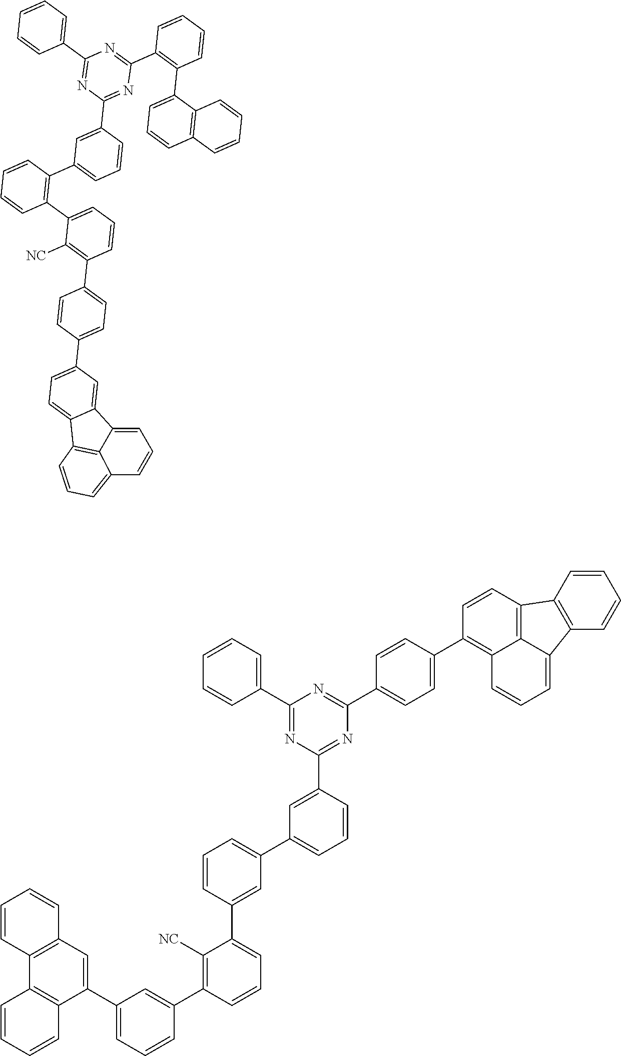 [SMILES: N#Cc1c(-c2ccc(-c3ccc4c(c3)-c3cccc5cccc-4c35)cc2)cccc1-c1ccccc1-c1cccc(-c2nc(-c3ccccc3)nc(-c3ccccc3-c3cccc4ccccc34)n2)c1 ; N#Cc1c(-c2cccc(-c3cccc(-c4nc(-c5ccccc5)nc(-c5ccc(-c6ccc7c8c(cccc68)-c6ccccc6-7)cc5)n4)c3)c2)cccc1-c1cccc(-c2cc3ccccc3c3ccccc23)c1]

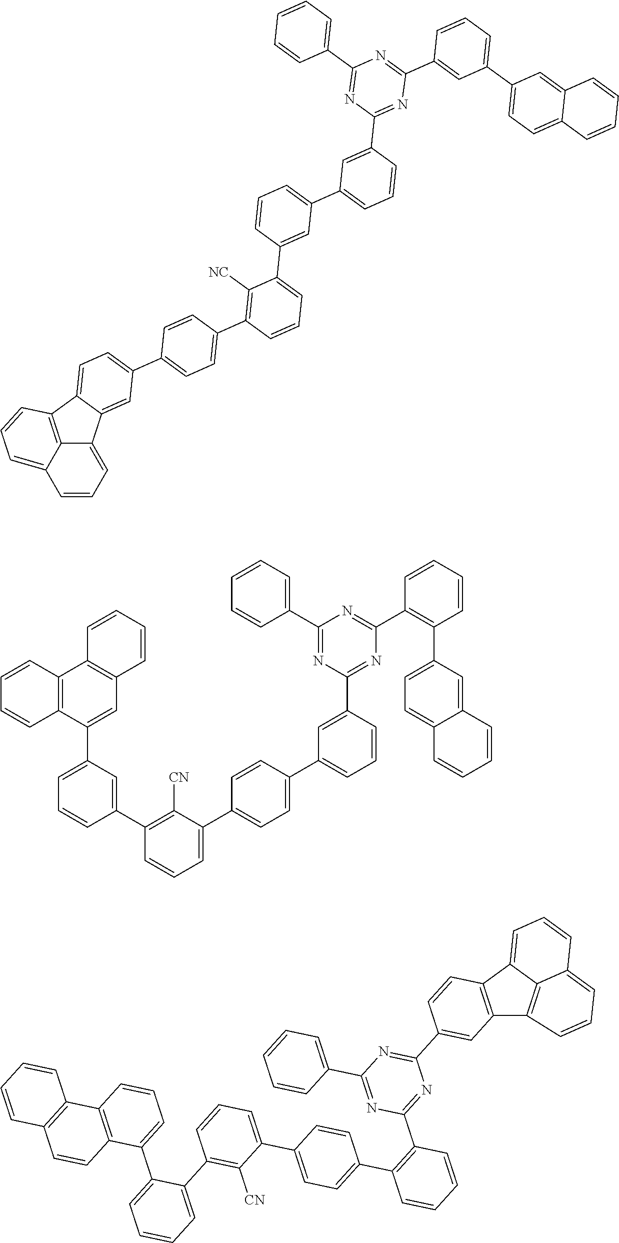 [SMILES: N#Cc1c(-c2ccc(-c3ccc4c(c3)-c3cccc5cccc-4c35)cc2)cccc1-c1cccc(-c2cccc(-c3nc(-c4ccccc4)nc(-c4cccc(-c5ccc6ccccc6c5)c4)n3)c2)c1 ; N#Cc1c(-c2ccc(-c3cccc(-c4nc(-c5ccccc5)nc(-c5ccccc5-c5ccc6ccccc6c5)n4)c3)cc2)cccc1-c1cccc(-c2cc3ccccc3c3ccccc23)c1 ; N#Cc1c(-c2ccc(-c3ccccc3-c3nc(-c4ccccc4)nc(-c4ccc5c(c4)-c4cccc6cccc-5c46)n3)cc2)cccc1-c1ccccc1-c1cccc2c1ccc1ccccc12]

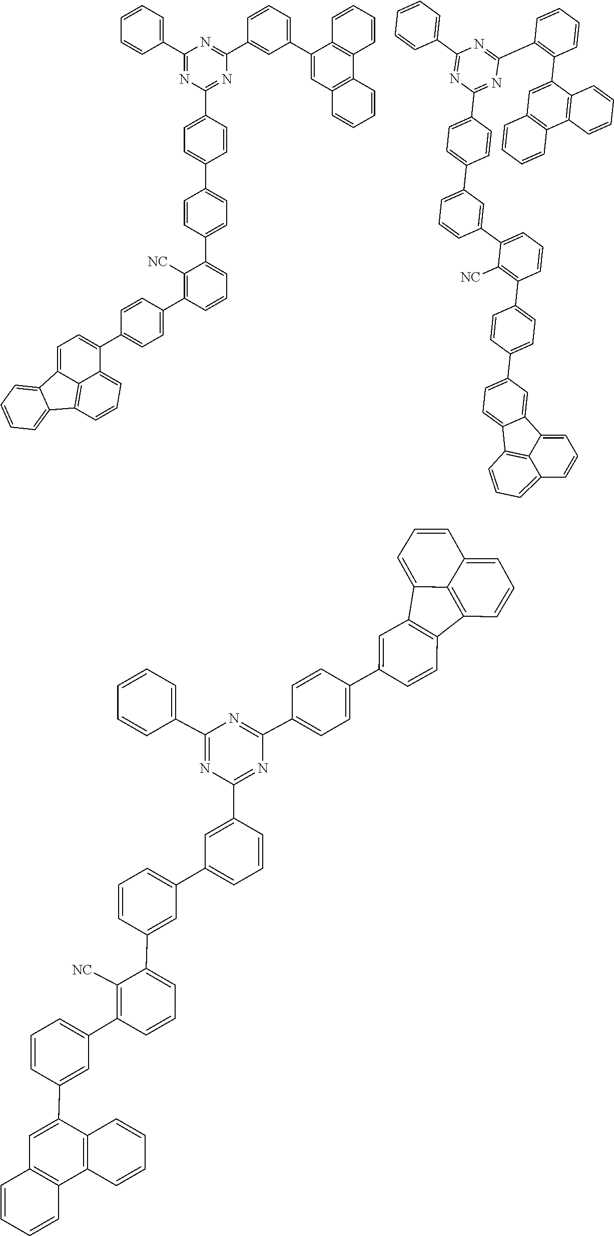

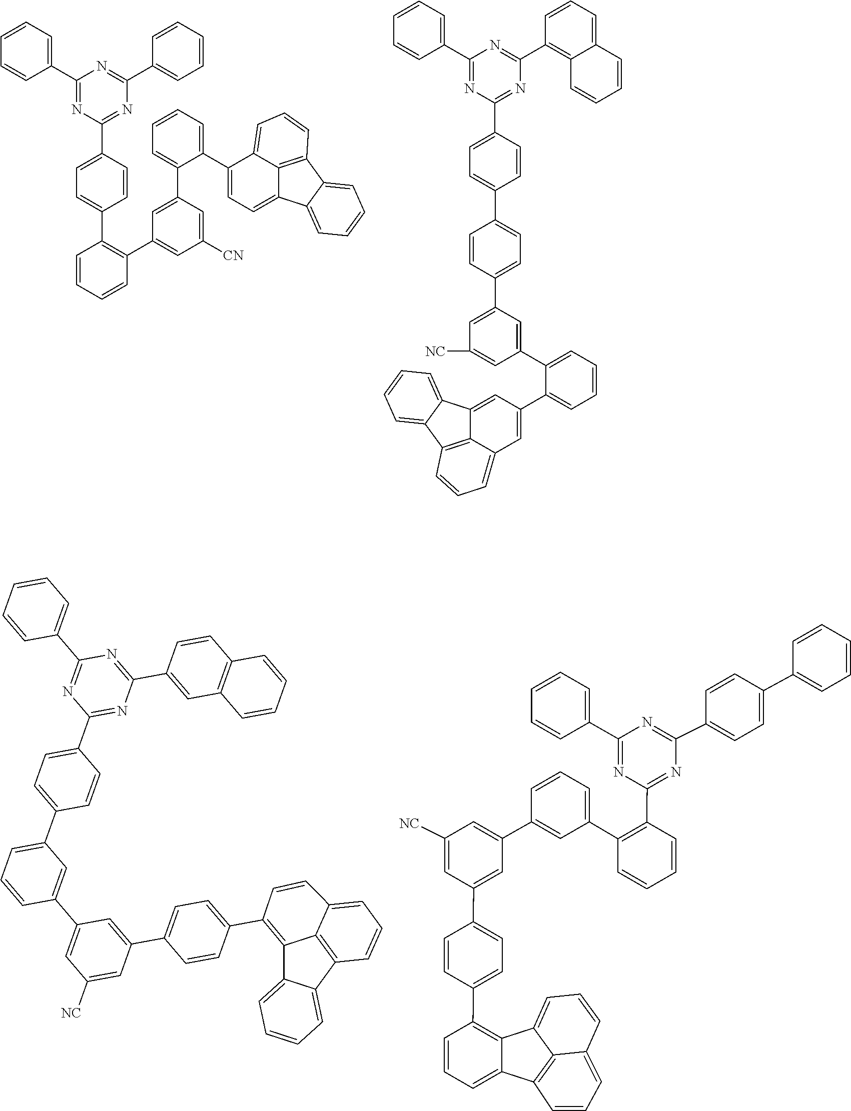 [SMILES: N#Cc1cc(-c2ccccc2-c2ccc(-c3nc(-c4ccccc4)nc(-c4ccccc4)n3)cc2)cc(-c2ccccc2-c2ccc3c4c(cccc24)-c2ccccc2-3)c1 ; N#Cc1cc(-c2ccc(-c3ccc(-c4nc(-c5ccccc5)nc(-c5cccc6ccccc56)n4)cc3)cc2)cc(-c2ccccc2-c2cc3c4c(cccc4c2)-c2ccccc2-3)c1 ; N#Cc1cc(-c2ccc(-c3ccc4cccc5c4c3-c3ccccc3-5)cc2)cc(-c2cccc(-c3ccc(-c4nc(-c5ccccc5)nc(-c5ccc6ccccc6c5)n4)cc3)c2)c1 ; N#Cc1cc(-c2ccc(-c3cccc4c3-c3cccc5cccc-4c35)cc2)cc(-c2cccc(-c3ccccc3-c3nc(-c4ccccc4)nc(-c4ccc(-c5ccccc5)cc4)n3)c2)c1]

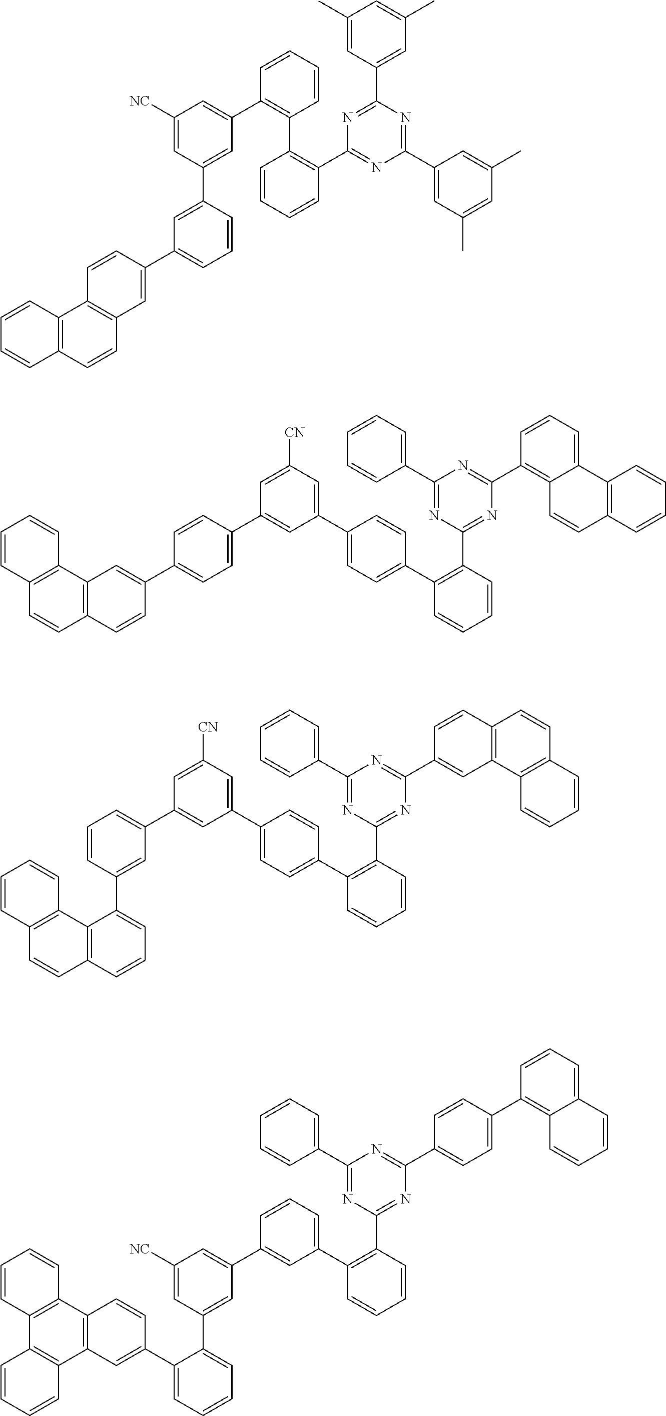 [SMILES: Cc1cc(C)cc(-c2nc(-c3cc(C)cc(C)c3)nc(-c3ccccc3-c3ccccc3-c3cc(C#N)cc(-c4cccc(-c5ccc6c(ccc7ccccc76)c5)c4)c3)n2)c1 ; N#Cc1cc(-c2ccc(-c3ccc4ccc5ccccc5c4c3)cc2)cc(-c2ccc(-c3ccccc3-c3nc(-c4ccccc4)nc(-c4cccc5c4ccc4ccccc45)n3)cc2)c1 ; N#Cc1cc(-c2ccc(-c3ccccc3-c3nc(-c4ccccc4)nc(-c4ccc5ccc6ccccc6c5c4)n3)cc2)cc(-c2cccc(-c3cccc4ccc5ccccc5c34)c2)c1 ; N#Cc1cc(-c2cccc(-c3ccccc3-c3nc(-c4ccccc4)nc(-c4ccc(-c5cccc6ccccc56)cc4)n3)c2)cc(-c2ccccc2-c2ccc3c4ccccc4c4ccccc4c3c2)c1]

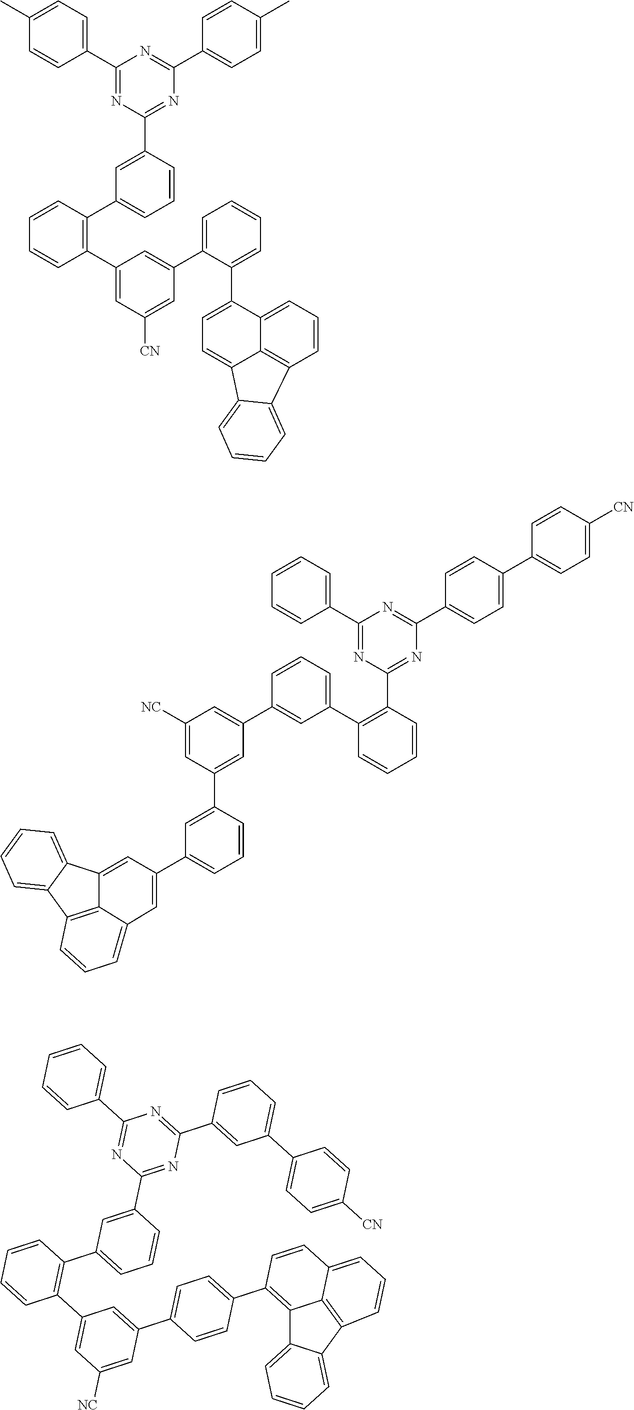 [SMILES: Cc1ccc(-c2nc(-c3ccc(C)cc3)nc(-c3cccc(-c4ccccc4-c4cc(C#N)cc(-c5ccccc5-c5ccc6c7c(cccc57)-c5ccccc5-6)c4)c3)n2)cc1 ; N#Cc1ccc(-c2ccc(-c3nc(-c4ccccc4)nc(-c4ccccc4-c4cccc(-c5cc(C#N)cc(-c6cccc(-c7cc8c9c(cccc9c7)-c7ccccc7-8)c6)c5)c4)n3)cc2)cc1 ; N#Cc1ccc(-c2cccc(-c3nc(-c4ccccc4)nc(-c4cccc(-c5ccccc5-c5cc(C#N)cc(-c6ccc(-c7ccc8cccc9c8c7-c7ccccc7-9)cc6)c5)c4)n3)c2)cc1]

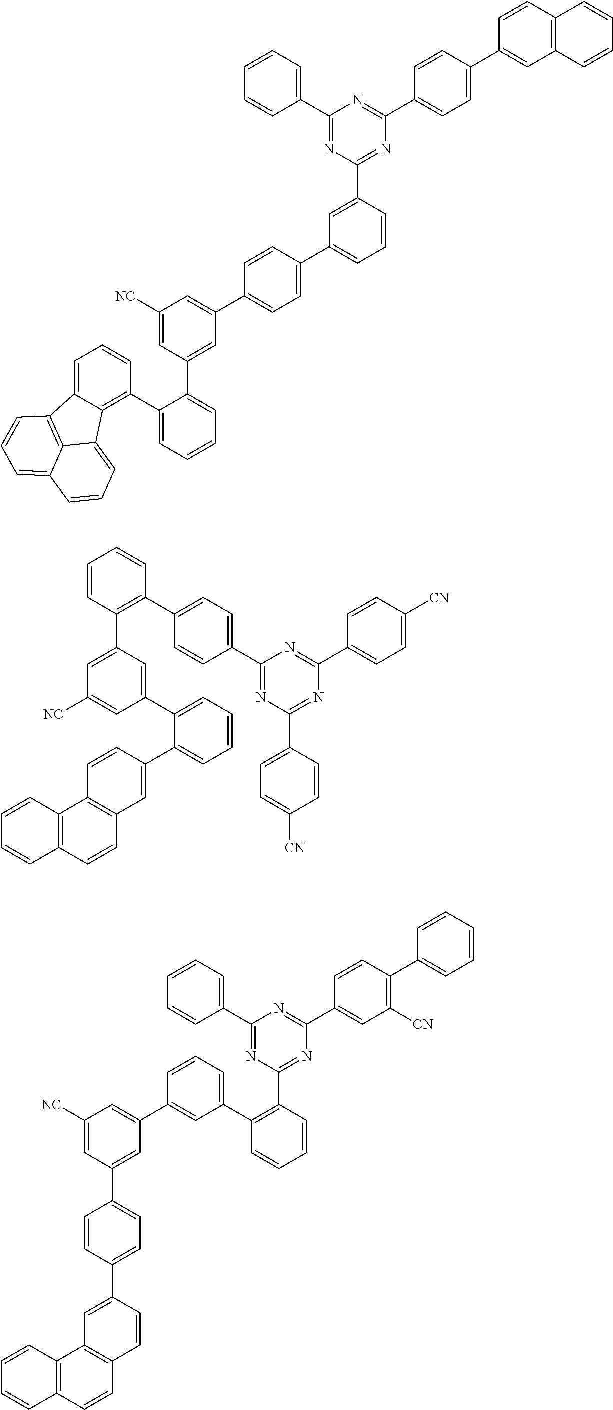 [SMILES: N#Cc1cc(-c2ccc(-c3cccc(-c4nc(-c5ccccc5)nc(-c5ccc(-c6ccc7ccccc7c6)cc5)n4)c3)cc2)cc(-c2ccccc2-c2cccc3c2-c2cccc4cccc-3c24)c1 ; N#Cc1ccc(-c2nc(-c3ccc(C#N)cc3)nc(-c3ccc(-c4ccccc4-c4cc(C#N)cc(-c5ccccc5-c5ccc6c(ccc7ccccc76)c5)c4)cc3)n2)cc1 ; N#Cc1cc(-c2ccc(-c3ccc4ccc5ccccc5c4c3)cc2)cc(-c2cccc(-c3ccccc3-c3nc(-c4ccccc4)nc(-c4ccc(-c5ccccc5)c(C#N)c4)n3)c2)c1]

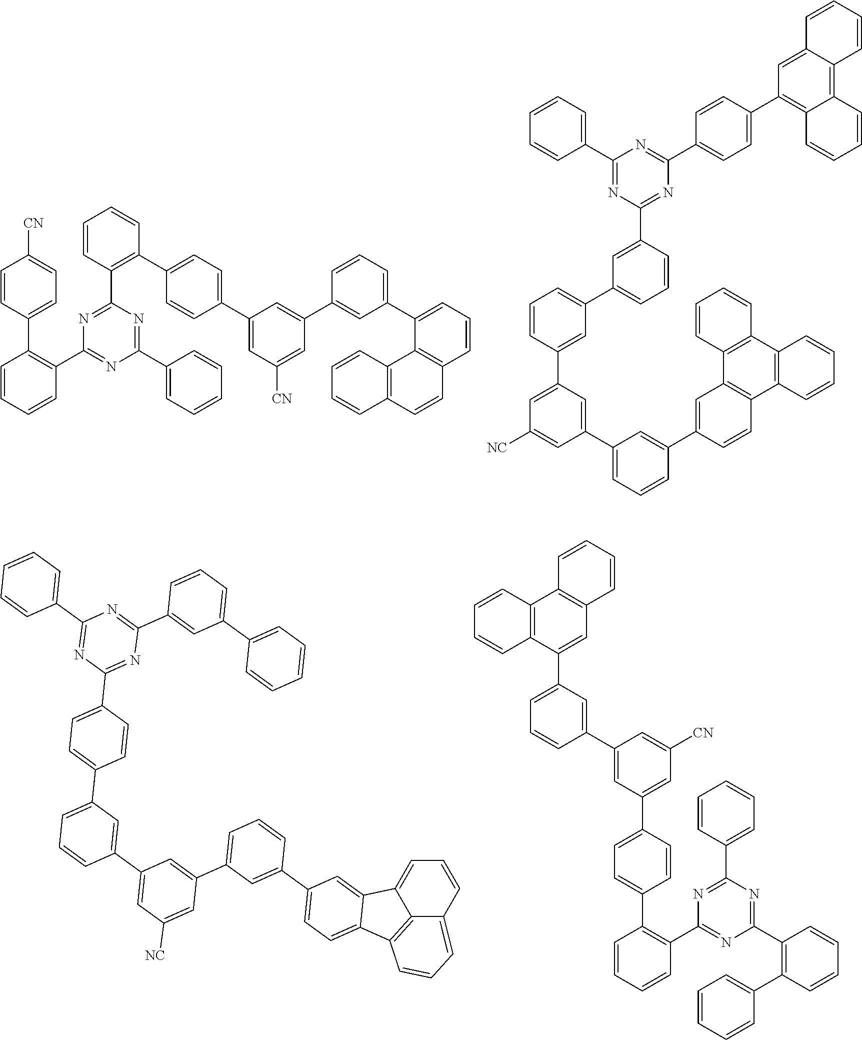 [SMILES: N#Cc1ccc(-c2ccccc2-c2nc(-c3ccccc3)nc(-c3ccccc3-c3ccc(-c4cc(C#N)cc(-c5cccc(-c6cccc7ccc8ccccc8c67)c5)c4)cc3)n2)cc1 ; N#Cc1cc(-c2cccc(-c3cccc(-c4nc(-c5ccccc5)nc(-c5ccc(-c6cc7ccccc7c7ccccc67)cc5)n4)c3)c2)cc(-c2cccc(-c3ccc4c5ccccc5c5ccccc5c4c3)c2)c1 ; N#Cc1cc(-c2cccc(-c3ccc(-c4nc(-c5ccccc5)nc(-c5cccc(-c6ccccc6)c5)n4)cc3)c2)cc(-c2cccc(-c3ccc4c(c3)-c3cccc5cccc-4c35)c2)c1 ; N#Cc1cc(-c2ccc(-c3ccccc3-c3nc(-c4ccccc4)nc(-c4ccccc4-c4ccccc4)n3)cc2)cc(-c2cccc(-c3cc4ccccc4c4ccccc34)c2)c1]

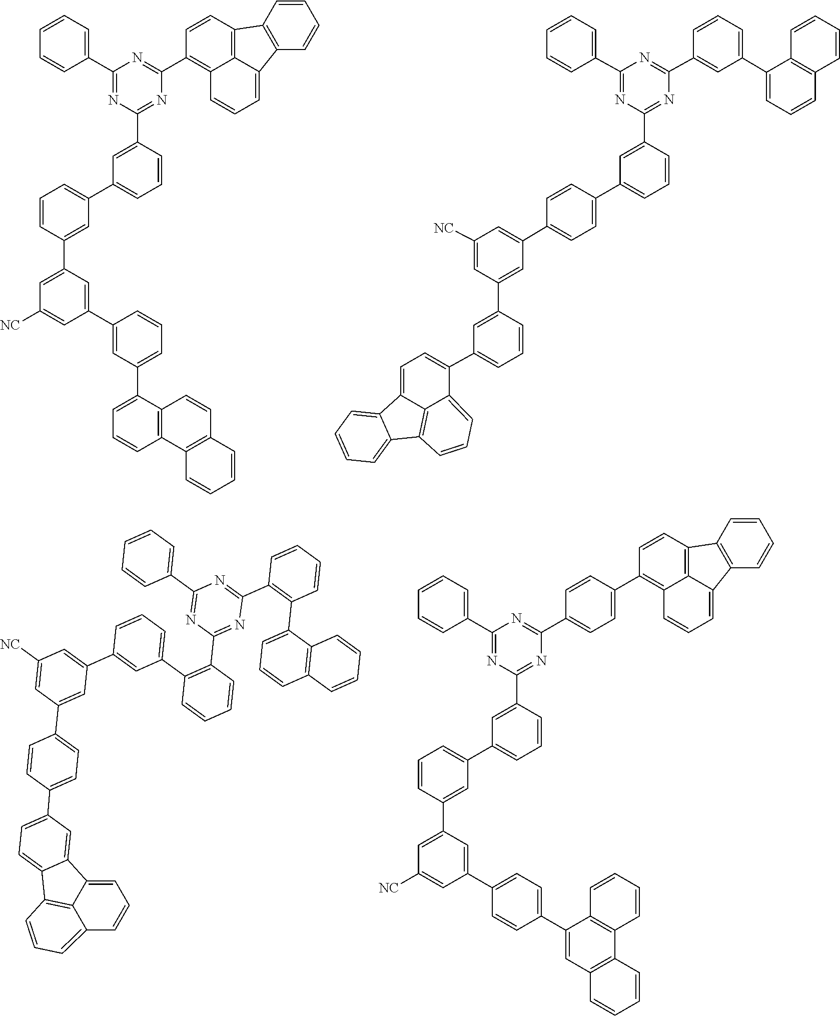

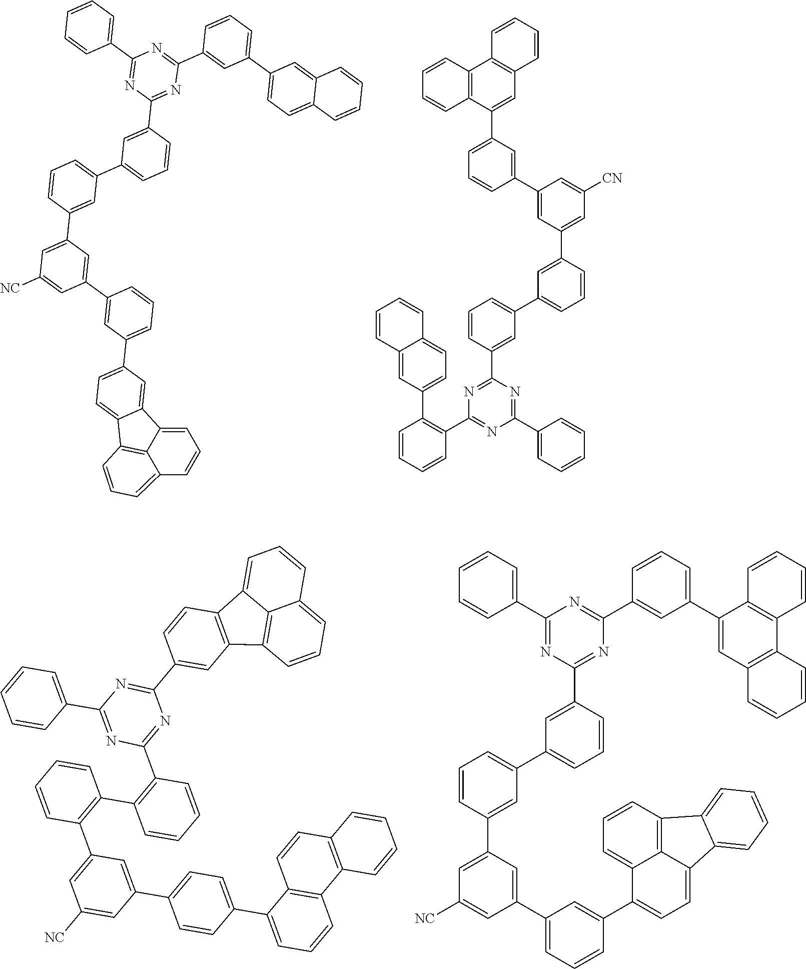 [SMILES: N#Cc1cc(-c2cccc(-c3cccc(-c4nc(-c5ccccc5)nc(-c5cccc(-c6ccc7ccccc7c6)c5)n4)c3)c2)cc(-c2cccc(-c3ccc4c(c3)-c3cccc5cccc-4c35)c2)c1 ; N#Cc1cc(-c2cccc(-c3cccc(-c4nc(-c5ccccc5)nc(-c5ccccc5-c5ccc6ccccc6c5)n4)c3)c2)cc(-c2cccc(-c3cc4ccccc4c4ccccc34)c2)c1 ; N#Cc1cc(-c2ccc(-c3cccc4c3ccc3ccccc34)cc2)cc(-c2ccccc2-c2ccccc2-c2nc(-c3ccccc3)nc(-c3ccc4c(c3)-c3cccc5cccc-4c35)n2)c1 ; N#Cc1cc(-c2cccc(-c3cccc(-c4nc(-c5ccccc5)nc(-c5cccc(-c6cc7ccccc7c7ccccc67)c5)n4)c3)c2)cc(-c2cccc(-c3ccc4c5c(cccc35)-c3ccccc3-4)c2)c1]

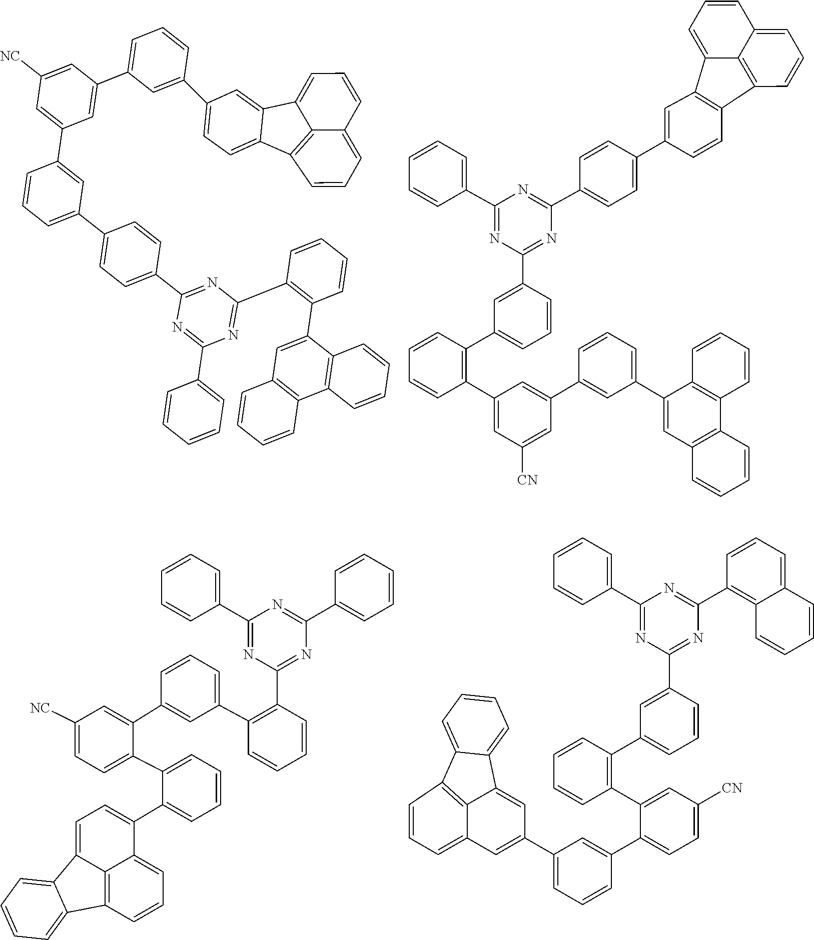

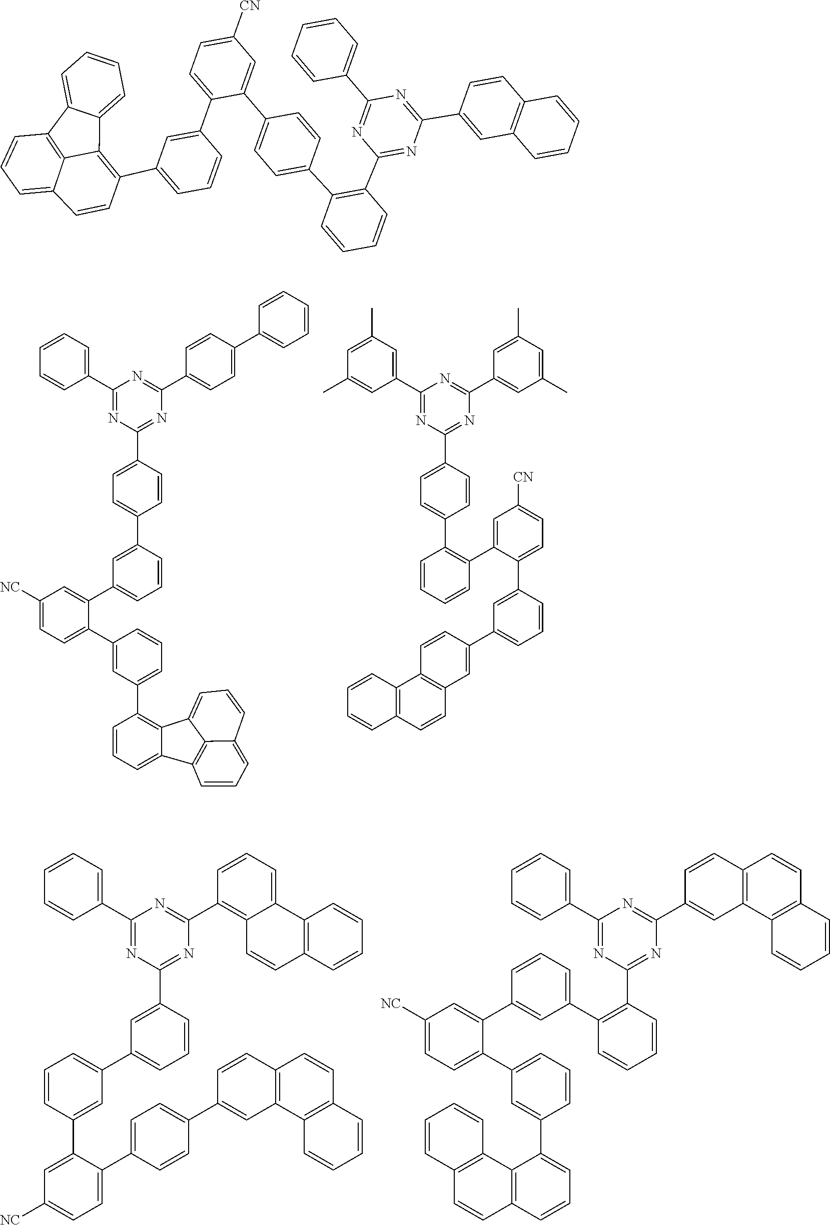

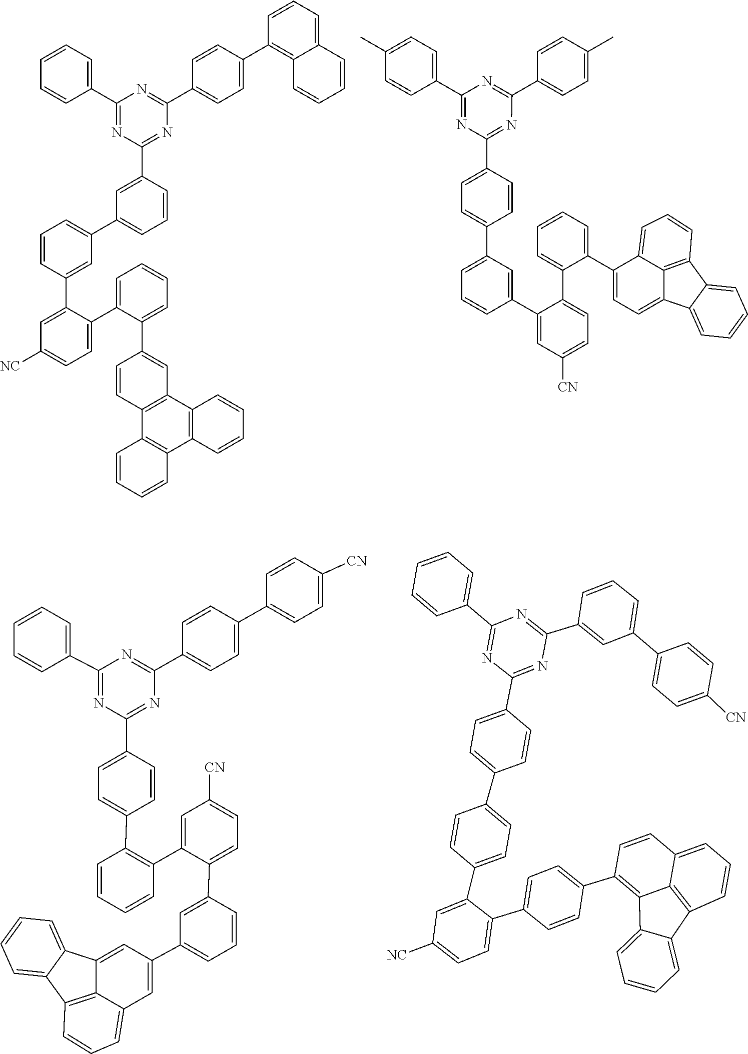

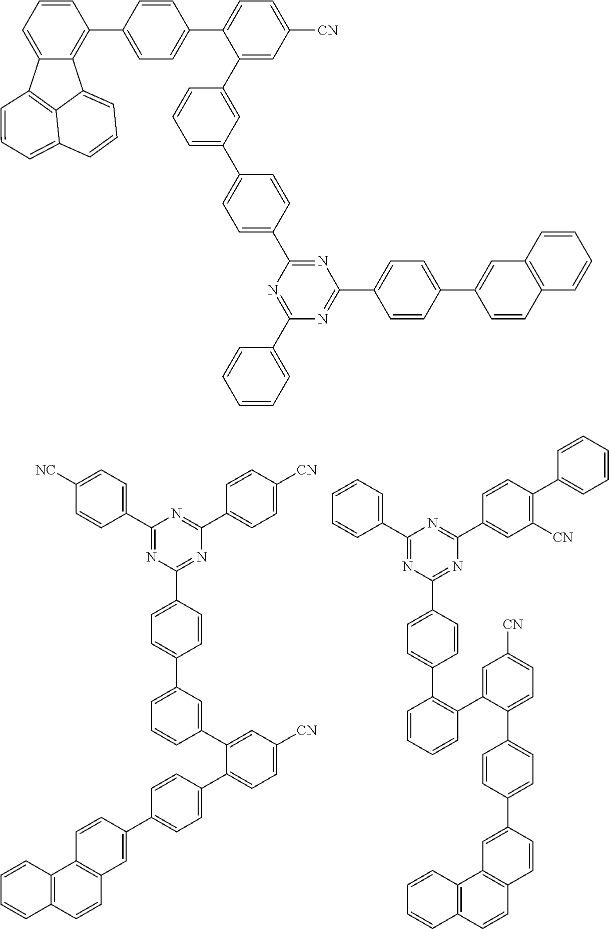

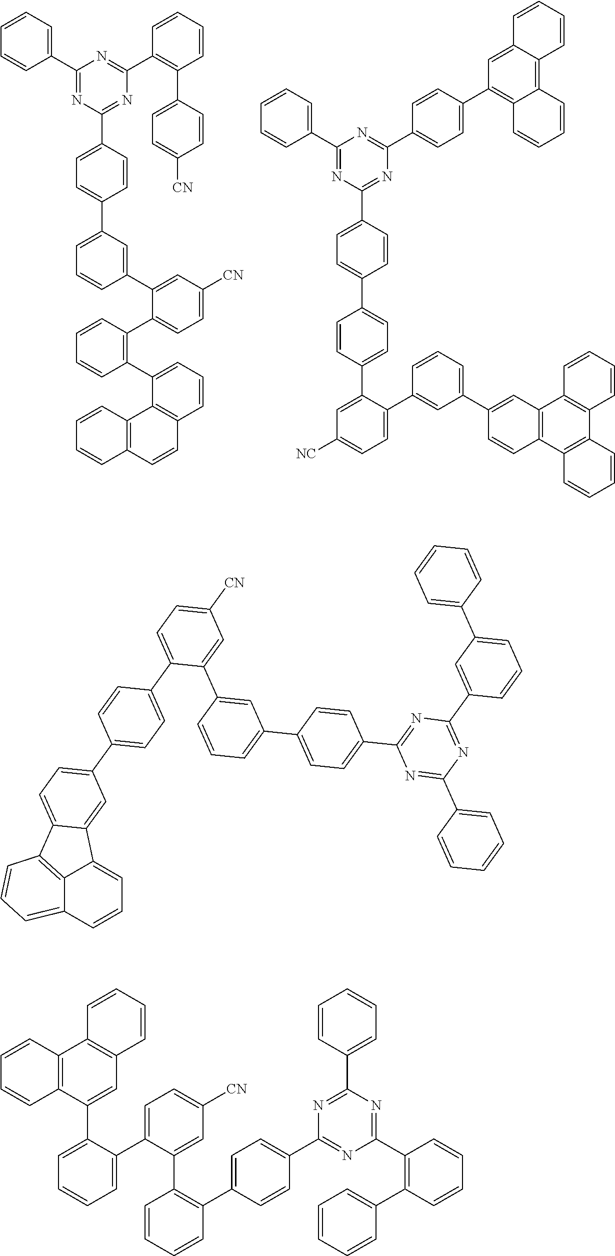 [SMILES: N#Cc1ccc(-c2ccccc2-c2nc(-c3ccccc3)nc(-c3ccc(-c4cccc(-c5cc(C#N)ccc5-c5ccccc5-c5cccc6ccc7ccccc7c56)c4)cc3)n2)cc1 ; N#Cc1ccc(-c2cccc(-c3ccc4c5ccccc5c5ccccc5c4c3)c2)c(-c2ccc(-c3ccc(-c4nc(-c5ccccc5)nc(-c5ccc(-c6cc7ccccc7c7ccccc67)cc5)n4)cc3)cc2)c1 ; N#Cc1ccc(-c2ccc(-c3ccc4c(c3)-c3cccc5cccc-4c35)cc2)c(-c2cccc(-c3ccc(-c4nc(-c5ccccc5)nc(-c5cccc(-c6ccccc6)c5)n4)cc3)c2)c1 ; N#Cc1ccc(-c2ccccc2-c2cc3ccccc3c3ccccc23)c(-c2ccccc2-c2ccc(-c3nc(-c4ccccc4)nc(-c4ccccc4-c4ccccc4)n3)cc2)c1]

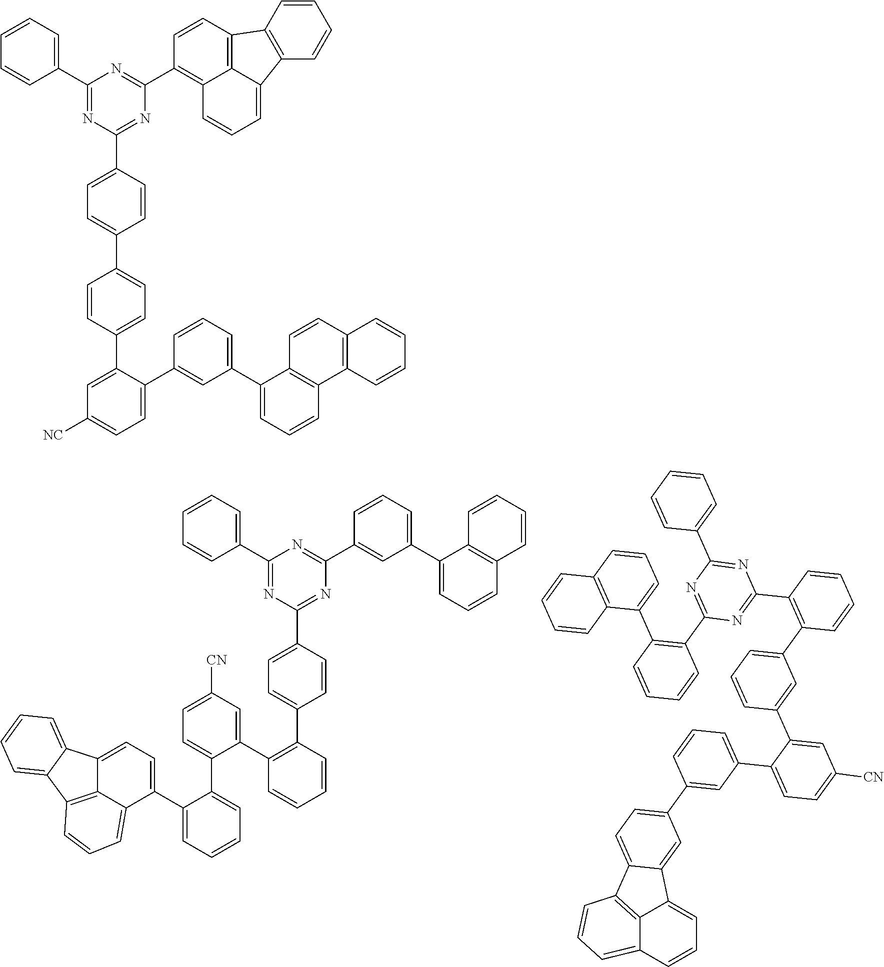

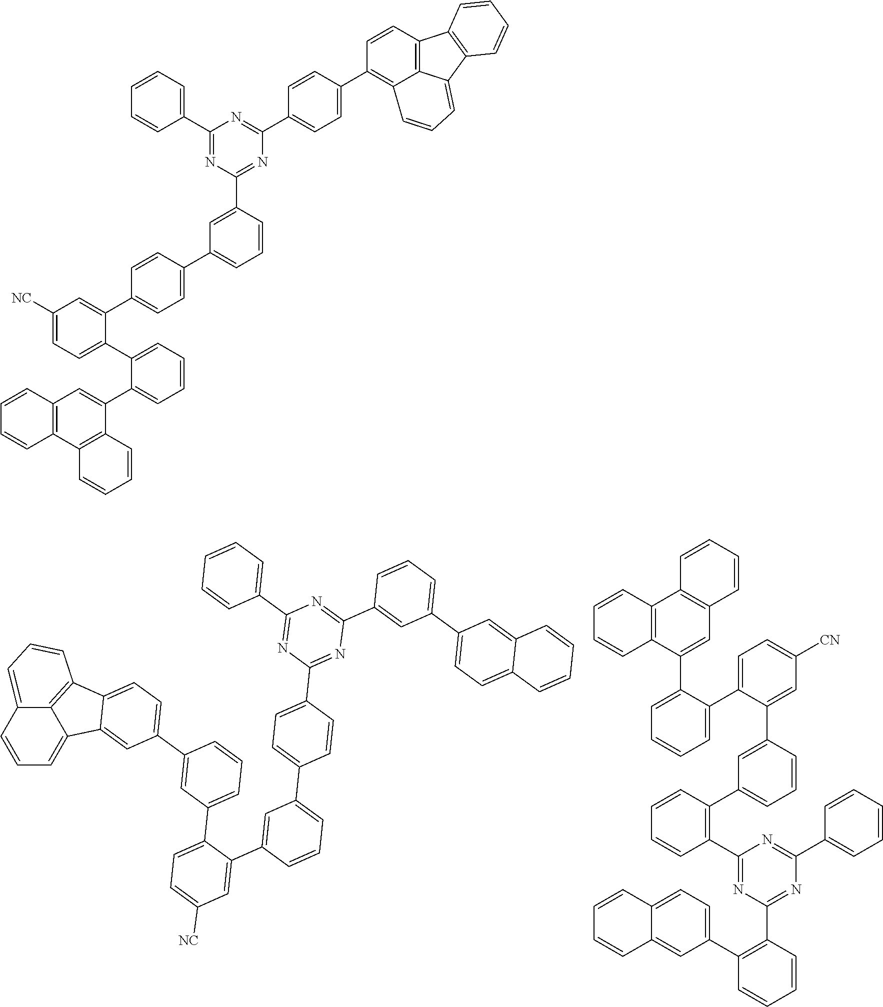

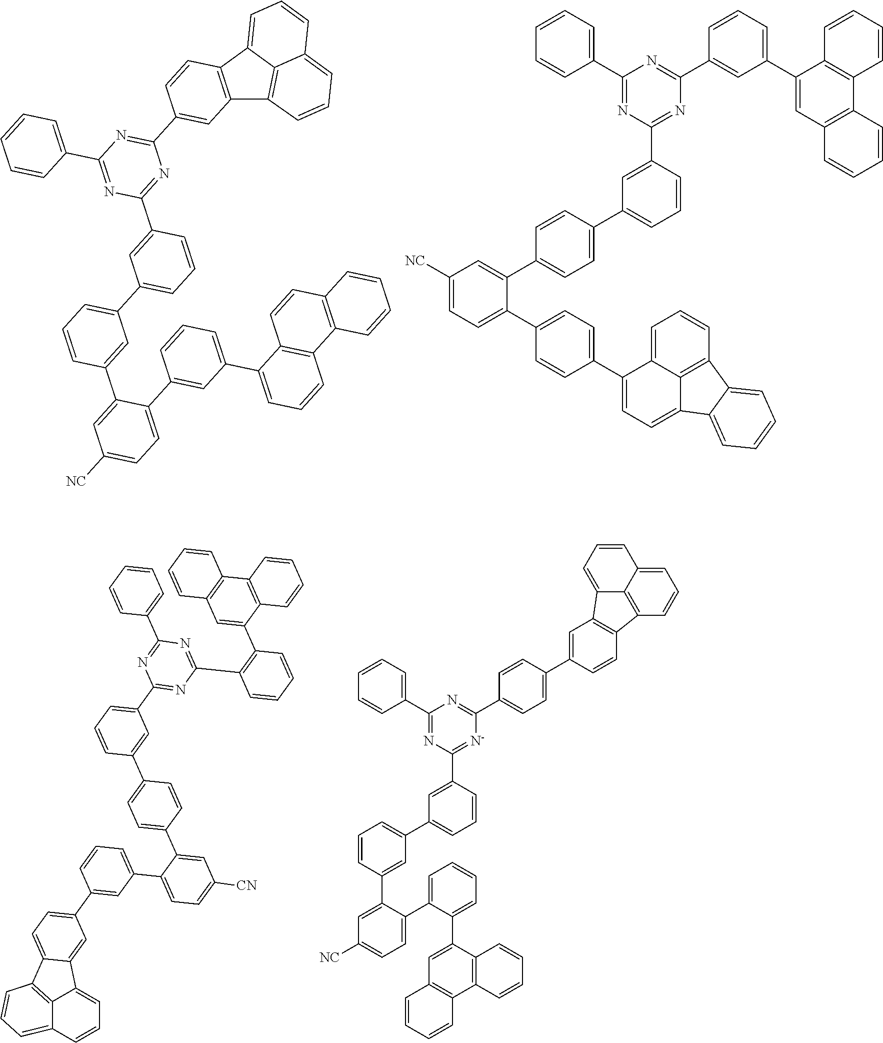

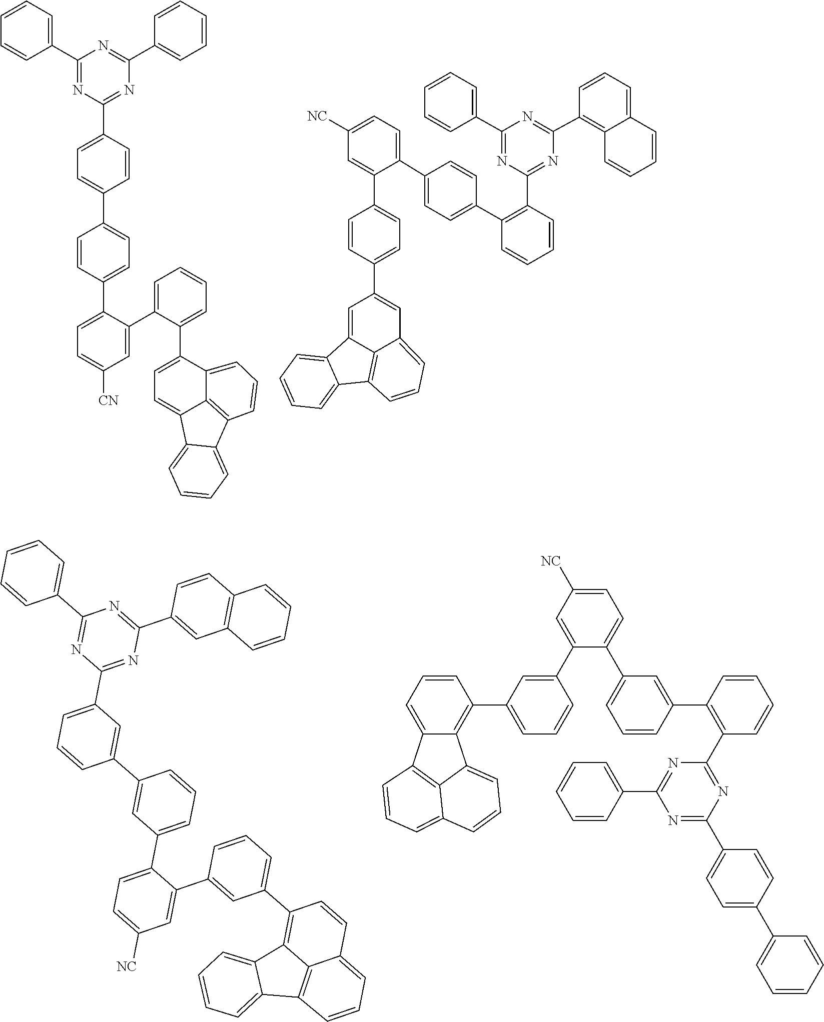

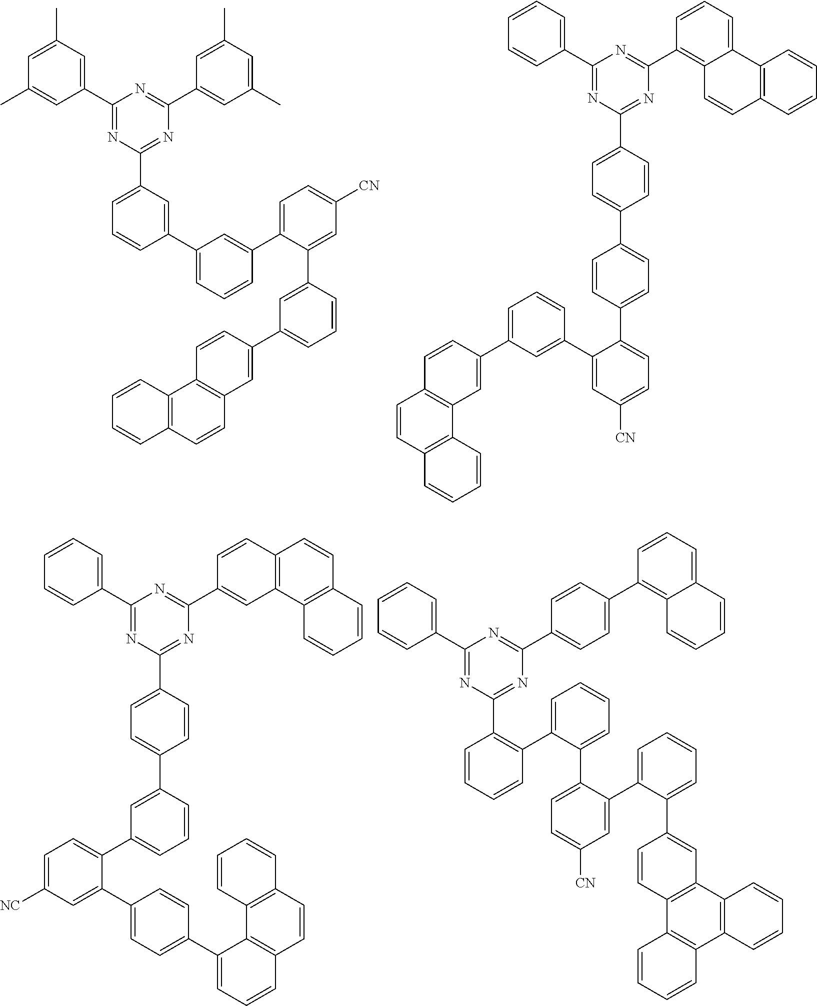

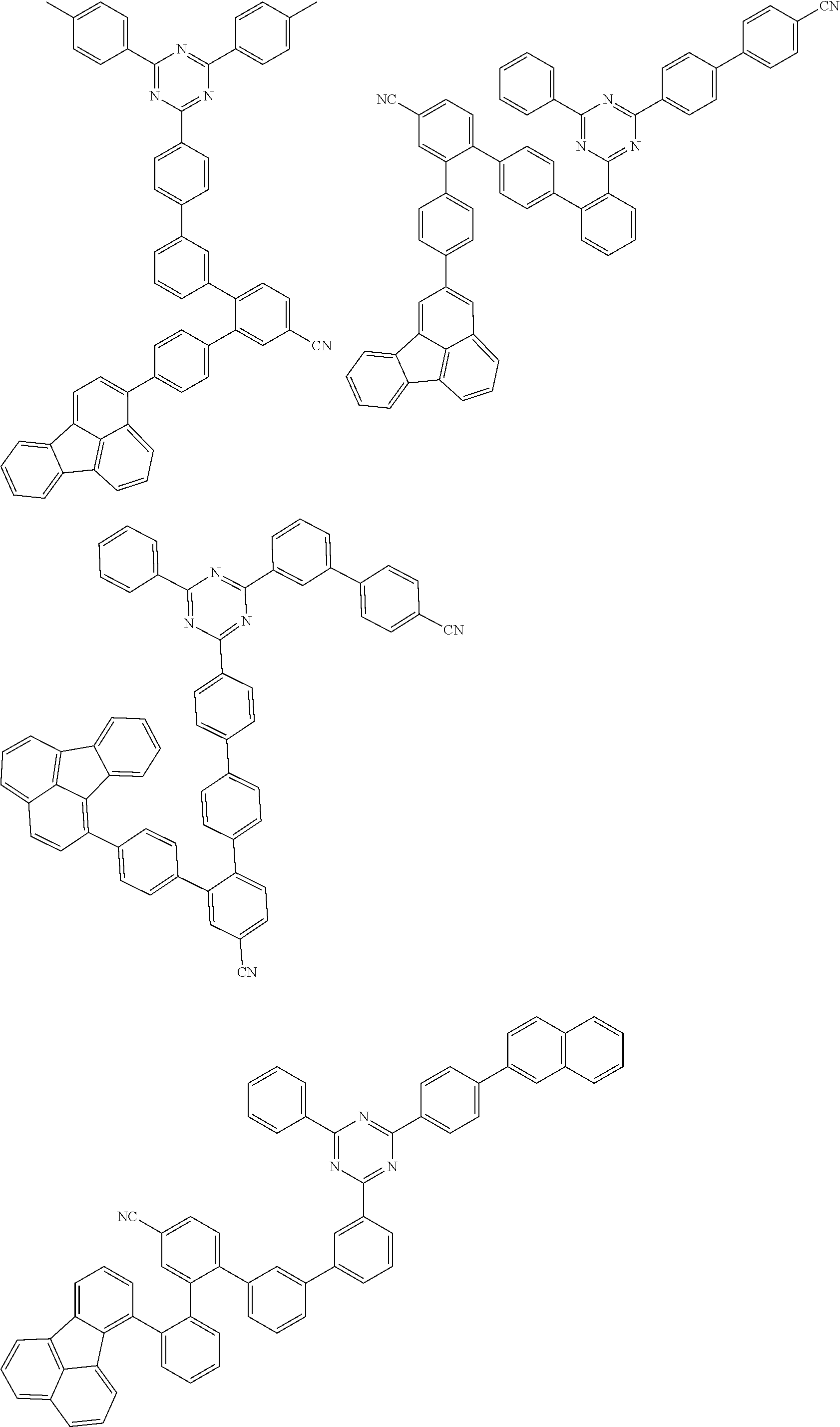

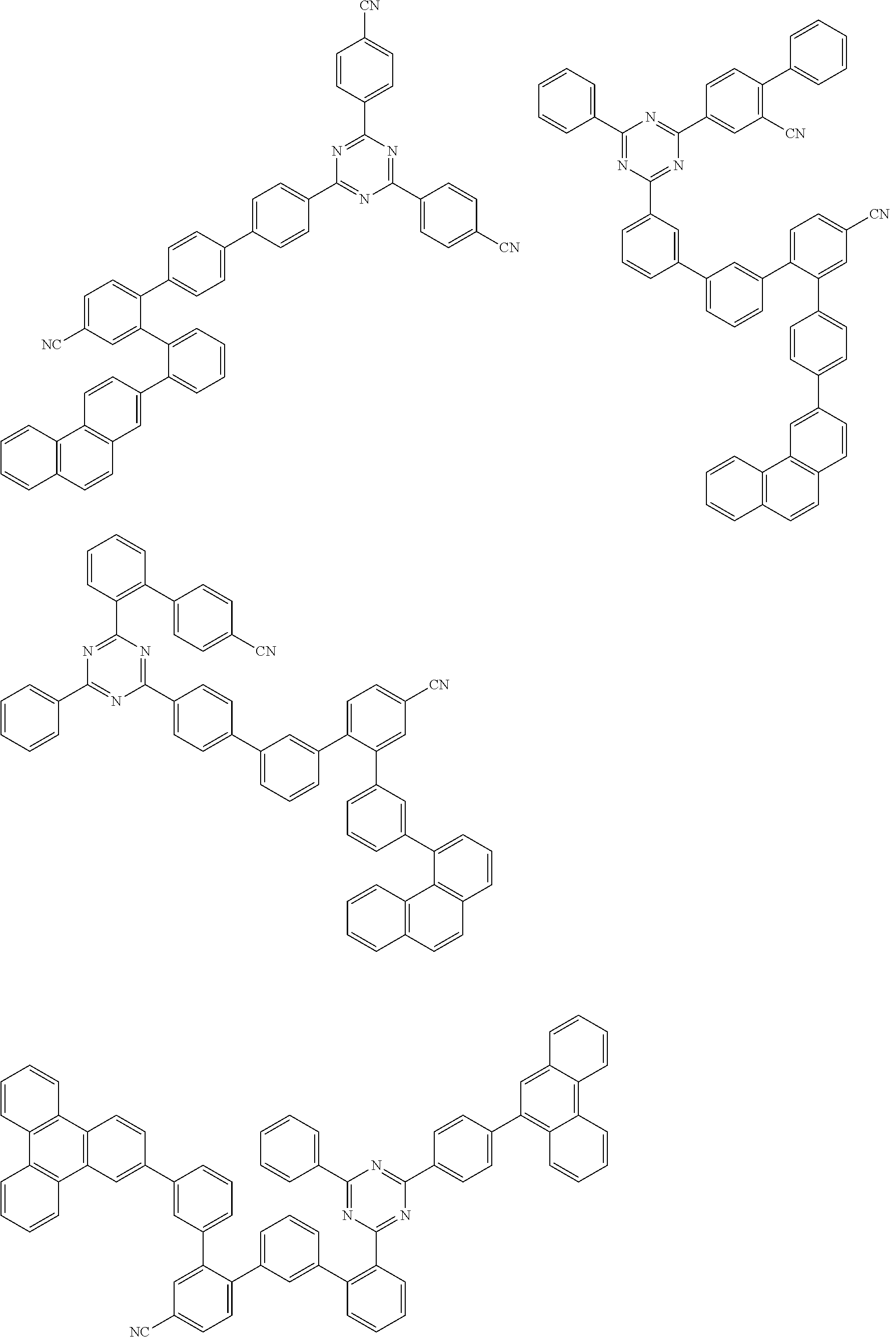 [SMILES: N#Cc1ccc(-c2nc(-c3ccc(C#N)cc3)nc(-c3ccc(-c4ccc(-c5ccc(C#N)cc5-c5ccccc5-c5ccc6c(ccc7ccccc76)c5)cc4)cc3)n2)cc1 ; N#Cc1ccc(-c2cccc(-c3cccc(-c4nc(-c5ccccc5)nc(-c5ccc(-c6ccccc6)c(C#N)c5)n4)c3)c2)c(-c2ccc(-c3ccc4ccc5ccccc5c4c3)cc2)c1 ; N#Cc1ccc(-c2ccccc2-c2nc(-c3ccccc3)nc(-c3ccc(-c4cccc(-c5ccc(C#N)cc5-c5cccc(-c6cccc7ccc8ccccc8c67)c5)c4)cc3)n2)cc1 ; N#Cc1ccc(-c2cccc(-c3ccccc3-c3nc(-c4ccccc4)nc(-c4ccc(-c5cc6ccccc6c6ccccc56)cc4)n3)c2)c(-c2cccc(-c3ccc4c5ccccc5c5ccccc5c4c3)c2)c1]

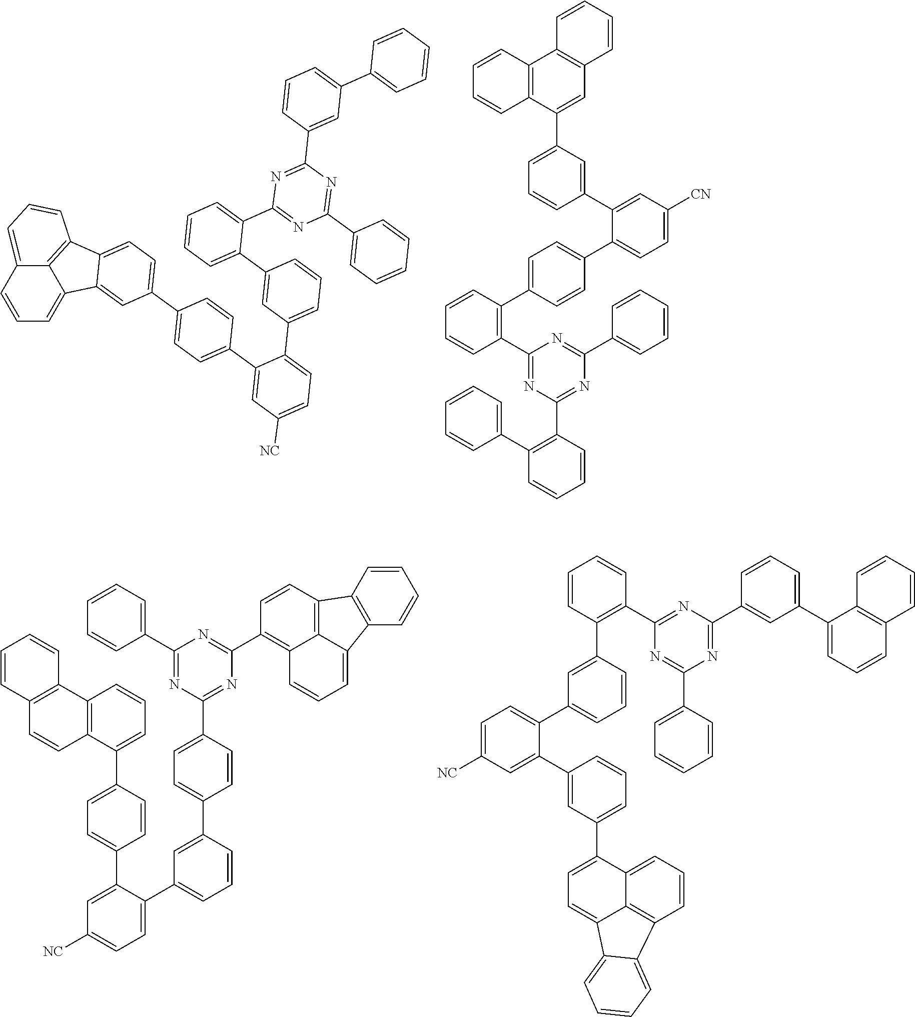 [SMILES: N#Cc1ccc(-c2cccc(-c3ccccc3-c3nc(-c4ccccc4)nc(-c4cccc(-c5ccccc5)c4)n3)c2)c(-c2ccc(-c3ccc4c(c3)-c3cccc5cccc-4c35)cc2)c1 ; N#Cc1ccc(-c2ccc(-c3ccccc3-c3nc(-c4ccccc4)nc(-c4ccccc4-c4ccccc4)n3)cc2)c(-c2cccc(-c3cc4ccccc4c4ccccc34)c2)c1 ; N#Cc1ccc(-c2cccc(-c3ccc(-c4nc(-c5ccccc5)nc(-c5ccc6c7c(cccc57)-c5ccccc5-6)n4)cc3)c2)c(-c2ccc(-c3cccc4c3ccc3ccccc34)cc2)c1 ; N#Cc1ccc(-c2cccc(-c3ccccc3-c3nc(-c4ccccc4)nc(-c4cccc(-c5cccc6ccccc56)c4)n3)c2)c(-c2cccc(-c3ccc4c5c(cccc35)-c3ccccc3-4)c2)c1]

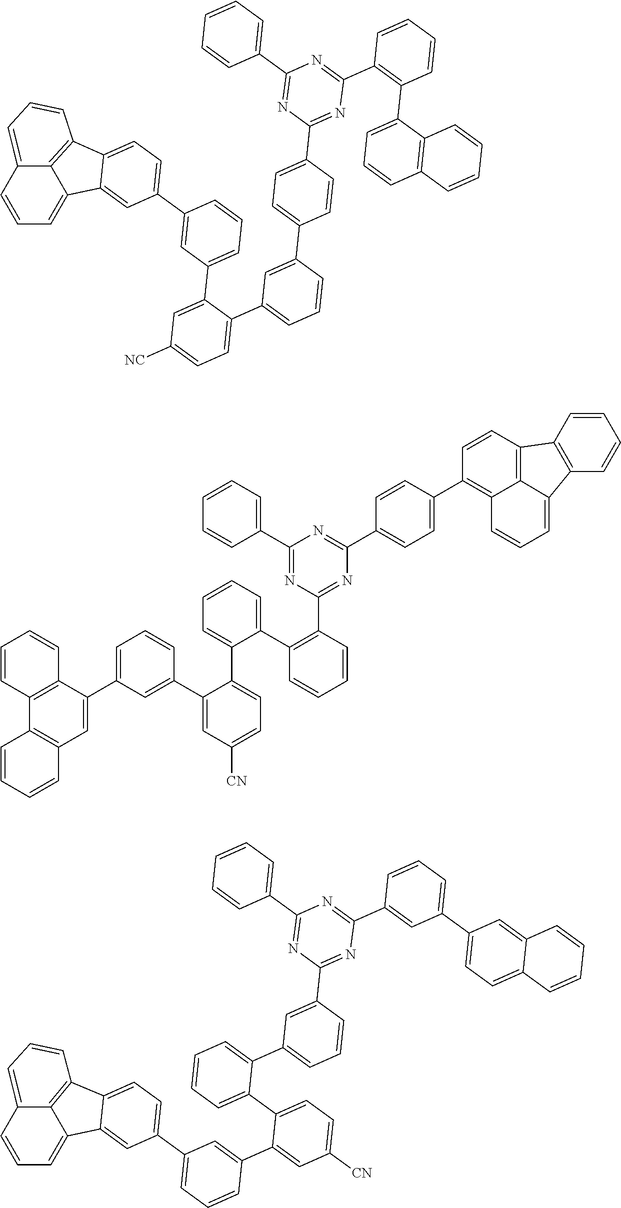 [SMILES: N#Cc1ccc(-c2cccc(-c3ccc(-c4nc(-c5ccccc5)nc(-c5ccccc5-c5cccc6ccccc56)n4)cc3)c2)c(-c2cccc(-c3ccc4c(c3)-c3cccc5cccc-4c35)c2)c1 ; N#Cc1ccc(-c2ccccc2-c2ccccc2-c2nc(-c3ccccc3)nc(-c3ccc(-c4ccc5c6c(cccc46)-c4ccccc4-5)cc3)n2)c(-c2cccc(-c3cc4ccccc4c4ccccc34)c2)c1 ; N#Cc1ccc(-c2ccccc2-c2cccc(-c3nc(-c4ccccc4)nc(-c4cccc(-c5ccc6ccccc6c5)c4)n3)c2)c(-c2cccc(-c3ccc4c(c3)-c3cccc5cccc-4c35)c2)c1]

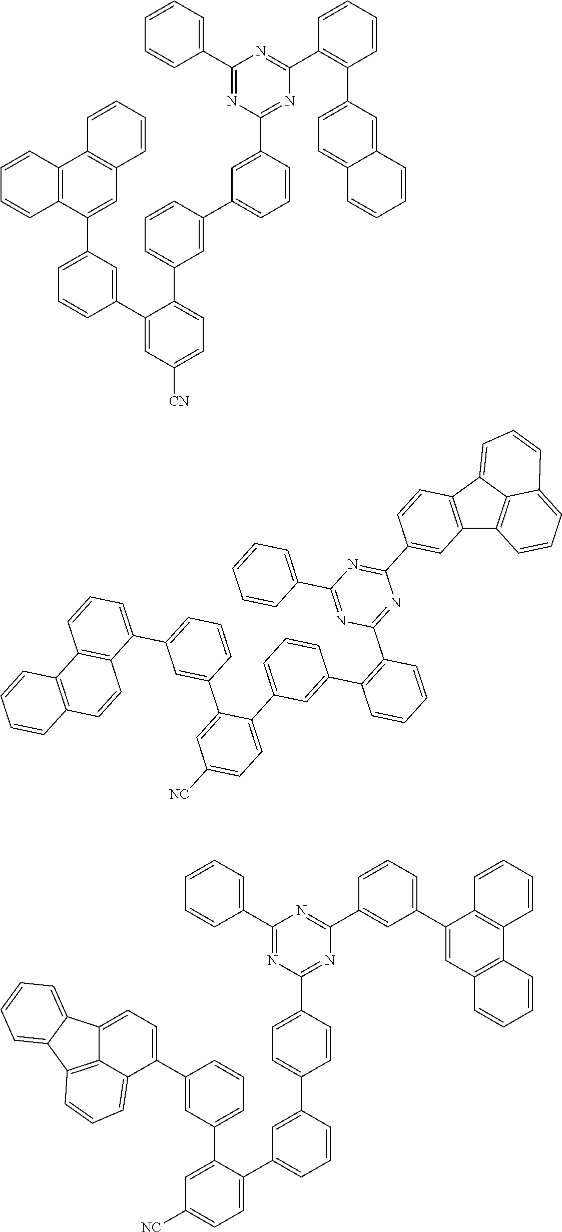 [SMILES: N#Cc1ccc(-c2cccc(-c3cccc(-c4nc(-c5ccccc5)nc(-c5ccccc5-c5ccc6ccccc6c5)n4)c3)c2)c(-c2cccc(-c3cc4ccccc4c4ccccc34)c2)c1 ; N#Cc1ccc(-c2cccc(-c3ccccc3-c3nc(-c4ccccc4)nc(-c4ccc5c(c4)-c4cccc6cccc-5c46)n3)c2)c(-c2cccc(-c3cccc4c3ccc3ccccc34)c2)c1 ; N#Cc1ccc(-c2cccc(-c3ccc(-c4nc(-c5ccccc5)nc(-c5cccc(-c6cc7ccccc7c7ccccc67)c5)n4)cc3)c2)c(-c2cccc(-c3ccc4c5c(cccc35)-c3ccccc3-4)c2)c1]

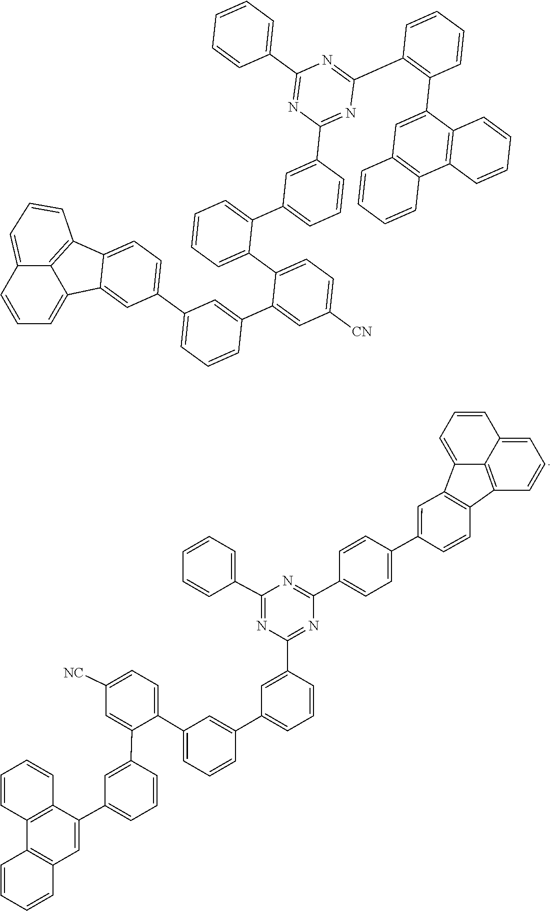 [SMILES: N#Cc1ccc(-c2ccccc2-c2cccc(-c3nc(-c4ccccc4)nc(-c4ccccc4-c4cc5ccccc5c5ccccc45)n3)c2)c(-c2cccc(-c3ccc4c(c3)-c3cccc5cccc-4c35)c2)c1 ; N#Cc1ccc(-c2cccc(-c3cccc(-c4nc(-c5ccccc5)nc(-c5ccc(-c6ccc7c(c6)-c6cccc8cccc-7c68)cc5)n4)c3)c2)c(-c2cccc(-c3cc4ccccc4c4ccccc34)c2)c1]

Meanwhile, the compound of Chemical Formula 1 can be prepared by a preparation method as shown in Reaction Scheme 1 below. The preparation method can be more specifically described in Synthesis Examples described below.

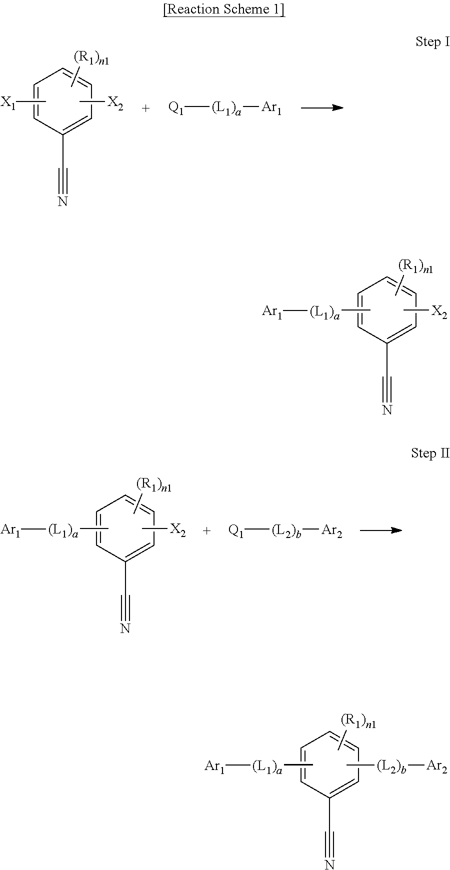

In Reaction Scheme 1, R₁, L₁, L₂, a, b, n1, n2, Ar₁, and Ar₂ are as defined in Chemical Formula 1, and

Q₁ and Q₂ are the same or different from each other, and each independently is BO₂C₂(CH₃)₄ or B(OH)₂, preferably B(OH)₂;

X₁ and X₂ are different from each other, and each independently is halogen, preferably Cl, Br, or I.

For example, X₁ can be Br or I, more preferably Br, and X₂ can be C₁ or Br, more preferably Cl.

Specifically, Reaction Scheme 1 comprises performing a first reaction (Step I) to introduce an aromatic fused polycyclic ring of carbon number 14 to 60 via an arylene linker at a specific position of a benzene ring substituted with a cyano group, followed by a second reaction (Step II) to introduce a heterocyclic ring containing at least one N via an additional arylene linker at another specific position of the benzene ring.

For example, in Reaction Scheme 1, a first reaction (Step I) can be first performed by reacting a compound comprising a benzene ring substituted with a cyano group and different halogen groups, X₁ and X₂, and a compound comprising Q₁, which is a pinacolborane group, BO₂C₂(CH₃)₄, or a boronic acid group, B(OH)₂, and an aromatic fused polycyclic ring of carbon number 14 to 60, which is bonded to Q₁ via an arylene linker, in the presence of a base and a Pd catalyst. In the first reaction (Step I), an aromatic fused polycyclic ring of carbon number 14 to 60 is introduced to the specific position where X₁ is bonded in the starting compound comprising a benzene ring substituted with a cyano group, via an arylene linker.

In Reaction Scheme 1, after performing the first reaction (Step I) to introduce the aromatic fused polycyclic ring, as described above, a second reaction (Step II) can be performed by reacting the resultant compound of the first reaction, comprising the remaining halogen group, X₂, bonded at another specific position of the benzene ring, and a compound comprising Q₂, which is a pinacolborane group, BO₂C₂(CH₃)₄, or a boronic acid group, B(OH)₂, and a heterocyclic ring containing at least one N, which is bonded to Q₂ via an arylene linker, in the presence of a base and a Pd catalyst. In the second reaction (Step II), a heterocyclic ring containing at least one N is introduced to the specific position where X₂ is bonded in the starting compound comprising a benzene ring substituted with a cyano group, via an arylene linker, after introducing an aromatic fused polycyclic ring of carbon number 14 to 60 to the specific position where X₁ is bonded in the starting compound comprising a benzene ring substituted with a cyano group, via an arylene linker.

Preferably, in Reaction Scheme 1, Q₁ and Q₂ can be B(OH)₂, X₁ can be Br, and X₂ can be Cl. In addition, in the preparation method according to the Reaction Scheme 1, detailed reaction conditions and processes can be applied as known in the art. The preparation method can be more specifically described in Synthesis Examples described below.

For example, potassium carbonate (K₂CO₃), sodium bicarbonate (NaHCO₃), cesium carbonate (Cs₂CO₃), sodium acetate (NaOAc), potassium acetate (KOAc), sodium tert-butoxide (NaOtBu), sodium ethoxide (NaOEt), triethylamine (Et₃N), N,N-diisopropylethylamine (EtN(iPr)₂), or the like can be used as the base. Preferably, the base can be potassium carbonate (K₂CO₃), cesium carbonate (Cs₂CO₃), potassium acetate (KOAc), sodium tert-butoxide (NaOtBu), or N,N-diisopropylethylamine (EtN(iPr)₂).

In addition, tetrakis(triphenylphosphine)palladium (0) (Pd(PPh₃)₄), tris(dibenzylideneacetone)dipalladium (0) (Pd₂(dba)₃), bis(tri-(tert-butyl)-phosphine)palladium (0) (Pd(P-tBus)₂), bis(dibenzylideneacetone)palladium (0) (Pd(dba)₂), or palladium(II) acetate (Pd(OAc)₂) can be used as the palladium catalyst. Preferably, the palladium catalyst in the first reaction (Step 1) of Reaction Scheme 1 can be tetrakis(triphenylphosphine)palladium (0) (Pd(PPh₃)₄). The palladium catalyst in the second reaction (Step II) of Reaction Scheme 1 can be palladium (II) acetate (Pd(OAc)₂).

Meanwhile, in the present disclosure, provided is an organic light emitting device including the above-mentioned compound of Chemical Formula 1. As an example, provided is an organic light emitting device including: a first electrode; a second electrode that is opposite to the first electrode; and one or more organic material layers that are between the first electrode and the second electrode, wherein at least one organic material layer of the one or more organic material layers includes the compound of Chemical Formula 1.

In the present disclosure, when a component is located “on” another component, this includes not only when a component is adjacent to another component, but also when there is another component between the two components.

In the present disclosure, when a part “includes or comprises” a component, it is meant to be inclusive of other components, not exclusive of other components, unless specifically stated to the contrary.

The organic material layer of the organic light emitting device of the present disclosure can have a single-layer structure, or a multilayered structure in which two or more organic material layers are stacked. For example, the organic light emitting device of the present disclosure can have a structure including a hole injection layer, a hole transport layer, an electron blocking layer, a light emitting layer, a hole blocking layer, an electron transport layer, an electron injection layer and the like as the organic material layer. However, the structure of the organic light emitting device is not limited thereto, and it can include a smaller number of organic layers.

In addition, the organic material layer can include a hole injection layer, a hole transport layer, or a layer that simultaneously injects and transports holes, and the hole injection layer, the hole transport layer, or the layer that simultaneously injects and transports holes includes the compound of Chemical Formula 1.

For example, the hole transport layer can have a first hole transport layer and a second hole transport layer, and the first hole transport layer or the second hole transport layer includes the compound of Chemical Formula 1.

In addition, the organic material layer can include an electron blocking layer, and the electron blocking layer includes the compound of Chemical Formula 1.

In addition, the organic material layer can include a light emitting layer, and the light emitting layer includes the compound of Chemical Formula 1.

In addition, the organic material layer can include a hole blocking layer, and the hole blocking layer includes the compound of Chemical Formula 1.

In addition, the organic material layer can include a light emitting layer and a hole blocking layer, and the hole blocking layer includes the compound of Chemical Formula 1.

In addition, the organic material layer can include an electron transport layer or an electron injection layer, or the layer that simultaneously transports and injects electrons. The electron transport layer, the electron injection layer, or the layer that simultaneously transports and injects electrons includes the compound of Chemical Formula 1.

In addition, the organic material layer can include a light emitting layer and an electron transport layer, and the electron transport layer can include a compound of Chemical Formula 1.

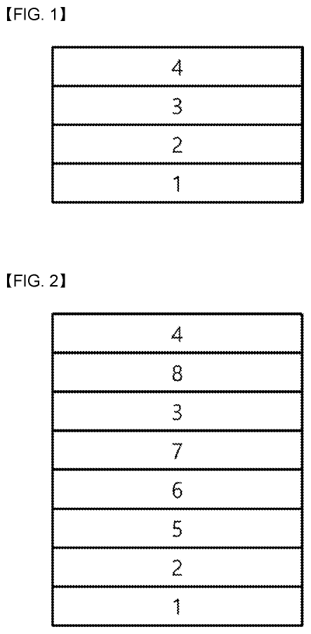

Meanwhile, the organic light emitting device according to the present disclosure can be a normal type organic light emitting device in which an anode, one or more organic material layers and a cathode are sequentially stacked on a substrate. Further, the organic light emitting device according to the present disclosure can be an inverted type organic light emitting device in which a cathode, one or more organic material layers and an anode are sequentially stacked on a substrate. For example, the structure of an organic light emitting device according to an embodiment of the present disclosure is illustrated in FIGS. 1 and 2.

FIG. 1 shows an example of an organic light emitting device including a substrate 1, an anode 2, a light emitting layer 3, and a cathode 4. In this structure, the compound of Chemical Formula 1 can be included in the light emitting layer.

FIG. 2 shows an example of an organic light emitting device including a substrate 1, an anode 2, a hole injection layer 5, a first hole transport layer 6, a second hole transport layer 7, a light emitting layer 3, an electron injection and transport layer 8, and a cathode 4. In this structure, the compound of Chemical Formula 1 can be included in one or more layers of the hole injection layer, the first hole transport layer, the second hole transport layer, the light emitting layer, and the electron injection and transport layer. Specifically, the compound of Chemical Formula 1 can be included in the electron injection and transport layer.

In addition, the organic light emitting device can further include a hole blocking layer, and the hole blocking layer includes the compound of Chemical Formula 1.

For example, the compound of Chemical Formula 1 can be included in the hole blocking layer, or the electron transport layer, or the electron injection and transport layer.

The organic light emitting device according to the present disclosure can be manufactured by materials and methods known in the art, except that one or more layers of the organic material layers include the compound of Chemical Formula 1. Moreover, when the organic light emitting device includes a plurality of organic material layers, the organic material layers can be formed of the same material or different materials.

For example, the organic light emitting device according to the present disclosure can be manufactured by sequentially stacking a first electrode, an organic material layer and a second electrode on a substrate. In this case, the organic light emitting device can be manufactured by depositing a metal, metal oxides having conductivity, or an alloy thereof on the substrate using a PVD (physical vapor deposition) method such as a sputtering method or an e-beam evaporation method to form an anode, forming organic material layers including the hole injection layer, the hole transport layer, the light emitting layer and the electron transport layer thereon, and then depositing a material that can be used as the cathode thereon. In addition to such a method, the organic light emitting device can be manufactured by sequentially depositing a cathode material, an organic material layer and an anode material on a substrate.

Further, the compound of Chemical Formula 1 can be formed into an organic material layer by a solution coating method as well as a vacuum deposition method at the time of manufacturing an organic light emitting device. In particular, the compound of Chemical Formula 1 has excellent solubility in a solvent used for the solution coating method, and thus it is easy to apply the solution coating method. Herein, the solution coating method means a spin coating, a dip coating, a doctor blading, an inkjet printing, a screen printing, a spray method, a roll coating, or the like, but is not limited thereto.

Accordingly, in the present disclosure, there is provided a coating composition including the compound of Chemical Formula 1 and a solvent.

The solvent is not particularly limited as long as it is a solvent capable of dissolving or dispersing the compound according to the present disclosure. Examples thereof can include a chlorine-based solvent such as chloroform, methylene chloride, 1,2-dichloroethane, 1,1,2-trichloroethane, chlorobenzene and o-dichlorobenzene; an ether-based solvent such as tetrahydrofuran and dioxane; an aromatic hydrocarbon-based solvent such as toluene, xylene, trimethylbenzene, and mesitylene; an aliphatic hydrocarbon-based solvent such as cyclohexane, methylcyclohexane, n-pentane, n-hexane, n-heptane, n-octane, n-nonane, and n-decane; a ketone-based solvent such as acetone, methyl ethyl ketone, and cyclohexanone; an ester-based solvent such as ethyl acetate, butyl acetate, and ethyl cellosolve acetate; a polyhydric alcohol and derivatives thereof such as ethylene glycol, ethylene glycol monobutyl ether, ethylene glycol monoethyl ether, ethylene glycol monomethyl ether, dimethoxyethane, propylene glycol, diethoxymethane, triethylene glycol monoethyl ether, glycerin, and 1,2-hexanediol; an alcohol-based solvent such as methanol, ethanol, propanol, isopropanol and cyclohexanol; a sulfoxide-based solvent such as dimethyl sulfoxide; and an amide-based solvent such as N-methyl-2-pyrrolidone and N,N-dimethylformamide; a benzoate-based solvent such as butyl benzoate and methyl 2-methoxy benzoate; tetralin; 3-phenoxy-toluene and the like. In addition, the above-mentioned solvents can be used alone or in mixture of two or more.

In addition, a viscosity of the coating composition is preferably 1 cP to 10 cP, and coating is easy within the above range. In addition, a concentration of the compound according to the present disclosure in the coating composition is preferably 0.1 wt/v % to 20 wt/v %.

In addition, also provided is a method for forming a functional layer using the above-described coating composition. Specifically, it includes the steps of coating the coating composition according to the present disclosure in a solution process; and heat-treating the coated coating composition.

The heat-treatment in the heat-treatment step is preferably performed at 150° C. to 230° C. In addition, the heat-treatment is performed for 1 minute to 3 hours, more preferably for 10 minutes to 1 hour. In addition, the heat-treatment is preferably performed under an inert gas atmosphere such as argon or nitrogen.

For example, the first electrode is an anode, and the second electrode is a cathode, or alternatively, the first electrode is a cathode and the second electrode is an anode.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides such as ZnO:Al or SnO₂:Sb; conductive polymers such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

The hole injection layer is a layer for injecting holes from the electrode, and the hole injection material is preferably a compound which has a capability of transporting the holes, thus has a hole injecting effect in the anode and an excellent hole-injecting effect to the light emitting layer or the light emitting material, prevents excitons produced in the light emitting layer from moving to an electron injection layer or the electron injection material, and is excellent in the ability to form a thin film. It is preferable that a HOMO (highest occupied molecular orbital) of the hole injection material is between the work function of the anode material and a HOMO of a peripheral organic material layer. Specific examples of the hole injection material include metal porphyrin, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

The hole transport layer is a layer that receives holes from a hole injection layer and transports the holes to the light emitting layer. The hole transport material is suitably a material having large mobility to the holes, which can receive holes from the anode or the hole injection layer and transfer the holes to the light emitting layer. Specific examples thereof include an arylamine-based organic material, a conductive polymer, a block copolymer in which a conjugate portion and a non-conjugate portion are present together, and the like, but are not limited thereto.

The light emitting material is suitably a material capable of emitting light in a visible ray region by receiving holes and electrons from the hole transport layer and the electron transport layer, respectively, to combine them, and having good quantum efficiency to fluorescence or phosphorescence. Specific examples thereof include 8-hydroxy-quinoline aluminum complex (Alq₃); a carbazole-based compound; a dimerized styryl compound; BAlq; a 10-hydroxybenzo quinoline-metal compound; a benzoxazole-, benzothiazole- and benzimidazole-based compound; a poly(p-phenylenevinylene) (PPV)-based polymer; a spiro compound; polyfluorene, rubrene, and the like, but are not limited thereto.

The light emitting layer can include a host material and a dopant material. The host material can be a fused aromatic ring derivative, a heterocycle-containing compound or the like. Specific examples of the fused aromatic ring derivative include anthracene derivatives, pyrene derivatives, naphthalene derivatives, pentacene derivatives, phenanthrene compounds, fluoranthene compounds, and the like. Examples of the heterocycle containing compound include carbazole derivatives, dibenzofuran derivatives, ladder-type furan compounds, pyrimidine derivatives, and the like, but are not limited thereto.

The dopant material includes an aromatic amine derivative, a styrylamine compound, a boron complex, a fluoranthene compound, a metal complex, and the like. Specifically, the aromatic amine derivative is a substituted or unsubstituted fused aromatic ring derivative having an arylamino group, and examples thereof include pyrene, anthracene, chrysene, periflanthene and the like, which have an arylamino group. The styrylamine compound is a compound where at least one arylvinyl group is substituted in substituted or unsubstituted arylamine, in which one or two or more substituent groups selected from the group consisting of an aryl group, a silyl group, an alkyl group, a cycloalkyl group, and an arylamino group are substituted or unsubstituted. Specific examples thereof include styrylamine, styryldiamine, styryltriamine, styryltetramine, and the like, but are not limited thereto. Further, the metal complex includes an iridium complex, a platinum complex, and the like, but is not limited thereto. Preferably, the iridium complex is used as the dopant material.

The electron transport layer is a layer which receives electrons from an electron injection layer and transports the electrons to a light emitting layer, and an electron transport material is suitably a material which can receive electrons well from a cathode and transfer the electrons to a light emitting layer and has large mobility for electrons. Specific examples thereof include an Al complex of 8-hydroxyquinoline; a complex including Alq₃ (tris(8-hydroxyquinolino)aluminum); an organic radical compound; a hydroxyflavone-metal complex, and the like, but are not limited thereto. The electron transport layer can be used with any desired cathode material, as used according to the related art. In particular, appropriate examples of the cathode material include typical materials which have a low work function, followed by an aluminum layer or a silver layer. Specific examples thereof include cesium, barium, calcium, ytterbium, and samarium, in each case followed by an aluminum layer or a silver layer. Preferably, the compound according to the present disclosure is used as the organic material of the electron transport layer.

When the compound according to the present disclosure is used as the organic material of the electron transport layer, the compound can receive electrons from the cathode well and efficiently deliver electrons to the light emitting layer. Thus, it is possible to improve characteristics of the organic light emitting device, that is, the characteristics of low voltage, high efficiency, and long lifespan of the organic light emitting device can be further improved.

The electron injection layer is a layer which injects electrons from an electrode, and is preferably a compound which has a capability of transporting electrons, has an effect of injecting electrons from a cathode and an excellent effect of injecting electrons into a light emitting layer or a light emitting material, prevents excitons produced from the light emitting layer from moving to a hole injection layer, and is also excellent in the ability to form a thin film. Specific examples thereof include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

Examples of the metal complex compound include 8-hydroxy-quinolinato lithium, bis(8-hydroxyquinolinato)zinc, bis(8-hydroxyquinolinato)-copper, bis(8-hydroxyquinolinato)manganese, tris(8-hydroxyquinolinato)-aluminum, tris(2-methyl-8-hydroxyquinolinato)aluminum, tris(8-hydroxy-quinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)-(1-naphtholato)aluminum, bis(2-methyl-8-quinolinato)(2-naphtholato)gallium, and the like, but are not limited thereto.

The organic light emitting device according to the present disclosure can be a bottom emission device, a top emission device, or a double-sided light emitting device, and particularly, can be a bottom emission device that requires relatively high luminous efficiency.

In addition, the compound according to the present disclosure can be included in an organic solar cell or an organic transistor in addition to an organic light emitting device.

The preparation of the compound of Chemical Formula 1 and the organic light emitting device containing the same will be described in detail in the following examples. However, these examples are presented for illustrative purposes only, and are not intended to limit the scope of the present disclosure.

### SYNTHESIS EXAMPLES

### Synthesis Example 1. Synthesis of Compound 1

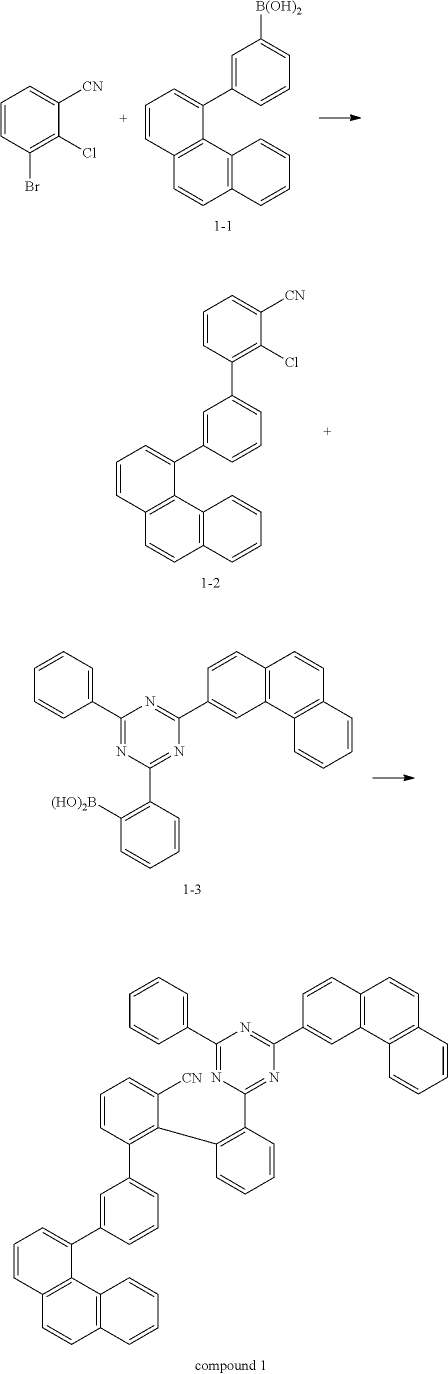 [SMILES: N#Cc1cccc(Br)c1Cl ; OB(O)c1cccc(-c2cccc3ccc4ccccc4c23)c1 ; N#Cc1cccc(-c2cccc(-c3cccc4ccc5ccccc5c34)c2)c1Cl ; OB(O)c1ccccc1-c1nc(-c2ccccc2)nc(-c2ccc3ccc4ccccc4c3c2)n1 ; N#Cc1cccc(-c2cccc(-c3cccc4ccc5ccccc5c34)c2)c1-c1ccccc1-c1nc(-c2ccccc2)nc(-c2ccc3ccc4ccccc4c3c2)n1]

3-bromo-2-chlorobenzonitrile (6.49 g, 30 mmol) and Compound 1-1 (9.84 g, 33 mmol) were added to tetrahydrofuran (THF, 300 mL). 2 M aqueous solution of potassium carbonate (K₂CO₃, 200 mL) and tetrakis(triphenylphosphine)palladium (0) (Pd(PPH₃)₄, 0.3 g) were added thereto. Then, the mixture was sufficiently stirred and refluxed for 5 hours. After cooling to room temperature and filtering, the resultant solid was recrystallized twice with toluene to produce Compound 1-2.

Compound 1-2 (11.70 g, 30 mmol) and compound 1-3 (14.96 g, 33 mmol) were added to tetrahydrofuran (THF, 300 mL). After adding 2 M aqueous solution of potassium carbonate (K₂CO₃, 200 mL), palladium (II) acetate (Pd(OAc)₂, 0.14 g), and 2-dicyclohexylphosphino-2′,6′-dimethoxybiphenyl (s-phos ligand, 0.50 g), and then it was stirred and refluxed for 5 hours. After cooling to room temperature and filtering, the resultant solid was recrystallized twice with toluene to produce Compound 1 (16.5 g, yield 72%).

MS: [M+H]⁺=763

### Synthesis Example 2. Synthesis of Compound 2

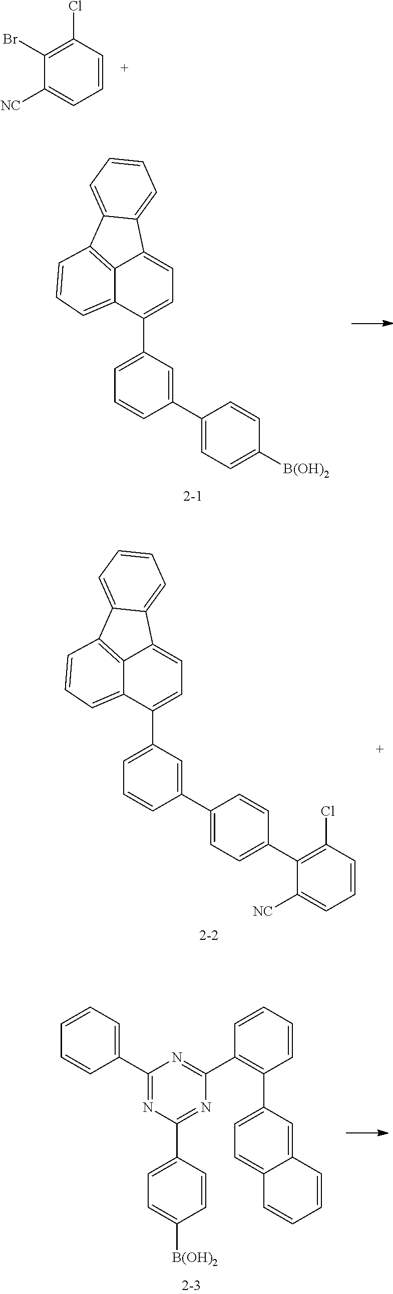 [SMILES: N#Cc1cccc(Cl)c1Br ; OB(O)c1ccc(-c2cccc(-c3ccc4c5c(cccc35)-c3ccccc3-4)c2)cc1 ; N#Cc1cccc(Cl)c1-c1ccc(-c2cccc(-c3ccc4c5c(cccc35)-c3ccccc3-4)c2)cc1 ; OB(O)c1ccc(-c2nc(-c3ccccc3)nc(-c3ccccc3-c3ccc4ccccc4c3)n2)cc1]

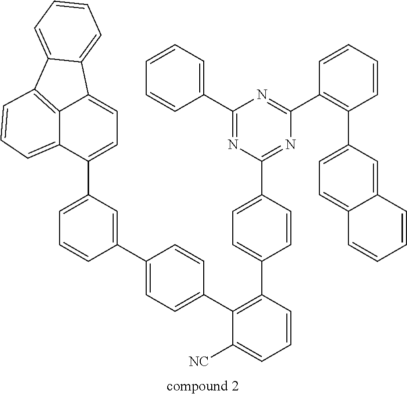 [SMILES: N#Cc1cccc(-c2ccc(-c3nc(-c4ccccc4)nc(-c4ccccc4-c4ccc5ccccc5c4)n3)cc2)c1-c1ccc(-c2cccc(-c3ccc4c5c(cccc35)-c3ccccc3-4)c2)cc1]

As shown in the above reaction scheme, after preparing Compound 2-2 using Compound 2-1 instead of Compound 1-1, Compound 2 was prepared in the same manner as in the preparation method of Synthesis Example 1, except for using Compound 2-3 instead of Compound 1-3.

MS: [M+H]⁺=889.

### Synthesis Example 3. Synthesis of Compound 3

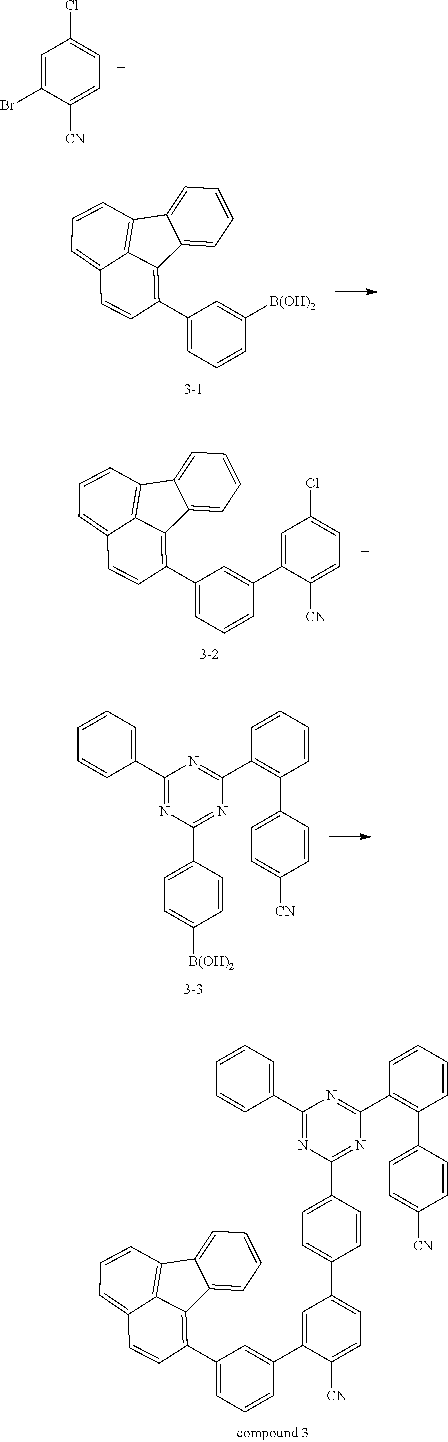 [SMILES: N#Cc1ccc(Cl)cc1Br ; OB(O)c1cccc(-c2ccc3cccc4c3c2-c2ccccc2-4)c1 ; N#Cc1ccc(Cl)cc1-c1cccc(-c2ccc3cccc4c3c2-c2ccccc2-4)c1 ; N#Cc1ccc(-c2ccccc2-c2nc(-c3ccccc3)nc(-c3ccc(B(O)O)cc3)n2)cc1 ; N#Cc1ccc(-c2ccccc2-c2nc(-c3ccccc3)nc(-c3ccc(-c4ccc(C#N)c(-c5cccc(-c6ccc7cccc8c7c6-c6ccccc6-8)c5)c4)cc3)n2)cc1]

As shown in the above reaction scheme, after preparing Compound 3-2 using Compound 3-1 instead of Compound 1-1, Compound 3 was prepared in the same manner as in the preparation method of Synthesis Example 1, except for using Compound 3-3 instead of Compound 1-3.

MS: [M+H]⁺=788.

### Synthesis Example 4. Synthesis of Compound 4

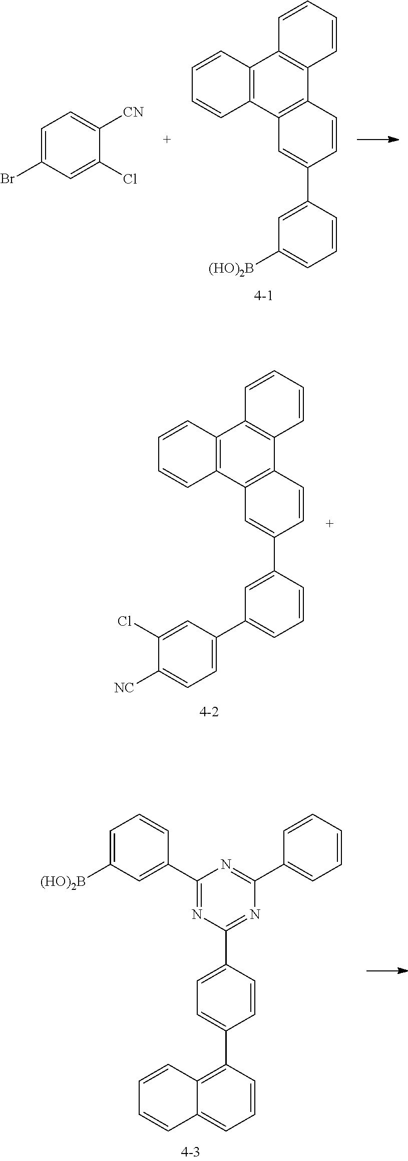 [SMILES: N#Cc1ccc(Br)cc1Cl ; OB(O)c1cccc(-c2ccc3c4ccccc4c4ccccc4c3c2)c1 ; N#Cc1ccc(-c2cccc(-c3ccc4c5ccccc5c5ccccc5c4c3)c2)cc1Cl ; OB(O)c1cccc(-c2nc(-c3ccccc3)nc(-c3ccc(-c4cccc5ccccc45)cc3)n2)c1]

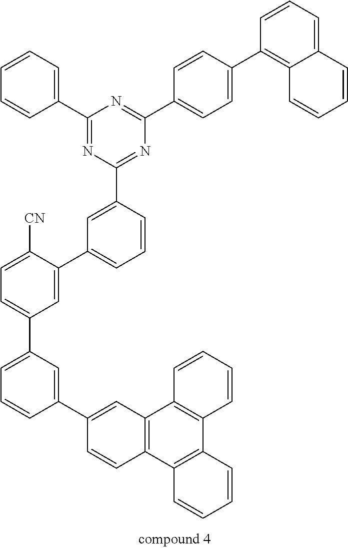 [SMILES: N#Cc1ccc(-c2cccc(-c3ccc4c5ccccc5c5ccccc5c4c3)c2)cc1-c1cccc(-c2nc(-c3ccccc3)nc(-c3ccc(-c4cccc5ccccc45)cc3)n2)c1]

As shown in the above reaction scheme, after preparing Compound 4-2 using Compound 4-1 instead of Compound 1-1, Compound 4 was prepared in the same manner as in Synthesis Example 1, except for using Compound 4-3 instead of Compound 1-3.

MS: [M+H]⁺=839.

### Synthesis Example 5. Synthesis of Compound 5

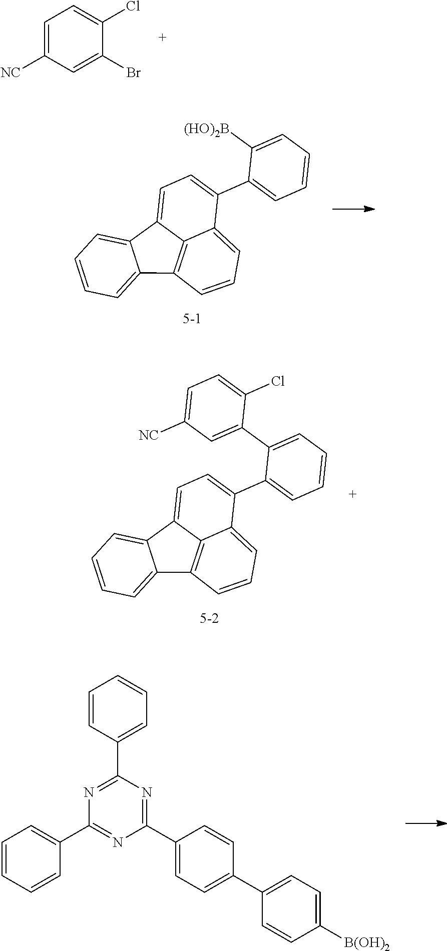 [SMILES: N#Cc1ccc(Cl)c(Br)c1 ; OB(O)c1ccccc1-c1ccc2c3c(cccc13)-c1ccccc1-2 ; N#Cc1ccc(Cl)c(-c2ccccc2-c2ccc3c4c(cccc24)-c2ccccc2-3)c1 ; OB(O)c1ccc(-c2ccc(-c3nc(-c4ccccc4)nc(-c4ccccc4)n3)cc2)cc1]

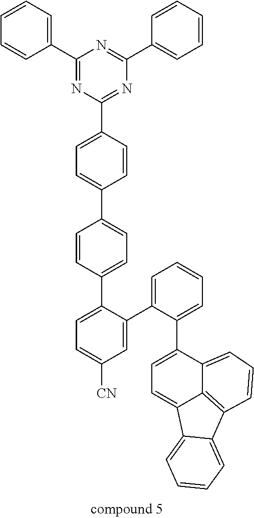 [SMILES: N#Cc1ccc(-c2ccc(-c3ccc(-c4nc(-c5ccccc5)nc(-c5ccccc5)n4)cc3)cc2)c(-c2ccccc2-c2ccc3c4c(cccc24)-c2ccccc2-3)c1]

As shown in the above reaction scheme, after preparing Compound 5-2 using Compound 5-1 instead of Compound 1-1, Compound 5 was prepared in the same manner as in Synthesis Example 1, except for using Compound 5-3 instead of Compound 1-3.

MS: [M+H]⁺=763.

### Synthesis Example 6. Synthesis of Compound 6

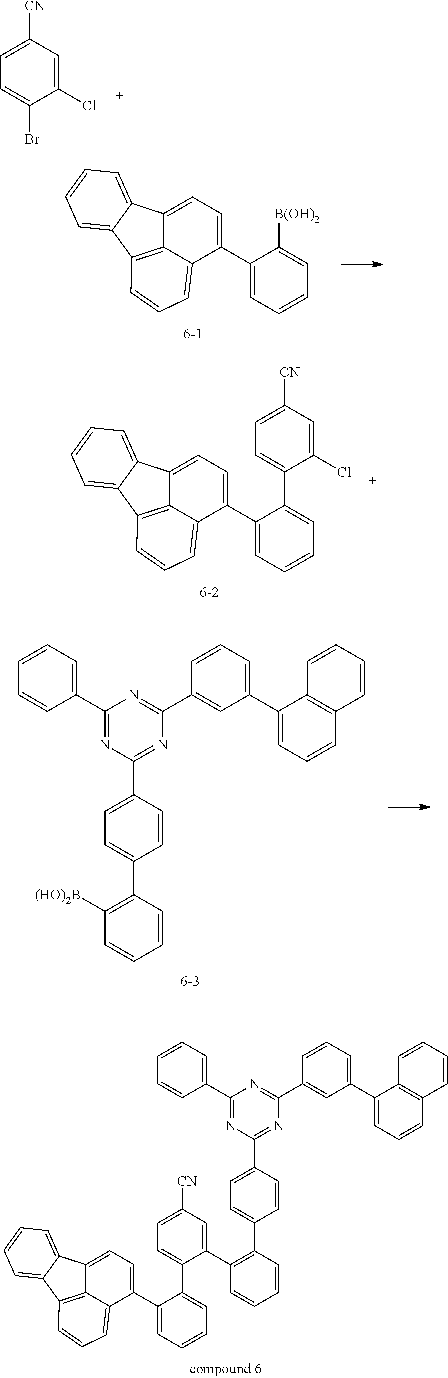 [SMILES: N#Cc1ccc(Br)c(Cl)c1 ; OB(O)c1ccccc1-c1ccc2c3c(cccc13)-c1ccccc1-2 ; N#Cc1ccc(-c2ccccc2-c2ccc3c4c(cccc24)-c2ccccc2-3)c(Cl)c1 ; OB(O)c1ccccc1-c1ccc(-c2nc(-c3ccccc3)nc(-c3cccc(-c4cccc5ccccc45)c3)n2)cc1 ; N#Cc1ccc(-c2ccccc2-c2ccc3c4c(cccc24)-c2ccccc2-3)c(-c2ccccc2-c2ccc(-c3nc(-c4ccccc4)nc(-c4cccc(-c5cccc6ccccc56)c4)n3)cc2)c1]

As shown in the above reaction scheme, after preparing Compound 6-2 using Compound 6-1 instead of Compound 1-1, Compound 6 was prepared in the same manner as in the preparation method of Synthesis Example 1, except for using Compound 6-3 instead of Compound 1-3.

MS: [M+H]⁺=889.

### Synthesis Example 7. Synthesis of Compound 7

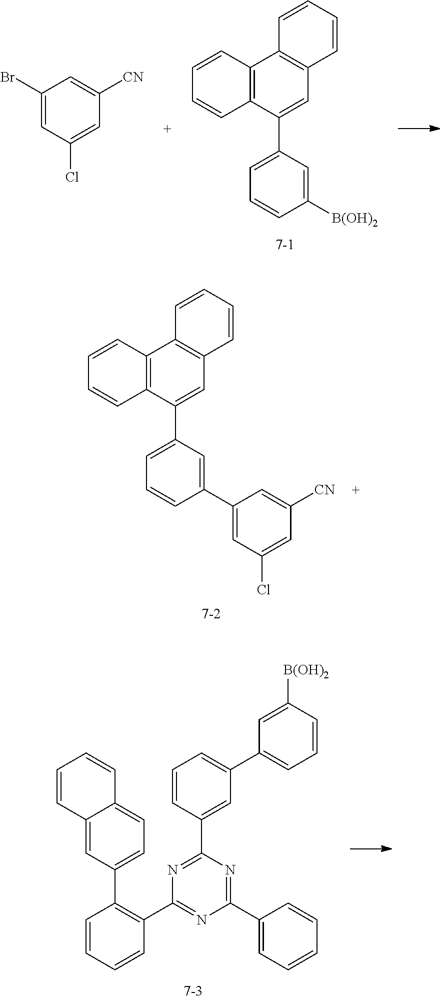 [SMILES: N#Cc1cc(Cl)cc(Br)c1 ; OB(O)c1cccc(-c2cc3ccccc3c3ccccc23)c1 ; N#Cc1cc(Cl)cc(-c2cccc(-c3cc4ccccc4c4ccccc34)c2)c1 ; OB(O)c1cccc(-c2cccc(-c3nc(-c4ccccc4)nc(-c4ccccc4-c4ccc5ccccc5c4)n3)c2)c1]

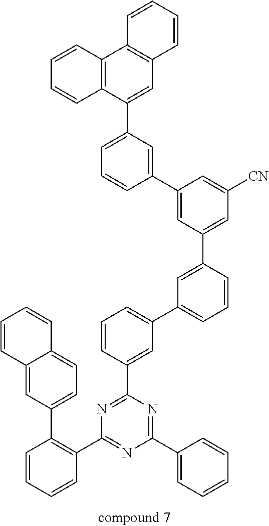 [SMILES: N#Cc1cc(-c2cccc(-c3cccc(-c4nc(-c5ccccc5)nc(-c5ccccc5-c5ccc6ccccc6c5)n4)c3)c2)cc(-c2cccc(-c3cc4ccccc4c4ccccc34)c2)c1]

As shown in the above reaction scheme, after preparing Compound 7-2 using Compound 7-1 instead of Compound 1-1, Compound 7 was prepared in the same manner as in Synthesis Example 1, except for using Compound 7-3 instead of Compound 1-3.

MS: [M+H]⁺=865.

### Synthesis Example 8. Synthesis of Compound 8

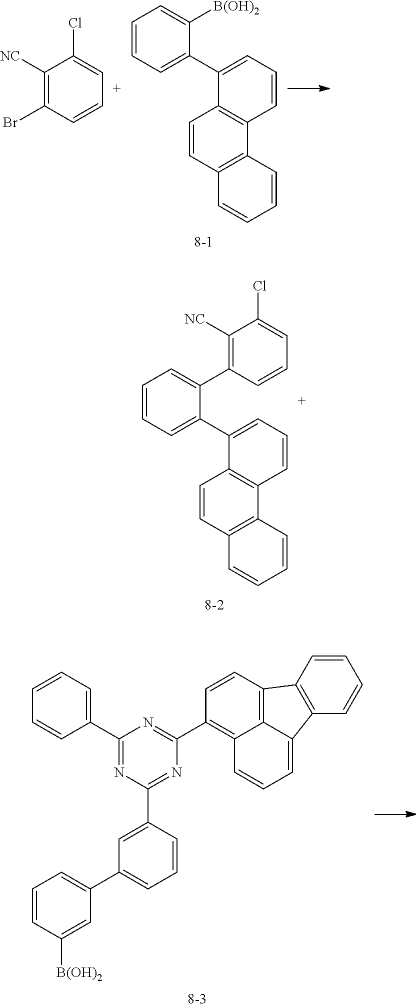

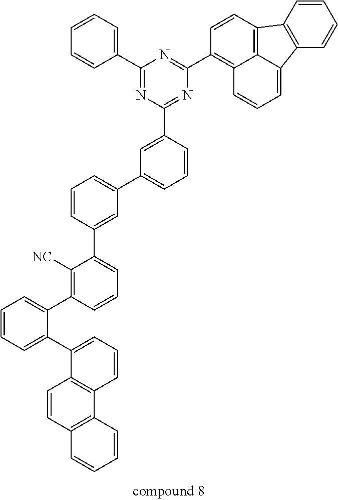 [SMILES: N#Cc1c(-c2cccc(-c3cccc(-c4nc(-c5ccccc5)nc(-c5ccc6c7c(cccc57)-c5ccccc5-6)n4)c3)c2)cccc1-c1ccccc1-c1cccc2c1ccc1ccccc12]

As shown in the above reaction equation, after preparing Compound 8-2 using Compound 8-1 instead of Compound 1-1, Compound 8 was prepared in the same manner as in Synthesis Example 1, except for using Compound 8-3 instead of Compound 1-3.

MS: [M+H]⁺=863.

### Synthesis Example 9. Synthesis of Compound 9

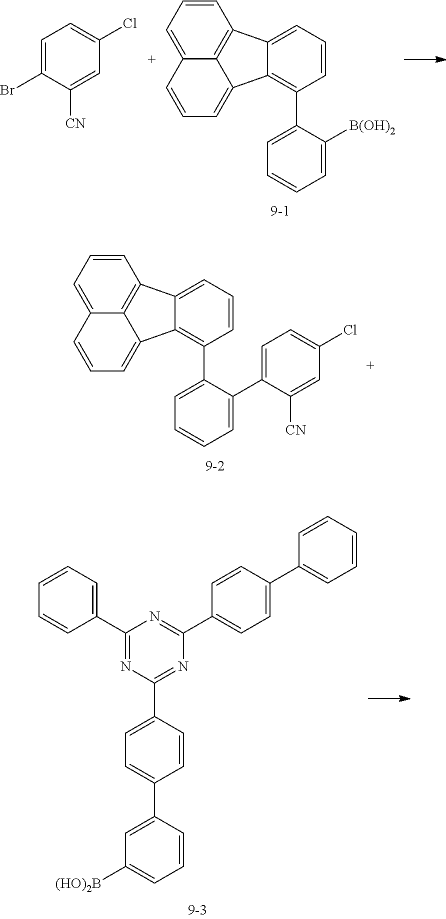

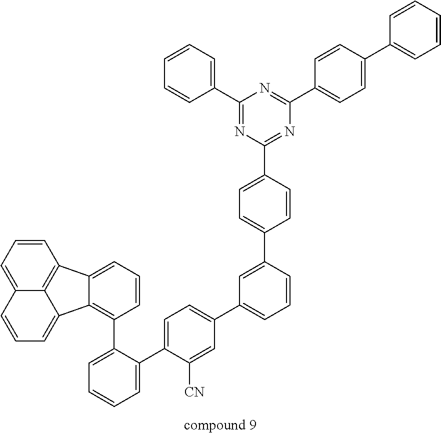 [SMILES: N#Cc1cc(-c2cccc(-c3ccc(-c4nc(-c5ccccc5)nc(-c5ccc(-c6ccccc6)cc5)n4)cc3)c2)ccc1-c1ccccc1-c1cccc2c1-c1cccc3cccc-2c13]

As shown in the above reaction scheme, after preparing Compound 9-2 using Compound 9-1 instead of Compound 1-1, Compound 9 was prepared in the same manner as in Synthesis Example 1, except for using Compound 9-3 instead of Compound 1-3.

MS: [M+H]⁺=839.

### Synthesis Example 10. Synthesis of Compound 10

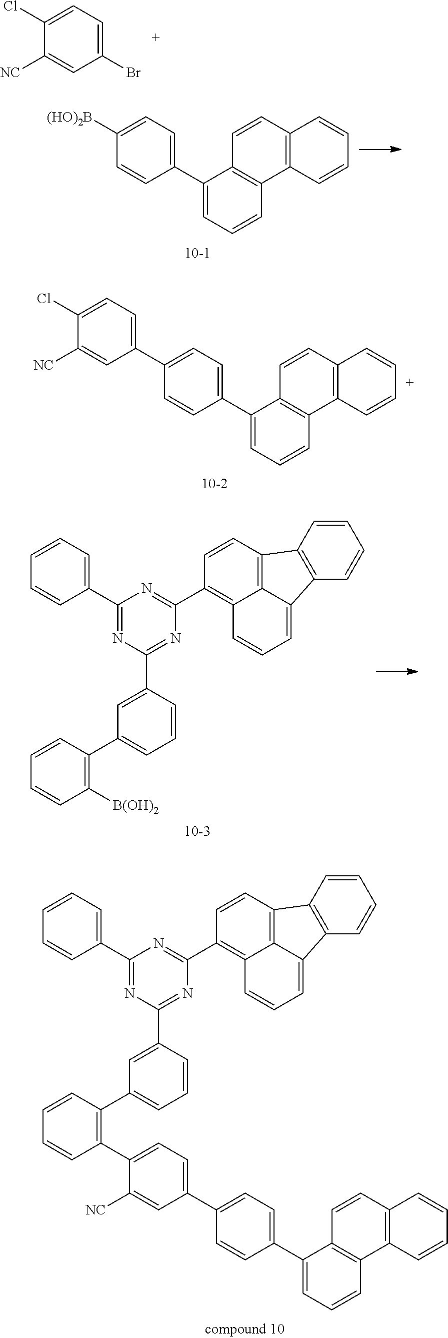

As shown in the above reaction scheme, after preparing Compound 10-2 using Compound 10⁻¹ instead of Compound 1-1, Compound 10 was prepared in the same manner as in Synthesis Example 1, except for using Compound 10⁻³ instead of Compound 1-3.

MS: [M+H]⁺=863.

### Synthesis Example 11. Synthesis of Compound 11

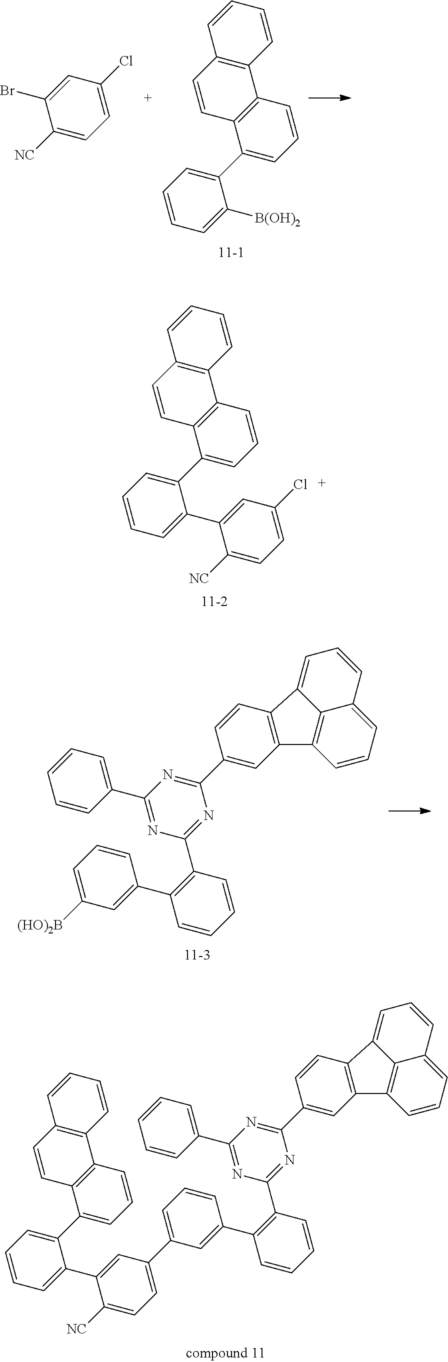 [SMILES: N#Cc1ccc(Cl)cc1Br ; OB(O)c1ccccc1-c1cccc2c1ccc1ccccc12 ; N#Cc1ccc(Cl)cc1-c1ccccc1-c1cccc2c1ccc1ccccc12 ; OB(O)c1cccc(-c2ccccc2-c2nc(-c3ccccc3)nc(-c3ccc4c(c3)-c3cccc5cccc-4c35)n2)c1 ; N#Cc1ccc(-c2cccc(-c3ccccc3-c3nc(-c4ccccc4)nc(-c4ccc5c(c4)-c4cccc6cccc-5c46)n3)c2)cc1-c1ccccc1-c1cccc2c1ccc1ccccc12]

As shown in the above reaction scheme, after preparing Compound 11-2 using Compound 11-1 instead of Compound 1-1, Compound 11 was prepared in the same manner as in Synthesis Example 1, except for using Compound 11-3 instead of Compound 1-3.

MS: [M+H]⁺=863.

### Synthesis Example 12. Synthesis of Compound 12

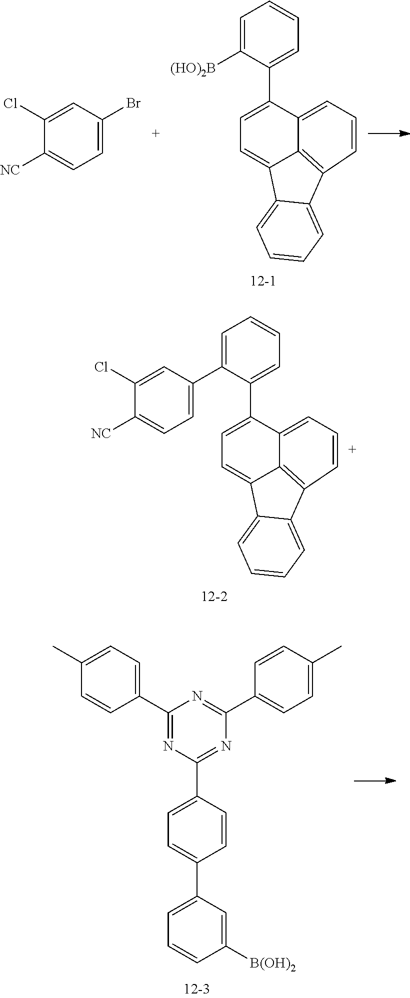

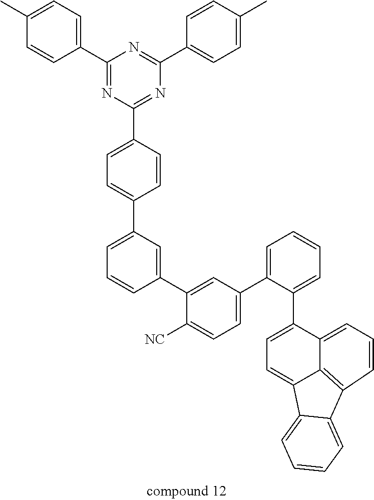 [SMILES: Cc1ccc(-c2nc(-c3ccc(C)cc3)nc(-c3ccc(-c4cccc(-c5cc(-c6ccccc6-c6ccc7c8c(cccc68)-c6ccccc6-7)ccc5C#N)c4)cc3)n2)cc1]

As shown in the above reaction scheme, after preparing Compound 12-2 using Compound 12-1 instead of Compound 1-1, Compound 12 was prepared in the same manner as in Synthesis Example 1, except for using Compound 12-3 instead of Compound 1-3.

MS: [M+H]⁺=791.

### Synthesis Example 13. Synthesis of Compound 13

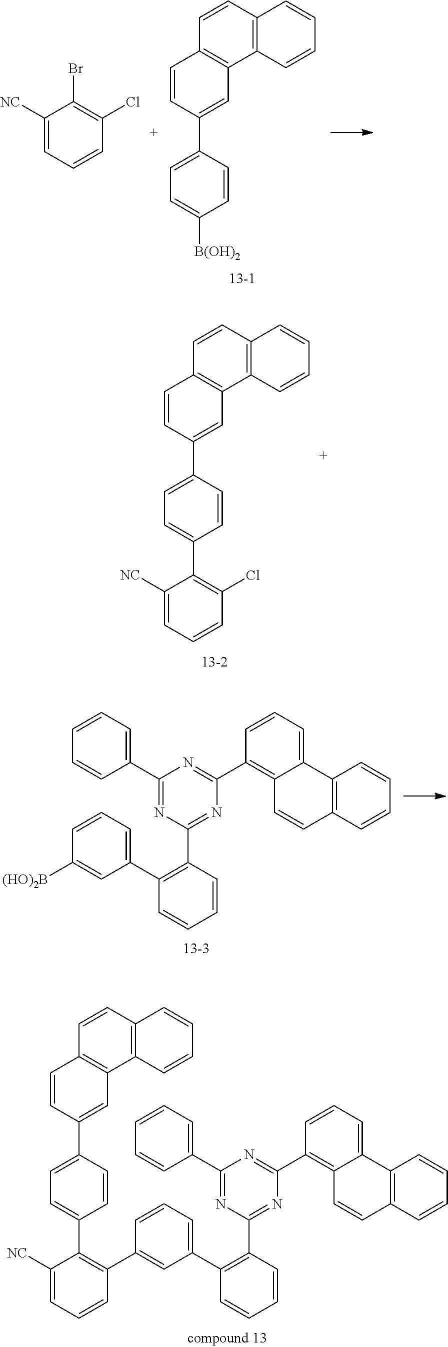

As shown in the above reaction scheme, after preparing Compound 13-2 using Compound 13-1 instead of Compound 1-1, Compound 13 was prepared in the same manner as in the preparation method of Synthesis Example 1, except for using Compound 13-3 instead of Compound 1-3.

MS: [M+H]⁺=839.

### Synthesis Example 14. Synthesis of Compound 14

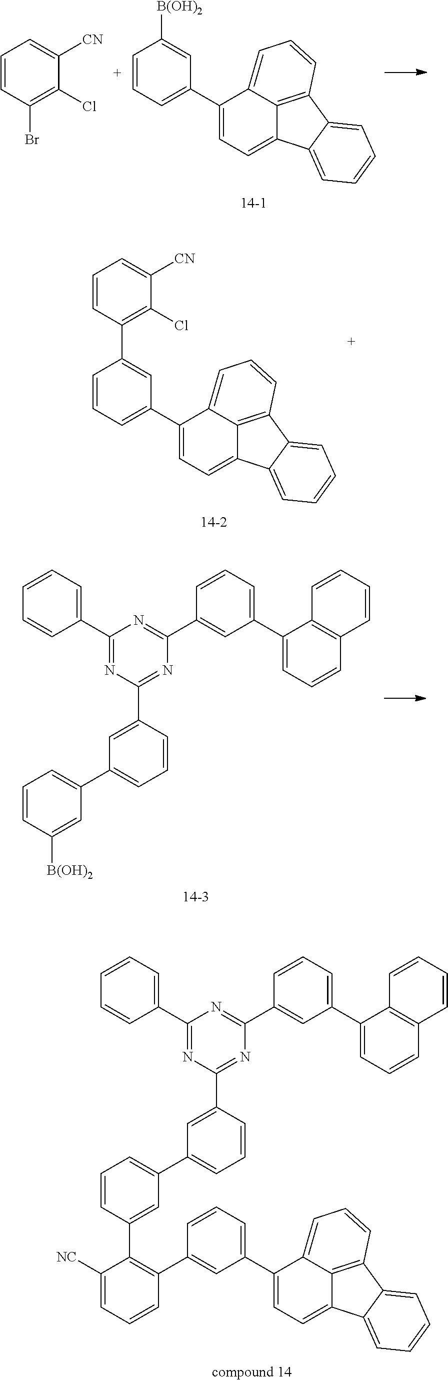 [SMILES: N#Cc1cccc(Br)c1Cl ; OB(O)c1cccc(-c2ccc3c4c(cccc24)-c2ccccc2-3)c1 ; N#Cc1cccc(-c2cccc(-c3ccc4c5c(cccc35)-c3ccccc3-4)c2)c1Cl ; OB(O)c1cccc(-c2cccc(-c3nc(-c4ccccc4)nc(-c4cccc(-c5cccc6ccccc56)c4)n3)c2)c1 ; N#Cc1cccc(-c2cccc(-c3ccc4c5c(cccc35)-c3ccccc3-4)c2)c1-c1cccc(-c2cccc(-c3nc(-c4ccccc4)nc(-c4cccc(-c5cccc6ccccc56)c4)n3)c2)c1]

As shown in the above reaction scheme, after preparing Compound 14-2 using Compound 14-1 instead of Compound 1-1, Compound 14 was prepared in the same manner as in the preparation method of Synthesis Example 1, except for using Compound 14-3 instead of Compound 1-3.

MS: [M+H]⁺=889.

### Synthesis Example 15. Synthesis of Compound 15

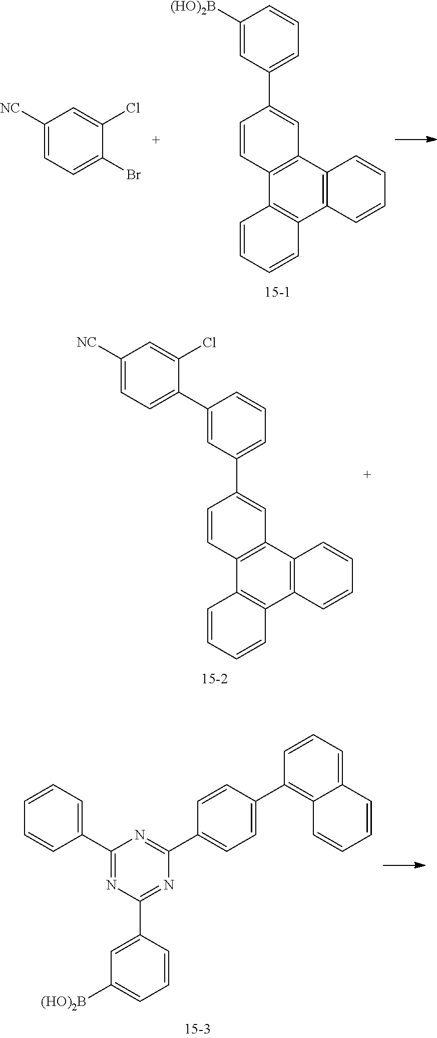 [SMILES: N#Cc1ccc(Br)c(Cl)c1 ; OB(O)c1cccc(-c2ccc3c4ccccc4c4ccccc4c3c2)c1 ; N#Cc1ccc(-c2cccc(-c3ccc4c5ccccc5c5ccccc5c4c3)c2)c(Cl)c1 ; OB(O)c1cccc(-c2nc(-c3ccccc3)nc(-c3ccc(-c4cccc5ccccc45)cc3)n2)c1]

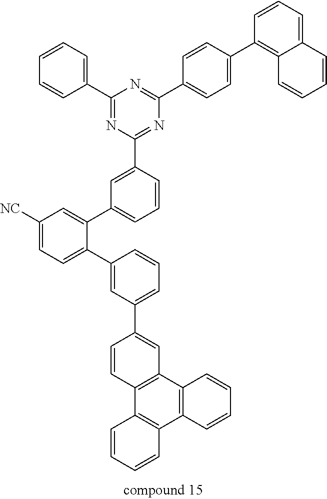 [SMILES: N#Cc1ccc(-c2cccc(-c3ccc4c5ccccc5c5ccccc5c4c3)c2)c(-c2cccc(-c3nc(-c4ccccc4)nc(-c4ccc(-c5cccc6ccccc56)cc4)n3)c2)c1]

As shown in the above reaction scheme, after preparing Compound 15-2 using Compound 15-1 instead of Compound 1-1, Compound 15 was prepared in the same manner as in Synthesis Example 1, except for using Compound 15-3 instead of Compound 1-3.

MS: [M+H]⁺=839.

### Synthesis Example 16. Synthesis of Compound 16

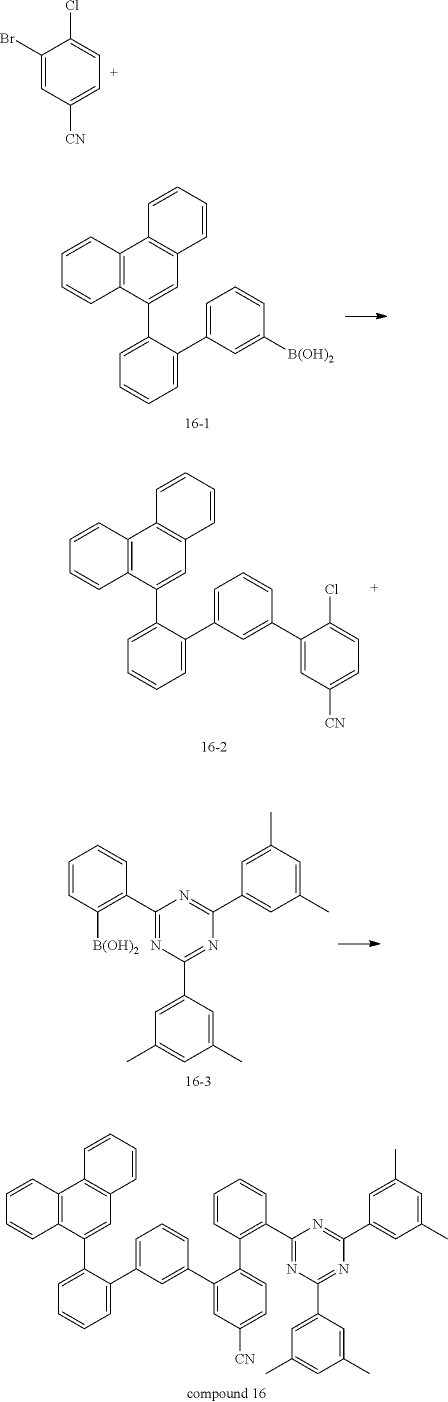 [SMILES: N#Cc1ccc(Cl)c(Br)c1 ; OB(O)c1cccc(-c2ccccc2-c2cc3ccccc3c3ccccc23)c1 ; N#Cc1ccc(Cl)c(-c2cccc(-c3ccccc3-c3cc4ccccc4c4ccccc34)c2)c1 ; Cc1cc(C)cc(-c2nc(-c3cc(C)cc(C)c3)nc(-c3ccccc3B(O)O)n2)c1 ; Cc1cc(C)cc(-c2nc(-c3cc(C)cc(C)c3)nc(-c3ccccc3-c3ccc(C#N)cc3-c3cccc(-c4ccccc4-c4cc5ccccc5c5ccccc45)c3)n2)c1]

As shown in the above reaction scheme, after preparing Compound 16-2 using Compound 16-1 instead of Compound 1-1, Compound 16 was prepared in the same manner as in Synthesis Example 1, except for using Compound 16-3 instead of Compound 1-3.

MS: [M+H]⁺=795.

### Synthesis Example 17. Synthesis of Compound 17

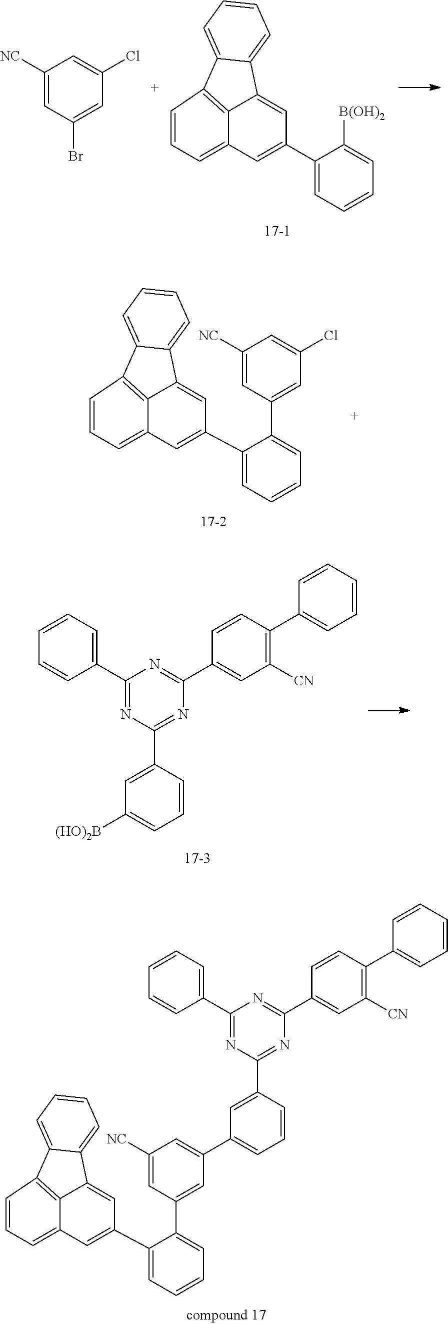 [SMILES: N#Cc1cc(Cl)cc(Br)c1 ; OB(O)c1ccccc1-c1cc2c3c(cccc3c1)-c1ccccc1-2 ; N#Cc1cc(Cl)cc(-c2ccccc2-c2cc3c4c(cccc4c2)-c2ccccc2-3)c1 ; N#Cc1cc(-c2nc(-c3ccccc3)nc(-c3cccc(B(O)O)c3)n2)ccc1-c1ccccc1 ; N#Cc1cc(-c2cccc(-c3nc(-c4ccccc4)nc(-c4ccc(-c5ccccc5)c(C#N)c4)n3)c2)cc(-c2ccccc2-c2cc3c4c(cccc4c2)-c2ccccc2-3)c1]

As shown in the above reaction scheme, after preparing Compound 17-2 using Compound 17-1 instead of Compound 1-1, Compound 17 was prepared in the same manner as in Synthesis Example 1, except for using Compound 17-3 instead of Compound 1-3.

MS: [M+H]⁺=788.

### Synthesis Example 18. Synthesis of Compound 18

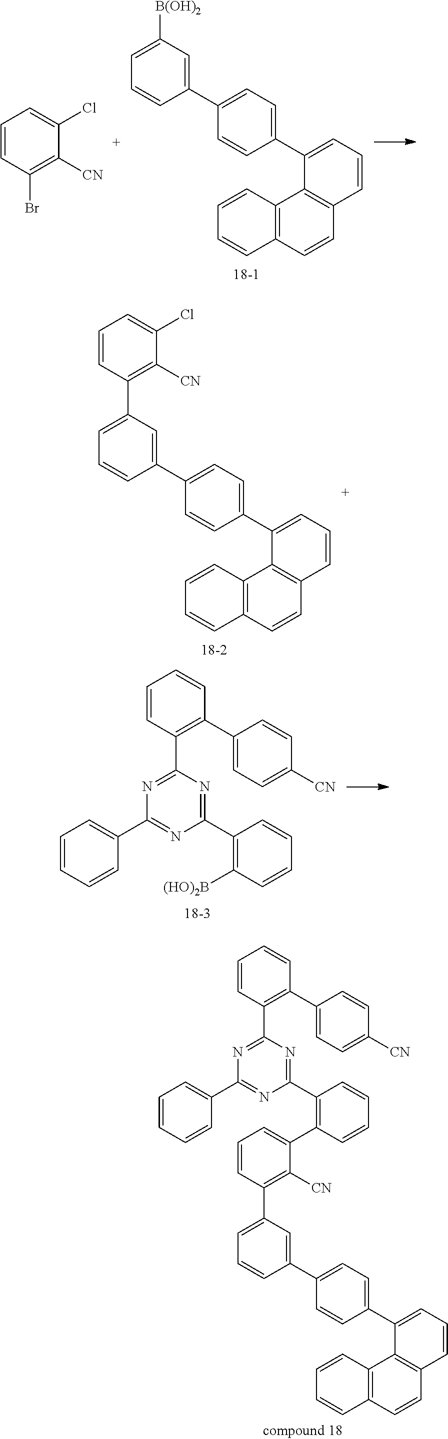 [SMILES: N#Cc1c(Cl)cccc1Br ; OB(O)c1cccc(-c2ccc(-c3cccc4ccc5ccccc5c34)cc2)c1 ; N#Cc1c(Cl)cccc1-c1cccc(-c2ccc(-c3cccc4ccc5ccccc5c34)cc2)c1 ; N#Cc1ccc(-c2ccccc2-c2nc(-c3ccccc3)nc(-c3ccccc3B(O)O)n2)cc1 ; N#Cc1ccc(-c2ccccc2-c2nc(-c3ccccc3)nc(-c3ccccc3-c3cccc(-c4cccc(-c5ccc(-c6cccc7ccc8ccccc8c67)cc5)c4)c3C#N)n2)cc1]

As shown in the above reaction scheme, after preparing Compound 18-2 using Compound 18-1 instead of Compound 1-1, Compound 18 was prepared in the same manner as in Synthesis Example 1, except for using Compound 18-3 instead of Compound 1-3.

MS: [M+H]⁺=840.

### Synthesis Example 19. Synthesis of Compound 19

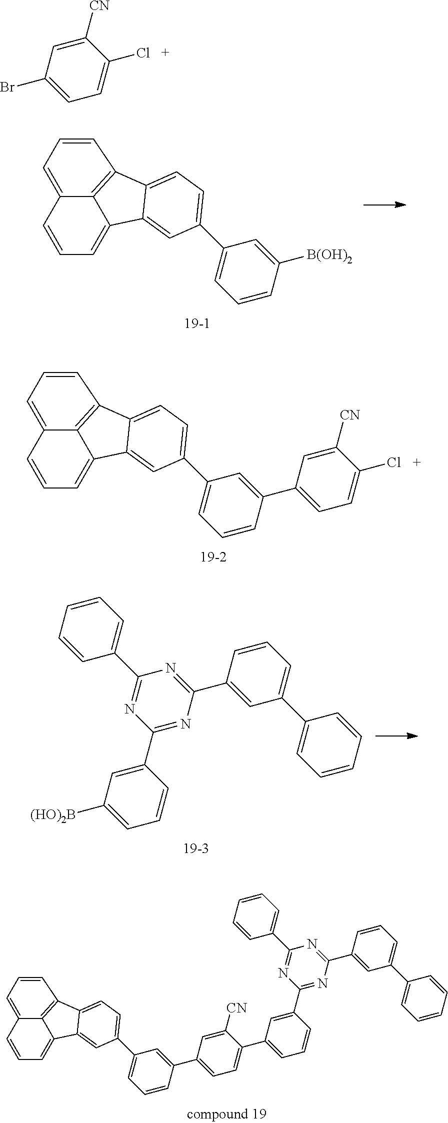

As shown in the above reaction scheme, after preparing Compound 19-2 using Compound 19-1 instead of Compound 1-1, Compound 19 was prepared in the same manner as in Synthesis Example 1, except for using Compound 19-3 instead of Compound 1-3.

MS: [M+H]⁺=763.

### Synthetic Example 20. Synthesis of Compound 20

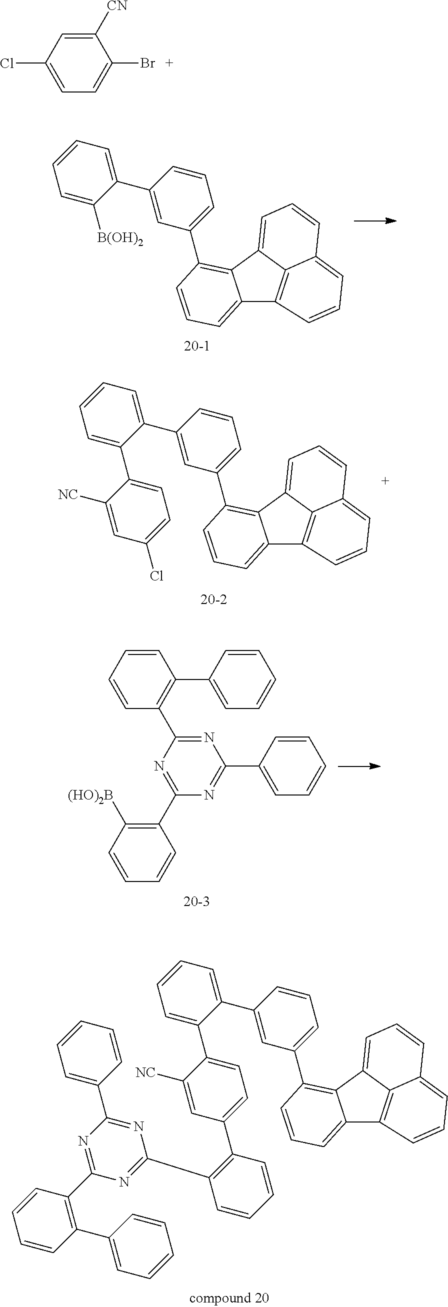 [SMILES: N#Cc1cc(Cl)ccc1Br ; OB(O)c1ccccc1-c1cccc(-c2cccc3c2-c2cccc4cccc-3c24)c1 ; N#Cc1cc(Cl)ccc1-c1ccccc1-c1cccc(-c2cccc3c2-c2cccc4cccc-3c24)c1 ; OB(O)c1ccccc1-c1nc(-c2ccccc2)nc(-c2ccccc2-c2ccccc2)n1 ; N#Cc1cc(-c2ccccc2-c2nc(-c3ccccc3)nc(-c3ccccc3-c3ccccc3)n2)ccc1-c1ccccc1-c1cccc(-c2cccc3c2-c2cccc4cccc-3c24)c1]

As shown in the above reaction scheme, after preparing Compound 20-2 using Compound 20-1 instead of Compound 1-1, Compound 20 was prepared in the same manner as in the preparation method of Synthesis Example 1, except for using Compound 20-3 instead of Compound 1-3.

MS: [M+H]⁺=839.

### EXAMPLES

### Example 1

A glass substrate on which ITO (Indium Tin Oxide) was coated as a thin film to a thickness of 1,000 Å (angstrom) was put into distilled water in which a detergent was dissolved, and ultrasonically cleaned. At this time, a product manufactured by Fischer Co. was used as the detergent, and distilled water filtered twice using a filter manufactured by Millipore Co. was used as the distilled water. After the ITO was cleaned for 30 minutes, ultrasonic cleaning was repeated twice using distilled water for 10 minutes. After the cleaning with distilled water was completed, the substrate was ultrasonically cleaned with solvents of isopropyl alcohol, acetone, and methanol, dried, and then transferred to a plasma cleaner. In addition, the substrate was cleaned for 5 minutes using oxygen plasma and then transferred to a vacuum depositor.

On the prepared ITO transparent electrode, the following Compound HI-A was thermally vacuum-deposited to a thickness of 600 Å to form a hole injection layer. Thereafter, the following compound HAT and the following compound HT-A were sequentially vacuum-deposited on the hole injection layer to a thickness of 50 Å and 60 Å, respectively, to form a first hole transport layer and a second hole transport layer. Then, the following Compound BH and the following Compound BD were vacuum-deposited on the second hole transport layer to a thickness of 200 Å in a weight ratio of 25:1 to form a light emitting layer. The Compound 1 prepared above and the following Compound LiQ were vacuum-deposited on the light emitting layer to a thickness of 350 Å in a weight ratio of 1:1 to form an electron transport and injection layer. Lithium fluoride (LiF) and aluminum were sequentially deposited on the electron transport and injection layer to a thickness of 10 Å and 1000 Å respectively to form a cathode, thereby manufacturing an organic light emitting device.

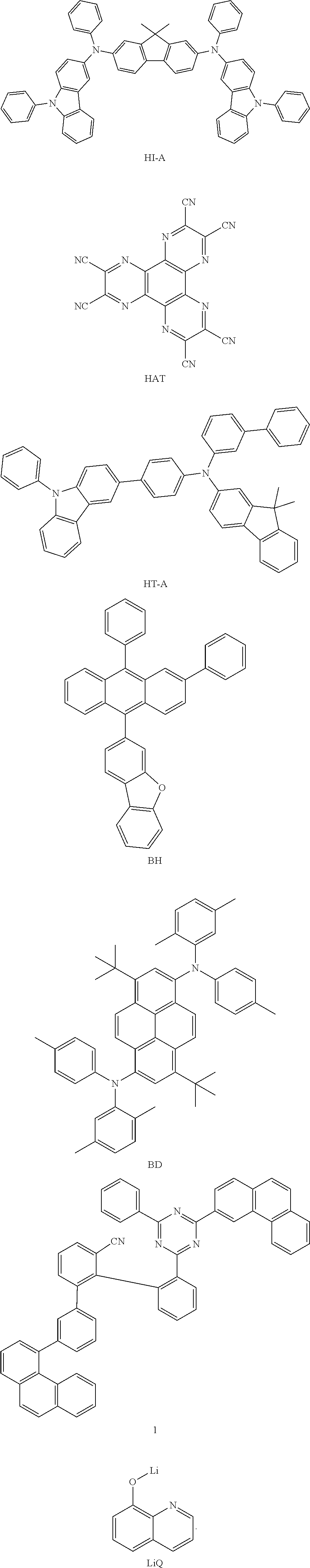

In the above process, the deposition rate of the organic material was maintained at 0.4 to 0.9 Å/sec, the deposition rate of lithium fluoride of cathode was maintained at 0.3 Å/sec, the deposition rate of aluminum was maintained at 2 Å/sec, and the degree of vacuum during the deposition was maintained at 1×10⁻⁷ to 5×10−5 torr, thereby manufacturing an organic light emitting device.

### Examples 2 to 20

An organic light emitting device was manufactured in the same manner as in Example 1, except that each of Compound 2 to 20 shown in Table 1 was used instead of Compound 1, respectively.

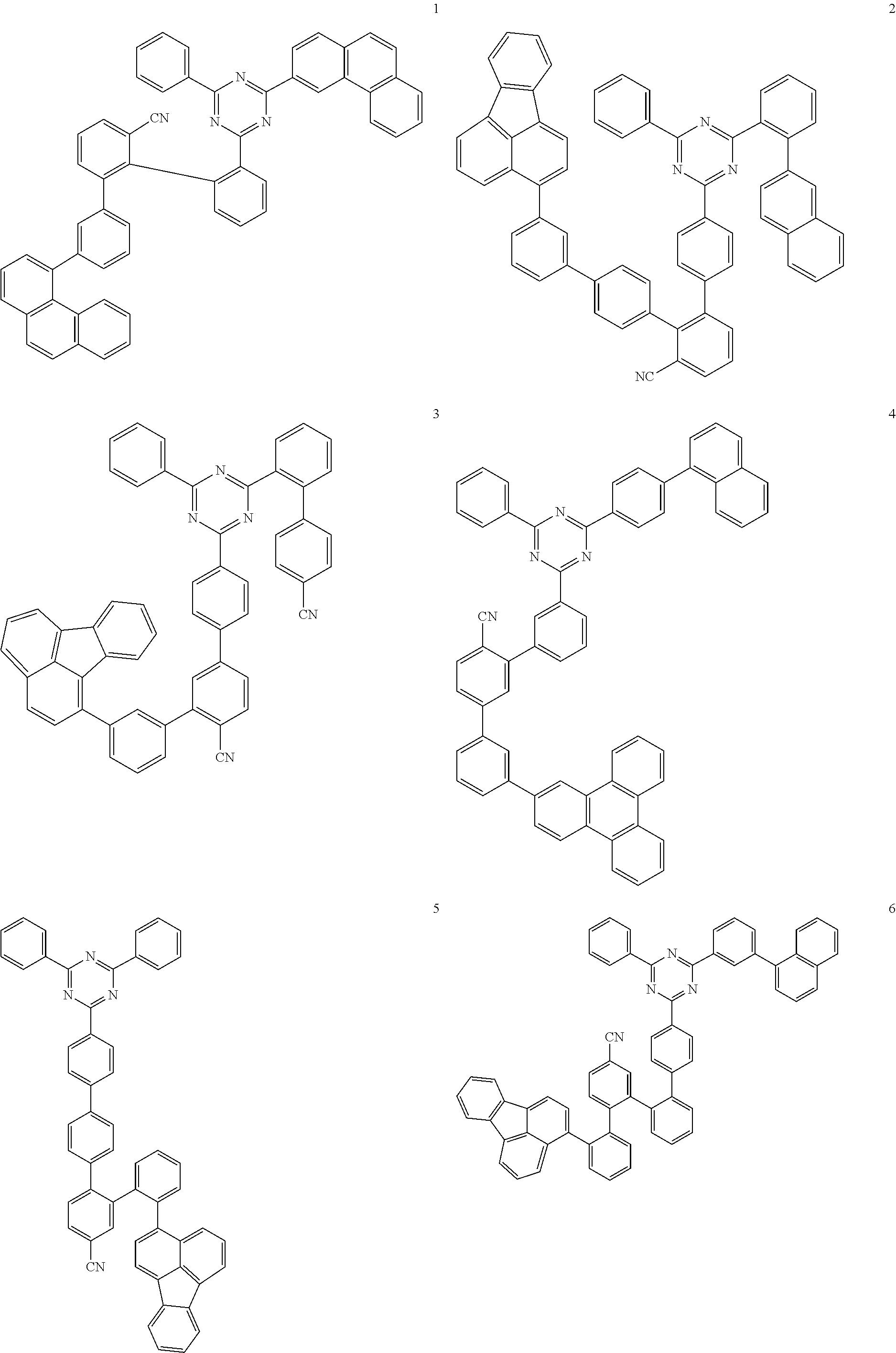

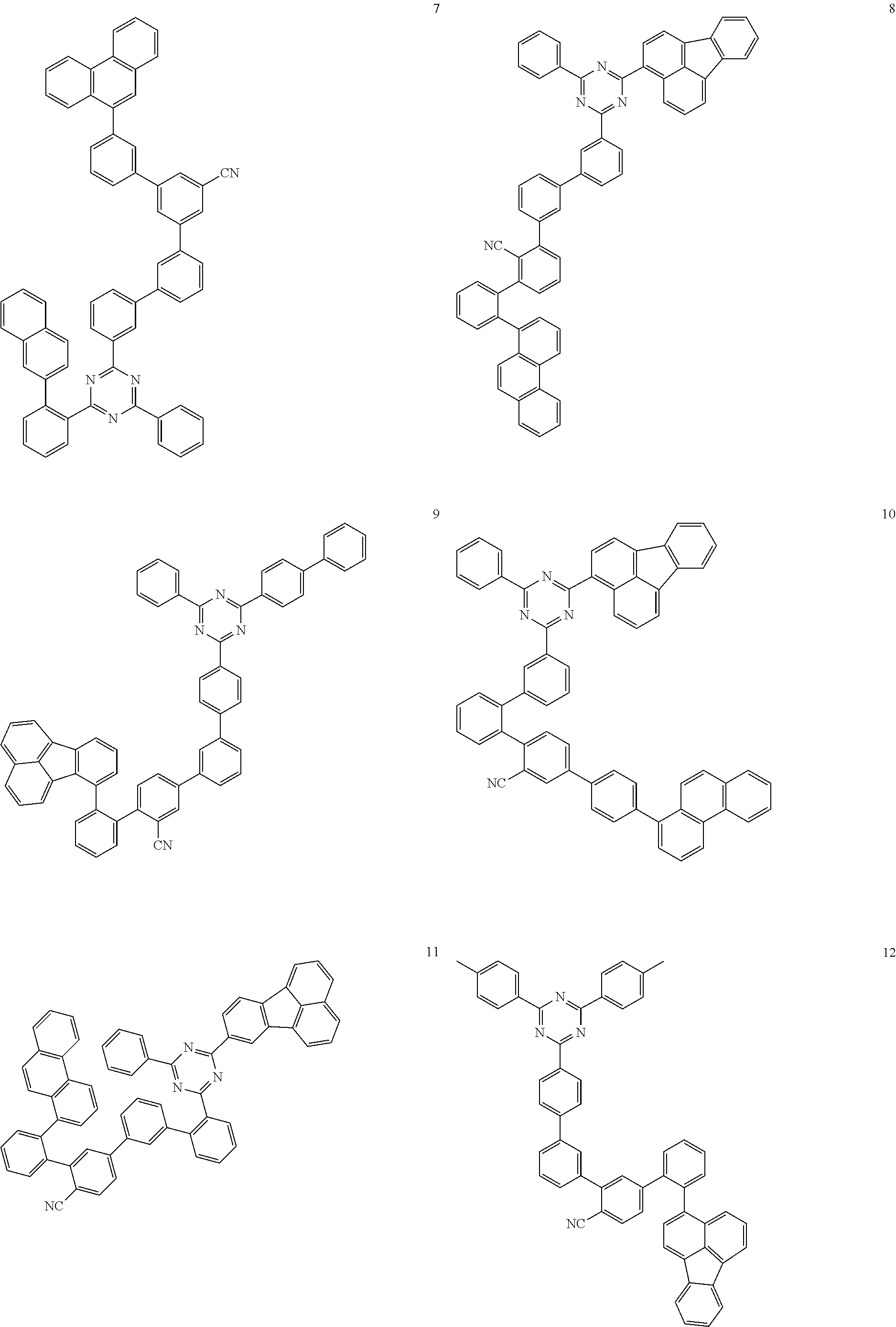

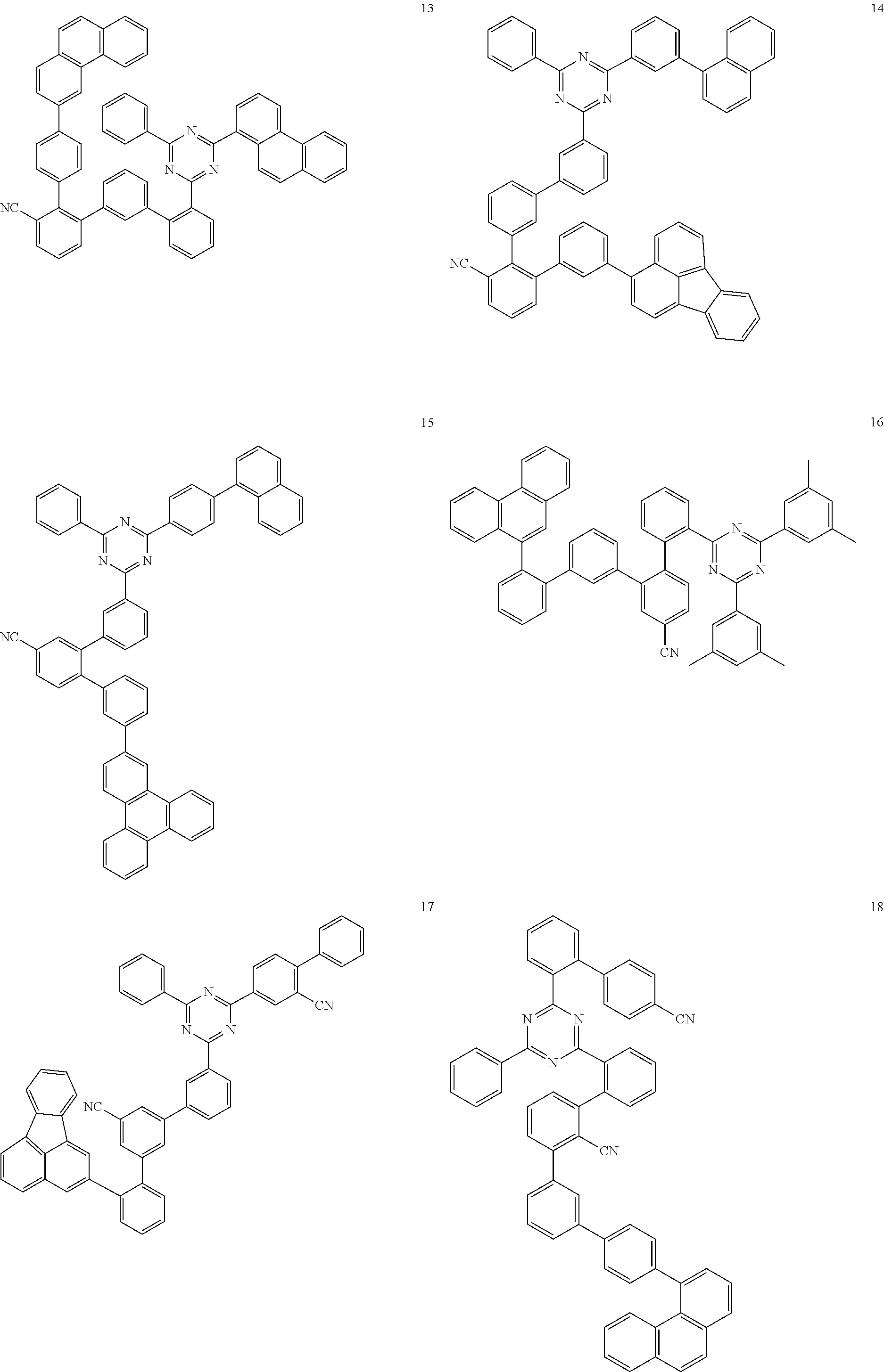

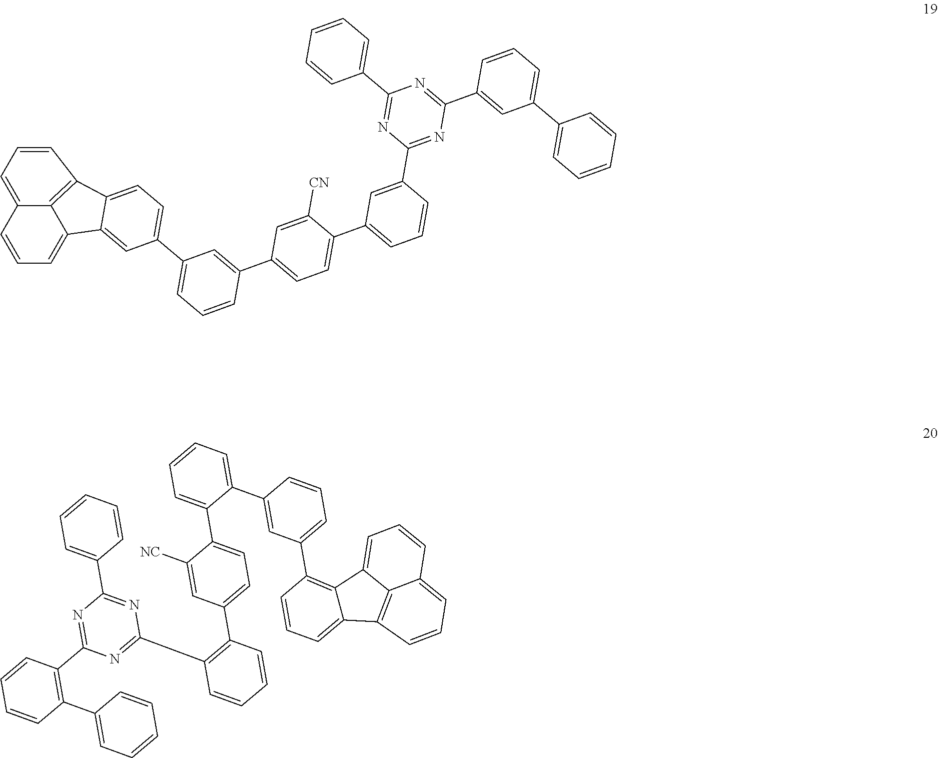 [SMILES: N#Cc1cc(-c2cccc(-c3ccc4c(c3)-c3cccc5cccc-4c35)c2)ccc1-c1cccc(-c2nc(-c3ccccc3)nc(-c3cccc(-c4ccccc4)c3)n2)c1 ; N#Cc1cc(-c2ccccc2-c2nc(-c3ccccc3)nc(-c3ccccc3-c3ccccc3)n2)ccc1-c1ccccc1-c1cccc(-c2cccc3c2-c2cccc4cccc-3c24)c1]

### Comparative Examples 1 to 10

An organic light emitting device was manufactured in the same manner as in Example 1, except that each of the following compounds shown in Table 1 was used instead of Compound 1, respectively. In Table 1, Compounds

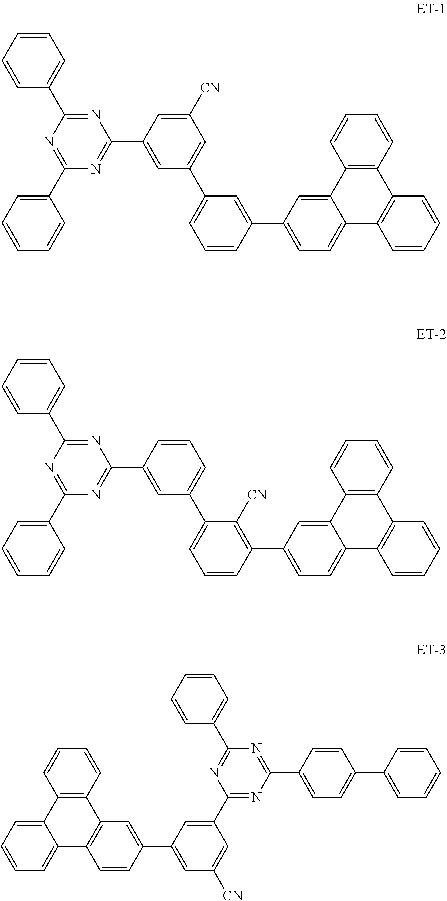 [SMILES: N#Cc1cc(-c2cccc(-c3ccc4c5ccccc5c5ccccc5c4c3)c2)cc(-c2nc(-c3ccccc3)nc(-c3ccccc3)n2)c1 ; N#Cc1c(-c2cccc(-c3nc(-c4ccccc4)nc(-c4ccccc4)n3)c2)cccc1-c1ccc2c3ccccc3c3ccccc3c2c1 ; N#Cc1cc(-c2ccc3c4ccccc4c4ccccc4c3c2)cc(-c2nc(-c3ccccc3)nc(-c3ccc(-c4ccccc4)cc3)n2)c1]

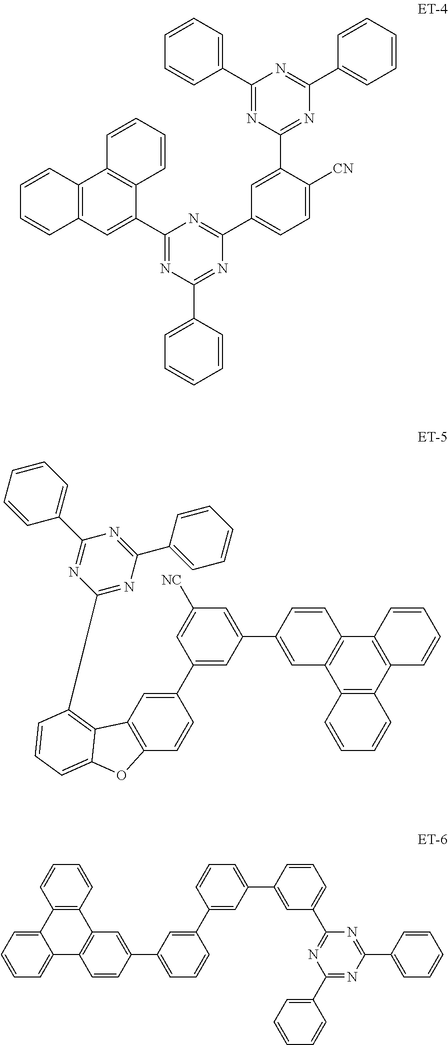 [SMILES: N#Cc1ccc(-c2nc(-c3ccccc3)nc(-c3cc4ccccc4c4ccccc34)n2)cc1-c1nc(-c2ccccc2)nc(-c2ccccc2)n1 ; N#Cc1cc(-c2ccc3c4ccccc4c4ccccc4c3c2)cc(-c2ccc3oc4cccc(-c5nc(-c6ccccc6)nc(-c6ccccc6)n5)c4c3c2)c1 ; c1ccc(-c2nc(-c3ccccc3)nc(-c3cccc(-c4cccc(-c5cccc(-c6ccc7c8ccccc8c8ccccc8c7c6)c5)c4)c3)n2)cc1]

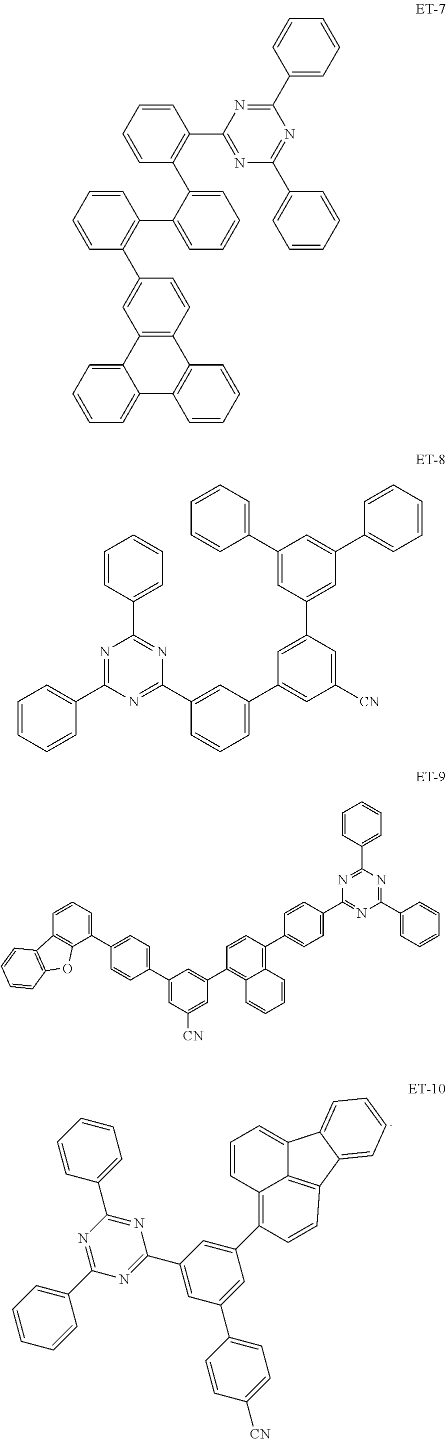 [SMILES: c1ccc(-c2nc(-c3ccccc3)nc(-c3ccccc3-c3ccccc3-c3ccccc3-c3ccc4c5ccccc5c5ccccc5c4c3)n2)cc1 ; N#Cc1cc(-c2cc(-c3ccccc3)cc(-c3ccccc3)c2)cc(-c2cccc(-c3nc(-c4ccccc4)nc(-c4ccccc4)n3)c2)c1 ; N#Cc1cc(-c2ccc(-c3cccc4c3oc3ccccc34)cc2)cc(-c2ccc(-c3ccc(-c4nc(-c5ccccc5)nc(-c5ccccc5)n4)cc3)c3ccccc23)c1 ; N#Cc1ccc(-c2cc(-c3nc(-c4ccccc4)nc(-c4ccccc4)n3)cc(-c3ccc4c5c(cccc35)-c3ccccc3-4)c2)cc1]

### Experimental Example

For the organic light emitting devices prepared in Examples and Comparative Examples, the voltage, efficiency, and lifespan (T95) were measured by applying a current, and the results are shown in Table 1 below. Herein, the voltage and efficiency were measured by applying a current density of 10 mA/cm². In addition, T95 in Table 1 below means the time taken the measured luminance decreases to 95% of the initial luminance at a current density of 20 mA/cm².

TABLE 1

| | | Voltage (V) | Efficiency (cd/A) | Coordinates | (hr) (T95 |
|---|---|---|---|---|---|
| | Compounds | (@10 mA/cm²) | (@10 mA/cm²) | (x, y) | at 20 mA/cm²) |
| Example 1 | Compound 1 | 4.09 | 5.00 | (0.140, 0.092) | 100 |
| Example 2 | Compound 2 | 4.06 | 5.08 | (0.140, 0.093) | 94 |
| Example 3 | Compound 3 | 4.13 | 4.92 | (0.140, 0.093) | 109 |
| Example 4 | Compound 4 | 4.09 | 5.04 | (0.141, 0.092) | 97 |
| Example 5 | Compound 5 | 4.21 | 4.75 | (0.140, 0.092) | 92 |
| Example 6 | Compound 6 | 4.15 | 4.82 | (0.140, 0.093) | 94 |
| Example 7 | Compound 7 | 4.02 | 5.12 | (0.140, 0.093) | 92 |
| Example 8 | Compound 8 | 4.13 | 4.96 | (0.141, 0.092) | 105 |
| Example 9 | Compound 9 | 4.24 | 4.79 | (0.140, 0.092) | 92 |
| Example 10 | Compound 10 | 4.29 | 4.76 | (0.140, 0.093) | 94 |
| Example 11 | Compound 11 | 4.21 | 4.83 | (0.140, 0.093) | 92 |
| Example 12 | Compound 12 | 4.28 | 4.71 | (0.140, 0.092) | 95 |
| Example 13 | Compound 13 | 4.29 | 4.72 | (0.140, 0.093) | 93 |
| Example 14 | Compound 14 | 4.36 | 4.64 | (0.140, 0.093) | 100 |
| Example 15 | Compound 15 | 4.21 | 4.79 | (0.140, 0.092) | 89 |
| Example 16 | Compound 16 | 4.33 | 4.68 | (0.140, 0.093) | 97 |
| Example 17 | Compound 17 | 4.29 | 4.72 | (0.140, 0.093) | 93 |
| Example 18 | Compound 18 | 4.21 | 4.79 | (0.141, 0.092) | 91 |
| Example 19 | Compound 19 | 4.24 | 4.75 | (0.140, 0.092) | 92 |
| Example 20 | Compound 20 | 4.24 | 4.75 | (0.140, 0.093) | 95 |
| Comparative | ET-1 | 4.18 | 3.71 | (0.140, 0.093) | 72 |
| Example 1 | | | | | |
| Comparative | ET-2 | 4.15 | 3.95 | (0.141, 0.092) | 80 |
| Example 2 | | | | | |
| Comparative | ET-3 | 4.20 | 3.10 | (0.140, 0.092) | 73 |
| Example 3 | | | | | |
| Comparative | ET-4 | 4.21 | 4.19 | (0.140, 0.093) | 10 |
| Example 4 | | | | | |
| Comparative | ET-5 | 4.20 | 4.13 | (0.140, 0.093) | 15 |
| Example 5 | | | | | |
| Comparative | ET-6 | 4.48 | 2.44 | (0.141, 0.092) | 49 |
| Example 6 | | | | | |
| Comparative | ET-7 | 4.54 | 2.40 | (0.140, 0.093) | 48 |
| Example 7 | | | | | |
| Comparative | ET-8 | 4.40 | 2.53 | (0.140, 0.093) | 57 |
| Example 8 | | | | | |
| Comparative | ET-9 | 4.43 | 4.14 | (0.140, 0.093) | 47 |
| Example 9 | | | | | |
| Comparative | ET-10 | 4.33 | 2.38 | (0.141, 0.094) | 80 |
| Example 10 | | | | | |

The results of Table 1 were obtained by applying a current to the organic light-emitting devices of Examples 1 to 20 and Comparative Examples 1 to 10. Specifically, as described above, the organic light-emitting device of Example 1 was manufactured by using Compound 1 and Compound LiQ for the electron injection and transport layer, with materials widely used conventionally as known in the art. In addition, the organic light-emitting devices of Comparative Examples 1 to 10 were manufactured by using each of Compounds ET-1 to ET-10 instead of Compound 1.

Referring to Table 1, it can be seen that, when the compounds of Chemical Formula 1 according to the present disclosure were used, the characteristics of low voltage, high efficiency, and long lifespan can be significantly improved. Namely, the organic light-emitting devices of Examples 1 to 20, manufactured by using the compounds having a specific polycyclic structure in which both a heterocyclic ring containing at least one N and an aromatic fused polycyclic ring having 14 to 60 carbon atoms are bonded to phenylene substituted with a cyano group via an arylene linker, respectively, in the electron injection and transport layer, show the higher efficiency and longer lifespan with maintaining lower voltage than those of the organic light-emitting devices of Comparative Examples 1 to 10 manufactured by using Compounds ET-1, ET-2, ET-3, ET-4, ET-5, ET-6, ET-7, ET-8, ET-9, and ET-10. Specifically, the organic light-emitting devices of Examples 1 to 20 show excellent characteristics with an improvement of about 10.7% to about 115.1% in efficiency and about 11.3% to about 990% in lifespan, while maintaining low voltage similar to or lower than those of the organic light-emitting devices of Comparative Examples 1 to 10 manufactured by using the compounds having a structure different from Chemical Formula 1, that is, in which a phenylene substituted with a cyano group is not included or the specific substituent of the phenylene substituted with a cyano group is not included. Thus, it can be considered that, the compounds of Examples 1 to 20 according to the present disclosure can efficiently transfer electrons from the cathode to the light-emitting layer with a high mobility for electrons, compared to the compounds of Comparative Examples 1 to 10.

In conclusion, it can be confirmed that the compounds according to the present disclosure can be effectively applied to the organic material layer responsible for electron injection or transport of organic light emitting devices, so that the characteristics of low voltage, high efficiency, and long lifespan can be significantly improved.

### DESCRIPTION OF SYMBOLS

| | |
|---|---|
| 1: Substrate | 2: Anode |
| 3: Light emitting layer | 4: Cathode |
| 5: Hole injection layer | 6: First hole transport layer |
| 7: Second hole transport layer | 8: Electron injection and transport layer |

1. A compound of Chemical Formula 1: 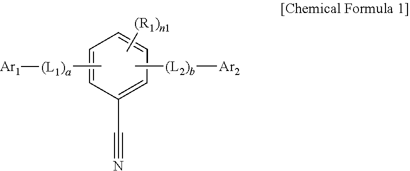
wherein in Chemical Formula 1;
L₁ and L₂ are each independently a substituted or unsubstituted C₆₋₆₀ arylene,
each R₁ is independently hydrogen, deuterium, halogen, cyano, substituted or unsubstituted C₁₋₆₀ alkyl, substituted or unsubstituted C₁₋₆₀ alkoxy, substituted or unsubstituted C₂₋₆₀ alkenyl, substituted or unsubstituted C₂₋₆₀ alkynyl, substituted or unsubstituted C₃₋₆₀ cycloalkyl, substituted or unsubstituted C₆₋₆₀ aryl, or substituted or unsubstituted C₂₋₆₀ heteroaryl containing any one or more heteroatoms selected from the group consisting of N, O and S;
a and b are each independently 1 or 2;
n1 is 3;
one of Ar₁ and Ar₂ is a substituent represented by the following Chemical Formula 2, and the other of Ar₁ and Ar₂ is a substituted or unsubstituted C₁₄₋₆₀ aromatic fused polycyclic ring; 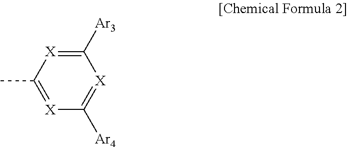
wherein in Chemical Formula 2;
each X is independently N or CH, provided that at least one X is N; and
Ar₃ and Ar₄ are each independently a substituted or unsubstituted C₆₋₆₀ aryl or substituted or unsubstituted C₂₋₆₀ heteroaryl containing any one or more heteroatoms selected from the group consisting of N, O and S.

2. The compound of claim 1, wherein the compound of Chemical Formula 1 is any one of the following Chemical Formula 1-1 to Chemical Formula 1-3: 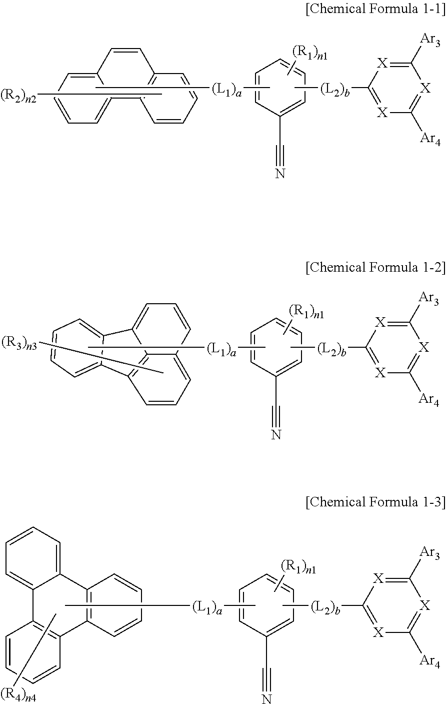
wherein in Chemical Formulae 1-1 to 1-3:
L₁, L₂, a, b, R₁, n1, X, Ar₃, and Ar₄ are as defined in claim 1;
R₂, R₃, and R₄ are each independently hydrogen, deuterium, halogen, cyano, substituted or unsubstituted C₁₋₆₀ alkyl, substituted or unsubstituted C₁₋₆₀ alkoxy, substituted or unsubstituted C₂₋₆₀ alkenyl, substituted or unsubstituted C₂₋₆₀ alkynyl, substituted or unsubstituted C₃₋₆₀ cycloalkyl, substituted or unsubstituted C₆₋₆₀ aryl, or substituted or unsubstituted C₂₋₆₀ heteroaryl containing any one or more heteroatoms selected from the group consisting of N, O and S;
n2 is 9;
n3 is 9; and
n4 is 11.

3. The compound of claim 1, wherein L₁ and L₂ are each independently substituted or unsubstituted C₆₋₂₀ arylene.

4. The compound of claim 1, wherein L₁ and L₂ are each independently phenylene, biphenylylene, or naphthylene.

5. The compound of claim 1, wherein at least one of L₁ and L₂ is phenylene, and the rest of L₁ and L₂ is phenylene or biphenylylene.

6. The compound of claim 1, wherein each R₁ is independently hydrogen, deuterium, C₁₋₆ alkyl, or C₆₋₁₂ aryl.

7. The compound of claim 1, wherein each R₁ is independently hydrogen or deuterium.

8. The compound of claim 1, wherein one of Ar₁ and Ar₂ is a substituent represented by Chemical Formula 2, and the other of Ar₁ and Ar₂ is substituted or unsubstituted phenanthrenyl, or substituted or unsubstituted triphenylenyl, or substituted or unsubstituted fluoranthenyl.

9. The compound of claim 1, wherein one of Ar₁ and Ar₂ is a substituent represented by Chemical Formula 2, and the other of Ar₁ and Ar₂ is any one substituent selected from the group consisting of the following substituents:

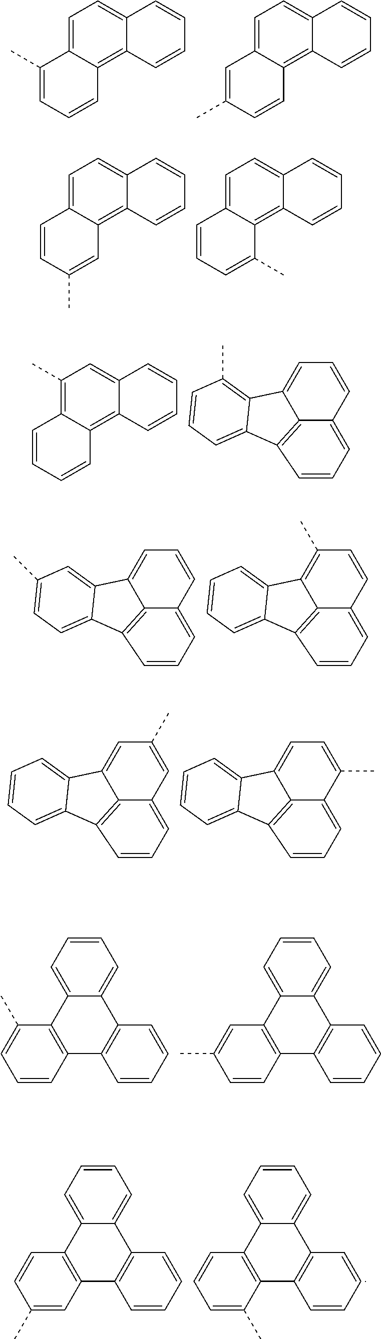

10. The compound of claim 1, wherein Ar₃ and Ar₄ are each independently a substituted or unsubstituted C₆₋₂₅ aryl or a substituted or unsubstituted C₁₂₋₁₈ heteroaryl.

11. The compound of claim 1, wherein Ar₃ and Ar₄ are each independently phenyl, phenyl substituted with one to five deuteriums, phenyl substituted with one to five methyls, phenyl substituted with one to five cyanyls, phenyl substituted with naphthyl, phenyl substituted with phenanthrenyl, phenyl substituted with triphenylenyl, phenyl substituted with fluoranthenyl, biphenyl, biphenyl substituted with one to nine deuteriums, biphenyl substituted with one to nine methyls, biphenyl substituted with one to nine cyanyls, terphenyl, quaterphenyl, naphthyl, naphthyl substituted with phenyl, phenanthrenyl, triphenylenyl, or fluoranthenyl.

12. The compound of claim 1, wherein Ar₃ and Ar₄ are each independently any one substituent selected from the group consisting of the following substituents:

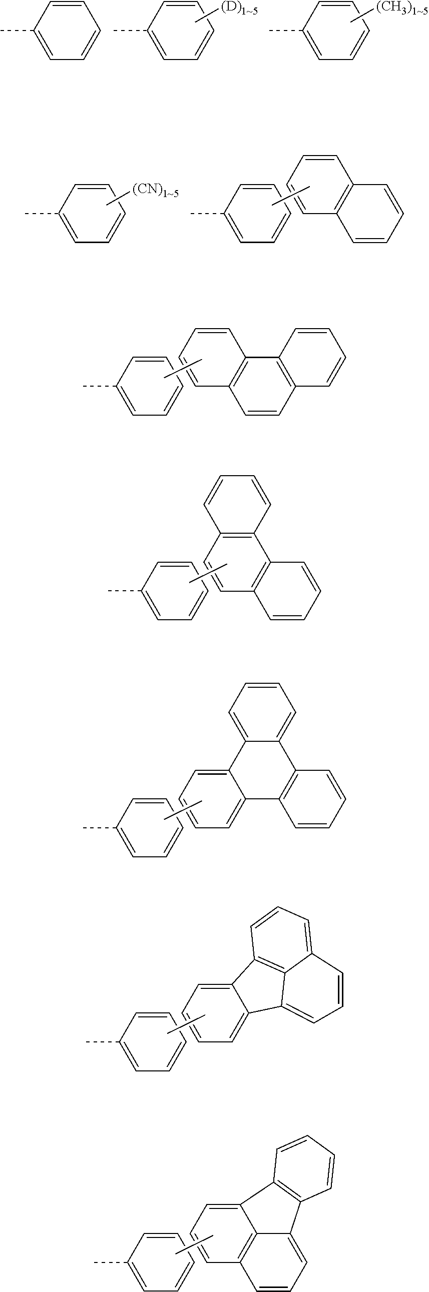

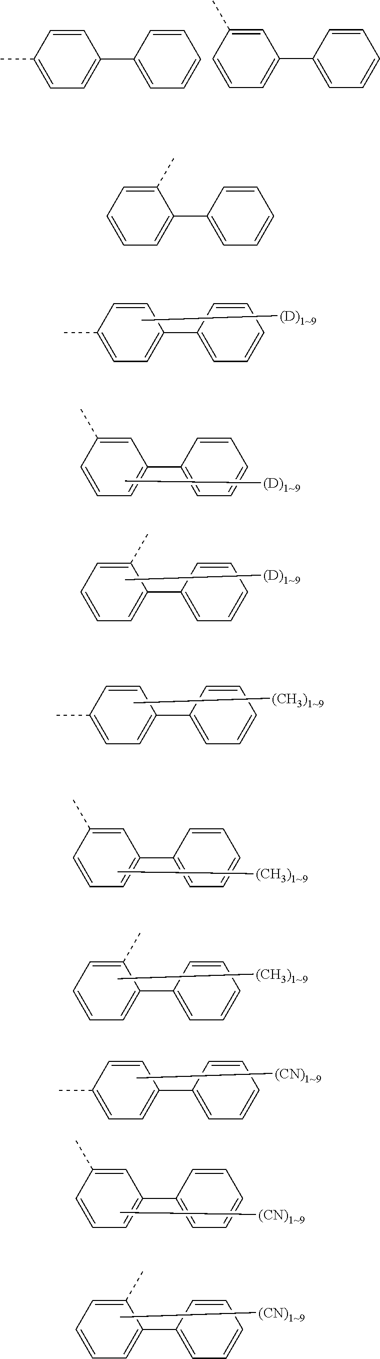

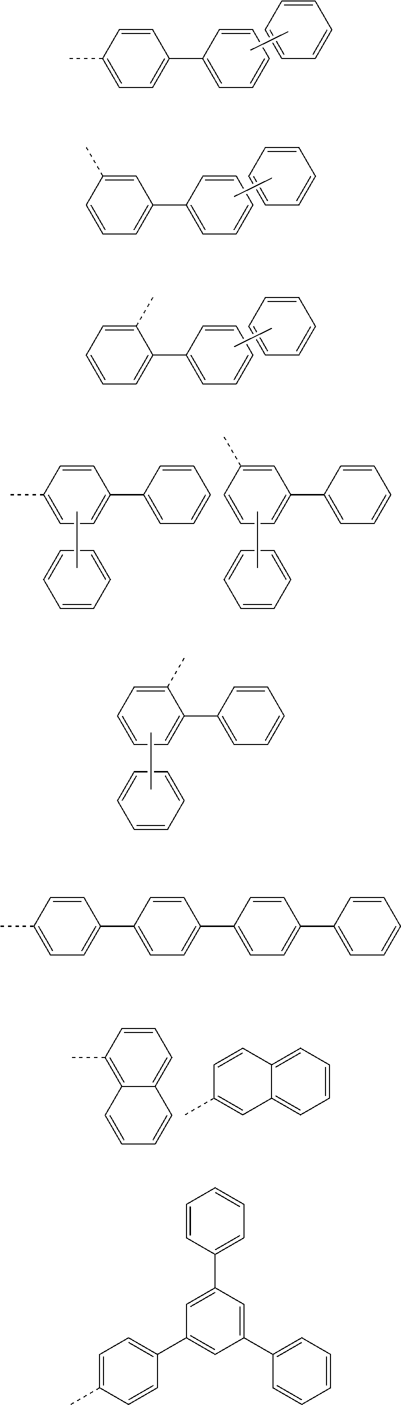

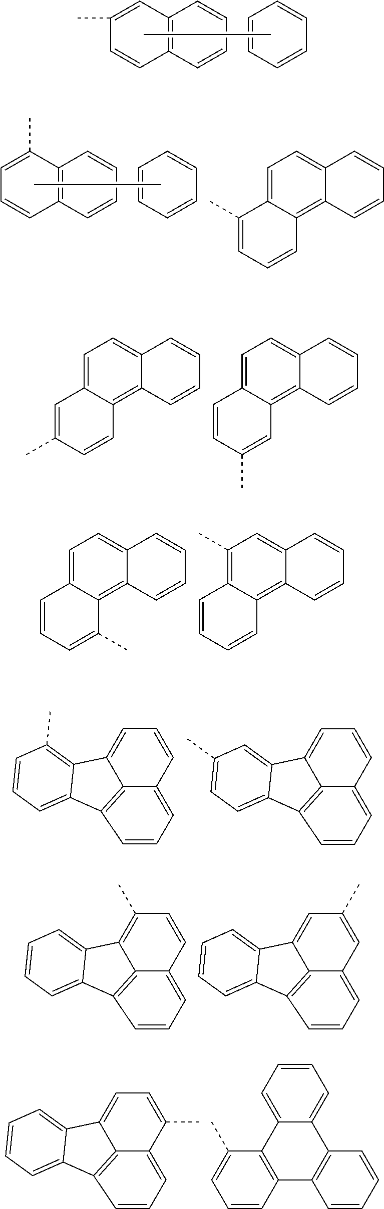

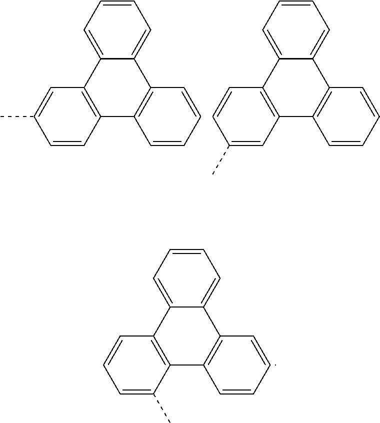

13. The compound of claim 1, wherein the compound of Chemical Formula 1 is any one compound selected from the group consisting of the following compounds:

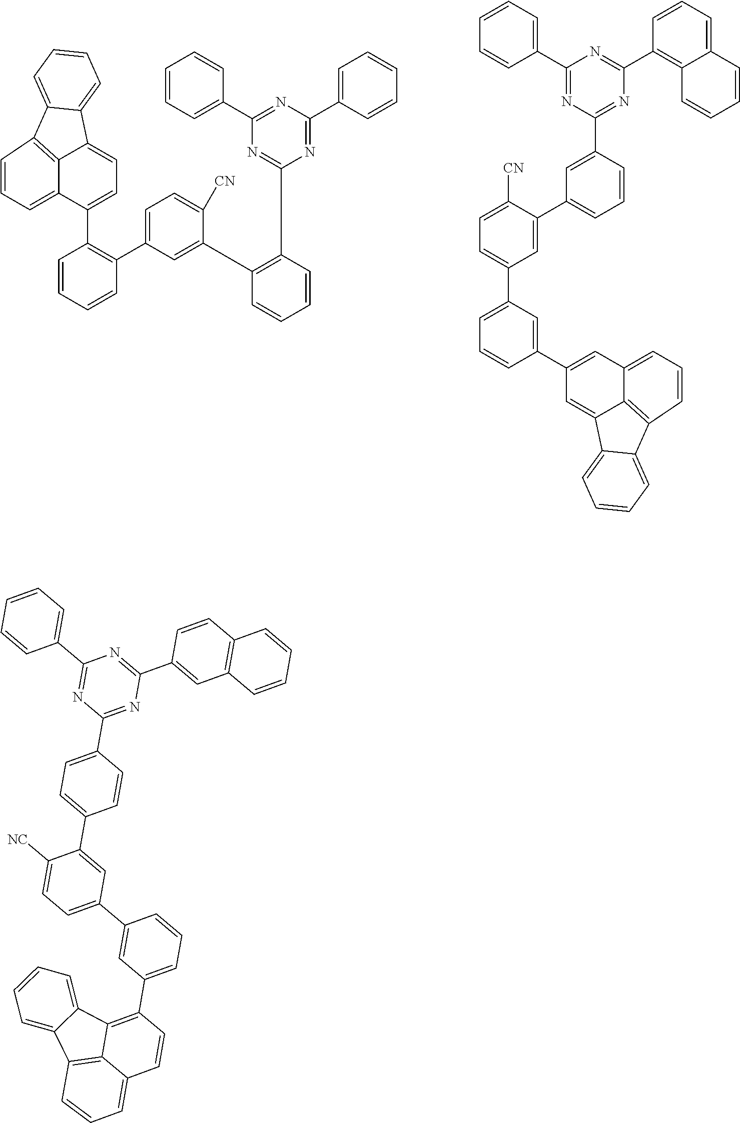 [SMILES: N#Cc1ccc(-c2ccccc2-c2ccc3c4c(cccc24)-c2ccccc2-3)cc1-c1ccccc1-c1nc(-c2ccccc2)nc(-c2ccccc2)n1 ; N#Cc1ccc(-c2cccc(-c3cc4c5c(cccc5c3)-c3ccccc3-4)c2)cc1-c1cccc(-c2nc(-c3ccccc3)nc(-c3cccc4ccccc34)n2)c1 ; N#Cc1ccc(-c2cccc(-c3ccc4cccc5c4c3-c3ccccc3-5)c2)cc1-c1ccc(-c2nc(-c3ccccc3)nc(-c3ccc4ccccc4c3)n2)cc1]

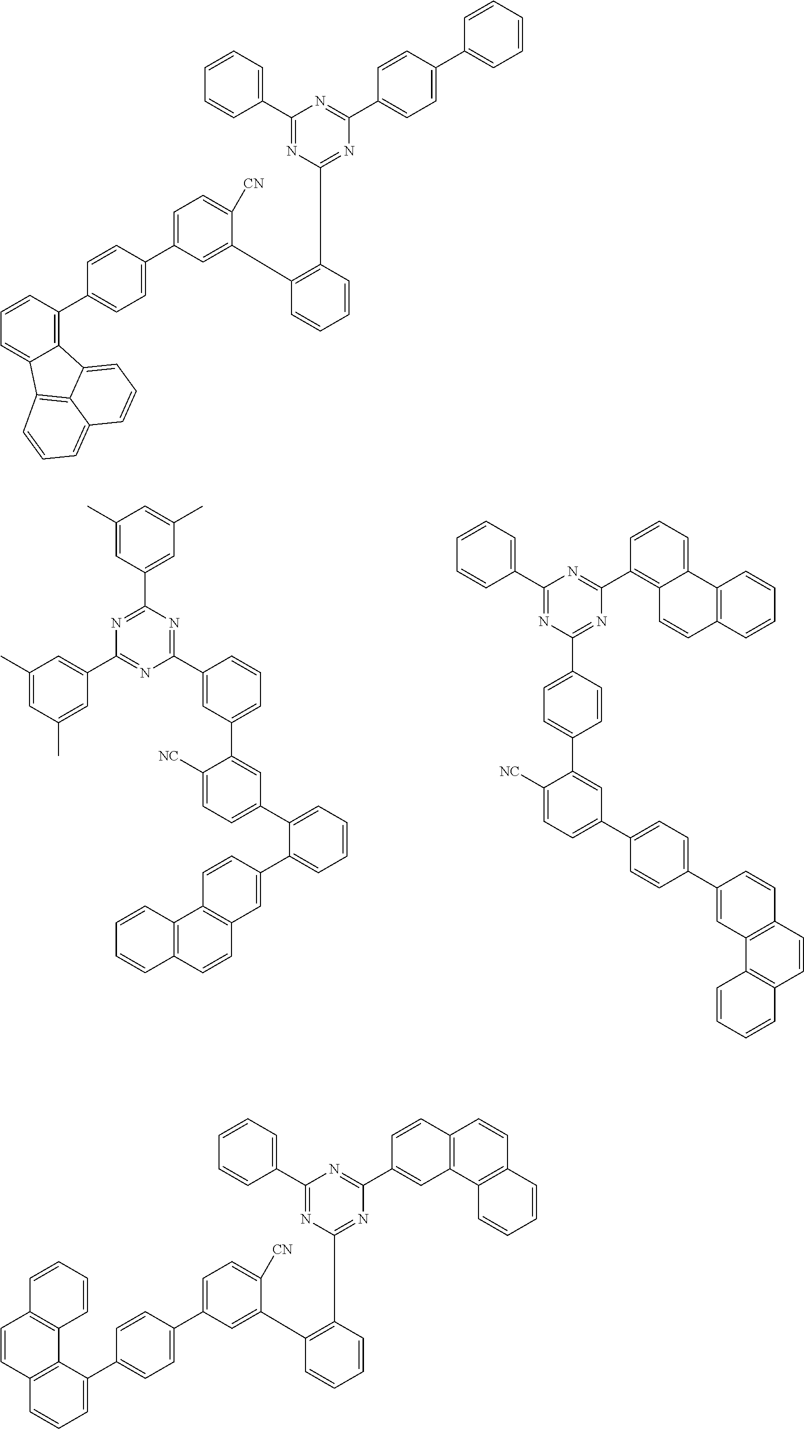 [SMILES: N#Cc1ccc(-c2ccc(-c3cccc4c3-c3cccc5cccc-4c35)cc2)cc1-c1ccccc1-c1nc(-c2ccccc2)nc(-c2ccc(-c3ccccc3)cc2)n1 ; Cc1cc(C)cc(-c2nc(-c3cc(C)cc(C)c3)nc(-c3cccc(-c4cc(-c5ccccc5-c5ccc6c(ccc7ccccc76)c5)ccc4C#N)c3)n2)c1 ; N#Cc1ccc(-c2ccc(-c3ccc4ccc5ccccc5c4c3)cc2)cc1-c1ccc(-c2nc(-c3ccccc3)nc(-c3cccc4c3ccc3ccccc34)n2)cc1 ; N#Cc1ccc(-c2ccc(-c3cccc4ccc5ccccc5c34)cc2)cc1-c1ccccc1-c1nc(-c2ccccc2)nc(-c2ccc3ccc4ccccc4c3c2)n1]

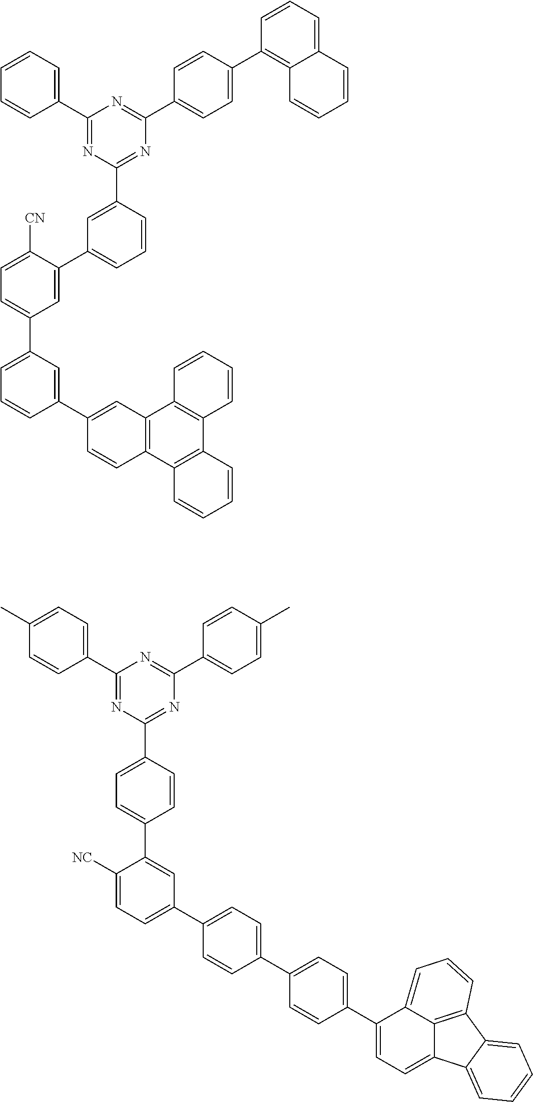 [SMILES: N#Cc1ccc(-c2cccc(-c3ccc4c5ccccc5c5ccccc5c4c3)c2)cc1-c1cccc(-c2nc(-c3ccccc3)nc(-c3ccc(-c4cccc5ccccc45)cc3)n2)c1 ; Cc1ccc(-c2nc(-c3ccc(C)cc3)nc(-c3ccc(-c4cc(-c5ccc(-c6ccc(-c7ccc8c9c(cccc79)-c7ccccc7-8)cc6)cc5)ccc4C#N)cc3)n2)cc1]

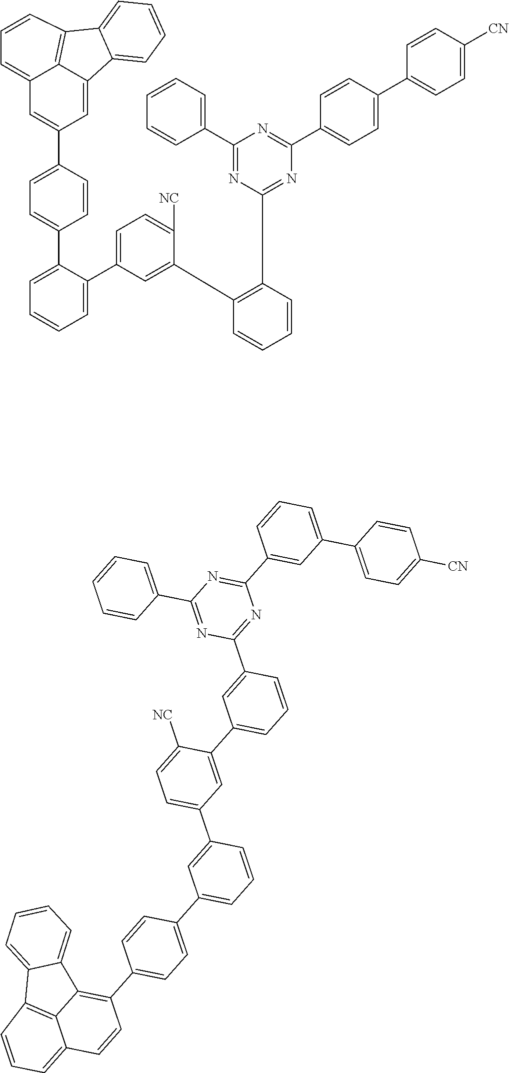 [SMILES: N#Cc1ccc(-c2ccc(-c3nc(-c4ccccc4)nc(-c4ccccc4-c4cc(-c5ccccc5-c5ccc(-c6cc7c8c(cccc8c6)-c6ccccc6-7)cc5)ccc4C#N)n3)cc2)cc1 ; N#Cc1ccc(-c2cccc(-c3nc(-c4ccccc4)nc(-c4cccc(-c5cc(-c6cccc(-c7ccc(-c8ccc9cccc%10c9c8-c8ccccc8-%10)cc7)c6)ccc5C#N)c4)n3)c2)cc1]

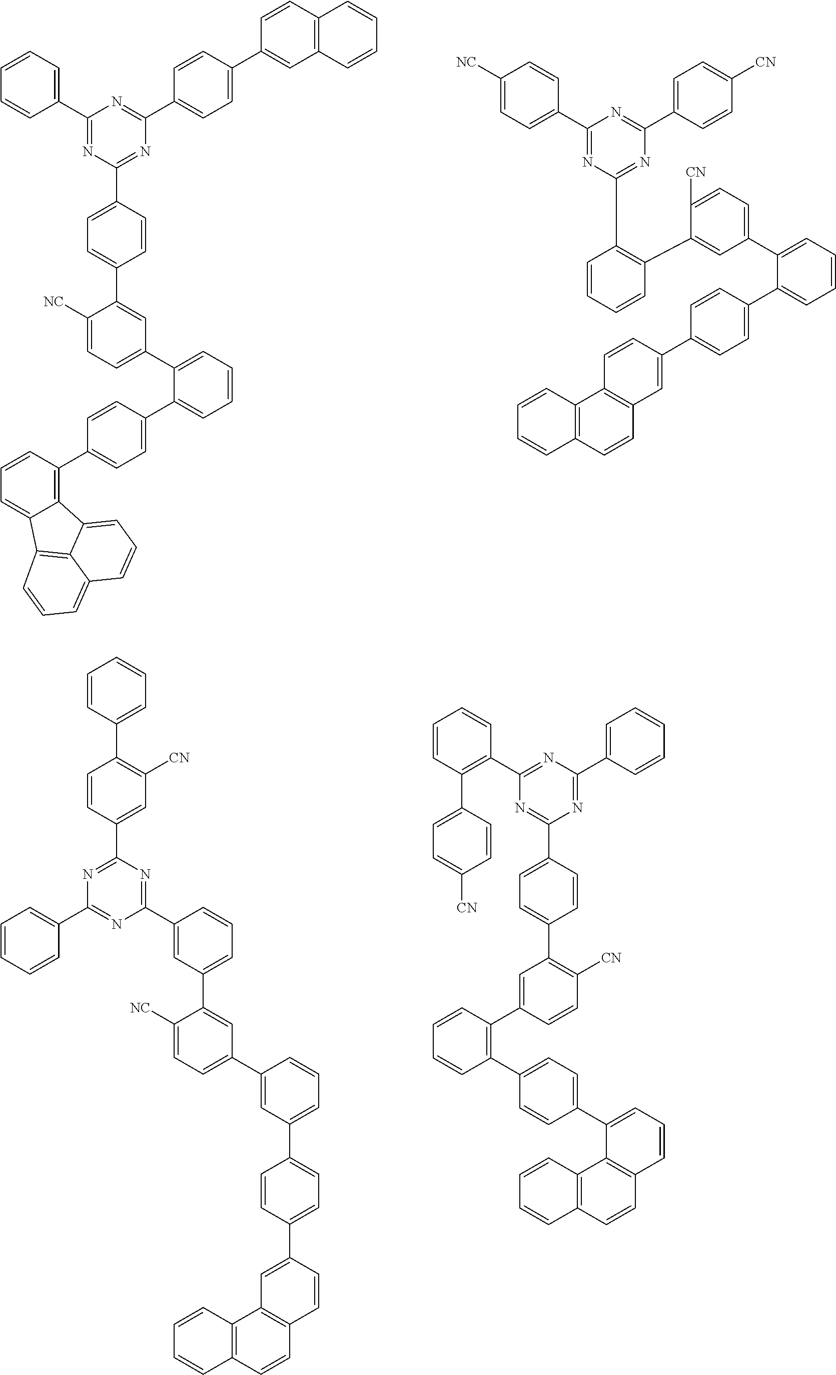 [SMILES: N#Cc1ccc(-c2ccccc2-c2ccc(-c3cccc4c3-c3cccc5cccc-4c35)cc2)cc1-c1ccc(-c2nc(-c3ccccc3)nc(-c3ccc(-c4ccc5ccccc5c4)cc3)n2)cc1 ; N#Cc1ccc(-c2nc(-c3ccc(C#N)cc3)nc(-c3ccccc3-c3cc(-c4ccccc4-c4ccc(-c5ccc6c(ccc7ccccc76)c5)cc4)ccc3C#N)n2)cc1 ; N#Cc1cc(-c2nc(-c3ccccc3)nc(-c3cccc(-c4cc(-c5cccc(-c6ccc(-c7ccc8ccc9ccccc9c8c7)cc6)c5)ccc4C#N)c3)n2)ccc1-c1ccccc1 ; N#Cc1ccc(-c2ccccc2-c2nc(-c3ccccc3)nc(-c3ccc(-c4cc(-c5ccccc5-c5ccc(-c6cccc7ccc8ccccc8c67)cc5)ccc4C#N)cc3)n2)cc1]

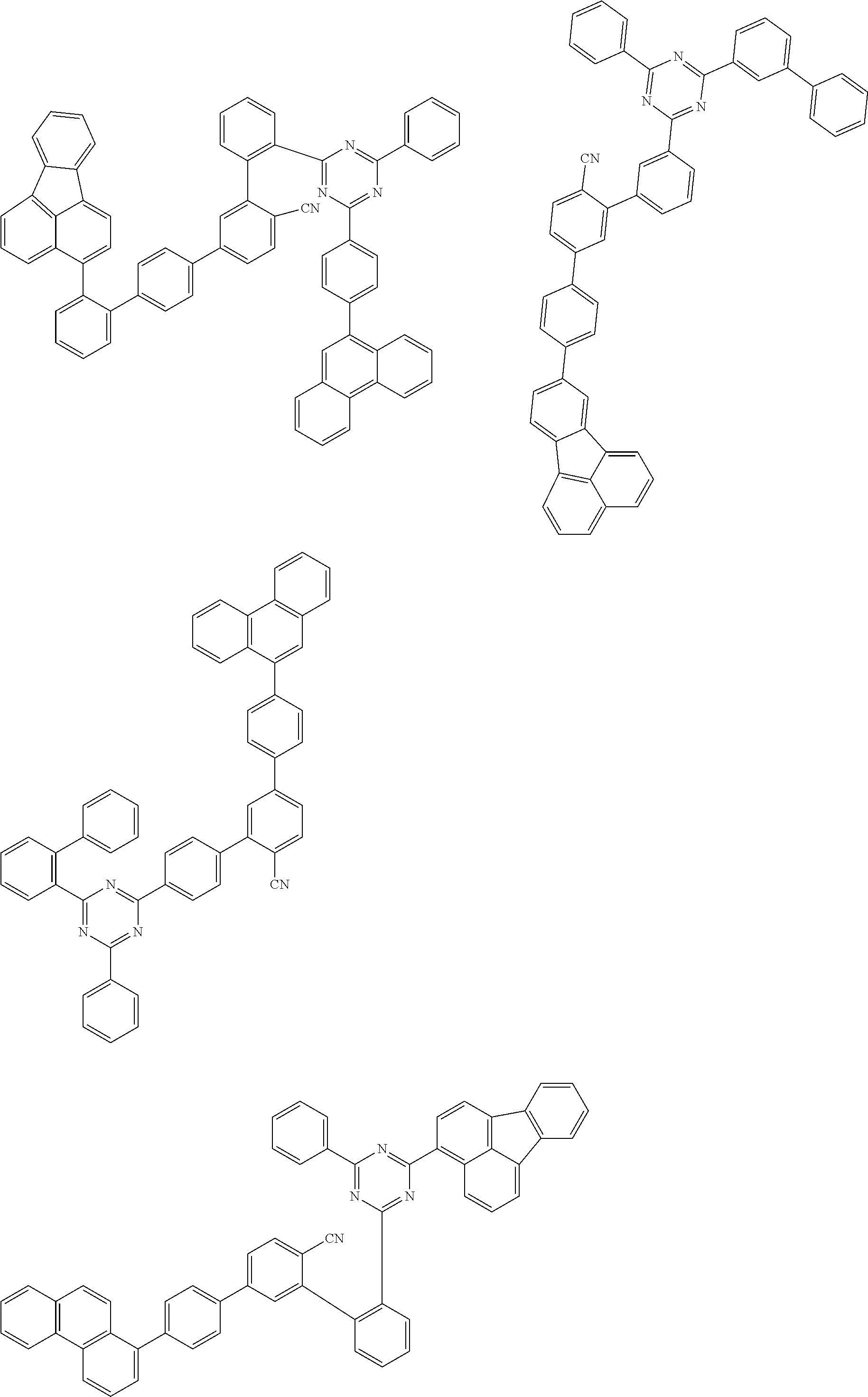 [SMILES: N#Cc1ccc(-c2ccc(-c3ccccc3-c3ccc4c5c(cccc35)-c3ccccc3-4)cc2)cc1-c1ccccc1-c1nc(-c2ccccc2)nc(-c2ccc(-c3cc4ccccc4c4ccccc34)cc2)n1 ; N#Cc1ccc(-c2ccc(-c3ccc4c(c3)-c3cccc5cccc-4c35)cc2)cc1-c1cccc(-c2nc(-c3ccccc3)nc(-c3cccc(-c4ccccc4)c3)n2)c1 ; N#Cc1ccc(-c2ccc(-c3cc4ccccc4c4ccccc34)cc2)cc1-c1ccc(-c2nc(-c3ccccc3)nc(-c3ccccc3-c3ccccc3)n2)cc1 ; N#Cc1ccc(-c2ccc(-c3cccc4c3ccc3ccccc34)cc2)cc1-c1ccccc1-c1nc(-c2ccccc2)nc(-c2ccc3c4c(cccc24)-c2ccccc2-3)n1]

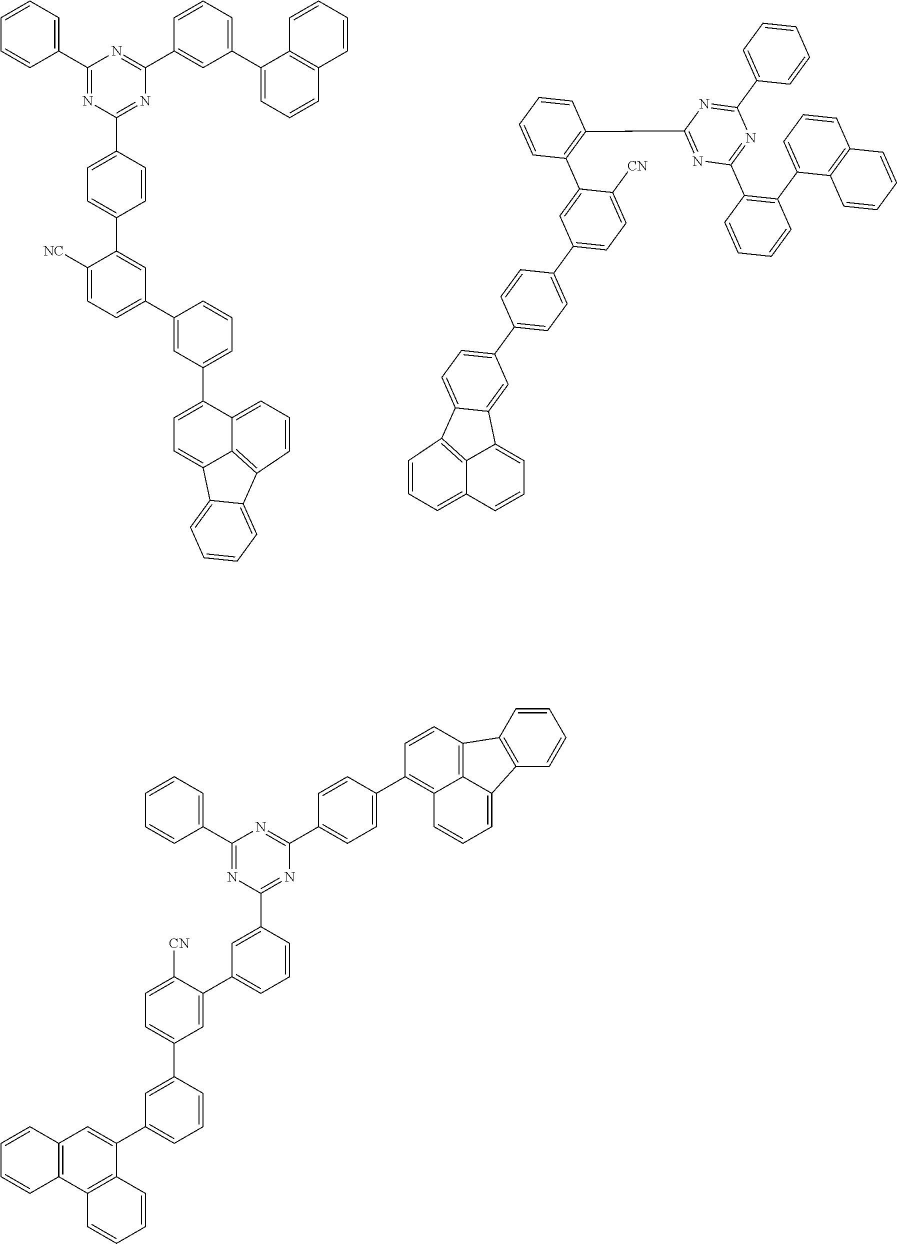

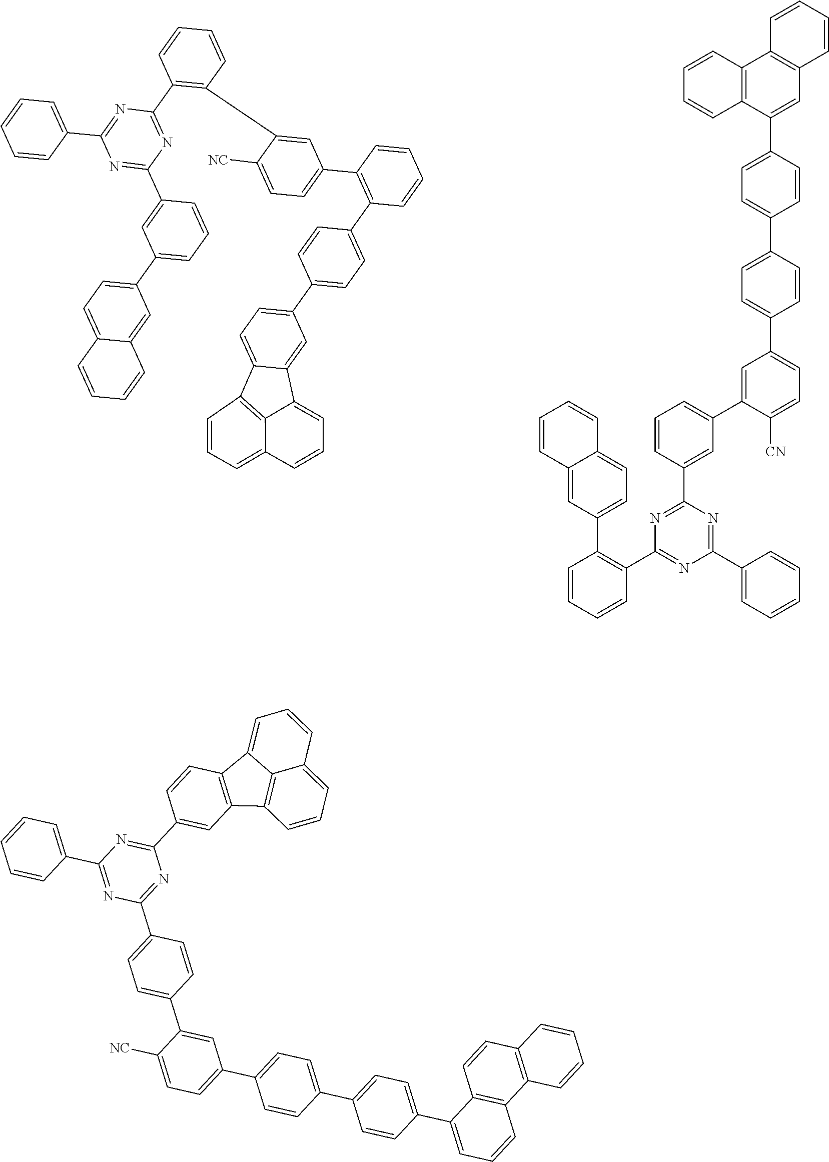 [SMILES: N#Cc1ccc(-c2ccccc2-c2ccc(-c3ccc4c(c3)-c3cccc5cccc-4c35)cc2)cc1-c1ccccc1-c1nc(-c2ccccc2)nc(-c2cccc(-c3ccc4ccccc4c3)c2)n1 ; N#Cc1ccc(-c2ccc(-c3ccc(-c4cc5ccccc5c5ccccc45)cc3)cc2)cc1-c1cccc(-c2nc(-c3ccccc3)nc(-c3ccccc3-c3ccc4ccccc4c3)n2)c1 ; N#Cc1ccc(-c2ccc(-c3ccc(-c4cccc5c4ccc4ccccc45)cc3)cc2)cc1-c1ccc(-c2nc(-c3ccccc3)nc(-c3ccc4c(c3)-c3cccc5cccc-4c35)n2)cc1]

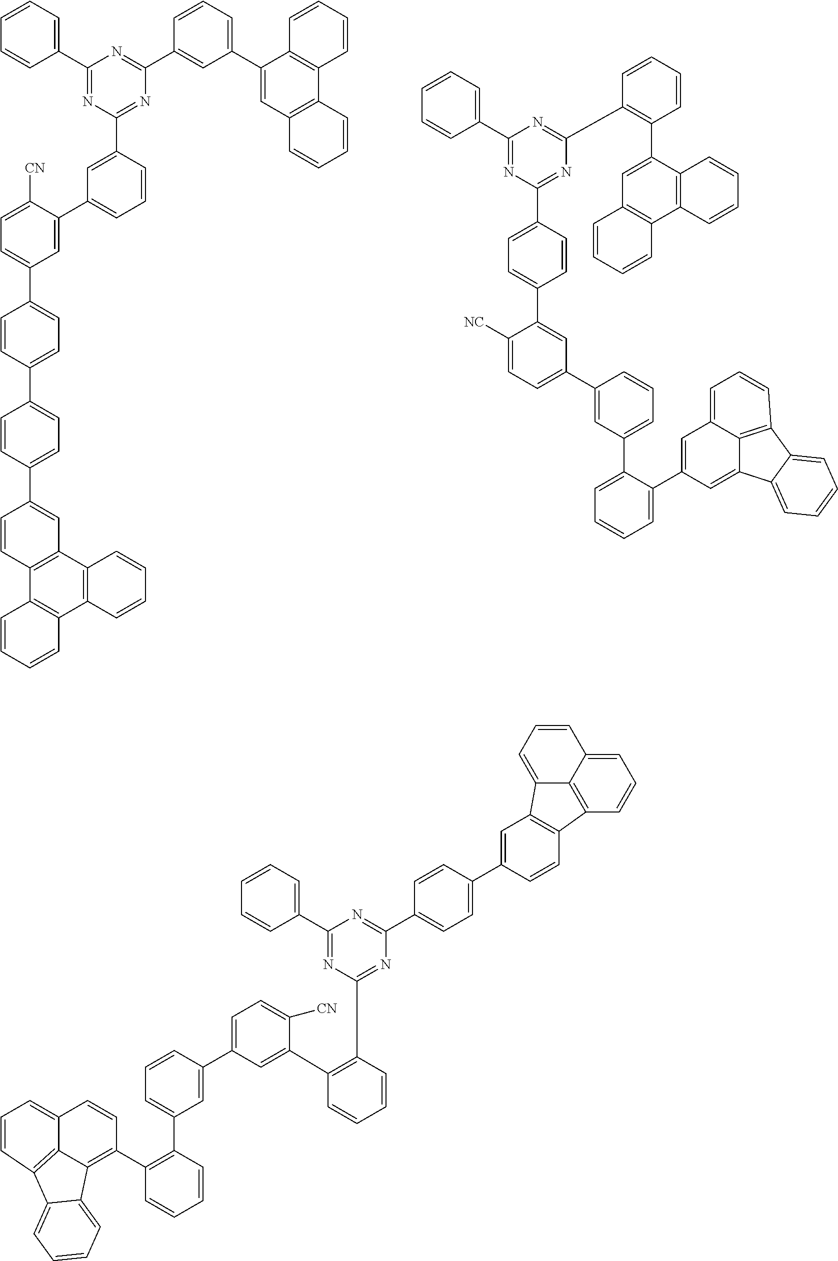 [SMILES: N#Cc1ccc(-c2ccc(-c3ccc(-c4ccc5c6ccccc6c6ccccc6c5c4)cc3)cc2)cc1-c1cccc(-c2nc(-c3ccccc3)nc(-c3cccc(-c4cc5ccccc5c5ccccc45)c3)n2)c1 ; N#Cc1ccc(-c2cccc(-c3ccccc3-c3cc4c5c(cccc5c3)-c3ccccc3-4)c2)cc1-c1ccc(-c2nc(-c3ccccc3)nc(-c3ccccc3-c3cc4ccccc4c4ccccc34)n2)cc1 ; N#Cc1ccc(-c2cccc(-c3ccccc3-c3ccc4cccc5c4c3-c3ccccc3-5)c2)cc1-c1ccccc1-c1nc(-c2ccccc2)nc(-c2ccc(-c3ccc4c(c3)-c3cccc5cccc-4c35)cc2)n1]

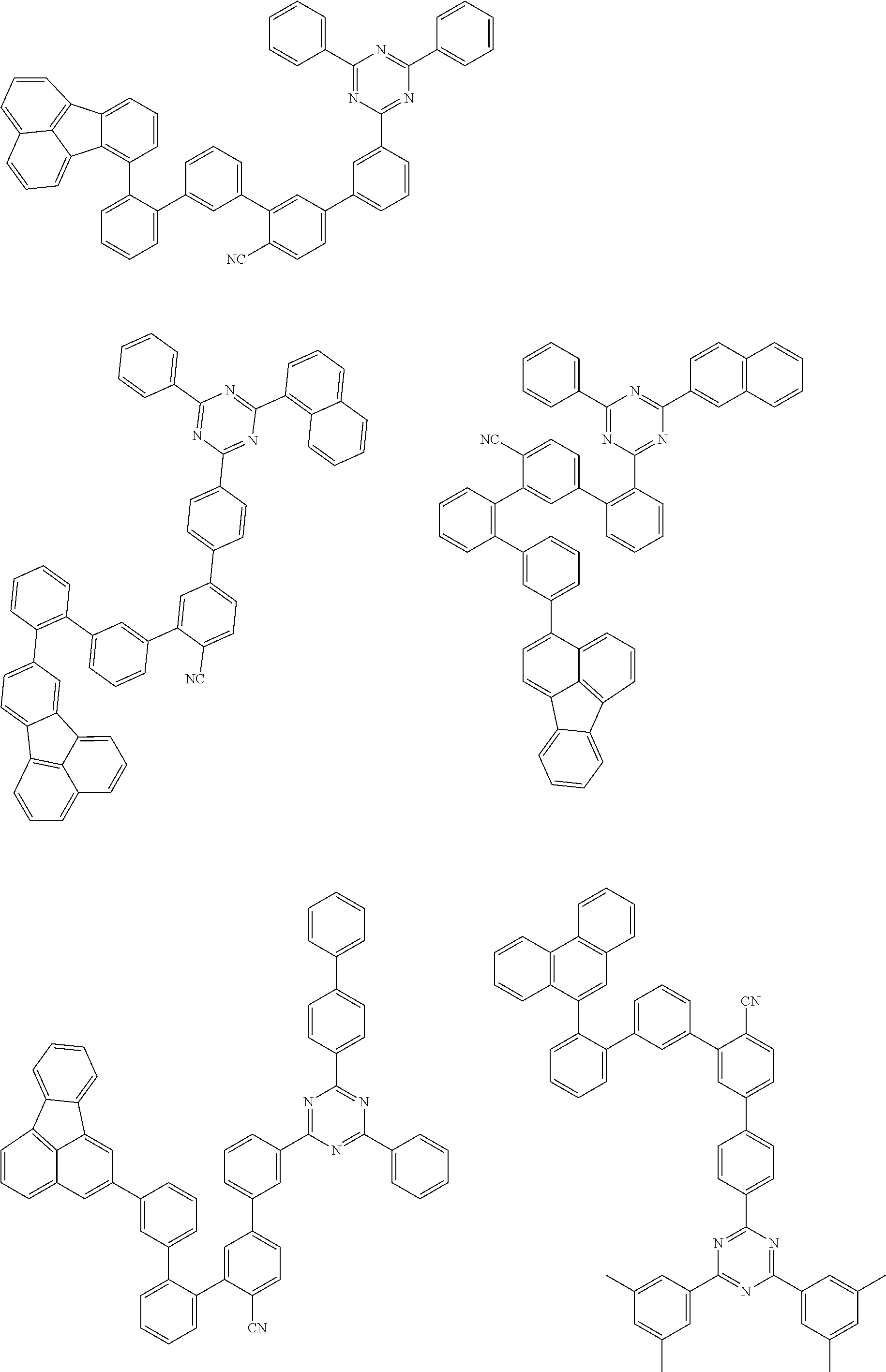

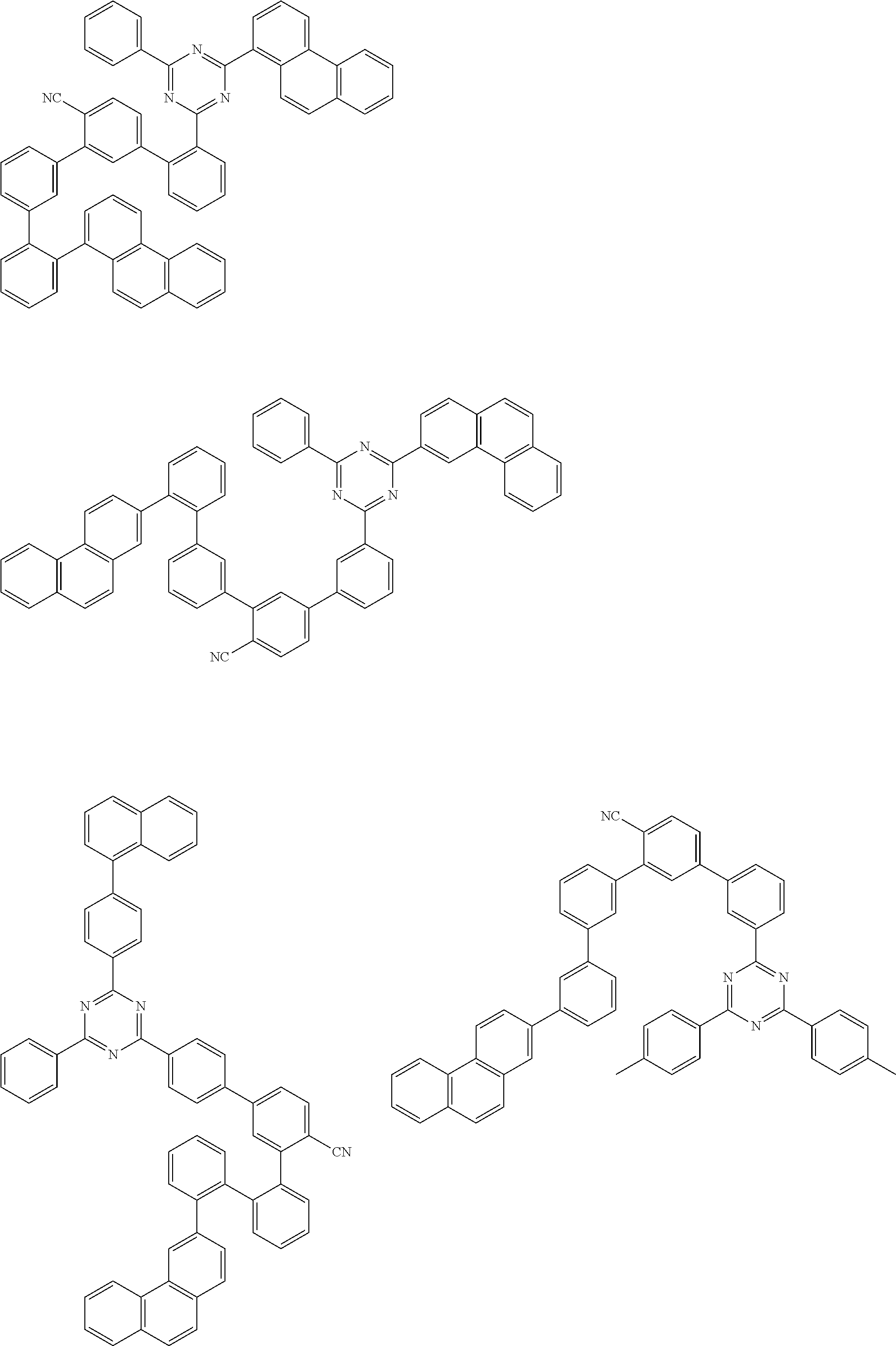 [SMILES: N#Cc1ccc(-c2ccccc2-c2nc(-c3ccccc3)nc(-c3cccc4c3ccc3ccccc34)n2)cc1-c1cccc(-c2ccccc2-c2cccc3c2ccc2ccccc23)c1 ; N#Cc1ccc(-c2cccc(-c3nc(-c4ccccc4)nc(-c4ccc5ccc6ccccc6c5c4)n3)c2)cc1-c1cccc(-c2ccccc2-c2ccc3c(ccc4ccccc43)c2)c1 ; N#Cc1ccc(-c2ccc(-c3nc(-c4ccccc4)nc(-c4ccc(-c5cccc6ccccc56)cc4)n3)cc2)cc1-c1ccccc1-c1ccccc1-c1ccc2ccc3ccccc3c2c1 ; Cc1ccc(-c2nc(-c3ccc(C)cc3)nc(-c3cccc(-c4ccc(C#N)c(-c5cccc(-c6cccc(-c7ccc8c(ccc9ccccc98)c7)c6)c5)c4)c3)n2)cc1]

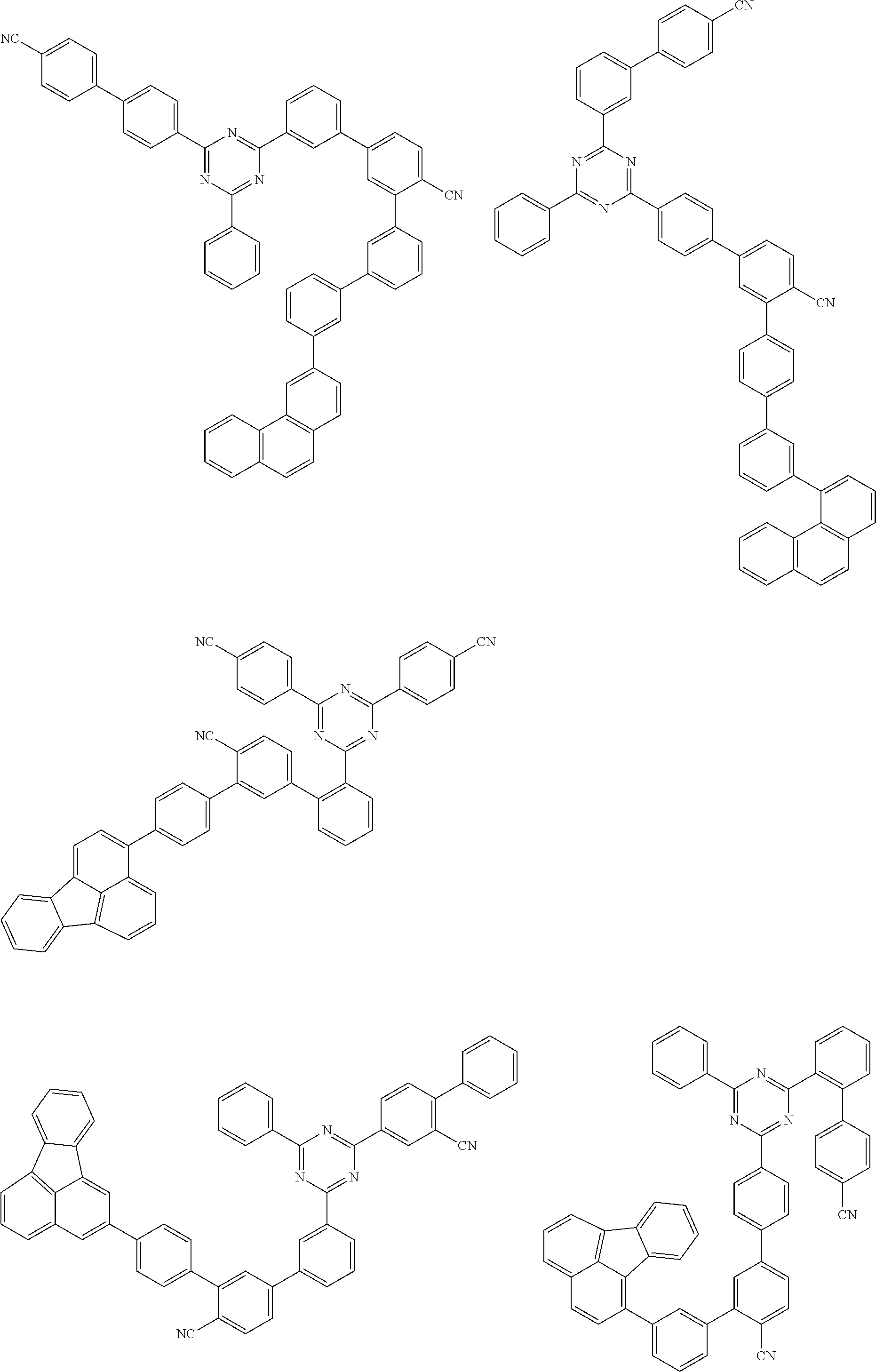

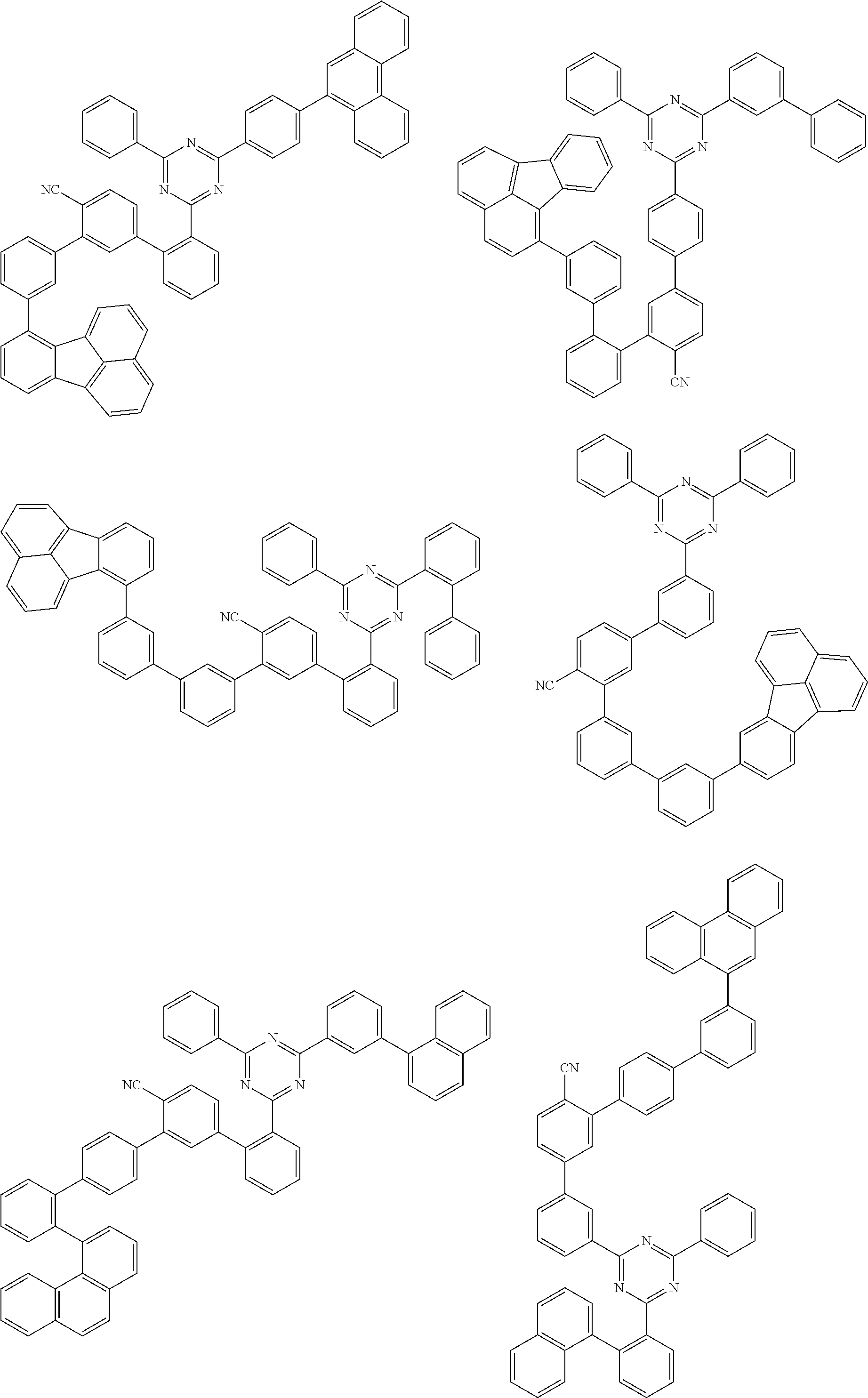

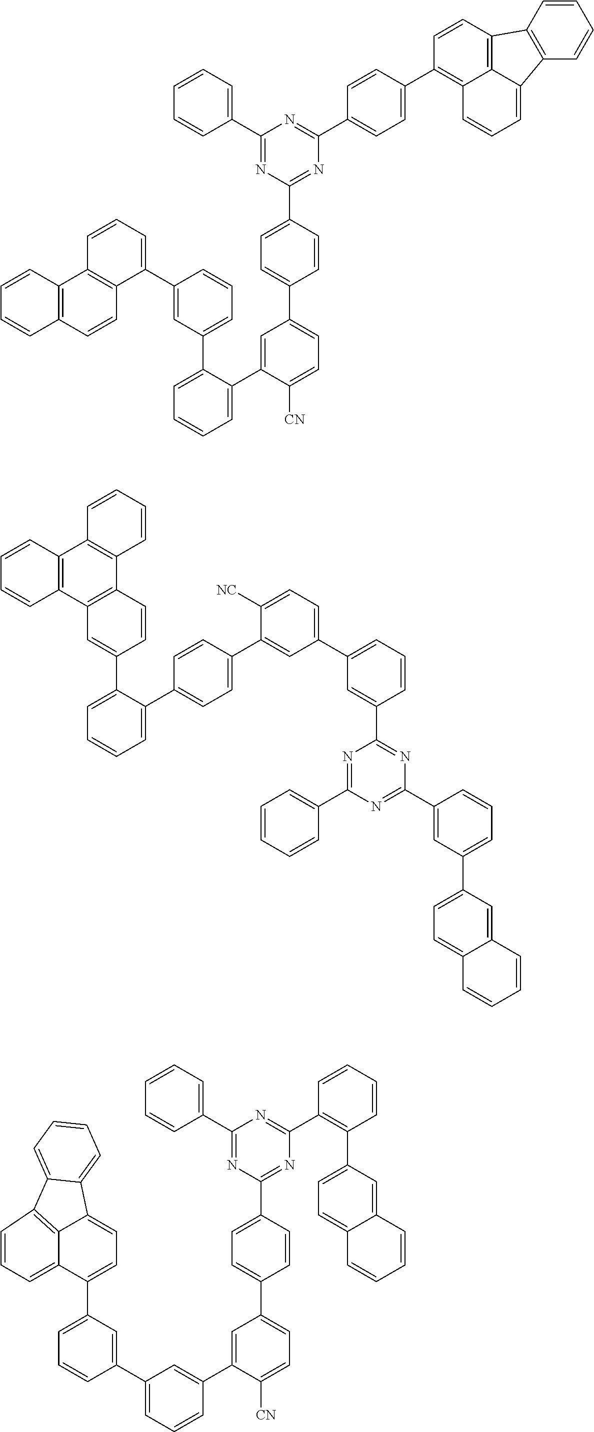 [SMILES: N#Cc1ccc(-c2ccc(-c3nc(-c4ccccc4)nc(-c4ccc(-c5ccc6c7c(cccc57)-c5ccccc5-6)cc4)n3)cc2)cc1-c1ccccc1-c1cccc(-c2cccc3c2ccc2ccccc23)c1 ; N#Cc1ccc(-c2cccc(-c3nc(-c4ccccc4)nc(-c4cccc(-c5ccc6ccccc6c5)c4)n3)c2)cc1-c1ccc(-c2ccccc2-c2ccc3c4ccccc4c4ccccc4c3c2)cc1 ; N#Cc1ccc(-c2ccc(-c3nc(-c4ccccc4)nc(-c4ccccc4-c4ccc5ccccc5c4)n3)cc2)cc1-c1cccc(-c2cccc(-c3ccc4c5c(cccc35)-c3ccccc3-4)c2)c1]

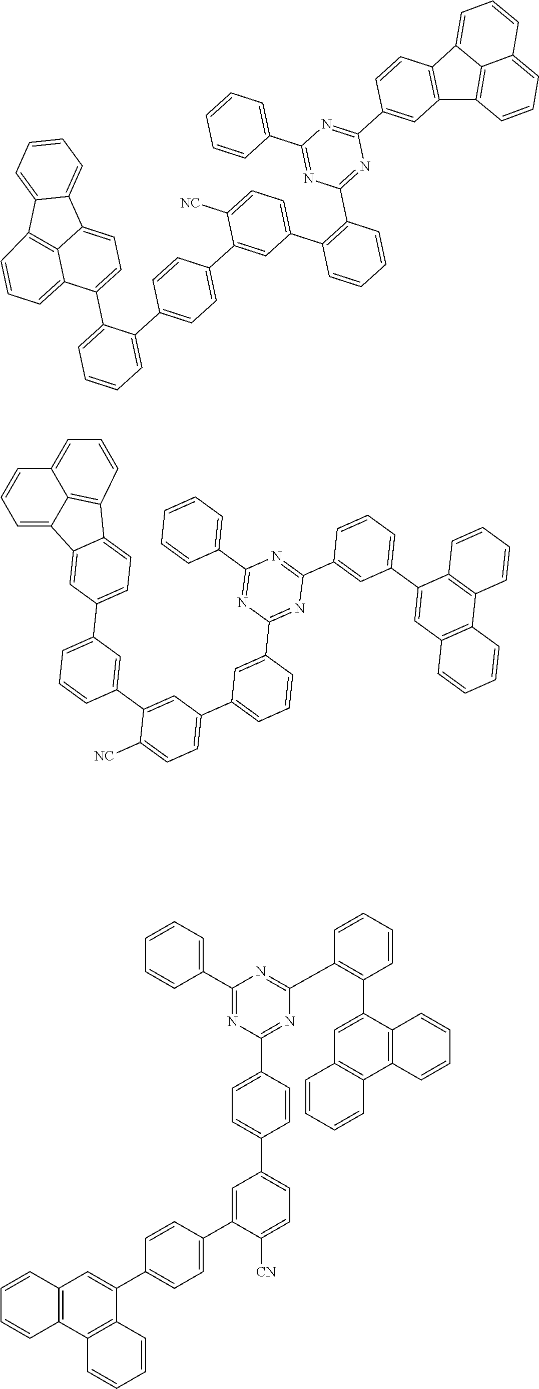 [SMILES: N#Cc1ccc(-c2ccccc2-c2nc(-c3ccccc3)nc(-c3ccc4c(c3)-c3cccc5cccc-4c35)n2)cc1-c1ccc(-c2ccccc2-c2ccc3c4c(cccc24)-c2ccccc2-3)cc1 ; N#Cc1ccc(-c2cccc(-c3nc(-c4ccccc4)nc(-c4cccc(-c5cc6ccccc6c6ccccc56)c4)n3)c2)cc1-c1cccc(-c2ccc3c(c2)-c2cccc4cccc-3c24)c1 ; N#Cc1ccc(-c2ccc(-c3nc(-c4ccccc4)nc(-c4ccccc4-c4cc5ccccc5c5ccccc45)n3)cc2)cc1-c1ccc(-c2cc3ccccc3c3ccccc23)cc1]

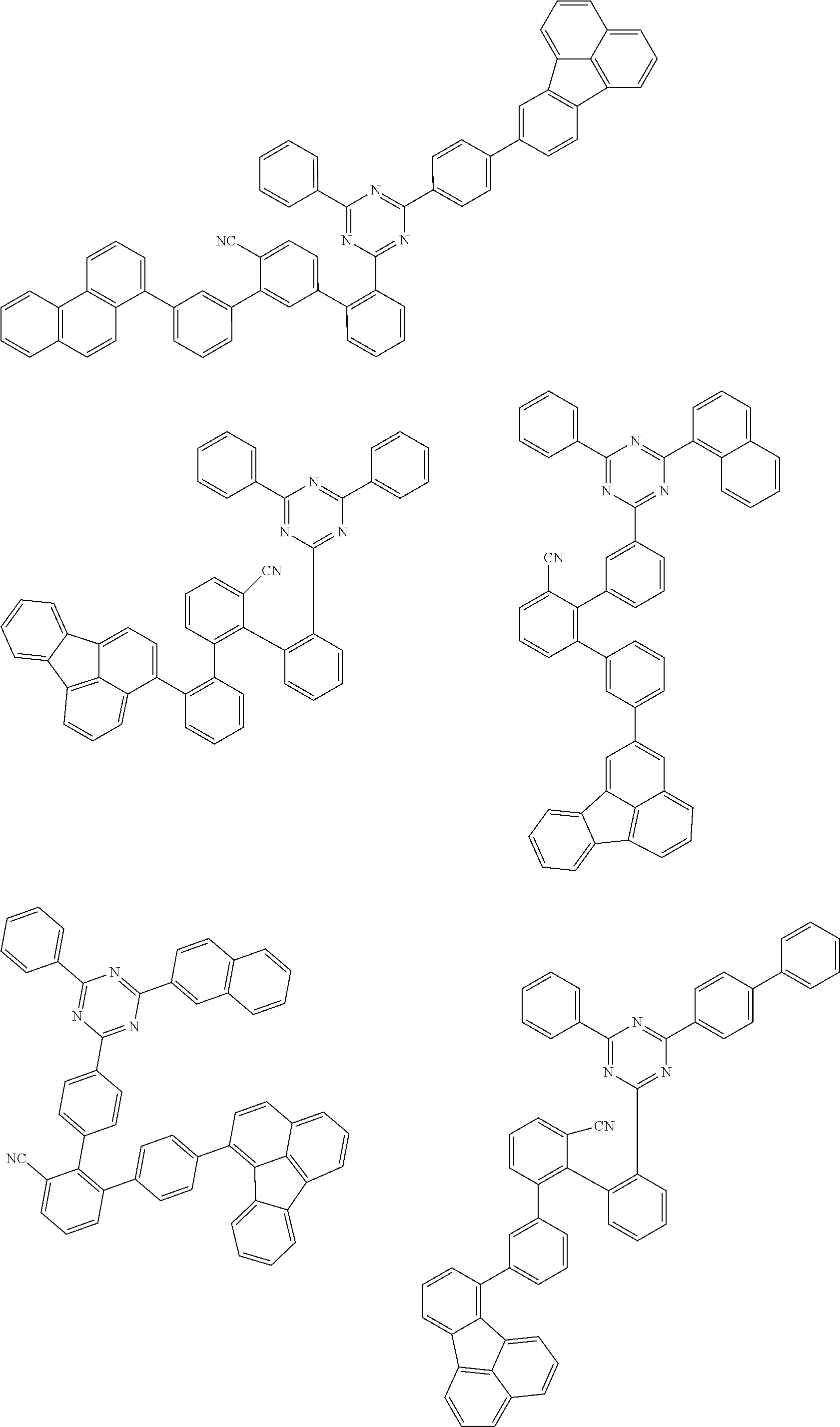

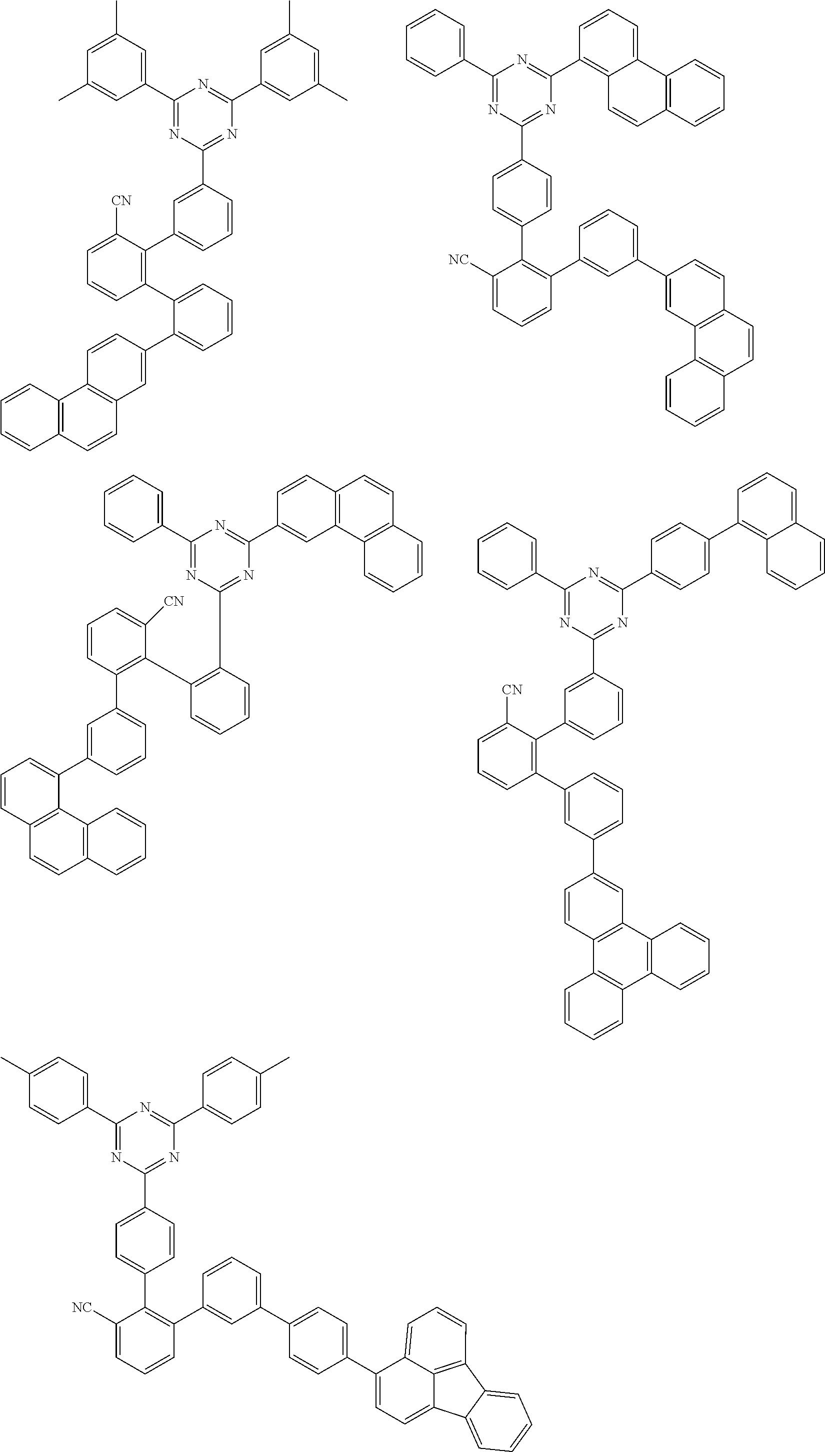

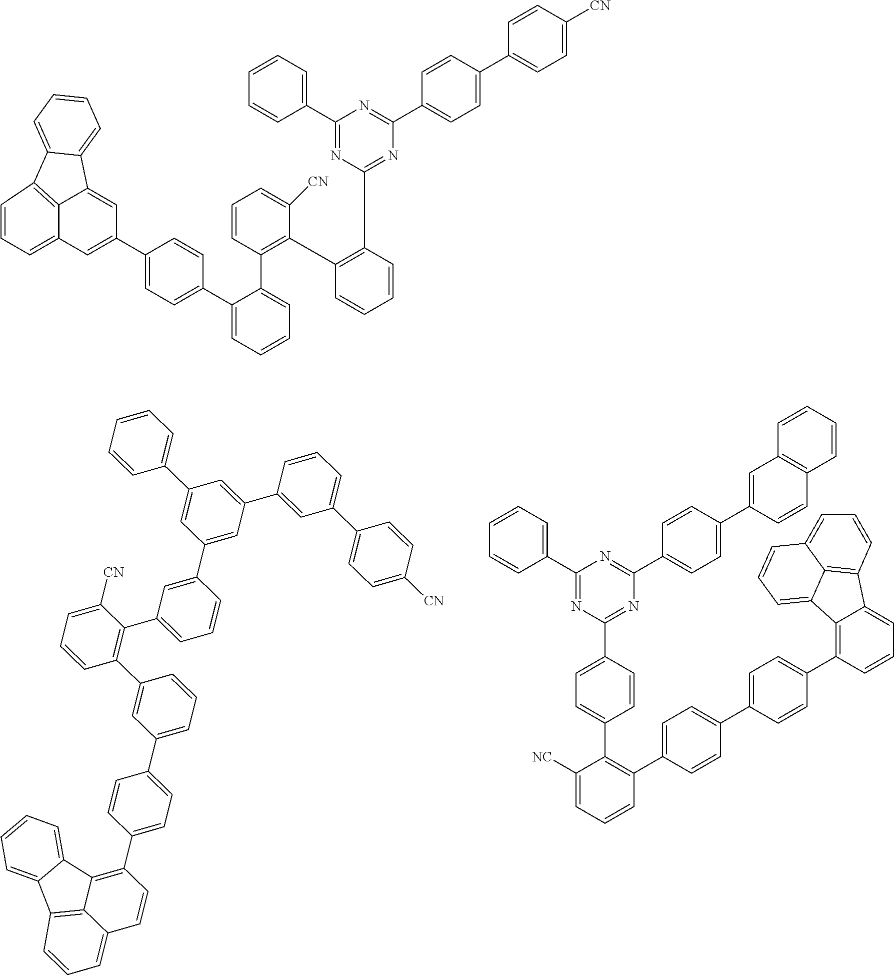 [SMILES: N#Cc1ccc(-c2ccc(-c3nc(-c4ccccc4)nc(-c4ccccc4-c4c(C#N)cccc4-c4ccccc4-c4ccc(-c5cc6c7c(cccc7c5)-c5ccccc5-6)cc4)n3)cc2)cc1 ; N#Cc1ccc(-c2cccc(-c3cc(-c4ccccc4)cc(-c4cccc(-c5c(C#N)cccc5-c5cccc(-c6ccc(-c7ccc8cccc9c8c7-c7ccccc7-9)cc6)c5)c4)c3)c2)cc1 ; N#Cc1cccc(-c2ccc(-c3ccc(-c4cccc5c4-c4cccc6cccc-5c46)cc3)cc2)c1-c1ccc(-c2nc(-c3ccccc3)nc(-c3ccc(-c4ccc5ccccc5c4)cc3)n2)cc1]

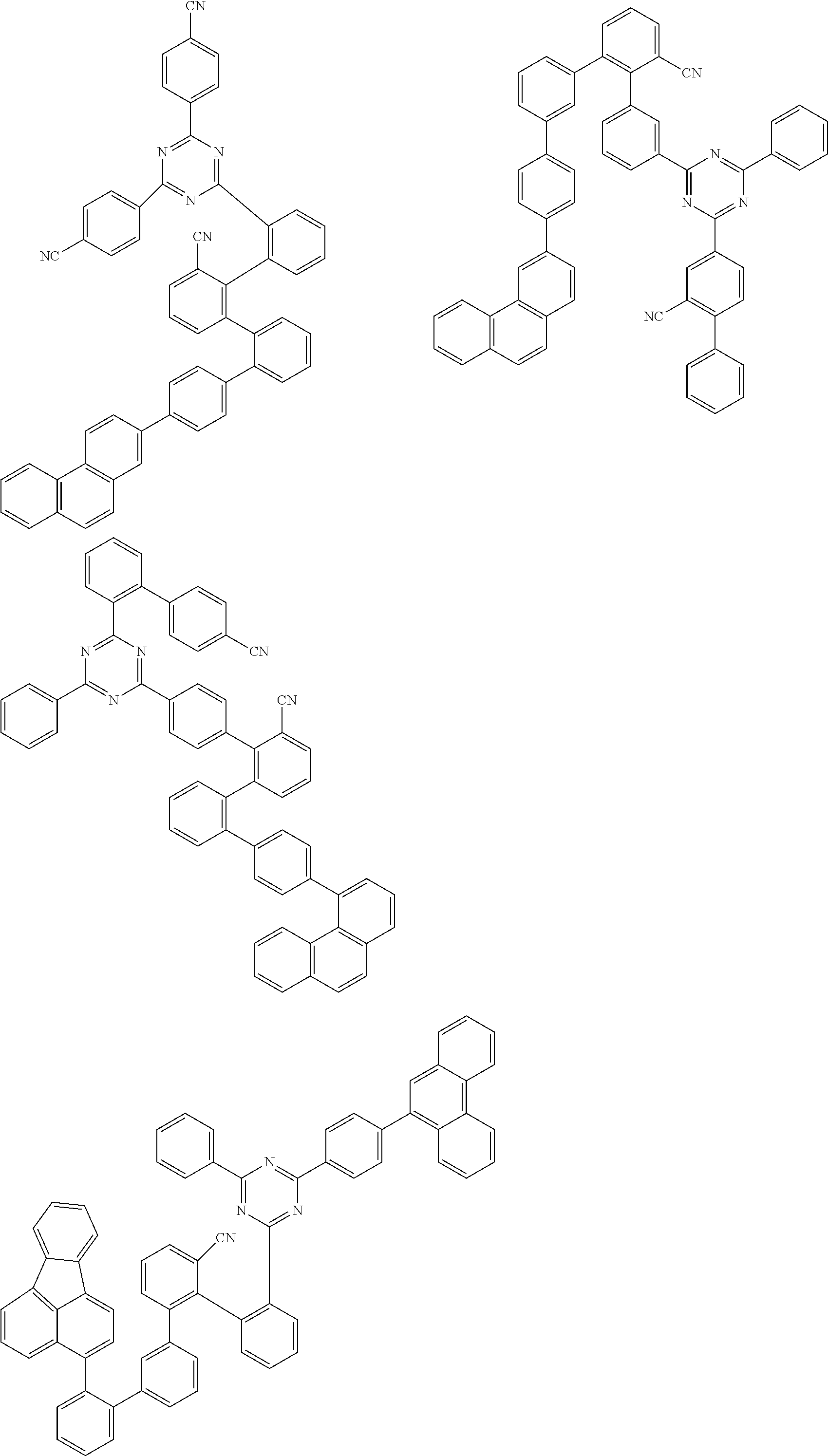

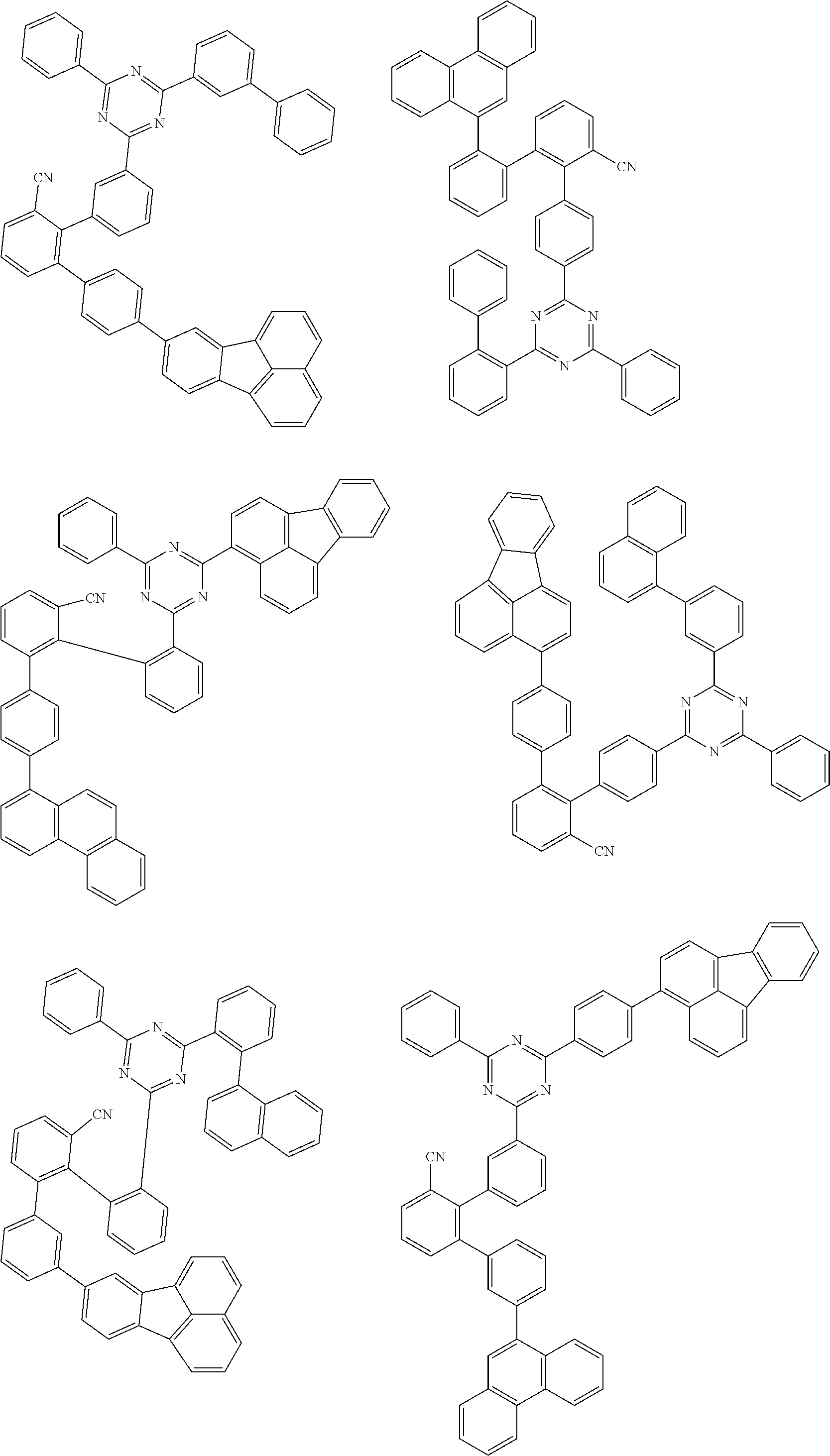

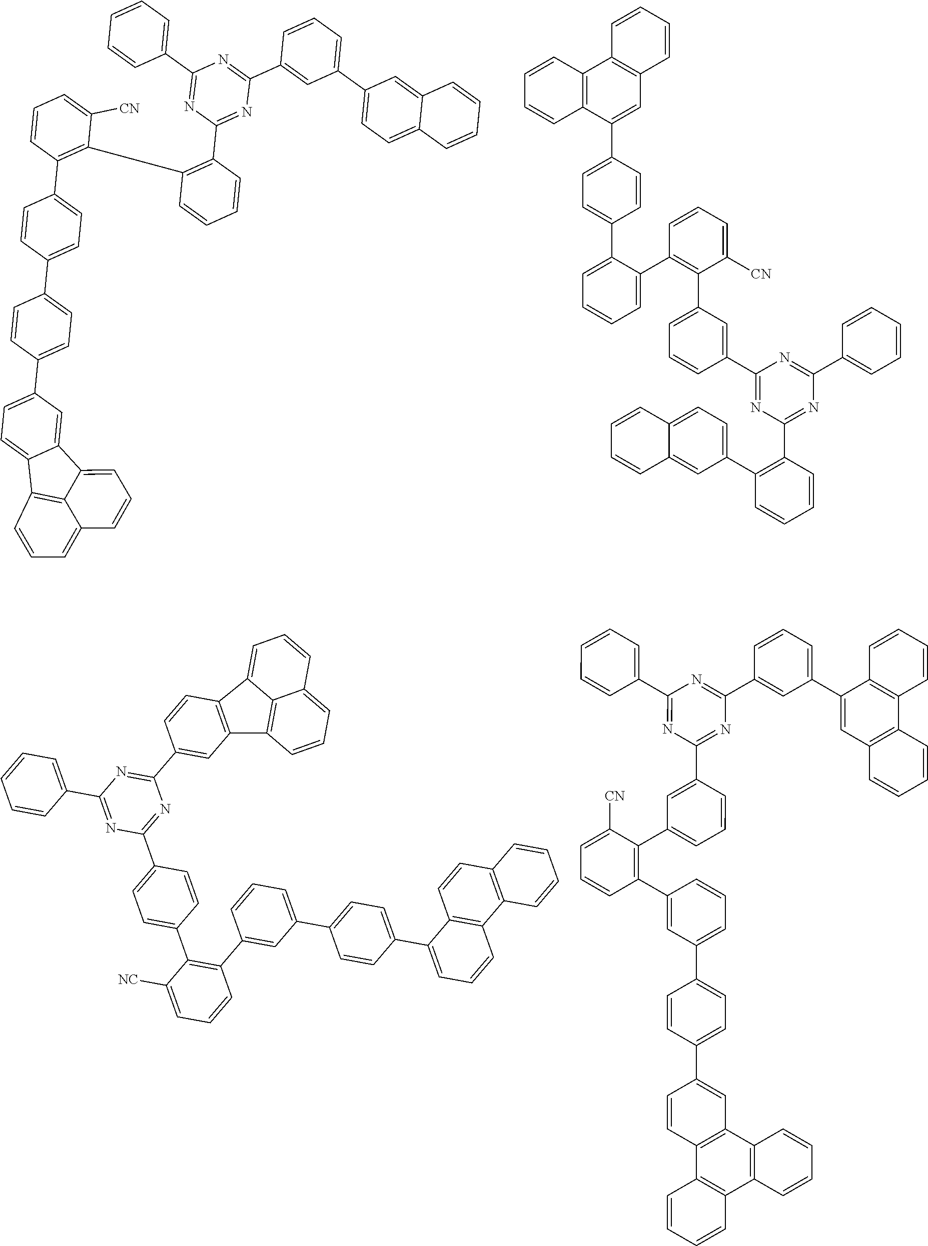

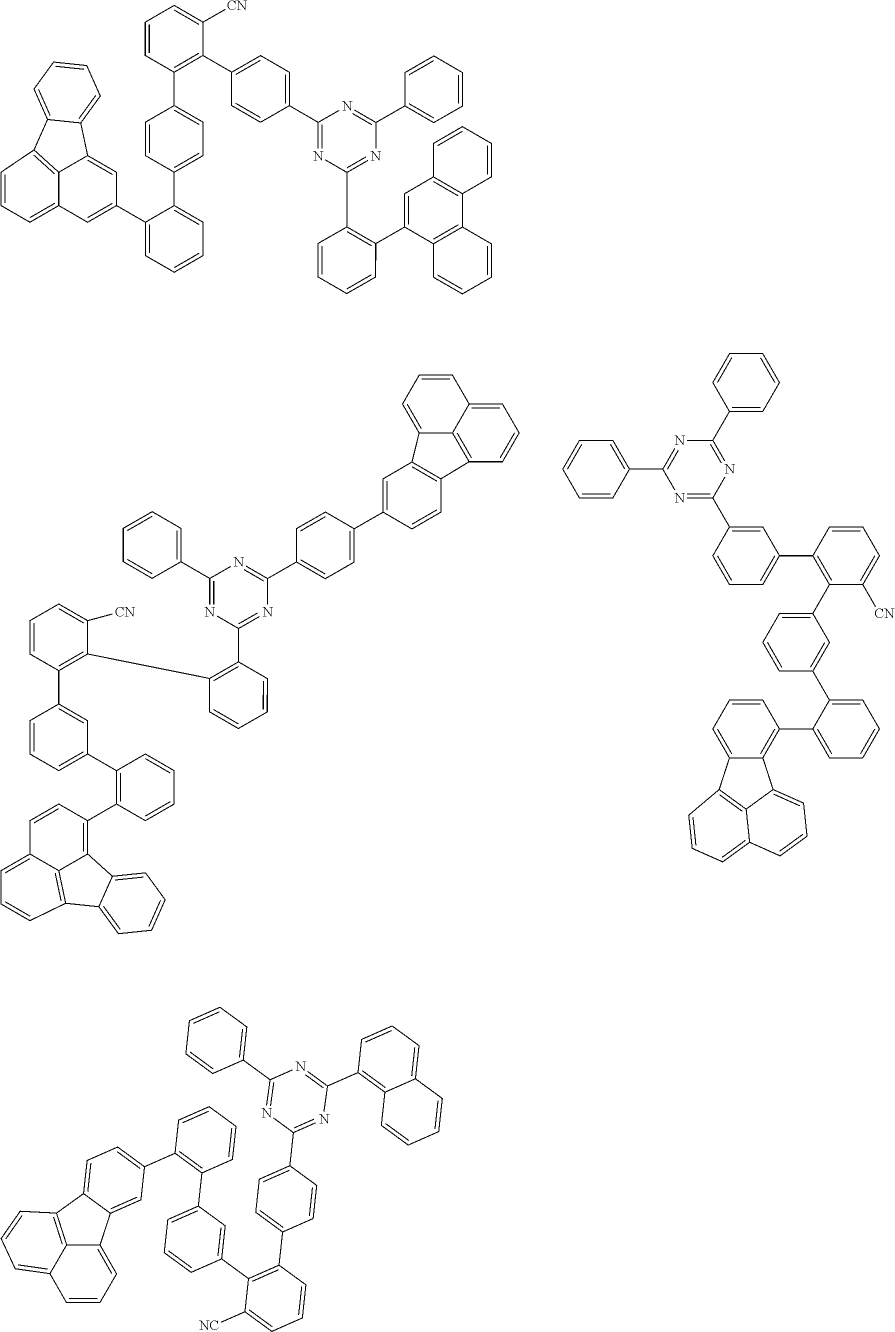 [SMILES: N#Cc1cccc(-c2ccc(-c3ccccc3-c3cc4c5c(cccc5c3)-c3ccccc3-4)cc2)c1-c1ccc(-c2nc(-c3ccccc3)nc(-c3ccccc3-c3cc4ccccc4c4ccccc34)n2)cc1 ; N#Cc1cccc(-c2cccc(-c3ccccc3-c3ccc4cccc5c4c3-c3ccccc3-5)c2)c1-c1ccccc1-c1nc(-c2ccccc2)nc(-c2ccc(-c3ccc4c(c3)-c3cccc5cccc-4c35)cc2)n1 ; N#Cc1cccc(-c2cccc(-c3nc(-c4ccccc4)nc(-c4ccccc4)n3)c2)c1-c1cccc(-c2ccccc2-c2cccc3c2-c2cccc4cccc-3c24)c1 ; N#Cc1cccc(-c2ccc(-c3nc(-c4ccccc4)nc(-c4cccc5ccccc45)n3)cc2)c1-c1cccc(-c2ccccc2-c2ccc3c(c2)-c2cccc4cccc-3c24)c1]

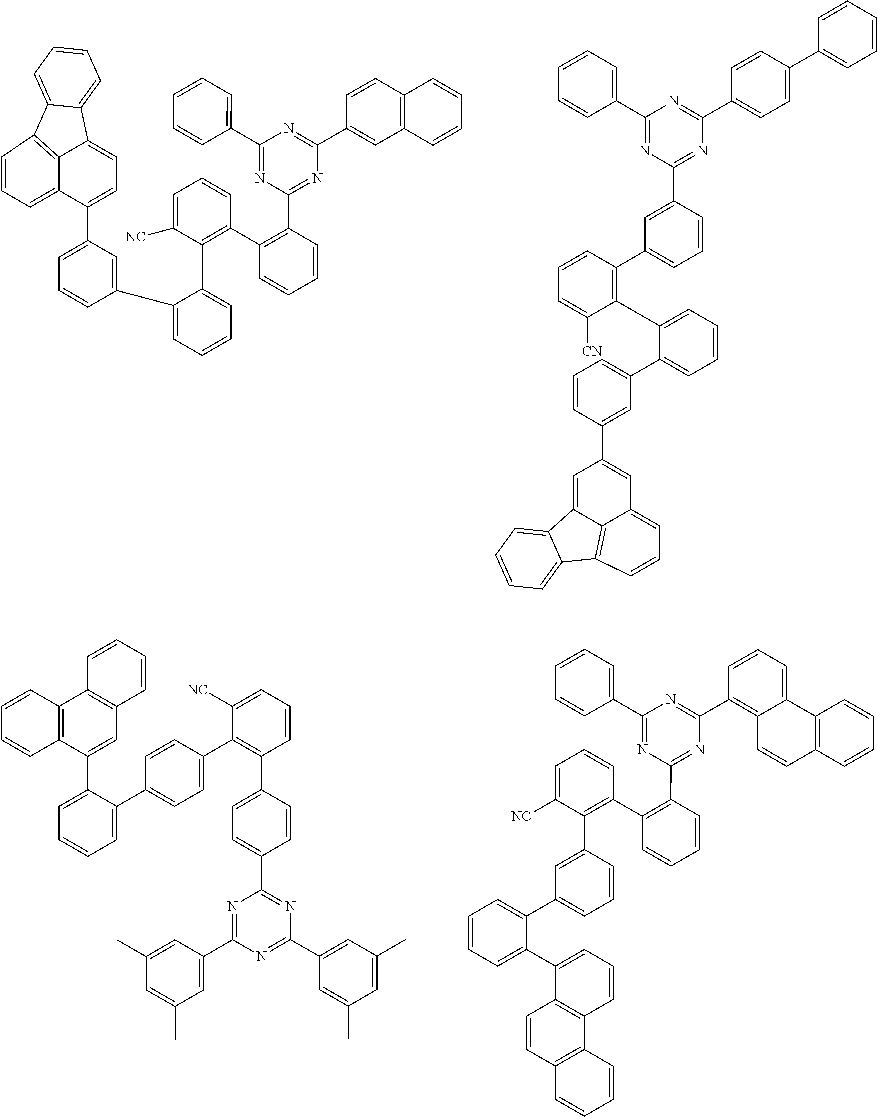 [SMILES: N#Cc1cccc(-c2ccccc2-c2nc(-c3ccccc3)nc(-c3ccc4ccccc4c3)n2)c1-c1ccccc1-c1cccc(-c2ccc3c4c(cccc24)-c2ccccc2-3)c1 ; N#Cc1cccc(-c2cccc(-c3nc(-c4ccccc4)nc(-c4ccc(-c5ccccc5)cc4)n3)c2)c1-c1ccccc1-c1cccc(-c2cc3c4c(cccc4c2)-c2ccccc2-3)c1 ; Cc1cc(C)cc(-c2nc(-c3ccc(-c4cccc(C#N)c4-c4ccc(-c5ccccc5-c5cc6ccccc6c6ccccc56)cc4)cc3)nc(-c3cc(C)cc(C)c3)n2)c1 ; N#Cc1cccc(-c2ccccc2-c2nc(-c3ccccc3)nc(-c3cccc4c3ccc3ccccc34)n2)c1-c1cccc(-c2ccccc2-c2cccc3c2ccc2ccccc23)c1]

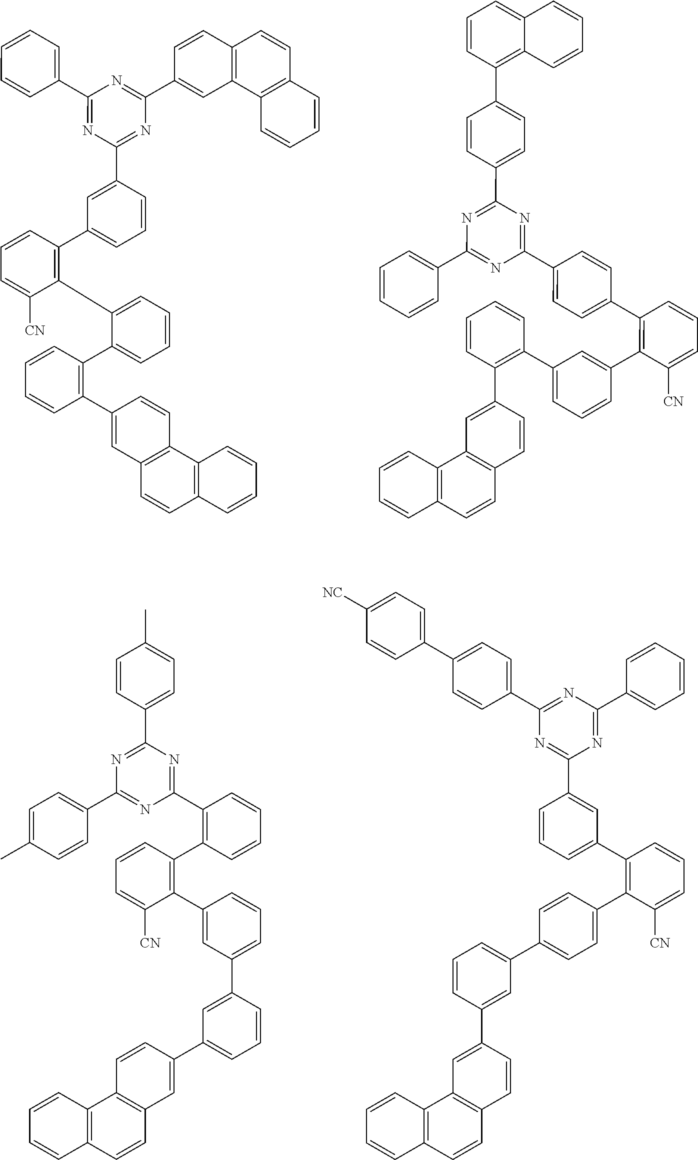 [SMILES: N#Cc1cccc(-c2cccc(-c3nc(-c4ccccc4)nc(-c4ccc5ccc6ccccc6c5c4)n3)c2)c1-c1ccccc1-c1ccccc1-c1ccc2c(ccc3ccccc32)c1 ; N#Cc1cccc(-c2ccc(-c3nc(-c4ccccc4)nc(-c4ccc(-c5cccc6ccccc56)cc4)n3)cc2)c1-c1cccc(-c2ccccc2-c2ccc3ccc4ccccc4c3c2)c1 ; Cc1ccc(-c2nc(-c3ccc(C)cc3)nc(-c3ccccc3-c3cccc(C#N)c3-c3cccc(-c4cccc(-c5ccc6c(ccc7ccccc76)c5)c4)c3)n2)cc1 ; N#Cc1ccc(-c2ccc(-c3nc(-c4ccccc4)nc(-c4cccc(-c5cccc(C#N)c5-c5ccc(-c6cccc(-c7ccc8ccc9ccccc9c8c7)c6)cc5)c4)n3)cc2)cc1]

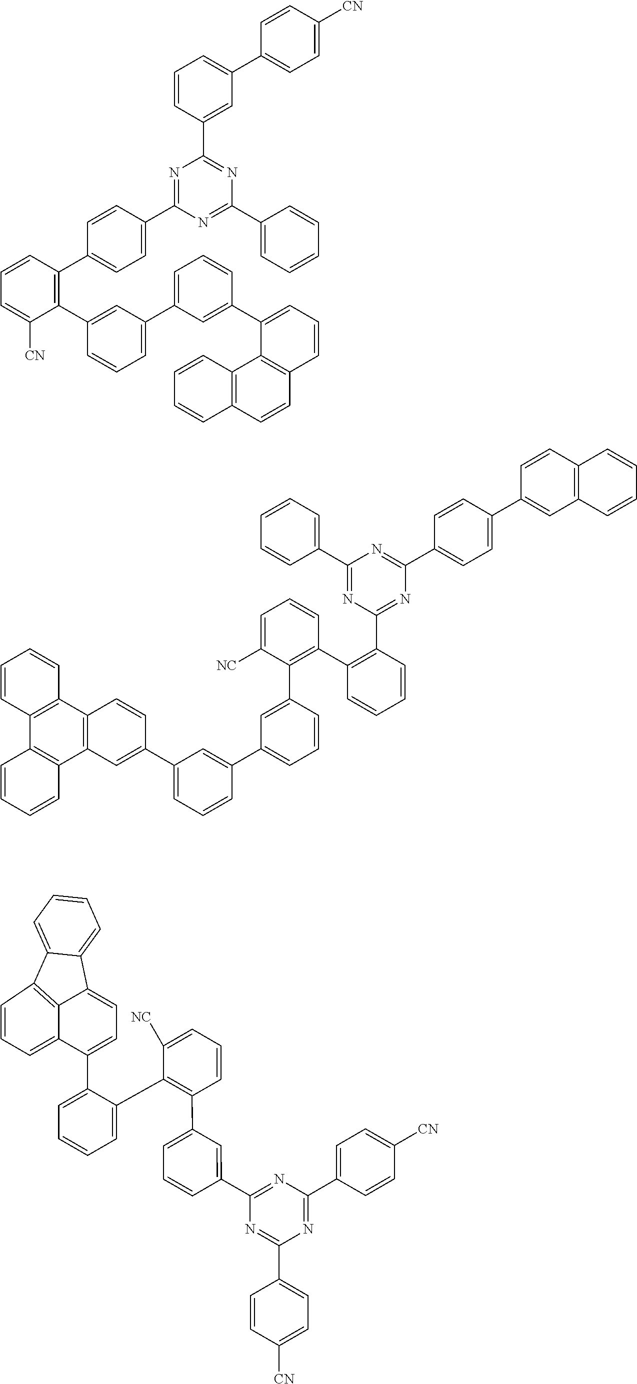 [SMILES: N#Cc1ccc(-c2cccc(-c3nc(-c4ccccc4)nc(-c4ccc(-c5cccc(C#N)c5-c5cccc(-c6cccc(-c7cccc8ccc9ccccc9c78)c6)c5)cc4)n3)c2)cc1 ; N#Cc1cccc(-c2ccccc2-c2nc(-c3ccccc3)nc(-c3ccc(-c4ccc5ccccc5c4)cc3)n2)c1-c1cccc(-c2cccc(-c3ccc4c5ccccc5c5ccccc5c4c3)c2)c1 ; N#Cc1ccc(-c2nc(-c3ccc(C#N)cc3)nc(-c3cccc(-c4cccc(C#N)c4-c4ccccc4-c4ccc5c6c(cccc46)-c4ccccc4-5)c3)n2)cc1]

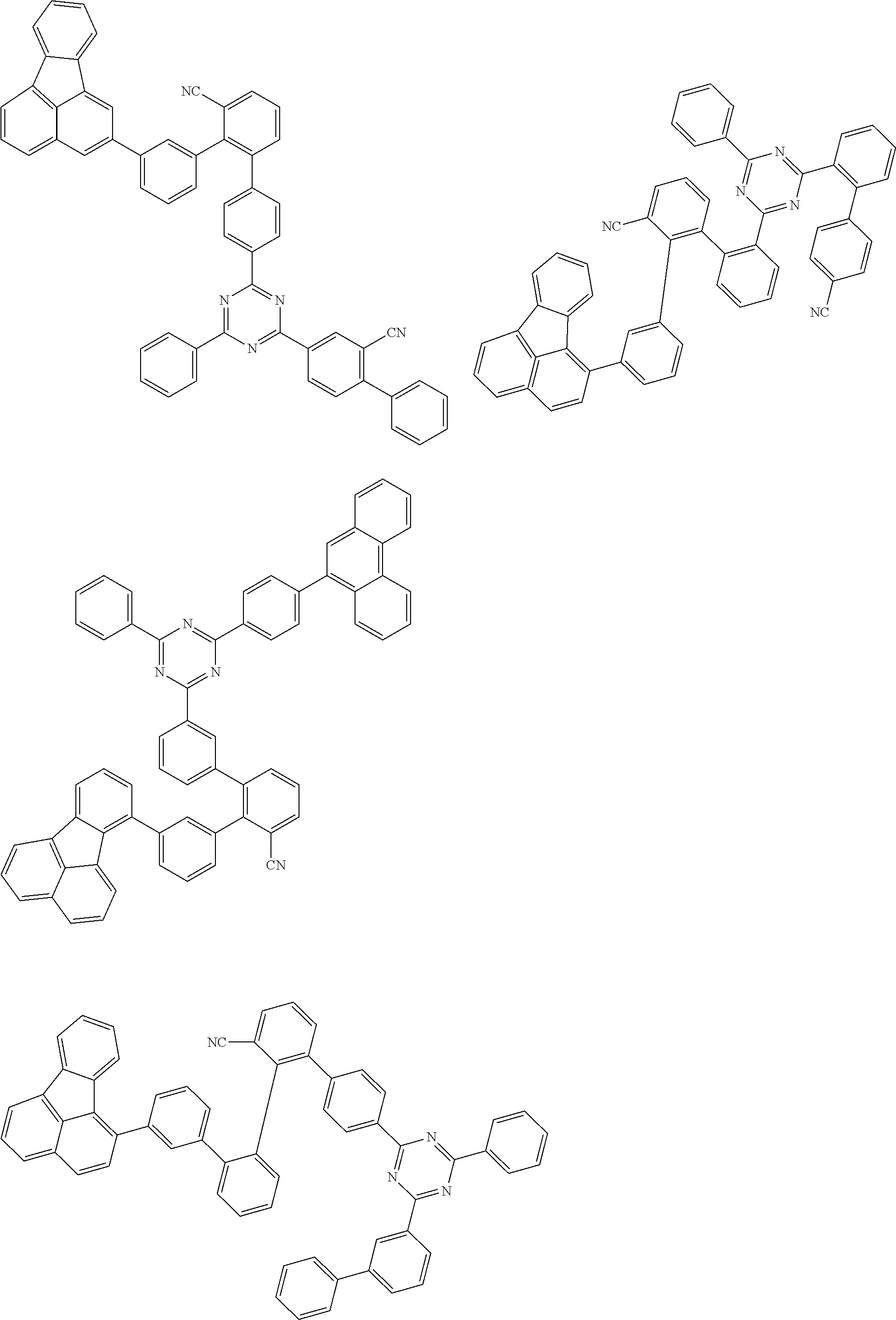

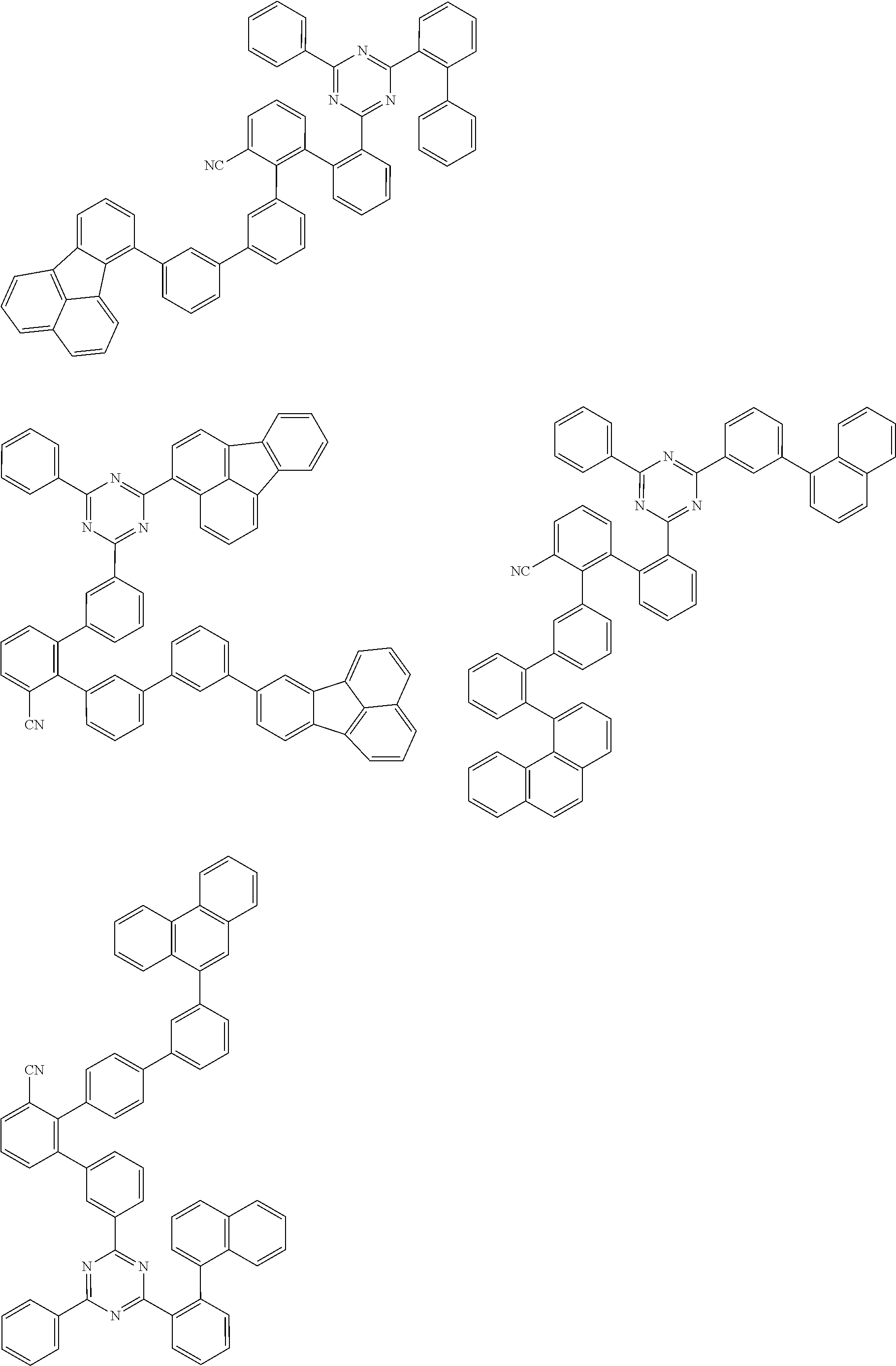 [SMILES: N#Cc1cccc(-c2ccccc2-c2nc(-c3ccccc3)nc(-c3ccccc3-c3ccccc3)n2)c1-c1cccc(-c2cccc(-c3cccc4c3-c3cccc5cccc-4c35)c2)c1 ; N#Cc1cccc(-c2cccc(-c3nc(-c4ccccc4)nc(-c4ccc5c6c(cccc46)-c4ccccc4-5)n3)c2)c1-c1cccc(-c2cccc(-c3ccc4c(c3)-c3cccc5cccc-4c35)c2)c1 ; N#Cc1cccc(-c2ccccc2-c2nc(-c3ccccc3)nc(-c3cccc(-c4cccc5ccccc45)c3)n2)c1-c1cccc(-c2ccccc2-c2cccc3ccc4ccccc4c23)c1 ; N#Cc1cccc(-c2cccc(-c3nc(-c4ccccc4)nc(-c4ccccc4-c4cccc5ccccc45)n3)c2)c1-c1ccc(-c2cccc(-c3cc4ccccc4c4ccccc34)c2)cc1]

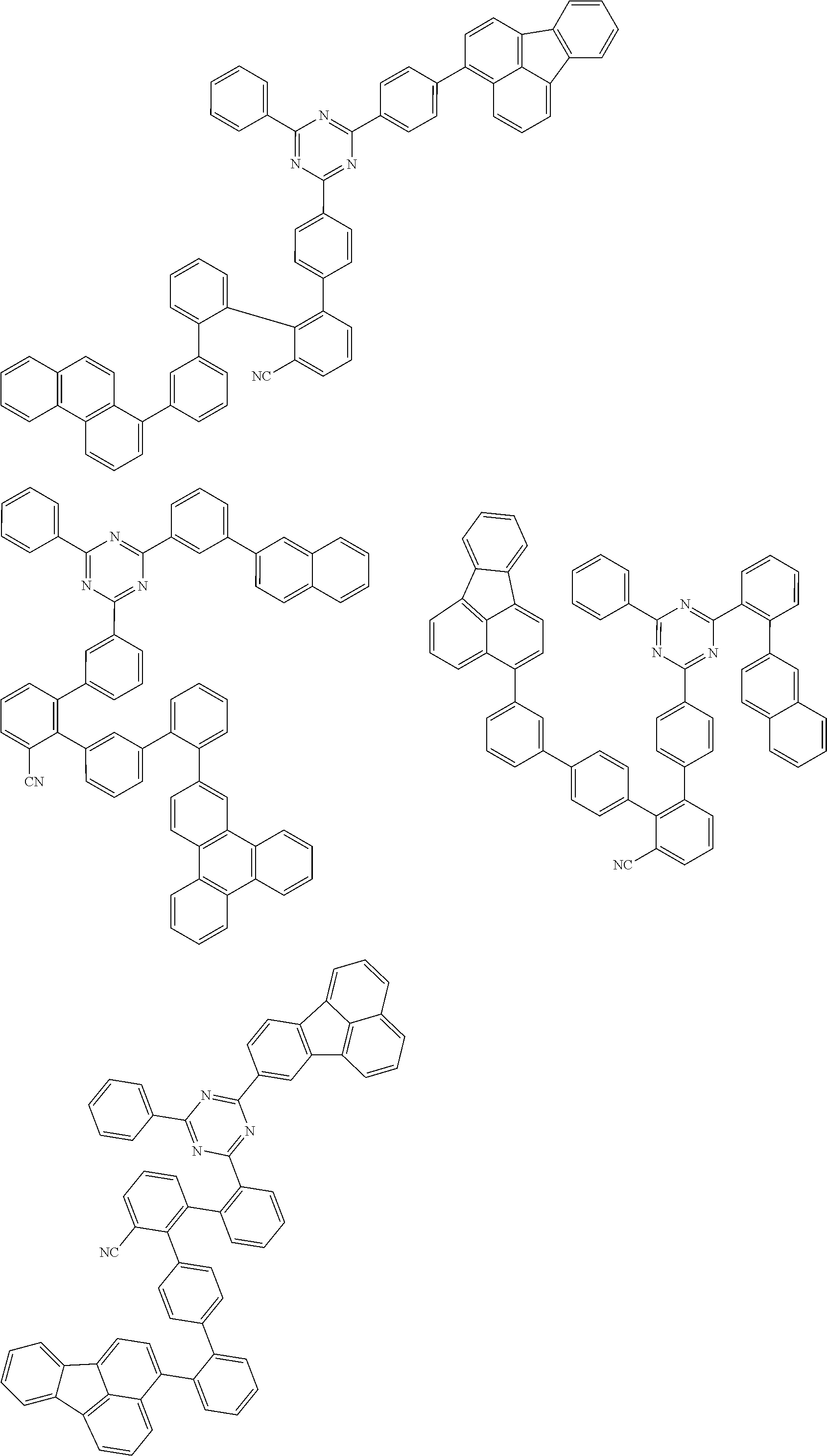 [SMILES: N#Cc1cccc(-c2ccc(-c3nc(-c4ccccc4)nc(-c4ccc(-c5ccc6c7c(cccc57)-c5ccccc5-6)cc4)n3)cc2)c1-c1ccccc1-c1cccc(-c2cccc3c2ccc2ccccc23)c1 ; N#Cc1cccc(-c2cccc(-c3nc(-c4ccccc4)nc(-c4cccc(-c5ccc6ccccc6c5)c4)n3)c2)c1-c1cccc(-c2ccccc2-c2ccc3c4ccccc4c4ccccc4c3c2)c1 ; N#Cc1cccc(-c2ccc(-c3nc(-c4ccccc4)nc(-c4ccccc4-c4ccc5ccccc5c4)n3)cc2)c1-c1ccc(-c2cccc(-c3ccc4c5c(cccc35)-c3ccccc3-4)c2)cc1 ; N#Cc1cccc(-c2ccccc2-c2nc(-c3ccccc3)nc(-c3ccc4c(c3)-c3cccc5cccc-4c35)n2)c1-c1ccc(-c2ccccc2-c2ccc3c4c(cccc24)-c2ccccc2-3)cc1]

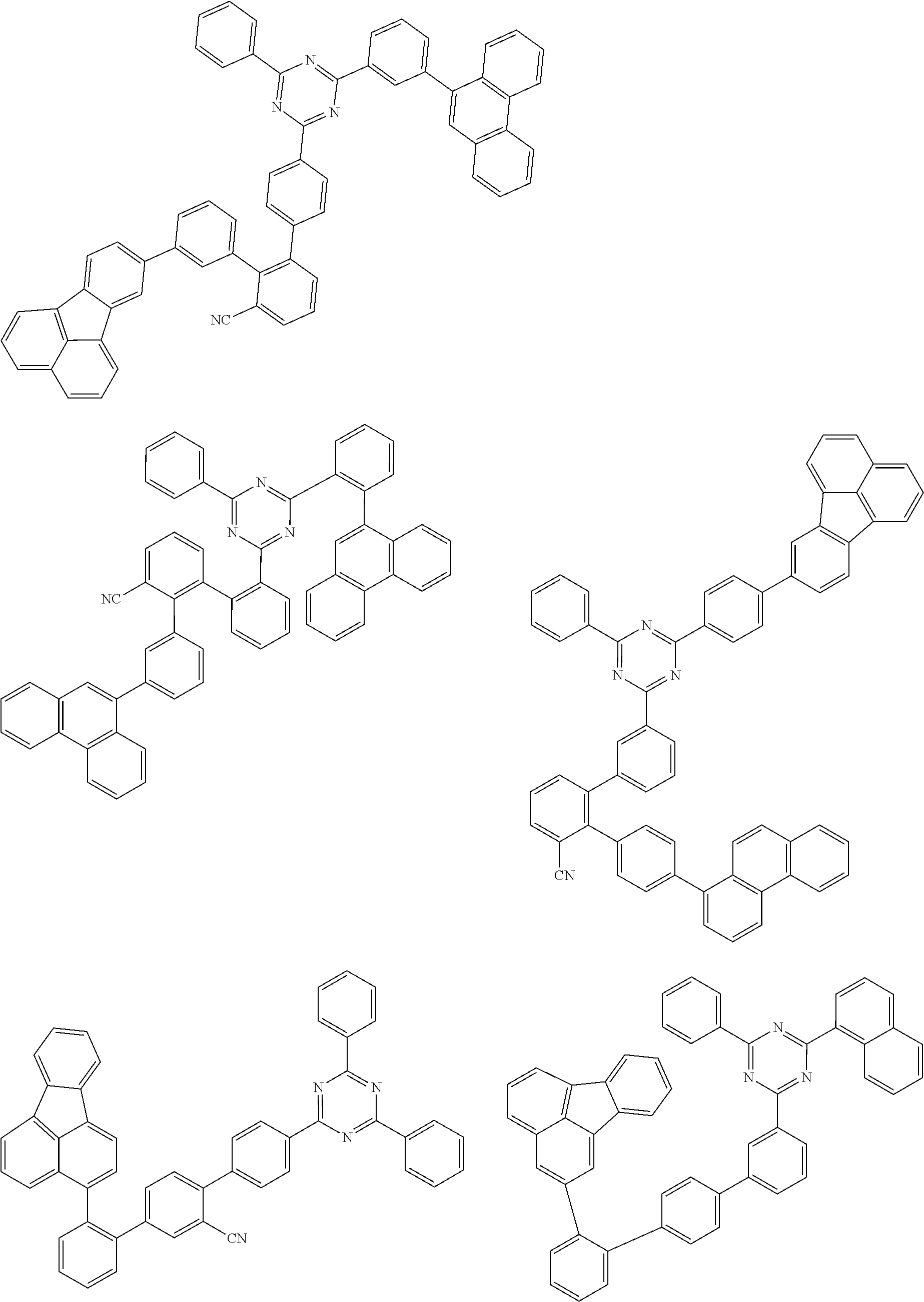

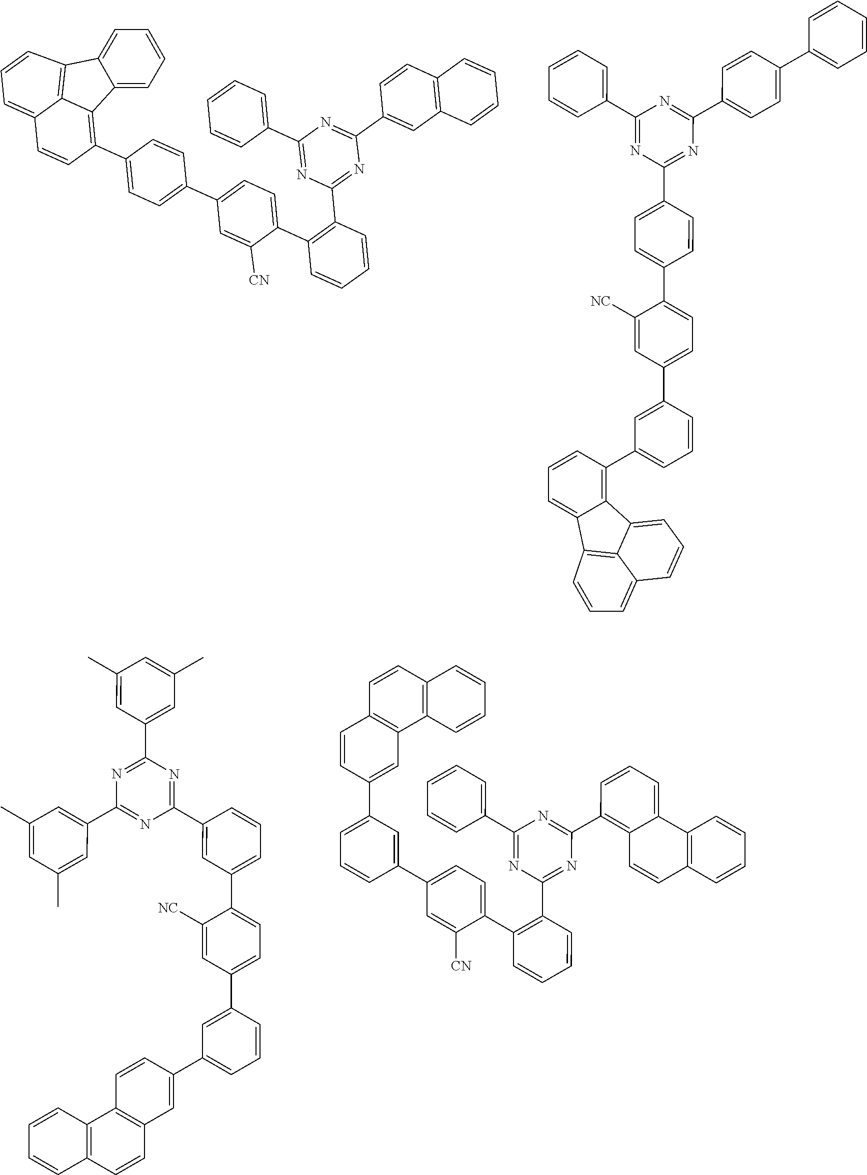 [SMILES: N#Cc1cc(-c2ccc(-c3ccc4cccc5c4c3-c3ccccc3-5)cc2)ccc1-c1ccccc1-c1nc(-c2ccccc2)nc(-c2ccc3ccccc3c2)n1 ; N#Cc1cc(-c2cccc(-c3cccc4c3-c3cccc5cccc-4c35)c2)ccc1-c1ccc(-c2nc(-c3ccccc3)nc(-c3ccc(-c4ccccc4)cc3)n2)cc1 ; Cc1cc(C)cc(-c2nc(-c3cc(C)cc(C)c3)nc(-c3cccc(-c4ccc(-c5cccc(-c6ccc7c(ccc8ccccc87)c6)c5)cc4C#N)c3)n2)c1 ; N#Cc1cc(-c2cccc(-c3ccc4ccc5ccccc5c4c3)c2)ccc1-c1ccccc1-c1nc(-c2ccccc2)nc(-c2cccc3c2ccc2ccccc23)n1]

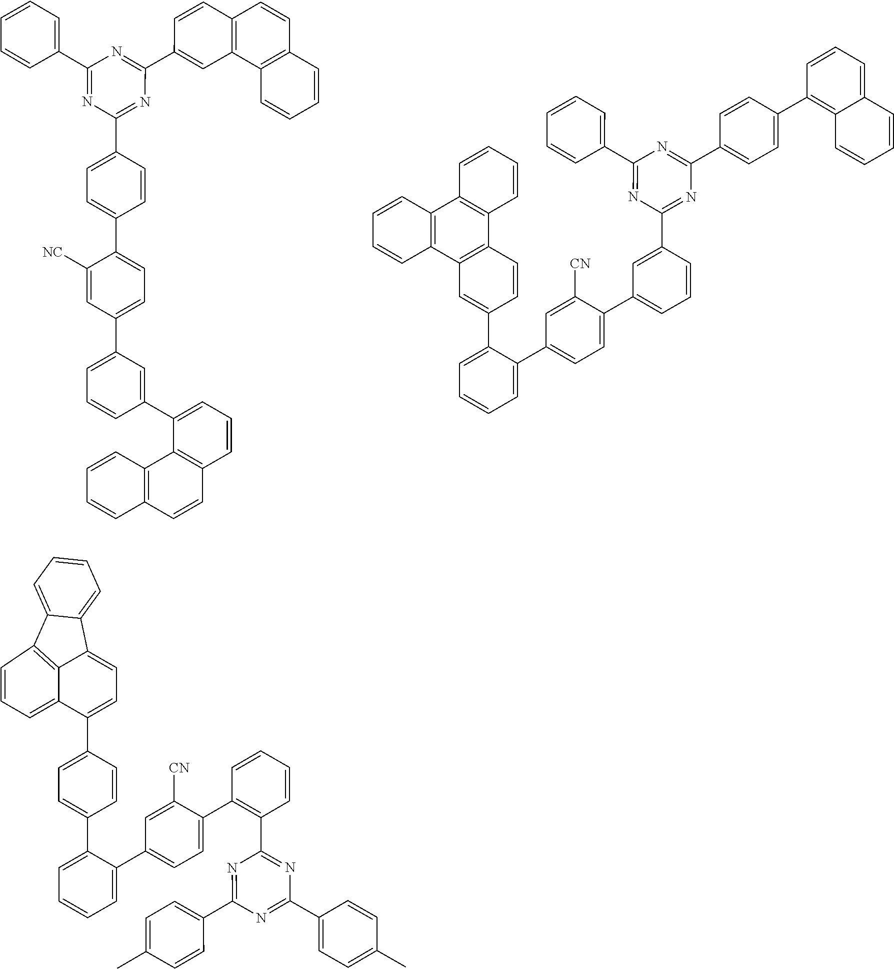 [SMILES: N#Cc1cc(-c2cccc(-c3cccc4ccc5ccccc5c34)c2)ccc1-c1ccc(-c2nc(-c3ccccc3)nc(-c3ccc4ccc5ccccc5c4c3)n2)cc1 ; N#Cc1cc(-c2ccccc2-c2ccc3c4ccccc4c4ccccc4c3c2)ccc1-c1cccc(-c2nc(-c3ccccc3)nc(-c3ccc(-c4cccc5ccccc45)cc3)n2)c1 ; Cc1ccc(-c2nc(-c3ccc(C)cc3)nc(-c3ccccc3-c3ccc(-c4ccccc4-c4ccc(-c5ccc6c7c(cccc57)-c5ccccc5-6)cc4)cc3C#N)n2)cc1]

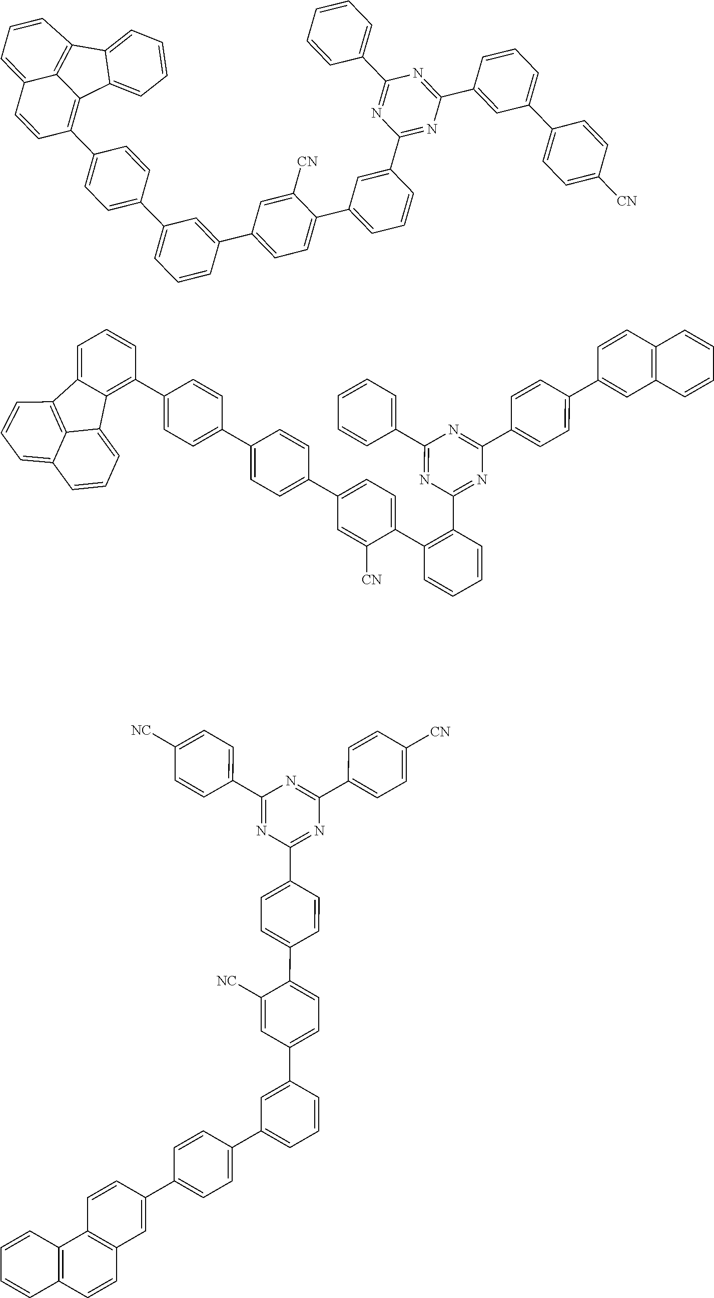 [SMILES: N#Cc1ccc(-c2cccc(-c3nc(-c4ccccc4)nc(-c4cccc(-c5ccc(-c6cccc(-c7ccc(-c8ccc9cccc%10c9c8-c8ccccc8-%10)cc7)c6)cc5C#N)c4)n3)c2)cc1 ; N#Cc1cc(-c2ccc(-c3ccc(-c4cccc5c4-c4cccc6cccc-5c46)cc3)cc2)ccc1-c1ccccc1-c1nc(-c2ccccc2)nc(-c2ccc(-c3ccc4ccccc4c3)cc2)n1 ; N#Cc1ccc(-c2nc(-c3ccc(C#N)cc3)nc(-c3ccc(-c4ccc(-c5cccc(-c6ccc(-c7ccc8c(ccc9ccccc98)c7)cc6)c5)cc4C#N)cc3)n2)cc1]

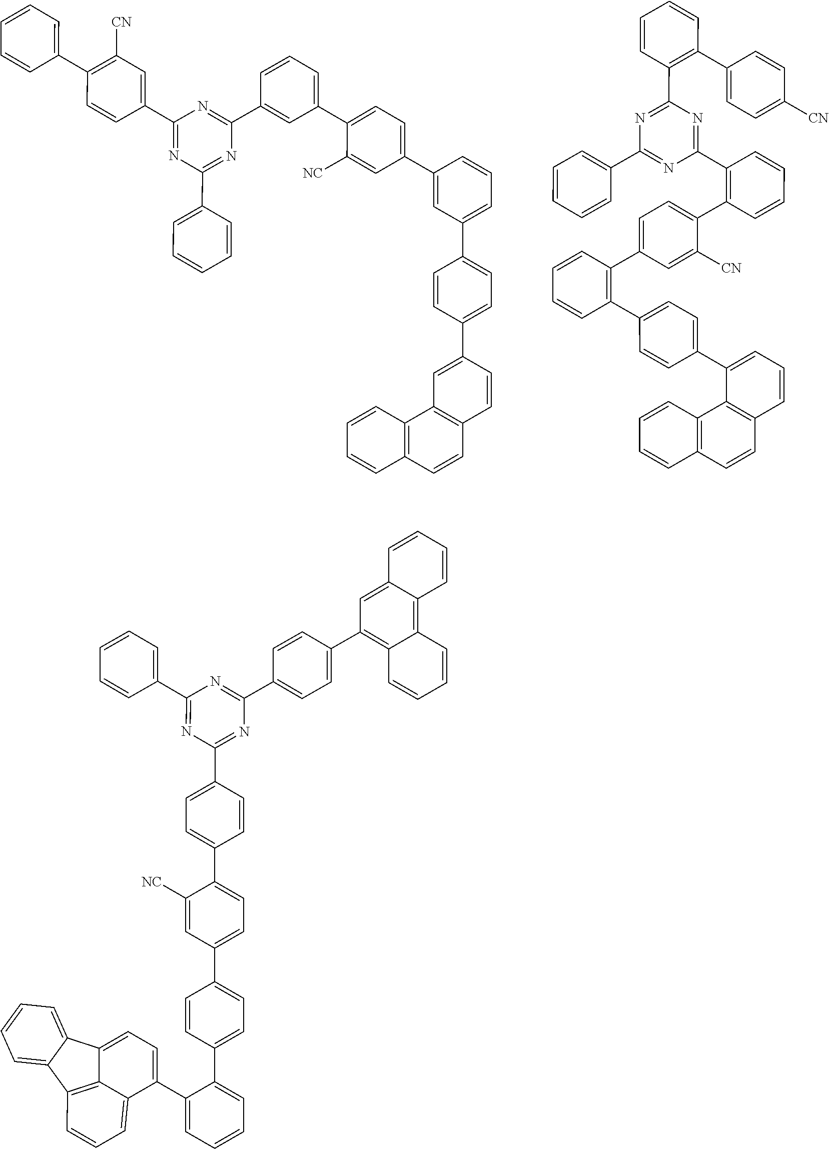 [SMILES: N#Cc1cc(-c2cccc(-c3ccc(-c4ccc5ccc6ccccc6c5c4)cc3)c2)ccc1-c1cccc(-c2nc(-c3ccccc3)nc(-c3ccc(-c4ccccc4)c(C#N)c3)n2)c1 ; N#Cc1ccc(-c2ccccc2-c2nc(-c3ccccc3)nc(-c3ccccc3-c3ccc(-c4ccccc4-c4ccc(-c5cccc6ccc7ccccc7c56)cc4)cc3C#N)n2)cc1 ; N#Cc1cc(-c2ccc(-c3ccccc3-c3ccc4c5c(cccc35)-c3ccccc3-4)cc2)ccc1-c1ccc(-c2nc(-c3ccccc3)nc(-c3ccc(-c4cc5ccccc5c5ccccc45)cc3)n2)cc1]

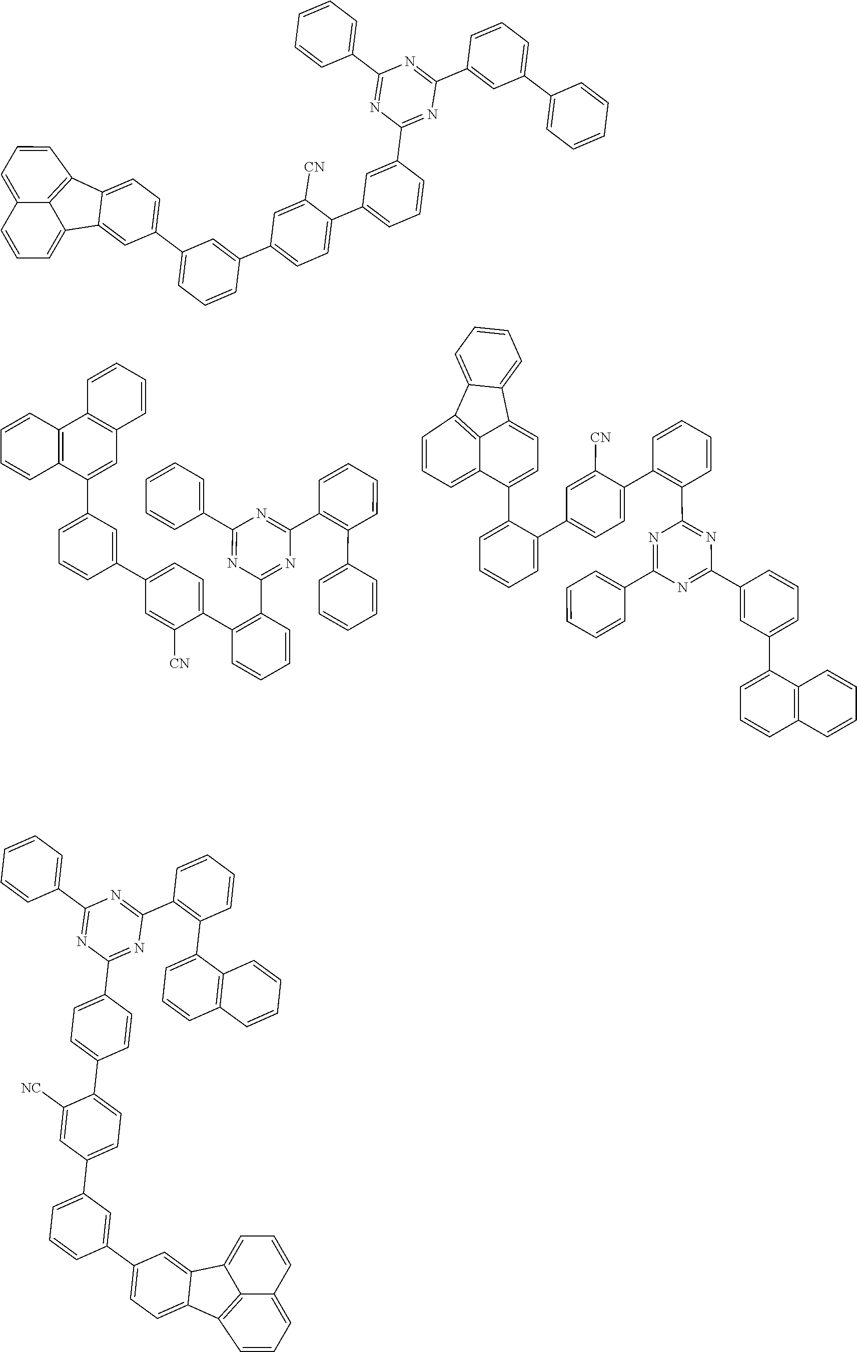

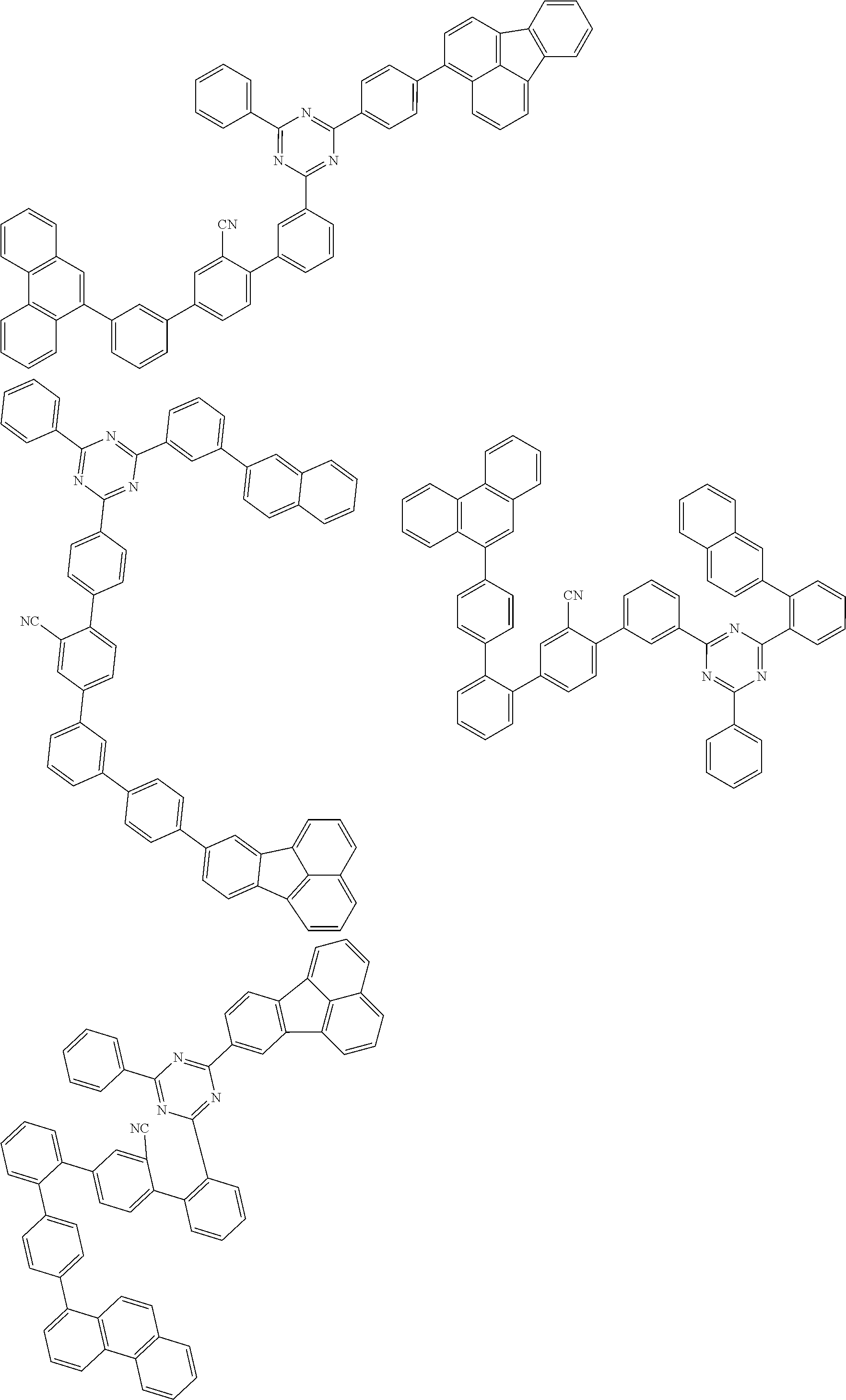

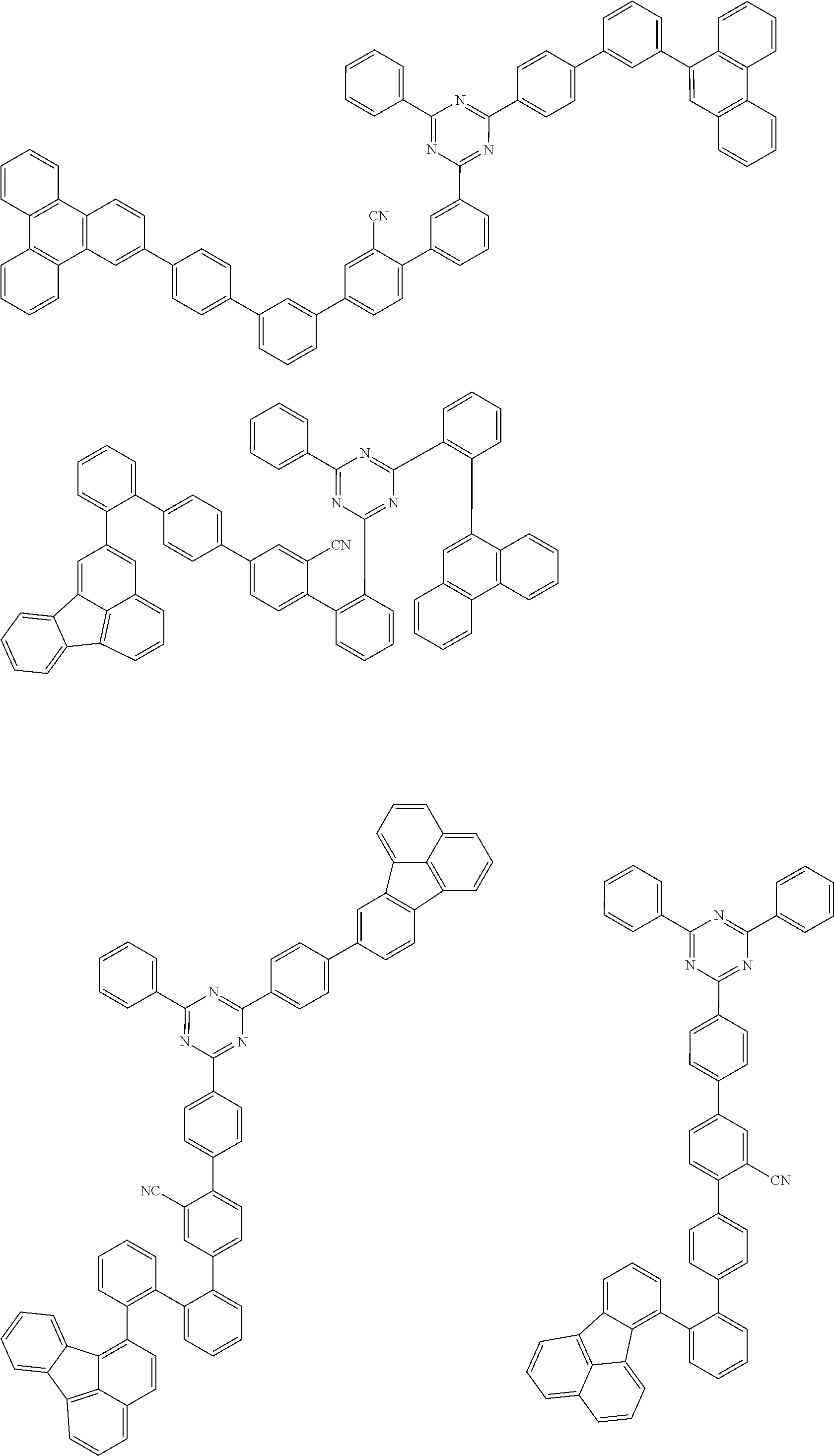

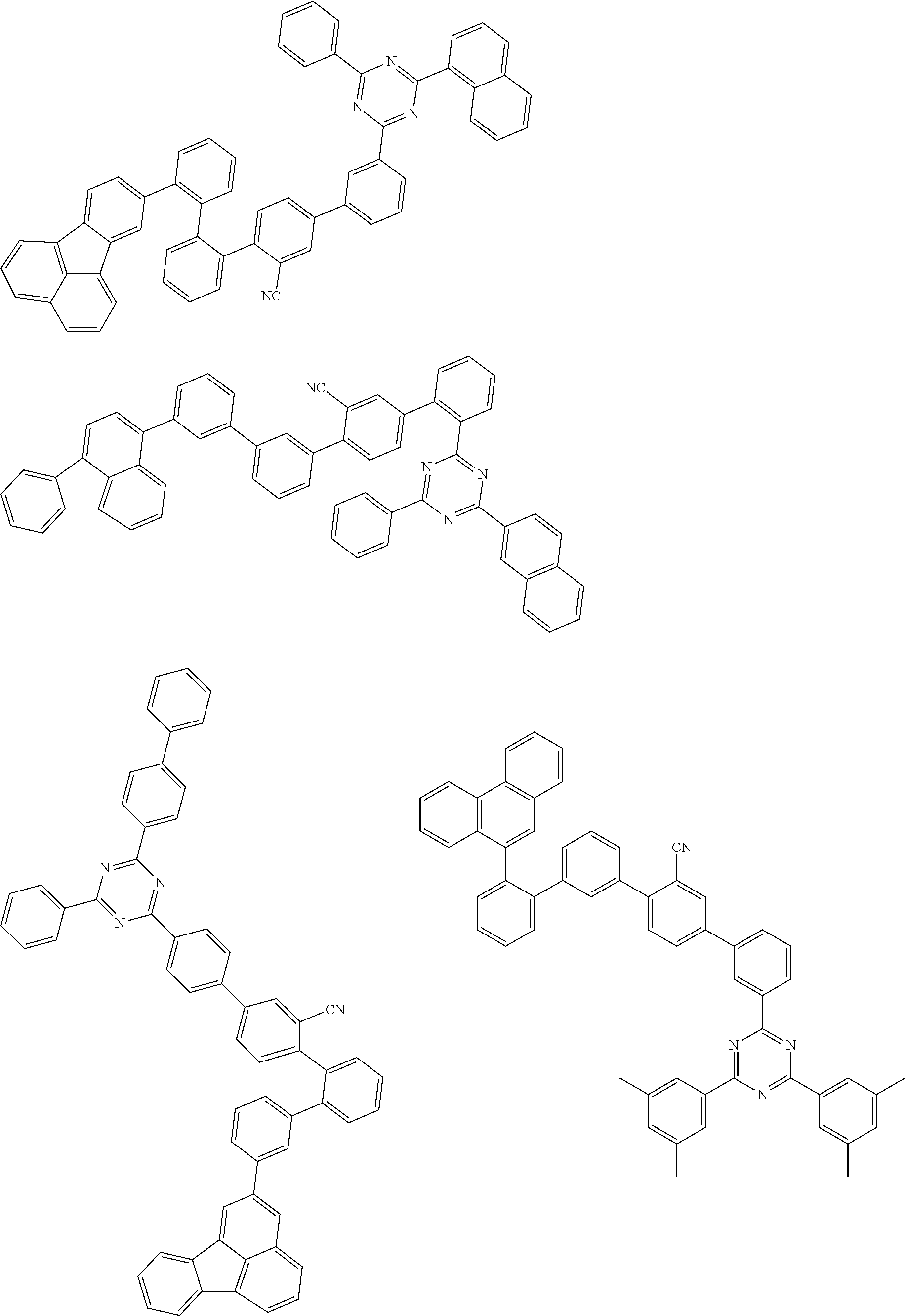 [SMILES: N#Cc1cc(-c2cccc(-c3nc(-c4ccccc4)nc(-c4cccc5ccccc45)n3)c2)ccc1-c1ccccc1-c1ccccc1-c1ccc2c(c1)-c1cccc3cccc-2c13 ; N#Cc1cc(-c2ccccc2-c2nc(-c3ccccc3)nc(-c3ccc4ccccc4c3)n2)ccc1-c1cccc(-c2cccc(-c3ccc4c5c(cccc35)-c3ccccc3-4)c2)c1 ; N#Cc1cc(-c2ccc(-c3nc(-c4ccccc4)nc(-c4ccc(-c5ccccc5)cc4)n3)cc2)ccc1-c1ccccc1-c1cccc(-c2cc3c4c(cccc4c2)-c2ccccc2-3)c1 ; Cc1cc(C)cc(-c2nc(-c3cc(C)cc(C)c3)nc(-c3cccc(-c4ccc(-c5cccc(-c6ccccc6-c6cc7ccccc7c7ccccc67)c5)c(C#N)c4)c3)n2)c1]

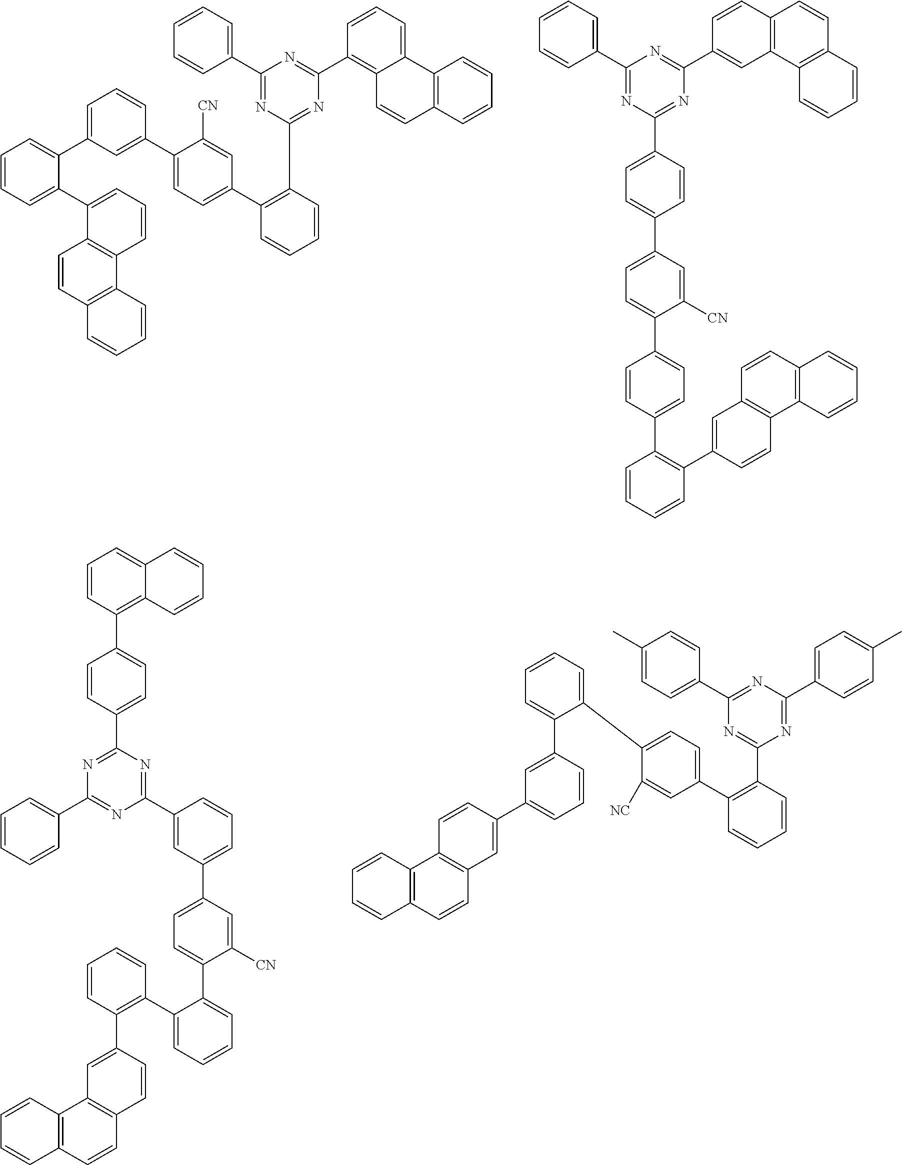 [SMILES: N#Cc1cc(-c2ccccc2-c2nc(-c3ccccc3)nc(-c3cccc4c3ccc3ccccc34)n2)ccc1-c1cccc(-c2ccccc2-c2cccc3c2ccc2ccccc23)c1 ; N#Cc1cc(-c2ccc(-c3nc(-c4ccccc4)nc(-c4ccc5ccc6ccccc6c5c4)n3)cc2)ccc1-c1ccc(-c2ccccc2-c2ccc3c(ccc4ccccc43)c2)cc1 ; N#Cc1cc(-c2cccc(-c3nc(-c4ccccc4)nc(-c4ccc(-c5cccc6ccccc56)cc4)n3)c2)ccc1-c1ccccc1-c1ccccc1-c1ccc2ccc3ccccc3c2c1 ; Cc1ccc(-c2nc(-c3ccc(C)cc3)nc(-c3ccccc3-c3ccc(-c4ccccc4-c4cccc(-c5ccc6c(ccc7ccccc76)c5)c4)c(C#N)c3)n2)cc1]

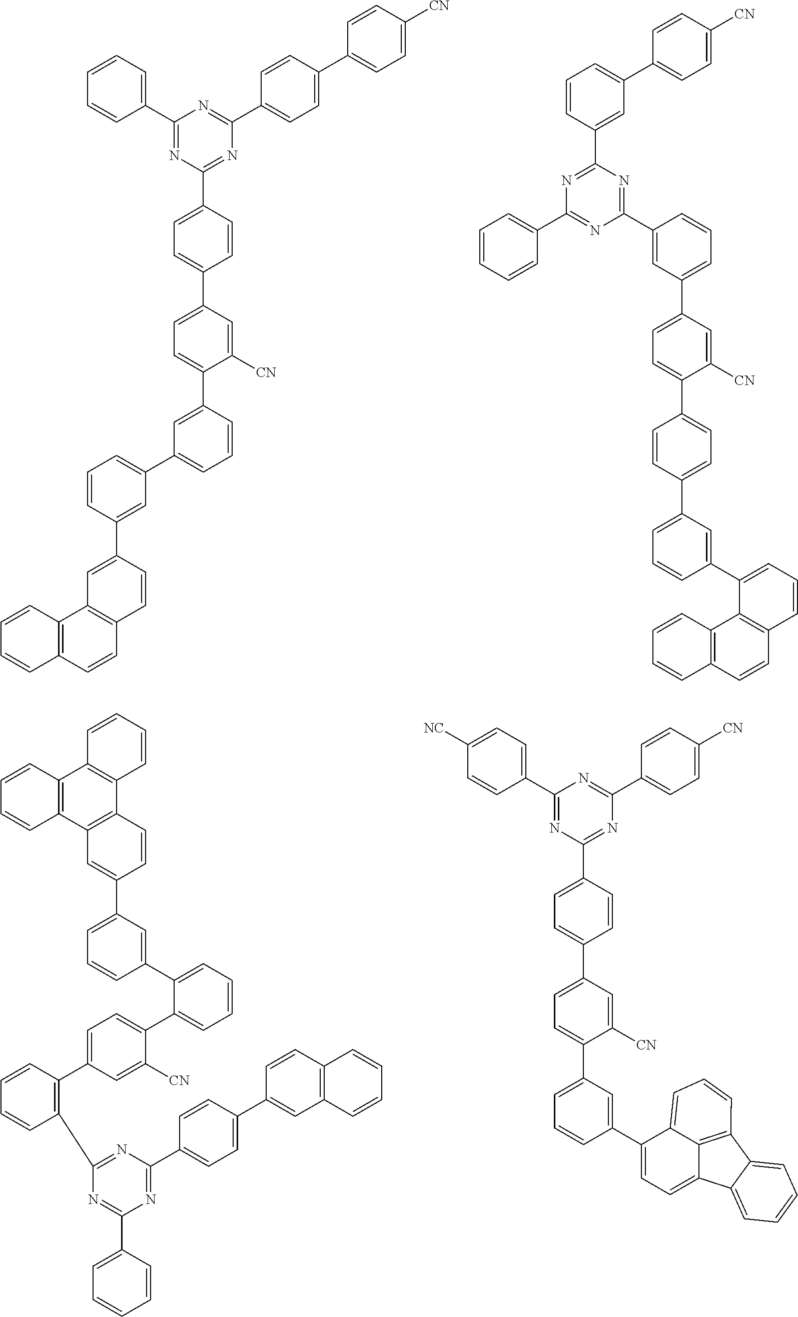 [SMILES: N#Cc1ccc(-c2ccc(-c3nc(-c4ccccc4)nc(-c4ccc(-c5ccc(-c6cccc(-c7cccc(-c8ccc9ccc%10ccccc%10c9c8)c7)c6)c(C#N)c5)cc4)n3)cc2)cc1 ; N#Cc1ccc(-c2cccc(-c3nc(-c4ccccc4)nc(-c4cccc(-c5ccc(-c6ccc(-c7cccc(-c8cccc9ccc%10ccccc%10c89)c7)cc6)c(C#N)c5)c4)n3)c2)cc1 ; N#Cc1cc(-c2ccccc2-c2nc(-c3ccccc3)nc(-c3ccc(-c4ccc5ccccc5c4)cc3)n2)ccc1-c1ccccc1-c1cccc(-c2ccc3c4ccccc4c4ccccc4c3c2)c1 ; N#Cc1ccc(-c2nc(-c3ccc(C#N)cc3)nc(-c3ccc(-c4ccc(-c5cccc(-c6ccc7c8c(cccc68)-c6ccccc6-7)c5)c(C#N)c4)cc3)n2)cc1]

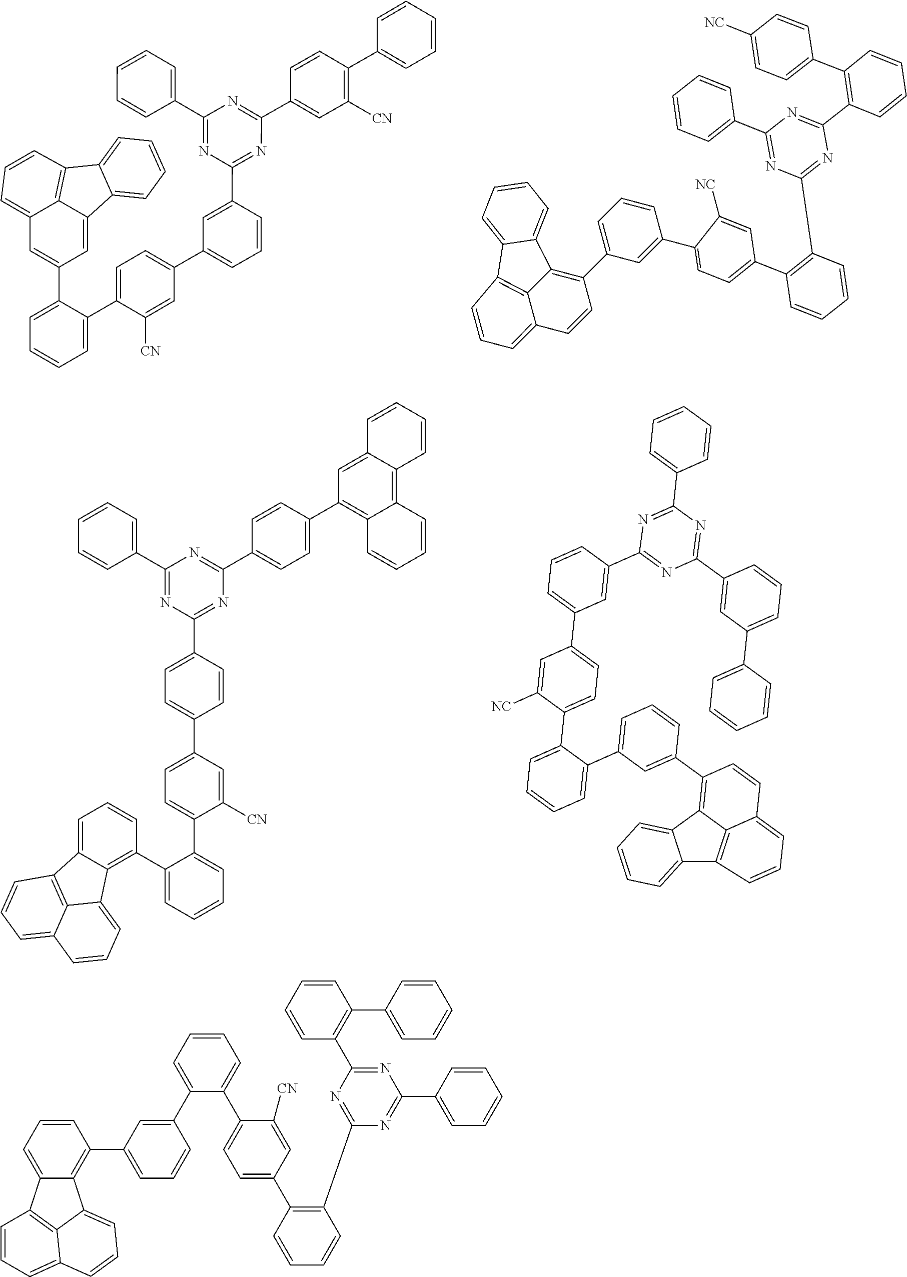

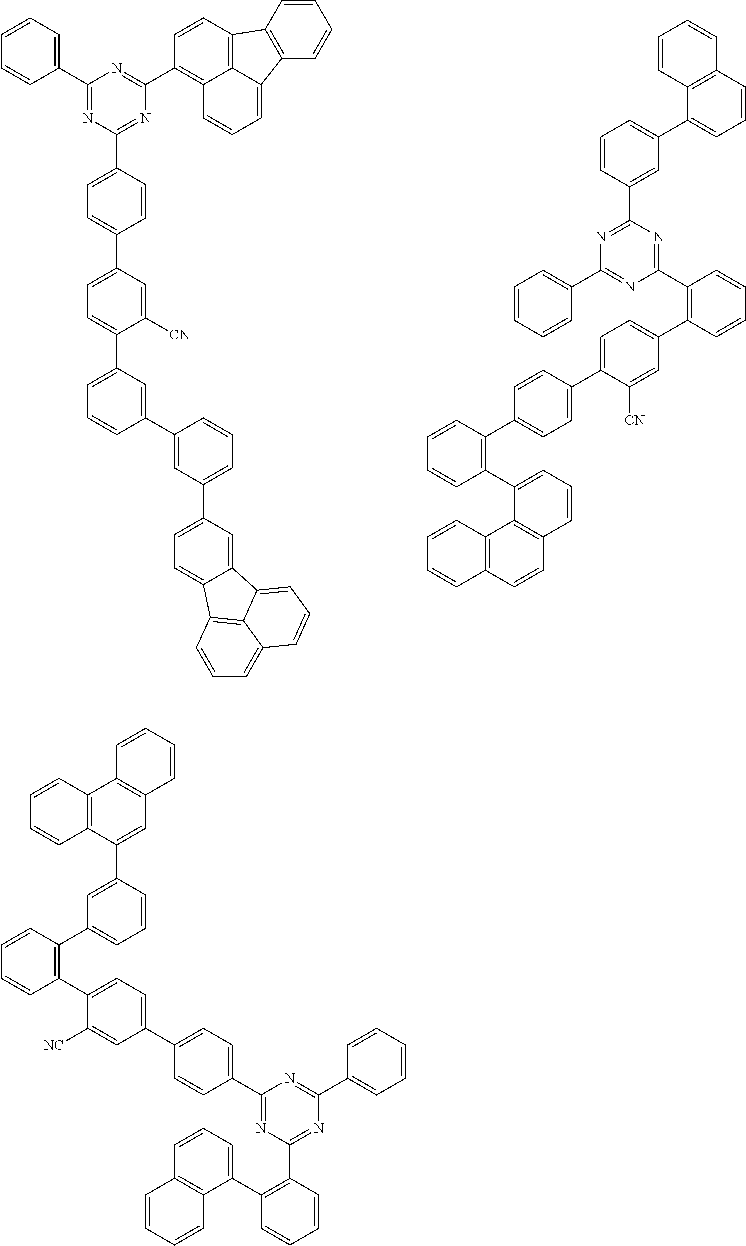 [SMILES: N#Cc1cc(-c2ccc(-c3nc(-c4ccccc4)nc(-c4ccc5c6c(cccc46)-c4ccccc4-5)n3)cc2)ccc1-c1cccc(-c2cccc(-c3ccc4c(c3)-c3cccc5cccc-4c35)c2)c1 ; N#Cc1cc(-c2ccccc2-c2nc(-c3ccccc3)nc(-c3cccc(-c4cccc5ccccc45)c3)n2)ccc1-c1ccc(-c2ccccc2-c2cccc3ccc4ccccc4c23)cc1 ; N#Cc1cc(-c2ccc(-c3nc(-c4ccccc4)nc(-c4ccccc4-c4cccc5ccccc45)n3)cc2)ccc1-c1ccccc1-c1cccc(-c2cc3ccccc3c3ccccc23)c1]

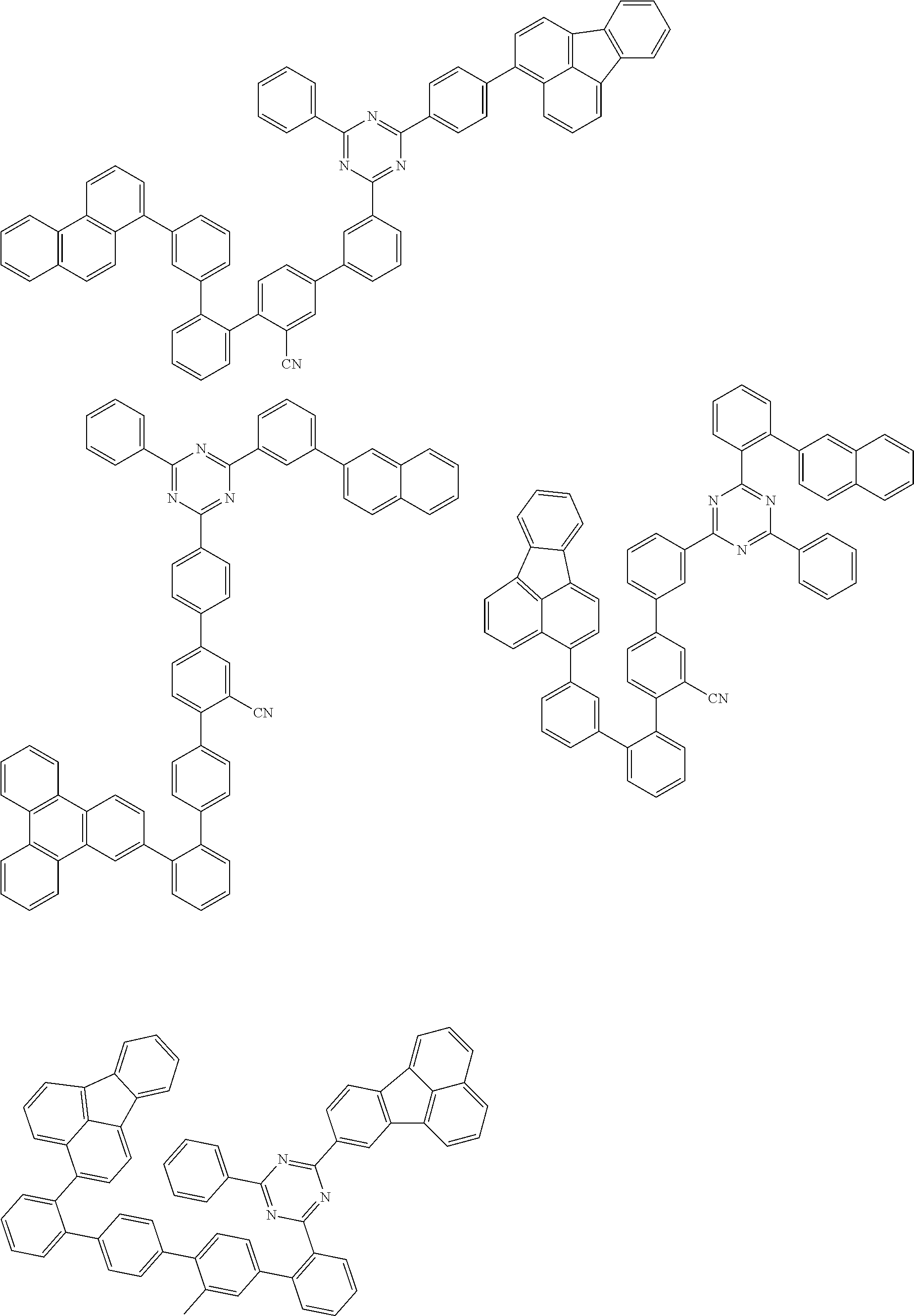

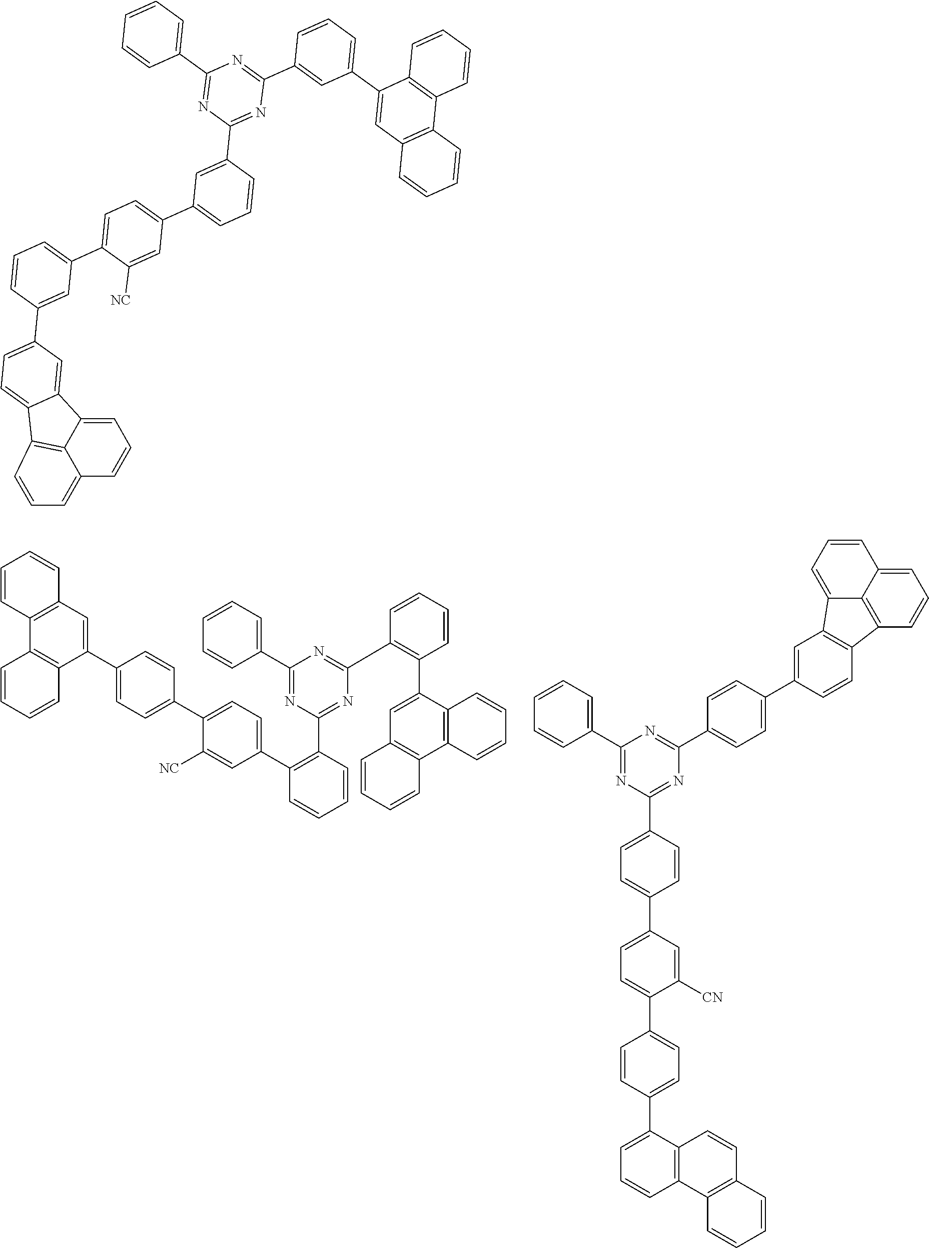

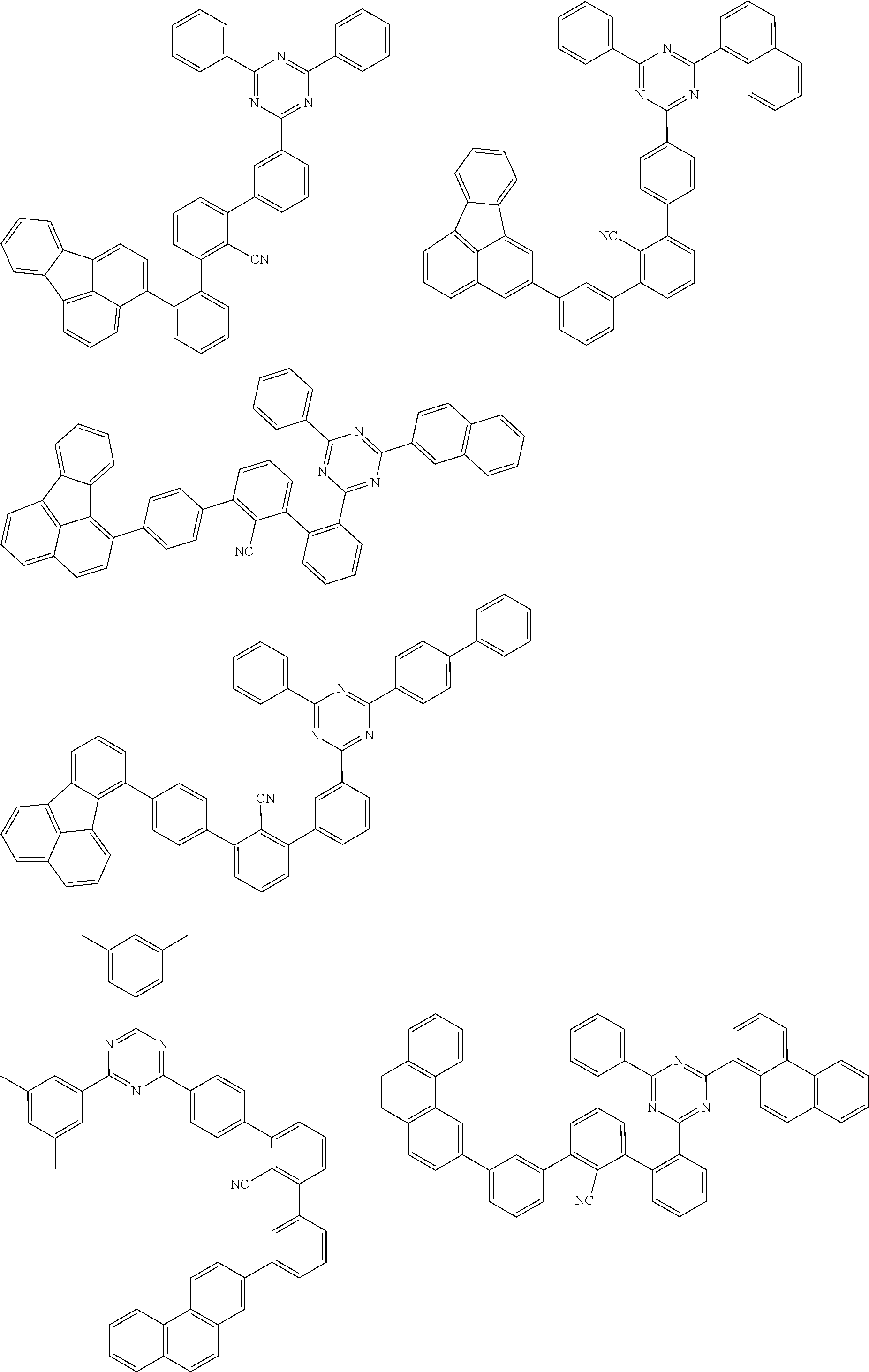

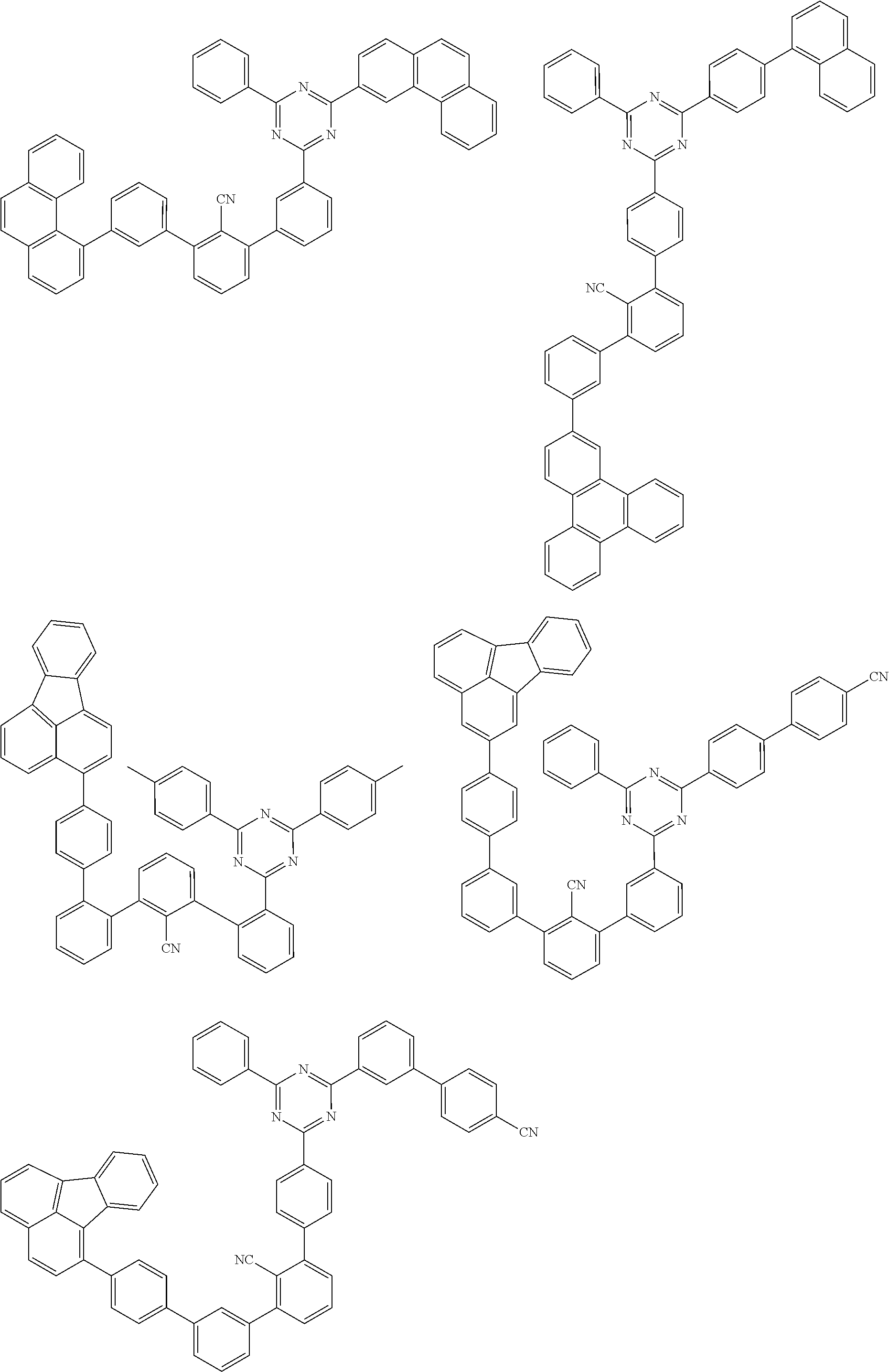

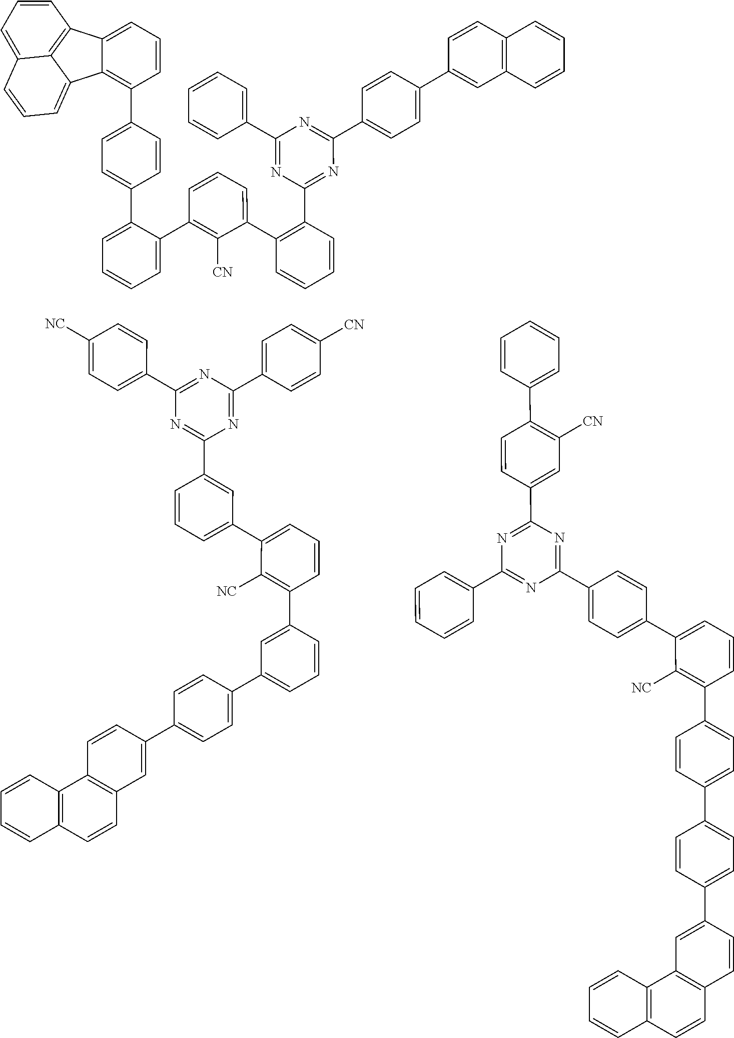

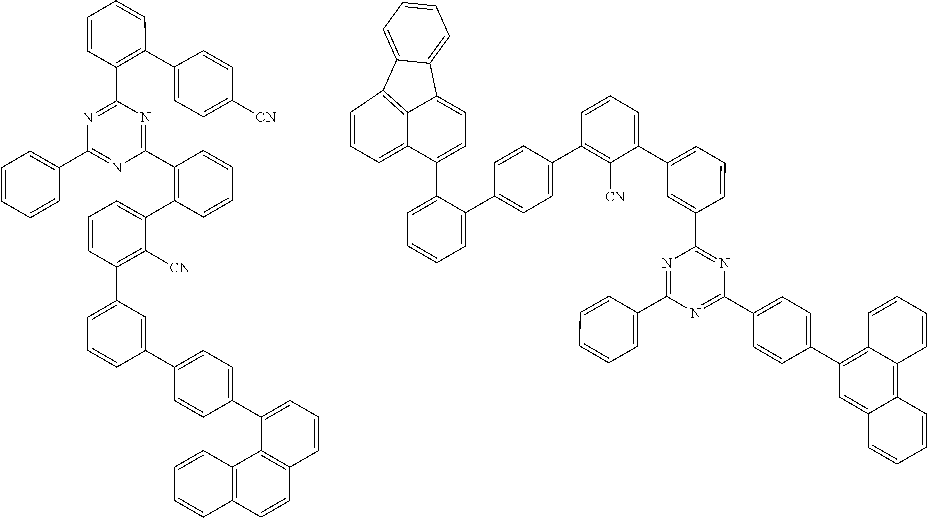 [SMILES: N#Cc1ccc(-c2ccccc2-c2nc(-c3ccccc3)nc(-c3ccccc3-c3cccc(-c4cccc(-c5ccc(-c6cccc7ccc8ccccc8c67)cc5)c4)c3C#N)n2)cc1 ; N#Cc1c(-c2ccc(-c3ccccc3-c3ccc4c5c(cccc35)-c3ccccc3-4)cc2)cccc1-c1cccc(-c2nc(-c3ccccc3)nc(-c3ccc(-c4cc5ccccc5c5ccccc45)cc3)n2)c1]

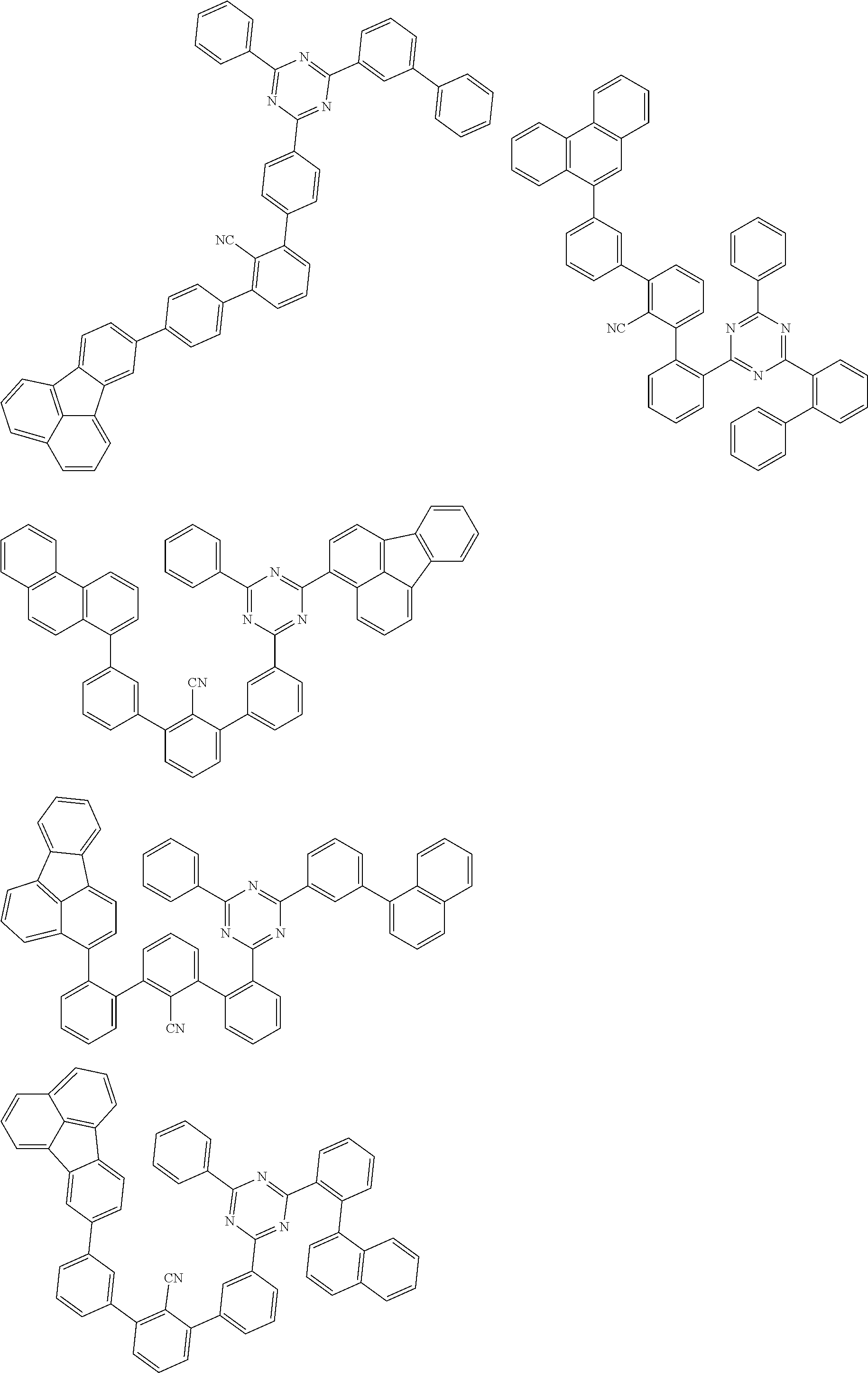

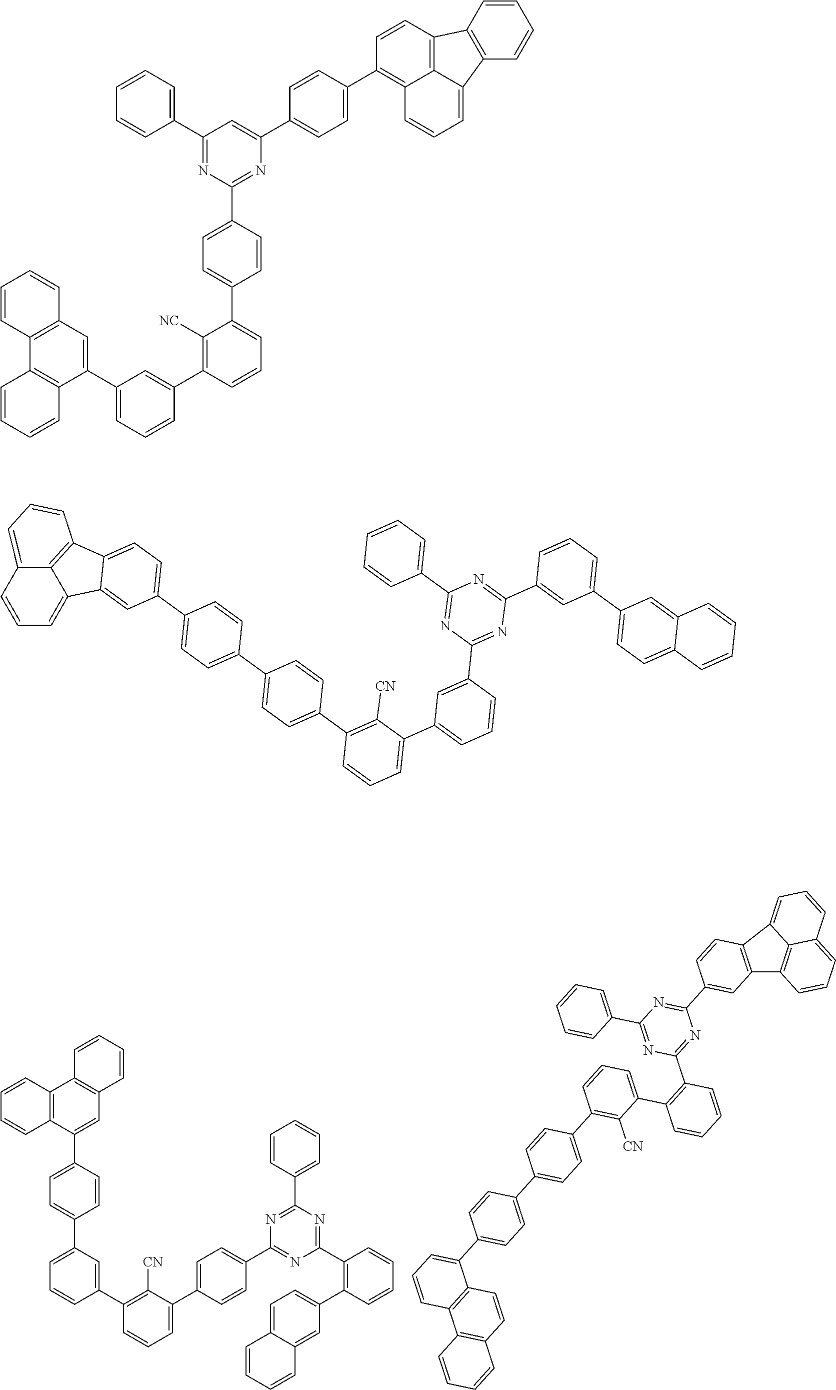

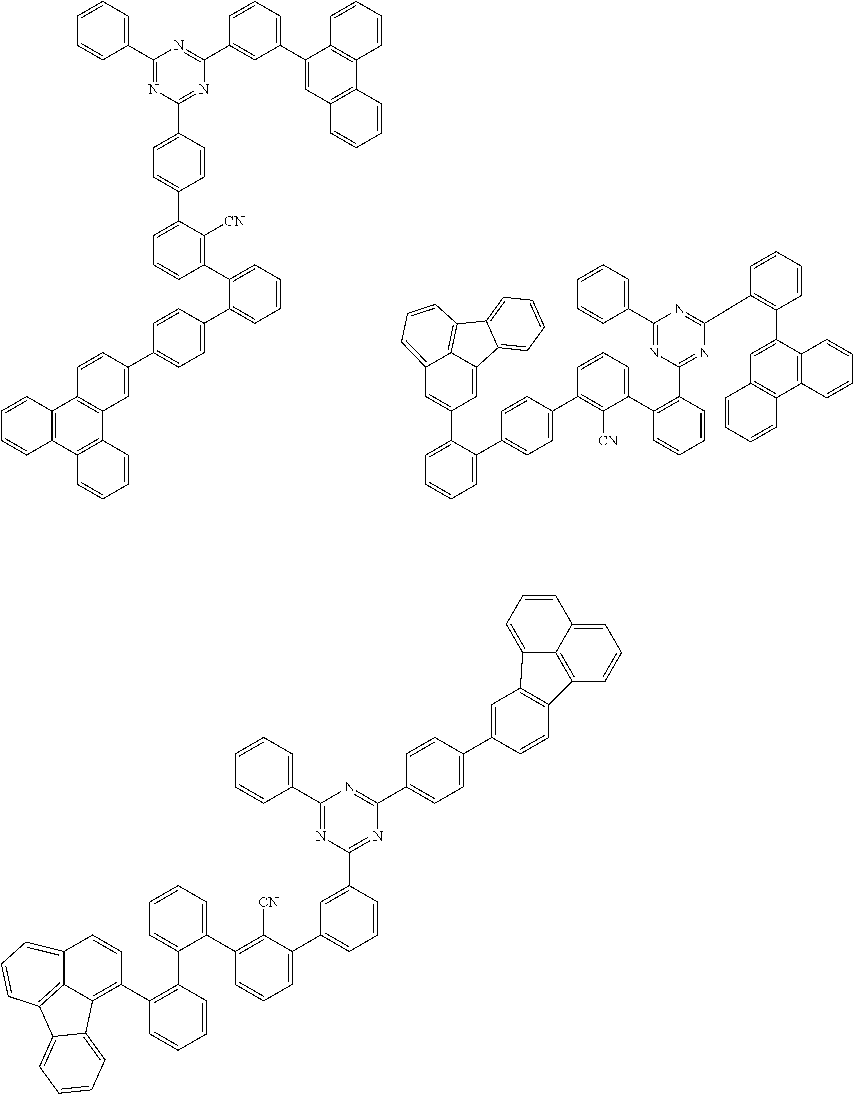 [SMILES: N#Cc1c(-c2ccc(-c3nc(-c4ccccc4)nc(-c4cccc(-c5cc6ccccc6c6ccccc56)c4)n3)cc2)cccc1-c1ccccc1-c1ccc(-c2ccc3c4ccccc4c4ccccc4c3c2)cc1 ; N#Cc1c(-c2ccc(-c3ccccc3-c3cc4c5c(cccc5c3)-c3ccccc3-4)cc2)cccc1-c1ccccc1-c1nc(-c2ccccc2)nc(-c2ccccc2-c2cc3ccccc3c3ccccc23)n1 ; N#Cc1c(-c2cccc(-c3nc(-c4ccccc4)nc(-c4ccc(-c5ccc6c(c5)-c5cccc7cccc-6c57)cc4)n3)c2)cccc1-c1ccccc1-c1ccccc1-c1ccc2cccc3c2c1-c1ccccc1-3]

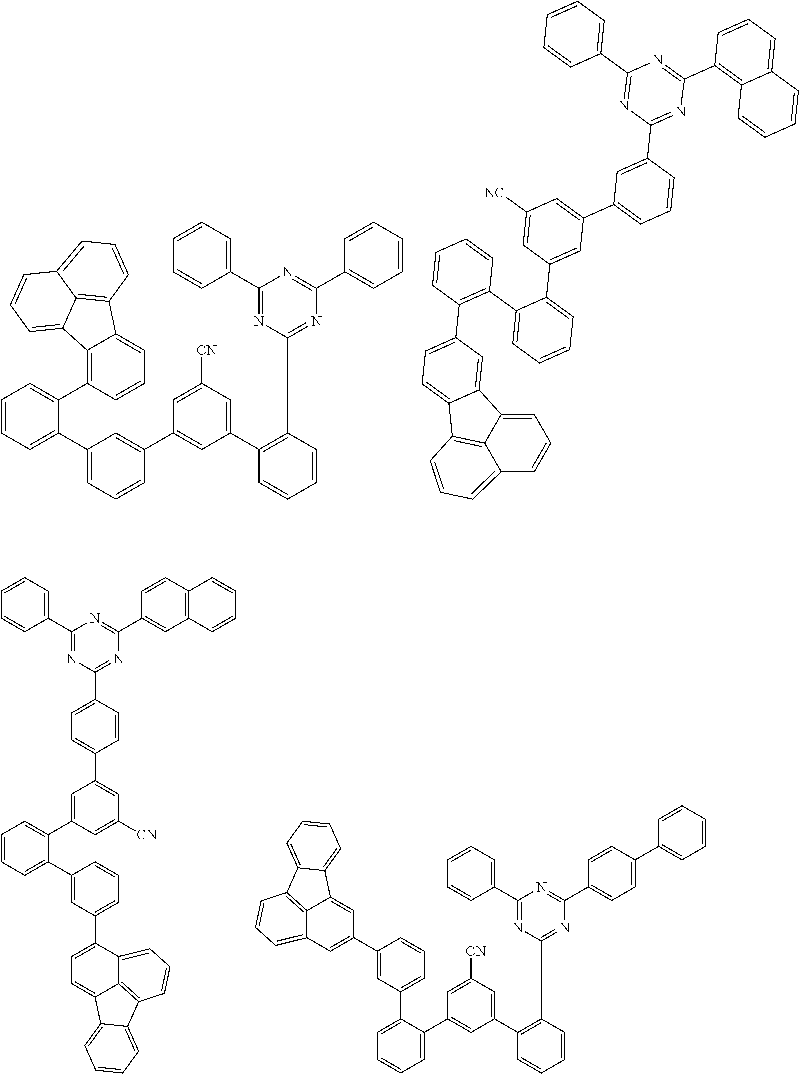

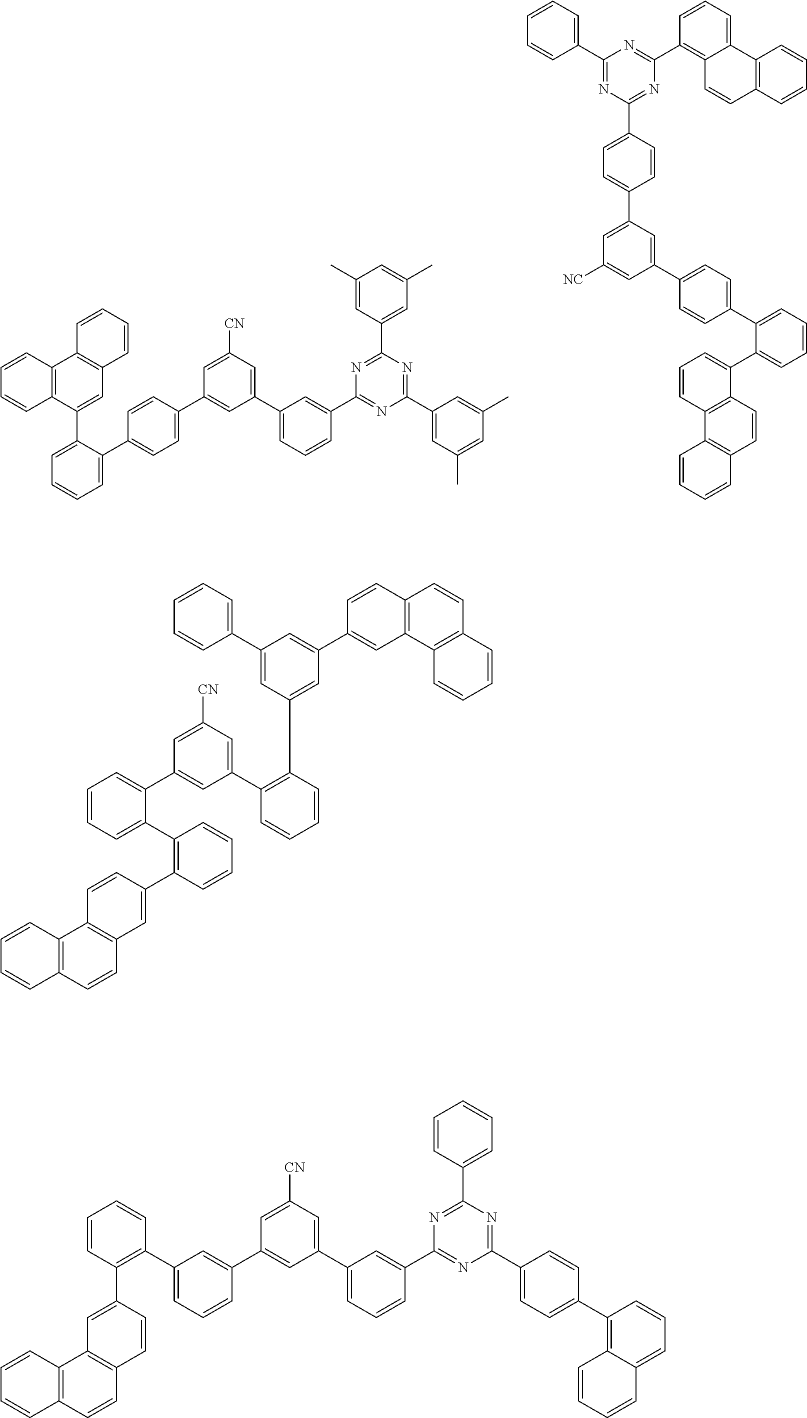 [SMILES: Cc1cc(C)cc(-c2nc(-c3cc(C)cc(C)c3)nc(-c3cccc(-c4cc(C#N)cc(-c5ccc(-c6ccccc6-c6cc7ccccc7c7ccccc67)cc5)c4)c3)n2)c1 ; N#Cc1cc(-c2ccc(-c3nc(-c4ccccc4)nc(-c4cccc5c4ccc4ccccc45)n3)cc2)cc(-c2ccc(-c3ccccc3-c3cccc4c3ccc3ccccc34)cc2)c1 ; N#Cc1cc(-c2ccccc2-c2cc(-c3ccccc3)cc(-c3ccc4ccc5ccccc5c4c3)c2)cc(-c2ccccc2-c2ccccc2-c2ccc3c(ccc4ccccc43)c2)c1 ; N#Cc1cc(-c2cccc(-c3nc(-c4ccccc4)nc(-c4ccc(-c5cccc6ccccc56)cc4)n3)c2)cc(-c2cccc(-c3ccccc3-c3ccc4ccc5ccccc5c4c3)c2)c1]

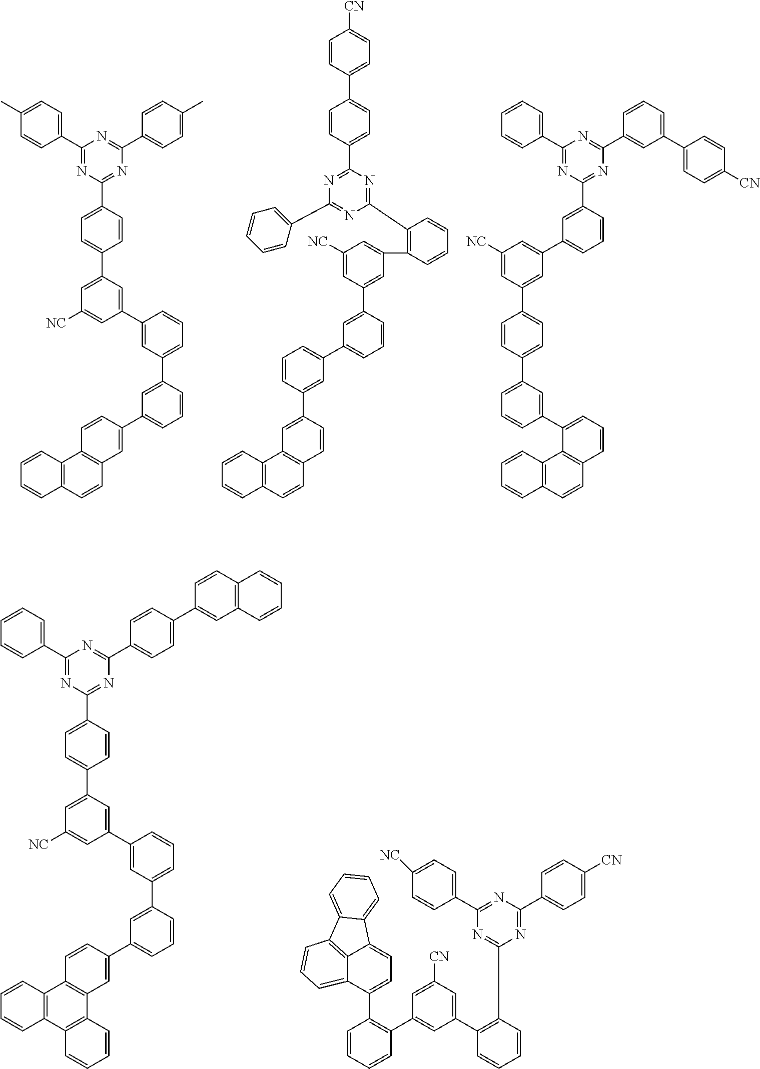

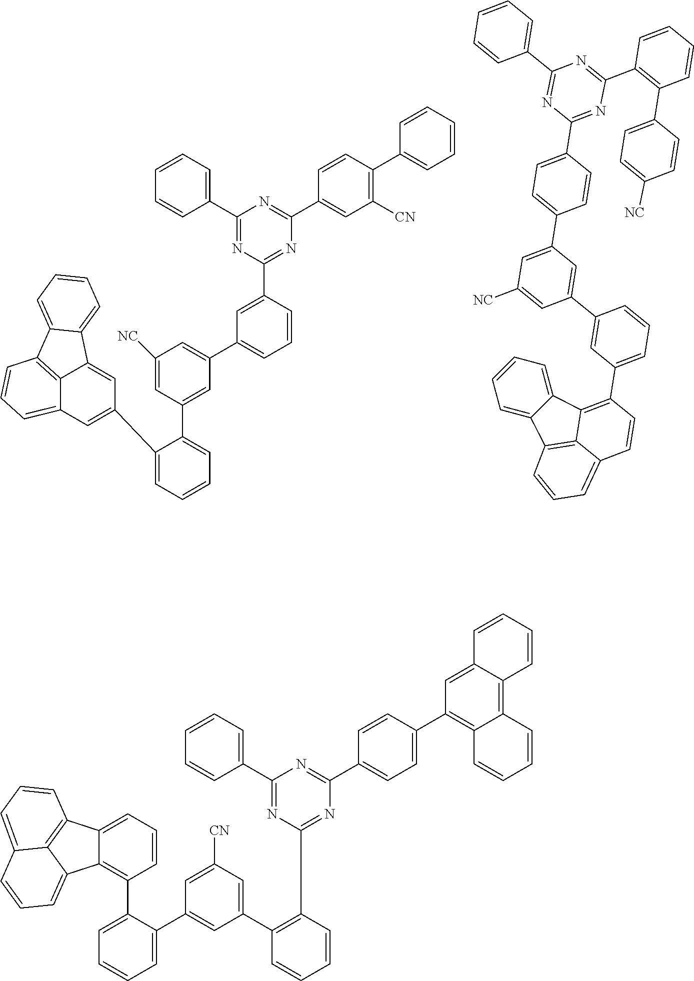 [SMILES: N#Cc1cc(-c2cccc(-c3nc(-c4ccccc4)nc(-c4ccc(-c5ccccc5)c(C#N)c4)n3)c2)cc(-c2ccccc2-c2cc3c4c(cccc4c2)-c2ccccc2-3)c1 ; N#Cc1ccc(-c2ccccc2-c2nc(-c3ccccc3)nc(-c3ccc(-c4cc(C#N)cc(-c5cccc(-c6ccc7cccc8c7c6-c6ccccc6-8)c5)c4)cc3)n2)cc1 ; N#Cc1cc(-c2ccccc2-c2nc(-c3ccccc3)nc(-c3ccc(-c4cc5ccccc5c5ccccc45)cc3)n2)cc(-c2ccccc2-c2cccc3c2-c2cccc4cccc-3c24)c1]

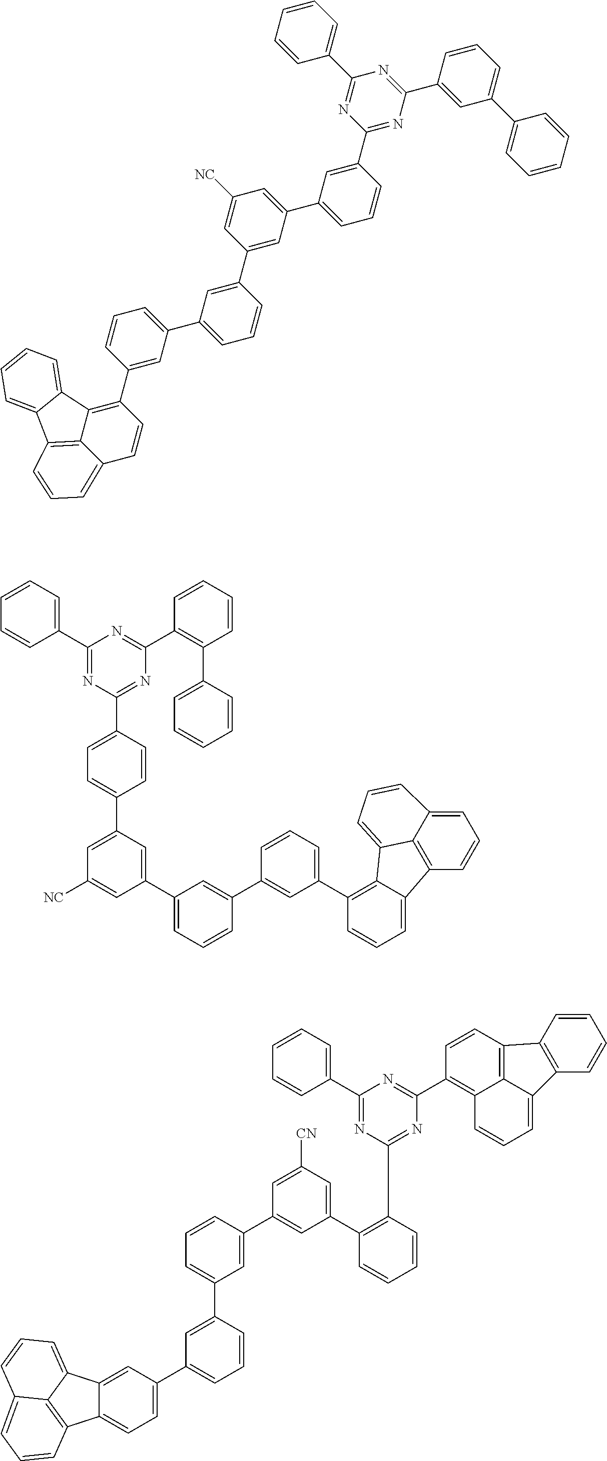 [SMILES: N#Cc1cc(-c2cccc(-c3cccc(-c4ccc5cccc6c5c4-c4ccccc4-6)c3)c2)cc(-c2cccc(-c3nc(-c4ccccc4)nc(-c4cccc(-c5ccccc5)c4)n3)c2)c1 ; N#Cc1cc(-c2ccc(-c3nc(-c4ccccc4)nc(-c4ccccc4-c4ccccc4)n3)cc2)cc(-c2cccc(-c3cccc(-c4cccc5c4-c4cccc6cccc-5c46)c3)c2)c1 ; N#Cc1cc(-c2cccc(-c3cccc(-c4ccc5c(c4)-c4cccc6cccc-5c46)c3)c2)cc(-c2ccccc2-c2nc(-c3ccccc3)nc(-c3ccc4c5c(cccc35)-c3ccccc3-4)n2)c1]

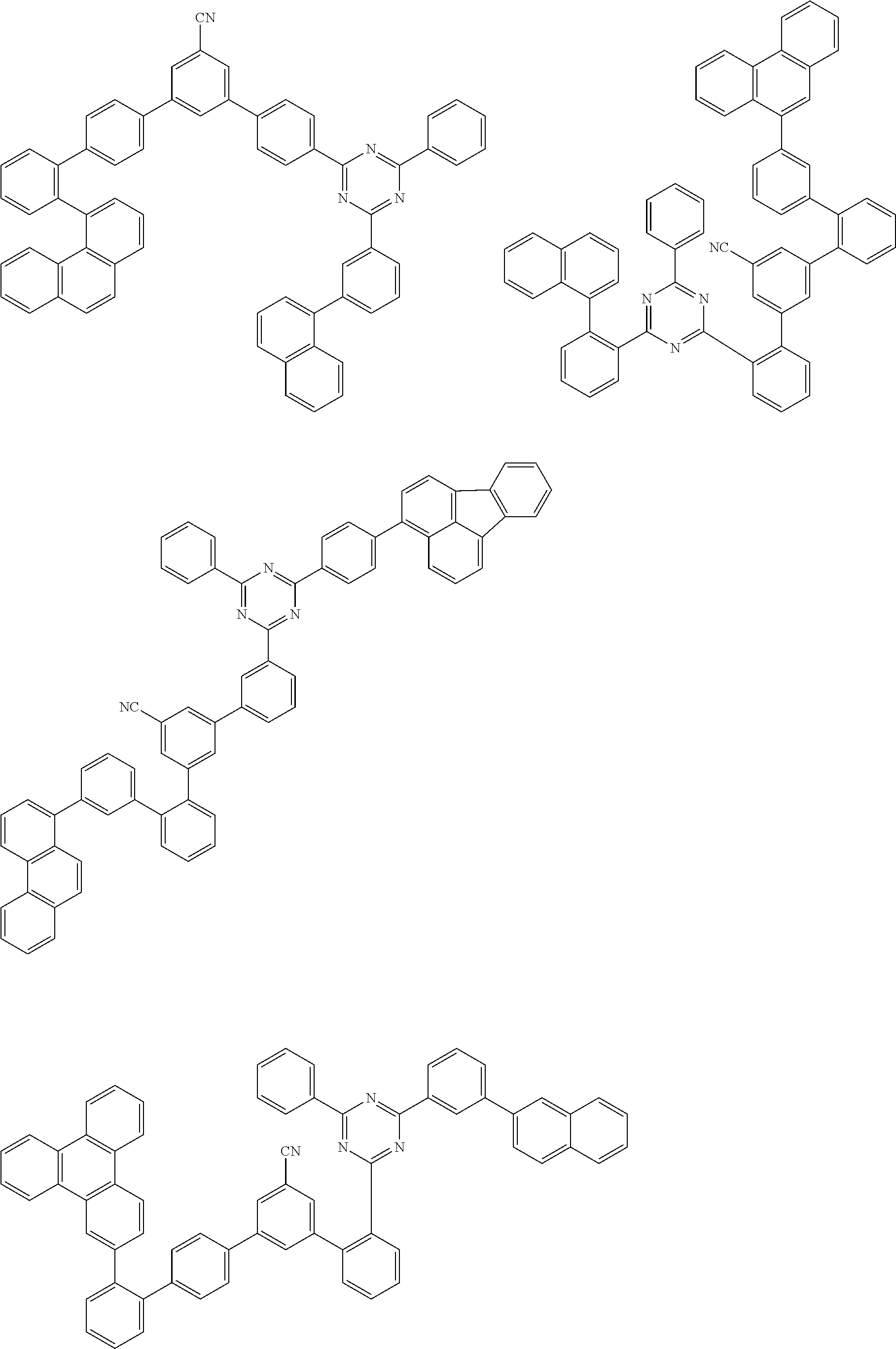 [SMILES: N#Cc1cc(-c2ccc(-c3nc(-c4ccccc4)nc(-c4cccc(-c5cccc6ccccc56)c4)n3)cc2)cc(-c2ccc(-c3ccccc3-c3cccc4ccc5ccccc5c34)cc2)c1 ; N#Cc1cc(-c2ccccc2-c2cccc(-c3cc4ccccc4c4ccccc34)c2)cc(-c2ccccc2-c2nc(-c3ccccc3)nc(-c3ccccc3-c3cccc4ccccc34)n2)c1 ; N#Cc1cc(-c2cccc(-c3nc(-c4ccccc4)nc(-c4ccc(-c5ccc6c7c(cccc57)-c5ccccc5-6)cc4)n3)c2)cc(-c2ccccc2-c2cccc(-c3cccc4c3ccc3ccccc34)c2)c1 ; N#Cc1cc(-c2ccc(-c3ccccc3-c3ccc4c5ccccc5c5ccccc5c4c3)cc2)cc(-c2ccccc2-c2nc(-c3ccccc3)nc(-c3cccc(-c4ccc5ccccc5c4)c3)n2)c1]

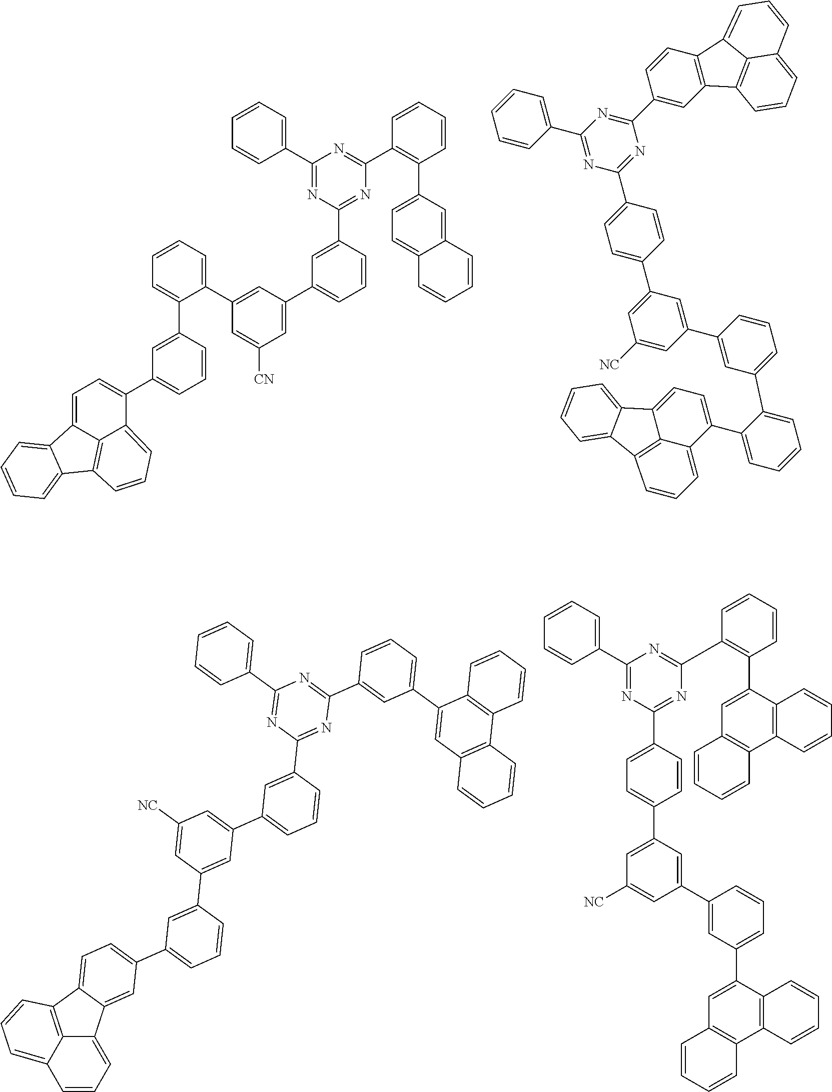

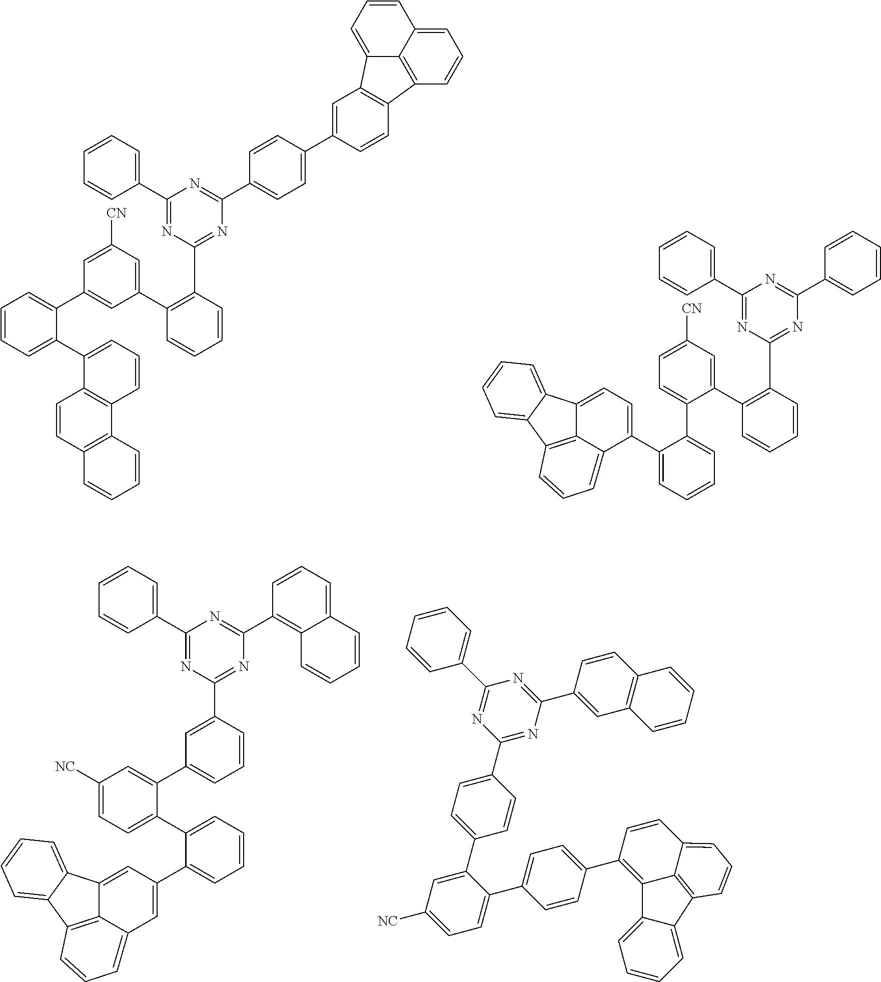 [SMILES: N#Cc1cc(-c2ccccc2-c2nc(-c3ccccc3)nc(-c3ccc(-c4ccc5c(c4)-c4cccc6cccc-5c46)cc3)n2)cc(-c2ccccc2-c2cccc3c2ccc2ccccc23)c1 ; N#Cc1ccc(-c2ccccc2-c2ccc3c4c(cccc24)-c2ccccc2-3)c(-c2ccccc2-c2nc(-c3ccccc3)nc(-c3ccccc3)n2)c1 ; N#Cc1ccc(-c2ccccc2-c2cc3c4c(cccc4c2)-c2ccccc2-3)c(-c2cccc(-c3nc(-c4ccccc4)nc(-c4cccc5ccccc45)n3)c2)c1 ; N#Cc1ccc(-c2ccc(-c3ccc4cccc5c4c3-c3ccccc3-5)cc2)c(-c2ccc(-c3nc(-c4ccccc4)nc(-c4ccc5ccccc5c4)n3)cc2)c1]

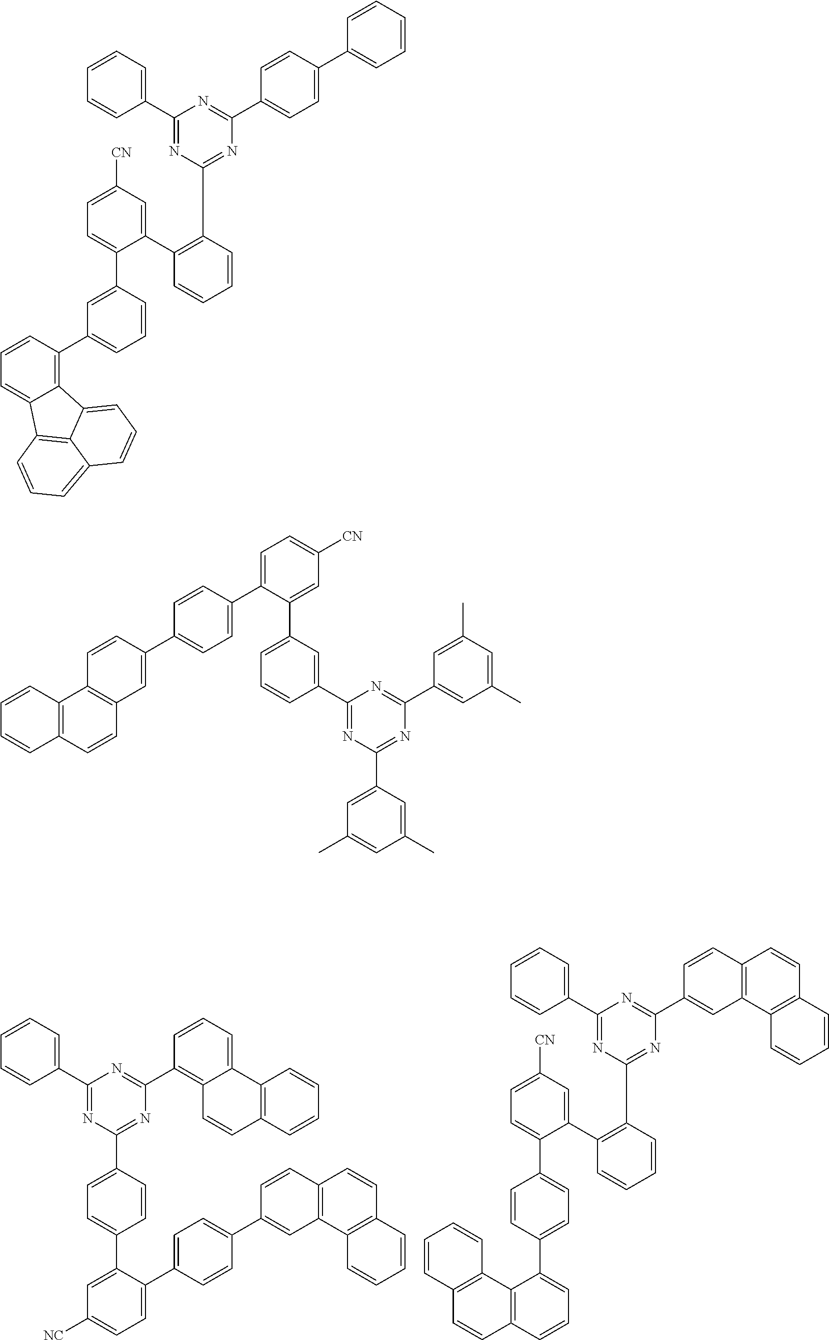

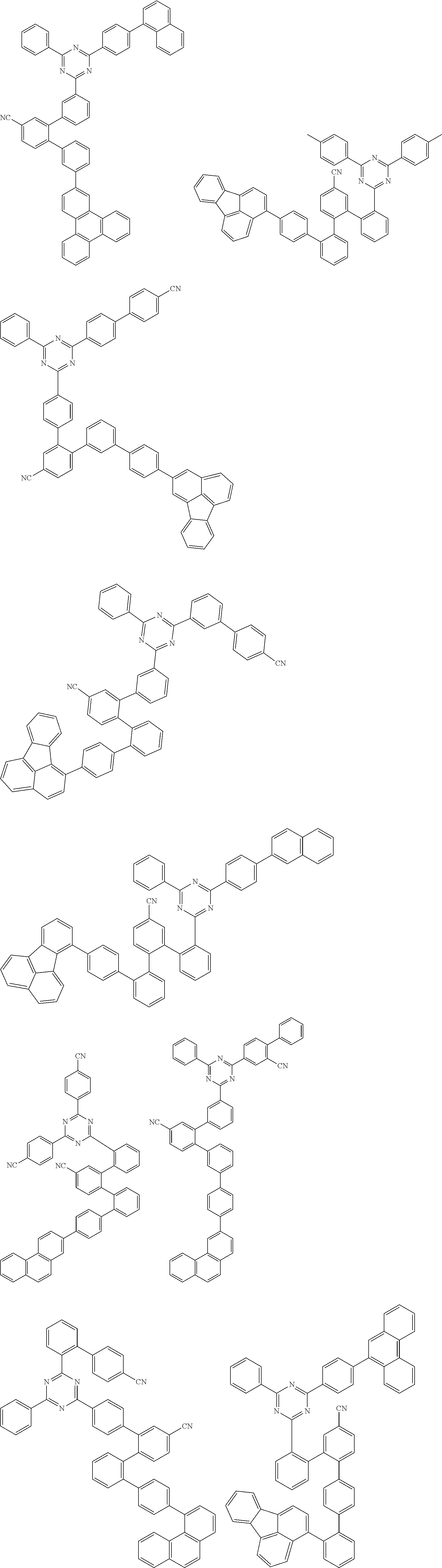

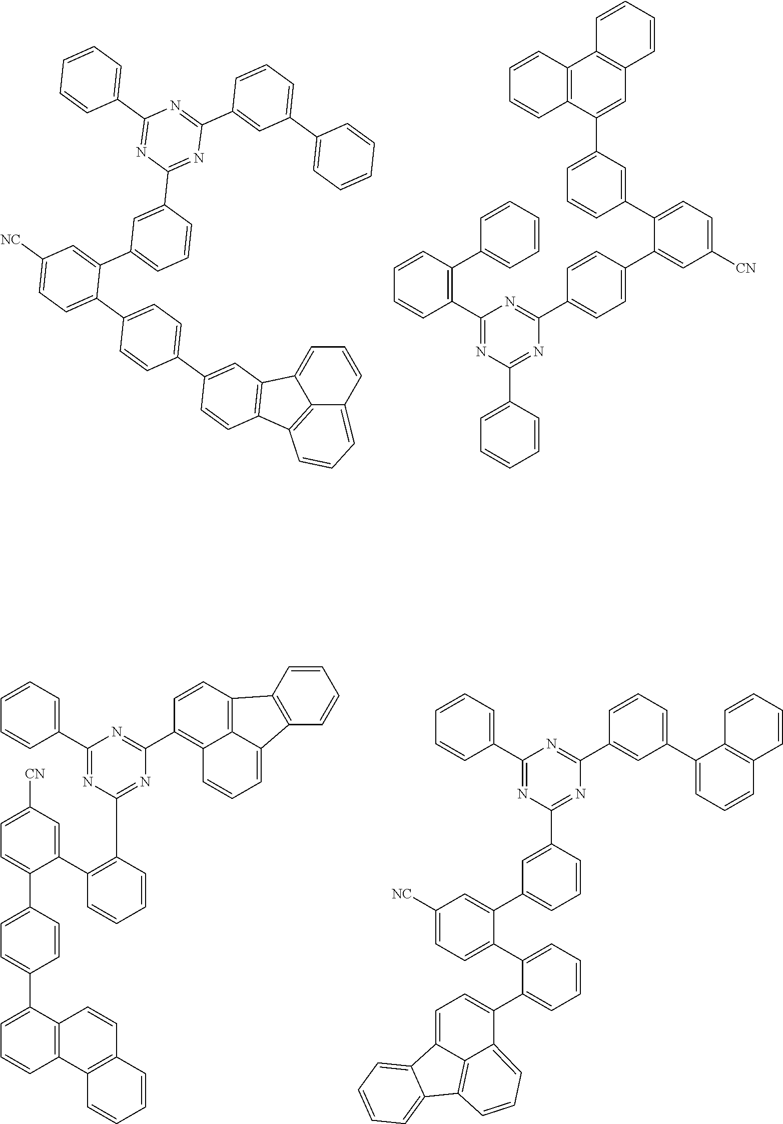 [SMILES: N#Cc1ccc(-c2ccc(-c3ccc4c(c3)-c3cccc5cccc-4c35)cc2)c(-c2cccc(-c3nc(-c4ccccc4)nc(-c4cccc(-c5ccccc5)c4)n3)c2)c1 ; N#Cc1ccc(-c2cccc(-c3cc4ccccc4c4ccccc34)c2)c(-c2ccc(-c3nc(-c4ccccc4)nc(-c4ccccc4-c4ccccc4)n3)cc2)c1 ; N#Cc1ccc(-c2ccc(-c3cccc4c3ccc3ccccc34)cc2)c(-c2ccccc2-c2nc(-c3ccccc3)nc(-c3ccc4c5c(cccc35)-c3ccccc3-4)n2)c1 ; N#Cc1ccc(-c2ccccc2-c2ccc3c4c(cccc24)-c2ccccc2-3)c(-c2cccc(-c3nc(-c4ccccc4)nc(-c4cccc(-c5cccc6ccccc56)c4)n3)c2)c1]

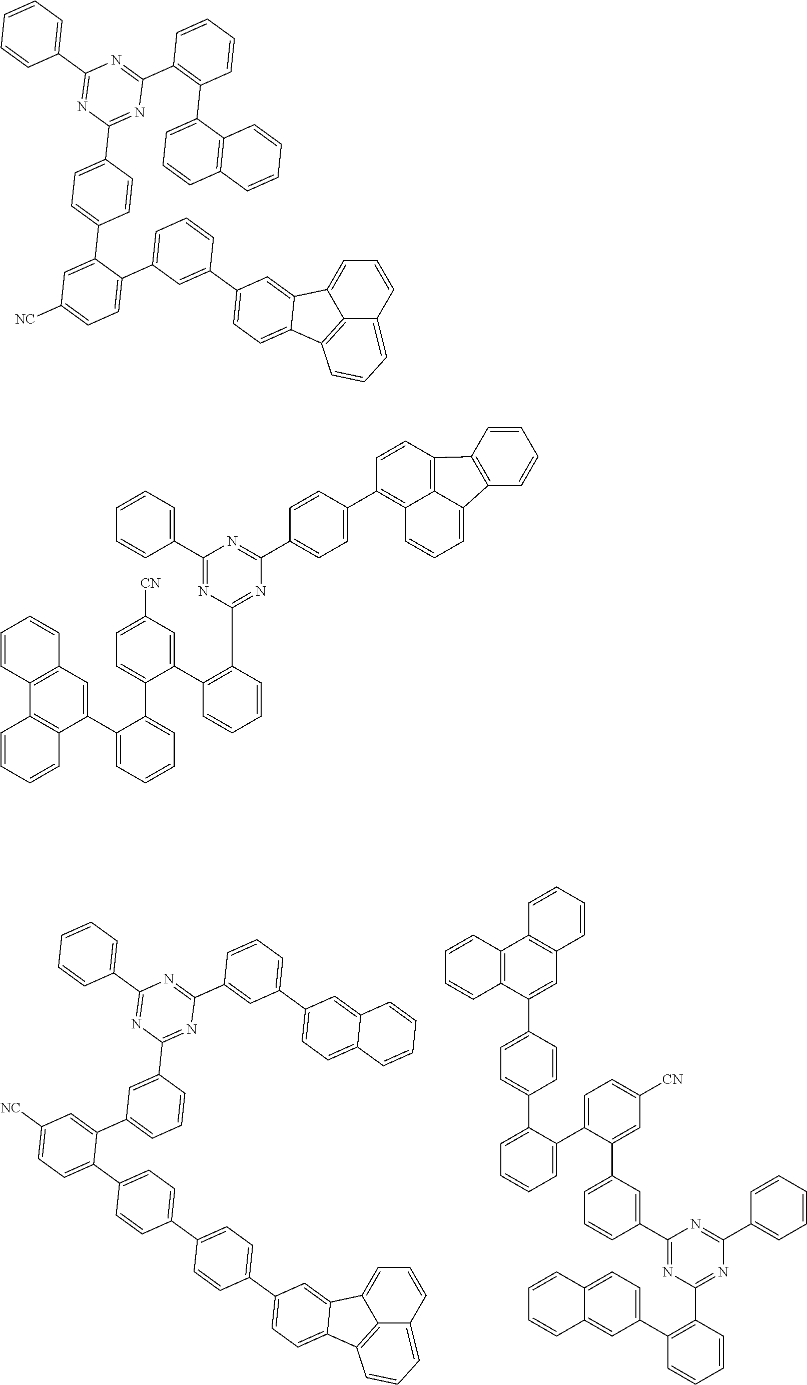 [SMILES: N#Cc1ccc(-c2cccc(-c3ccc4c(c3)-c3cccc5cccc-4c35)c2)c(-c2ccc(-c3nc(-c4ccccc4)nc(-c4ccccc4-c4cccc5ccccc45)n3)cc2)c1 ; N#Cc1ccc(-c2ccccc2-c2cc3ccccc3c3ccccc23)c(-c2ccccc2-c2nc(-c3ccccc3)nc(-c3ccc(-c4ccc5c6c(cccc46)-c4ccccc4-5)cc3)n2)c1 ; N#Cc1ccc(-c2ccc(-c3ccc(-c4ccc5c(c4)-c4cccc6cccc-5c46)cc3)cc2)c(-c2cccc(-c3nc(-c4ccccc4)nc(-c4cccc(-c5ccc6ccccc6c5)c4)n3)c2)c1 ; N#Cc1ccc(-c2ccccc2-c2ccc(-c3cc4ccccc4c4ccccc34)cc2)c(-c2cccc(-c3nc(-c4ccccc4)nc(-c4ccccc4-c4ccc5ccccc5c4)n3)c2)c1]

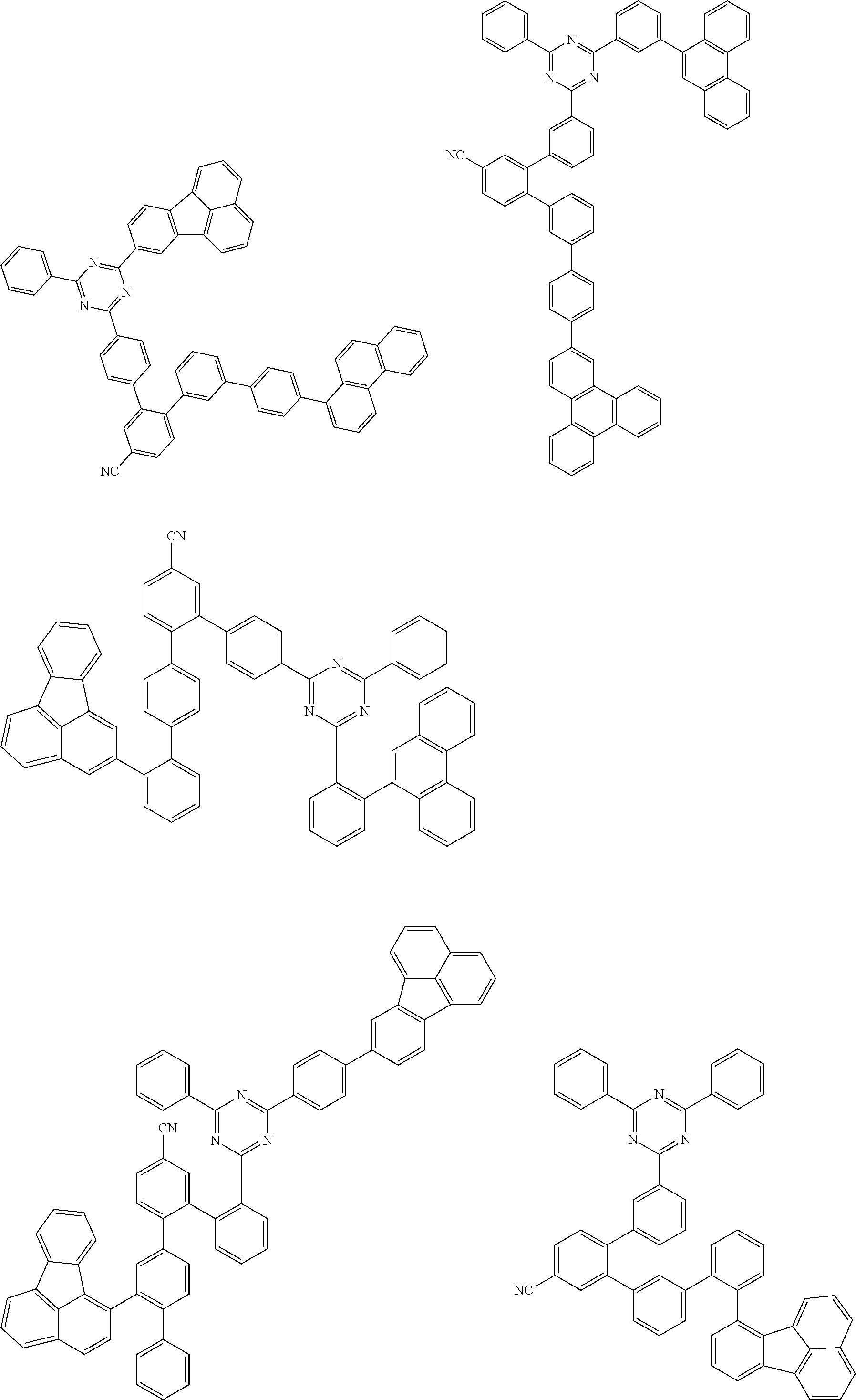

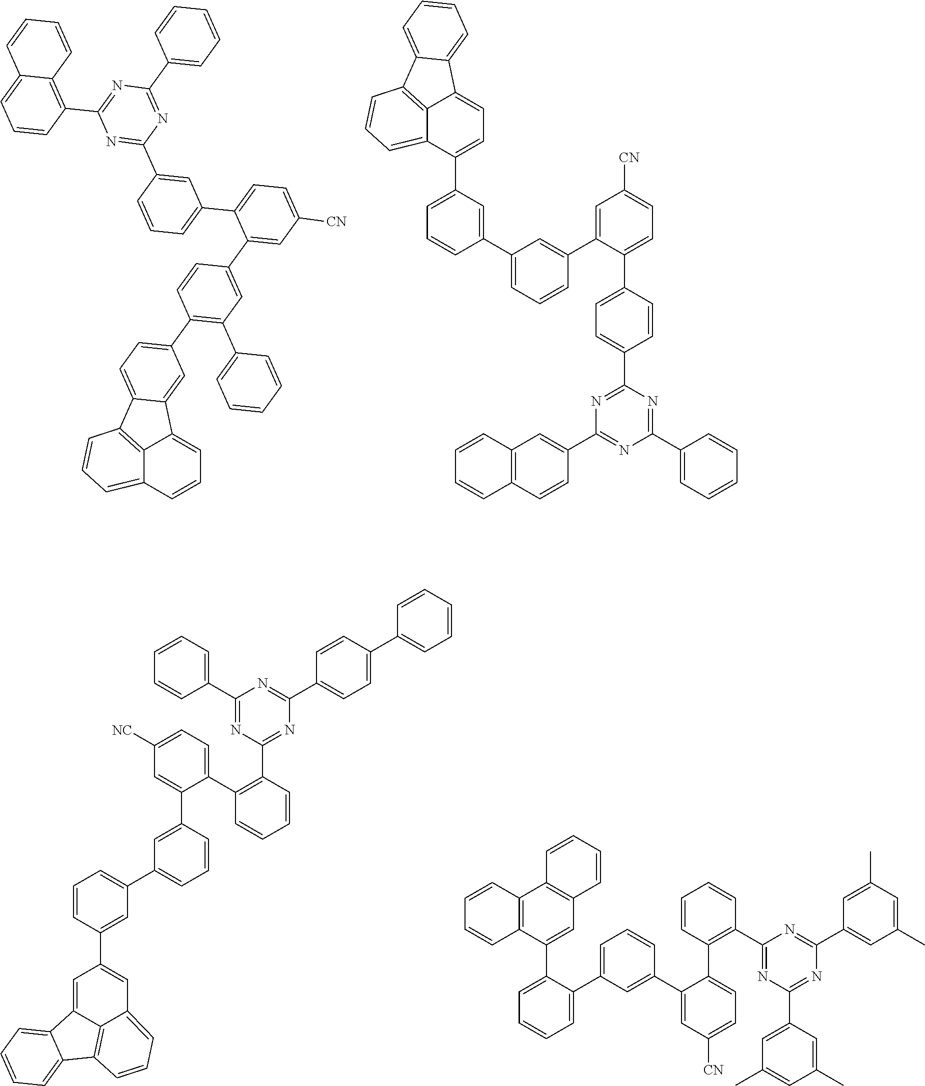 [SMILES: N#Cc1ccc(-c2cccc(-c3nc(-c4ccccc4)nc(-c4cccc5ccccc45)n3)c2)c(-c2ccc(-c3ccc4c(c3)-c3cccc5cccc-4c35)c(-c3ccccc3)c2)c1 ; N#Cc1ccc(-c2ccc(-c3nc(-c4ccccc4)nc(-c4ccc5ccccc5c4)n3)cc2)c(-c2cccc(-c3cccc(-c4ccc5c6c(cccc46)-c4ccccc4-5)c3)c2)c1 ; N#Cc1ccc(-c2ccccc2-c2nc(-c3ccccc3)nc(-c3ccc(-c4ccccc4)cc3)n2)c(-c2cccc(-c3cccc(-c4cc5c6c(cccc6c4)-c4ccccc4-5)c3)c2)c1 ; Cc1cc(C)cc(-c2nc(-c3cc(C)cc(C)c3)nc(-c3ccccc3-c3ccc(C#N)cc3-c3cccc(-c4ccccc4-c4cc5ccccc5c5ccccc45)c3)n2)c1]

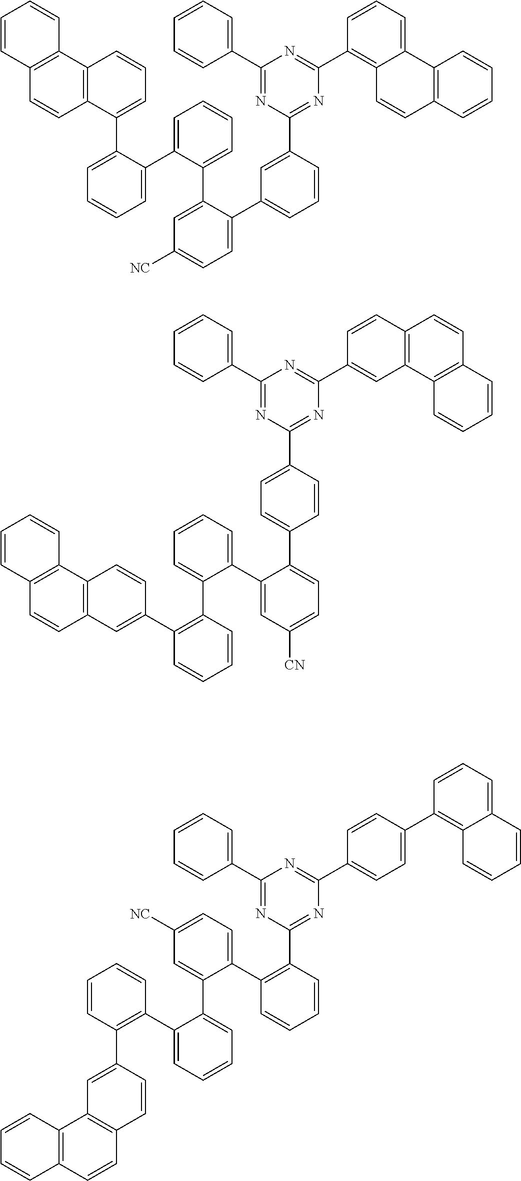 [SMILES: N#Cc1ccc(-c2cccc(-c3nc(-c4ccccc4)nc(-c4cccc5c4ccc4ccccc45)n3)c2)c(-c2ccccc2-c2ccccc2-c2cccc3c2ccc2ccccc23)c1 ; N#Cc1ccc(-c2ccc(-c3nc(-c4ccccc4)nc(-c4ccc5ccc6ccccc6c5c4)n3)cc2)c(-c2ccccc2-c2ccccc2-c2ccc3c(ccc4ccccc43)c2)c1 ; N#Cc1ccc(-c2ccccc2-c2nc(-c3ccccc3)nc(-c3ccc(-c4cccc5ccccc45)cc3)n2)c(-c2ccccc2-c2ccccc2-c2ccc3ccc4ccccc4c3c2)c1]

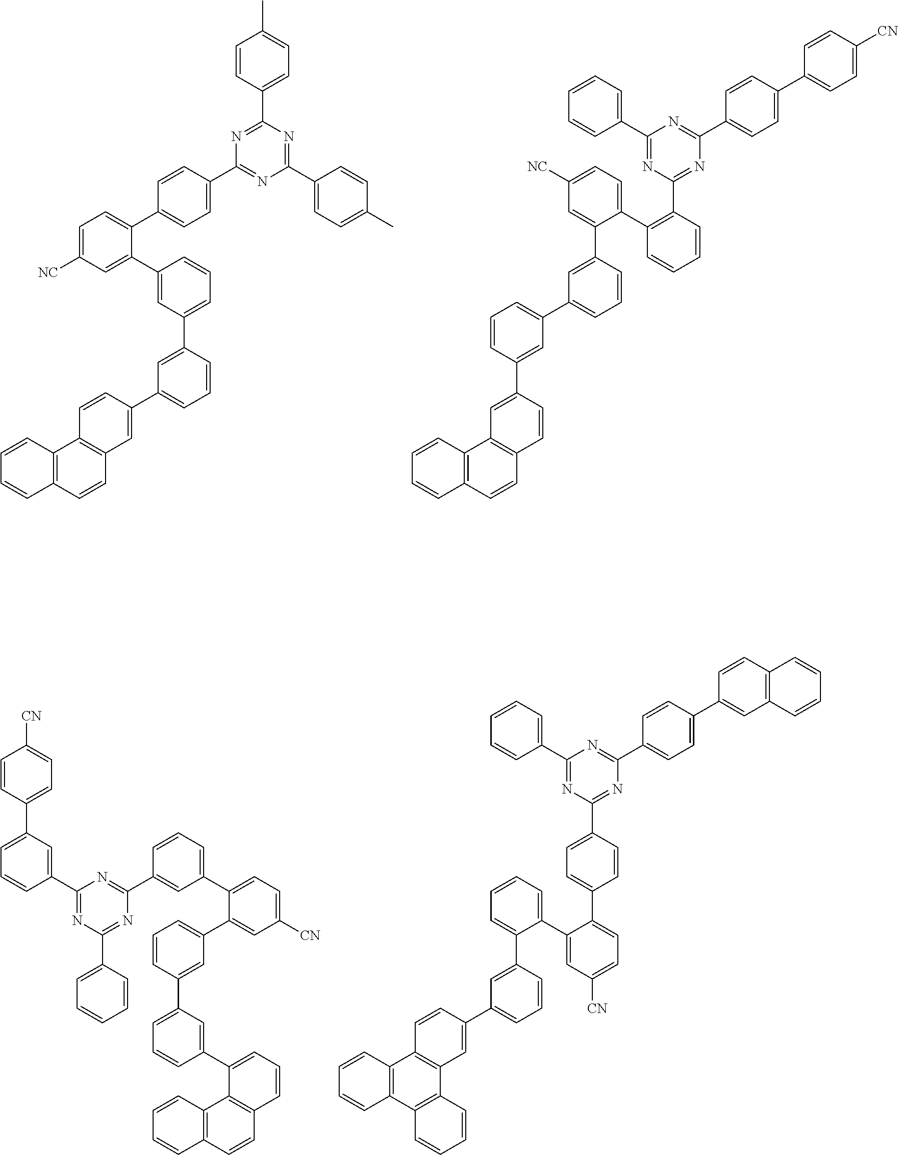 [SMILES: Cc1ccc(-c2nc(-c3ccc(C)cc3)nc(-c3ccc(-c4ccc(C#N)cc4-c4cccc(-c5cccc(-c6ccc7c(ccc8ccccc87)c6)c5)c4)cc3)n2)cc1 ; N#Cc1ccc(-c2ccc(-c3nc(-c4ccccc4)nc(-c4ccccc4-c4ccc(C#N)cc4-c4cccc(-c5cccc(-c6ccc7ccc8ccccc8c7c6)c5)c4)n3)cc2)cc1 ; N#Cc1ccc(-c2cccc(-c3nc(-c4ccccc4)nc(-c4cccc(-c5ccc(C#N)cc5-c5cccc(-c6cccc(-c7cccc8ccc9ccccc9c78)c6)c5)c4)n3)c2)cc1 ; N#Cc1ccc(-c2ccc(-c3nc(-c4ccccc4)nc(-c4ccc(-c5ccc6ccccc6c5)cc4)n3)cc2)c(-c2ccccc2-c2cccc(-c3ccc4c5ccccc5c5ccccc5c4c3)c2)c1]

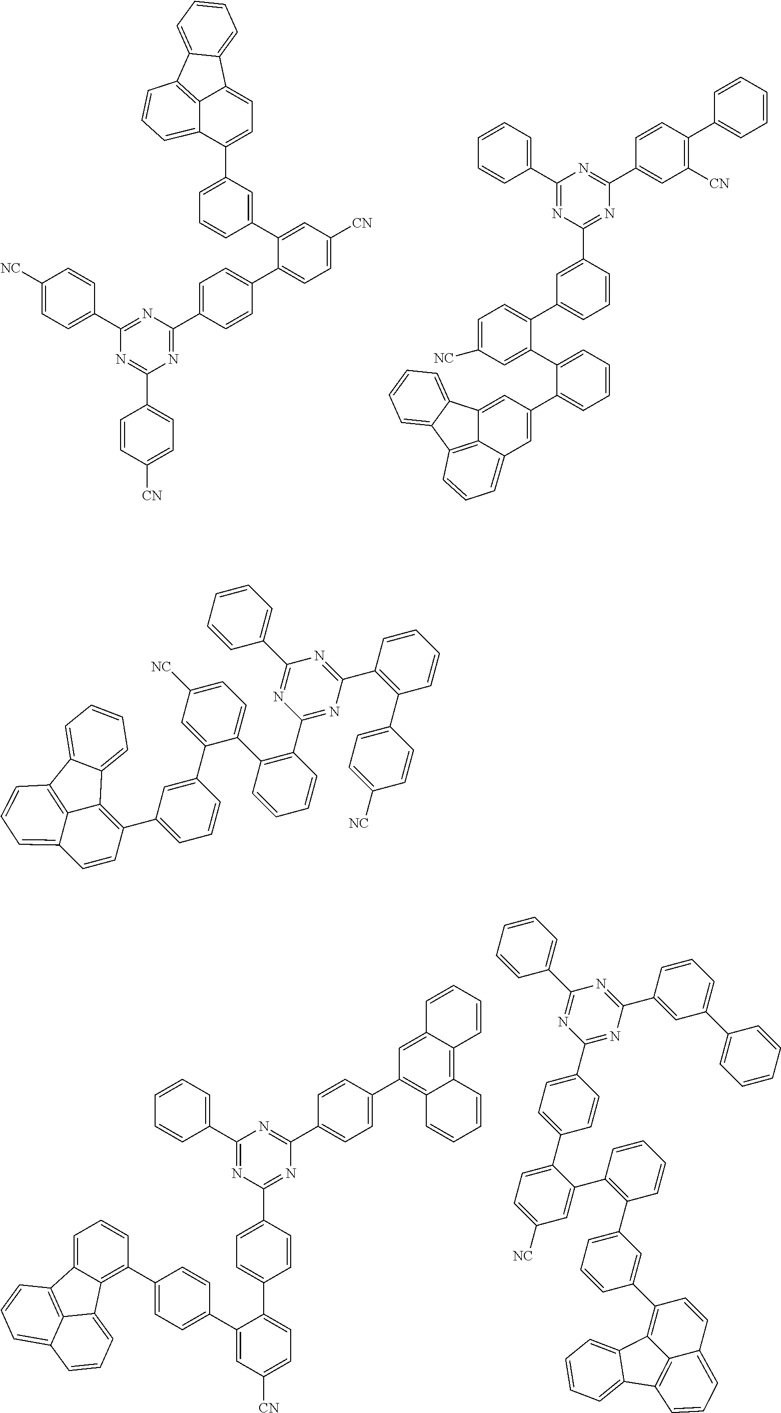

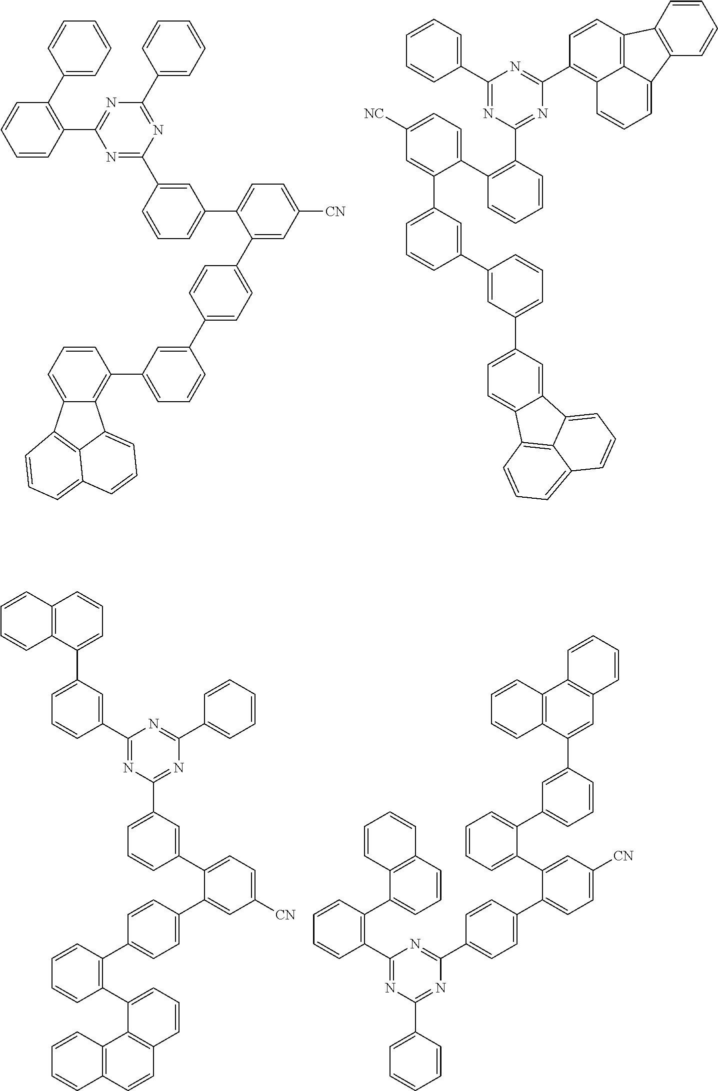

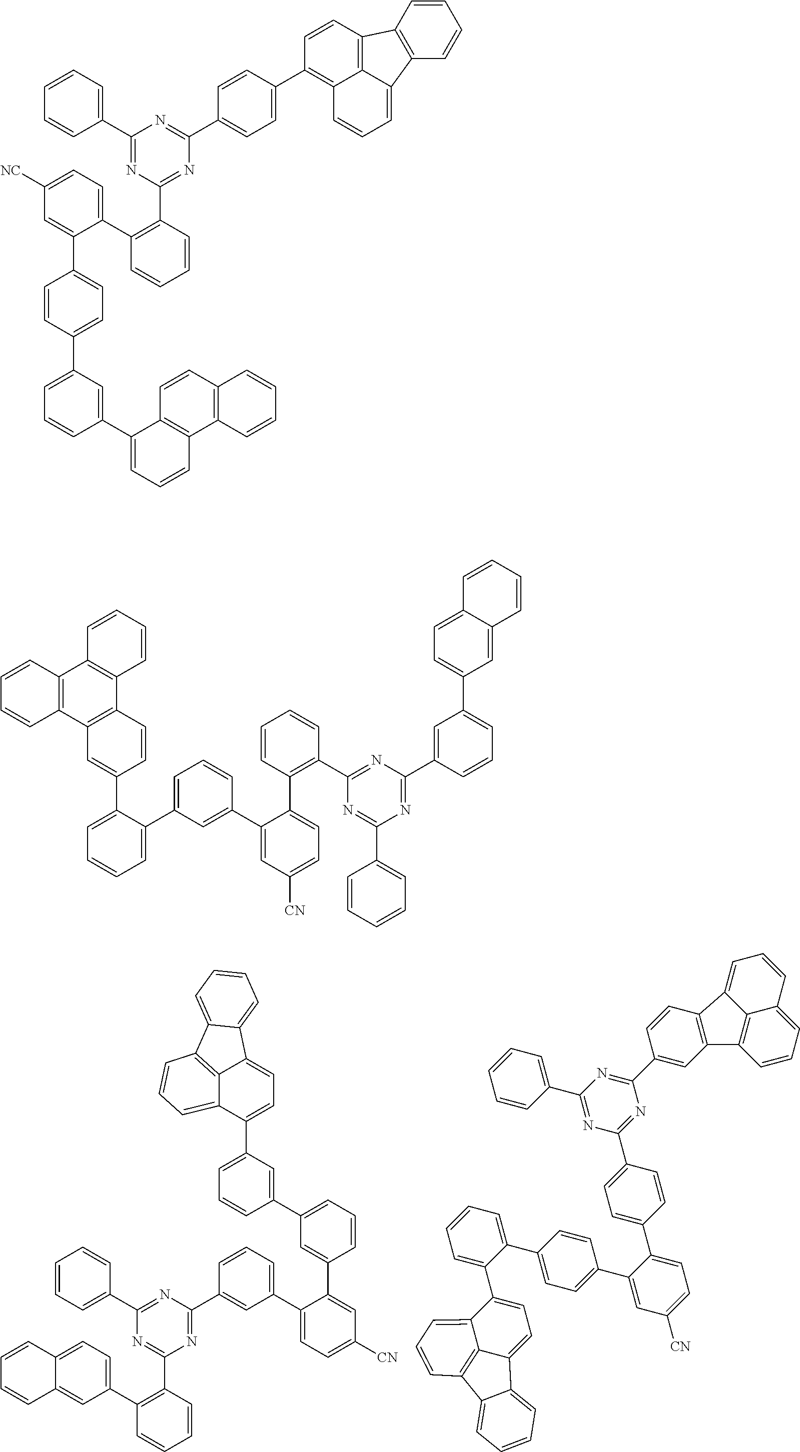

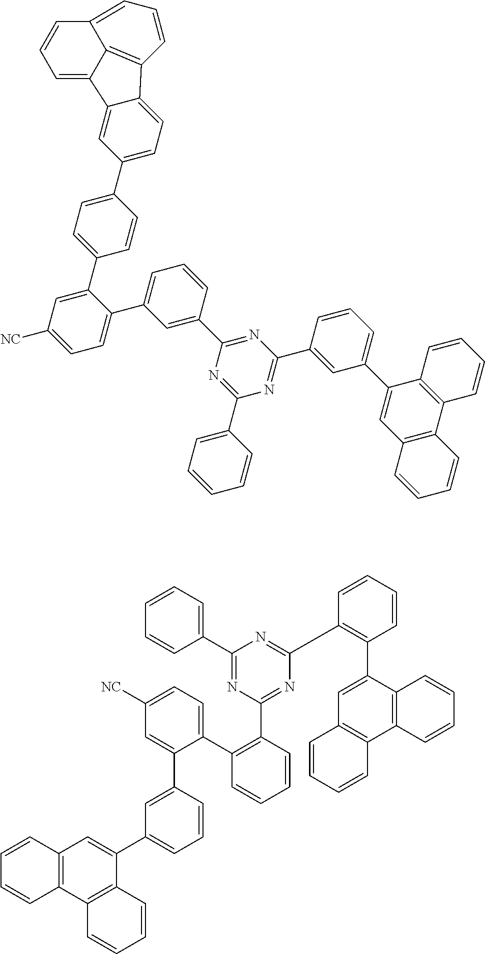 [SMILES: N#Cc1ccc(-c2cccc(-c3nc(-c4ccccc4)nc(-c4cccc(-c5cc6ccccc6c6ccccc56)c4)n3)c2)c(-c2ccc(-c3ccc4c(c3)-c3cccc5cccc-4c35)cc2)c1 ; N#Cc1ccc(-c2ccccc2-c2nc(-c3ccccc3)nc(-c3ccccc3-c3cc4ccccc4c4ccccc34)n2)c(-c2cccc(-c3cc4ccccc4c4ccccc34)c2)c1]

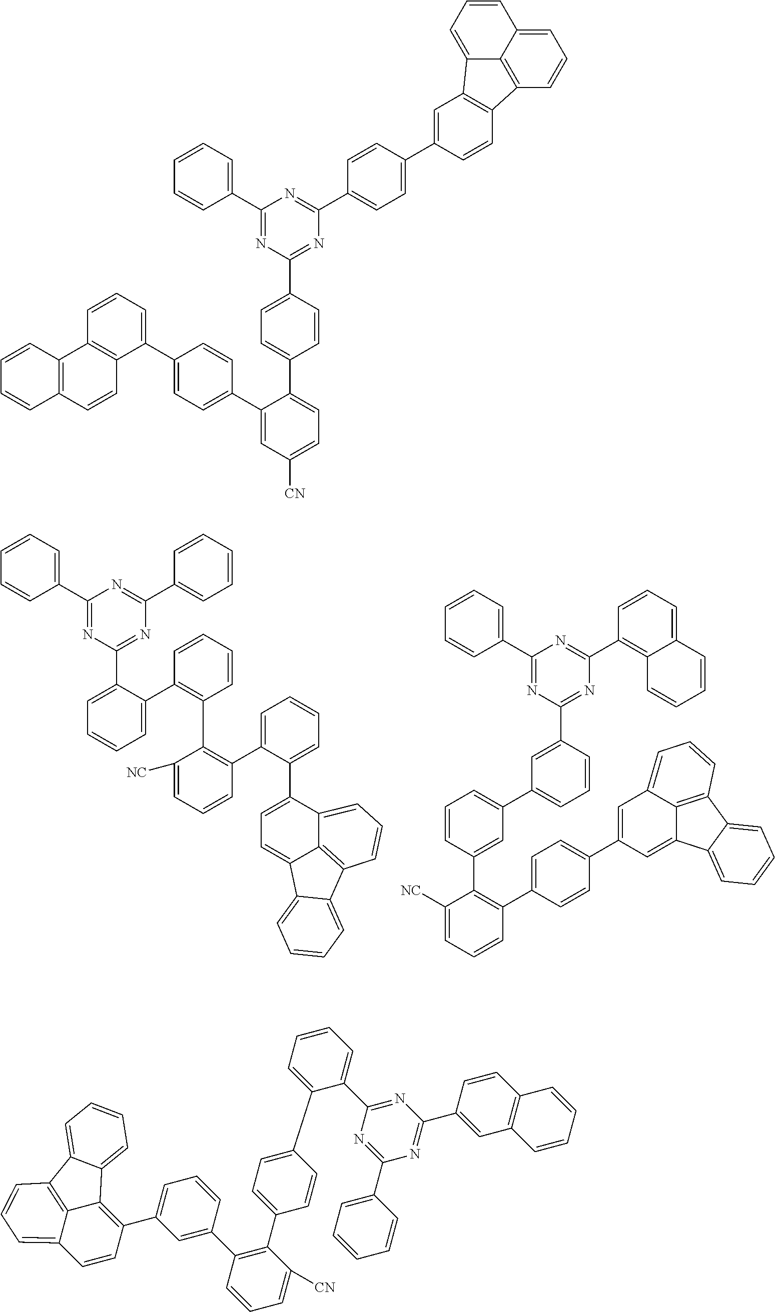 [SMILES: N#Cc1ccc(-c2ccc(-c3nc(-c4ccccc4)nc(-c4ccc(-c5ccc6c(c5)-c5cccc7cccc-6c57)cc4)n3)cc2)c(-c2ccc(-c3cccc4c3ccc3ccccc34)cc2)c1 ; N#Cc1cccc(-c2ccccc2-c2ccc3c4c(cccc24)-c2ccccc2-3)c1-c1ccccc1-c1ccccc1-c1nc(-c2ccccc2)nc(-c2ccccc2)n1 ; N#Cc1cccc(-c2ccc(-c3cc4c5c(cccc5c3)-c3ccccc3-4)cc2)c1-c1cccc(-c2cccc(-c3nc(-c4ccccc4)nc(-c4cccc5ccccc45)n3)c2)c1 ; N#Cc1cccc(-c2cccc(-c3ccc4cccc5c4c3-c3ccccc3-5)c2)c1-c1ccc(-c2ccccc2-c2nc(-c3ccccc3)nc(-c3ccc4ccccc4c3)n2)cc1]

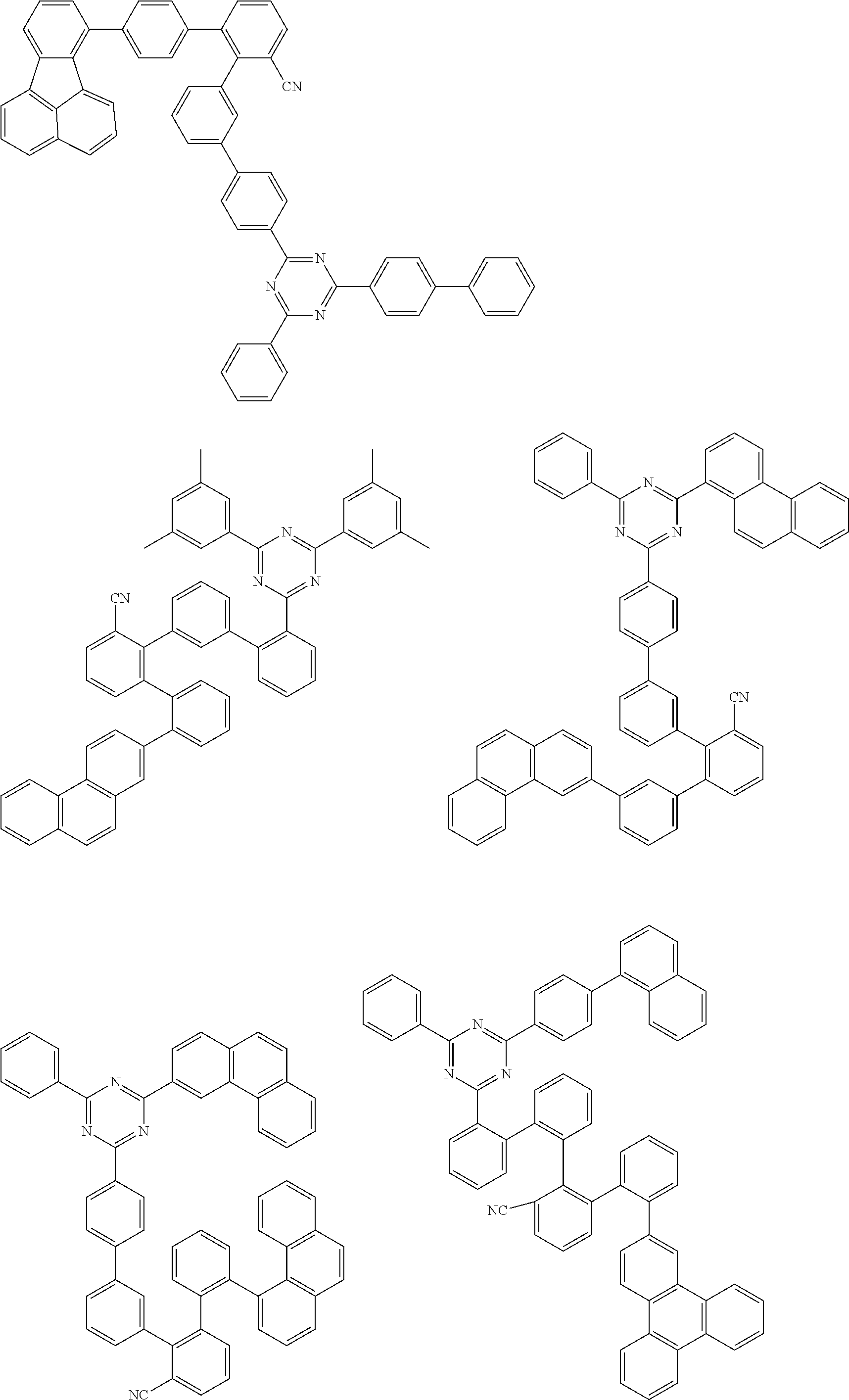

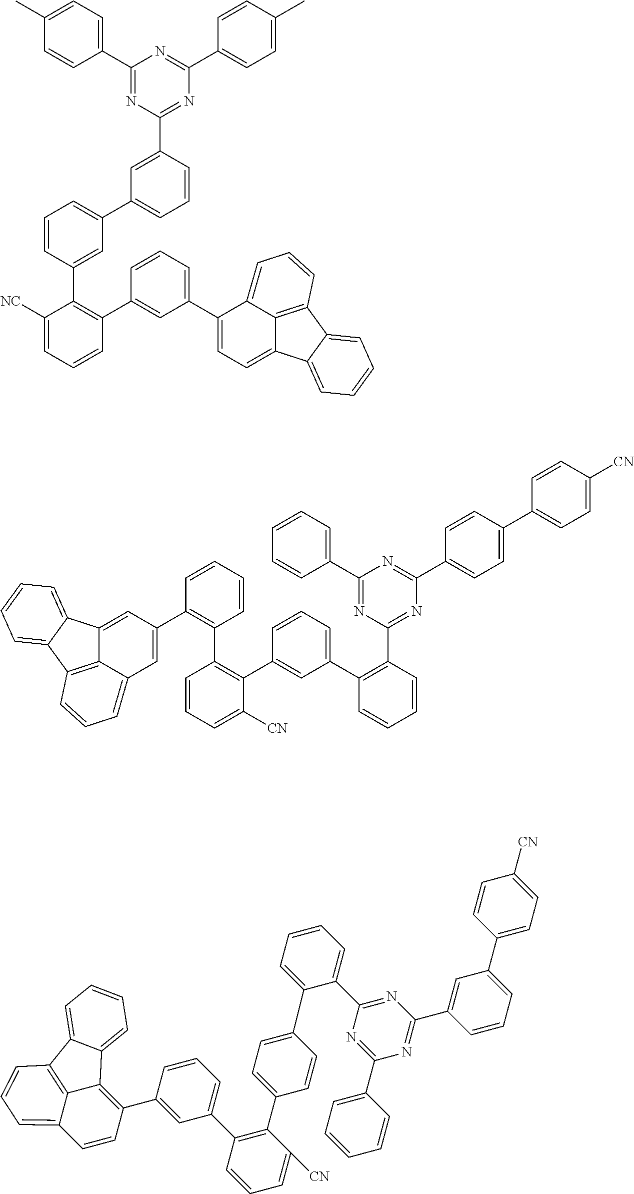 [SMILES: Cc1ccc(-c2nc(-c3ccc(C)cc3)nc(-c3cccc(-c4cccc(-c5c(C#N)cccc5-c5cccc(-c6ccc7c8c(cccc68)-c6ccccc6-7)c5)c4)c3)n2)cc1 ; N#Cc1ccc(-c2ccc(-c3nc(-c4ccccc4)nc(-c4ccccc4-c4cccc(-c5c(C#N)cccc5-c5ccccc5-c5cc6c7c(cccc7c5)-c5ccccc5-6)c4)n3)cc2)cc1 ; N#Cc1ccc(-c2cccc(-c3nc(-c4ccccc4)nc(-c4ccccc4-c4ccc(-c5c(C#N)cccc5-c5cccc(-c6ccc7cccc8c7c6-c6ccccc6-8)c5)cc4)n3)c2)cc1]

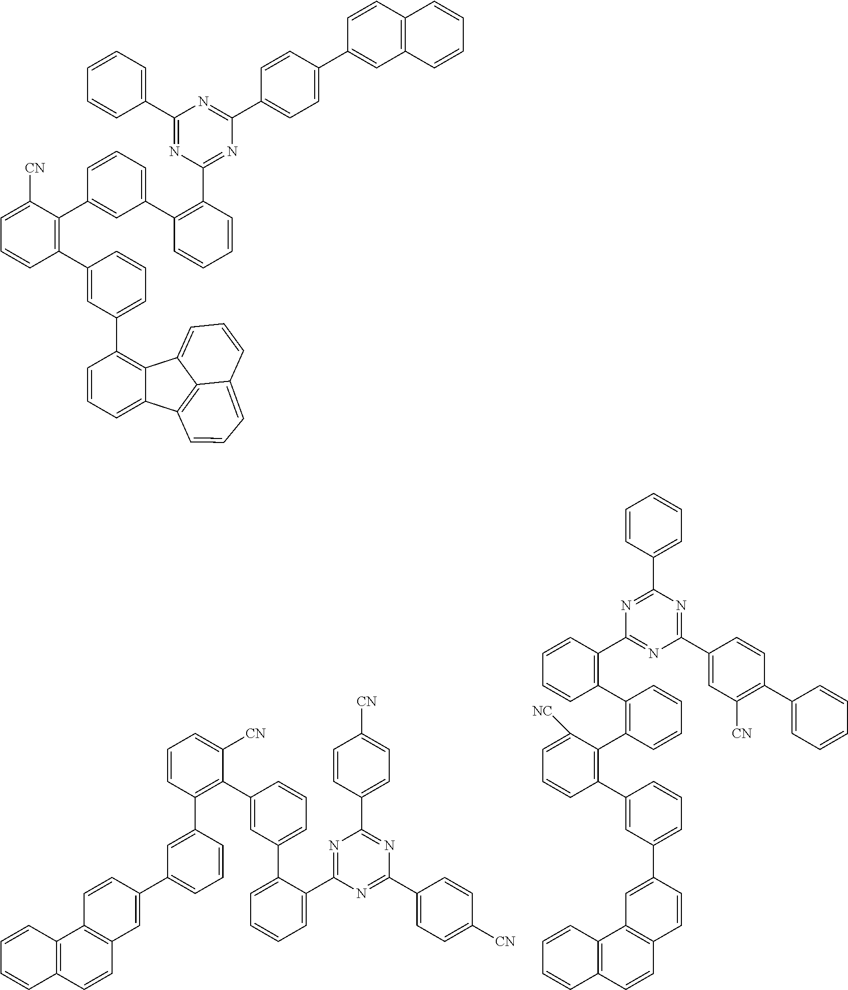

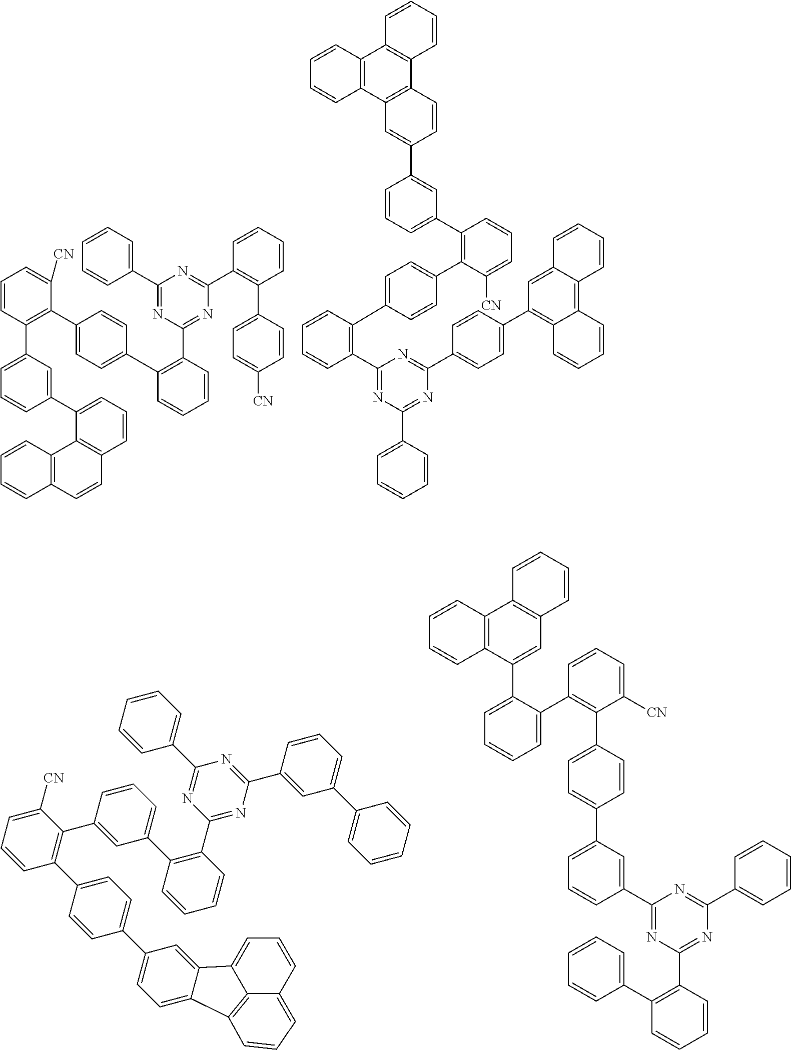

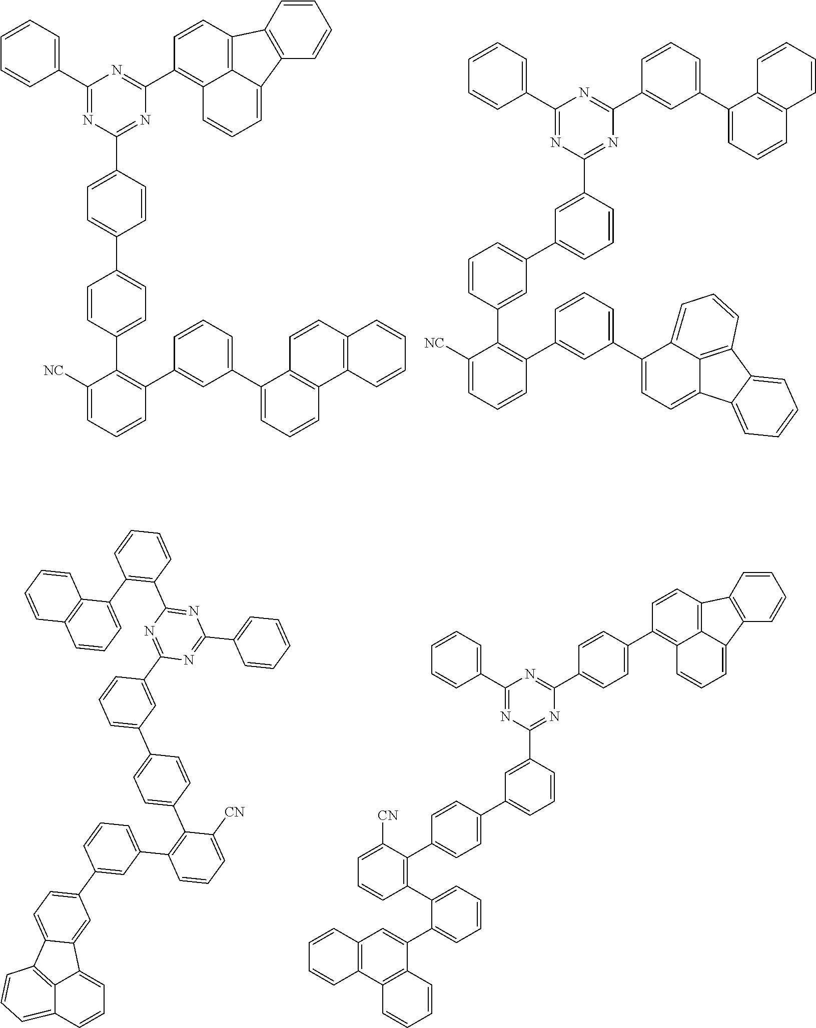

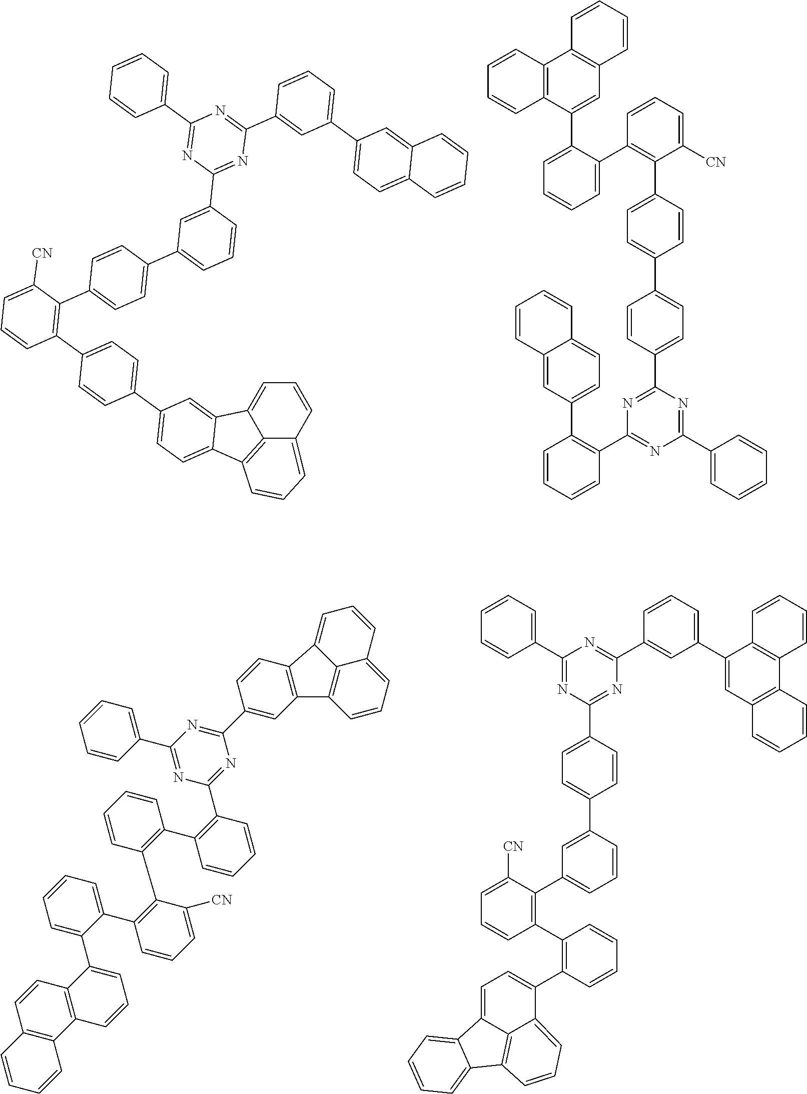 [SMILES: N#Cc1cccc(-c2ccc(-c3ccc4c(c3)-c3cccc5cccc-4c35)cc2)c1-c1ccc(-c2cccc(-c3nc(-c4ccccc4)nc(-c4cccc(-c5ccc6ccccc6c5)c4)n3)c2)cc1 ; N#Cc1cccc(-c2ccccc2-c2cc3ccccc3c3ccccc23)c1-c1ccc(-c2ccc(-c3nc(-c4ccccc4)nc(-c4ccccc4-c4ccc5ccccc5c4)n3)cc2)cc1 ; N#Cc1cccc(-c2ccccc2-c2cccc3c2ccc2ccccc23)c1-c1ccccc1-c1ccccc1-c1nc(-c2ccccc2)nc(-c2ccc3c(c2)-c2cccc4cccc-3c24)n1 ; N#Cc1cccc(-c2ccccc2-c2ccc3c4c(cccc24)-c2ccccc2-3)c1-c1cccc(-c2ccc(-c3nc(-c4ccccc4)nc(-c4cccc(-c5cc6ccccc6c6ccccc56)c4)n3)cc2)c1]

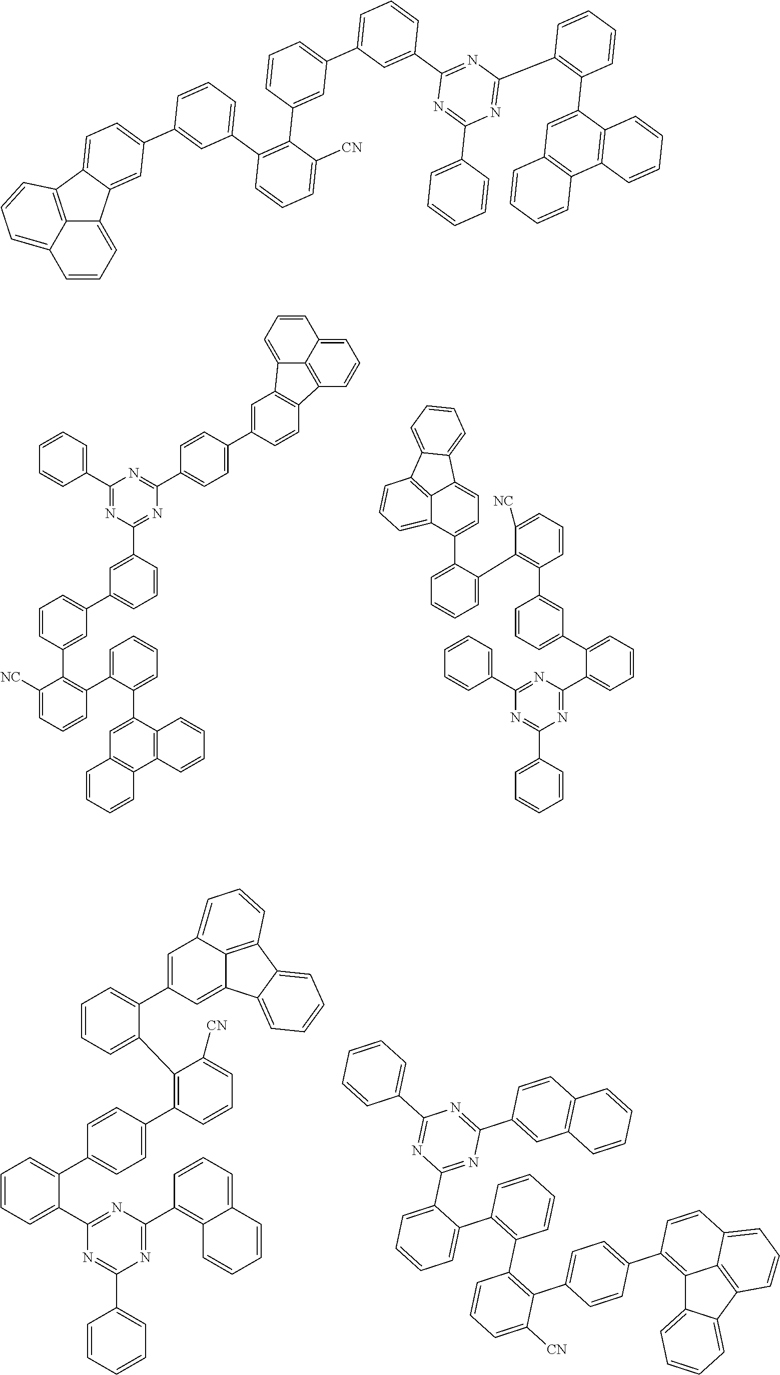

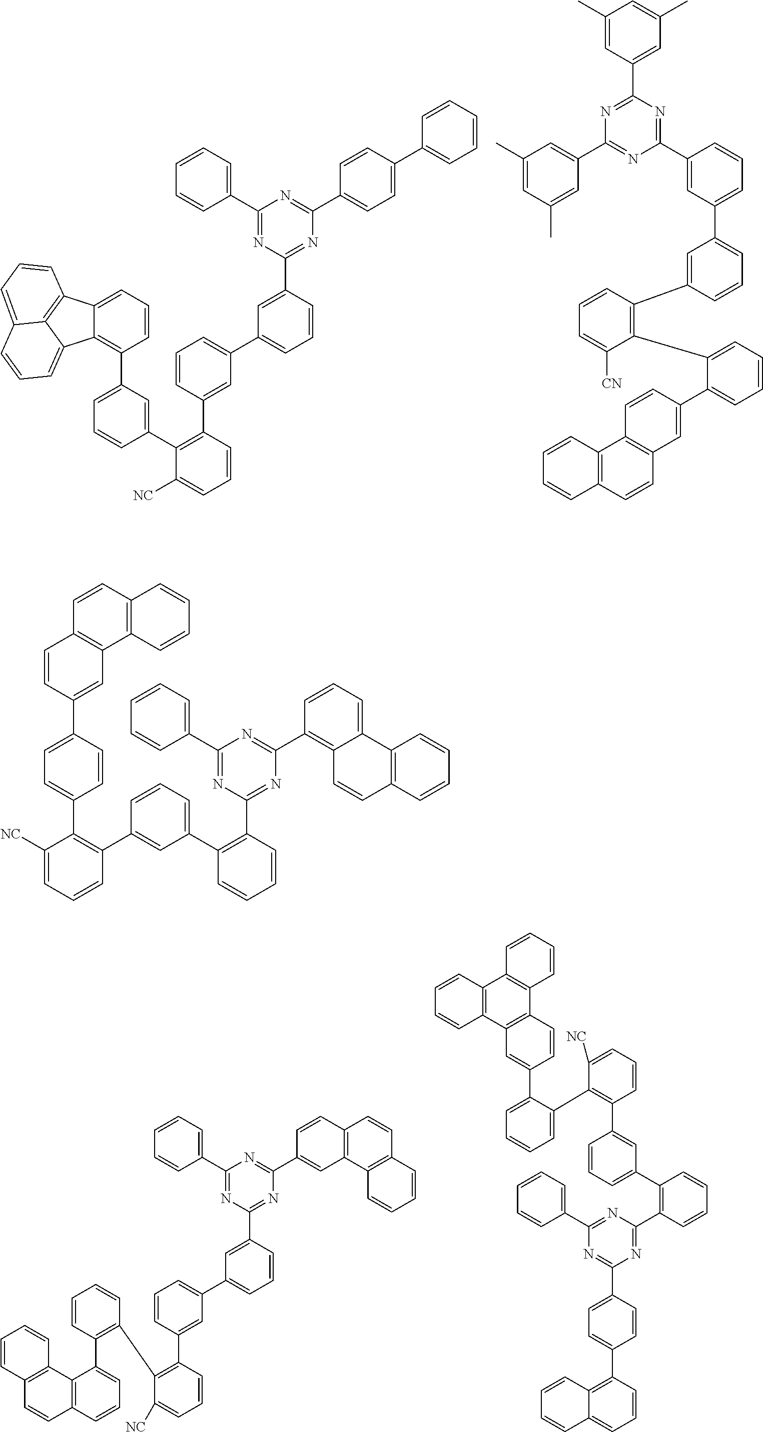

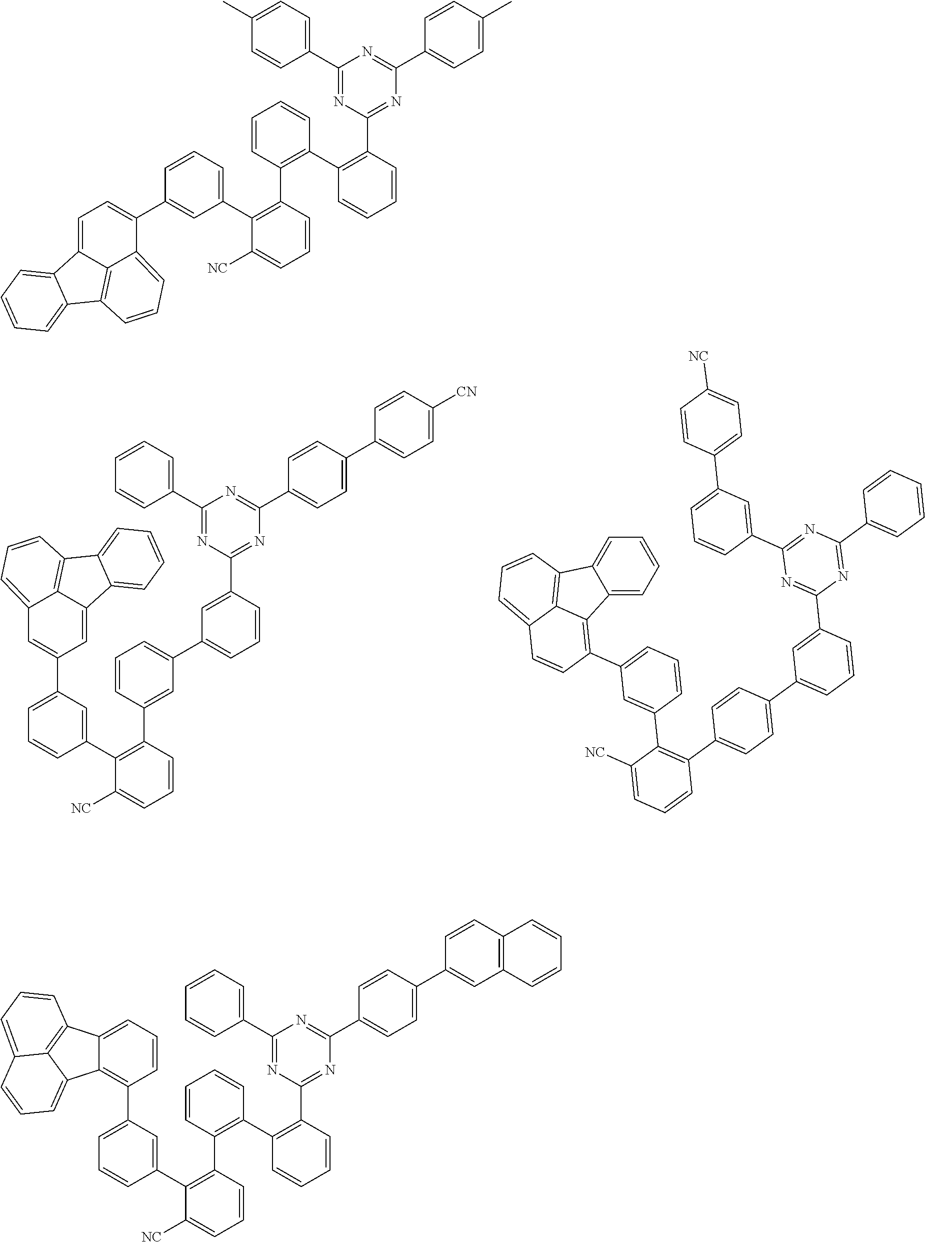 [SMILES: Cc1ccc(-c2nc(-c3ccc(C)cc3)nc(-c3ccccc3-c3ccccc3-c3cccc(C#N)c3-c3cccc(-c4ccc5c6c(cccc46)-c4ccccc4-5)c3)n2)cc1 ; N#Cc1ccc(-c2ccc(-c3nc(-c4ccccc4)nc(-c4cccc(-c5cccc(-c6cccc(C#N)c6-c6cccc(-c7cc8c9c(cccc9c7)-c7ccccc7-8)c6)c5)c4)n3)cc2)cc1 ; N#Cc1ccc(-c2cccc(-c3nc(-c4ccccc4)nc(-c4cccc(-c5ccc(-c6cccc(C#N)c6-c6cccc(-c7ccc8cccc9c8c7-c7ccccc7-9)c6)cc5)c4)n3)c2)cc1 ; N#Cc1cccc(-c2ccccc2-c2ccccc2-c2nc(-c3ccccc3)nc(-c3ccc(-c4ccc5ccccc5c4)cc3)n2)c1-c1cccc(-c2cccc3c2-c2cccc4cccc-3c24)c1]

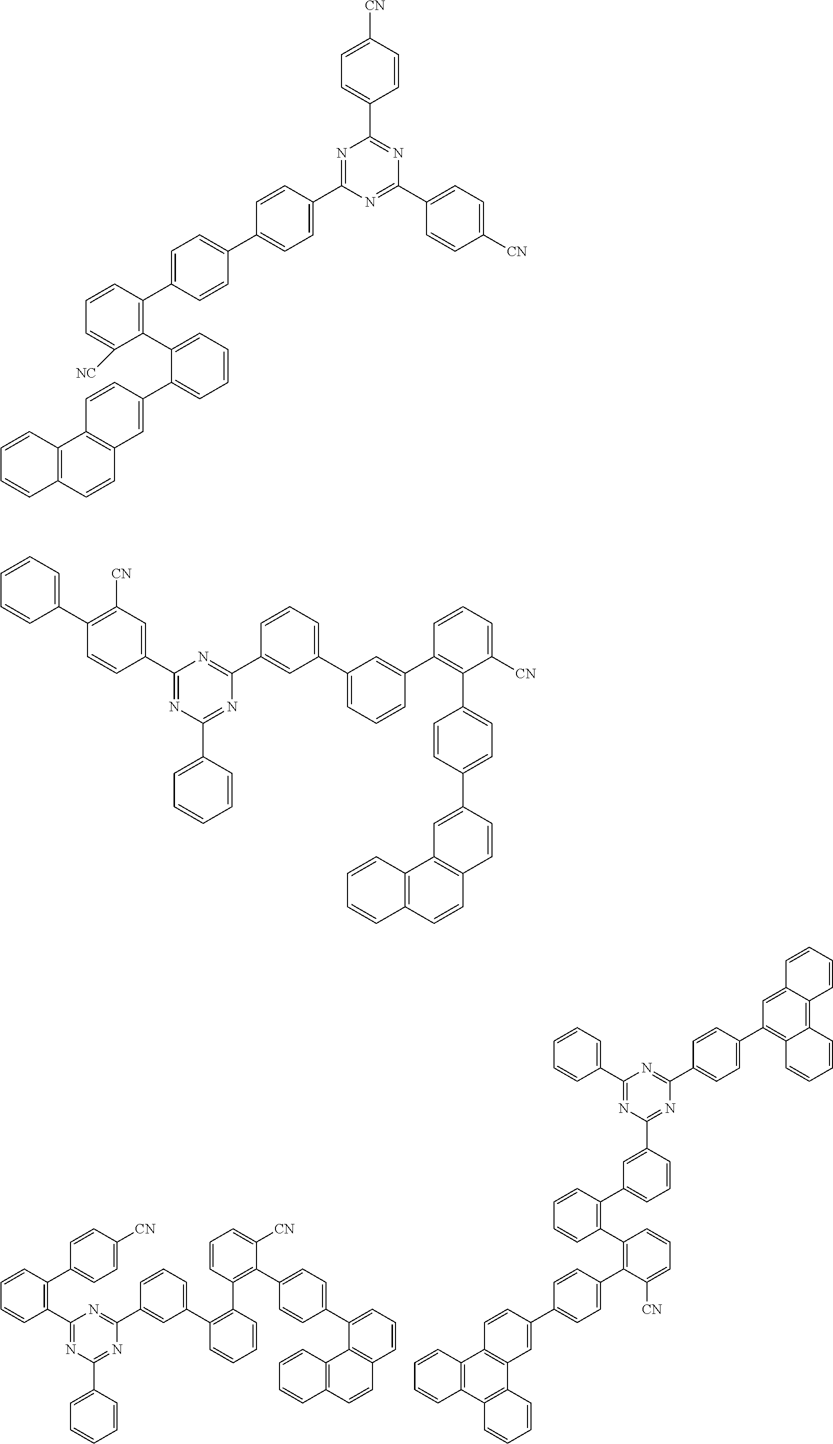

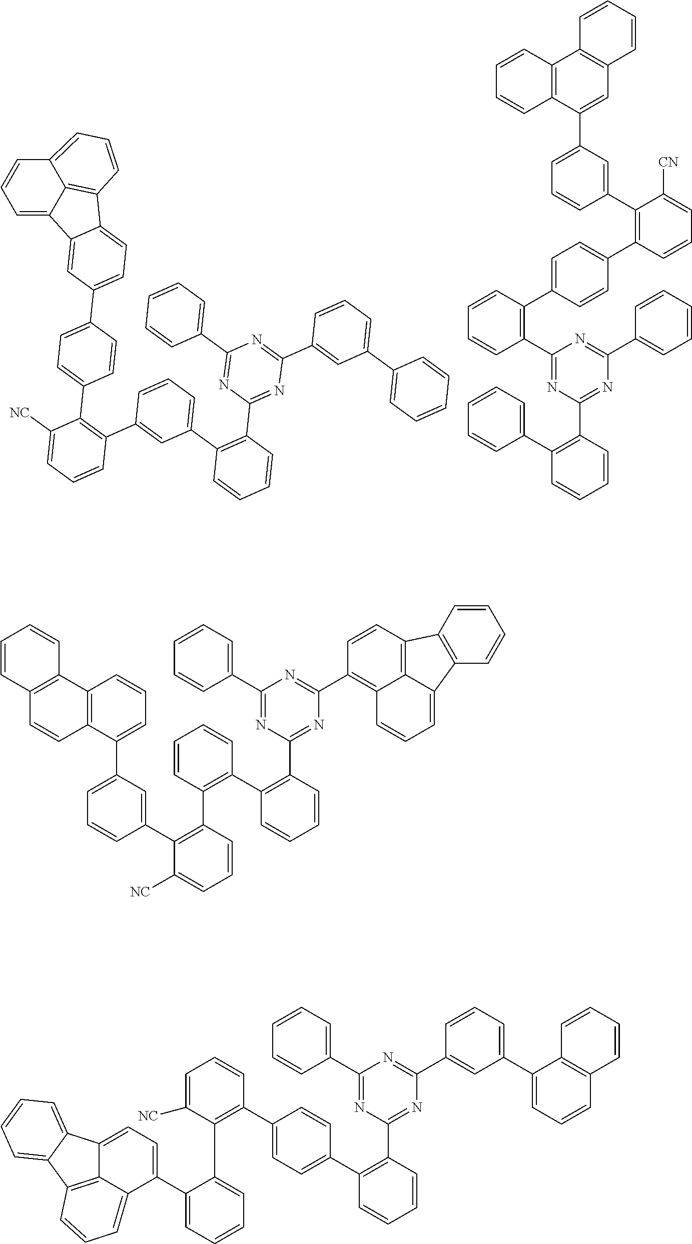

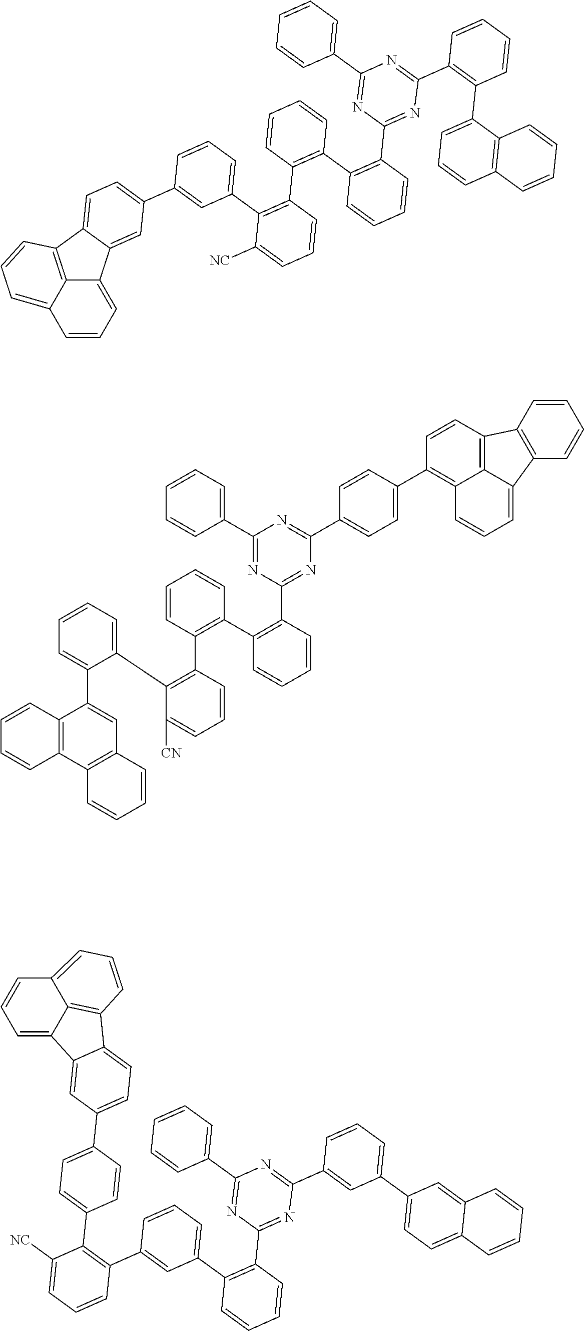 [SMILES: N#Cc1cccc(-c2ccccc2-c2ccccc2-c2nc(-c3ccccc3)nc(-c3ccccc3-c3cccc4ccccc34)n2)c1-c1cccc(-c2ccc3c(c2)-c2cccc4cccc-3c24)c1 ; N#Cc1cccc(-c2ccccc2-c2ccccc2-c2nc(-c3ccccc3)nc(-c3ccc(-c4ccc5c6c(cccc46)-c4ccccc4-5)cc3)n2)c1-c1ccccc1-c1cc2ccccc2c2ccccc12 ; N#Cc1cccc(-c2cccc(-c3ccccc3-c3nc(-c4ccccc4)nc(-c4cccc(-c5ccc6ccccc6c5)c4)n3)c2)c1-c1ccc(-c2ccc3c(c2)-c2cccc4cccc-3c24)cc1]

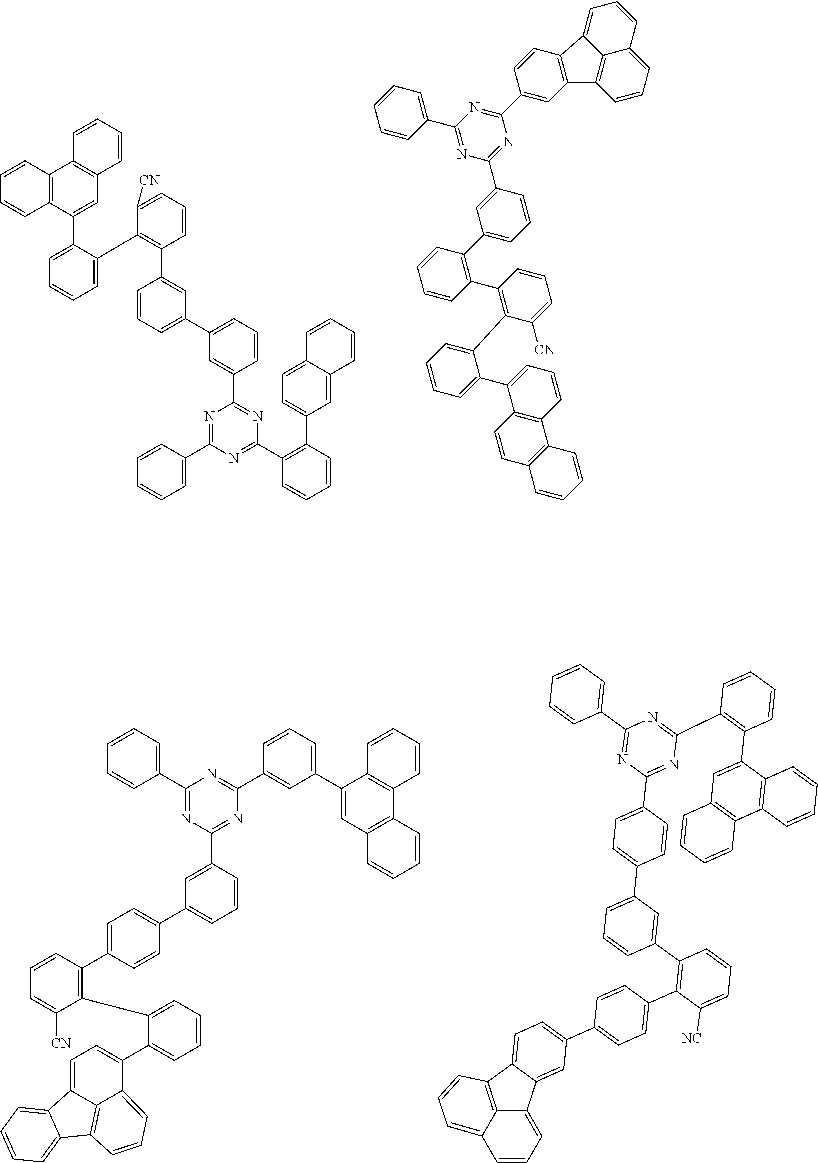 [SMILES: N#Cc1cccc(-c2cccc(-c3cccc(-c4nc(-c5ccccc5)nc(-c5ccccc5-c5ccc6ccccc6c5)n4)c3)c2)c1-c1ccccc1-c1cc2ccccc2c2ccccc12 ; N#Cc1cccc(-c2ccccc2-c2cccc(-c3nc(-c4ccccc4)nc(-c4ccc5c(c4)-c4cccc6cccc-5c46)n3)c2)c1-c1ccccc1-c1cccc2c1ccc1ccccc12 ; N#Cc1cccc(-c2ccc(-c3cccc(-c4nc(-c5ccccc5)nc(-c5cccc(-c6cc7ccccc7c7ccccc67)c5)n4)c3)cc2)c1-c1ccccc1-c1ccc2c3c(cccc13)-c1ccccc1-2 ; N#Cc1cccc(-c2cccc(-c3ccc(-c4nc(-c5ccccc5)nc(-c5ccccc5-c5cc6ccccc6c6ccccc56)n4)cc3)c2)c1-c1ccc(-c2ccc3c(c2)-c2cccc4cccc-3c24)cc1]

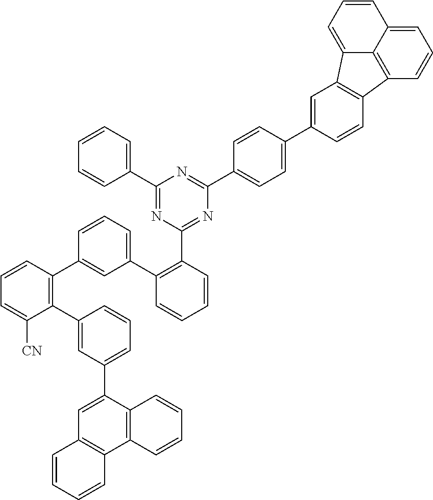 [SMILES: N#Cc1cccc(-c2cccc(-c3ccccc3-c3nc(-c4ccccc4)nc(-c4ccc(-c5ccc6c(c5)-c5cccc7cccc-6c57)cc4)n3)c2)c1-c1cccc(-c2cc3ccccc3c3ccccc23)c1]

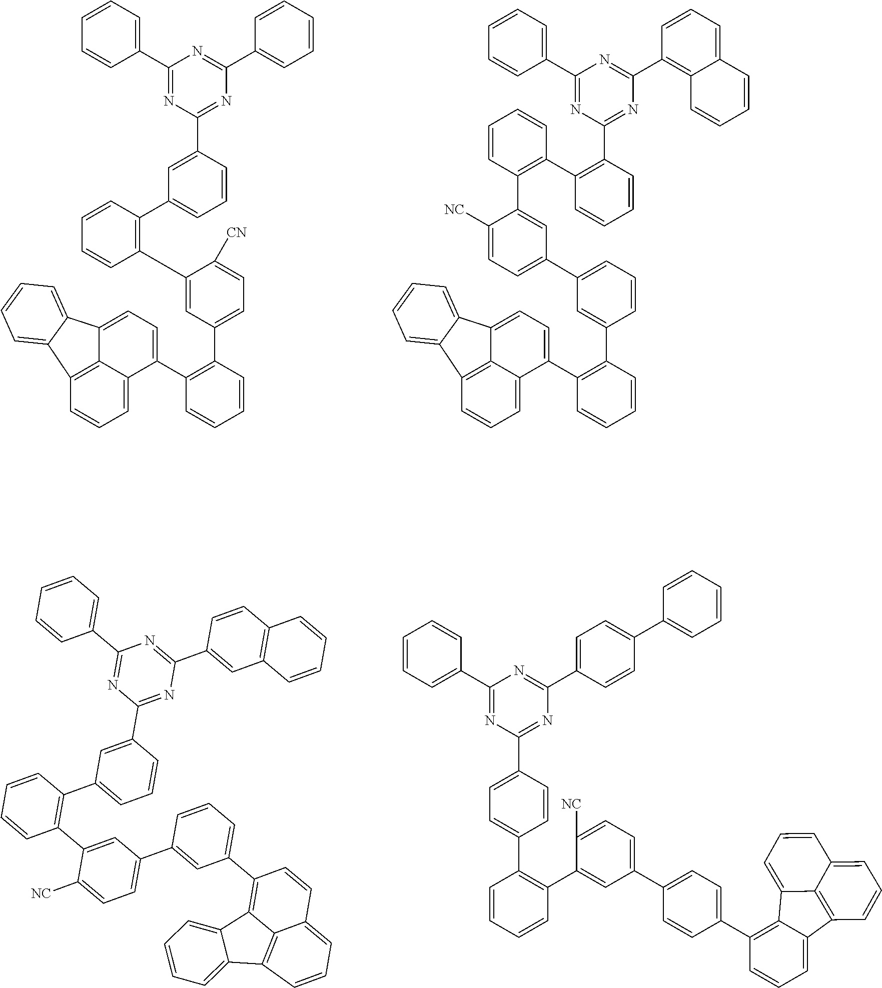

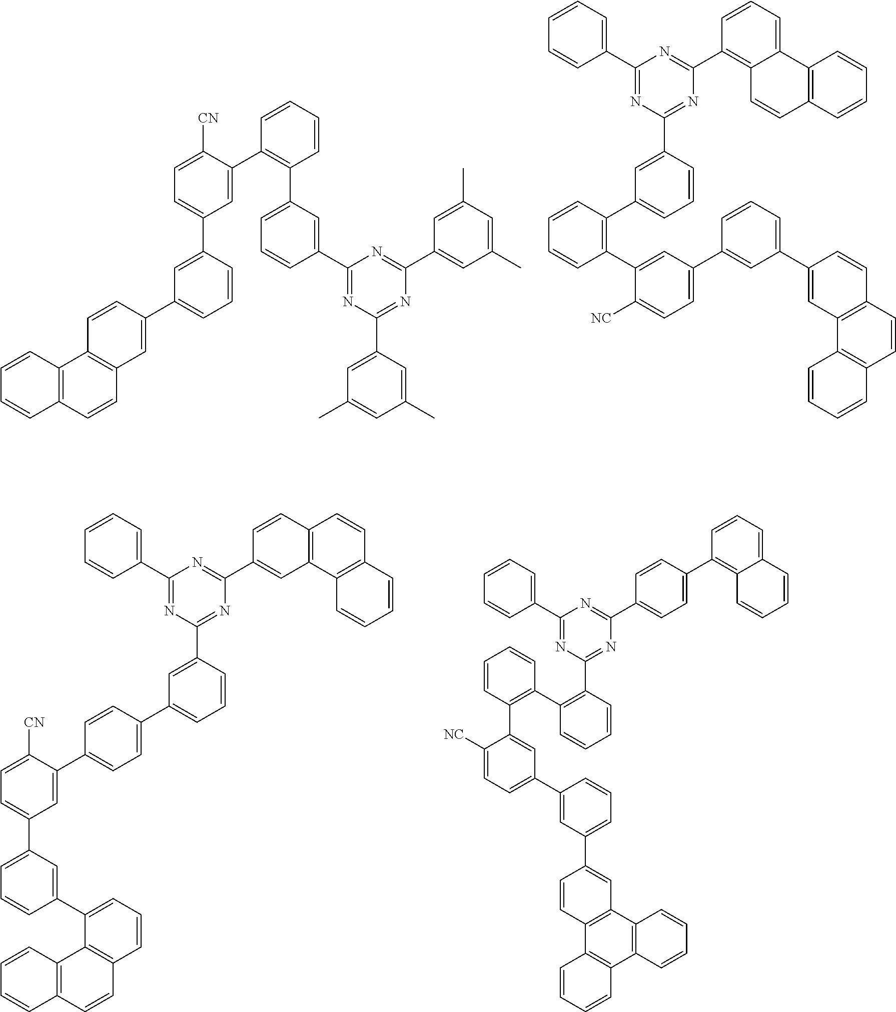

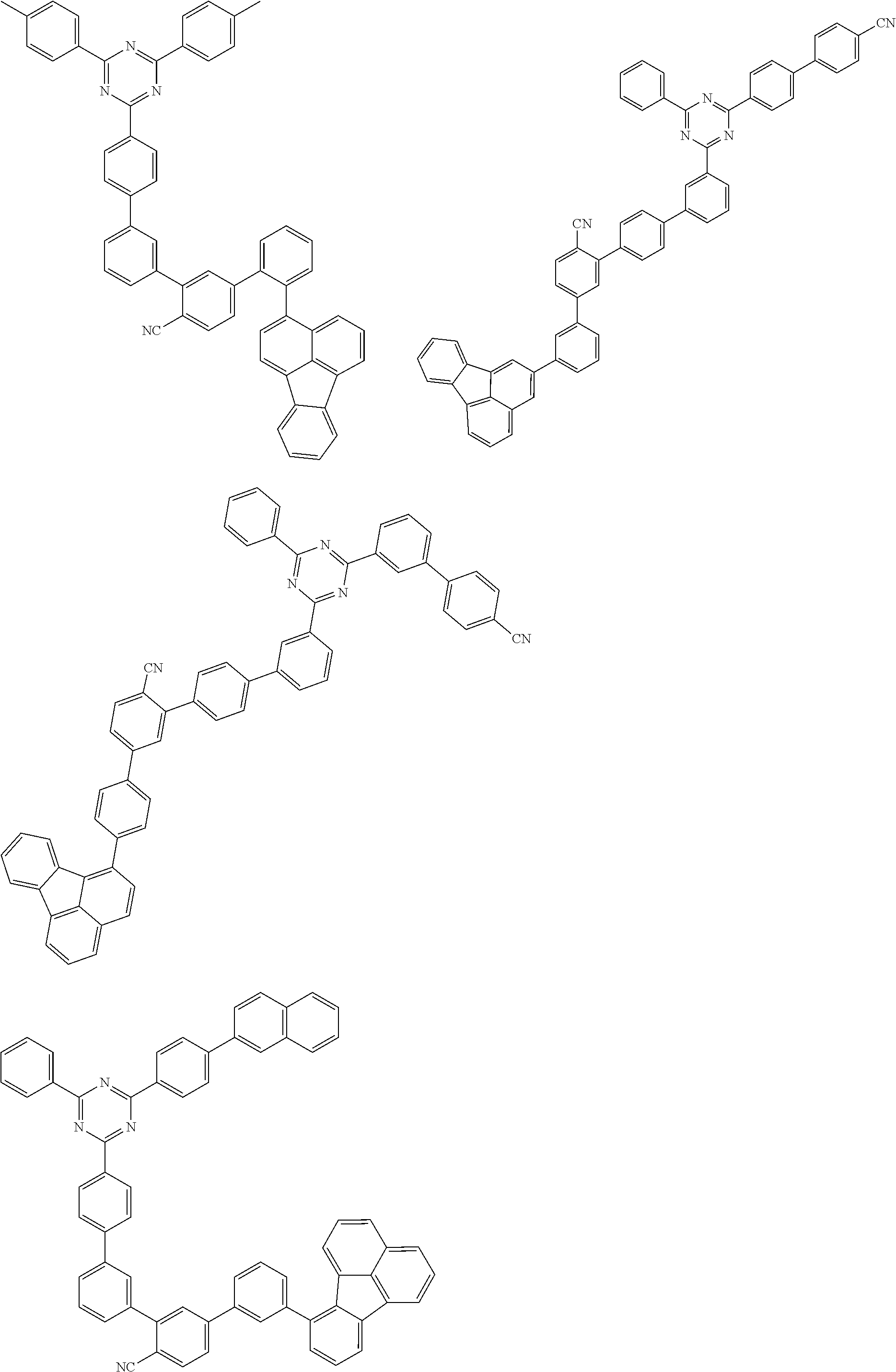 [SMILES: Cc1ccc(-c2nc(-c3ccc(C)cc3)nc(-c3ccc(-c4cccc(-c5cc(-c6ccccc6-c6ccc7c8c(cccc68)-c6ccccc6-7)ccc5C#N)c4)cc3)n2)cc1 ; N#Cc1ccc(-c2ccc(-c3nc(-c4ccccc4)nc(-c4cccc(-c5ccc(-c6cc(-c7cccc(-c8cc9c%10c(cccc%10c8)-c8ccccc8-9)c7)ccc6C#N)cc5)c4)n3)cc2)cc1 ; N#Cc1ccc(-c2cccc(-c3nc(-c4ccccc4)nc(-c4cccc(-c5ccc(-c6cc(-c7ccc(-c8ccc9cccc%10c9c8-c8ccccc8-%10)cc7)ccc6C#N)cc5)c4)n3)c2)cc1 ; N#Cc1ccc(-c2cccc(-c3cccc4c3-c3cccc5cccc-4c35)c2)cc1-c1cccc(-c2ccc(-c3nc(-c4ccccc4)nc(-c4ccc(-c5ccc6ccccc6c5)cc4)n3)cc2)c1]

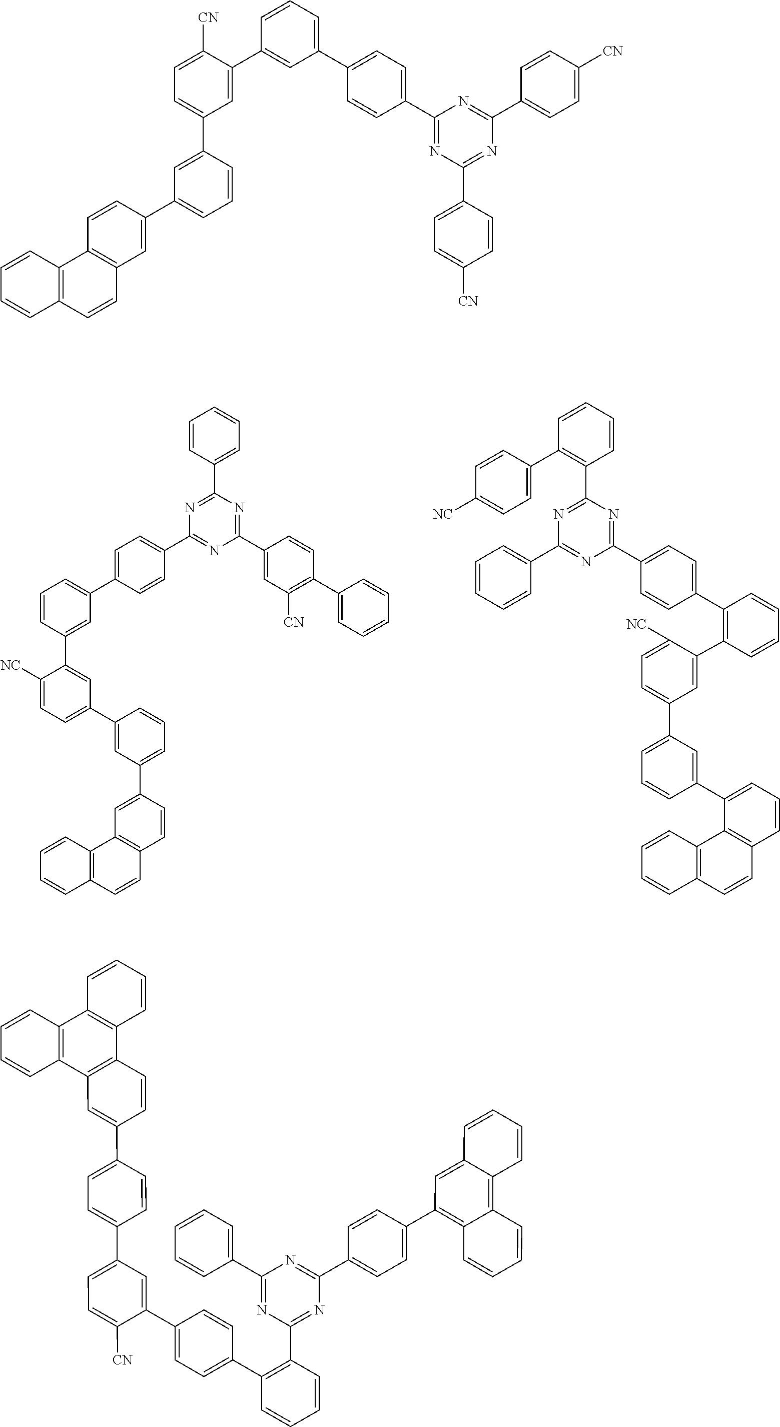 [SMILES: N#Cc1ccc(-c2nc(-c3ccc(C#N)cc3)nc(-c3ccc(-c4cccc(-c5cc(-c6cccc(-c7ccc8c(ccc9ccccc98)c7)c6)ccc5C#N)c4)cc3)n2)cc1 ; N#Cc1cc(-c2nc(-c3ccccc3)nc(-c3ccc(-c4cccc(-c5cc(-c6cccc(-c7ccc8ccc9ccccc9c8c7)c6)ccc5C#N)c4)cc3)n2)ccc1-c1ccccc1 ; N#Cc1ccc(-c2ccccc2-c2nc(-c3ccccc3)nc(-c3ccc(-c4ccccc4-c4cc(-c5cccc(-c6cccc7ccc8ccccc8c67)c5)ccc4C#N)cc3)n2)cc1 ; N#Cc1ccc(-c2ccc(-c3ccc4c5ccccc5c5ccccc5c4c3)cc2)cc1-c1ccc(-c2ccccc2-c2nc(-c3ccccc3)nc(-c3ccc(-c4cc5ccccc5c5ccccc45)cc3)n2)cc1]

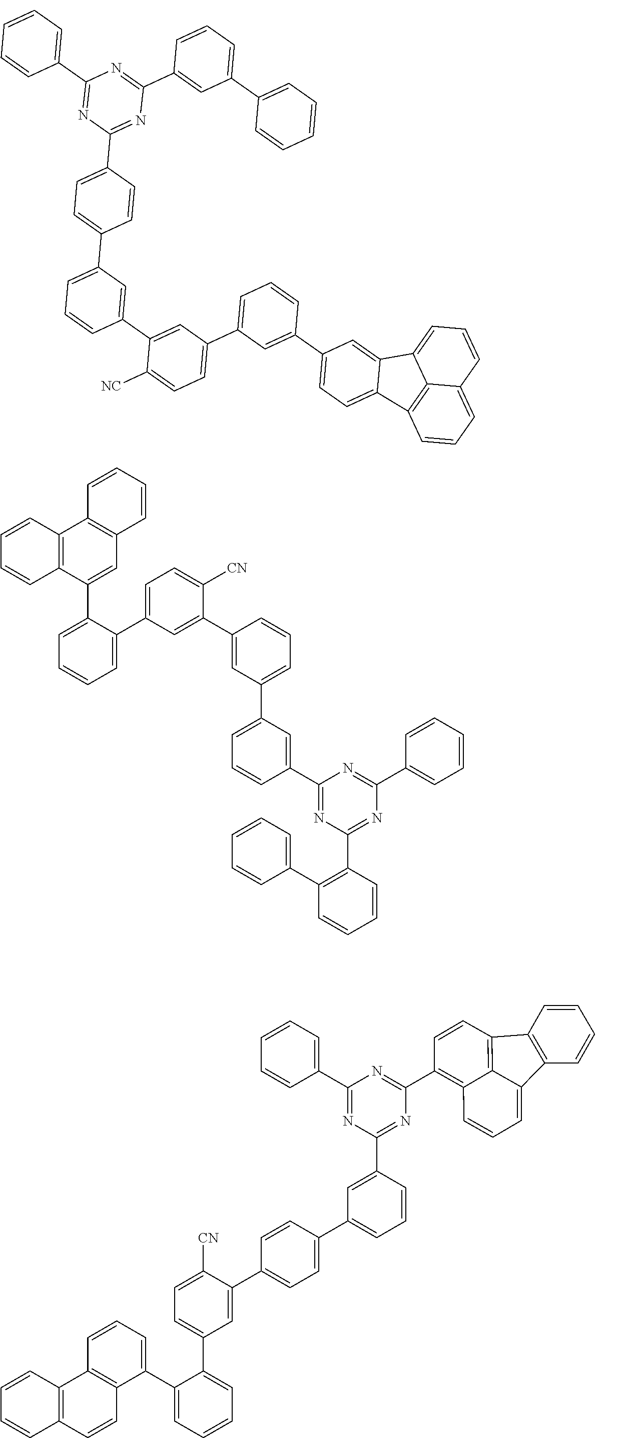 [SMILES: N#Cc1ccc(-c2cccc(-c3ccc4c(c3)-c3cccc5cccc-4c35)c2)cc1-c1cccc(-c2ccc(-c3nc(-c4ccccc4)nc(-c4cccc(-c5ccccc5)c4)n3)cc2)c1 ; N#Cc1ccc(-c2ccccc2-c2cc3ccccc3c3ccccc23)cc1-c1cccc(-c2cccc(-c3nc(-c4ccccc4)nc(-c4ccccc4-c4ccccc4)n3)c2)c1 ; N#Cc1ccc(-c2ccccc2-c2cccc3c2ccc2ccccc23)cc1-c1ccc(-c2cccc(-c3nc(-c4ccccc4)nc(-c4ccc5c6c(cccc46)-c4ccccc4-5)n3)c2)cc1]

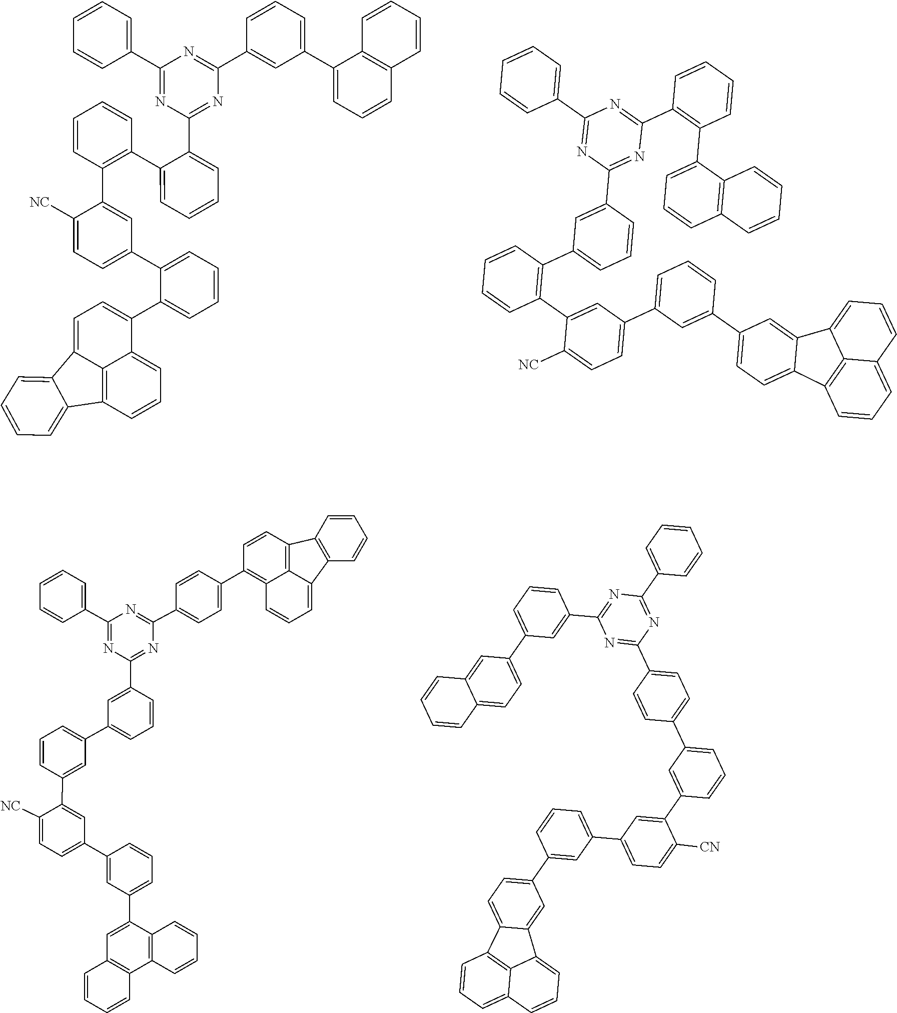 [SMILES: N#Cc1ccc(-c2ccccc2-c2ccc3c4c(cccc24)-c2ccccc2-3)cc1-c1ccccc1-c1ccccc1-c1nc(-c2ccccc2)nc(-c2cccc(-c3cccc4ccccc34)c2)n1 ; N#Cc1ccc(-c2cccc(-c3ccc4c(c3)-c3cccc5cccc-4c35)c2)cc1-c1ccccc1-c1cccc(-c2nc(-c3ccccc3)nc(-c3ccccc3-c3cccc4ccccc34)n2)c1 ; N#Cc1ccc(-c2cccc(-c3cc4ccccc4c4ccccc34)c2)cc1-c1cccc(-c2cccc(-c3nc(-c4ccccc4)nc(-c4ccc(-c5ccc6c7c(cccc57)-c5ccccc5-6)cc4)n3)c2)c1 ; N#Cc1ccc(-c2cccc(-c3ccc4c(c3)-c3cccc5cccc-4c35)c2)cc1-c1cccc(-c2ccc(-c3nc(-c4ccccc4)nc(-c4cccc(-c5ccc6ccccc6c5)c4)n3)cc2)c1]

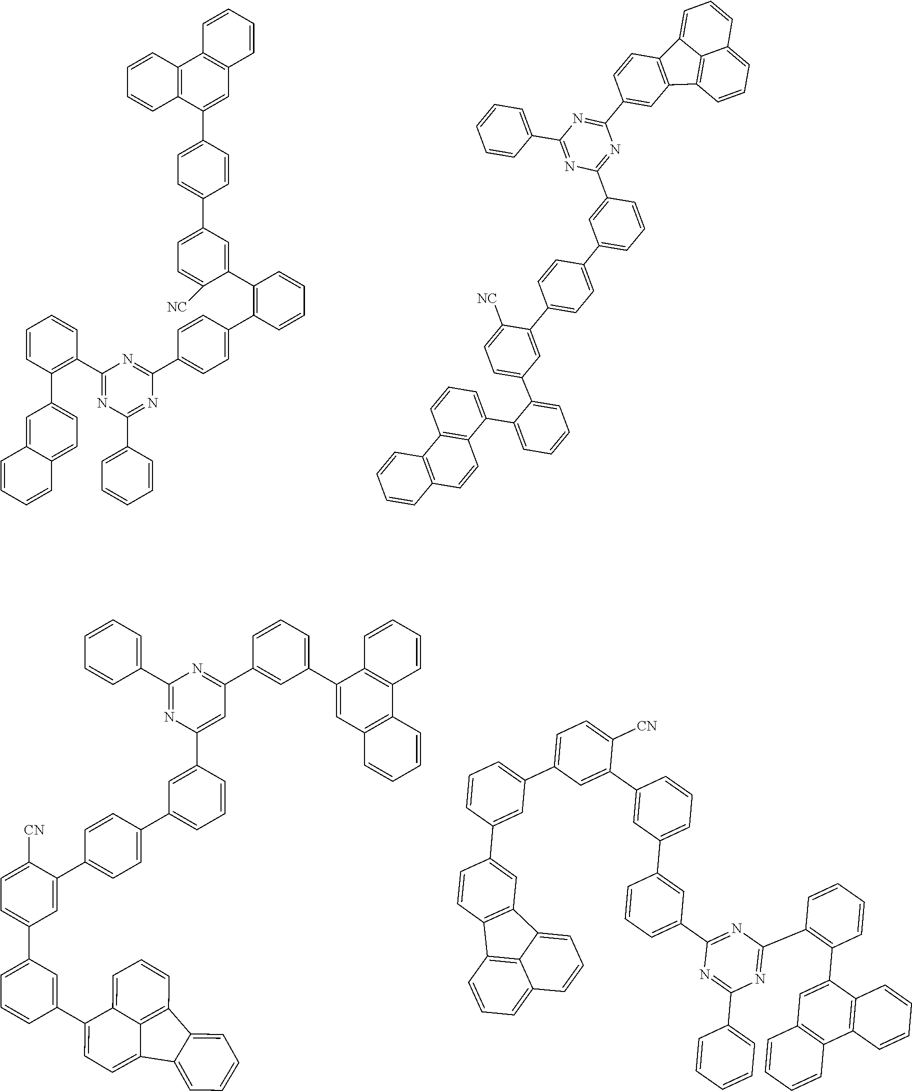 [SMILES: N#Cc1ccc(-c2ccc(-c3cc4ccccc4c4ccccc34)cc2)cc1-c1ccccc1-c1ccc(-c2nc(-c3ccccc3)nc(-c3ccccc3-c3ccc4ccccc4c3)n2)cc1 ; N#Cc1ccc(-c2ccccc2-c2cccc3c2ccc2ccccc23)cc1-c1ccc(-c2cccc(-c3nc(-c4ccccc4)nc(-c4ccc5c(c4)-c4cccc6cccc-5c46)n3)c2)cc1 ; N#Cc1ccc(-c2cccc(-c3ccc4c5c(cccc35)-c3ccccc3-4)c2)cc1-c1ccc(-c2cccc(-c3cc(-c4cccc(-c5cc6ccccc6c6ccccc56)c4)nc(-c4ccccc4)n3)c2)cc1 ; N#Cc1ccc(-c2cccc(-c3ccc4c(c3)-c3cccc5cccc-4c35)c2)cc1-c1cccc(-c2cccc(-c3nc(-c4ccccc4)nc(-c4ccccc4-c4cc5ccccc5c5ccccc45)n3)c2)c1]

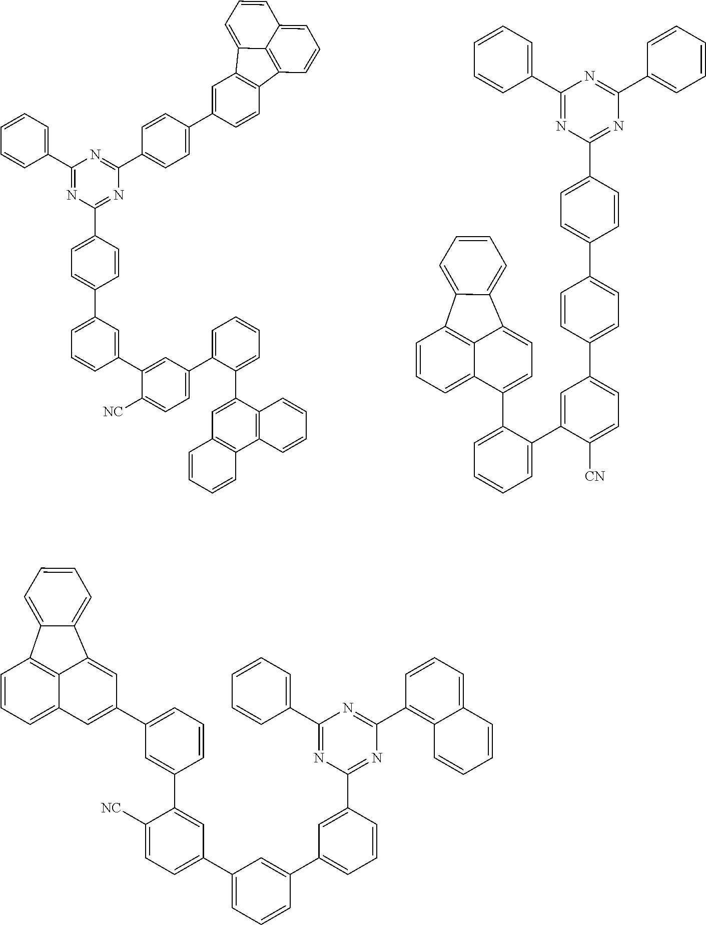 [SMILES: N#Cc1ccc(-c2ccccc2-c2cc3ccccc3c3ccccc23)cc1-c1cccc(-c2ccc(-c3nc(-c4ccccc4)nc(-c4ccc(-c5ccc6c(c5)-c5cccc7cccc-6c57)cc4)n3)cc2)c1 ; N#Cc1ccc(-c2ccc(-c3ccc(-c4nc(-c5ccccc5)nc(-c5ccccc5)n4)cc3)cc2)cc1-c1ccccc1-c1ccc2c3c(cccc13)-c1ccccc1-2 ; N#Cc1ccc(-c2cccc(-c3cccc(-c4nc(-c5ccccc5)nc(-c5cccc6ccccc56)n4)c3)c2)cc1-c1cccc(-c2cc3c4c(cccc4c2)-c2ccccc2-3)c1]

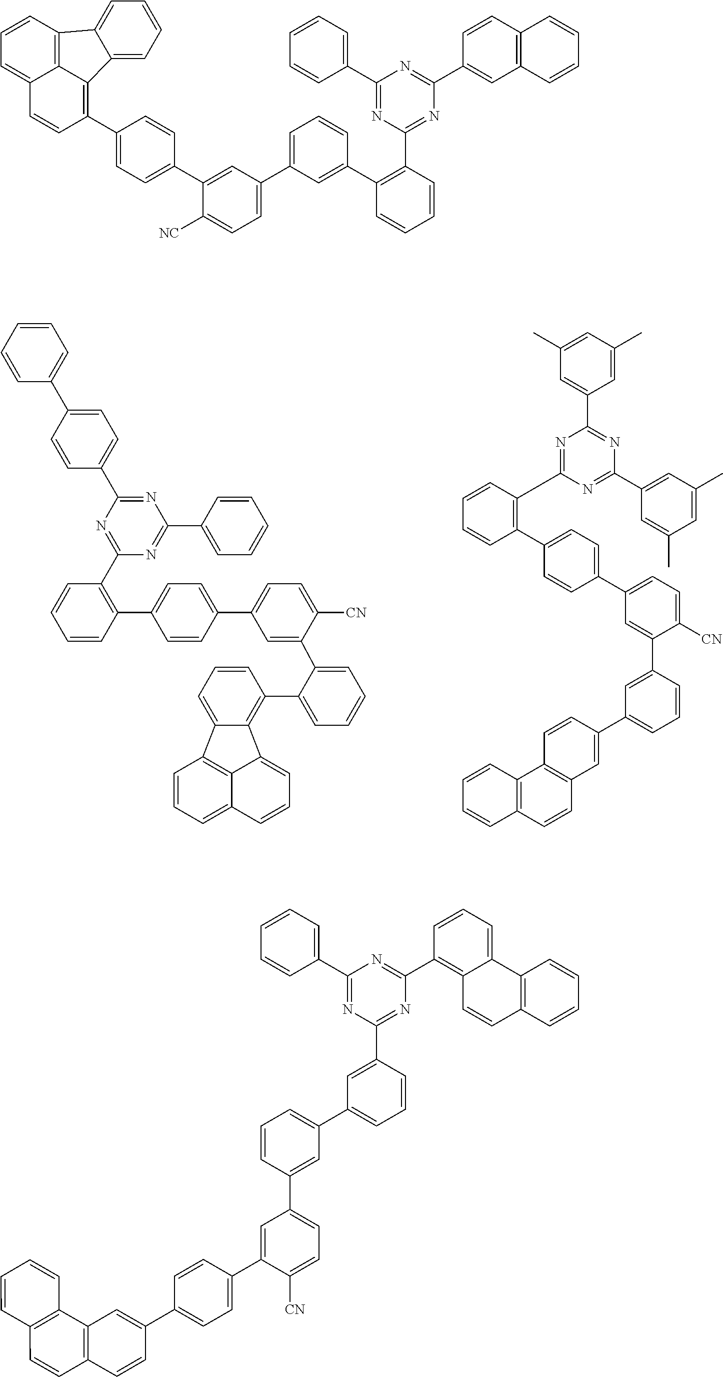 [SMILES: N#Cc1ccc(-c2cccc(-c3ccccc3-c3nc(-c4ccccc4)nc(-c4ccc5ccccc5c4)n3)c2)cc1-c1ccc(-c2ccc3cccc4c3c2-c2ccccc2-4)cc1 ; N#Cc1ccc(-c2ccc(-c3ccccc3-c3nc(-c4ccccc4)nc(-c4ccc(-c5ccccc5)cc4)n3)cc2)cc1-c1ccccc1-c1cccc2c1-c1cccc3cccc-2c13 ; Cc1cc(C)cc(-c2nc(-c3cc(C)cc(C)c3)nc(-c3ccccc3-c3ccc(-c4ccc(C#N)c(-c5cccc(-c6ccc7c(ccc8ccccc87)c6)c5)c4)cc3)n2)c1 ; N#Cc1ccc(-c2cccc(-c3cccc(-c4nc(-c5ccccc5)nc(-c5cccc6c5ccc5ccccc56)n4)c3)c2)cc1-c1ccc(-c2ccc3ccc4ccccc4c3c2)cc1]

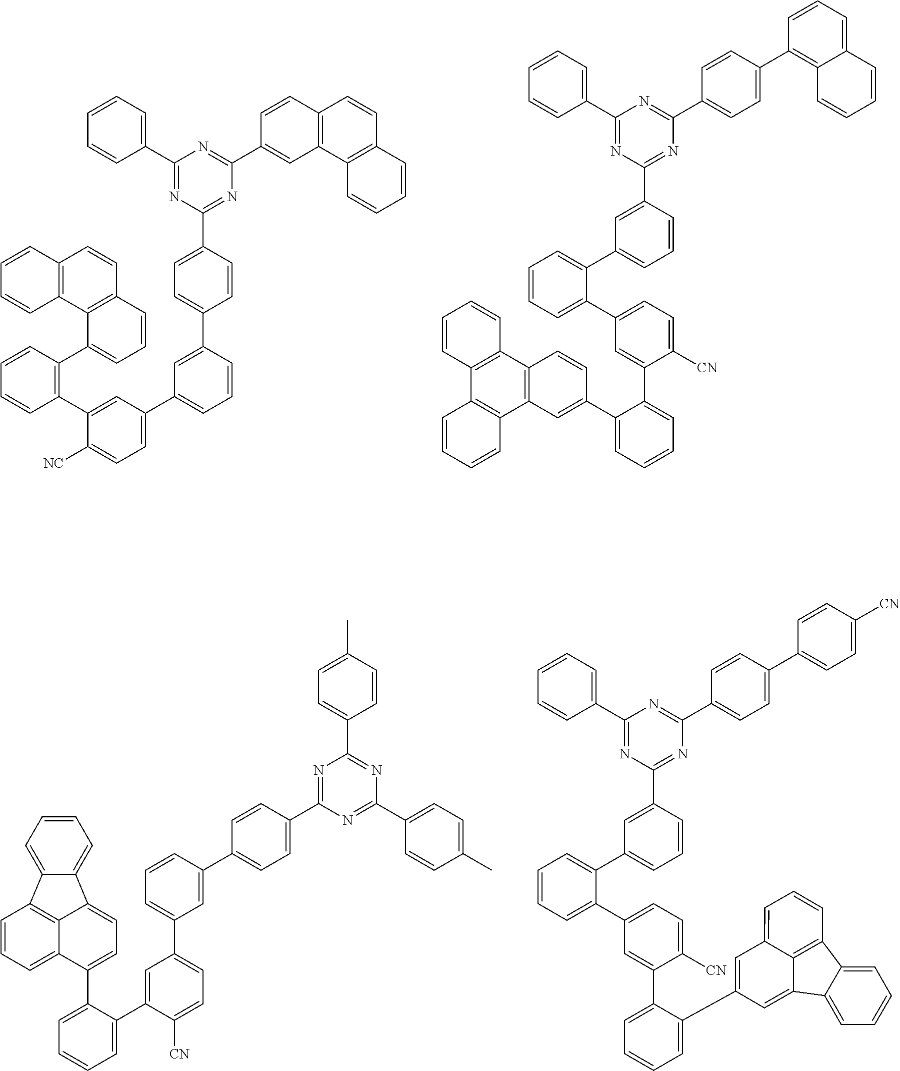

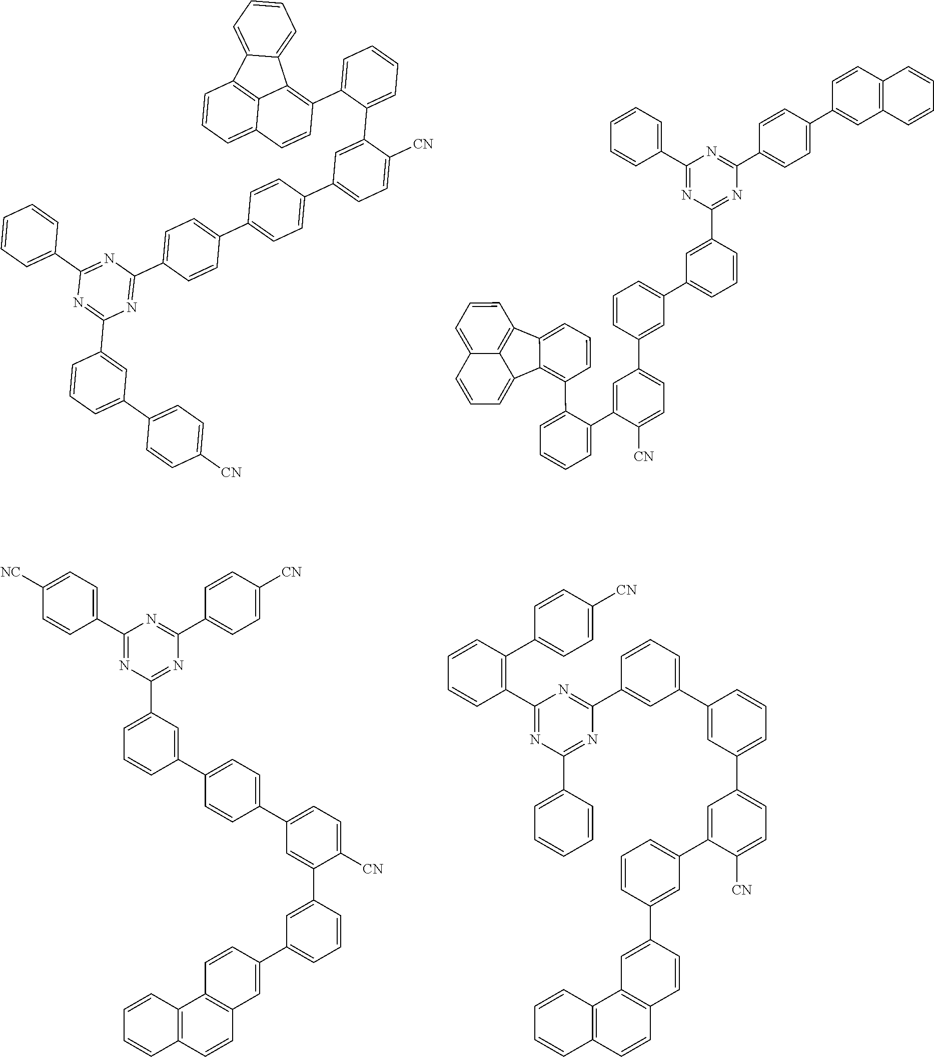 [SMILES: N#Cc1ccc(-c2cccc(-c3nc(-c4ccccc4)nc(-c4ccc(-c5ccc(-c6ccc(C#N)c(-c7ccccc7-c7ccc8cccc9c8c7-c7ccccc7-9)c6)cc5)cc4)n3)c2)cc1 ; N#Cc1ccc(-c2cccc(-c3cccc(-c4nc(-c5ccccc5)nc(-c5ccc(-c6ccc7ccccc7c6)cc5)n4)c3)c2)cc1-c1ccccc1-c1cccc2c1-c1cccc3cccc-2c13 ; N#Cc1ccc(-c2nc(-c3ccc(C#N)cc3)nc(-c3cccc(-c4ccc(-c5ccc(C#N)c(-c6cccc(-c7ccc8c(ccc9ccccc98)c7)c6)c5)cc4)c3)n2)cc1 ; N#Cc1ccc(-c2ccccc2-c2nc(-c3ccccc3)nc(-c3cccc(-c4cccc(-c5ccc(C#N)c(-c6cccc(-c7ccc8ccc9ccccc9c8c7)c6)c5)c4)c3)n2)cc1]

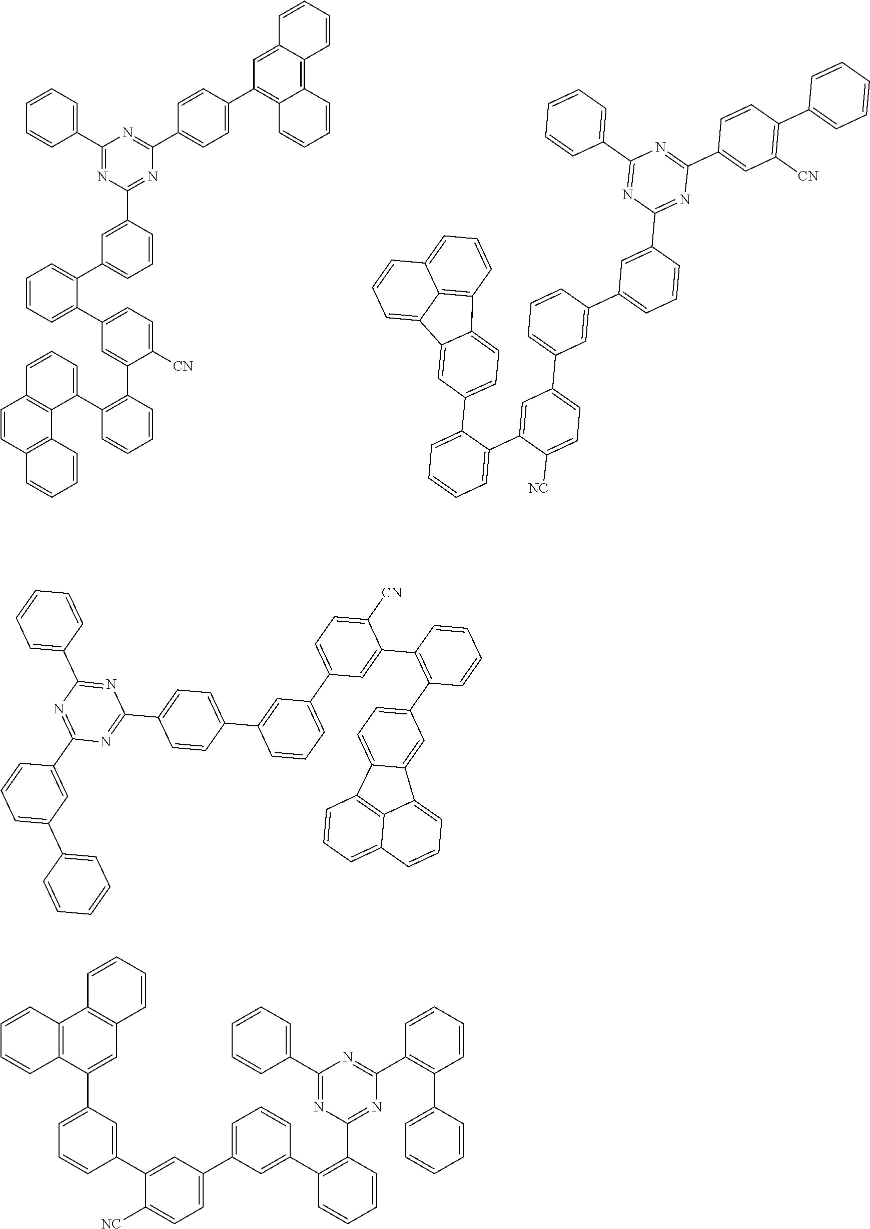

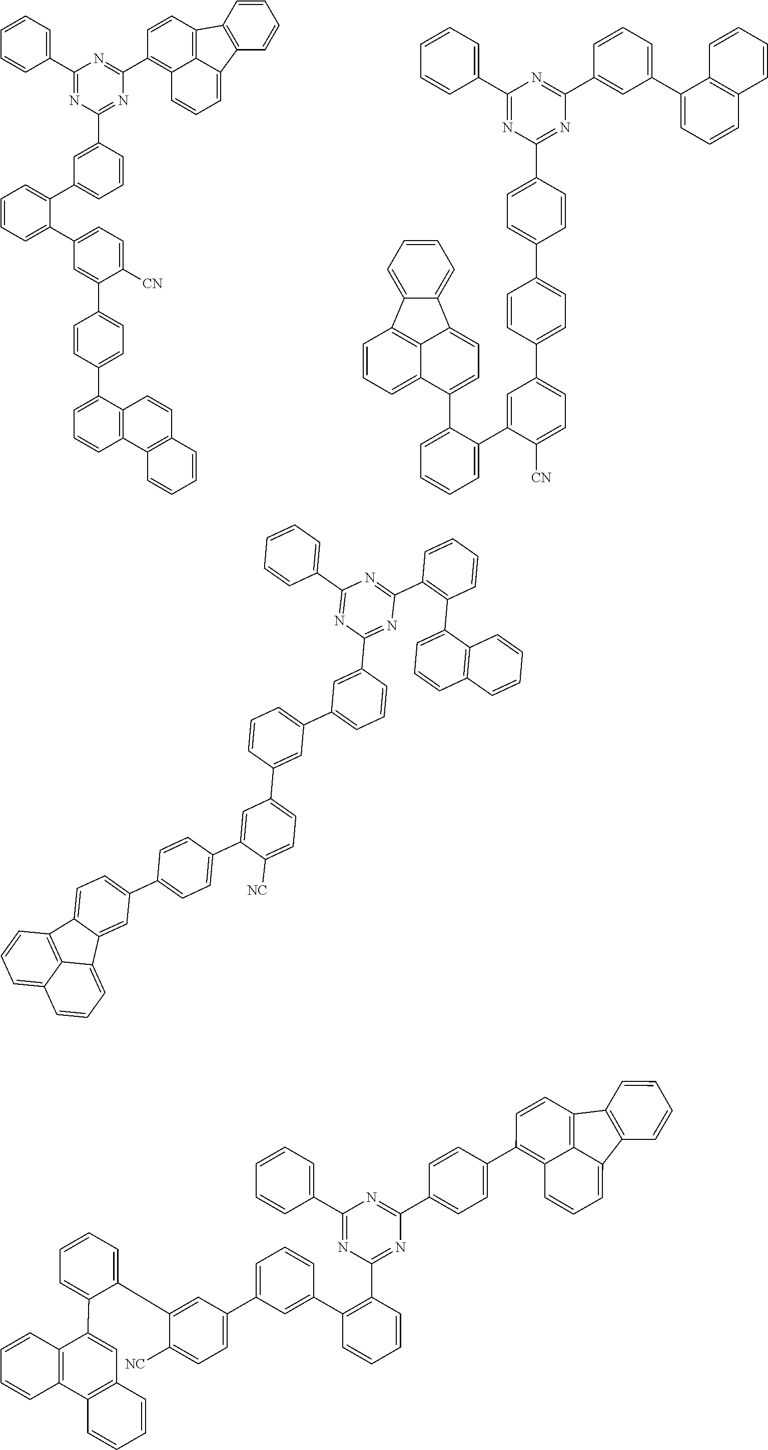 [SMILES: N#Cc1ccc(-c2ccccc2-c2cccc(-c3nc(-c4ccccc4)nc(-c4ccc5c6c(cccc46)-c4ccccc4-5)n3)c2)cc1-c1ccc(-c2cccc3c2ccc2ccccc23)cc1 ; N#Cc1ccc(-c2ccc(-c3ccc(-c4nc(-c5ccccc5)nc(-c5cccc(-c6cccc7ccccc67)c5)n4)cc3)cc2)cc1-c1ccccc1-c1ccc2c3c(cccc13)-c1ccccc1-2 ; N#Cc1ccc(-c2cccc(-c3cccc(-c4nc(-c5ccccc5)nc(-c5ccccc5-c5cccc6ccccc56)n4)c3)c2)cc1-c1ccc(-c2ccc3c(c2)-c2cccc4cccc-3c24)cc1 ; N#Cc1ccc(-c2cccc(-c3ccccc3-c3nc(-c4ccccc4)nc(-c4ccc(-c5ccc6c7c(cccc57)-c5ccccc5-6)cc4)n3)c2)cc1-c1ccccc1-c1cc2ccccc2c2ccccc12]

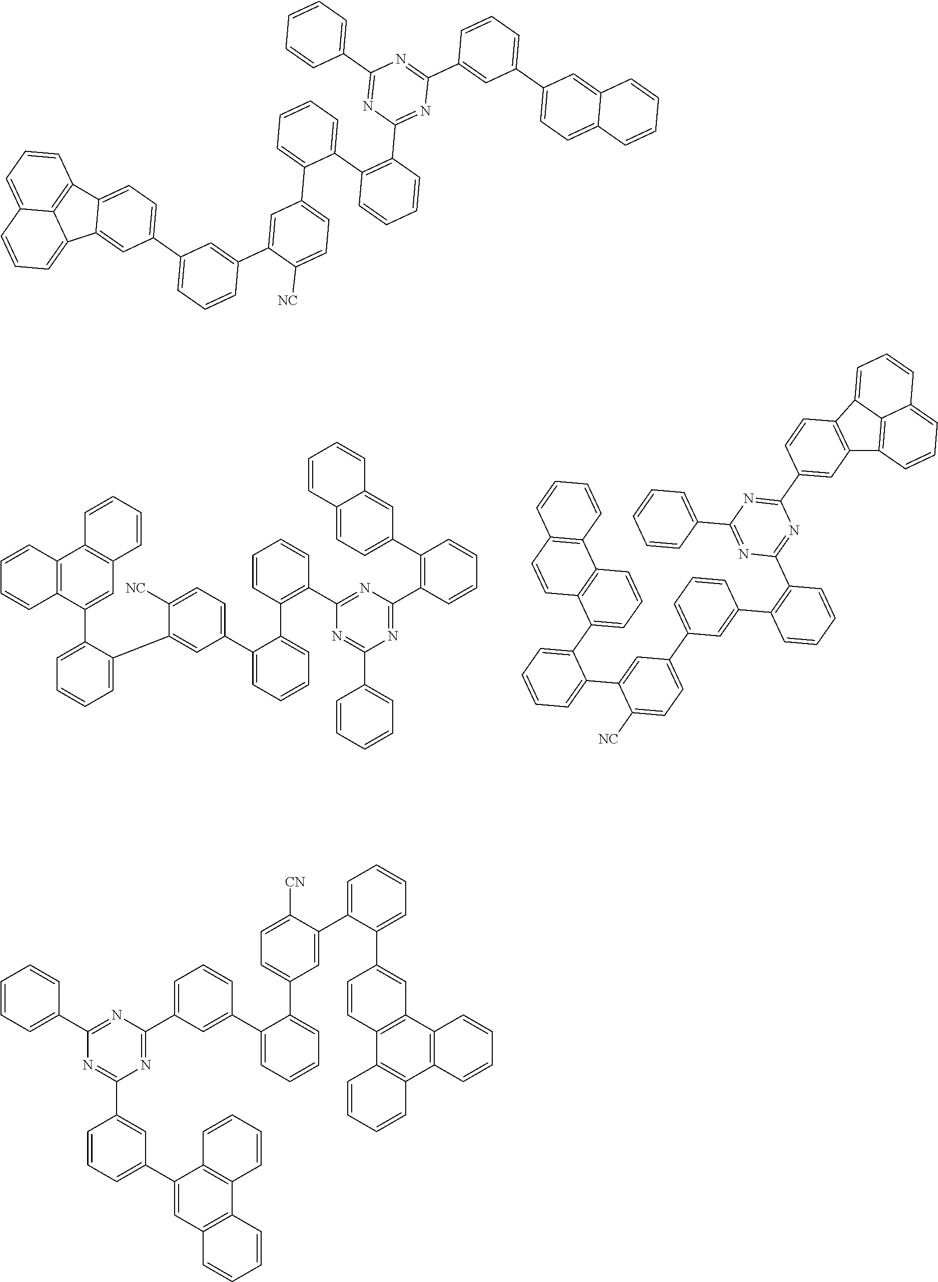 [SMILES: N#Cc1ccc(-c2ccccc2-c2ccccc2-c2nc(-c3ccccc3)nc(-c3cccc(-c4ccc5ccccc5c4)c3)n2)cc1-c1cccc(-c2ccc3c(c2)-c2cccc4cccc-3c24)c1 ; N#Cc1ccc(-c2ccccc2-c2ccccc2-c2nc(-c3ccccc3)nc(-c3ccccc3-c3ccc4ccccc4c3)n2)cc1-c1ccccc1-c1cc2ccccc2c2ccccc12 ; N#Cc1ccc(-c2cccc(-c3ccccc3-c3nc(-c4ccccc4)nc(-c4ccc5c(c4)-c4cccc6cccc-5c46)n3)c2)cc1-c1ccccc1-c1cccc2c1ccc1ccccc12 ; N#Cc1ccc(-c2ccccc2-c2cccc(-c3nc(-c4ccccc4)nc(-c4cccc(-c5cc6ccccc6c6ccccc56)c4)n3)c2)cc1-c1ccccc1-c1ccc2c3ccccc3c3ccccc3c2c1]

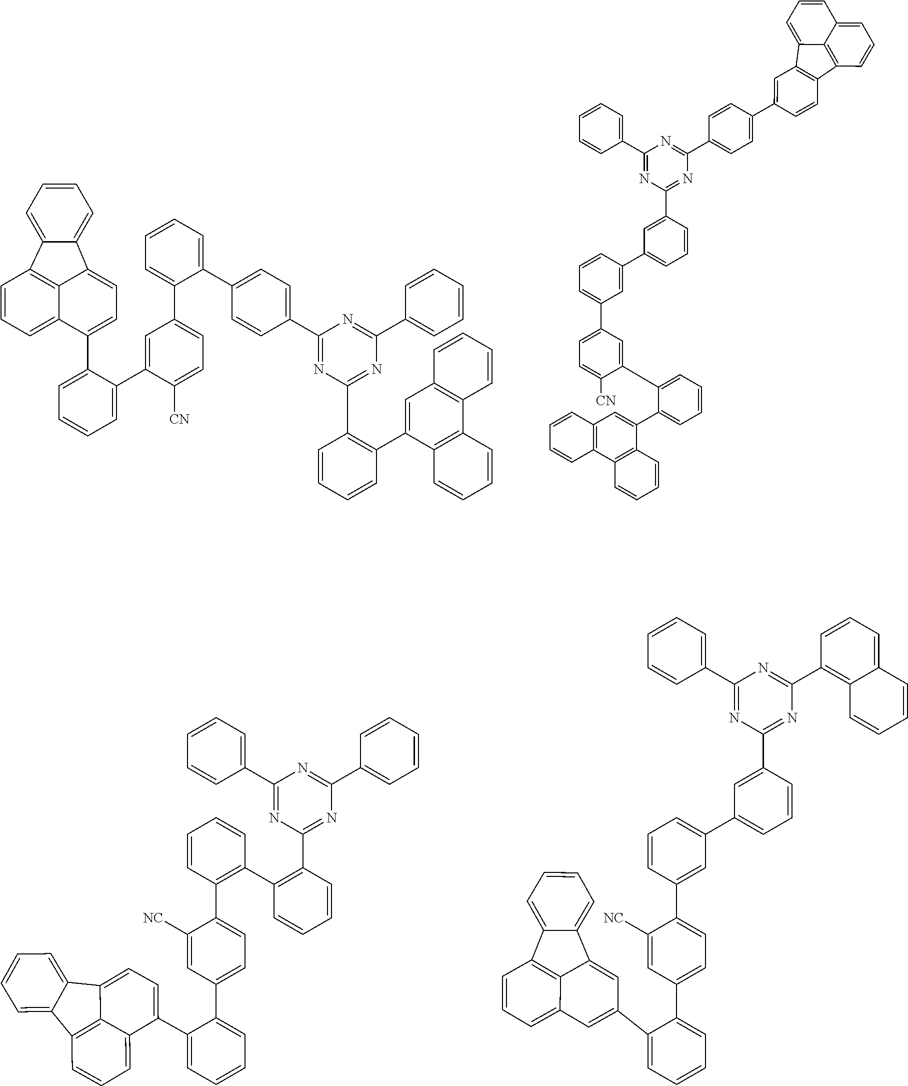 [SMILES: N#Cc1ccc(-c2ccccc2-c2ccc(-c3nc(-c4ccccc4)nc(-c4ccccc4-c4cc5ccccc5c5ccccc45)n3)cc2)cc1-c1ccccc1-c1ccc2c3c(cccc13)-c1ccccc1-2 ; N#Cc1ccc(-c2cccc(-c3cccc(-c4nc(-c5ccccc5)nc(-c5ccc(-c6ccc7c(c6)-c6cccc8cccc-7c68)cc5)n4)c3)c2)cc1-c1ccccc1-c1cc2ccccc2c2ccccc12 ; N#Cc1cc(-c2ccccc2-c2ccc3c4c(cccc24)-c2ccccc2-3)ccc1-c1ccccc1-c1ccccc1-c1nc(-c2ccccc2)nc(-c2ccccc2)n1 ; N#Cc1cc(-c2ccccc2-c2cc3c4c(cccc4c2)-c2ccccc2-3)ccc1-c1cccc(-c2cccc(-c3nc(-c4ccccc4)nc(-c4cccc5ccccc45)n3)c2)c1]

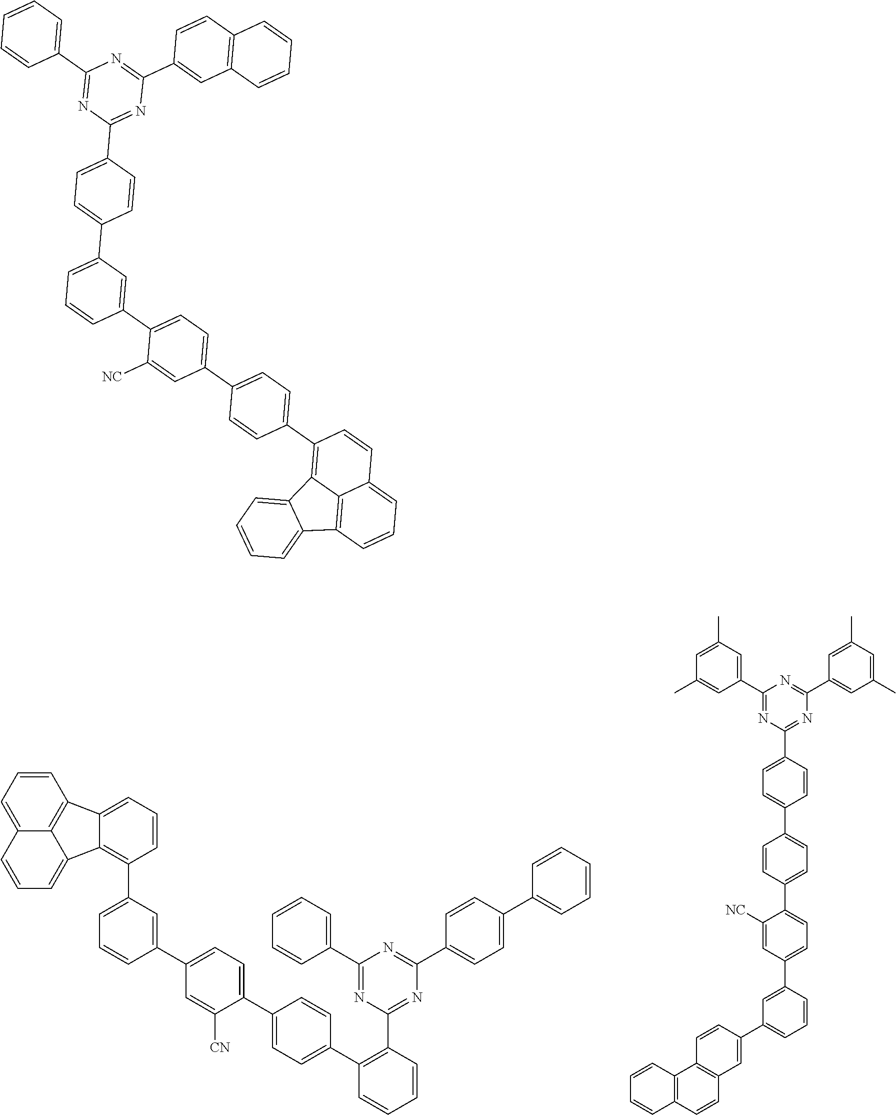 [SMILES: N#Cc1cc(-c2ccc(-c3ccc4cccc5c4c3-c3ccccc3-5)cc2)ccc1-c1cccc(-c2ccc(-c3nc(-c4ccccc4)nc(-c4ccc5ccccc5c4)n3)cc2)c1 ; N#Cc1cc(-c2cccc(-c3cccc4c3-c3cccc5cccc-4c35)c2)ccc1-c1ccc(-c2ccccc2-c2nc(-c3ccccc3)nc(-c3ccc(-c4ccccc4)cc3)n2)cc1 ; Cc1cc(C)cc(-c2nc(-c3ccc(-c4ccc(-c5ccc(-c6cccc(-c7ccc8c(ccc9ccccc98)c7)c6)cc5C#N)cc4)cc3)nc(-c3cc(C)cc(C)c3)n2)c1]

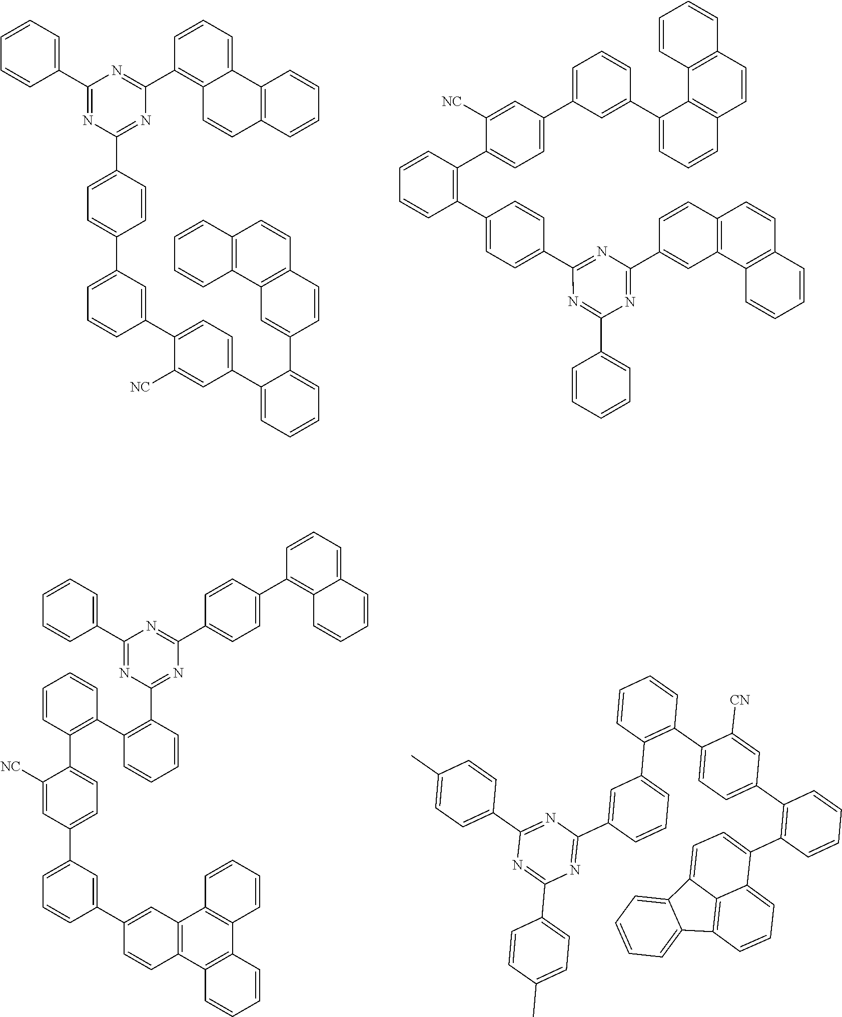 [SMILES: N#Cc1cc(-c2ccccc2-c2ccc3ccc4ccccc4c3c2)ccc1-c1cccc(-c2ccc(-c3nc(-c4ccccc4)nc(-c4cccc5c4ccc4ccccc45)n3)cc2)c1 ; N#Cc1cc(-c2cccc(-c3cccc4ccc5ccccc5c34)c2)ccc1-c1ccccc1-c1ccc(-c2nc(-c3ccccc3)nc(-c3ccc4ccc5ccccc5c4c3)n2)cc1 ; N#Cc1cc(-c2cccc(-c3ccc4c5ccccc5c5ccccc5c4c3)c2)ccc1-c1ccccc1-c1ccccc1-c1nc(-c2ccccc2)nc(-c2ccc(-c3cccc4ccccc34)cc2)n1 ; Cc1ccc(-c2nc(-c3ccc(C)cc3)nc(-c3cccc(-c4ccccc4-c4ccc(-c5ccccc5-c5ccc6c7c(cccc57)-c5ccccc5-6)cc4C#N)c3)n2)cc1]

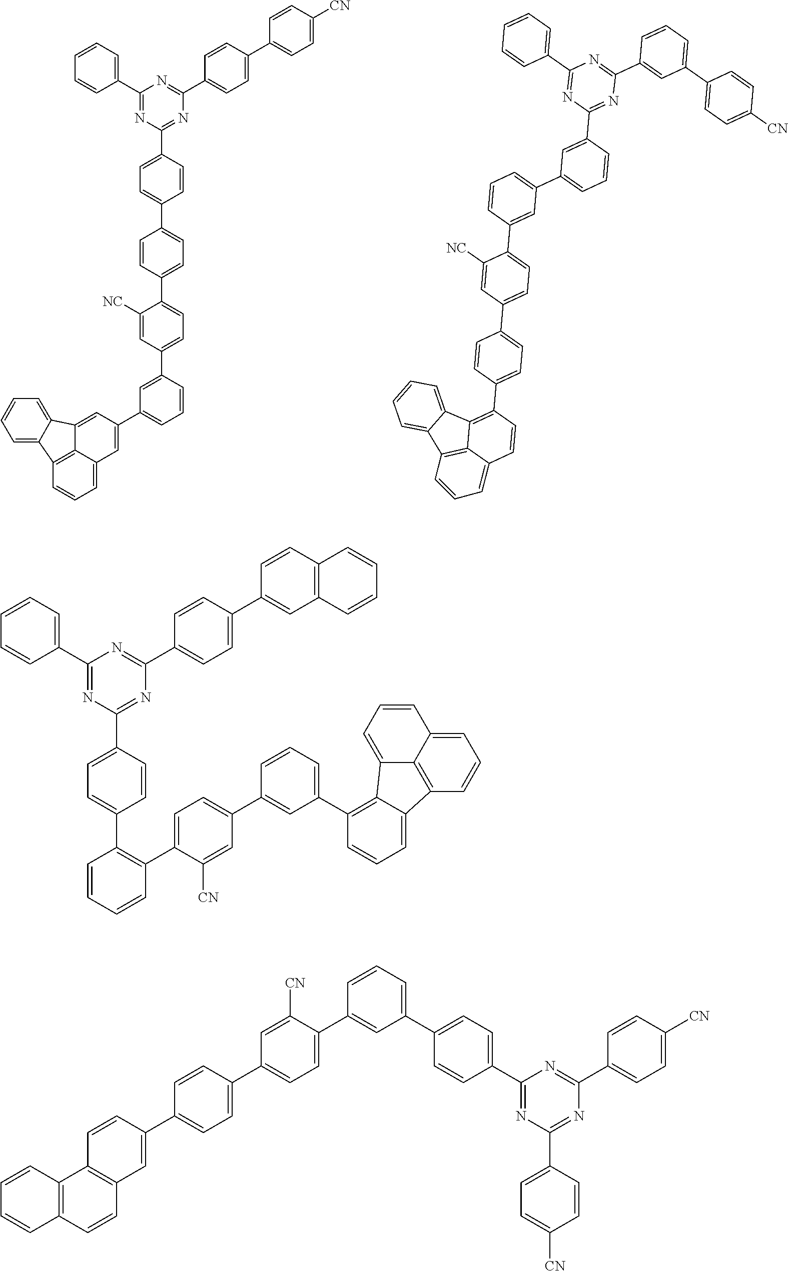 [SMILES: N#Cc1ccc(-c2ccc(-c3nc(-c4ccccc4)nc(-c4ccc(-c5ccc(-c6ccc(-c7cccc(-c8cc9c%10c(cccc%10c8)-c8ccccc8-9)c7)cc6C#N)cc5)cc4)n3)cc2)cc1 ; N#Cc1ccc(-c2cccc(-c3nc(-c4ccccc4)nc(-c4cccc(-c5cccc(-c6ccc(-c7ccc(-c8ccc9cccc%10c9c8-c8ccccc8-%10)cc7)cc6C#N)c5)c4)n3)c2)cc1 ; N#Cc1cc(-c2cccc(-c3cccc4c3-c3cccc5cccc-4c35)c2)ccc1-c1ccccc1-c1ccc(-c2nc(-c3ccccc3)nc(-c3ccc(-c4ccc5ccccc5c4)cc3)n2)cc1 ; N#Cc1ccc(-c2nc(-c3ccc(C#N)cc3)nc(-c3ccc(-c4cccc(-c5ccc(-c6ccc(-c7ccc8c(ccc9ccccc98)c7)cc6)cc5C#N)c4)cc3)n2)cc1]

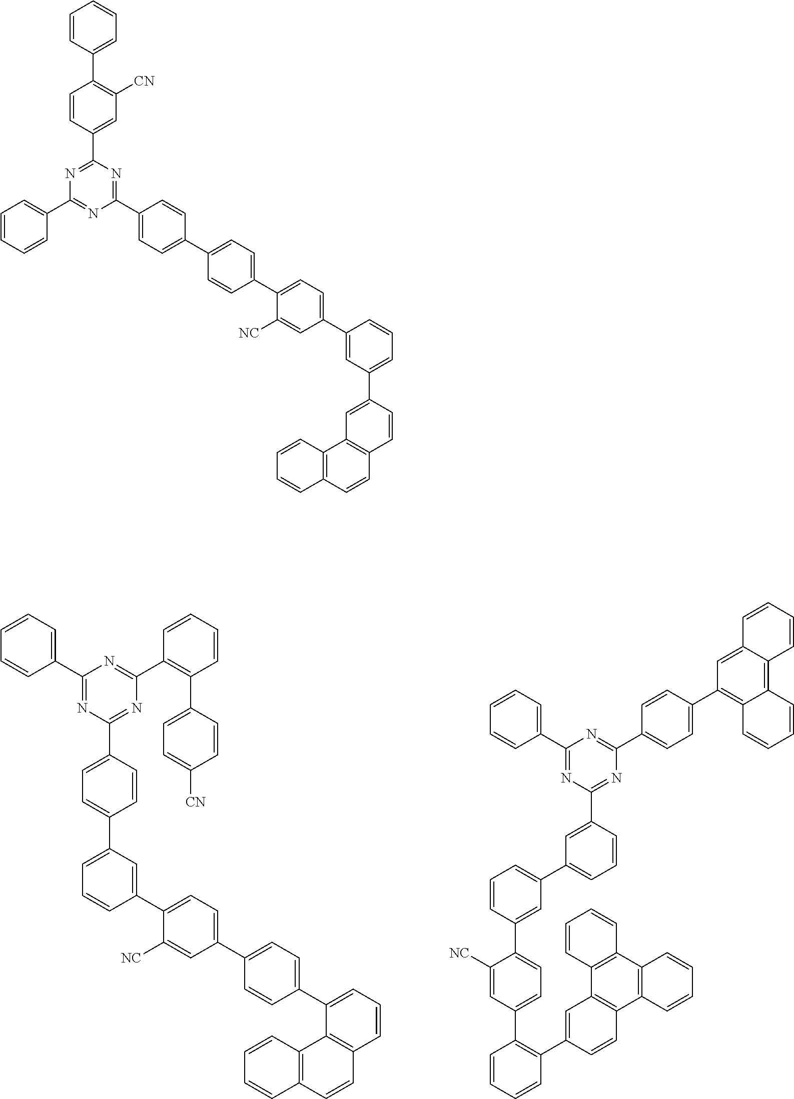 [SMILES: N#Cc1cc(-c2cccc(-c3ccc4ccc5ccccc5c4c3)c2)ccc1-c1ccc(-c2ccc(-c3nc(-c4ccccc4)nc(-c4ccc(-c5ccccc5)c(C#N)c4)n3)cc2)cc1 ; N#Cc1ccc(-c2ccccc2-c2nc(-c3ccccc3)nc(-c3ccc(-c4cccc(-c5ccc(-c6ccc(-c7cccc8ccc9ccccc9c78)cc6)cc5C#N)c4)cc3)n2)cc1 ; N#Cc1cc(-c2ccccc2-c2ccc3c4ccccc4c4ccccc4c3c2)ccc1-c1cccc(-c2cccc(-c3nc(-c4ccccc4)nc(-c4ccc(-c5cc6ccccc6c6ccccc56)cc4)n3)c2)c1]

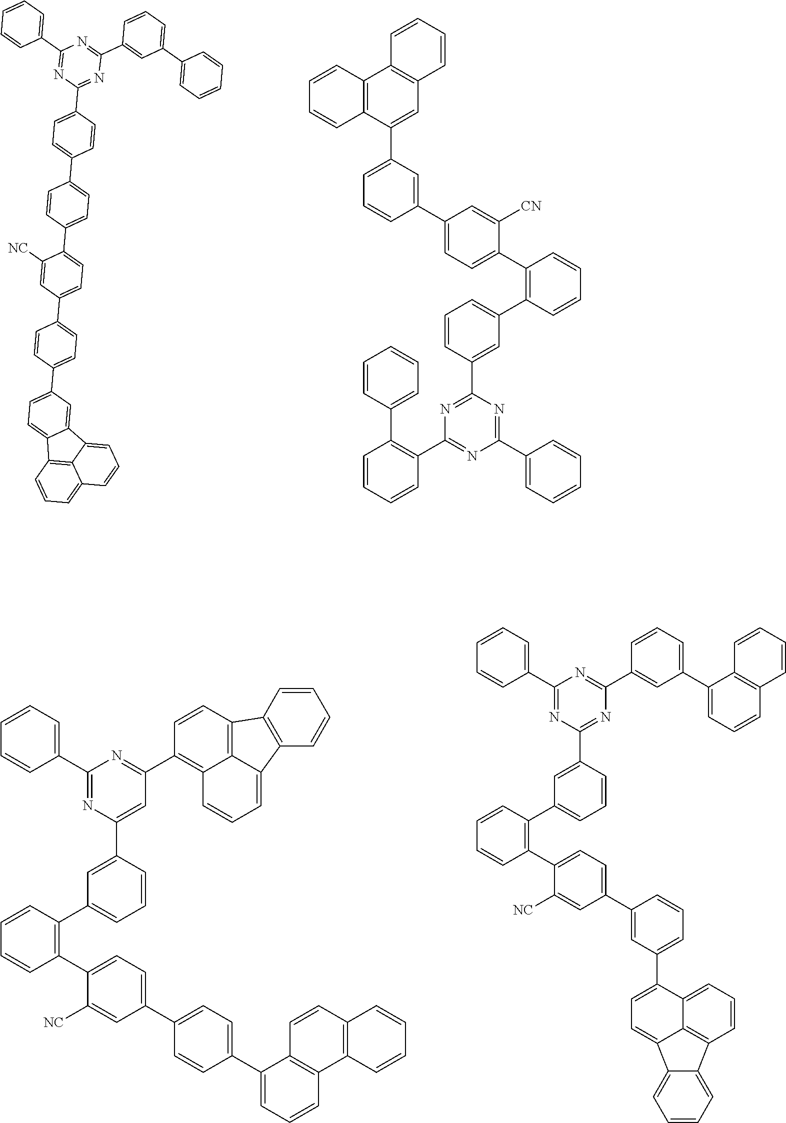 [SMILES: N#Cc1cc(-c2ccc(-c3ccc4c(c3)-c3cccc5cccc-4c35)cc2)ccc1-c1ccc(-c2ccc(-c3nc(-c4ccccc4)nc(-c4cccc(-c5ccccc5)c4)n3)cc2)cc1 ; N#Cc1cc(-c2cccc(-c3cc4ccccc4c4ccccc34)c2)ccc1-c1ccccc1-c1cccc(-c2nc(-c3ccccc3)nc(-c3ccccc3-c3ccccc3)n2)c1 ; N#Cc1cc(-c2ccc(-c3cccc4c3ccc3ccccc34)cc2)ccc1-c1ccccc1-c1cccc(-c2cc(-c3ccc4c5c(cccc35)-c3ccccc3-4)nc(-c3ccccc3)n2)c1 ; N#Cc1cc(-c2cccc(-c3ccc4c5c(cccc35)-c3ccccc3-4)c2)ccc1-c1ccccc1-c1cccc(-c2nc(-c3ccccc3)nc(-c3cccc(-c4cccc5ccccc45)c3)n2)c1]

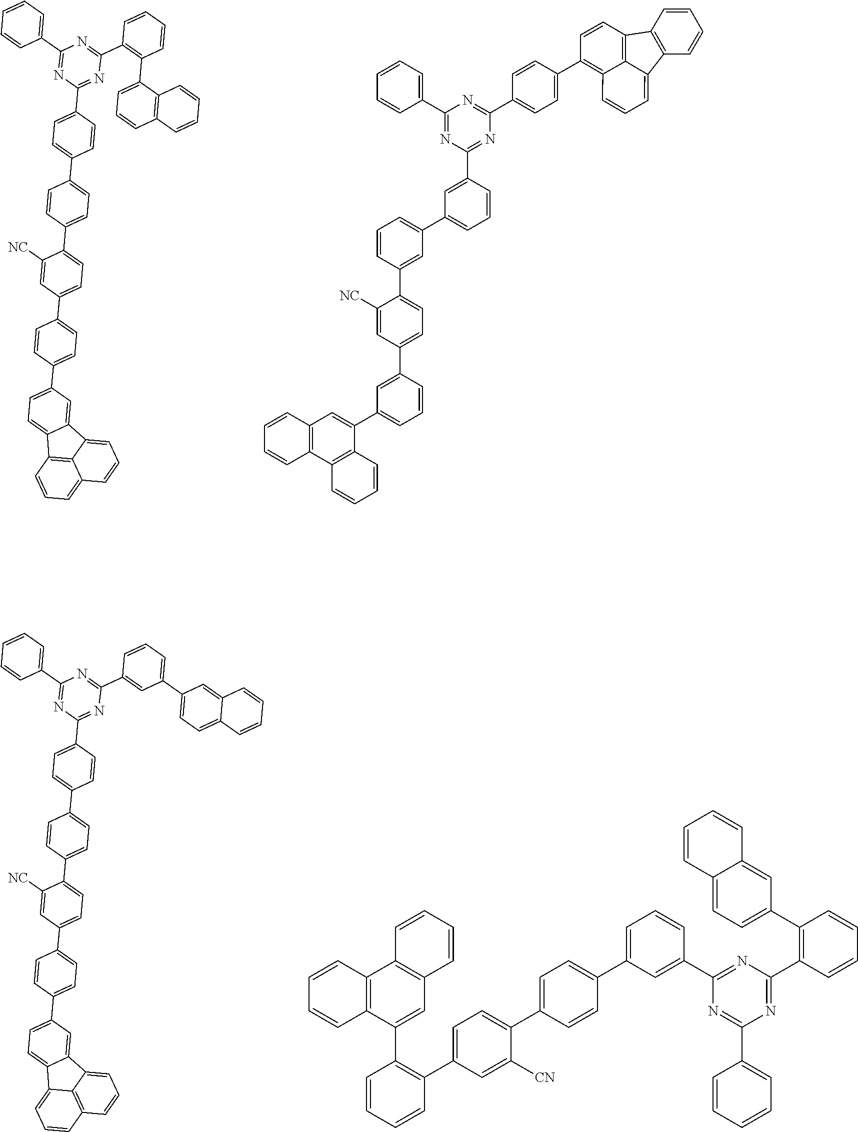 [SMILES: N#Cc1cc(-c2ccc(-c3ccc4c(c3)-c3cccc5cccc-4c35)cc2)ccc1-c1ccc(-c2ccc(-c3nc(-c4ccccc4)nc(-c4ccccc4-c4cccc5ccccc45)n3)cc2)cc1 ; N#Cc1cc(-c2cccc(-c3cc4ccccc4c4ccccc34)c2)ccc1-c1cccc(-c2cccc(-c3nc(-c4ccccc4)nc(-c4ccc(-c5ccc6c7c(cccc57)-c5ccccc5-6)cc4)n3)c2)c1 ; N#Cc1cc(-c2ccc(-c3ccc4c(c3)-c3cccc5cccc-4c35)cc2)ccc1-c1ccc(-c2ccc(-c3nc(-c4ccccc4)nc(-c4cccc(-c5ccc6ccccc6c5)c4)n3)cc2)cc1 ; N#Cc1cc(-c2ccccc2-c2cc3ccccc3c3ccccc23)ccc1-c1ccc(-c2cccc(-c3nc(-c4ccccc4)nc(-c4ccccc4-c4ccc5ccccc5c4)n3)c2)cc1]

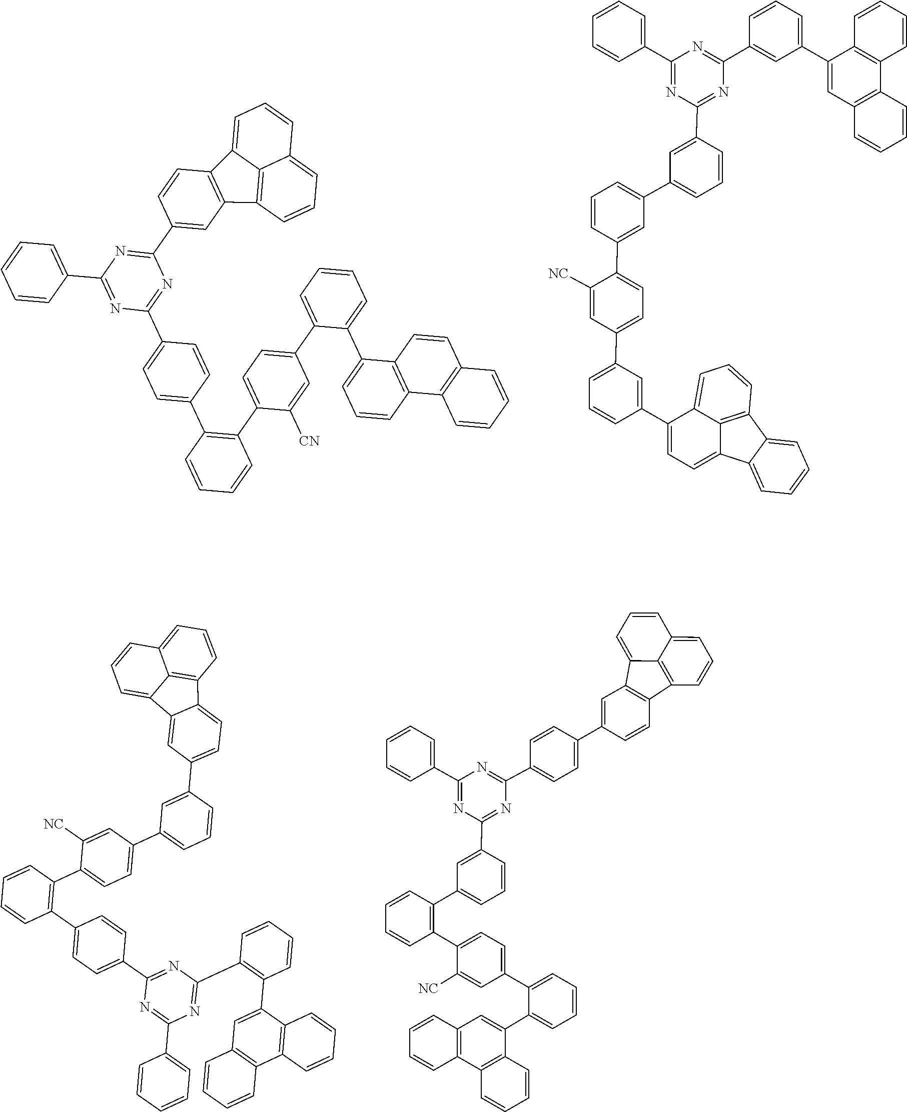

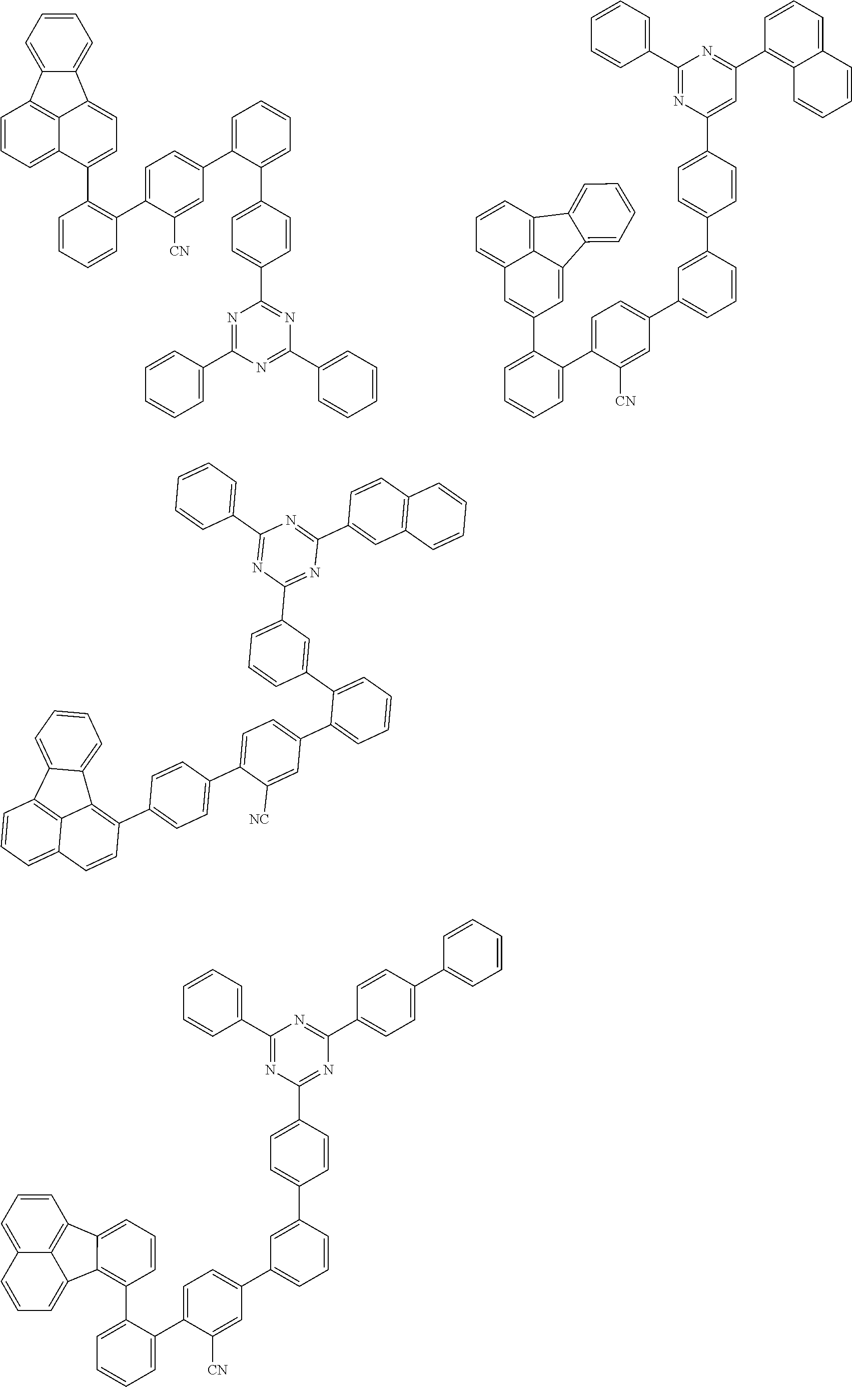 [SMILES: N#Cc1cc(-c2ccccc2-c2ccc(-c3nc(-c4ccccc4)nc(-c4ccccc4)n3)cc2)ccc1-c1ccccc1-c1ccc2c3c(cccc13)-c1ccccc1-2 ; N#Cc1cc(-c2cccc(-c3ccc(-c4cc(-c5cccc6ccccc56)nc(-c5ccccc5)n4)cc3)c2)ccc1-c1ccccc1-c1cc2c3c(cccc3c1)-c1ccccc1-2 ; N#Cc1cc(-c2ccccc2-c2cccc(-c3nc(-c4ccccc4)nc(-c4ccc5ccccc5c4)n3)c2)ccc1-c1ccc(-c2ccc3cccc4c3c2-c2ccccc2-4)cc1 ; N#Cc1cc(-c2cccc(-c3ccc(-c4nc(-c5ccccc5)nc(-c5ccc(-c6ccccc6)cc5)n4)cc3)c2)ccc1-c1ccccc1-c1cccc2c1-c1cccc3cccc-2c13]

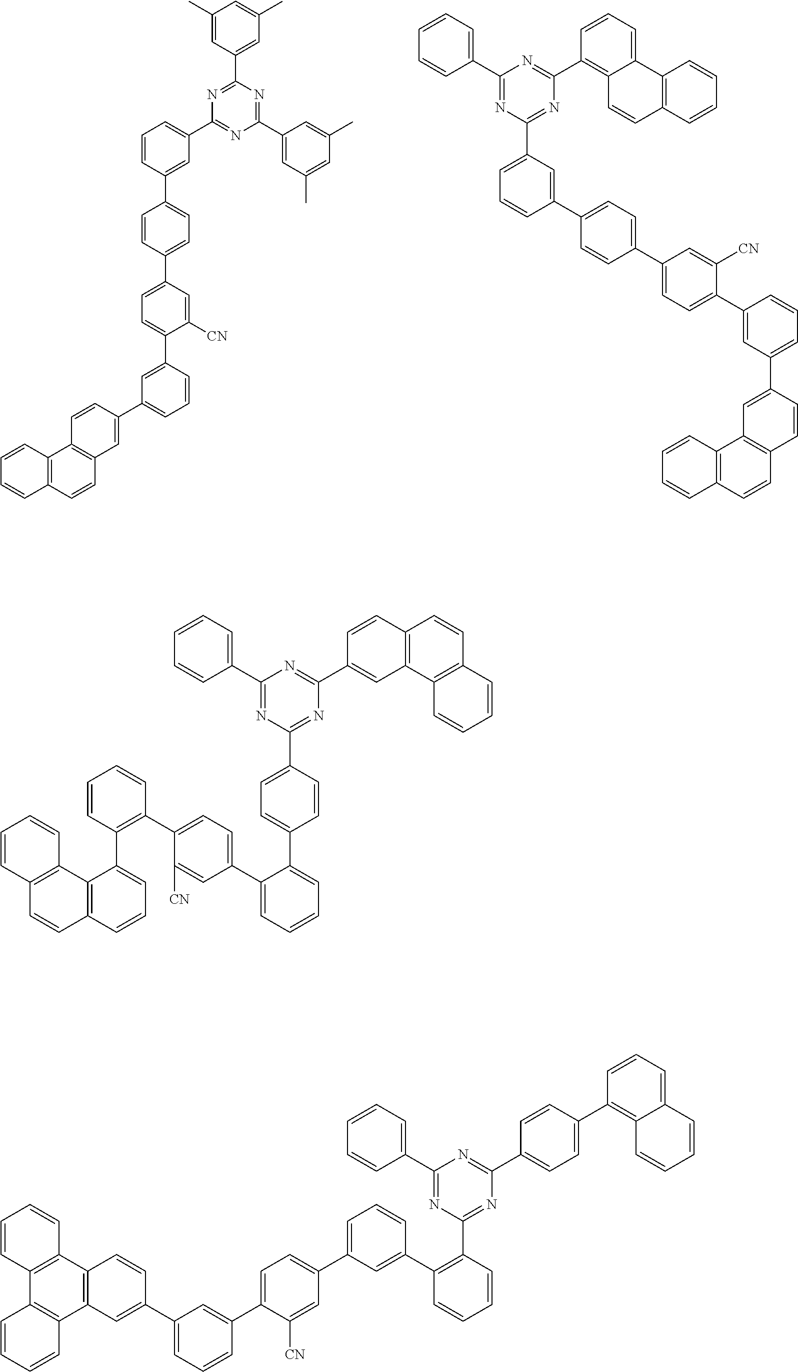

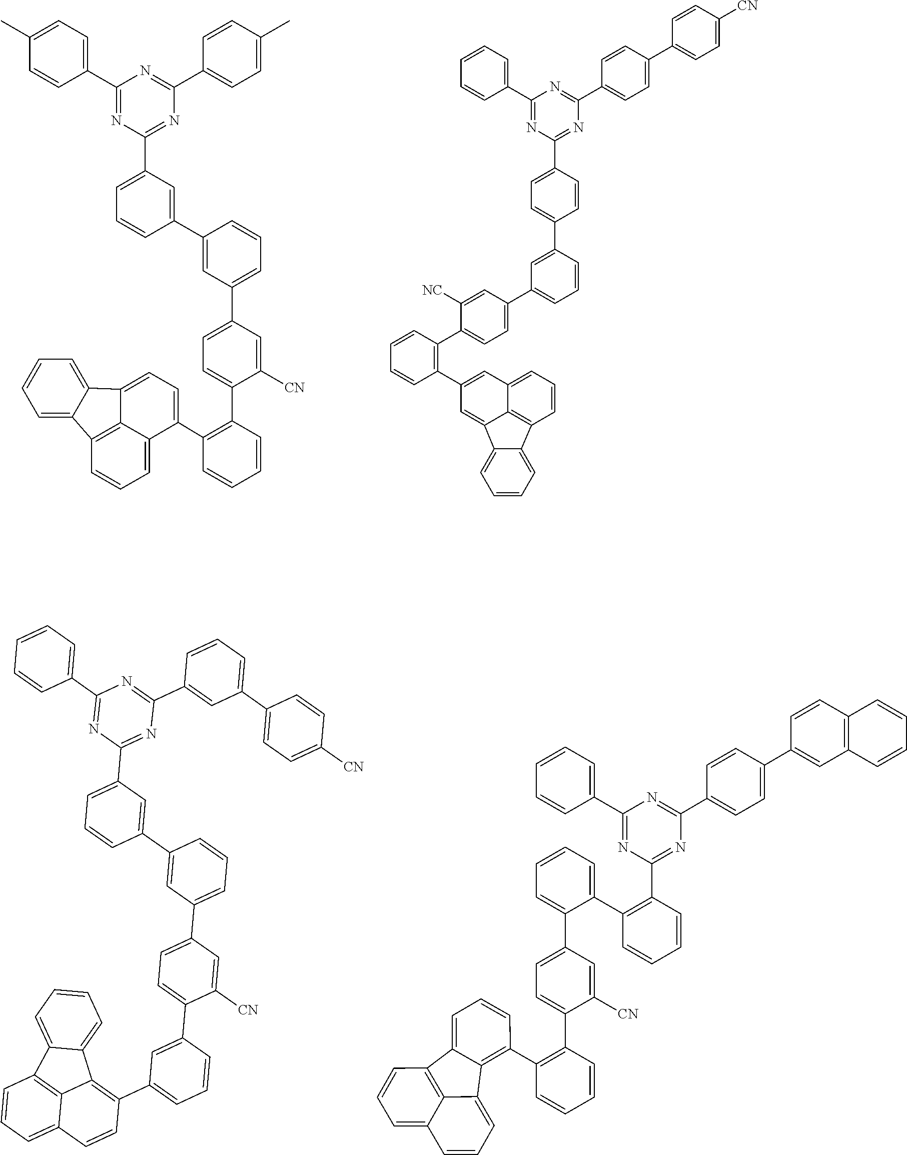 [SMILES: Cc1ccc(-c2nc(-c3ccc(C)cc3)nc(-c3cccc(-c4cccc(-c5ccc(-c6ccccc6-c6ccc7c8c(cccc68)-c6ccccc6-7)c(C#N)c5)c4)c3)n2)cc1 ; N#Cc1ccc(-c2ccc(-c3nc(-c4ccccc4)nc(-c4ccc(-c5cccc(-c6ccc(-c7ccccc7-c7cc8c9c(cccc9c7)-c7ccccc7-8)c(C#N)c6)c5)cc4)n3)cc2)cc1 ; N#Cc1ccc(-c2cccc(-c3nc(-c4ccccc4)nc(-c4cccc(-c5cccc(-c6ccc(-c7cccc(-c8ccc9cccc%10c9c8-c8ccccc8-%10)c7)c(C#N)c6)c5)c4)n3)c2)cc1 ; N#Cc1cc(-c2ccccc2-c2ccccc2-c2nc(-c3ccccc3)nc(-c3ccc(-c4ccc5ccccc5c4)cc3)n2)ccc1-c1ccccc1-c1cccc2c1-c1cccc3cccc-2c13]

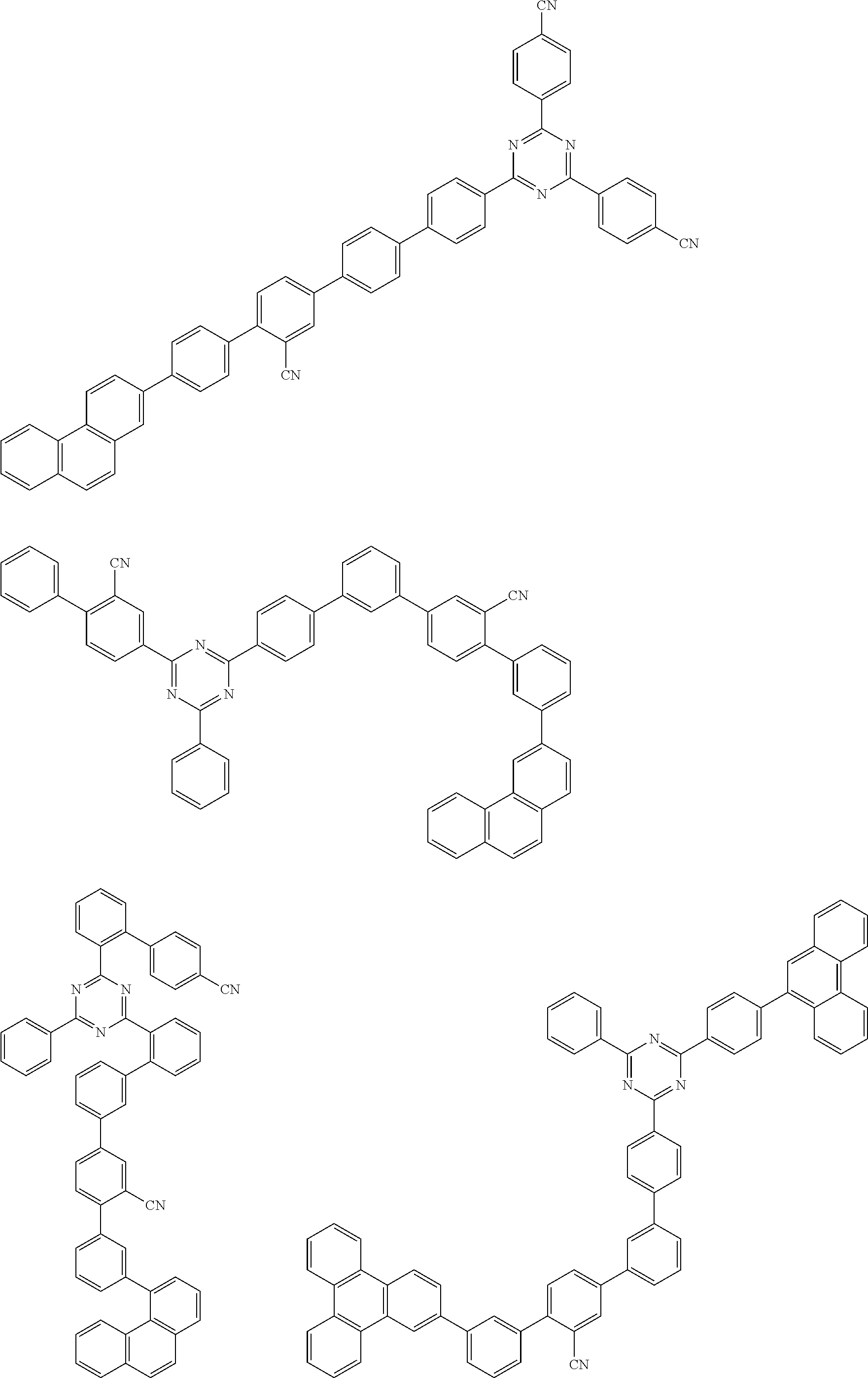 [SMILES: N#Cc1ccc(-c2nc(-c3ccc(C#N)cc3)nc(-c3ccc(-c4ccc(-c5ccc(-c6ccc(-c7ccc8c(ccc9ccccc98)c7)cc6)c(C#N)c5)cc4)cc3)n2)cc1 ; N#Cc1cc(-c2cccc(-c3ccc(-c4nc(-c5ccccc5)nc(-c5ccc(-c6ccccc6)c(C#N)c5)n4)cc3)c2)ccc1-c1cccc(-c2ccc3ccc4ccccc4c3c2)c1 ; N#Cc1ccc(-c2ccccc2-c2nc(-c3ccccc3)nc(-c3ccccc3-c3cccc(-c4ccc(-c5cccc(-c6cccc7ccc8ccccc8c67)c5)c(C#N)c4)c3)n2)cc1 ; N#Cc1cc(-c2cccc(-c3ccc(-c4nc(-c5ccccc5)nc(-c5ccc(-c6cc7ccccc7c7ccccc67)cc5)n4)cc3)c2)ccc1-c1cccc(-c2ccc3c4ccccc4c4ccccc4c3c2)c1]

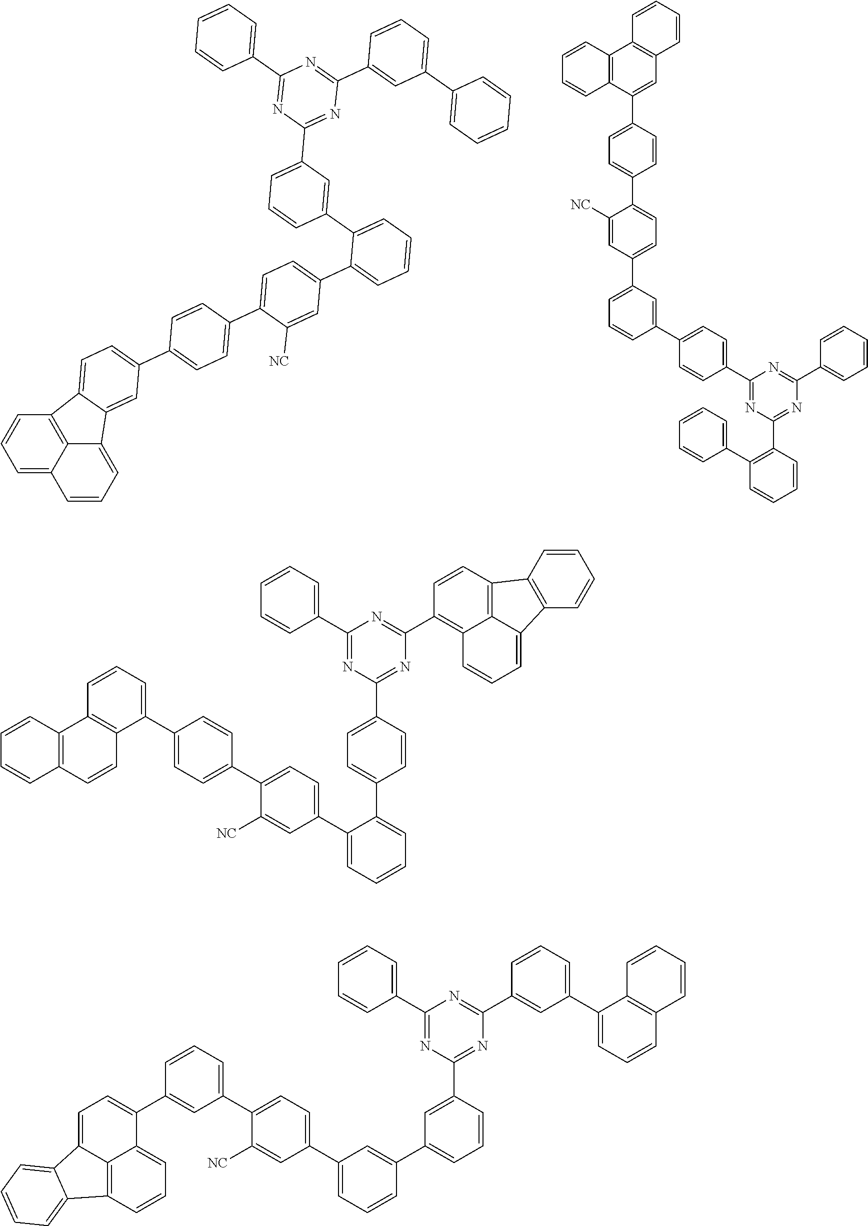 [SMILES: N#Cc1cc(-c2ccccc2-c2cccc(-c3nc(-c4ccccc4)nc(-c4cccc(-c5ccccc5)c4)n3)c2)ccc1-c1ccc(-c2ccc3c(c2)-c2cccc4cccc-3c24)cc1 ; N#Cc1cc(-c2cccc(-c3ccc(-c4nc(-c5ccccc5)nc(-c5ccccc5-c5ccccc5)n4)cc3)c2)ccc1-c1ccc(-c2cc3ccccc3c3ccccc23)cc1 ; N#Cc1cc(-c2ccccc2-c2ccc(-c3nc(-c4ccccc4)nc(-c4ccc5c6c(cccc46)-c4ccccc4-5)n3)cc2)ccc1-c1ccc(-c2cccc3c2ccc2ccccc23)cc1 ; N#Cc1cc(-c2cccc(-c3cccc(-c4nc(-c5ccccc5)nc(-c5cccc(-c6cccc7ccccc67)c5)n4)c3)c2)ccc1-c1cccc(-c2ccc3c4c(cccc24)-c2ccccc2-3)c1]

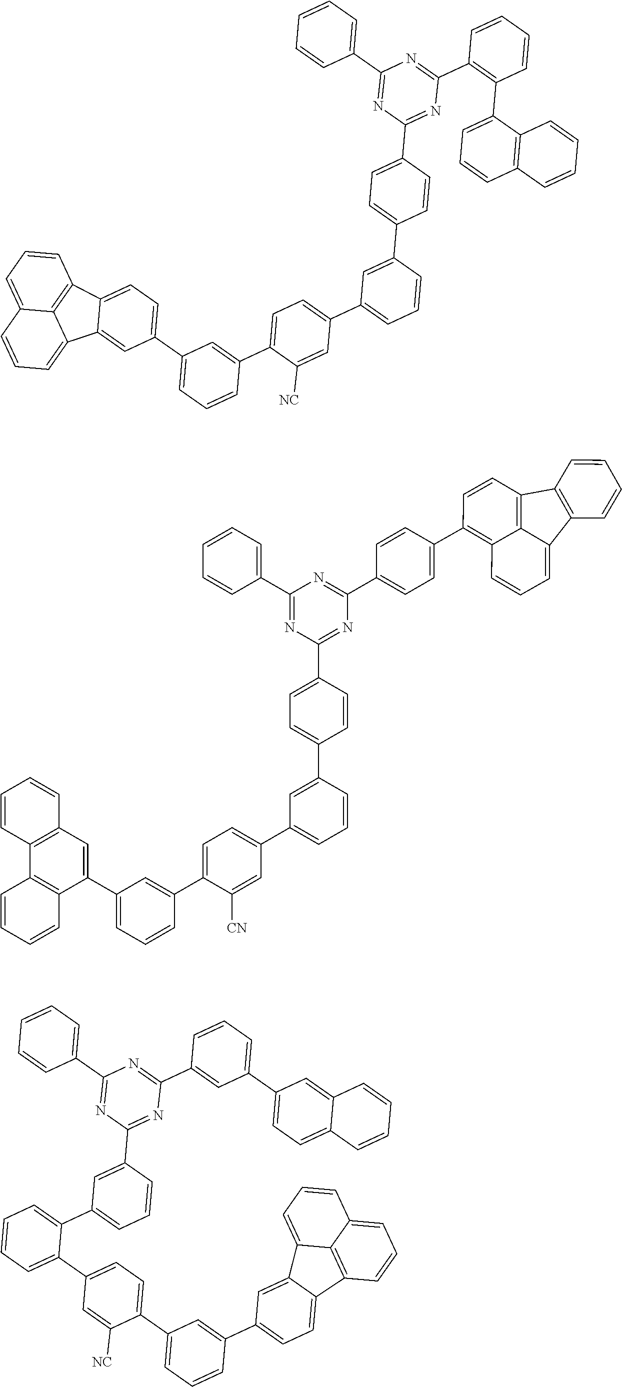 [SMILES: N#Cc1cc(-c2cccc(-c3ccc(-c4nc(-c5ccccc5)nc(-c5ccccc5-c5cccc6ccccc56)n4)cc3)c2)ccc1-c1cccc(-c2ccc3c(c2)-c2cccc4cccc-3c24)c1 ; N#Cc1cc(-c2cccc(-c3ccc(-c4nc(-c5ccccc5)nc(-c5ccc(-c6ccc7c8c(cccc68)-c6ccccc6-7)cc5)n4)cc3)c2)ccc1-c1cccc(-c2cc3ccccc3c3ccccc23)c1 ; N#Cc1cc(-c2ccccc2-c2cccc(-c3nc(-c4ccccc4)nc(-c4cccc(-c5ccc6ccccc6c5)c4)n3)c2)ccc1-c1cccc(-c2ccc3c(c2)-c2cccc4cccc-3c24)c1]

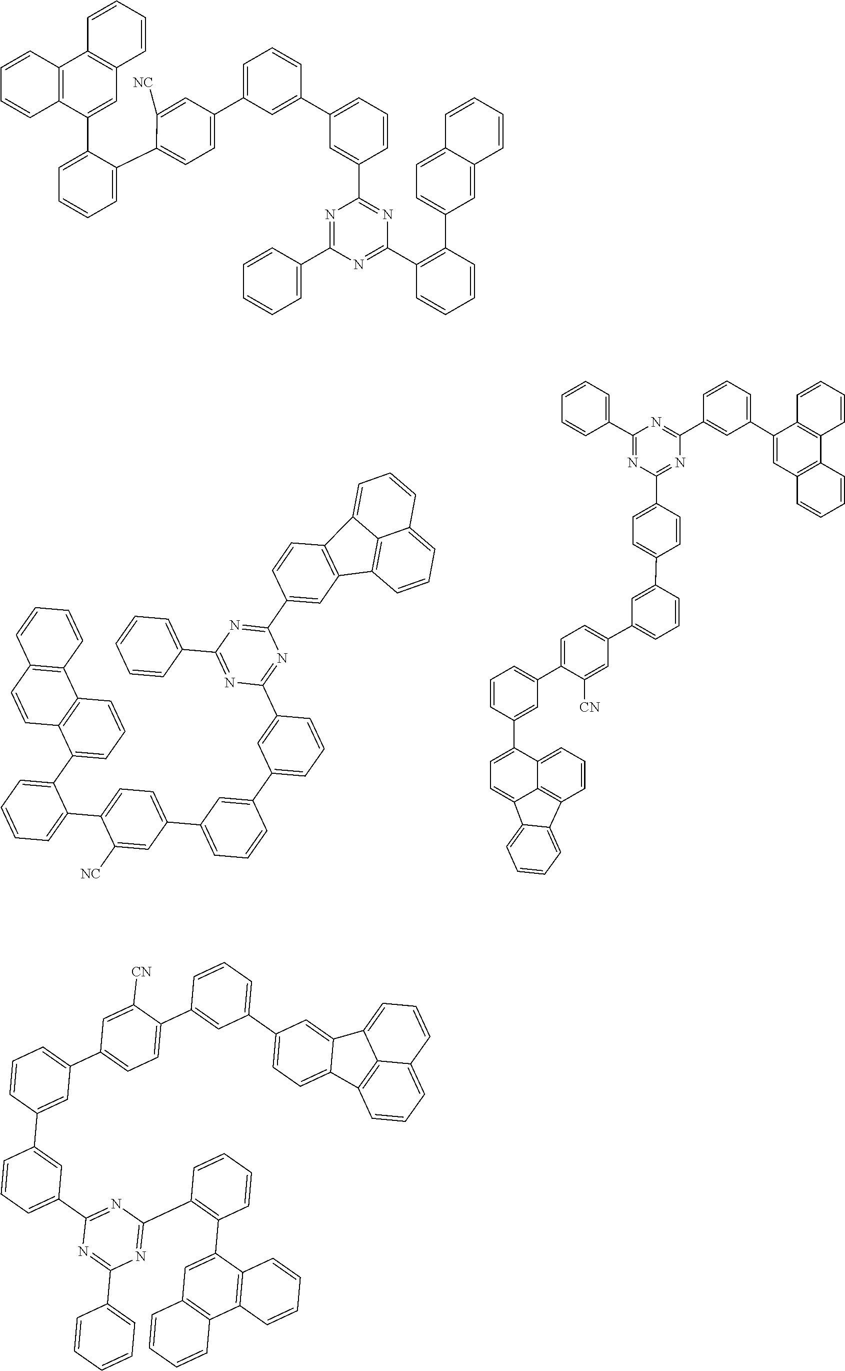 [SMILES: N#Cc1cc(-c2cccc(-c3cccc(-c4nc(-c5ccccc5)nc(-c5ccccc5-c5ccc6ccccc6c5)n4)c3)c2)ccc1-c1ccccc1-c1cc2ccccc2c2ccccc12 ; N#Cc1cc(-c2cccc(-c3cccc(-c4nc(-c5ccccc5)nc(-c5ccc6c(c5)-c5cccc7cccc-6c57)n4)c3)c2)ccc1-c1ccccc1-c1cccc2c1ccc1ccccc12 ; N#Cc1cc(-c2cccc(-c3ccc(-c4nc(-c5ccccc5)nc(-c5cccc(-c6cc7ccccc7c7ccccc67)c5)n4)cc3)c2)ccc1-c1cccc(-c2ccc3c4c(cccc24)-c2ccccc2-3)c1 ; N#Cc1cc(-c2cccc(-c3cccc(-c4nc(-c5ccccc5)nc(-c5ccccc5-c5cc6ccccc6c6ccccc56)n4)c3)c2)ccc1-c1cccc(-c2ccc3c(c2)-c2cccc4cccc-3c24)c1]

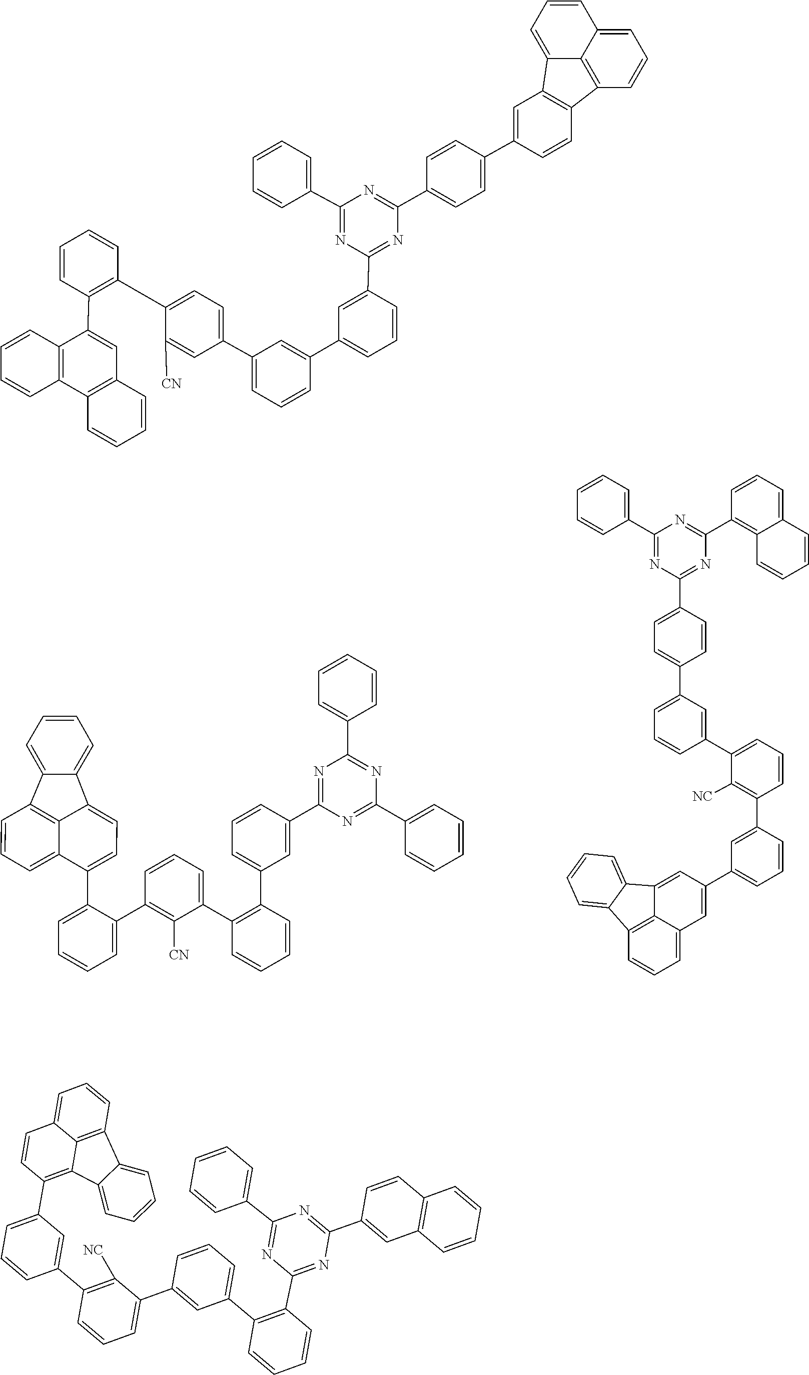 [SMILES: N#Cc1cc(-c2cccc(-c3cccc(-c4nc(-c5ccccc5)nc(-c5ccc(-c6ccc7c(c6)-c6cccc8cccc-7c68)cc5)n4)c3)c2)ccc1-c1ccccc1-c1cc2ccccc2c2ccccc12 ; N#Cc1c(-c2ccccc2-c2cccc(-c3nc(-c4ccccc4)nc(-c4ccccc4)n3)c2)cccc1-c1ccccc1-c1ccc2c3c(cccc13)-c1ccccc1-2 ; N#Cc1c(-c2cccc(-c3ccc(-c4nc(-c5ccccc5)nc(-c5cccc6ccccc56)n4)cc3)c2)cccc1-c1cccc(-c2cc3c4c(cccc4c2)-c2ccccc2-3)c1 ; N#Cc1c(-c2cccc(-c3ccccc3-c3nc(-c4ccccc4)nc(-c4ccc5ccccc5c4)n3)c2)cccc1-c1cccc(-c2ccc3cccc4c3c2-c2ccccc2-4)c1]

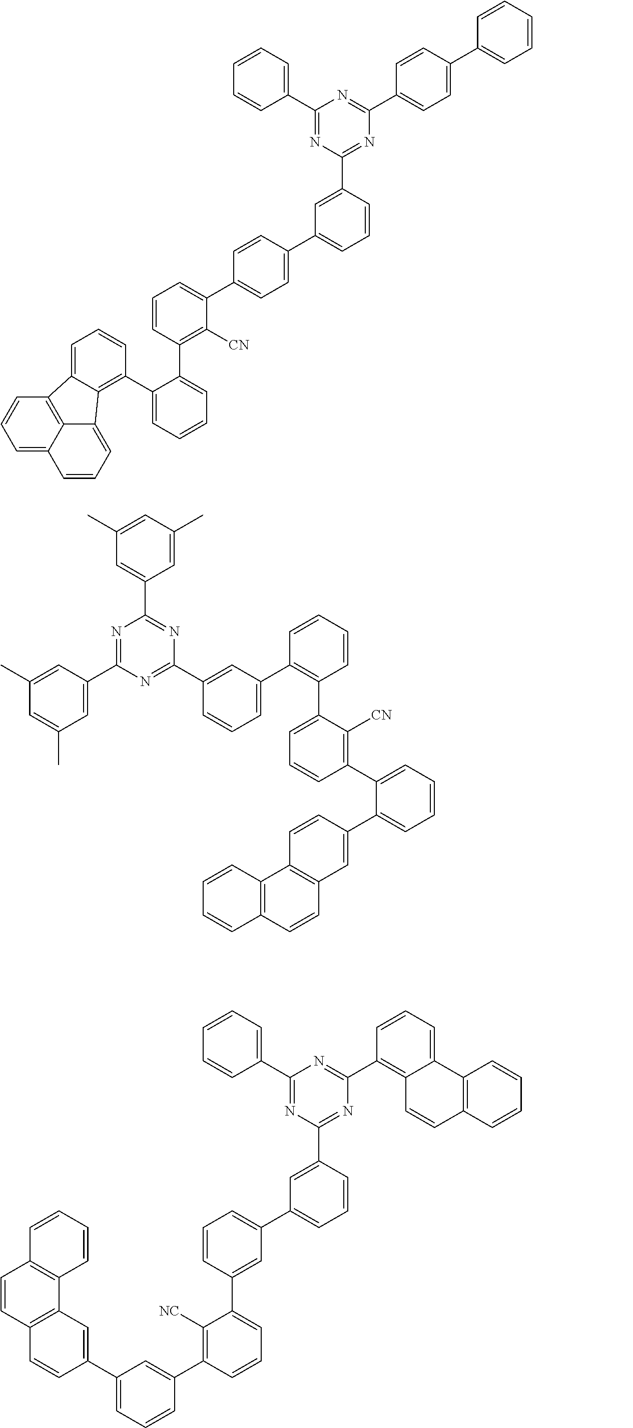

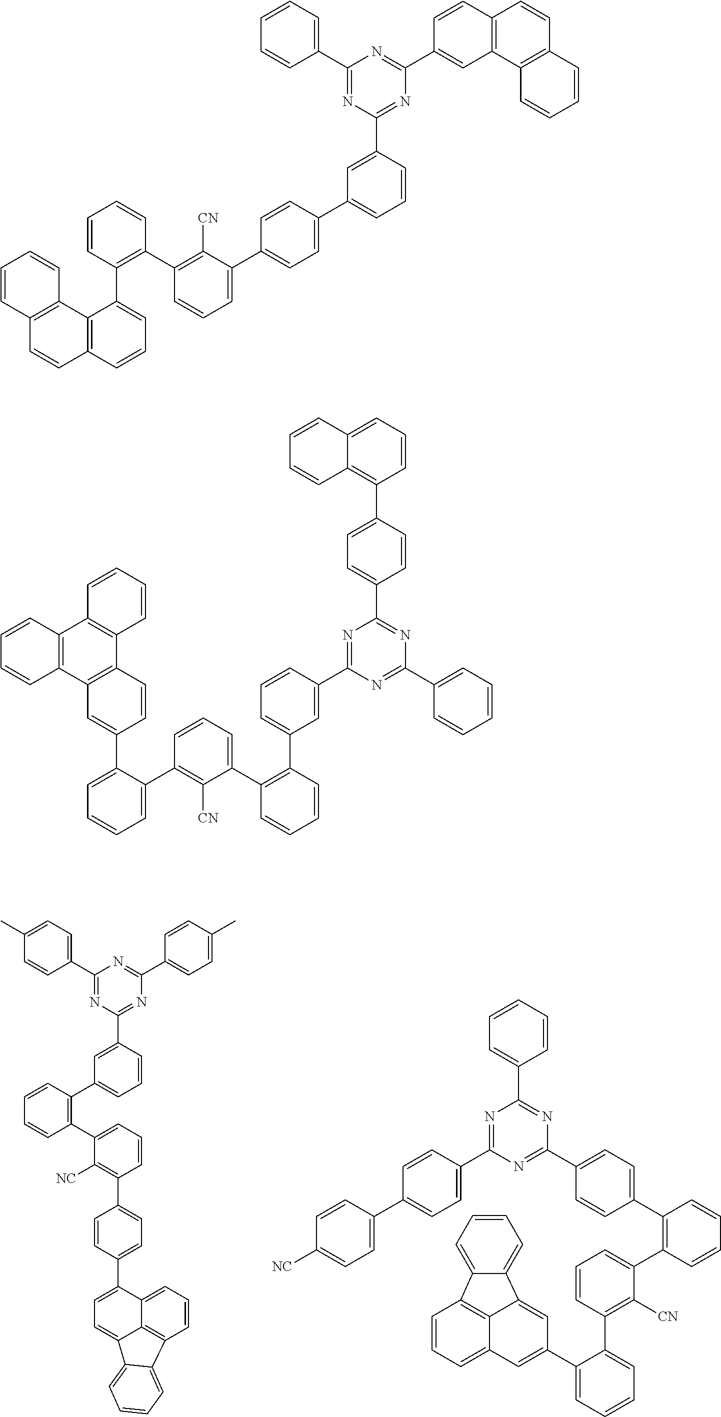

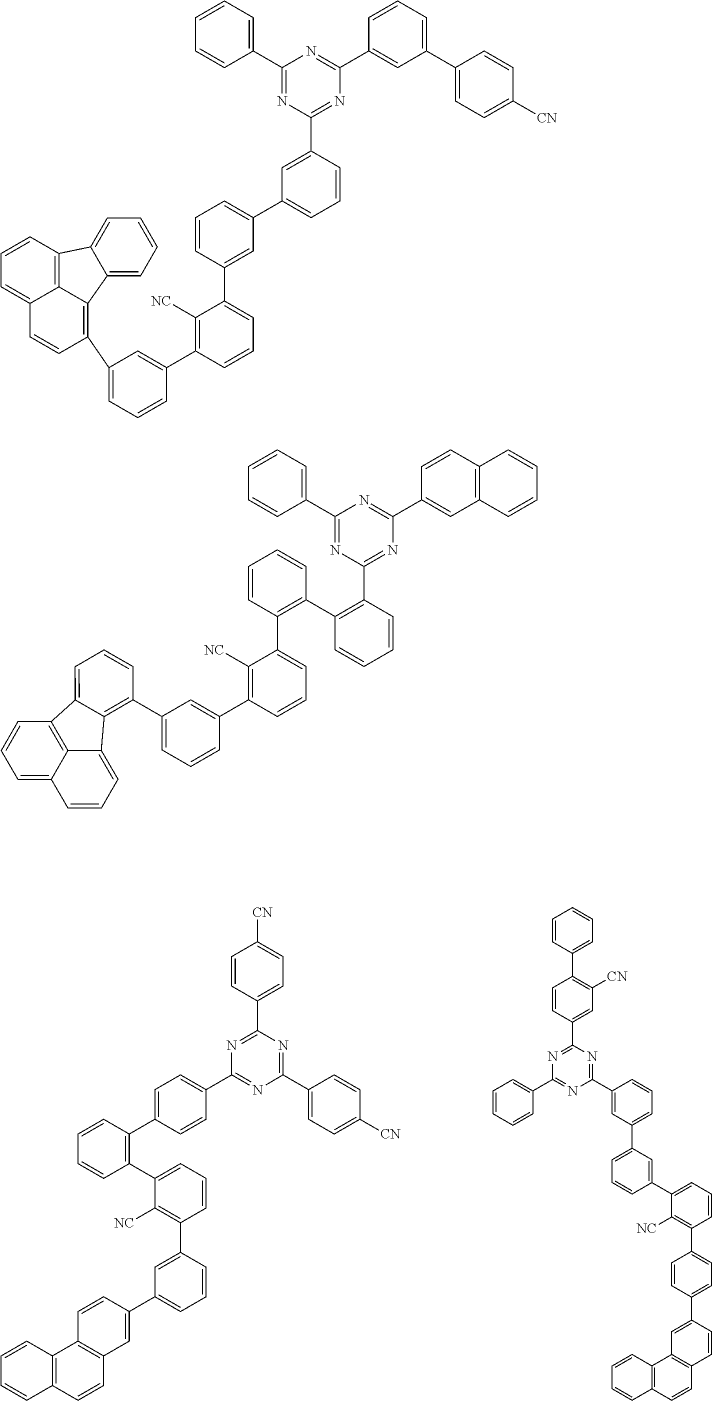

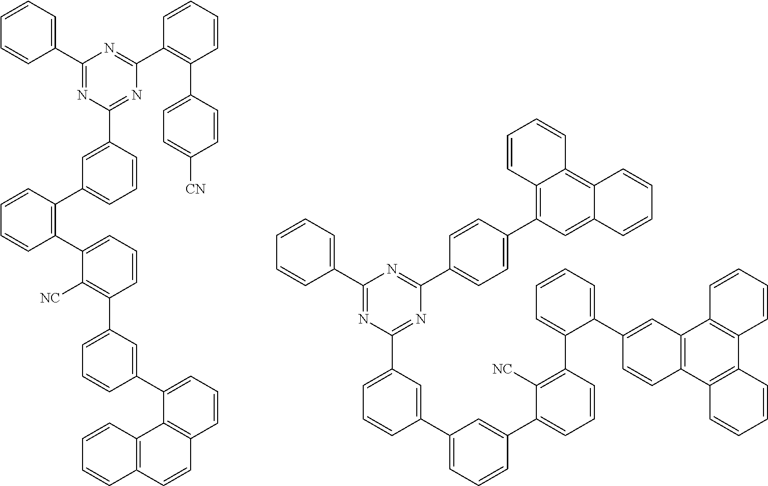

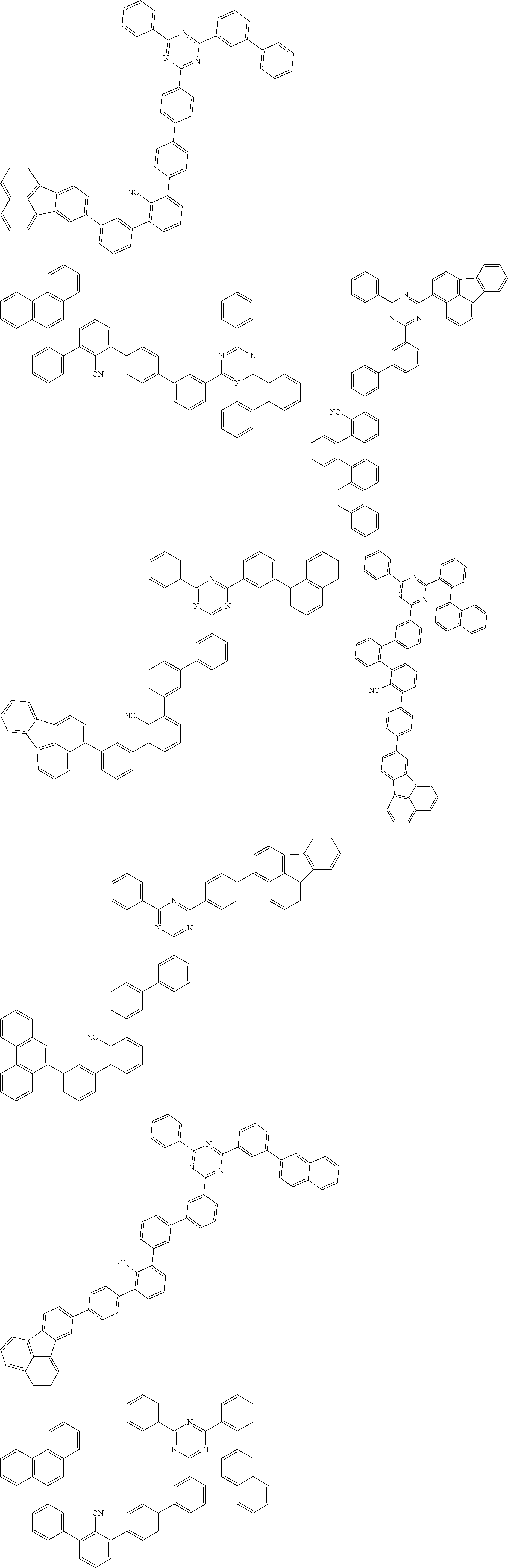

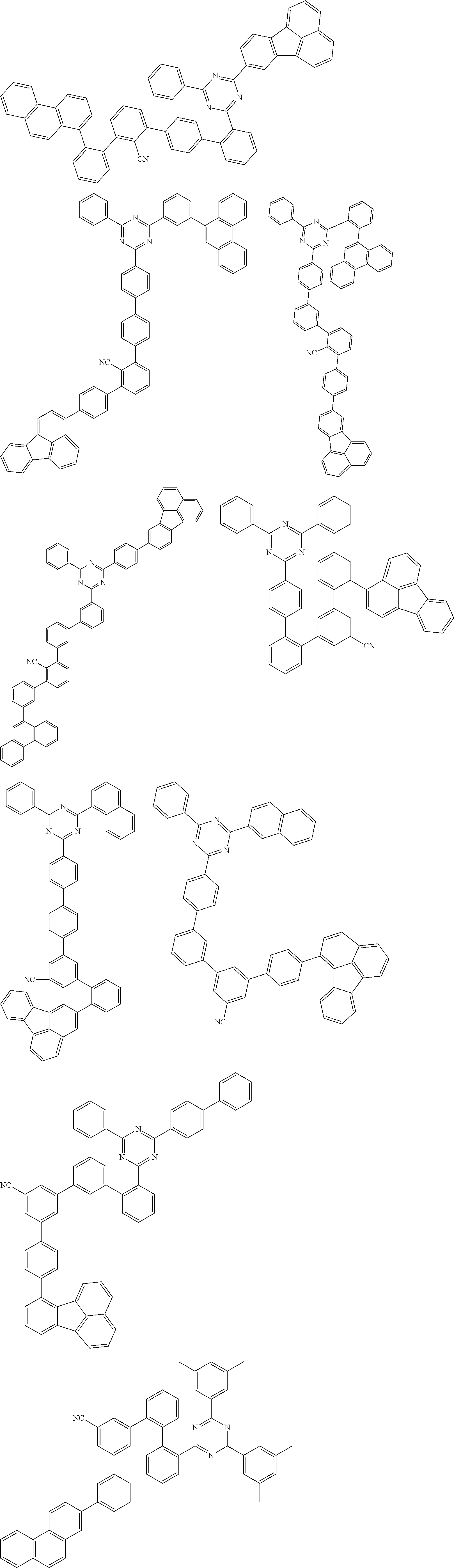

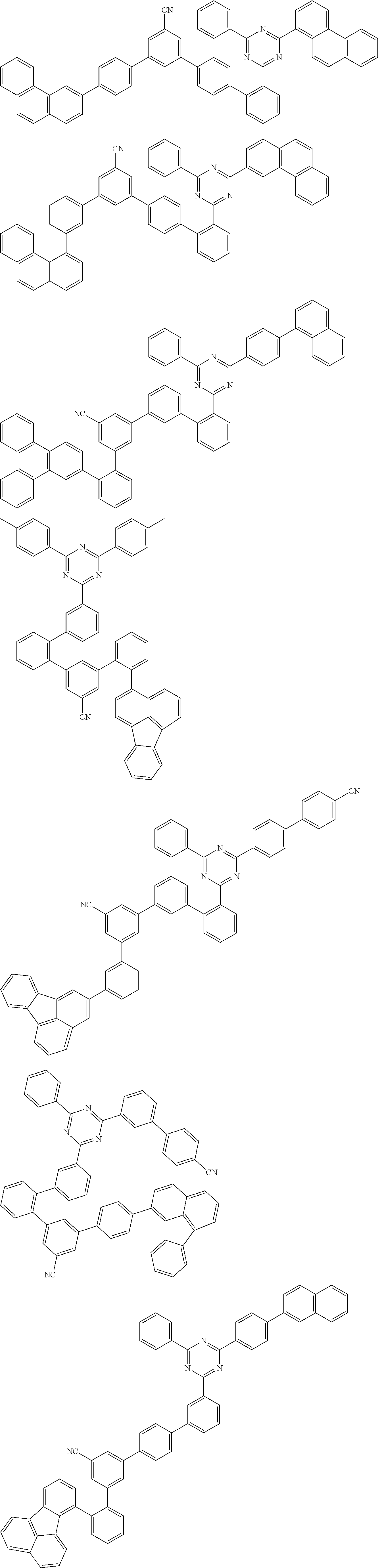

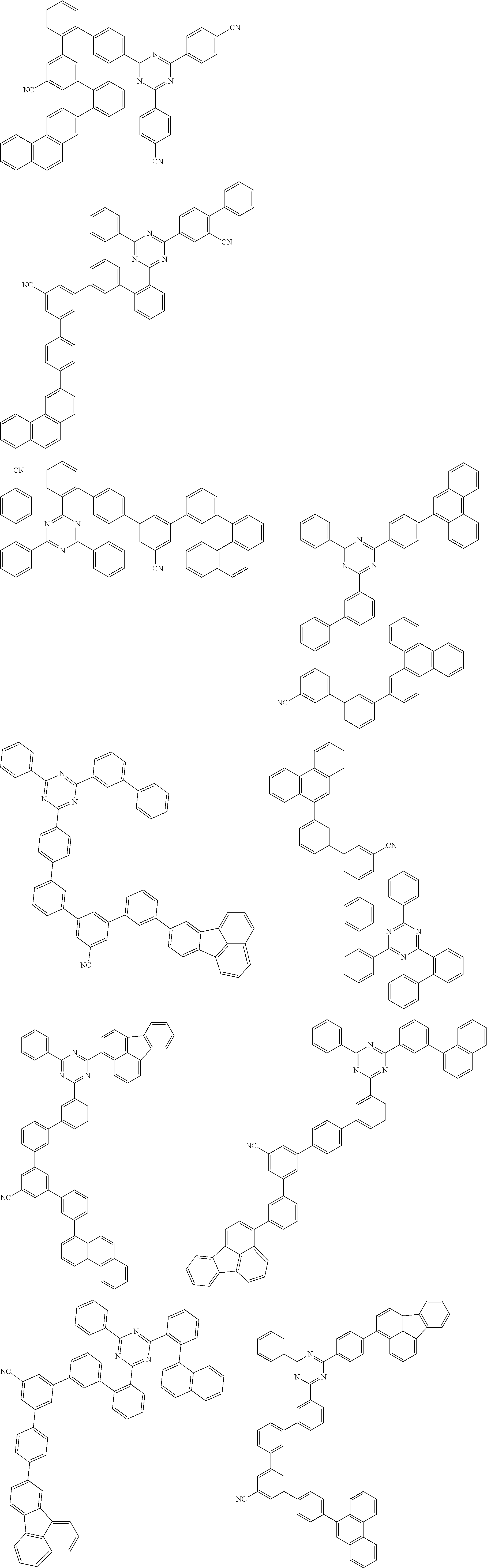

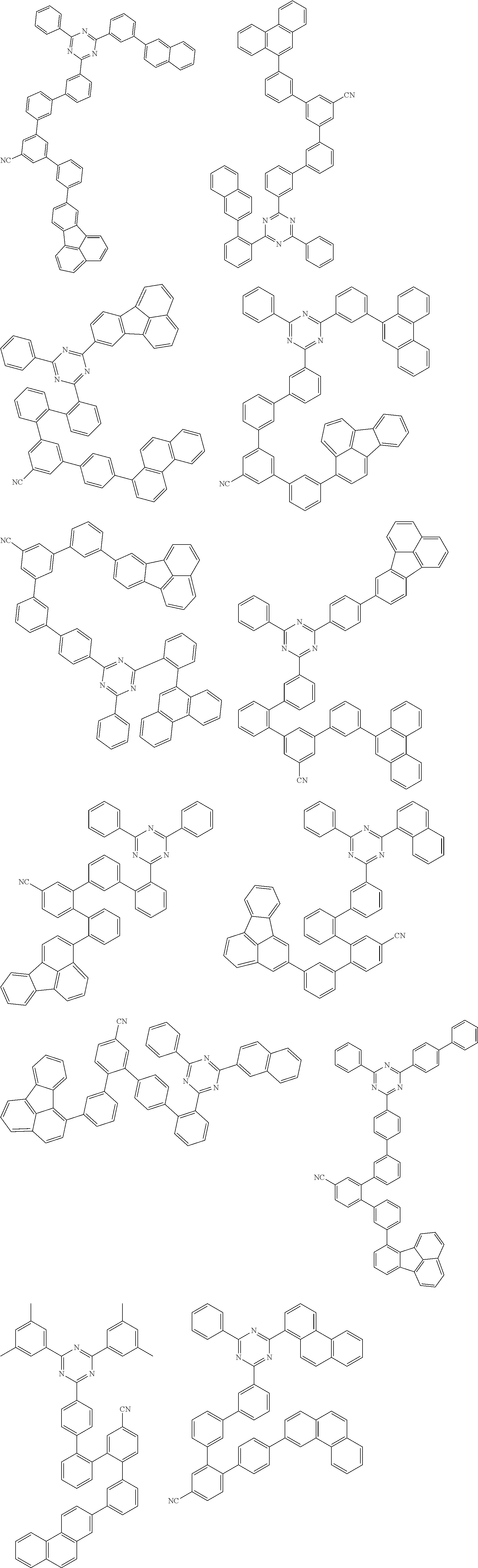

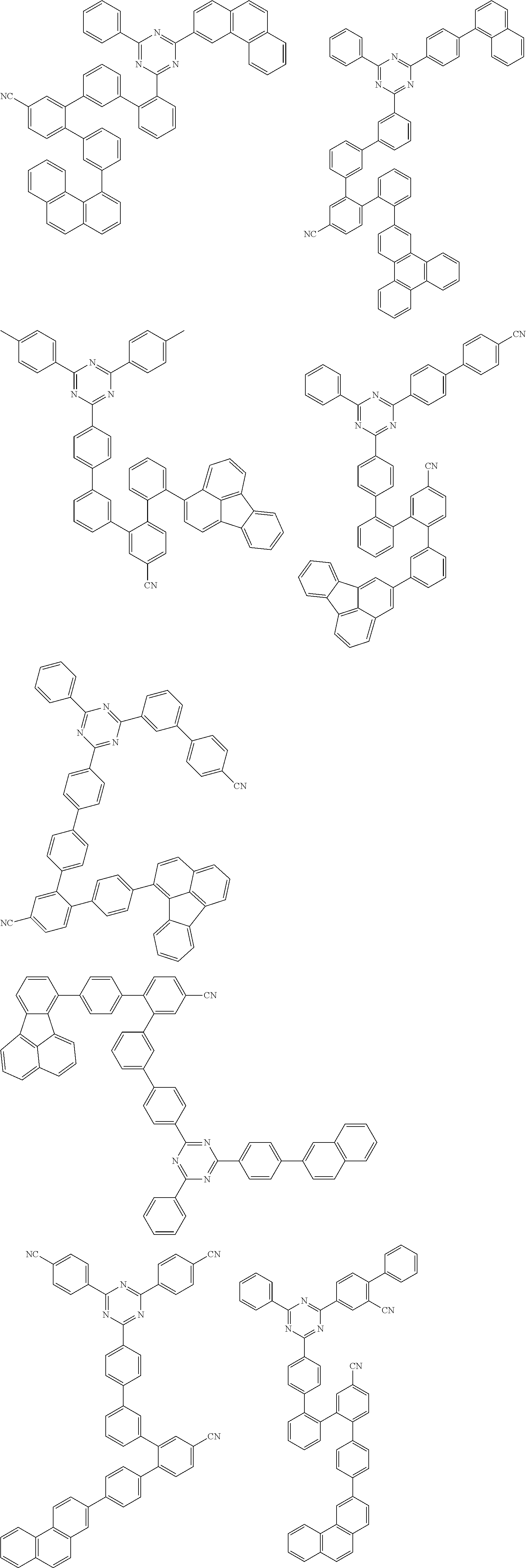

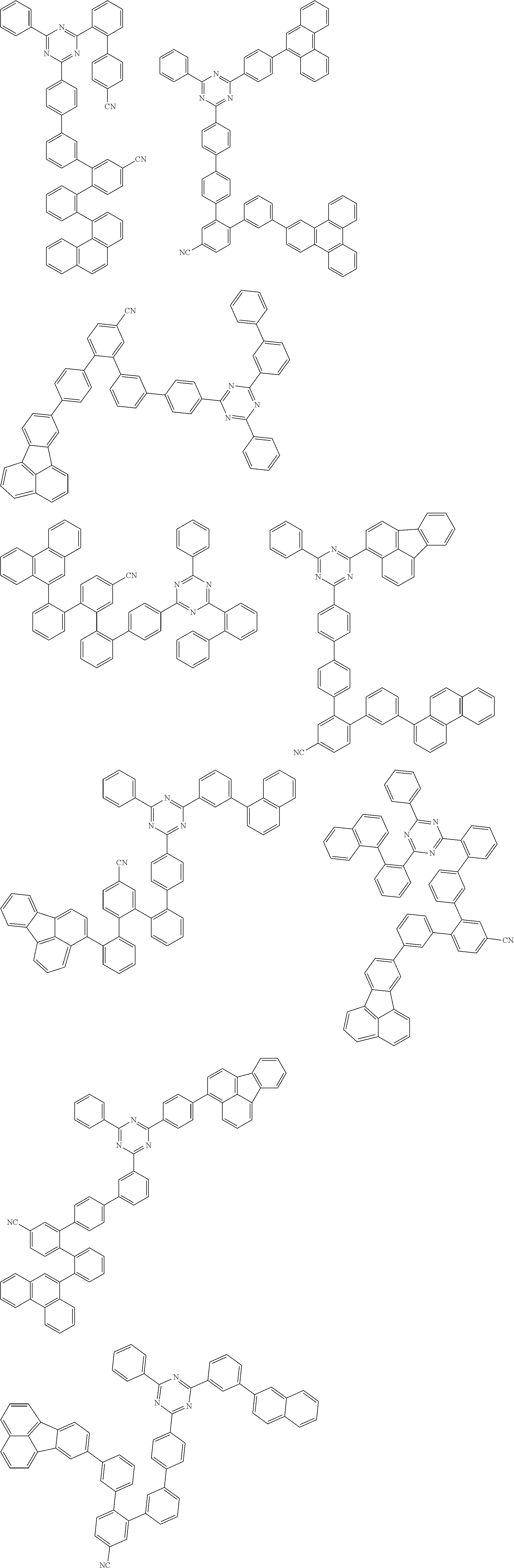

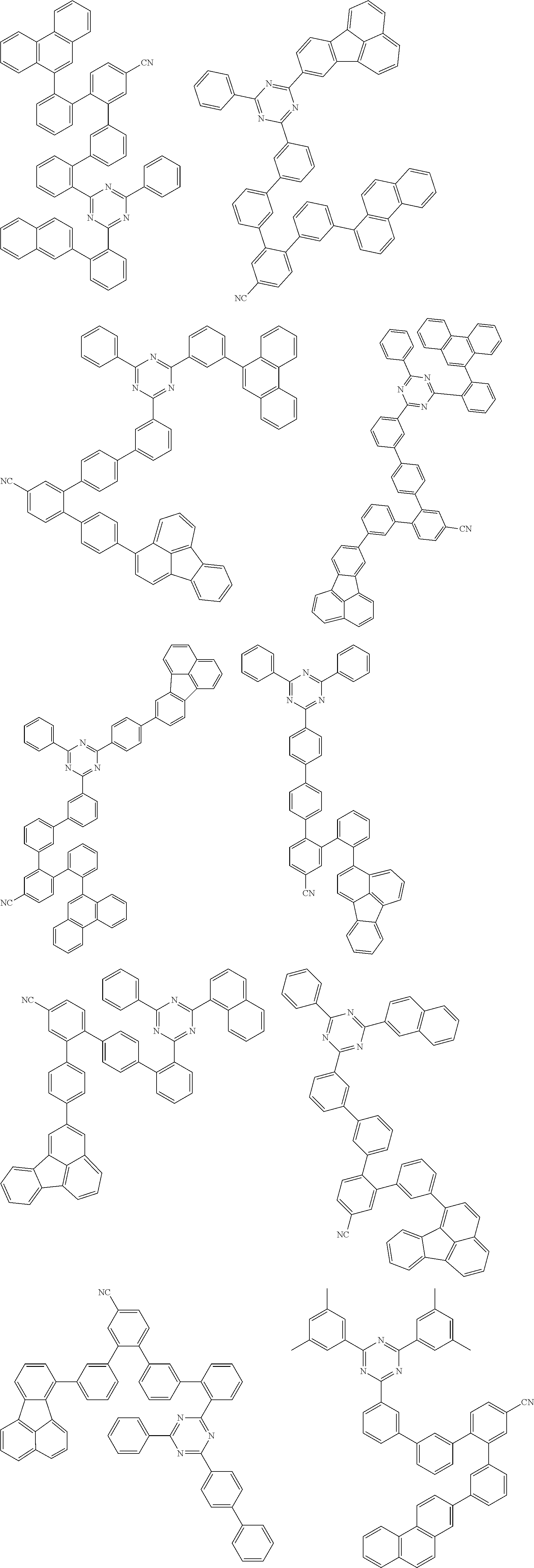

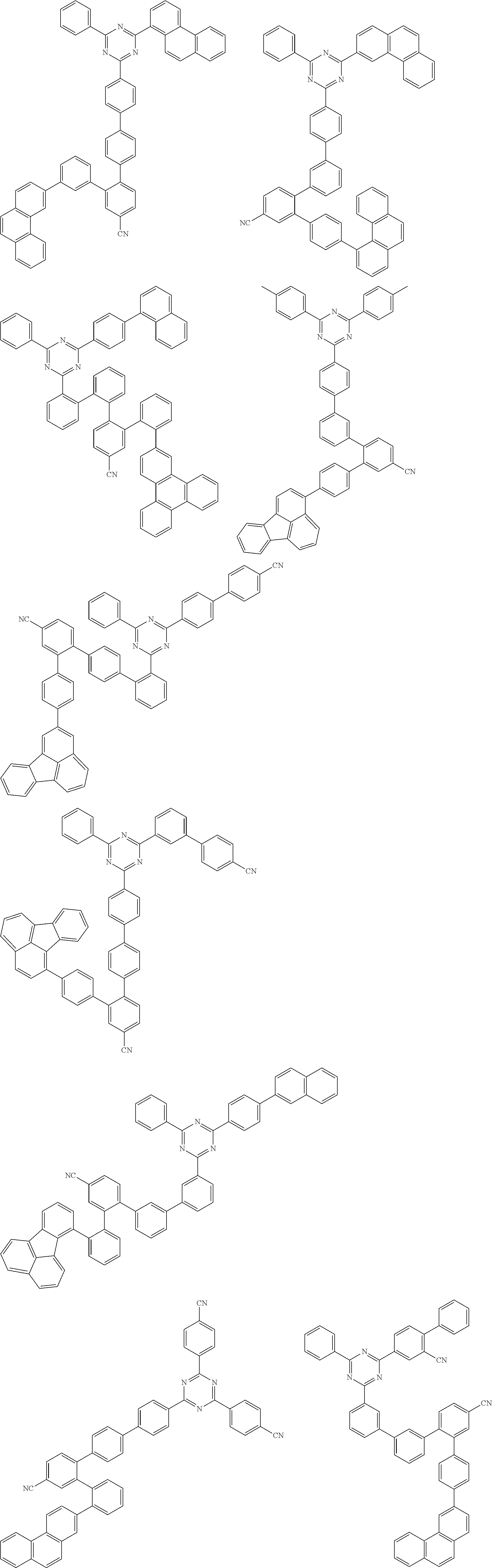

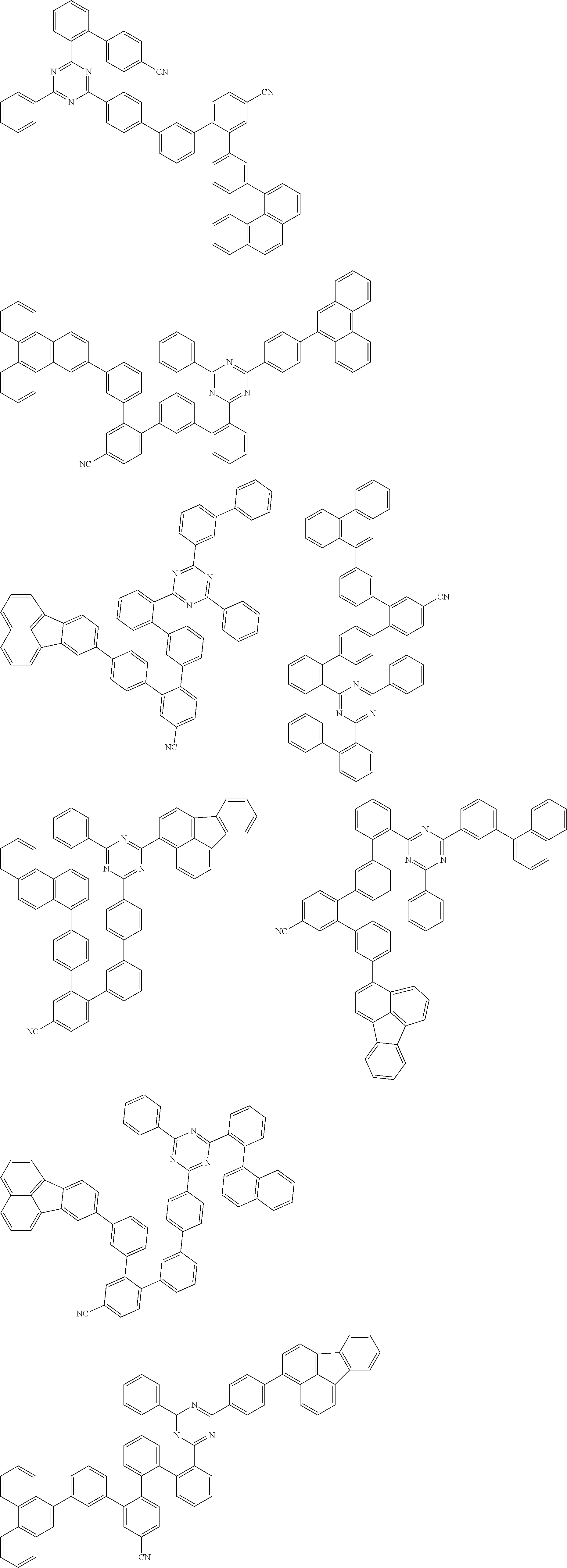

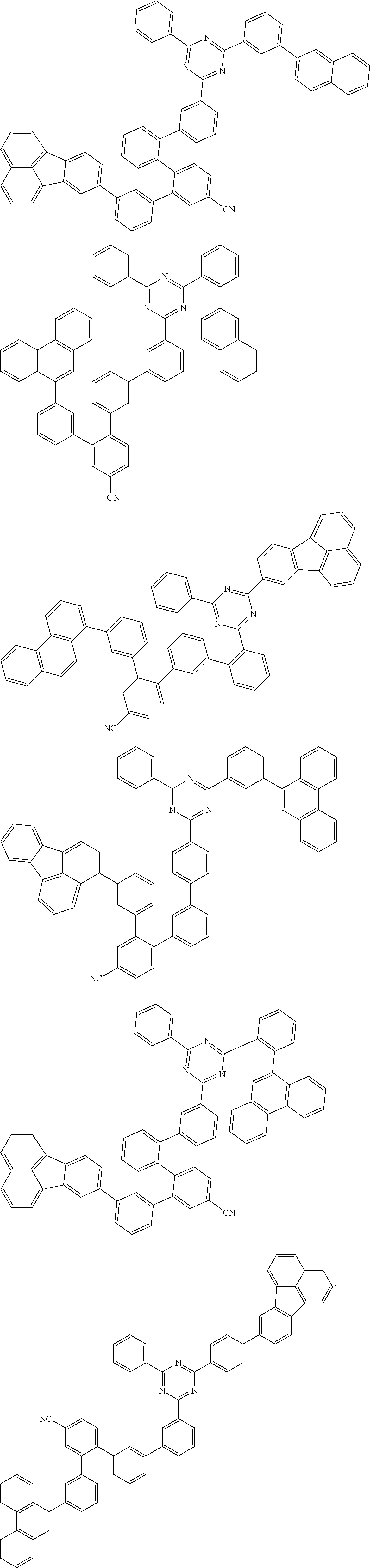

14. An organic light emitting device, comprising:
a first electrode;
a second electrode that is opposite to the first electrode; and
one or more organic material layers that are between the first electrode and the second electrode, wherein at least one organic material layer of the one or more organic material layers comprises the compound according to claim 1.

15. The organic light emitting device of claim 14, wherein the organic material layer comprising the compound is a hole blocking layer, an electron transport layer, or an electron injection and transport layer.
